(19) United States
(12) Patent Application Publication
(10) Pub. No.: US 2026/0262373 A1
(43) Pub. Date: Sep. 3, 2026

(54) HELICALLY CHIRAL PLATINUM(II) AND PALLADIUM(II) COMPLEX CIRCULARLY POLARIZED LUMINESCENT MATERIALS AND THEIR APPLICATIONS

(71) Applicant: ZHEJIANG UNIVERSITY OF TECHNOLOGY, Hangzhou, Zhejiang (CN)

(72) Inventors: Guijie LI, Hangzhou (CN); Kewei XU, Hangzhou (CN); Yuanbin SHE, Hangzhou (CN); Shun LIU, Hangzhou (CN)

(21) Appl. No.: 18/994,000

(22) PCT Filed: Jul. 24, 2023
(86) PCT No.: PCT/CN2023/108907

§ 371 Date: Jan. 13, 2025

(30) Foreign Application Priority Data
Jul. 25, 2022 (CN) 202210876863.X

(51) Int. Cl. H10K 50/12; C09K 11/06; H10K 50/80; H10K 85/30; H10K 101/30

(52) U.S. Cl. CPC H10K 50/12; C09K 11/06; H10K 50/868; H10K 85/346; C09K 2211/185; H10K 2101/30

(57) ABSTRACT

Disclosed are a helically chiral platinum(II) or palladium(II) complex-based circularly polarized luminescent material, and a use thereof. The formation of the helically chiral metal complex molecule can autonomously induce an entire tetradentate ligand to coordinate with a metal ion in a manner having minimal steric hindrance by means of a central chiral fragment in the ligand, to form an optically pure helically chiral metal complex circularly polarized luminescent material, the induction reaction being stereospecific; the helically chiral metal complex does not require chiral resolution; the helically chiral metal complex material has high chemical stability and thermal stability. The present helically chiral metal complex has important uses in circularly polarized light-emitting components and related fields.

### CROSS REFERENCE

This application is a national phase of International Application No. PCT/CN2023/108907, filed on Jul. 24, 2023, which claims priority under 35 U.S.C. § 119(a) to and benefit of Chinese Patent Application No. 202210876863.X, filed on Jul. 25, 2022. Both the above-referenced applications are incorporated into the present application by reference herein in their entirety.

### TECHNICAL FIELD

The disclosure relates to the technical field of circularly polarized luminescence materials and their applications, in particular to a circularly polarized luminescent material of helically chiral tetradentate cyclometalated platinum(II) and palladium(II) complexes based on benzocarbene and its derivative structural units and their applications.

### BACKGROUND ART

Circularly polarized luminescence (CPL) is a form of luminescence that is completely different from the sunlight which is linear luminescence (see FIG. 1). Circularly polarized luminescence is a phenomenon in which chiral luminescent materials emit left-handed or right-handed circularly polarized light after being excited (see FIG. 2). Therefore, the design and development of chiral luminescent materials are crucial in this field. With the in-depth research of researchers, circularly polarized luminescent materials have important applications in fields such as 3D display, data storage, quantum computing, optical anti-counterfeiting, biological imaging, and asymmetric synthesis.

Cyclometalated phosphorescent platinum(II) and palladium(II) complex materials, due to their heavy atom effect, can fully utilize electroexcitation to generate all singlet and triplet excitons, resulting in a maximum theoretical quantum efficiency of up to 100%. Therefore, such complexes are an ideal class of luminescent materials. The rigidity of platinum(II) and palladium(II) complexes with bidentate ligands is relatively low. Due to the twisting and vibration of the two bidentate ligands, the energy of excited material molecules is consumed in a nonradiative manner, resulting in a decrease in their luminescence quantum efficiency (Inorg. Chem. 2010, 49, 11276), however, the presence of the second monodentate ligand (such as Cl⁻, phenoxy anion, alkyne anion, carbene, etc.) greatly reduces the chemical and thermal stabilities of the complexes, making them difficult to sublimate and purify for the preparation of OLED device. Therefore, the luminescent materials based on bidentate and tridentate ligand ring metal complexes are not conducive to their application in stable and efficient OLED devices. The central metal ions of the divalent cyclometalated platinum(II) and palladium(II) complexes are both dsp² hybridized, making them easy to coordinate with tetradentate ligands to form stable and rigid planar quadrilateral configuration molecules; High molecular rigidity can suppress nonradiative relaxation caused by molecular vibration and rotation, reduce energy loss of excited state material molecules, and thus improve the quantum efficiency of material molecule luminescence. Due to the steric hindrance of the two aryl groups at the end of the tetradentate ligand of cyclometalated platinum(II) and palladium(II) complexes, the material molecules exhibit a twisted quadrilateral configuration (Chem. Mater. 2020, 32, 537), theoretically possessing helical chirality. However, the molecules are easily racemized by the up and down vibrations of the two aryl groups at the end of the ligand in solution or during heating and sublimation, making it impossible to separate their enantiomers and obtain optically pure cyclometalated platinum(II) and palladium(II) complex material molecules, which do not exhibit circularly polarized luminescence properties. Therefore, the design and development of optically pure cyclometalated platinum(II) and palladium(II) complexes with high chemical and thermal stabilities, as well as circularly polarized luminescent properties, is of great significance and practical value for their application in circularly polarized luminescent OLED devices (CP-OLED), and is also an urgent problem to be solved in the CP-OLED field.

### SUMMARY

The purpose of the present disclosure is to provide a helically chiral tetradentate cyclometalated platinum(II) and palladium(II) complexes based on benzocarbene and its derivative structural units and their applications as circularly polarized photoluminescence materials, in response to the shortcomings of existing technology, and its application in devices.

The entire tetradentate ligands can autonomously coordinate with metal ions in a low steric hindrance manner induced by the central chiral fragment in the tetradentate ligands, which autonomously form optically pure helically chiral cyclometalated platinum(II) or palladium(II) complexes circularly polarized light emitting materials without chiral separation. The materials have high chemical and thermal stabilities and important applications in circularly polarized light emitting devices.

The purpose of the present disclosure is achieved through the following technical solutions; a circularly polarized luminescent material with a central chiral induced helically chiral tetradentate cyclometalated platinum(II) and palladium(II) complexes, whose chemical formulas are shown in general formulas (I) and (I′), where (I) and (I′) are enantiomers of each other:

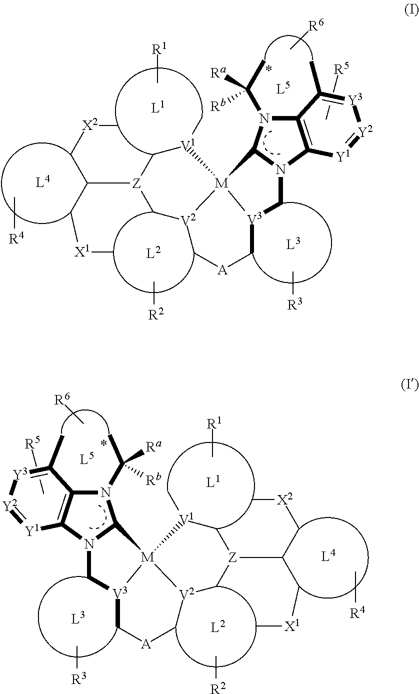

where M is Pt or Pd; V¹, V², and V³ are each independent N or C; Y¹, Y², and Y³ are each independent as N or C;
L¹, L², L³, and L⁴ are each independent as five membered or six membered carbon ring, heterocycle, aromatic ring, or heteroaromatic ring; L⁵ is a six to ten membered carbon ring or heterocycle containing central chirality, where “*” represents a carbon atom with central chirality, that is, R^{a} and R^{b} are different substituents in the same molecule;
A is O, S, CR^{x}R^{y}, C═O, SiR^{x}R^{y}, GeR^{x}R^{y}, NR^{z}, PR^{z}, R^{z}P═O, AsR^{z}, R^{z}As═O, S═O, SO₂, Se, Se═O, SeO₂, BH, BR^{z}, R^{z}Bi═O or BiR^{z};
X¹ and X² can exist or not exist, and if they exist, X¹ and X² are each independently single bond, O, S, CR^{x}R^{y}, C═O, SiR^{x}R^{y}, GeR^{x}R^{y}, NR^{z}, PR^{z}, R^{z}P═O, AsR^{z}, R^{z}As═O, S═O, SO₂, Se, Se═O, SeO₂, BH, BR^{z}, R^{z}Bi═O or BiR^{z};
Z is N, CR^{x}, SiR^{x}, GeR^{x}, B, P, P═O, As, As═O, Bi═O or Bi;
R¹, R², R³, R⁴, R⁵ and R⁶ each independently representing single, double, three, four, five, or six substituted or unsubstituted, and meanwhile, R¹, R², R³, R⁴, R⁵, R⁶, R^{a}, R^{b}, R^{x}, R^{y} and R^{z} are each independently hydrogen, deuterium, halogen, alkyl, cycloalkyl, aryl, heterocycloalkyl, heteroaryl, haloalkyl, haloaryl, haloheteroaryl, alkoxy, aryloxy, alksiliyl, arylsiliyl, heteroarylsiliyl, alkyl (hetero) arylsiliyl, alkenyl, cycloalkenyl, alkynyl, hydroxyl, mercapto, nitro, cyano, amino, mono- or dialkylamino, mono- or diarylamino, ester, isonitrile, alkoxycarbonyl, amido, alkoxycarbonylamino, aryloxycarbonylamino, sulfonylamino, sulfamoyl, carbamoyl, alkylthio, sulfinyl, ureido, phosphonamido, imino, sulfo, carboxyl, hydrazino, or combinations thereof, two or more adjacent R¹, R², R³, R⁴, R⁵ and R⁶ can selectively connect to form a fused ring; any two groups in R^{a}, R^{b}, and R⁶ can be connected to form a cyclic system.

The above-mentioned helically chiral circularly polarized luminescent material platinum(II) and palladium(II) complexes in general formulas (I) and (I′), and L³ in the general structure can be the following structure, but not limited to this:

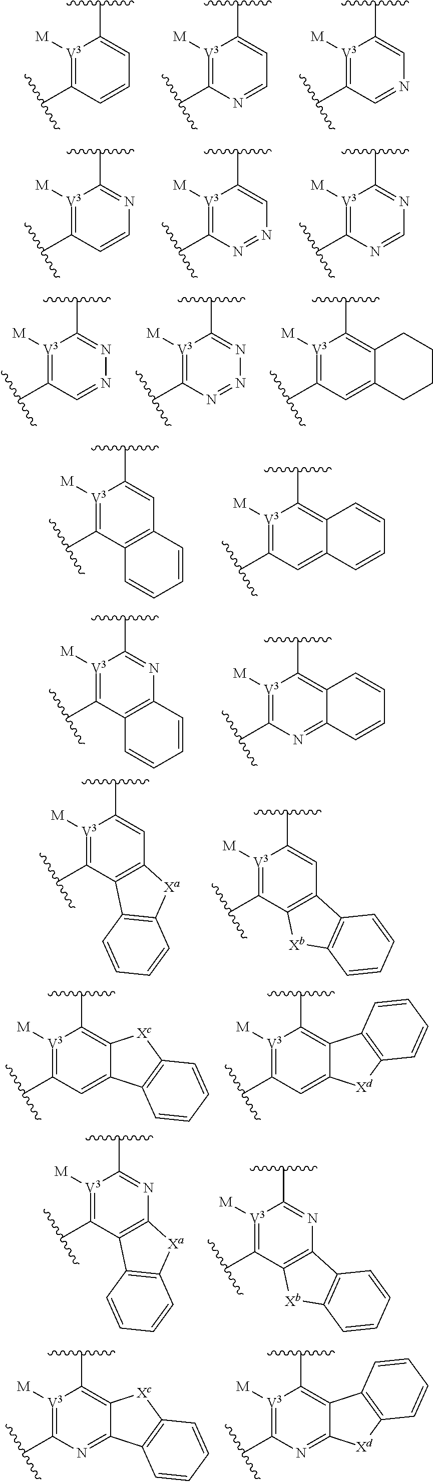

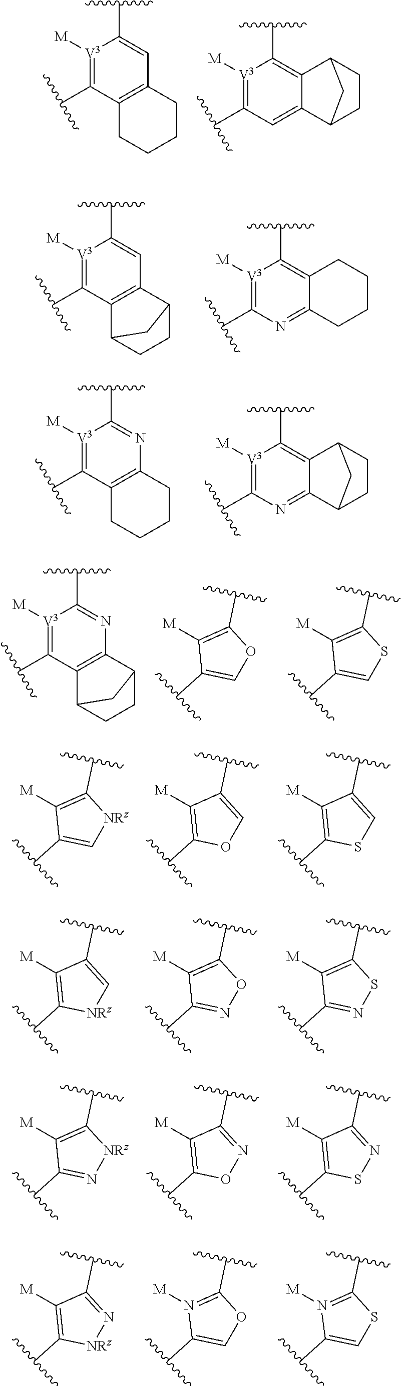

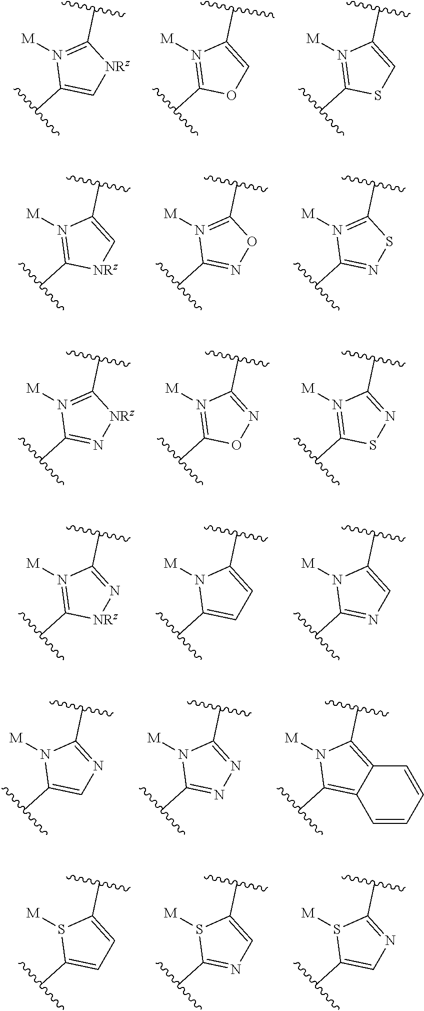

wherein, X^{a}, X^{b}, X^{c}, and X^{d} are each independently single bond, O, S, CR^{x}R^{y}, C═O, SiR^{x}R^{y}, GeR^{x}R^{y}, NR^{z}, PR^{z}, R^{z}P═O, AsR^{z}, R^{z}As═O, S═O, SO₂, Se, Se═O, SeO₂, BH, BR^{z}, R^{z}Bi═O or BiR^{z}.

The above-mentioned helically chiral circularly polarized luminescent material platinum(II) and palladium(II) complexes based on the structural units of benzocarbene and its derivatives in general formulas (I) and (I′), preferably, one of the following general formulas (I-A), (I-B), (I-C), (I-D), (I-E), (I-F), (I-G), (I-H), (I-I) and their enantiomers (I′-A), (I′-B), (I′-Q, (I′-D), (I′-E), (I′-F), (I′-G), (I′-H), and (I′-I) is preferred, but not limited to this:

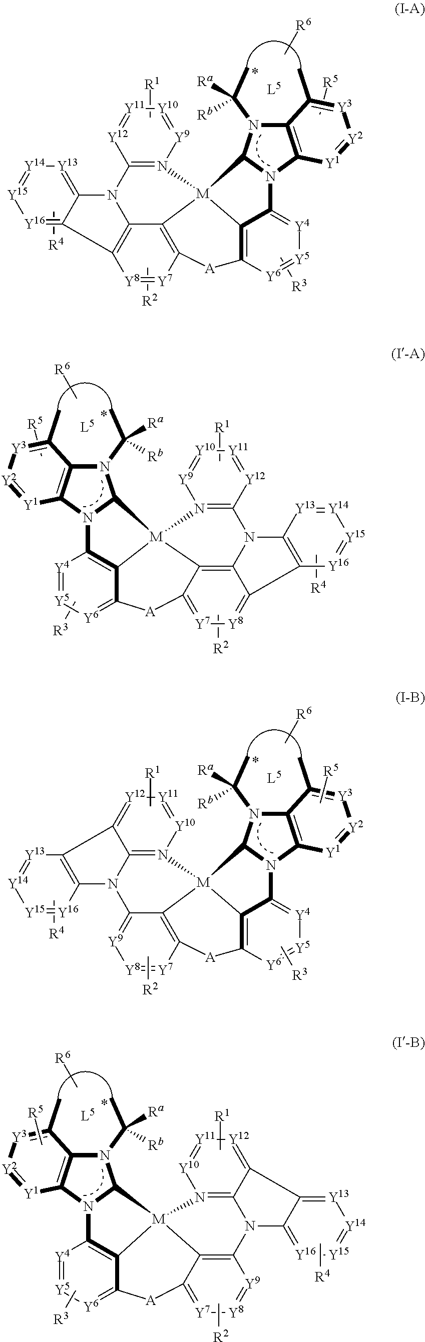

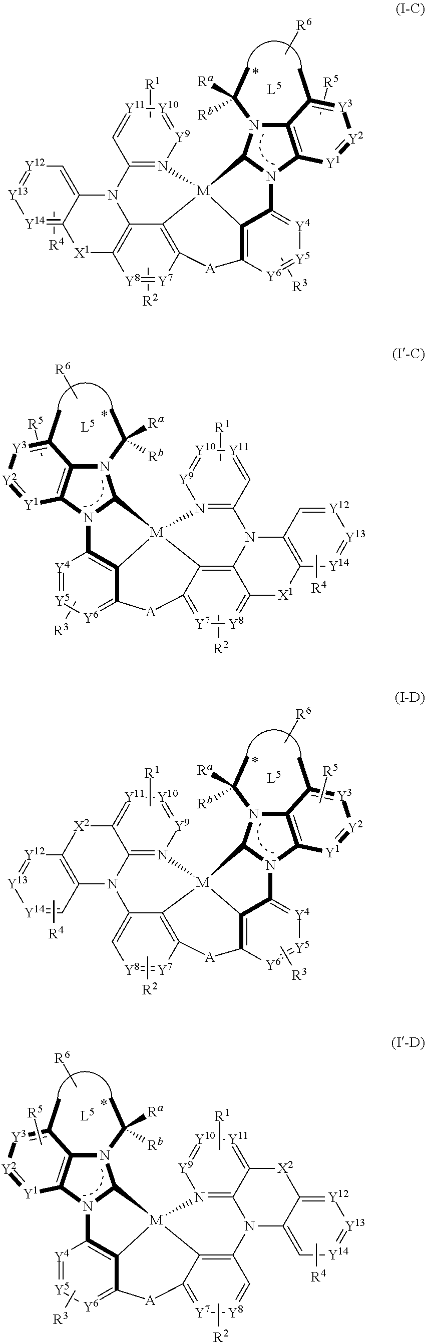

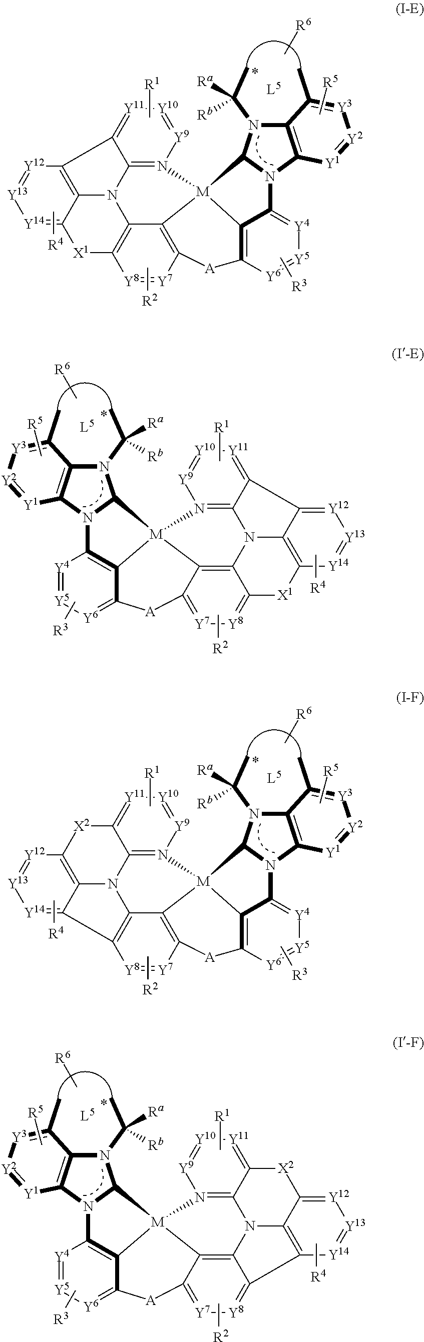

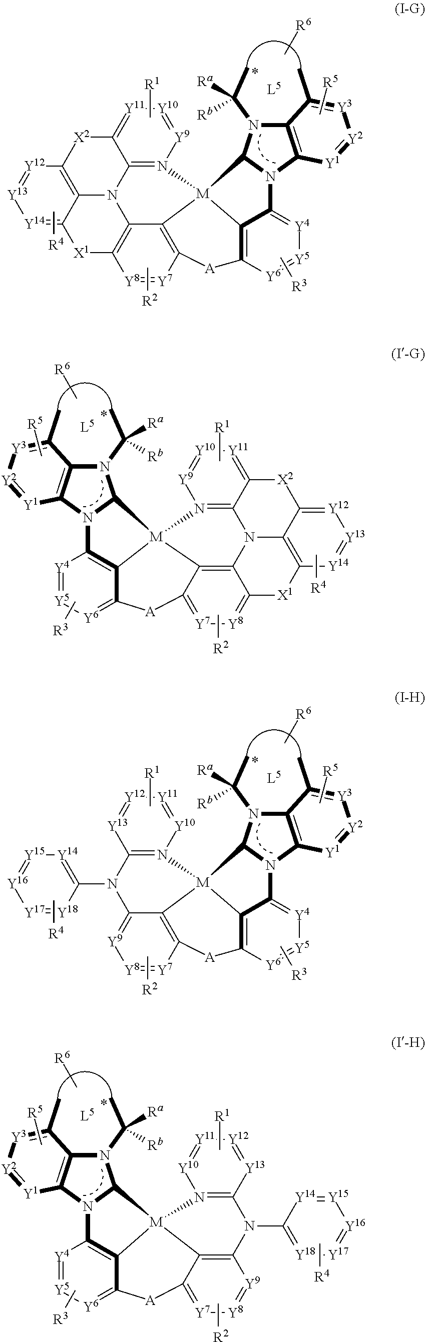

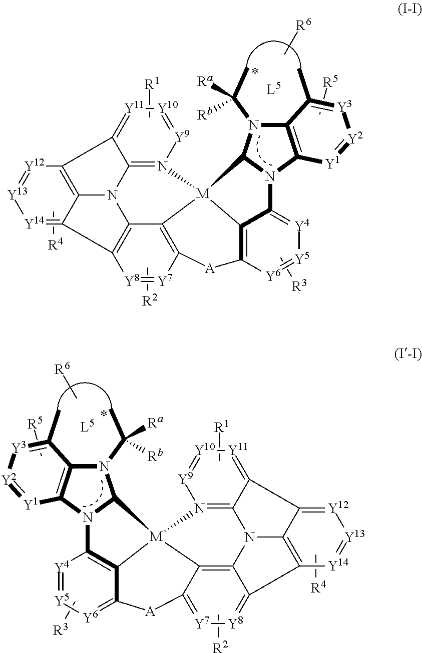

Y⁴, Y⁵, Y⁶, Y⁷, Y⁸, Y⁹, Y¹⁰, Y¹¹, Y¹², Y¹³, Y¹⁴, Y¹⁵ and Y¹⁶ are each independently N or C.

The L⁵ in the general formula structure of the helically chiral tetradentate cyclometalated to platinum(II) and palladium(II) complexes based on the structural units of benzocarbene and its derivatives is selected from the following structures and their enantiomers, but not limited to them:

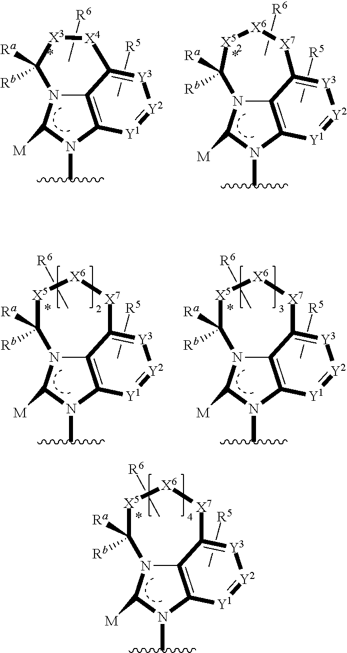

wherein X³, X⁴, X⁵, X⁶ and X⁷ are each independently O, S, CR^{x}R^{y},

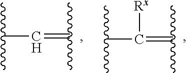

C═O, SiR^{x}R^{y}, GeR^{x}R^{y}, NR^{z}, PR^{z}, R^{z}P═O, AsR^{z}, R^{z}As═O, S═O, SO₂, Se, Se═O, SeO₂, BH, BR^{z}, R^{z}Bi═O or BiR^{z};
The structure of L⁵ is preferred from the following structures and their enantiomers, but not limited to them:

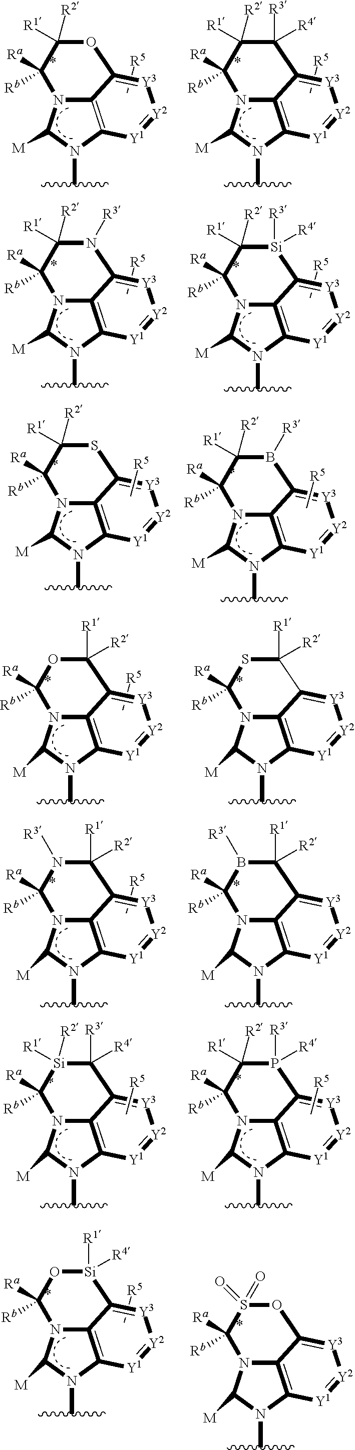

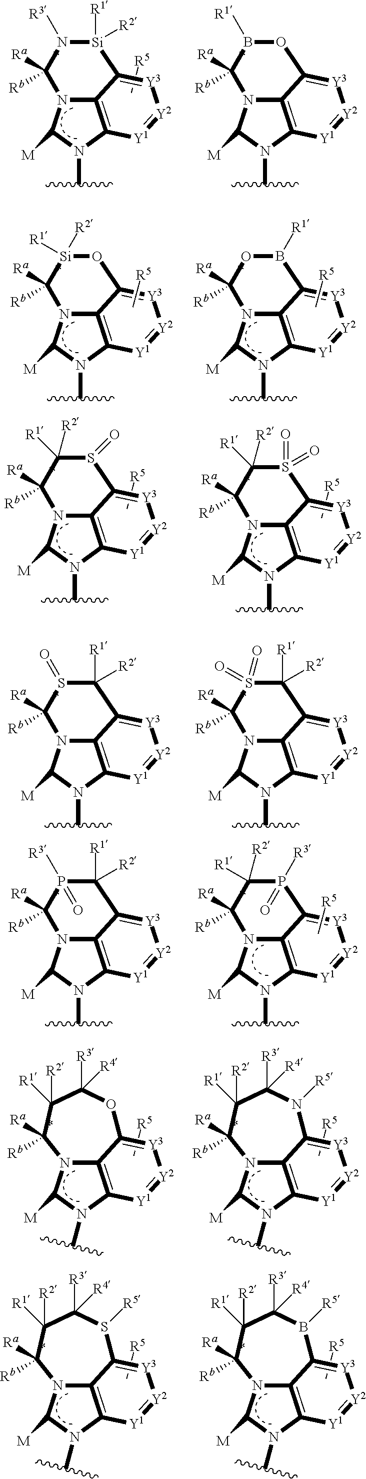

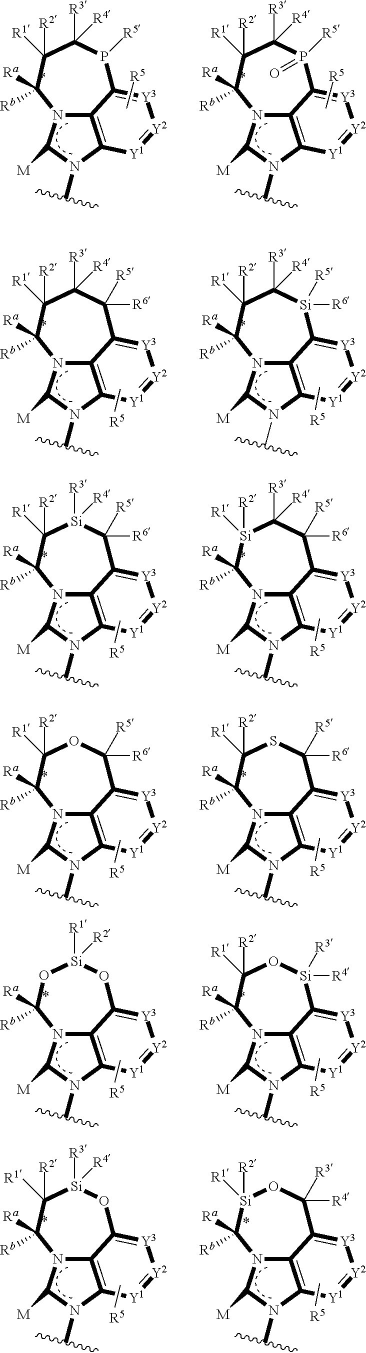

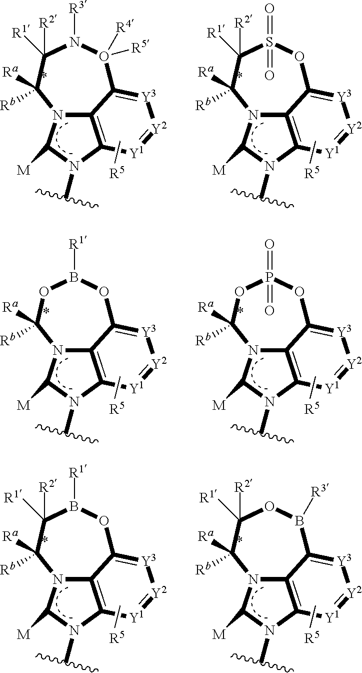

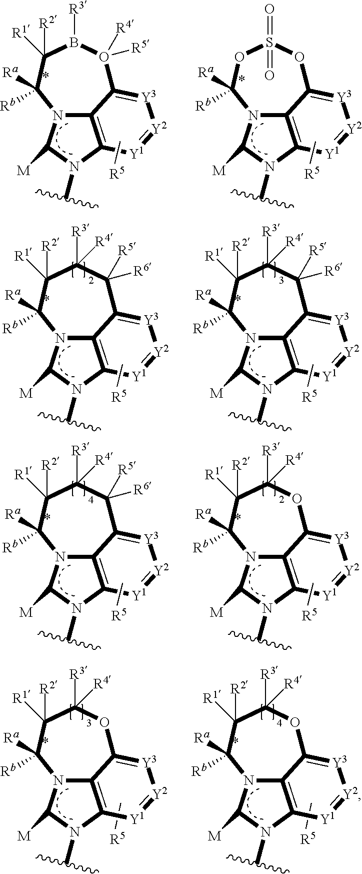

wherein, R^{1′}, R^{2′}, R^{3′}, R^{4′}, R^{5′} and R^{6′} are each independently hydrogen, deuterium, halogen, alkyl, cycloalkyl, aryl, heterocycloalkyl, heteroaryl, haloalkyl, haloaryl, haloheteroaryl, alkoxy, aryloxy, alksiliyl, arylsiliyl, heteroarylsiliyl, alkyl (hetero) arylsiliyl, alkenyl, cycloalkenyl, alkynyl, hydroxyl, mercapto, nitro, cyano, amino, mono- or dialkylamino, mono- or diarylamino, ester, isonitrile, alkoxycarbonyl, amido, alkoxycarbonylamino, aryloxycarbonylamino, sulfonylamino, sulfamoyl, carbamoyl, alkylthio, sulfinyl, ureido, phosphonamido, imino, sulfo, carboxyl, hydrazino, or combinations thereof; two or more adjacent R^{1′}, R^{2′}, R^{3′}, R^{4′}, R^{5′} and R^{6′} can selectively connect to form a fused ring.

Furthermore, the structure of L⁵ is further optimized from the following structure and its enantiomers, but not limited to them:

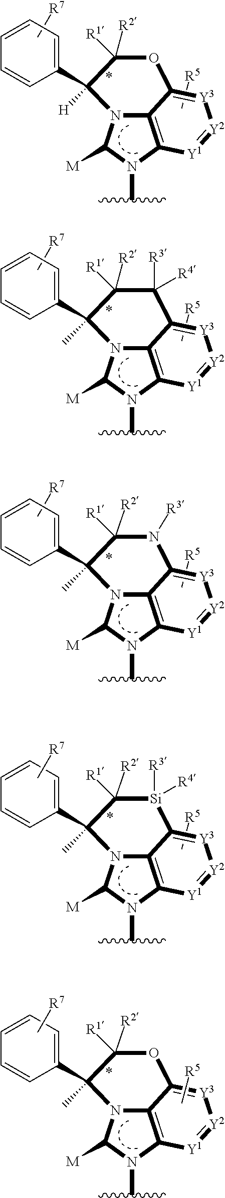

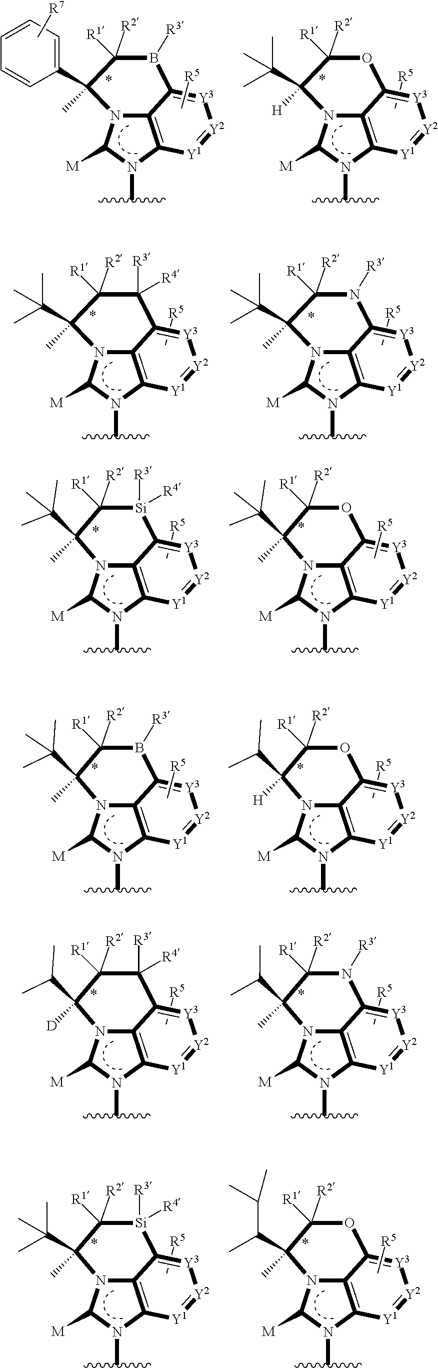

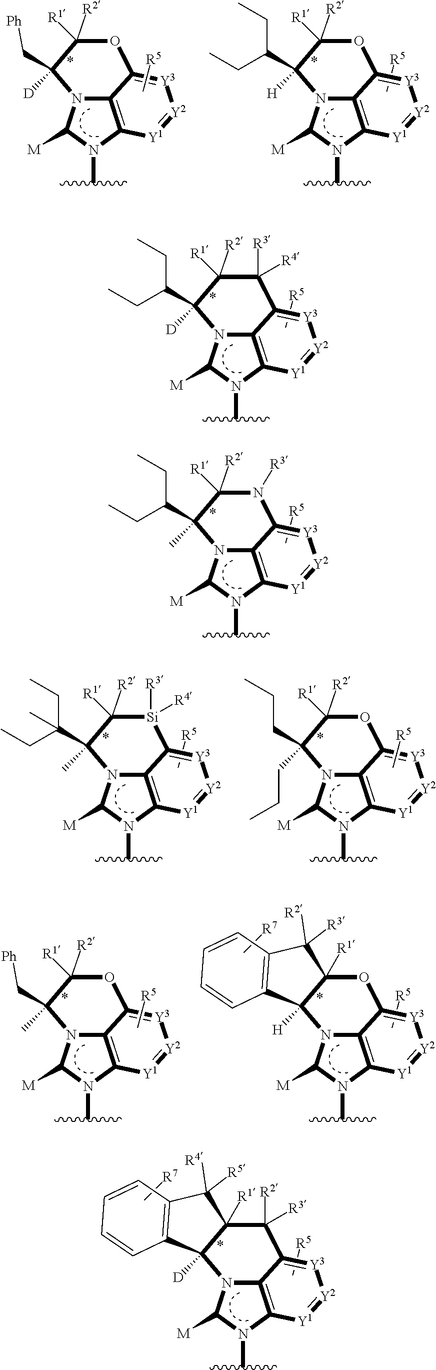

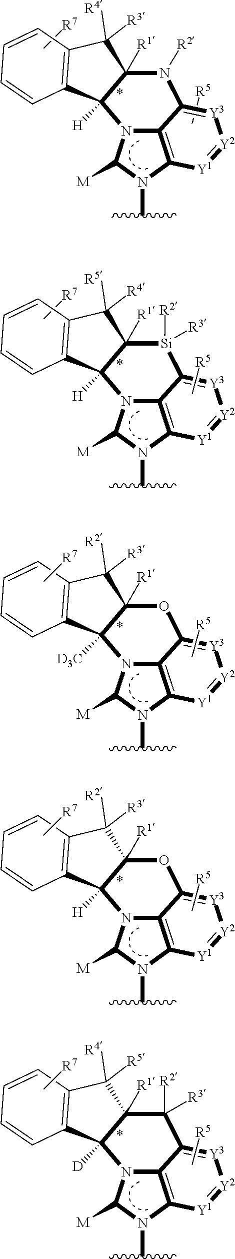

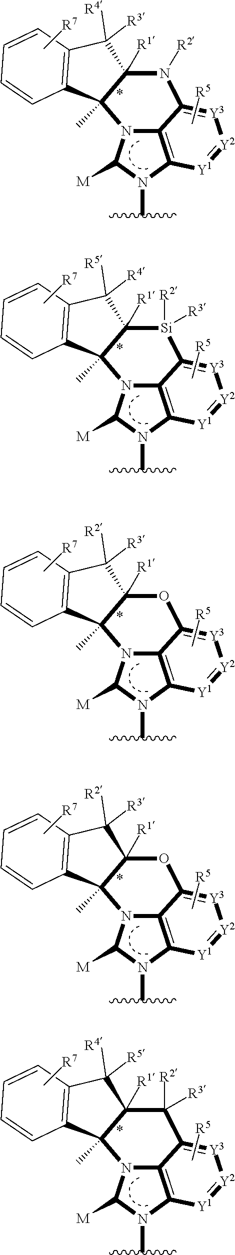

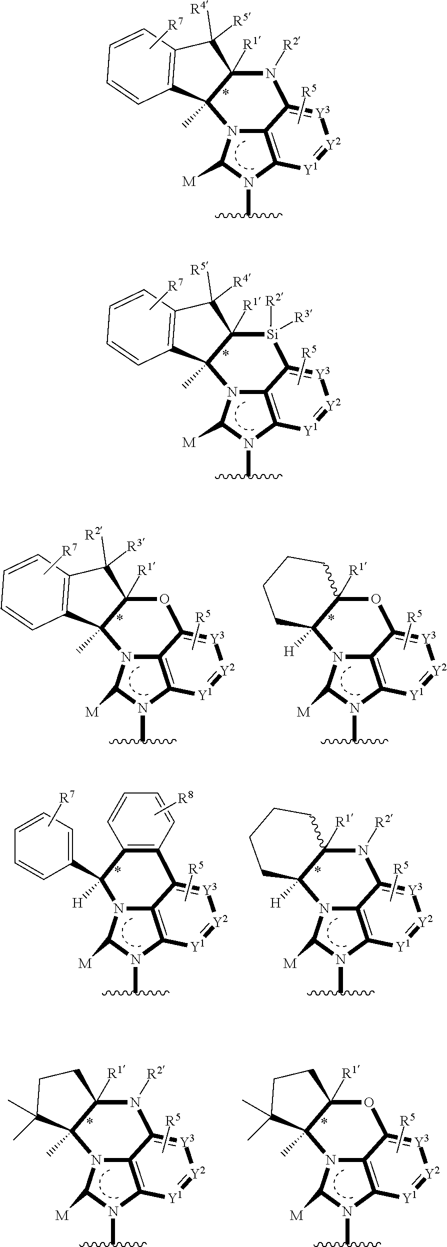

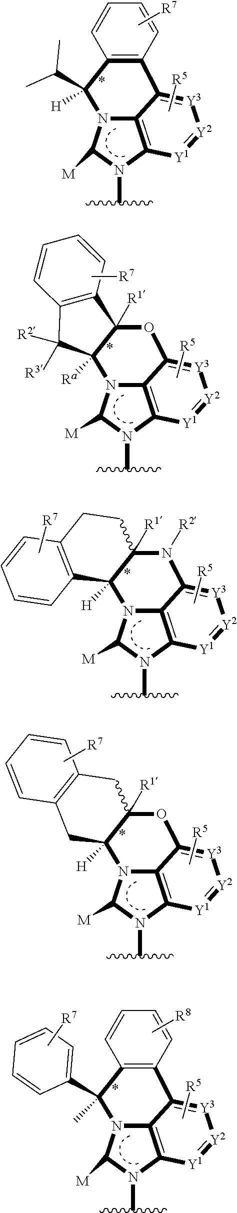

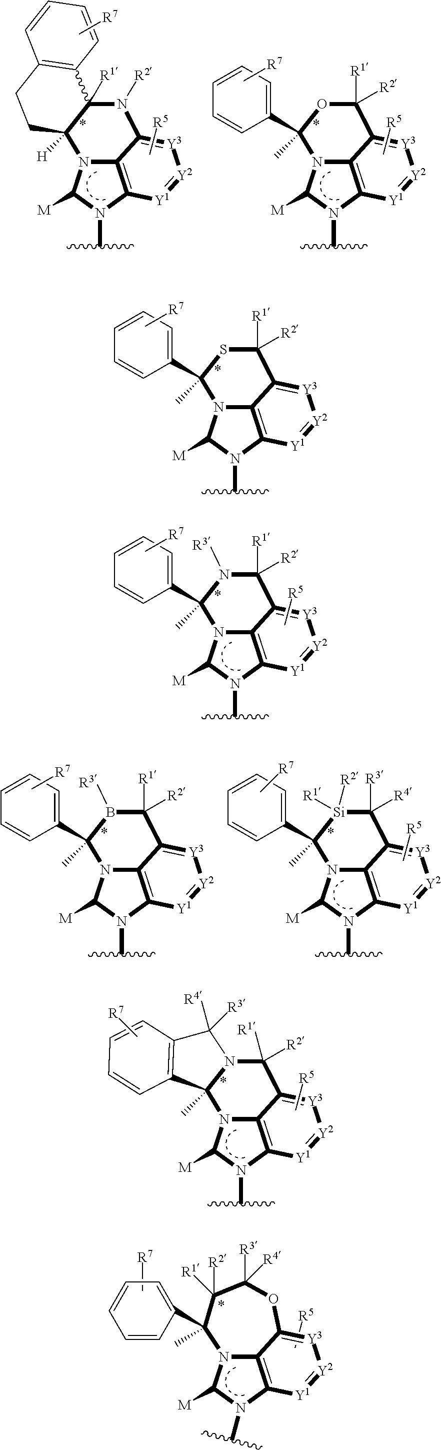

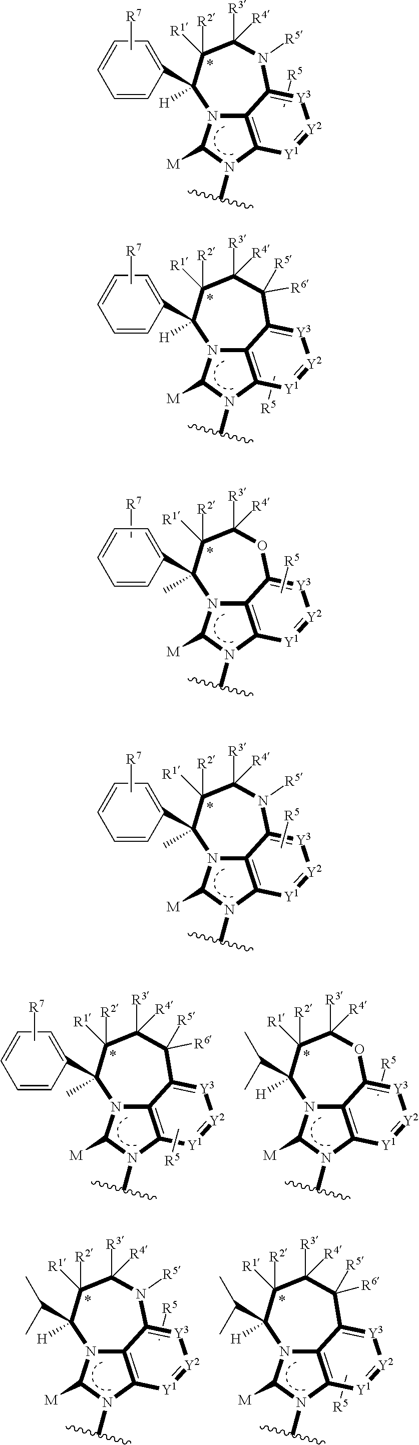

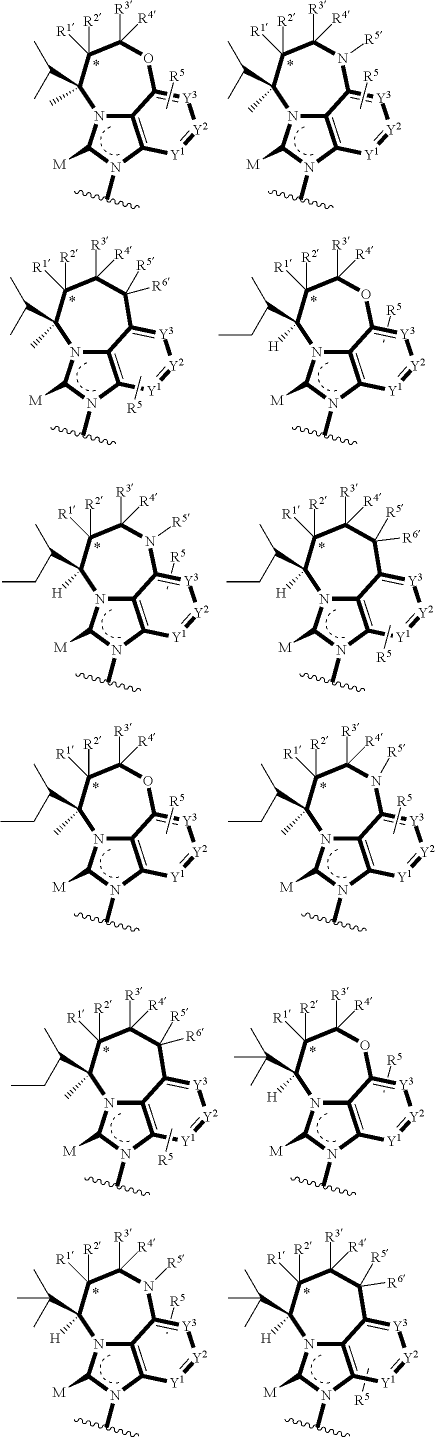

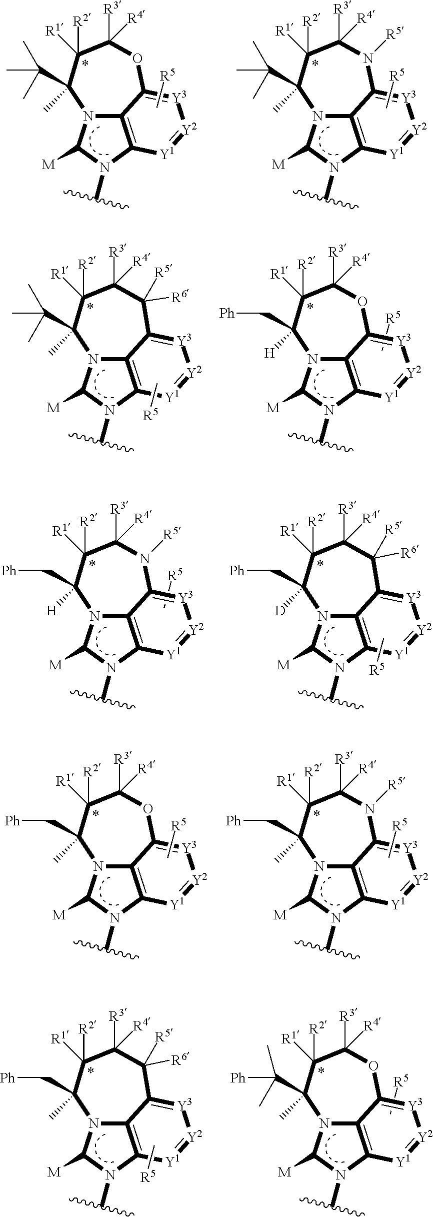

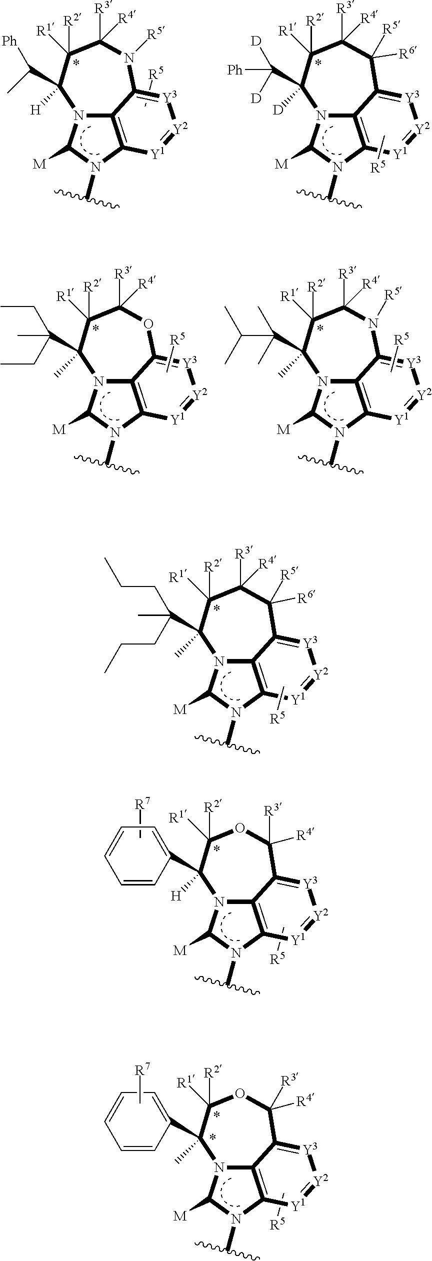

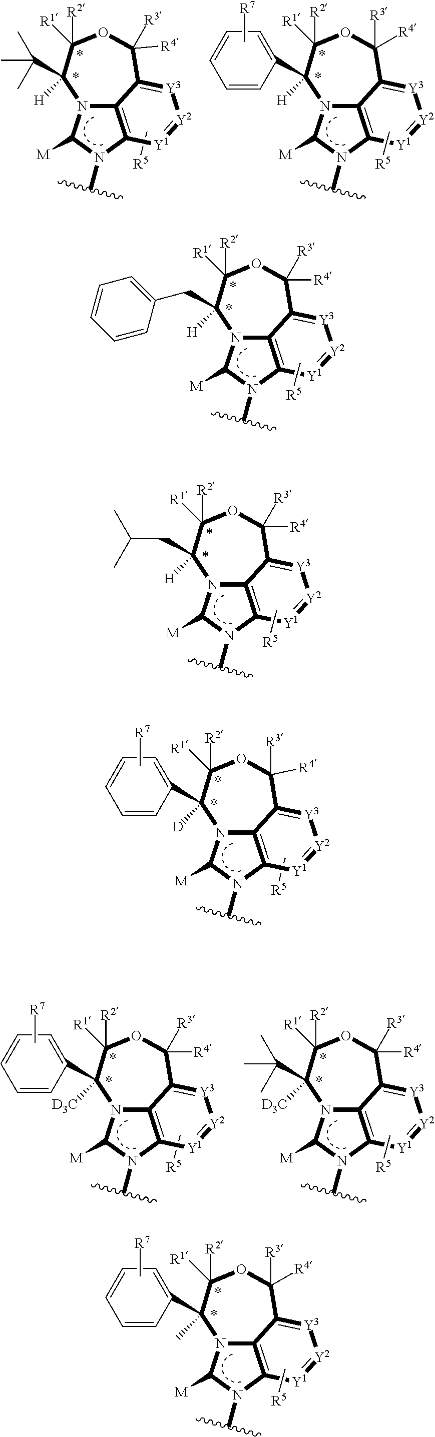

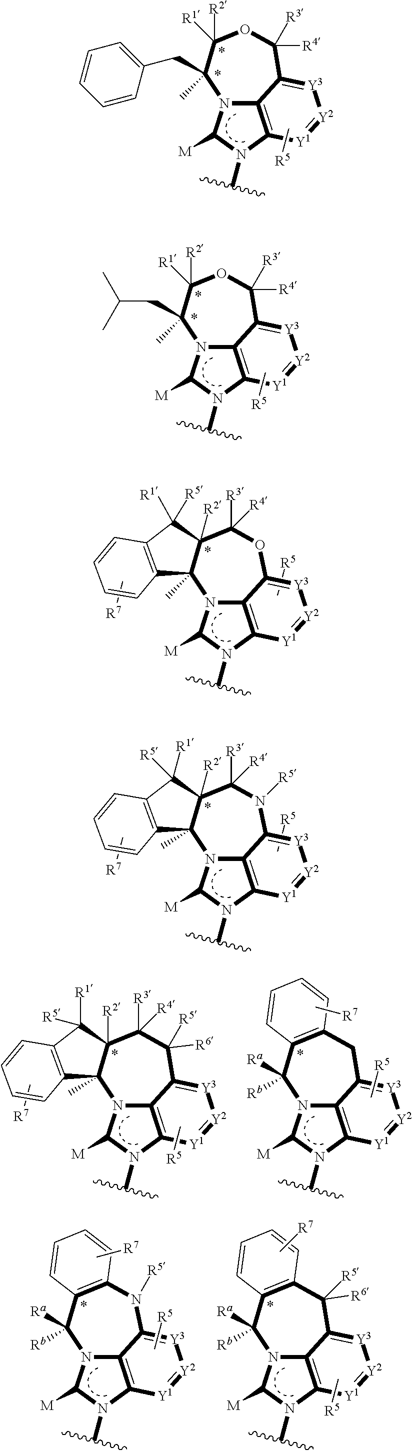

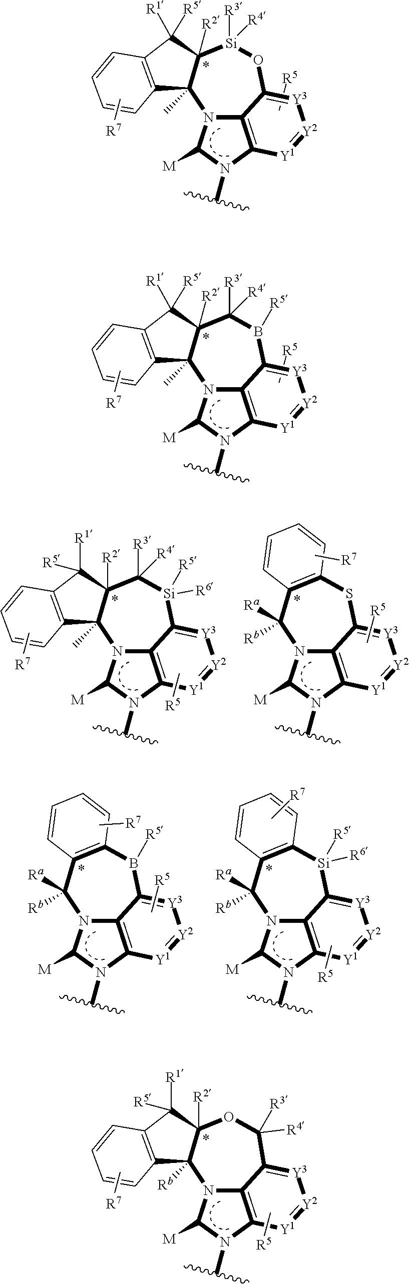

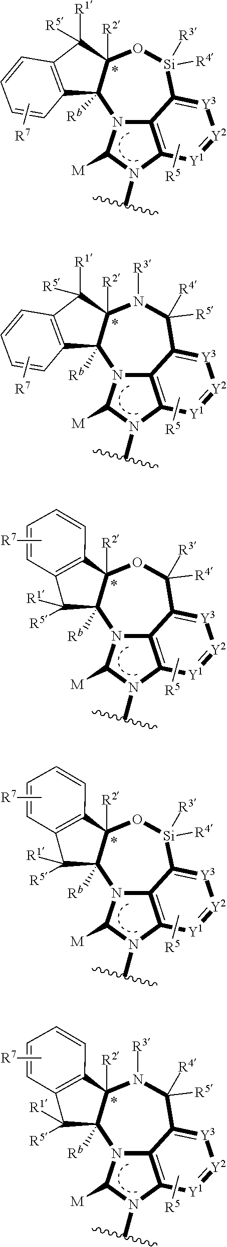

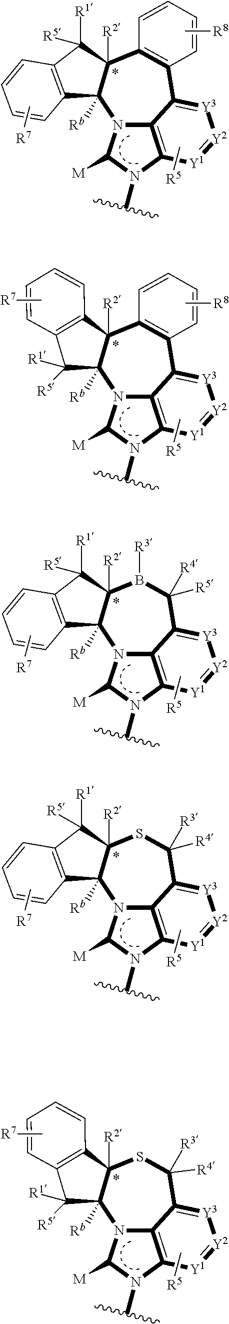

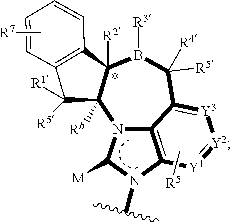

R⁷ and R⁸ each independently representing single, double, three, four, or five substituted or unsubstituted, and meanwhile, R⁷ and R⁸ are each independently hydrogen, deuterium, halogen, alkyl, cycloalkyl, aryl, heterocycloalkyl, heteroaryl, haloalkyl, haloaryl, haloheteroaryl, alkoxy, aryloxy, alksiliyl, arylsiliyl, heteroarylsiliyl, alkyl (hetero) arylsiliyl, alkenyl, cycloalkenyl, alkynyl, hydroxyl, mercapto, nitro, cyano, amino, mono- or dialkylamino, mono- or diarylamino, ester, isonitrile, alkoxycarbonyl, amido, alkoxycarbonyl amino, aryl oxycarbonyl amino, sulfonylamino, sulfamoyl, carbamoyl, alkylthio, sulfinyl, ureido, phosphonamido, imino, sulfo, carboxyl, hydrazino, or combinations thereof; two or more adjacent R⁷ and R⁸ can selectively connect to form a fused ring; any two groups in R⁷ and R⁸ can be connected to form a cyclic system; among them, alkyl(hetero)arylsilyl is alkarylsilyl or alkyl hetero arylsilyl.

Furthermore, the helically chiral tetradentate cyclometalated platinum(II) and palladium(II) complexes circularly polarized photoluminescence materials based on the structural units of benzocarbenes and their derivatives are preferred as shown in the P-type structure (M=Pt or Pd) and its enantiomer M-type isomers in the following structures, but not limited to them:

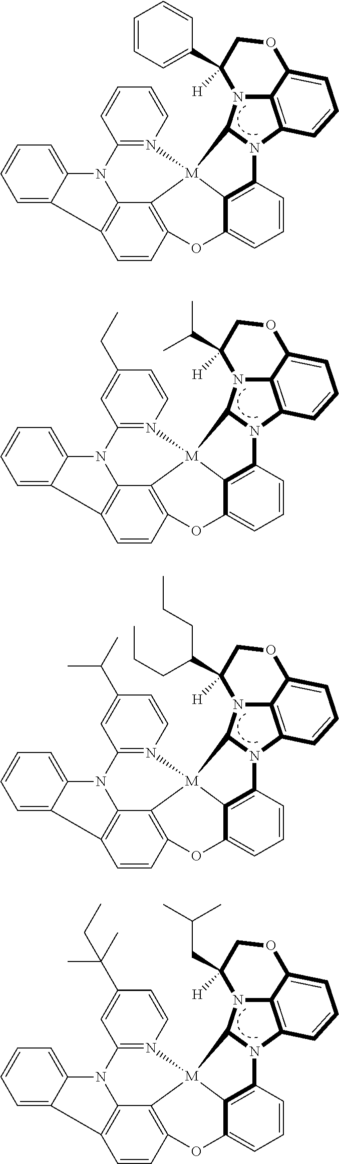

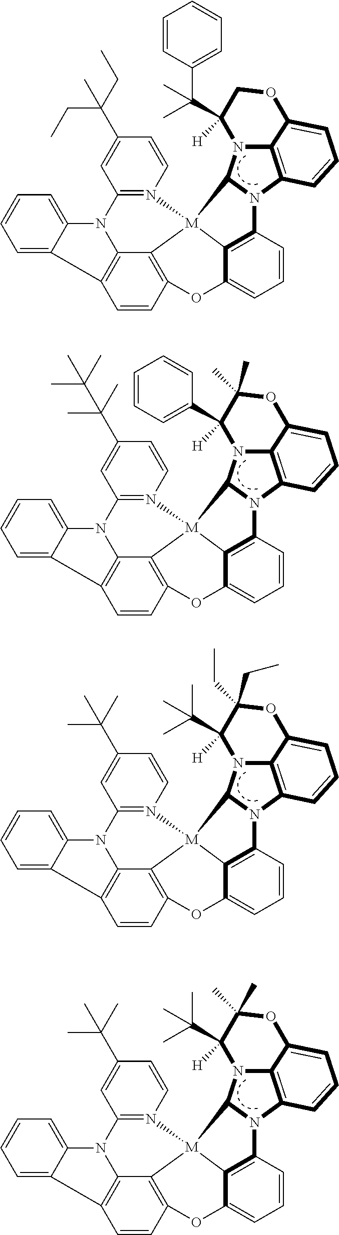

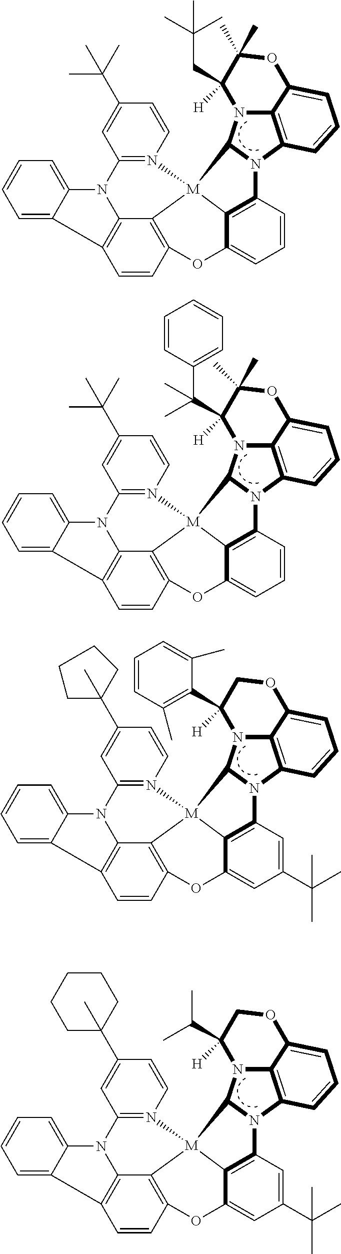

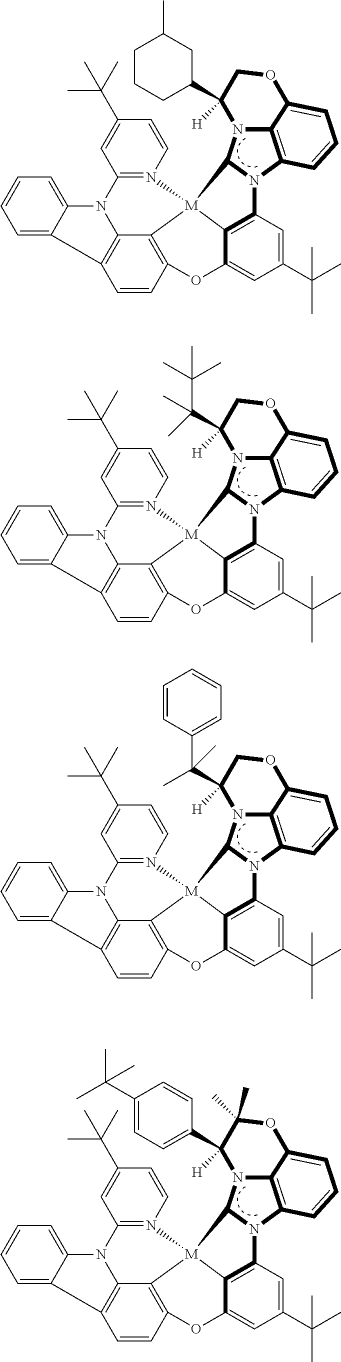

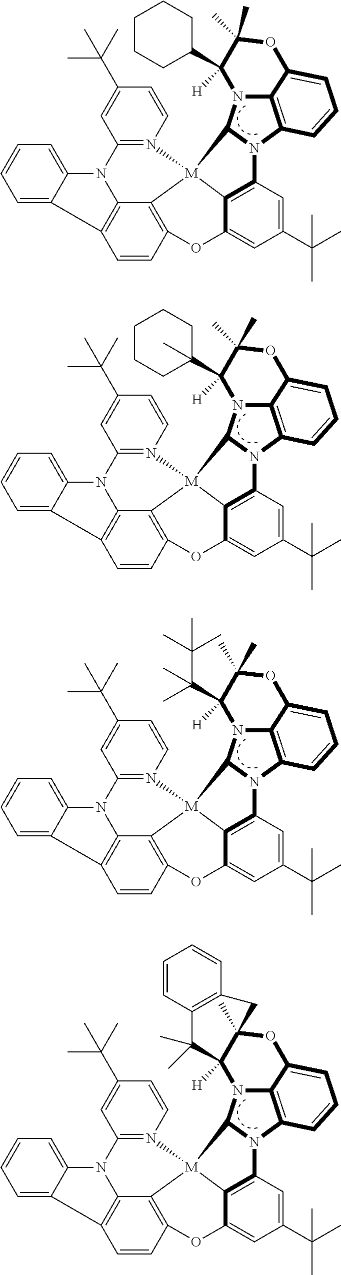

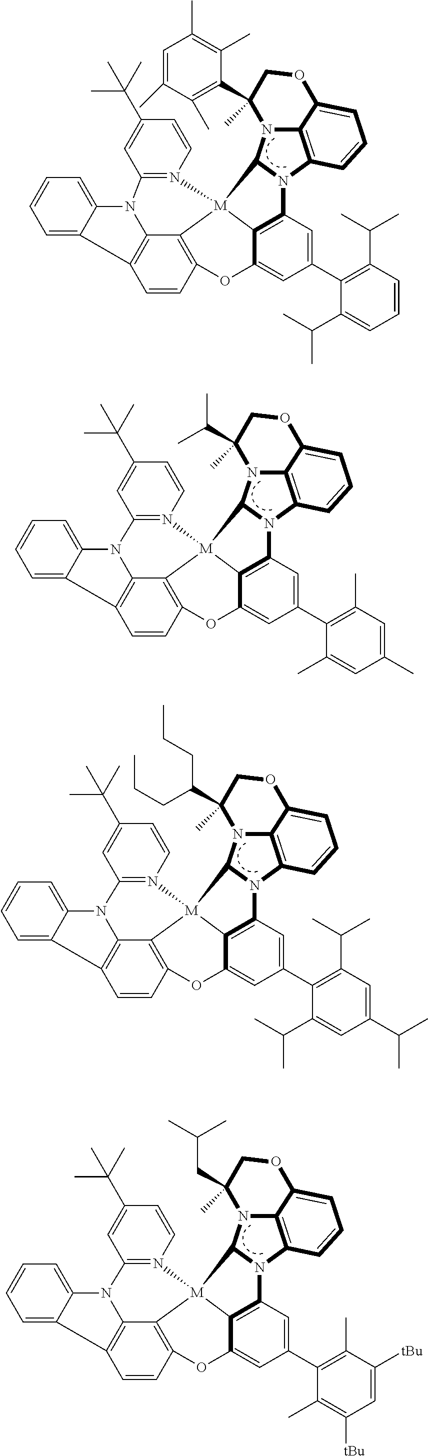

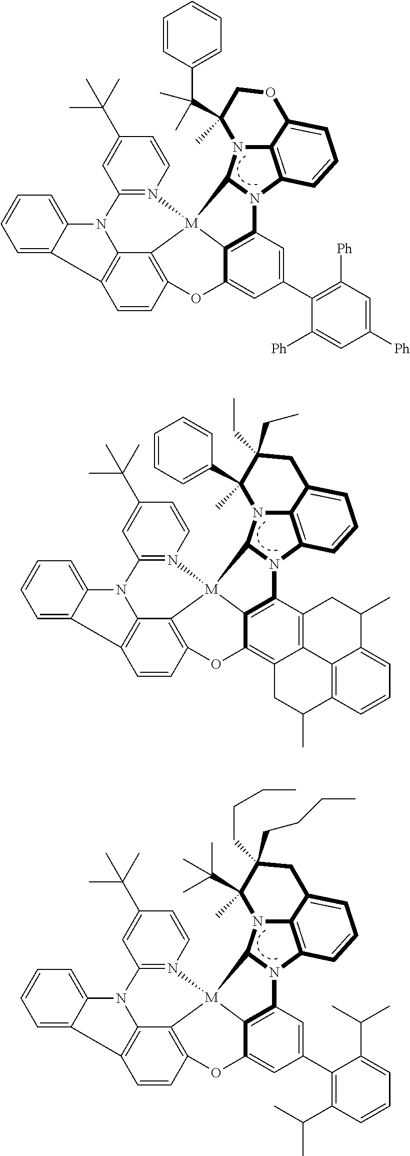

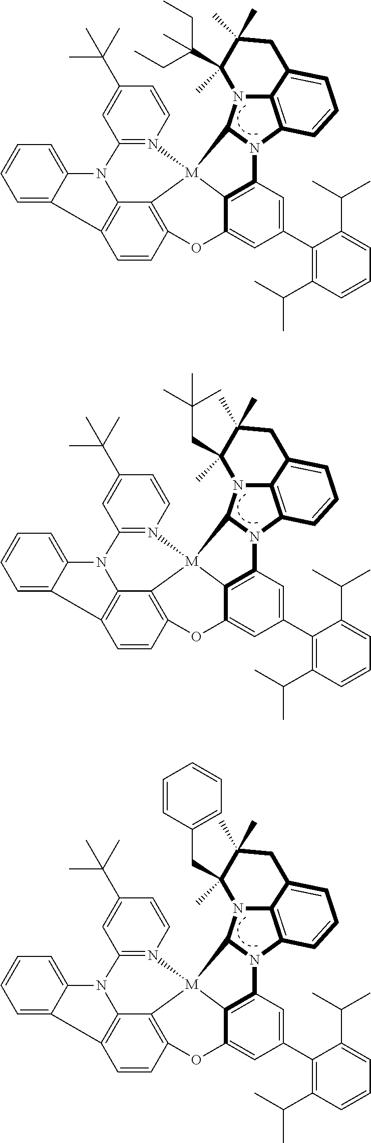

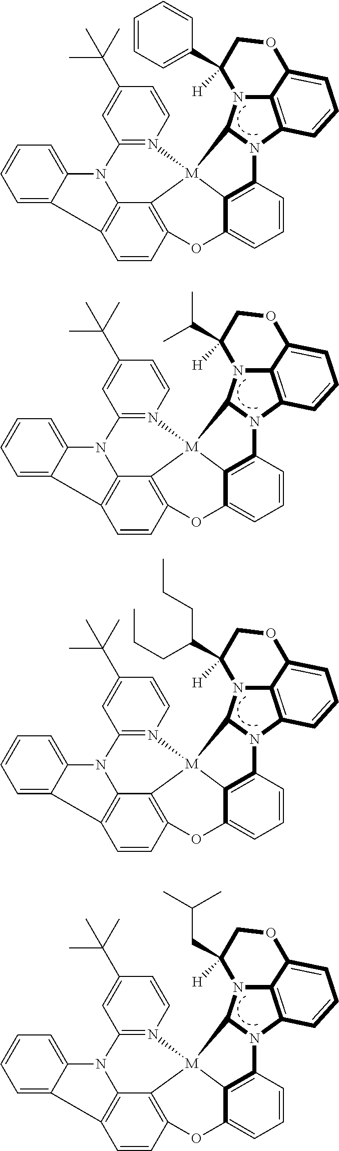

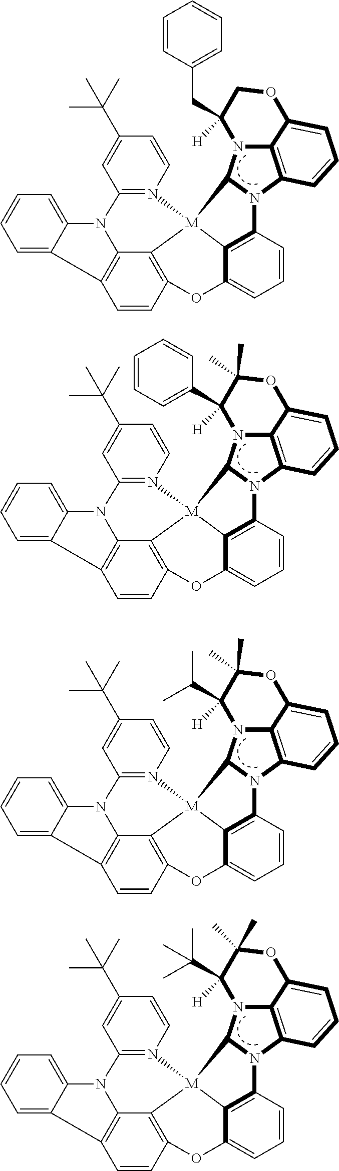

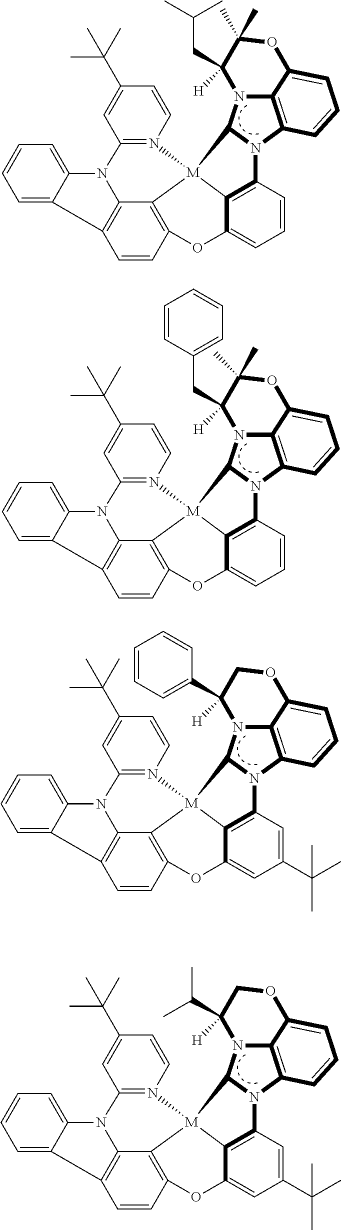

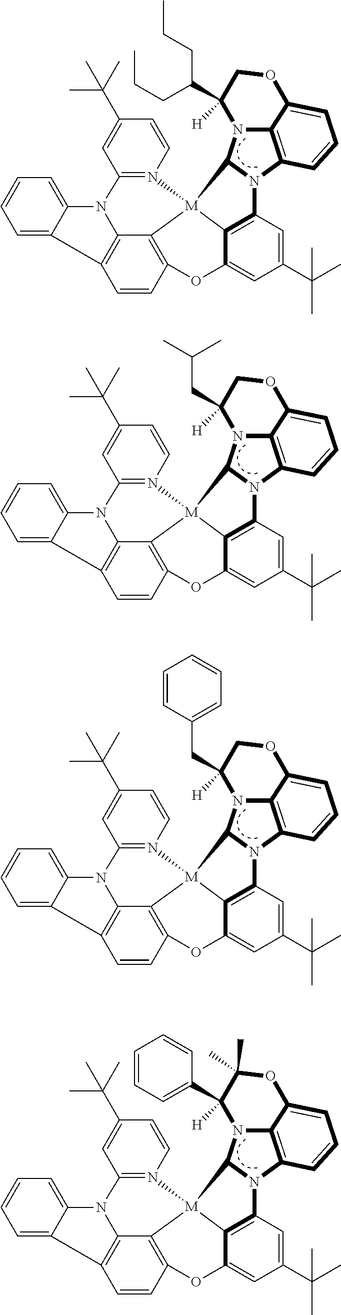

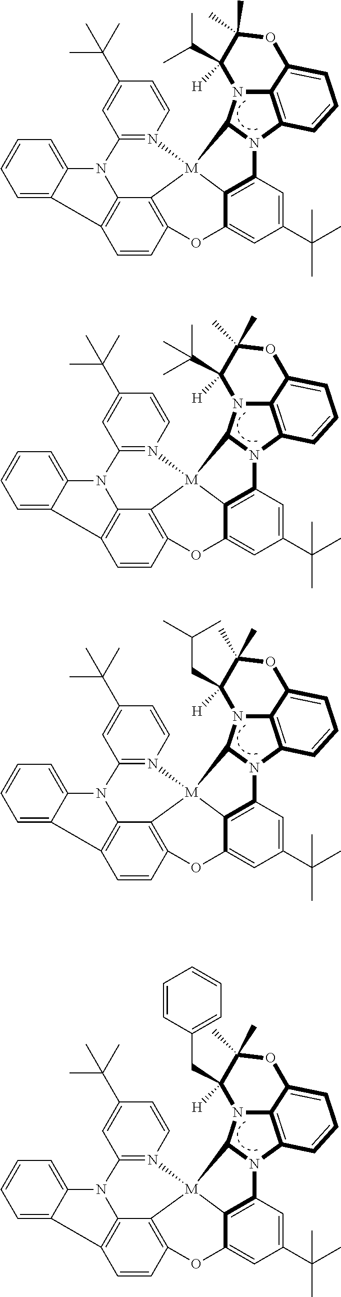

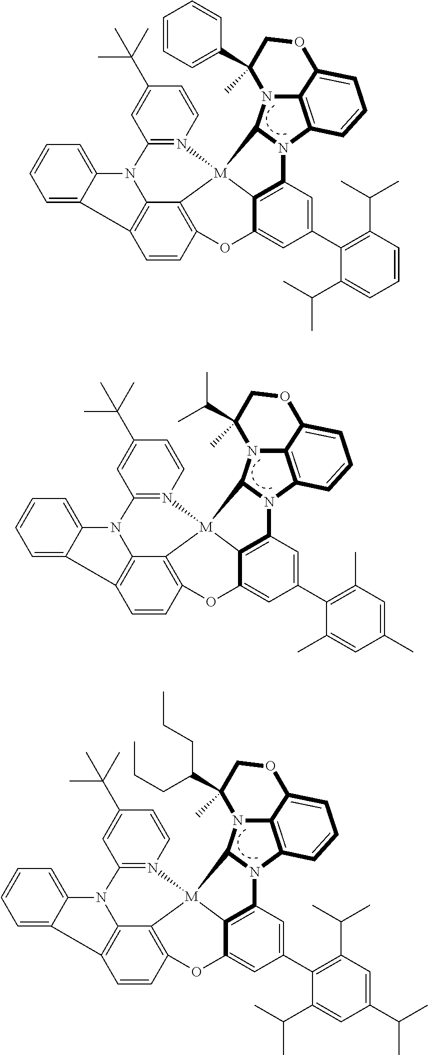

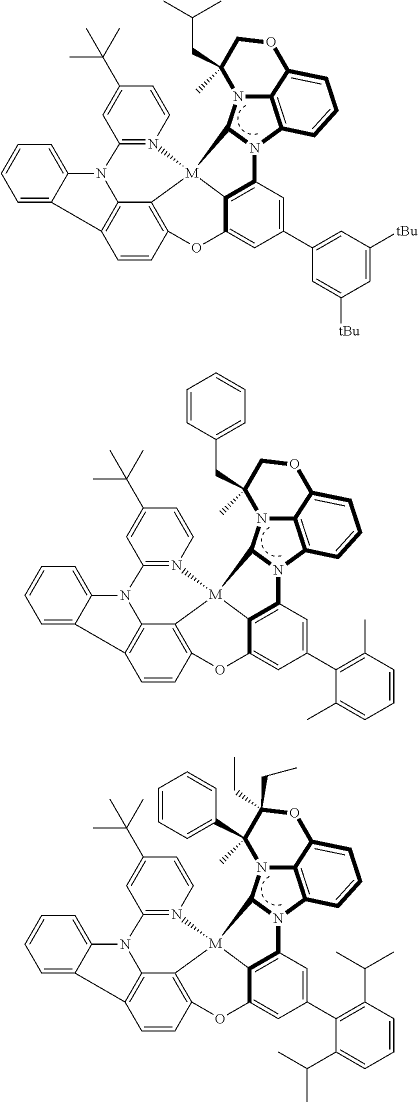

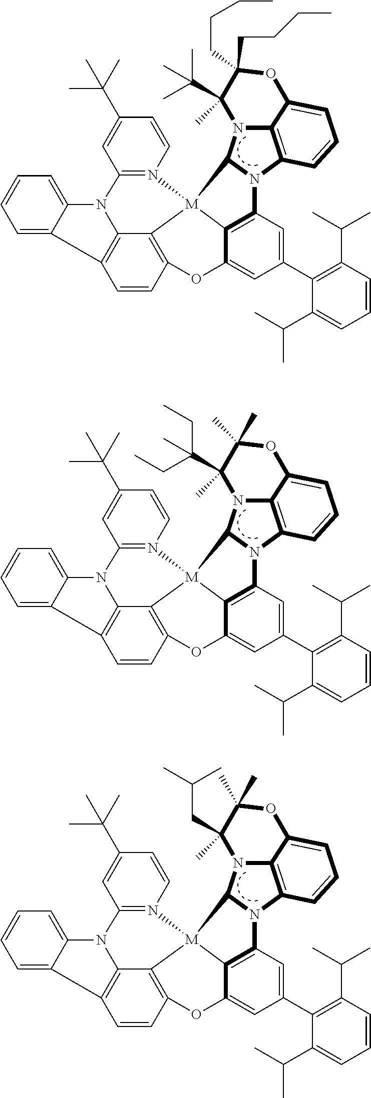

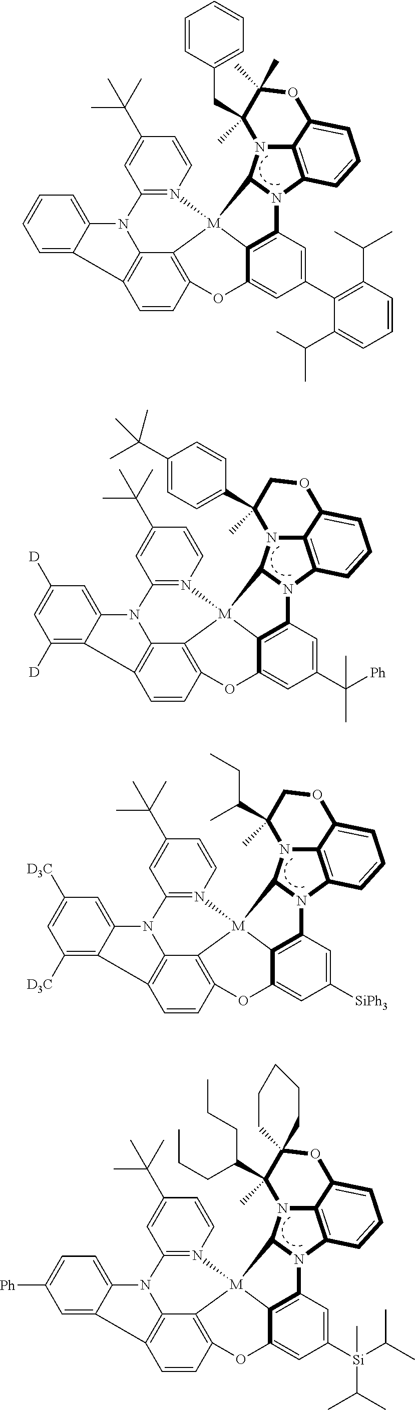

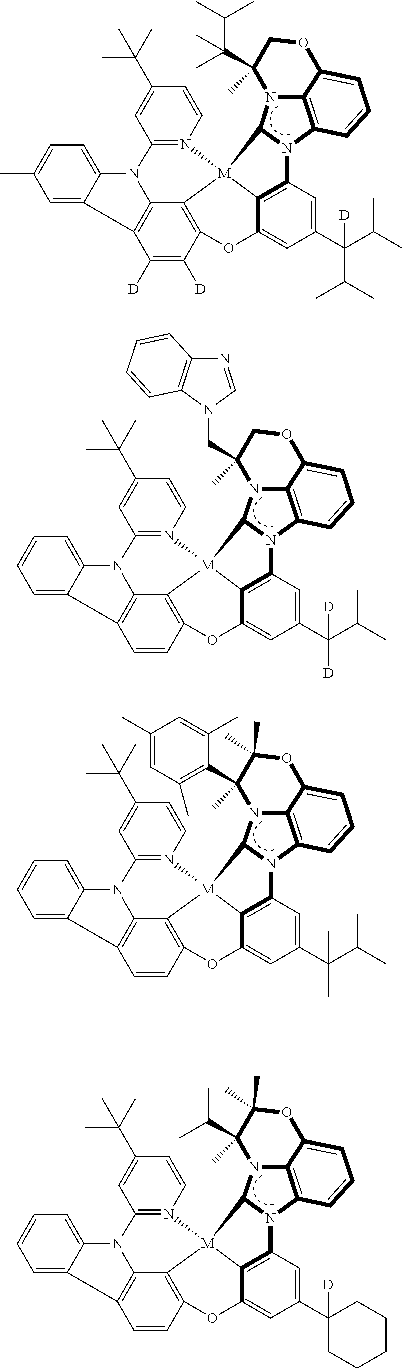

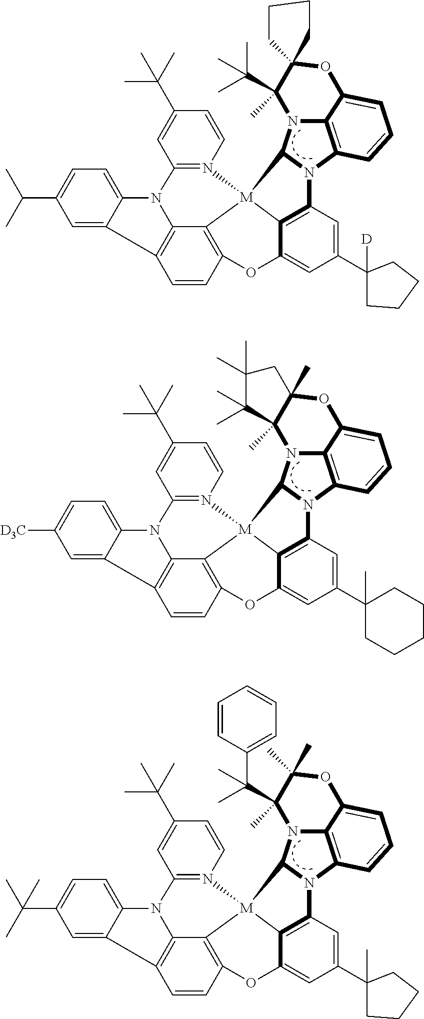

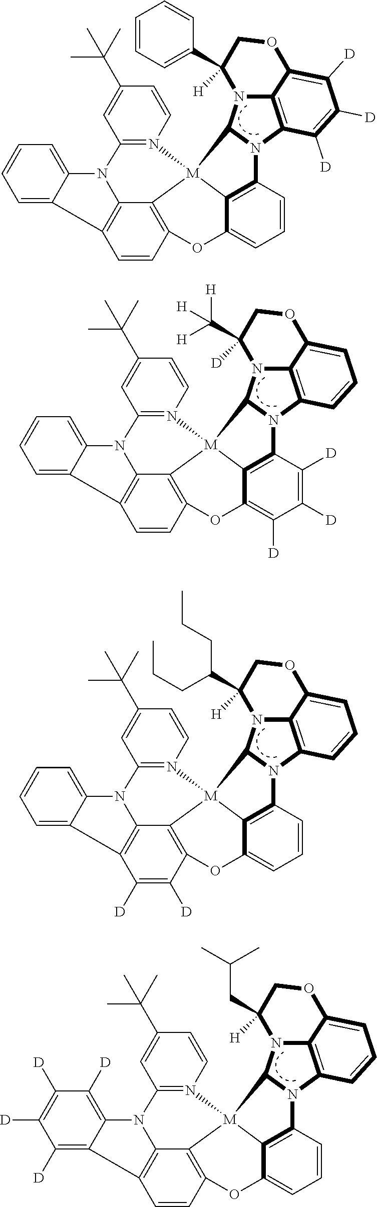

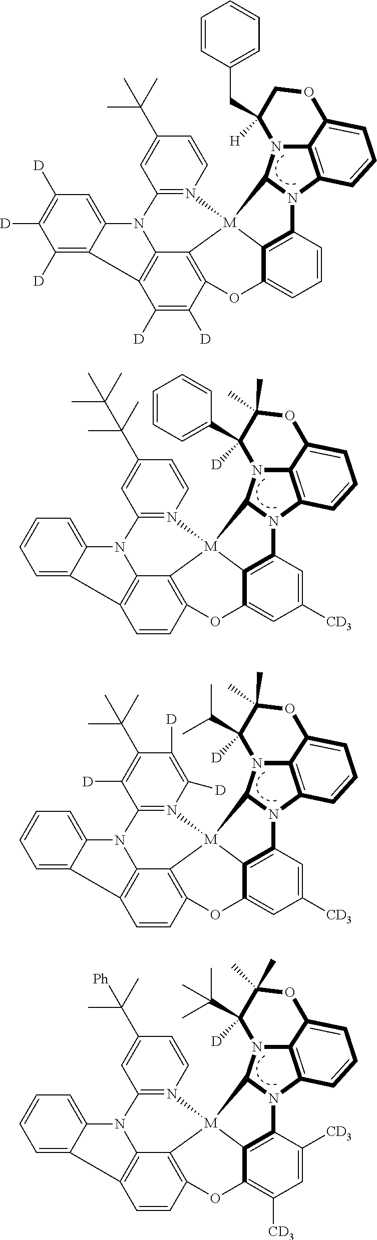

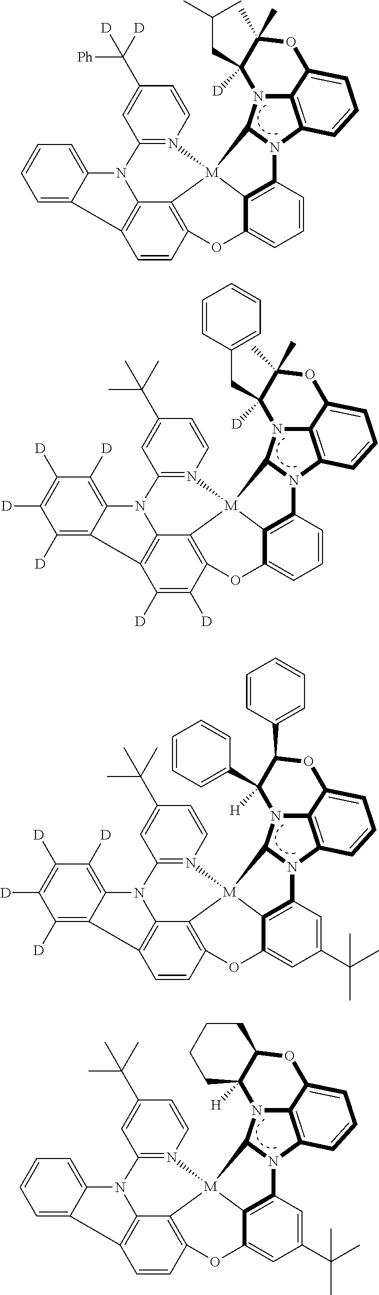

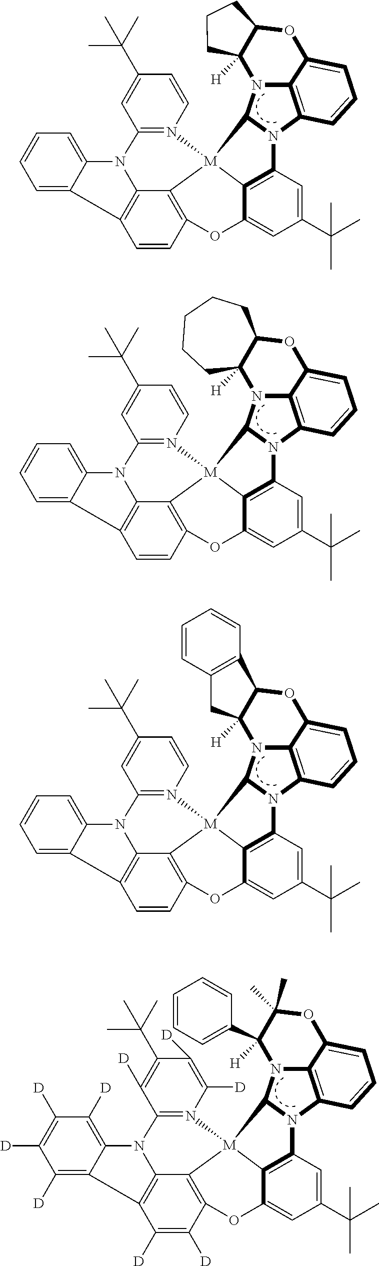

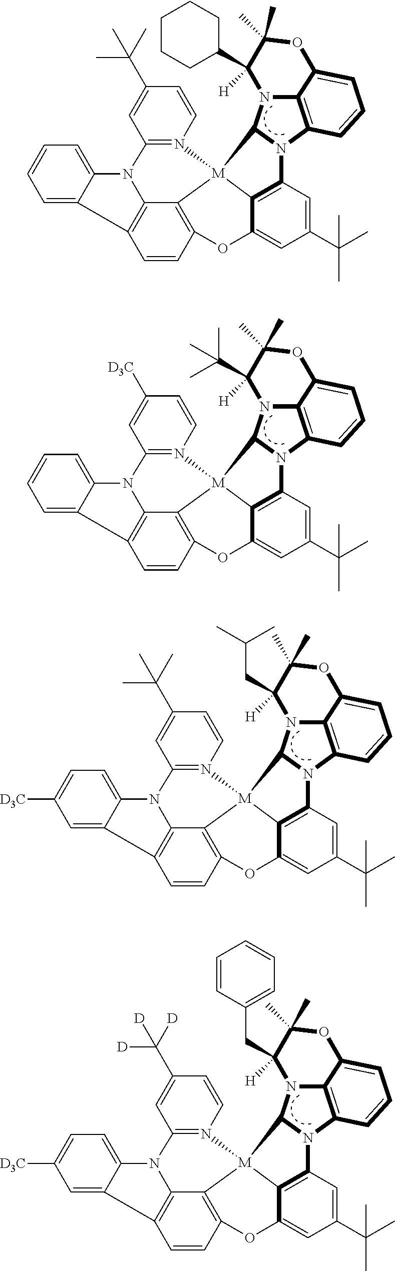

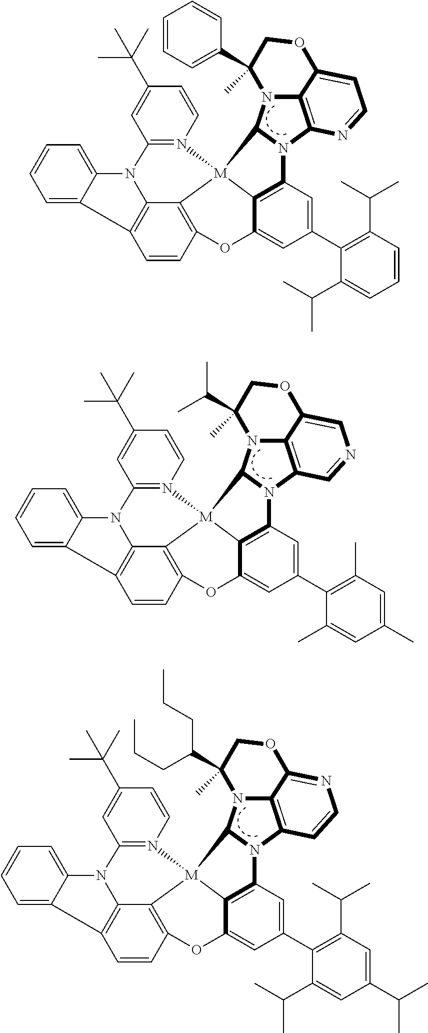

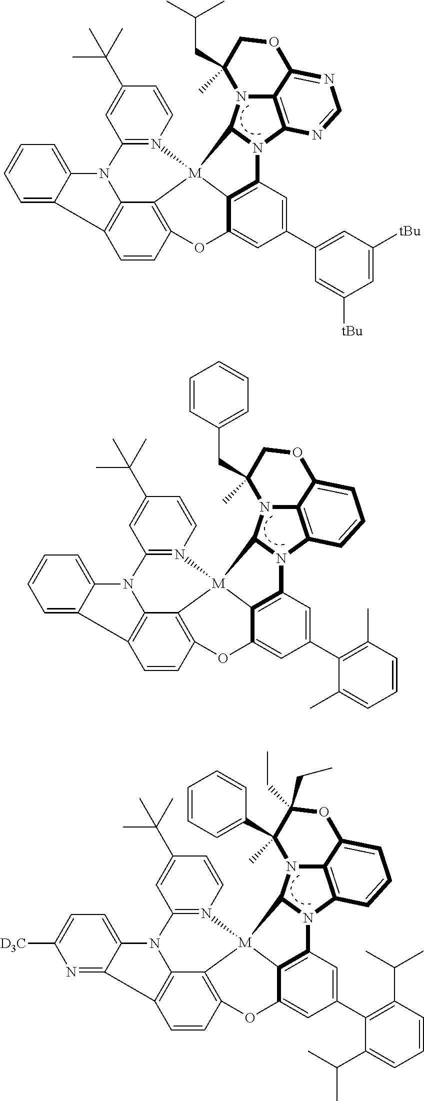

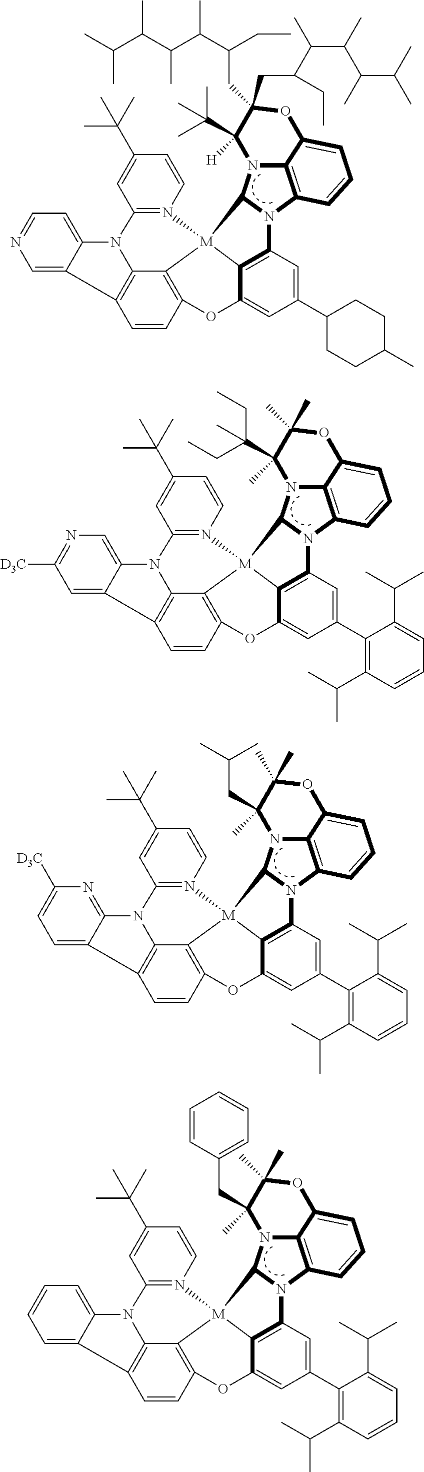

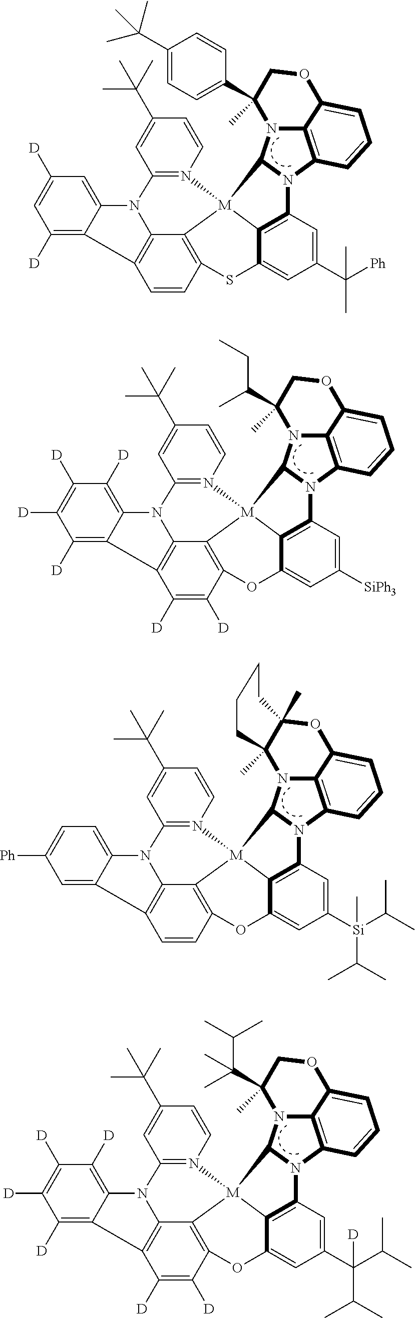

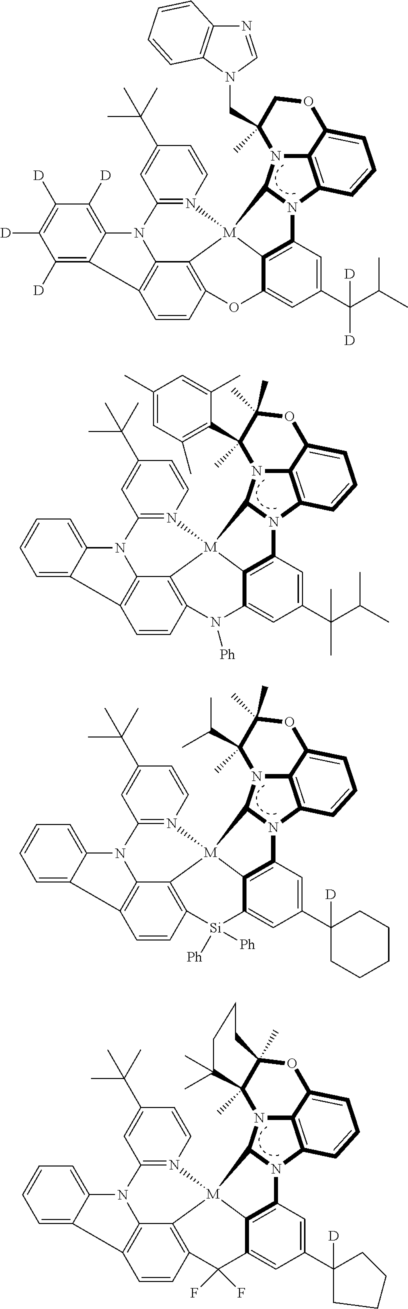

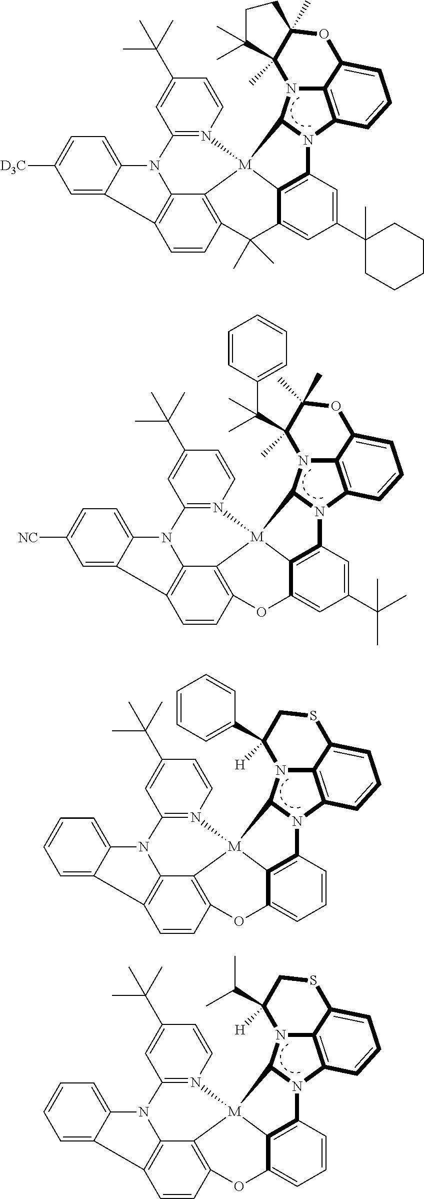

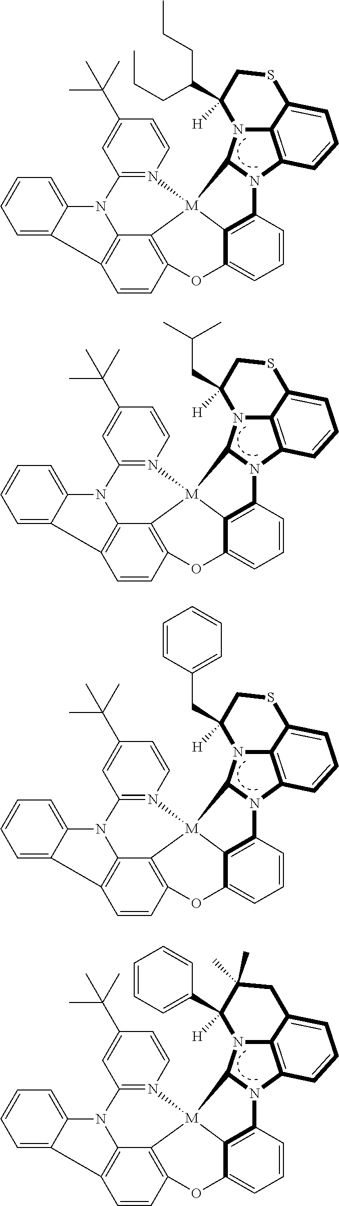

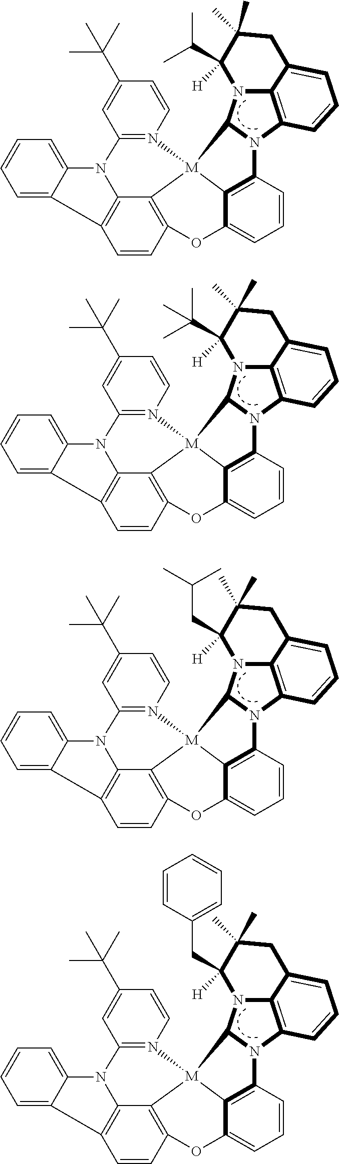

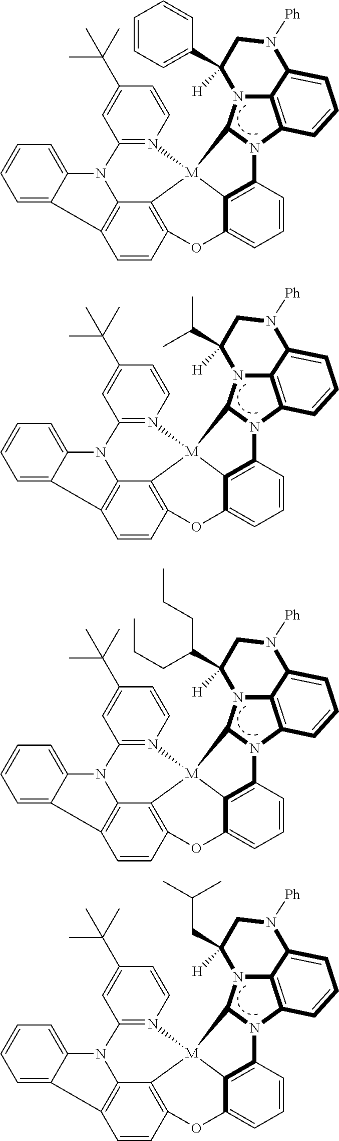

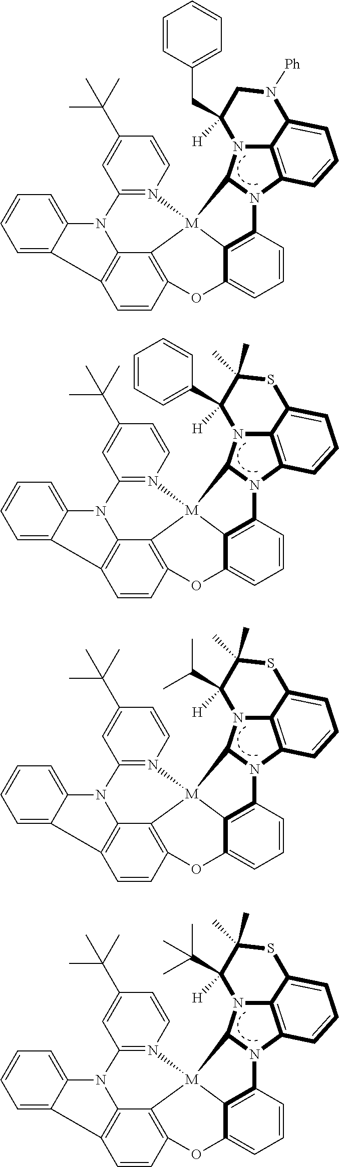

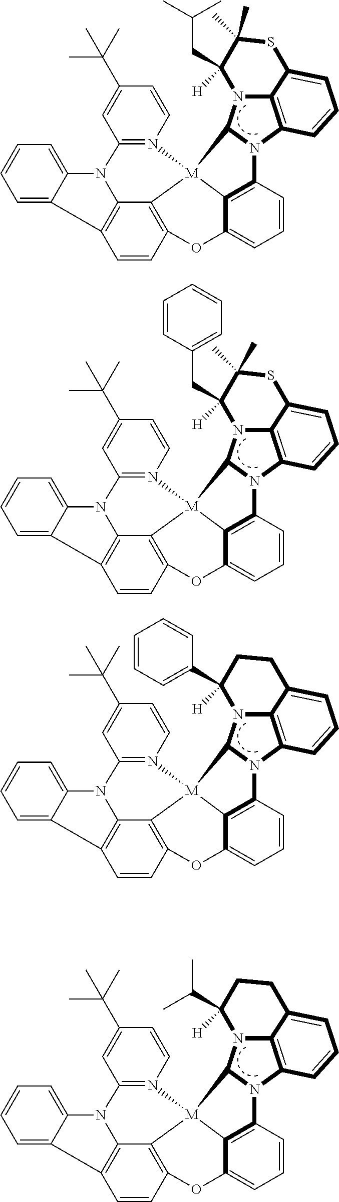

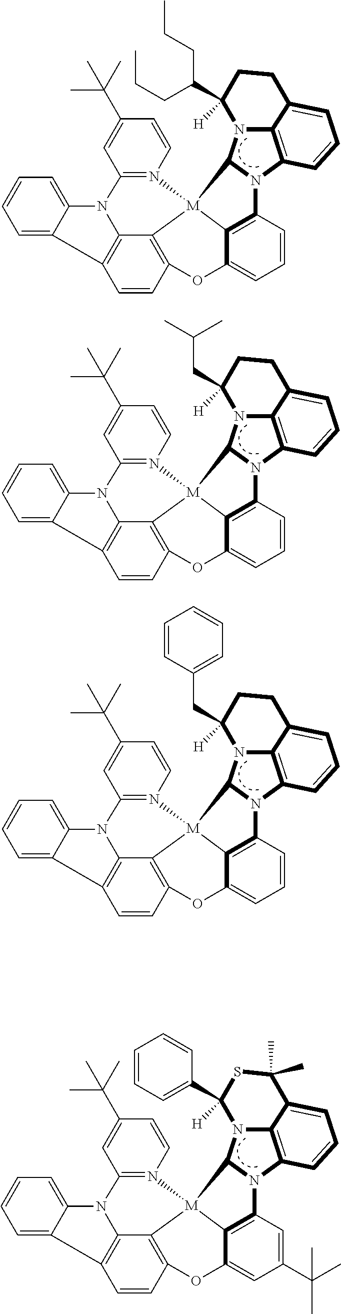

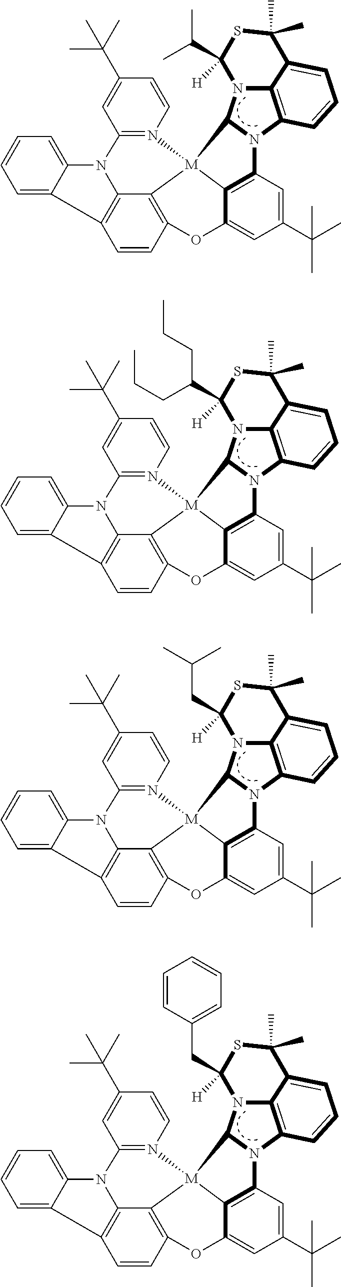

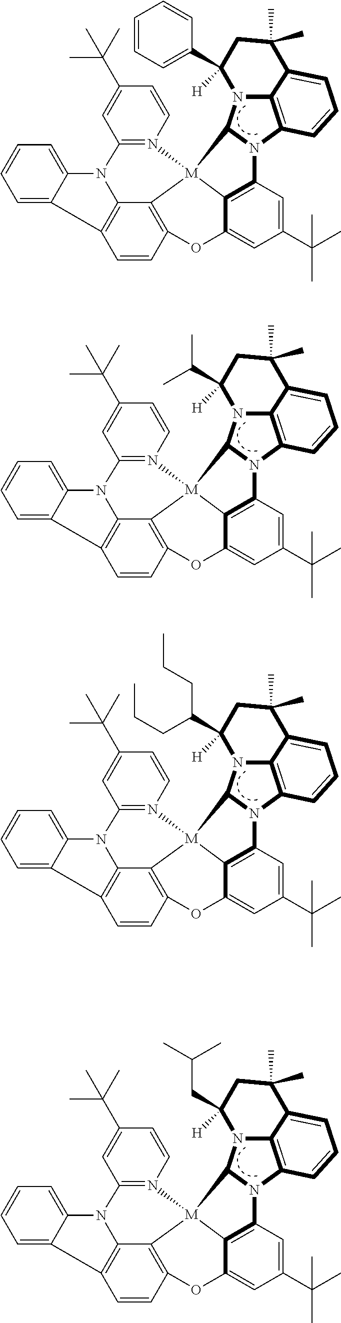

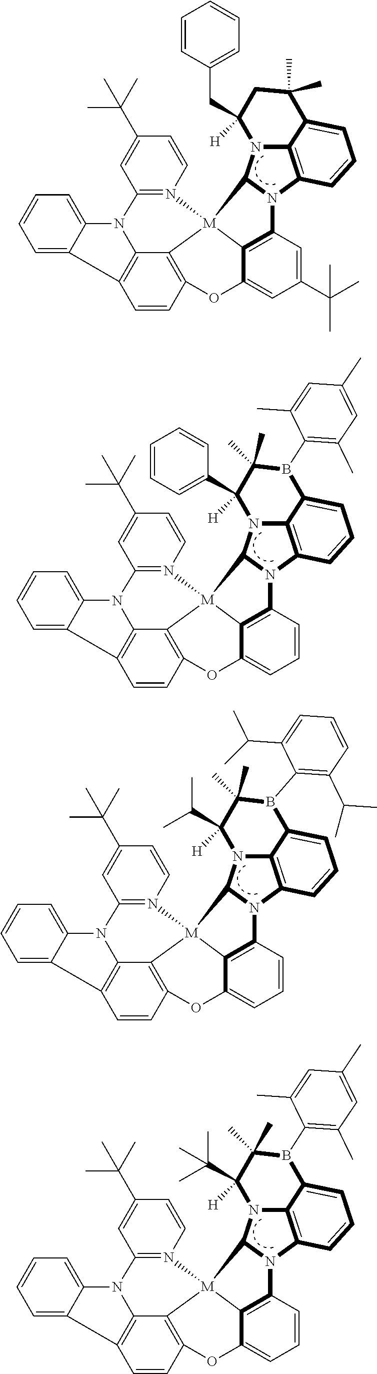

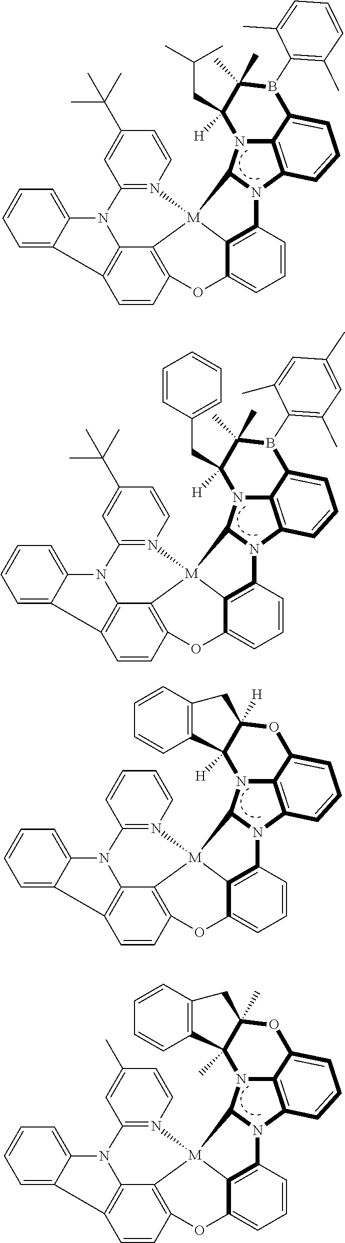

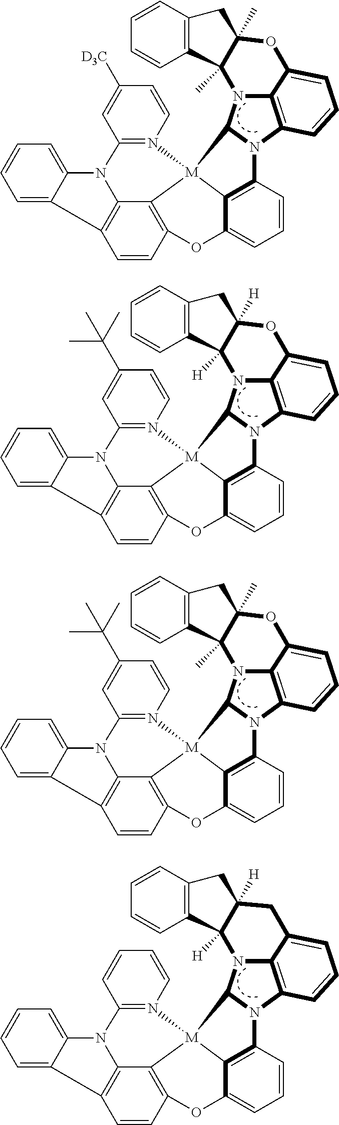

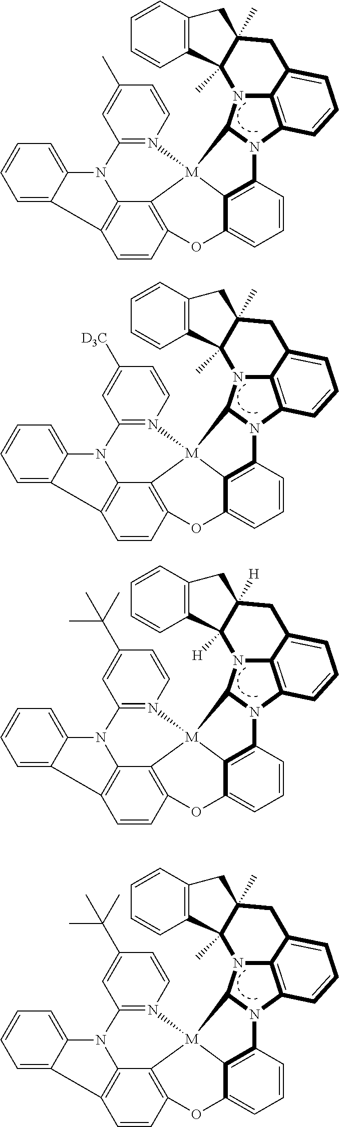

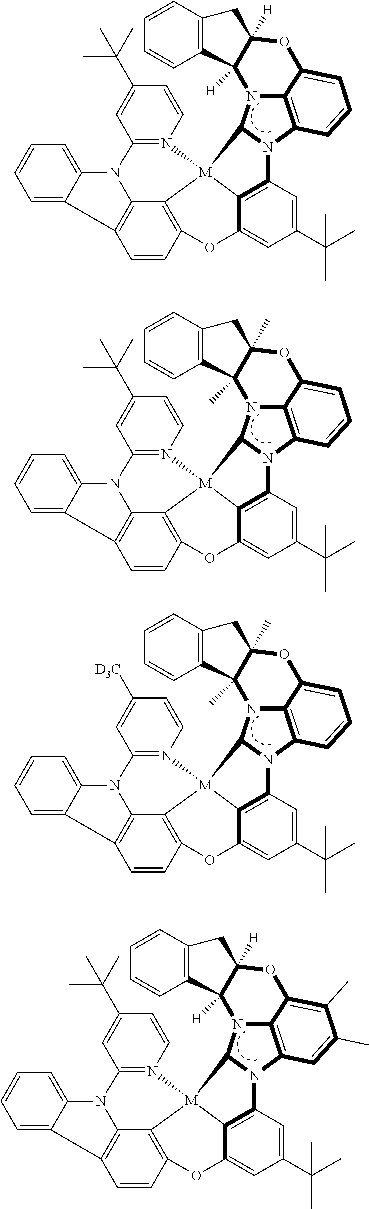

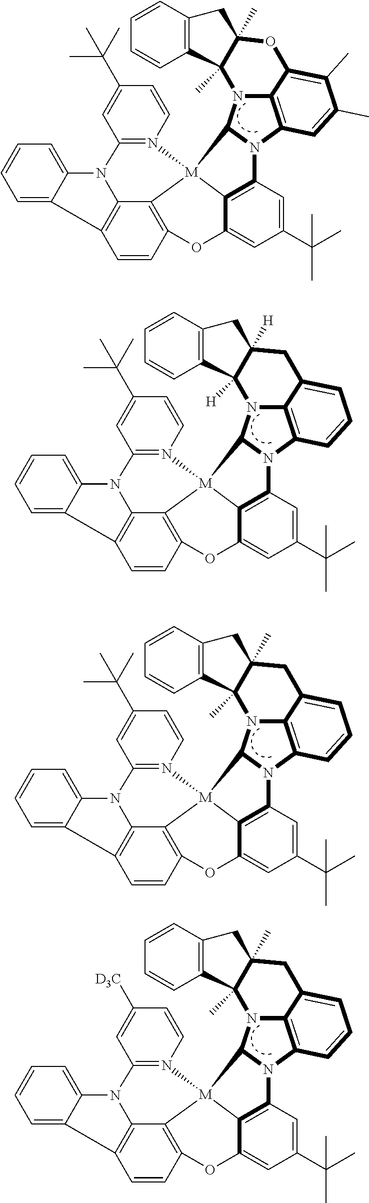

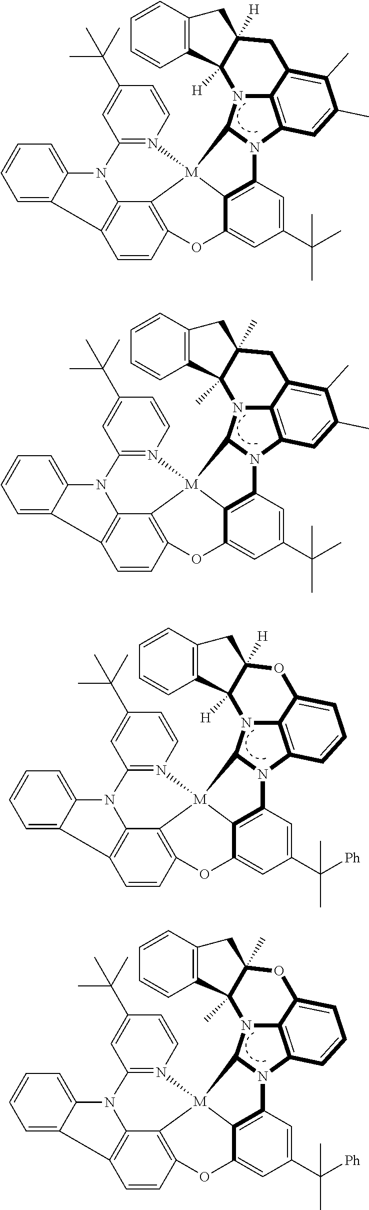

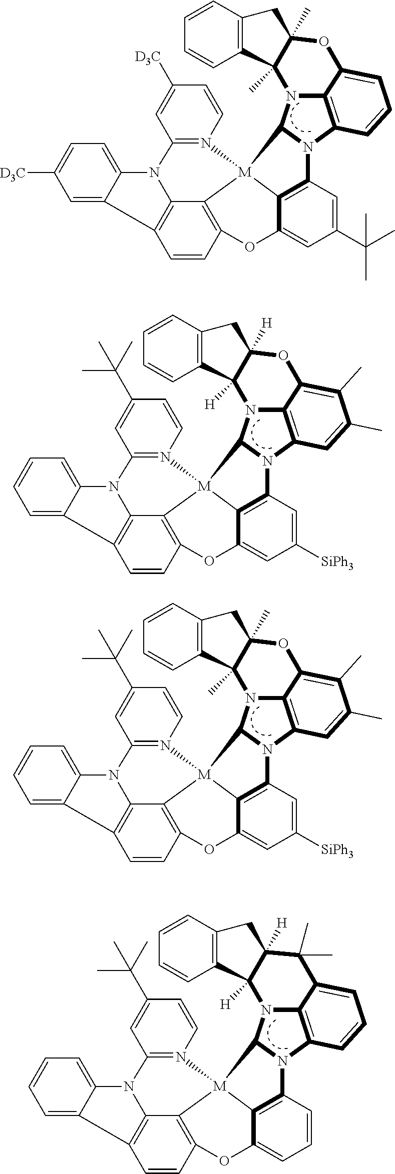

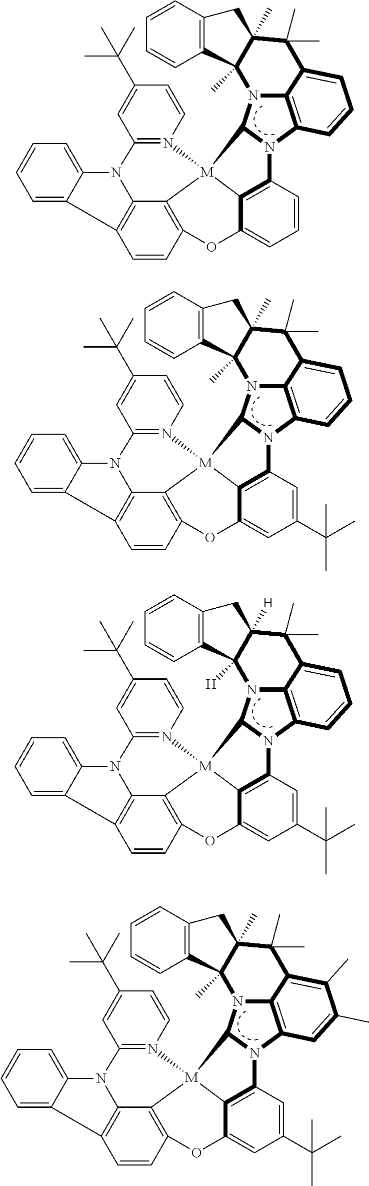

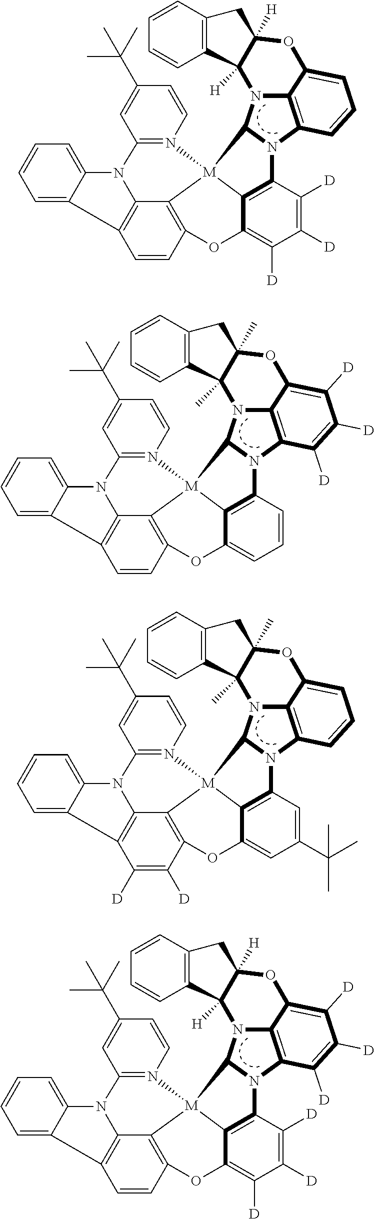

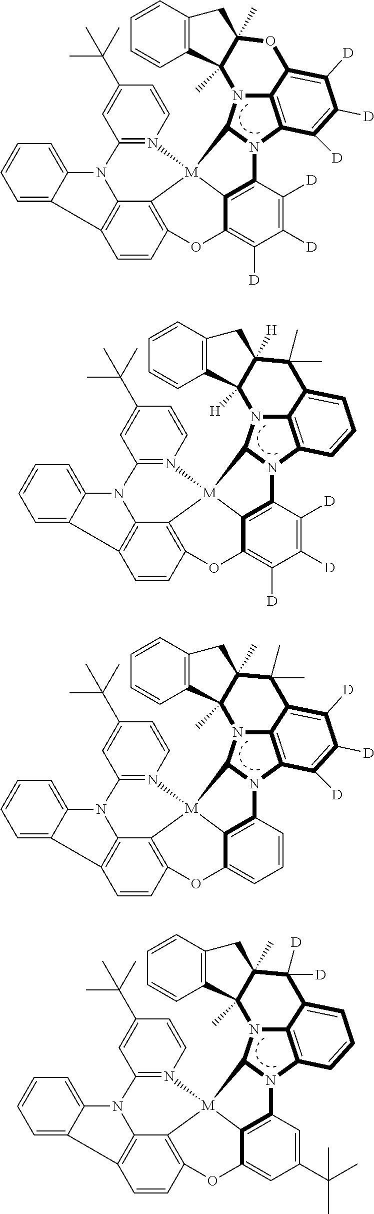

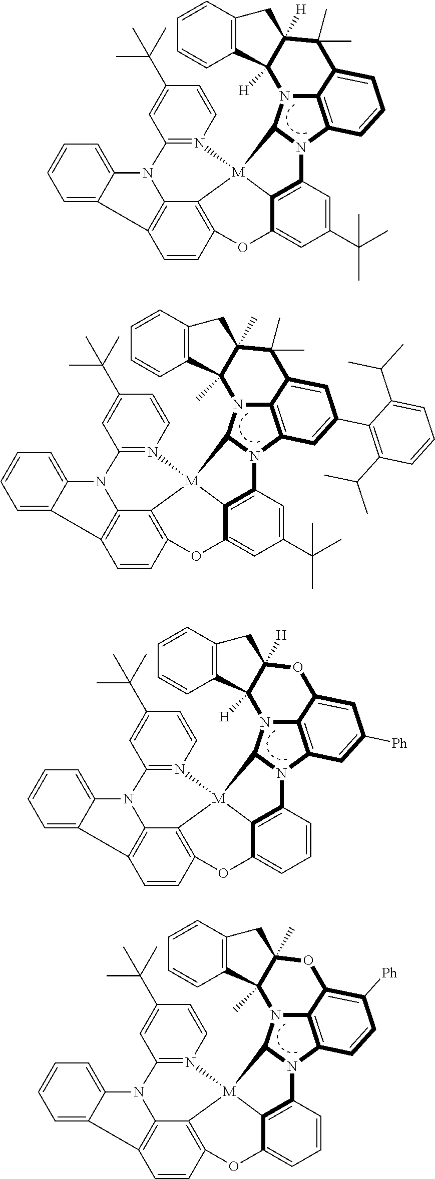

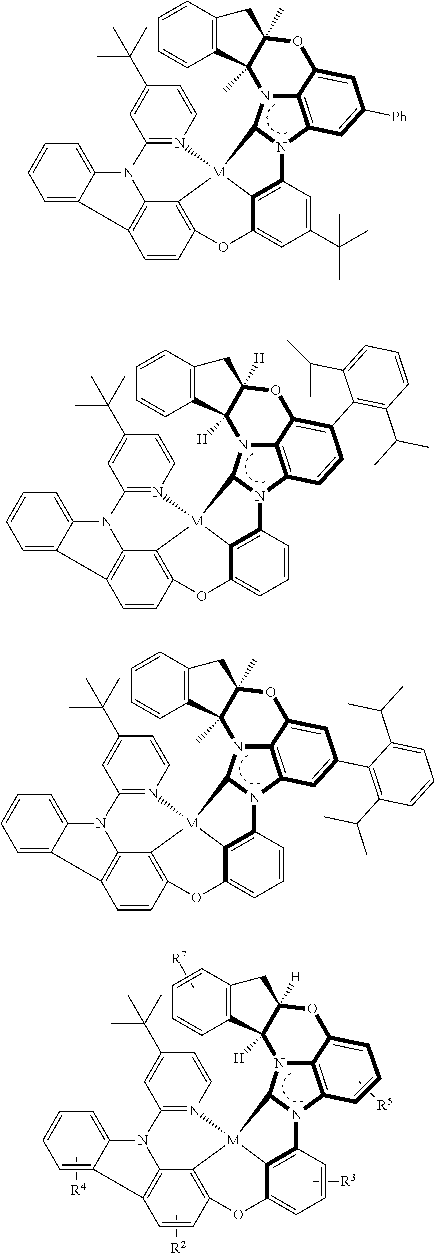

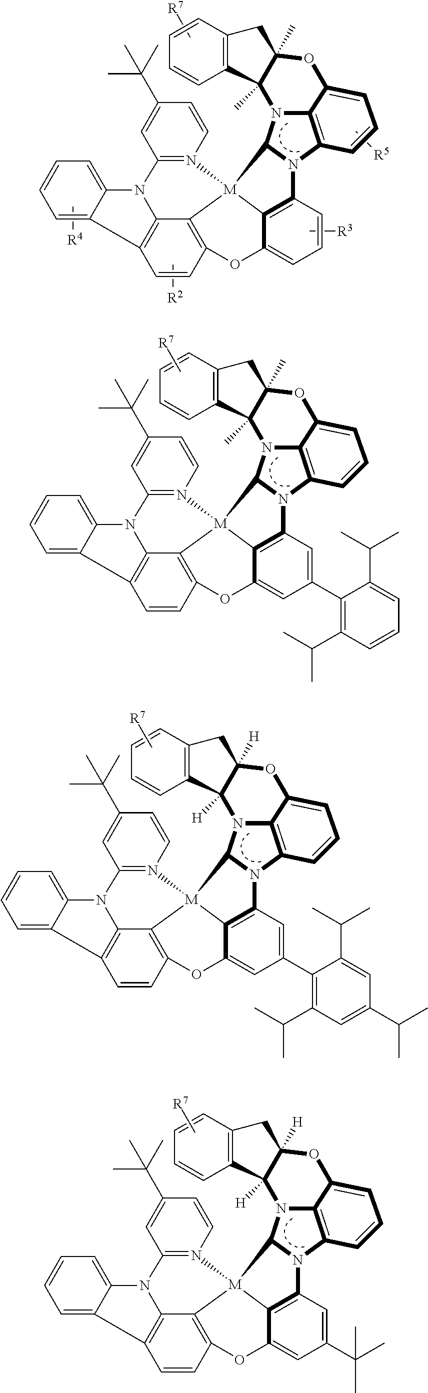

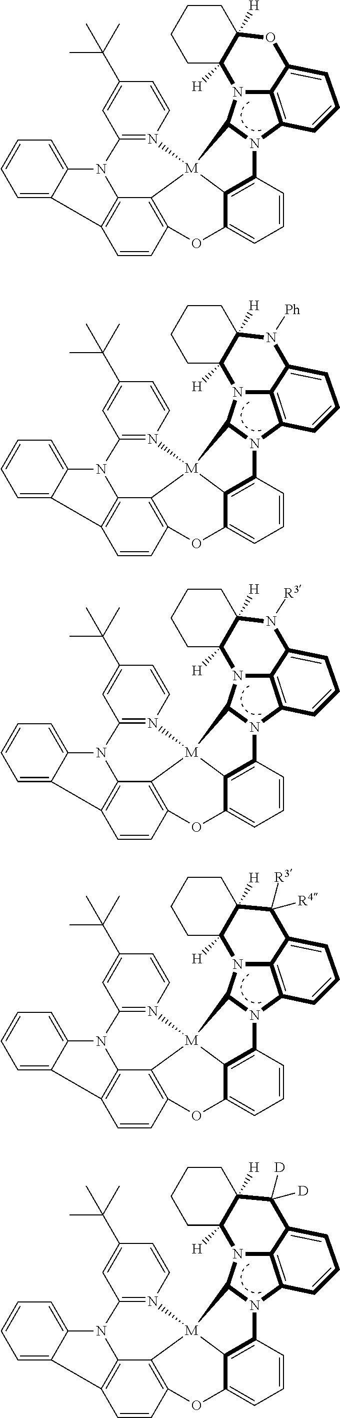

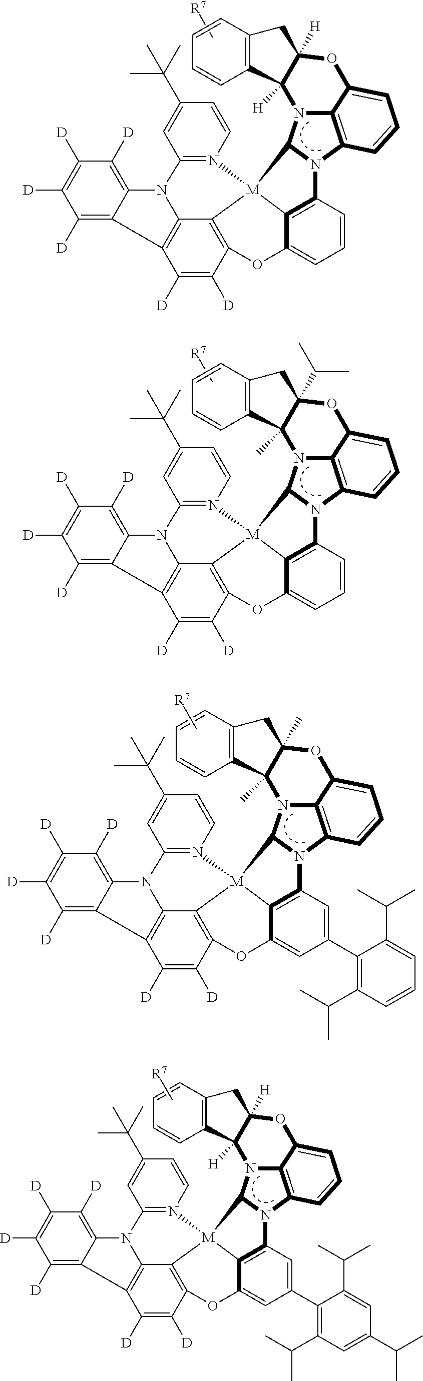

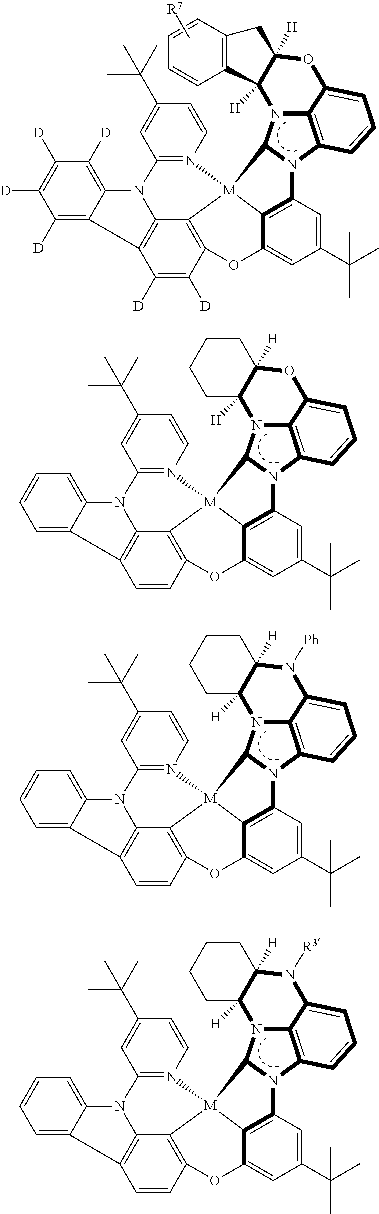

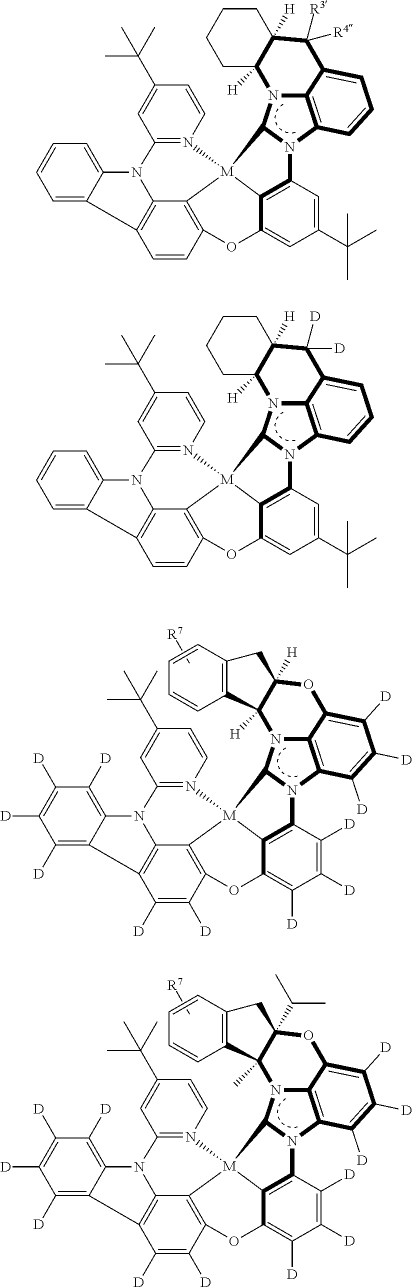

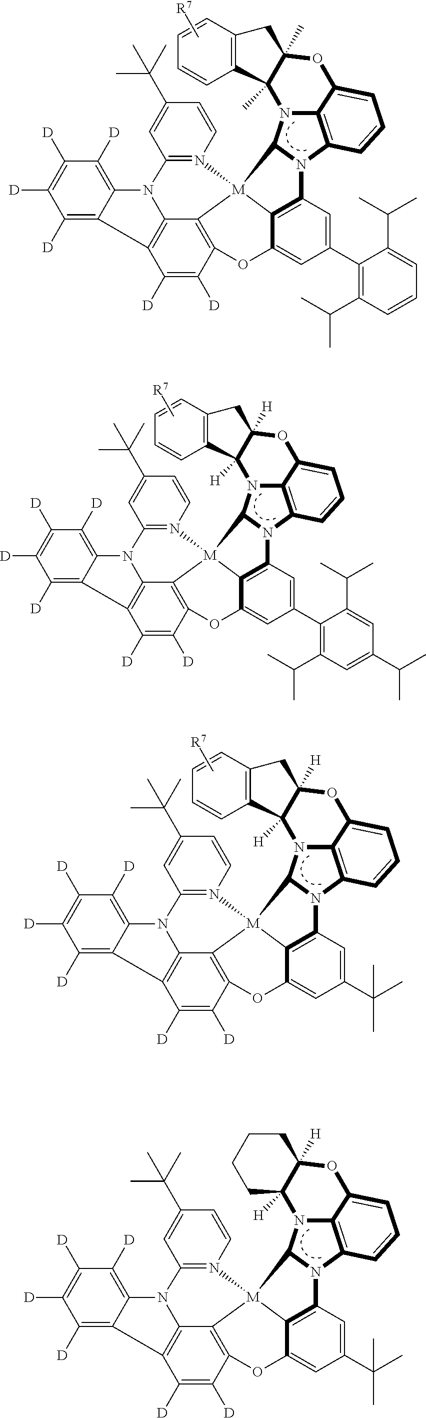

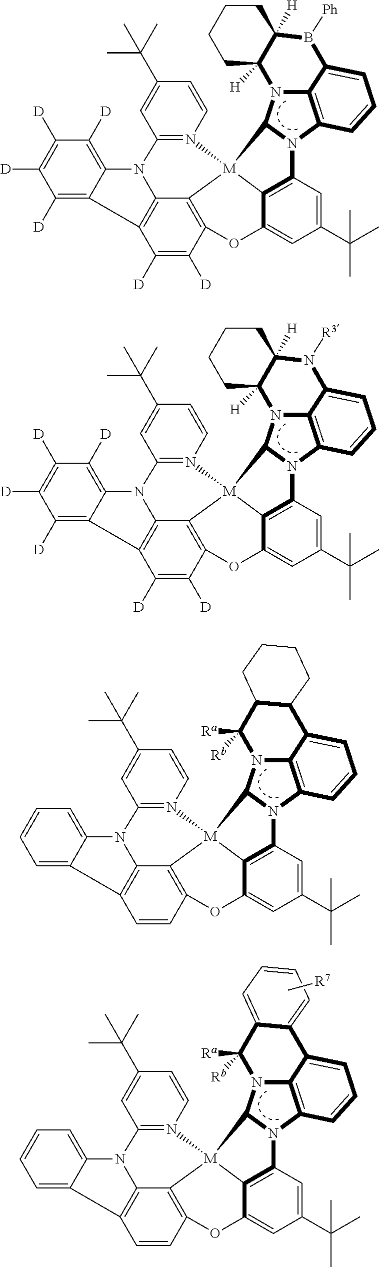

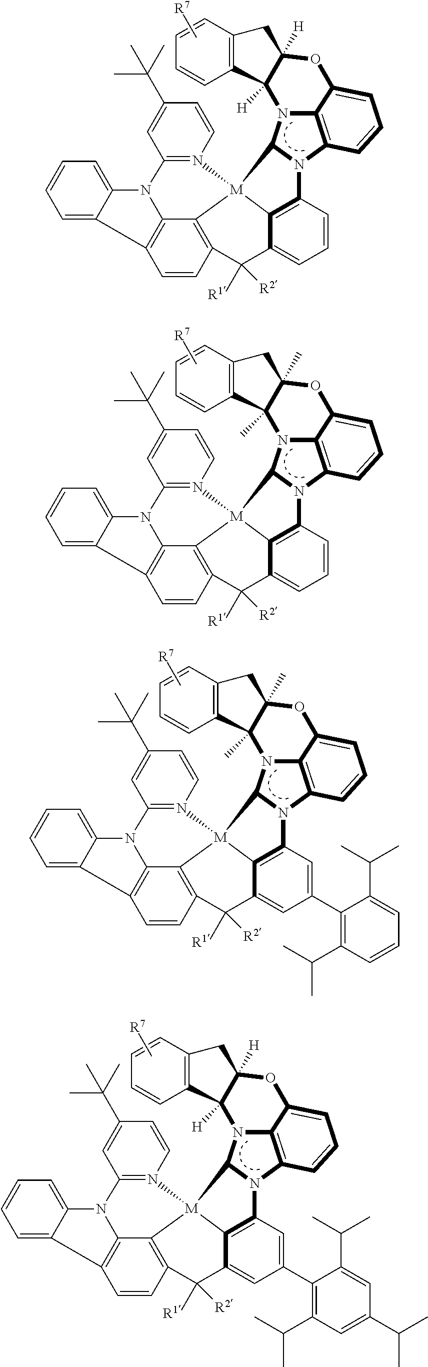

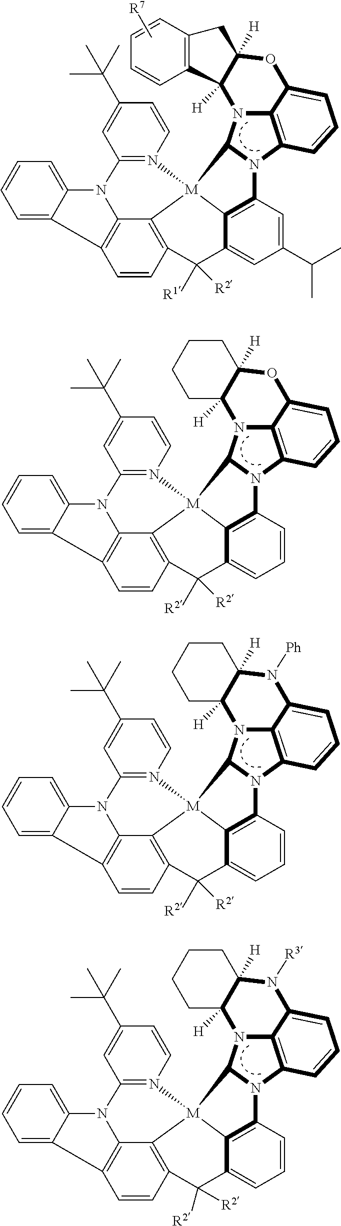

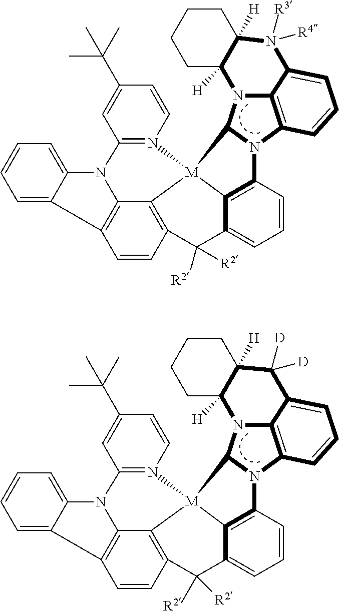

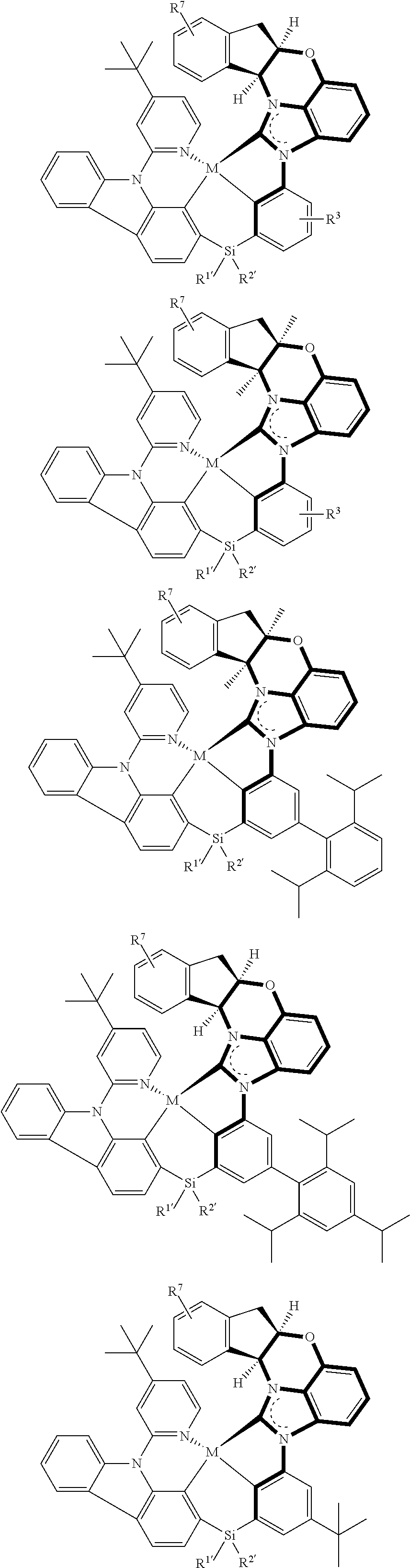

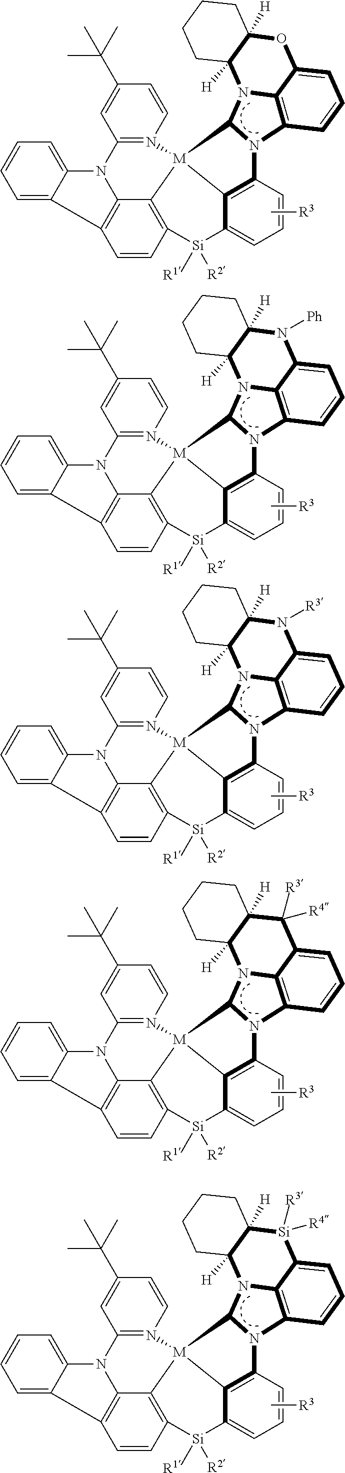

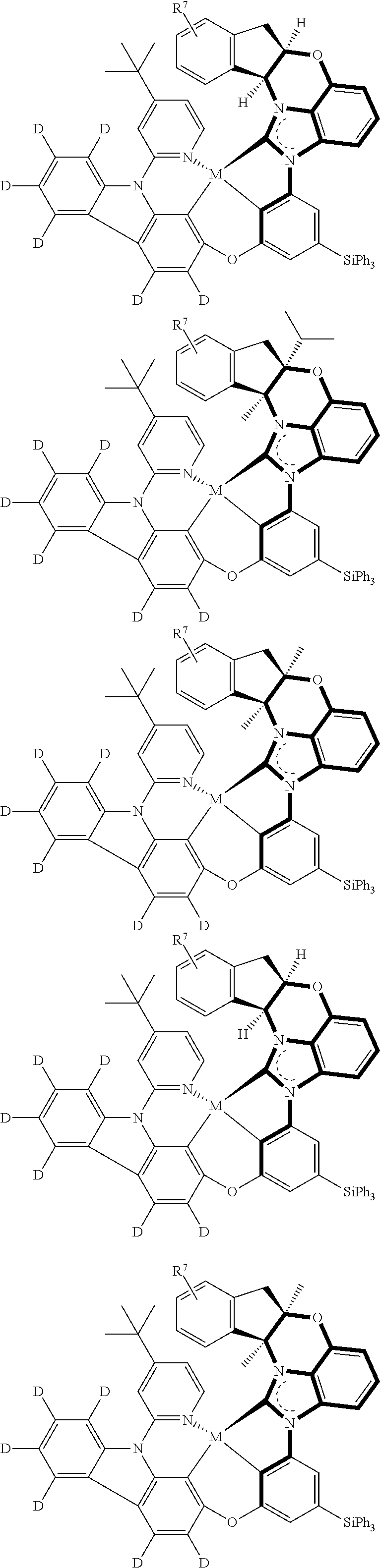

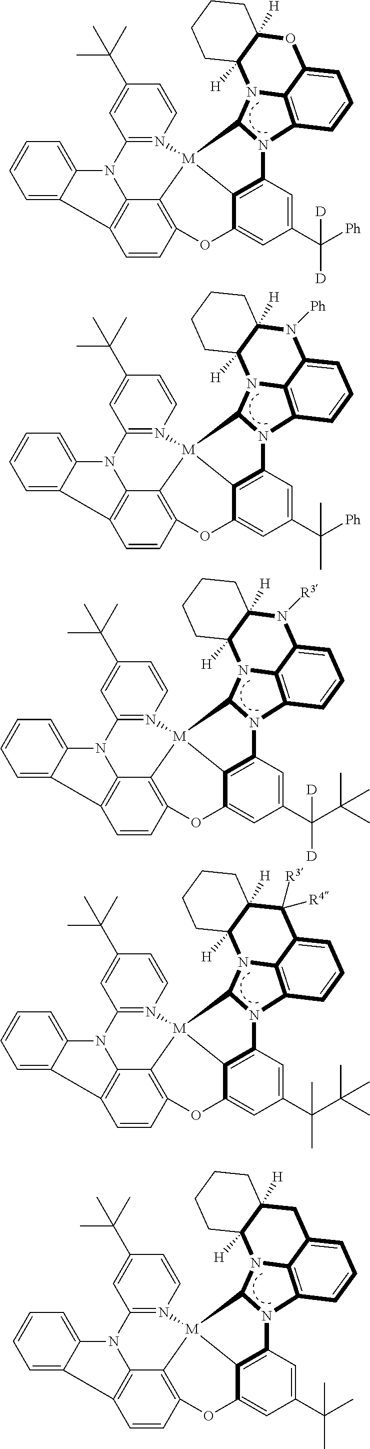

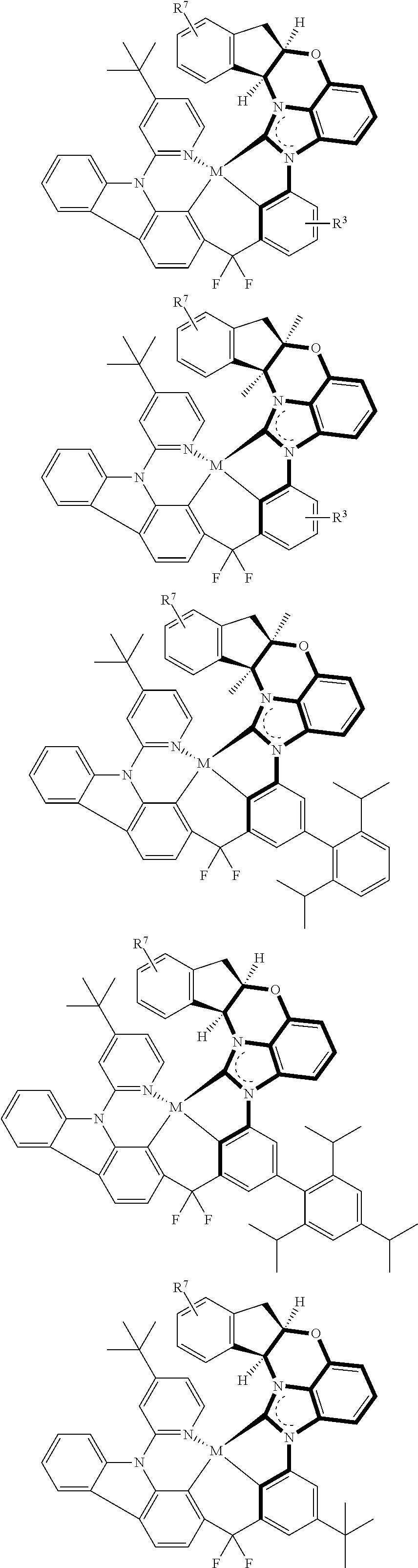

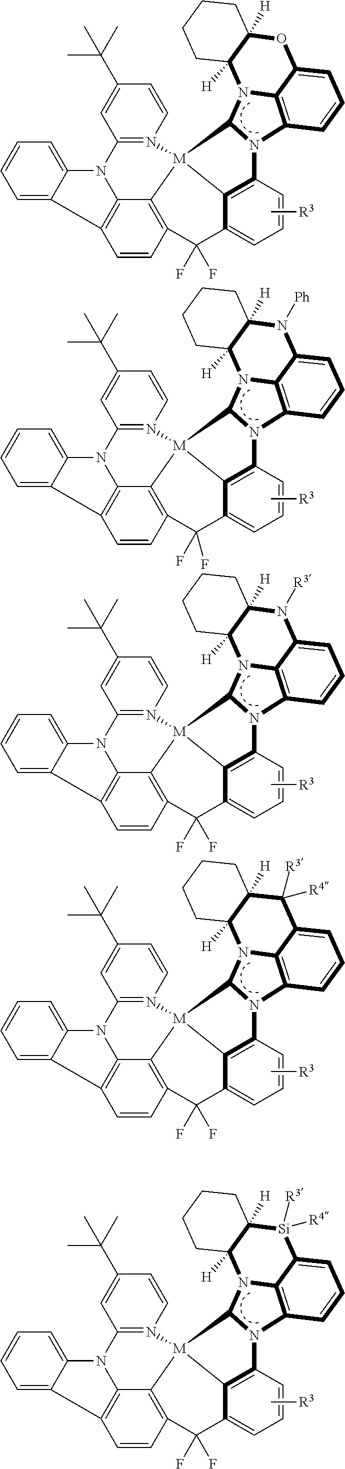

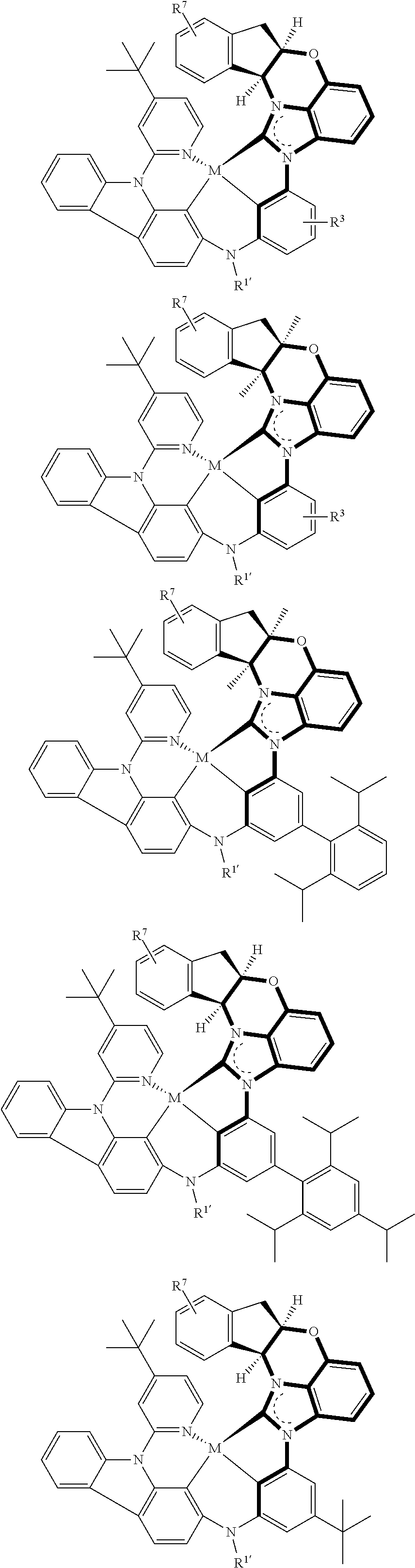

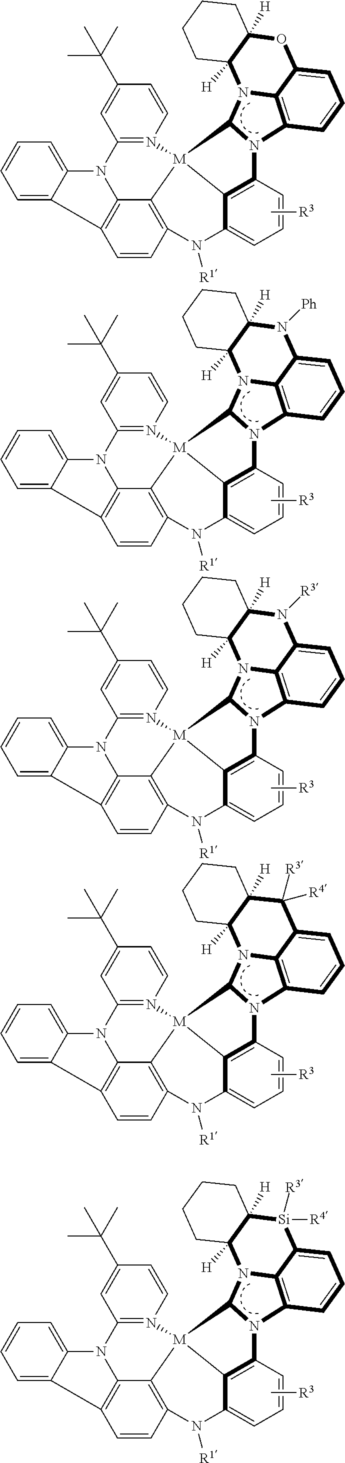

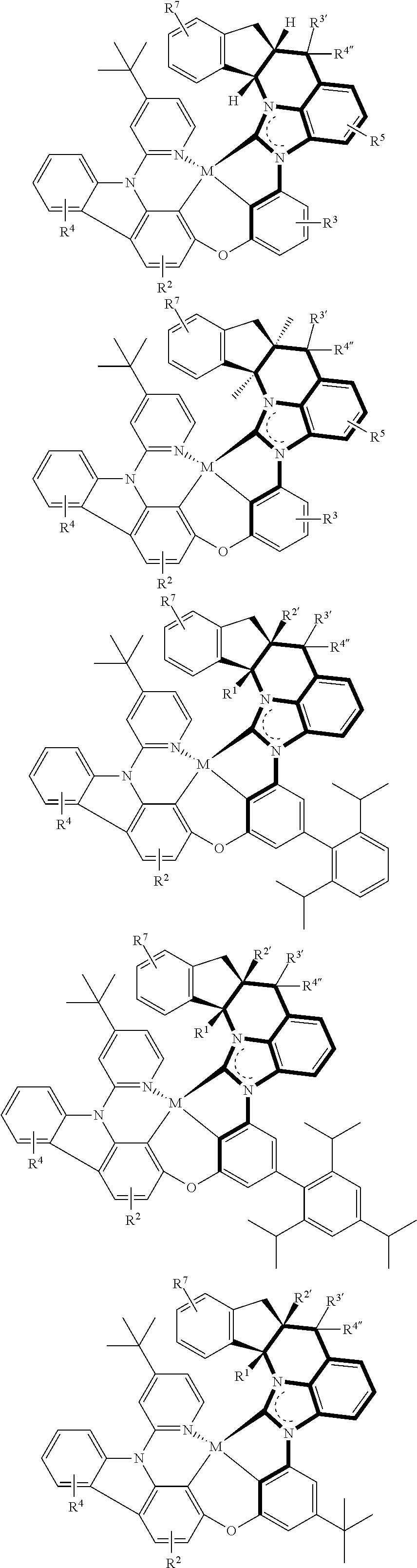

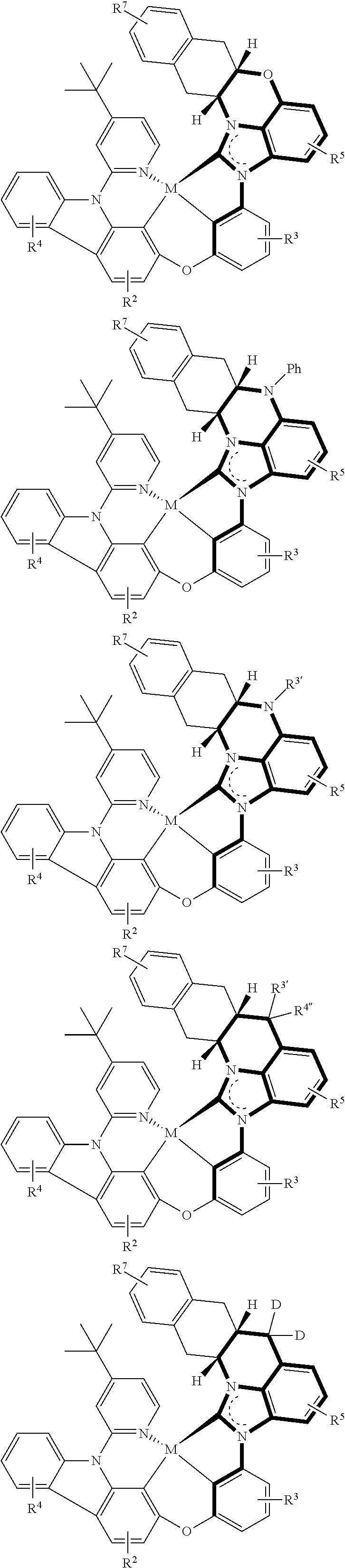

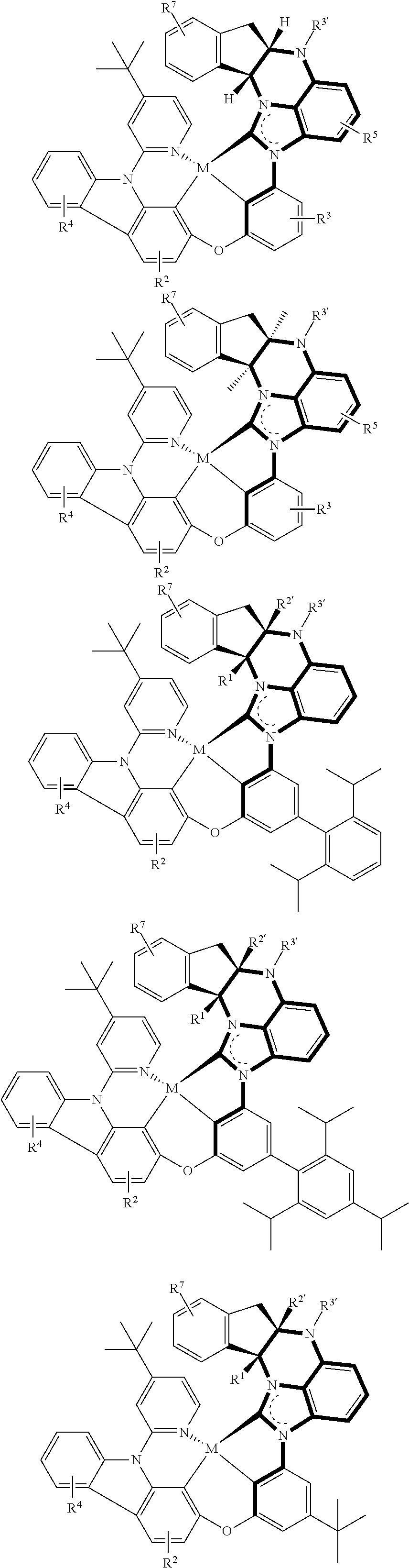

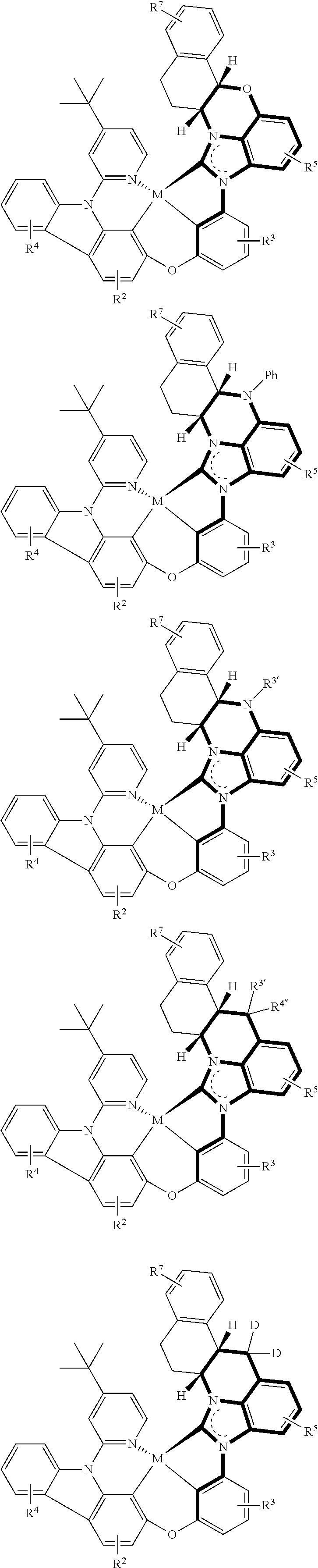

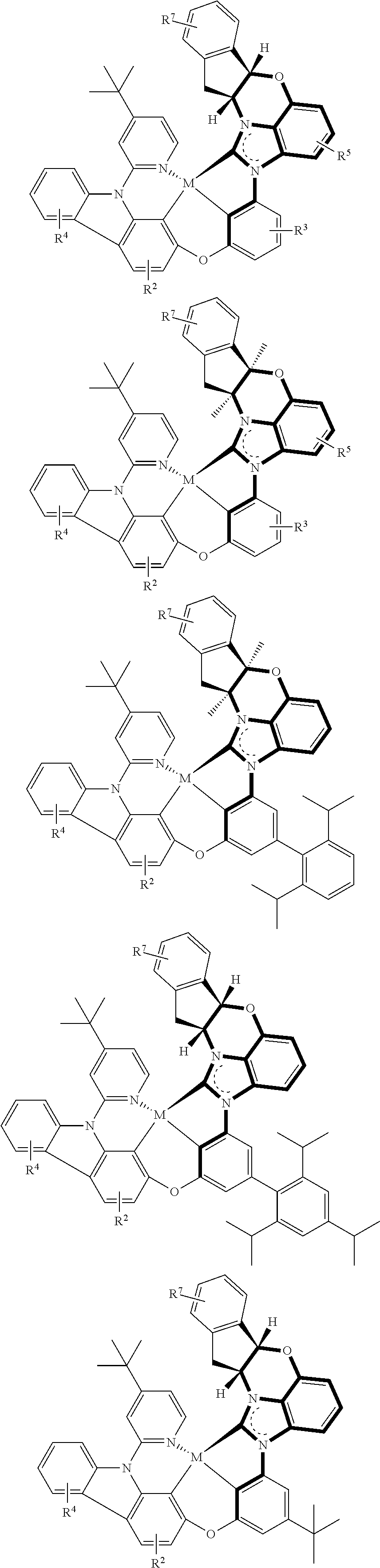

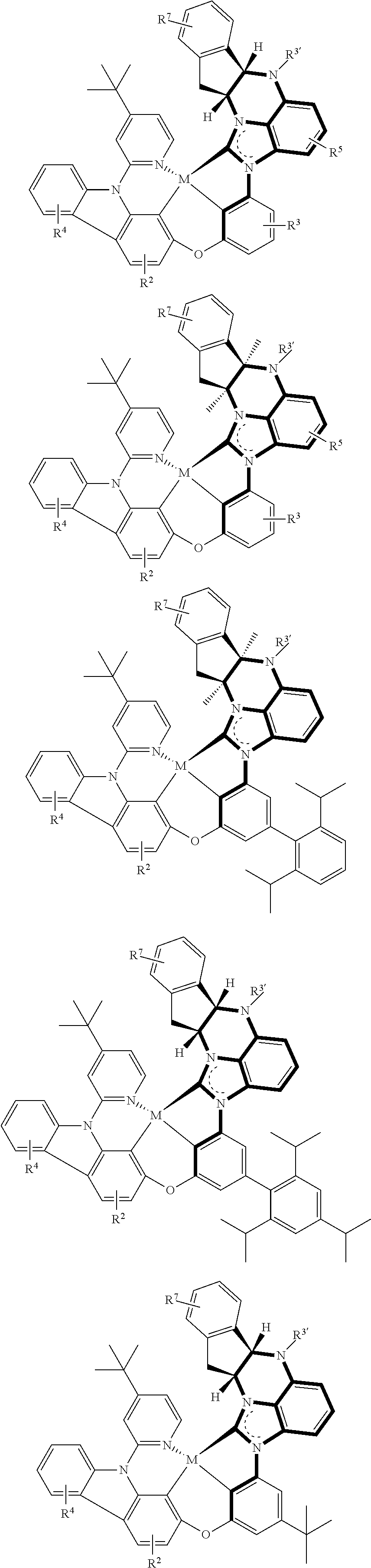

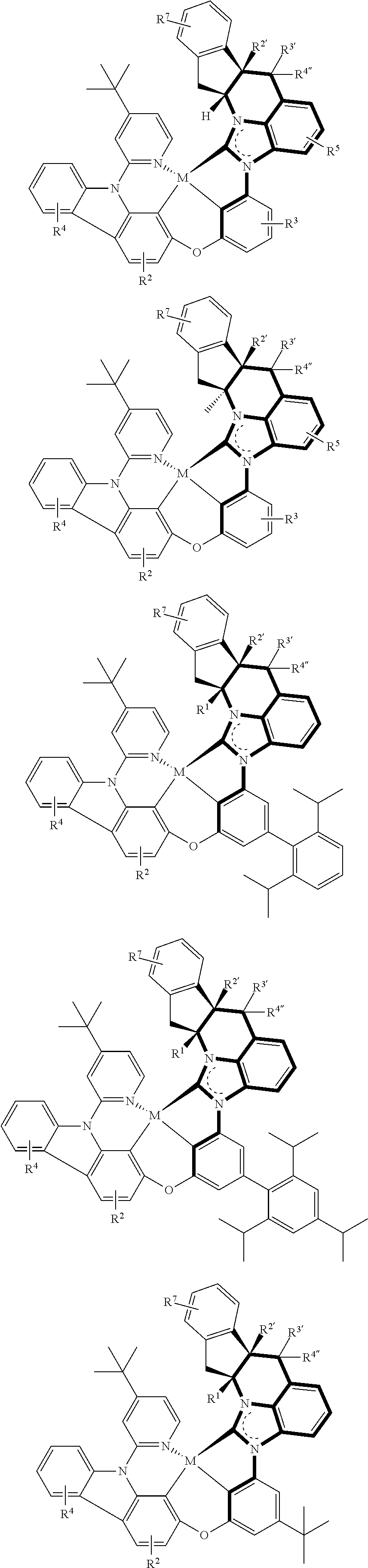

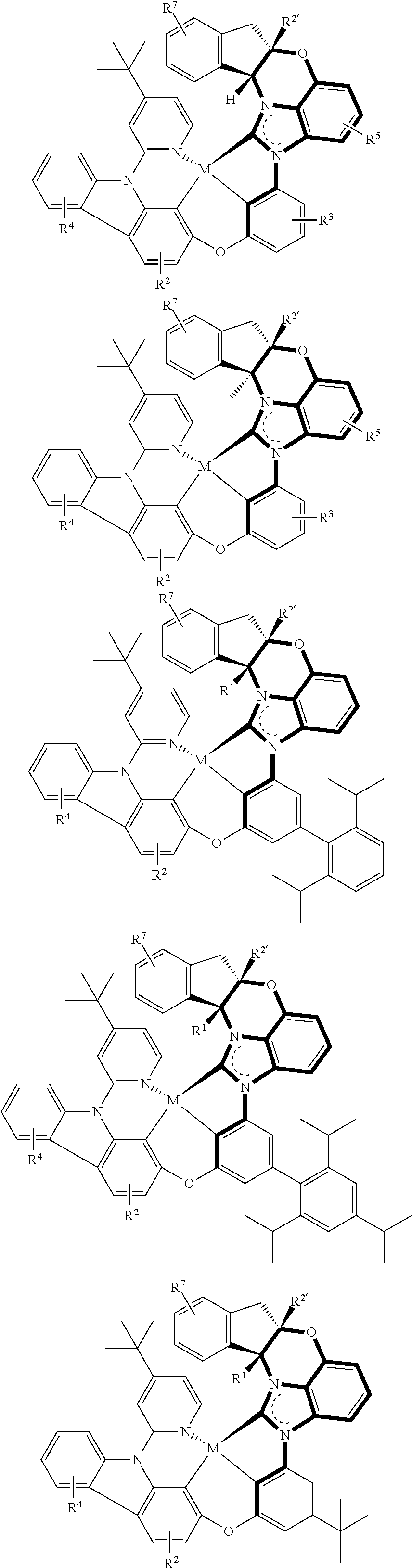

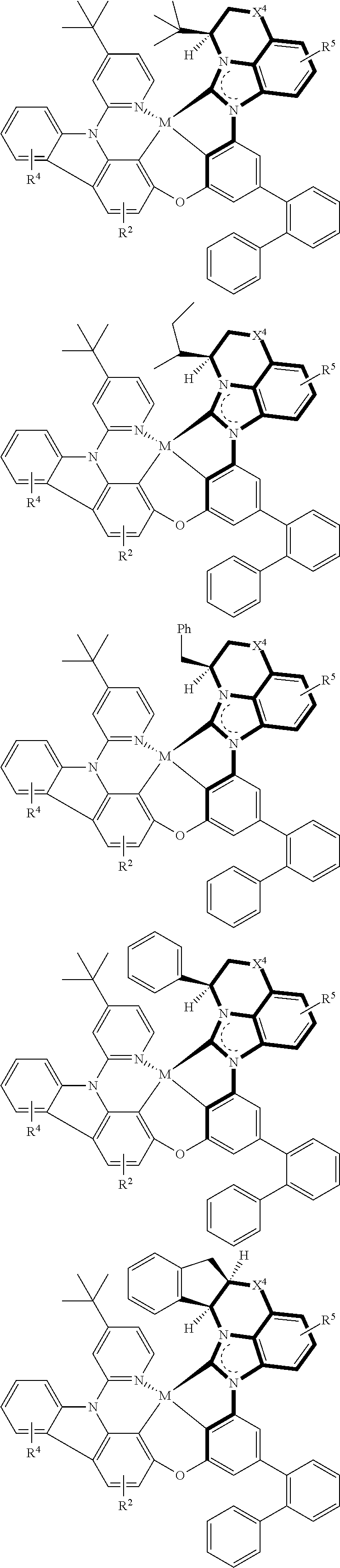

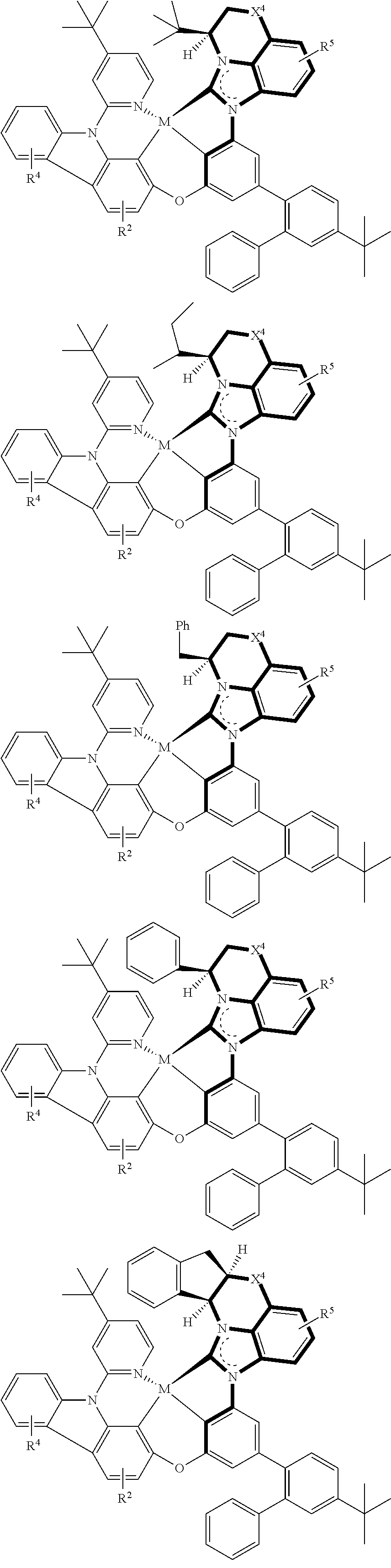

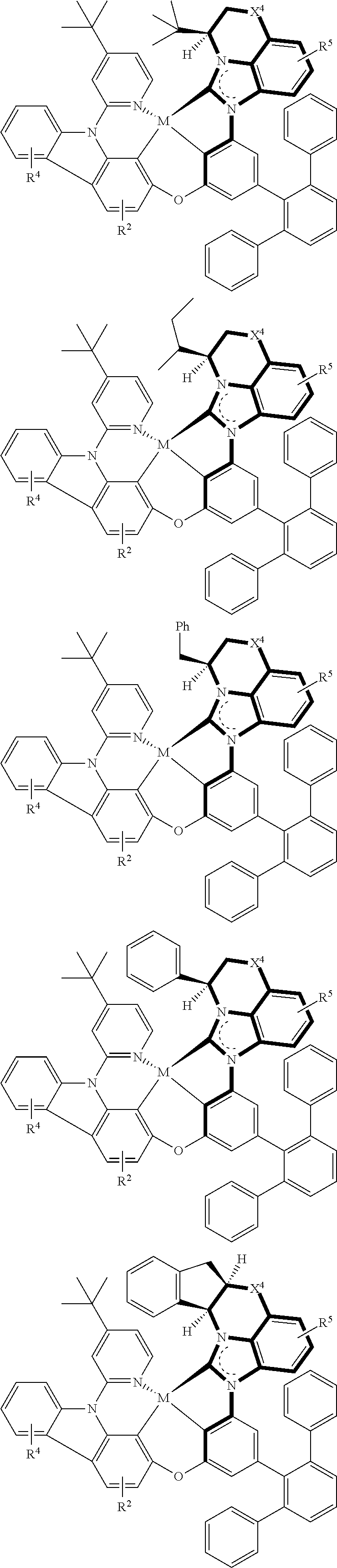

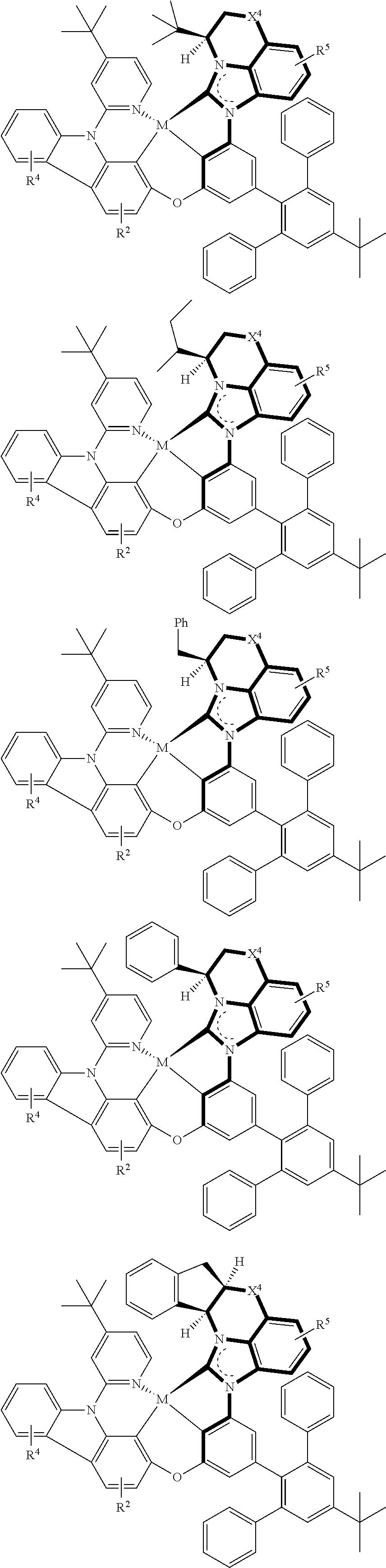

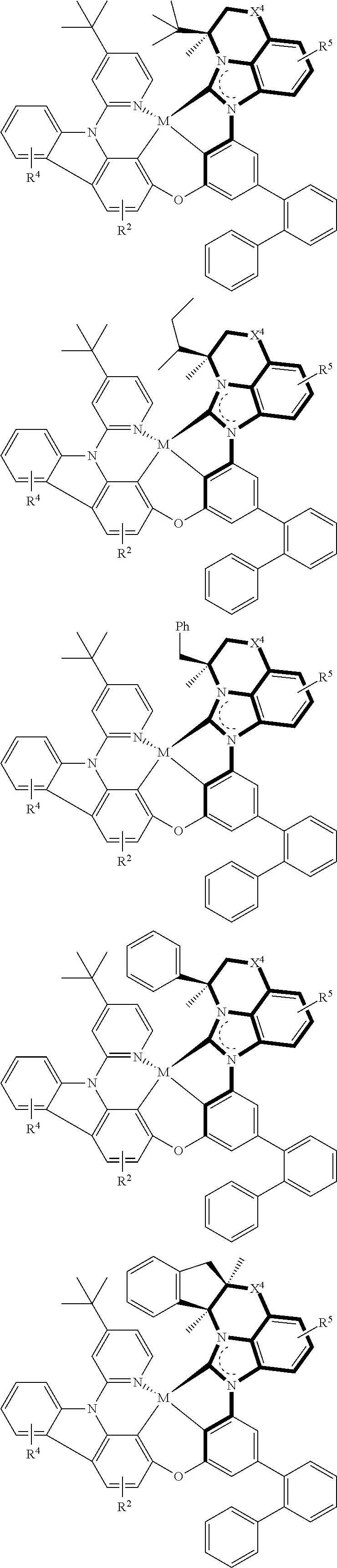

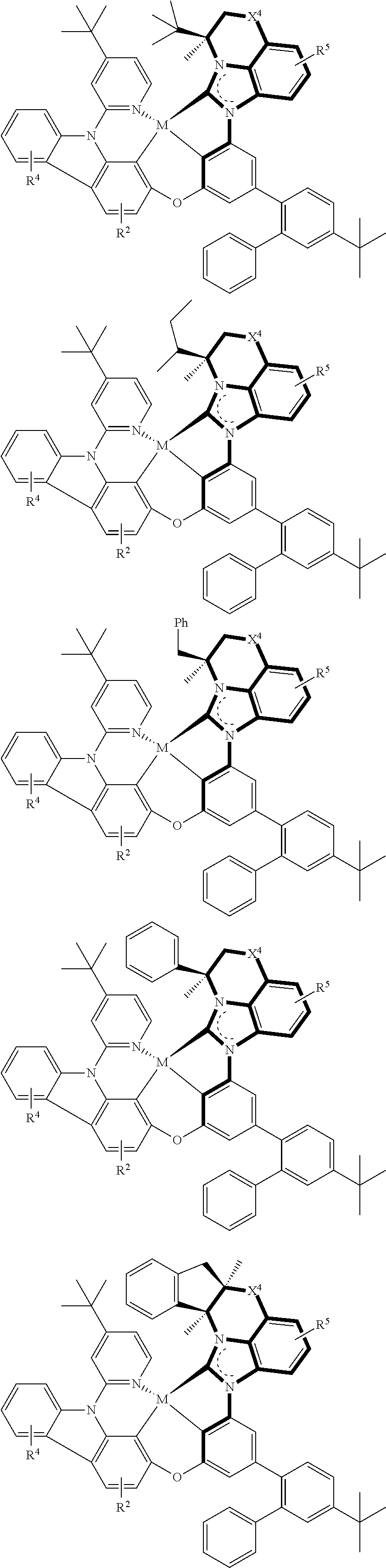

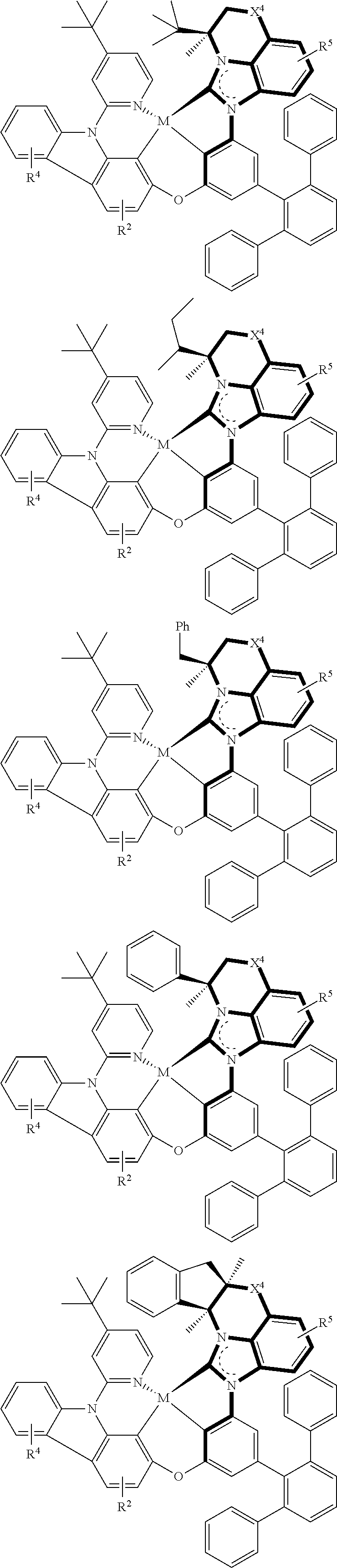

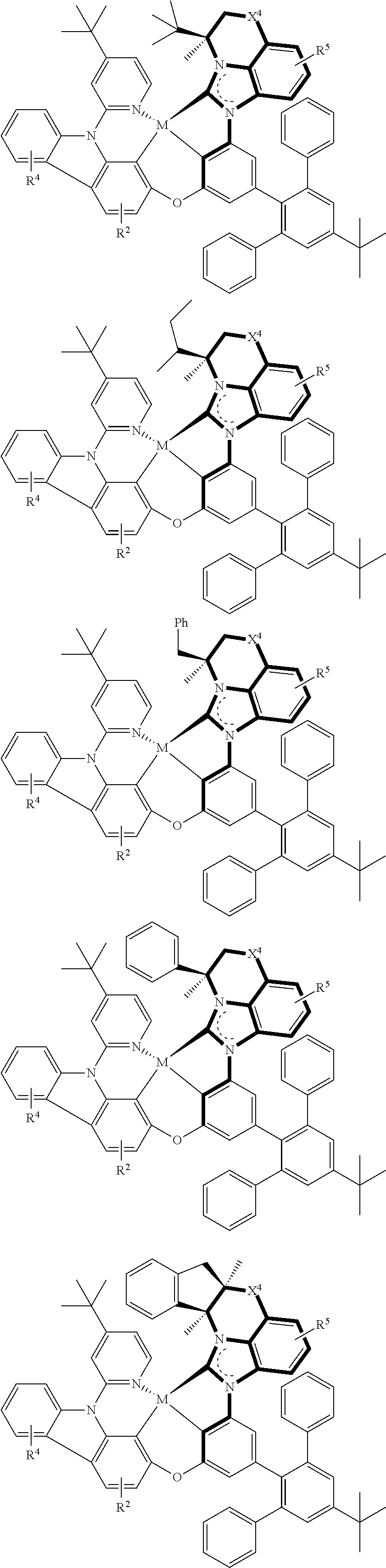

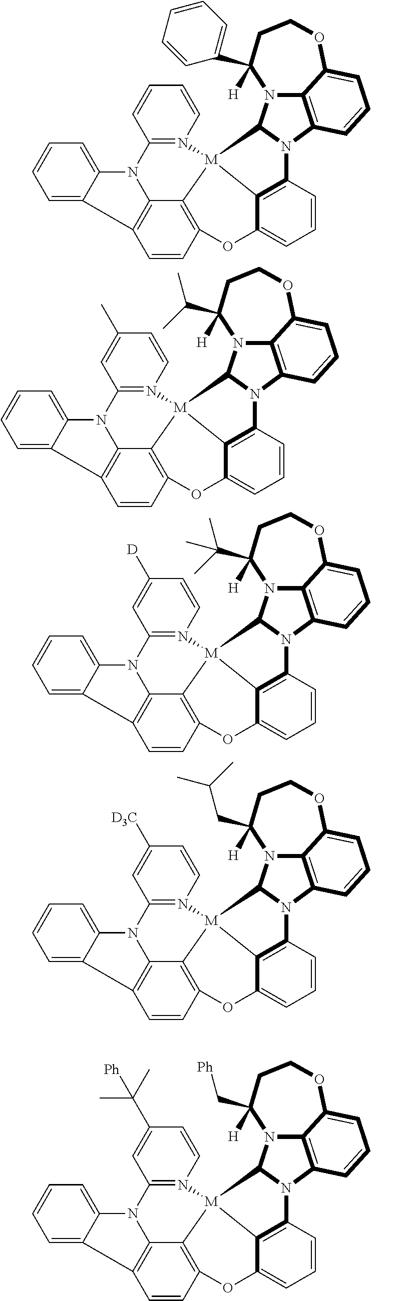

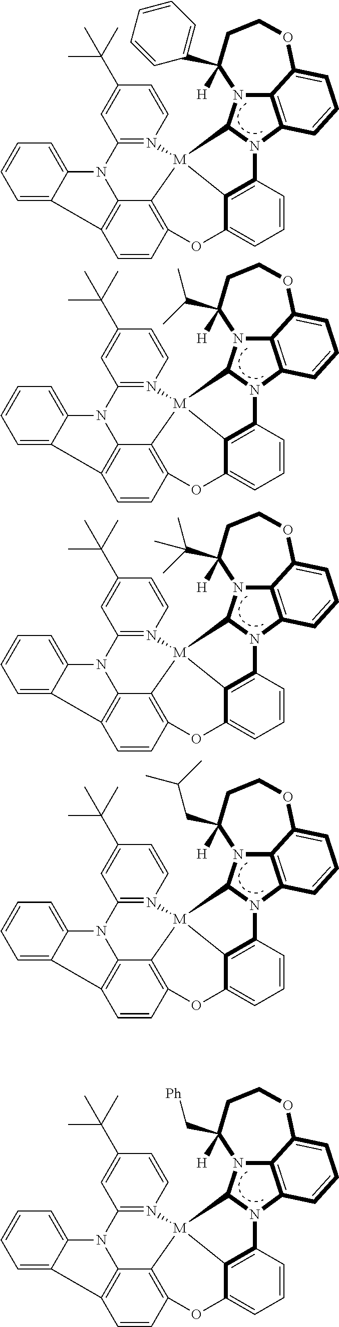

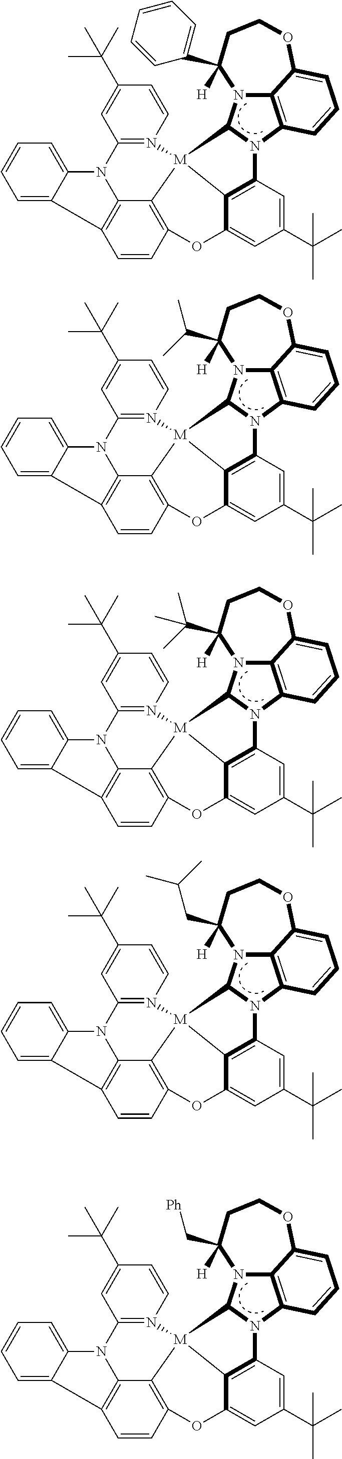

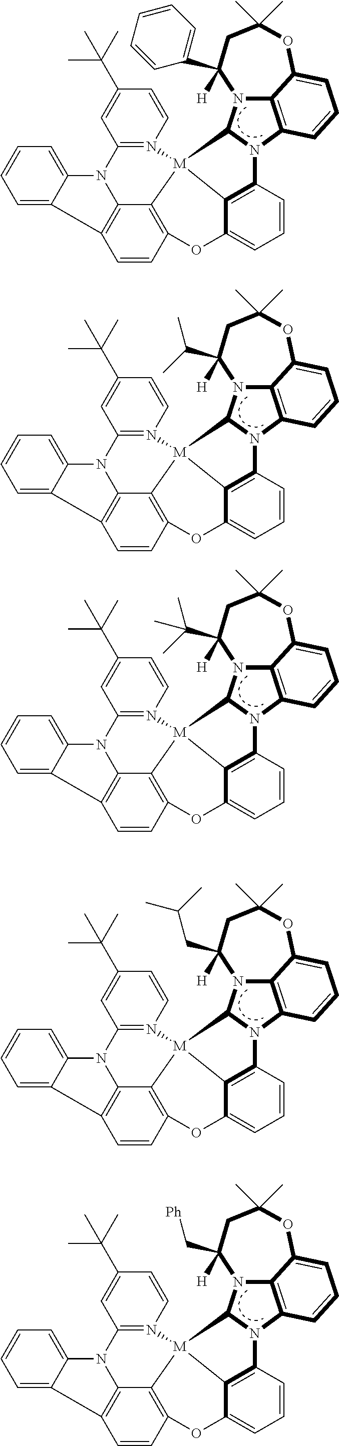

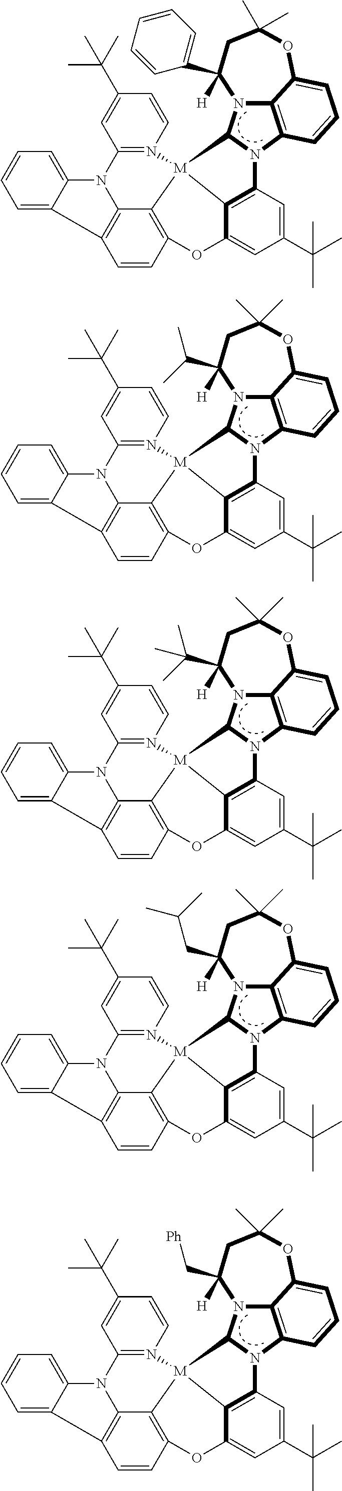

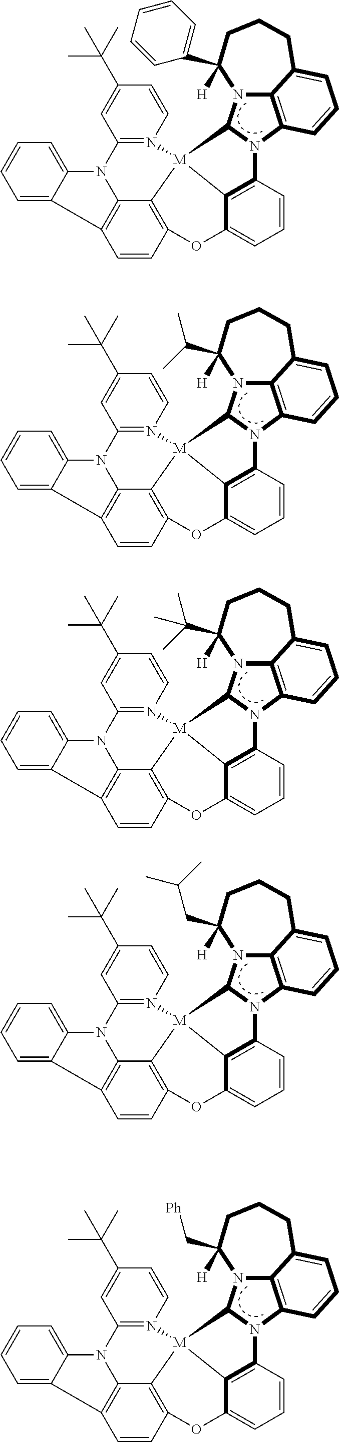

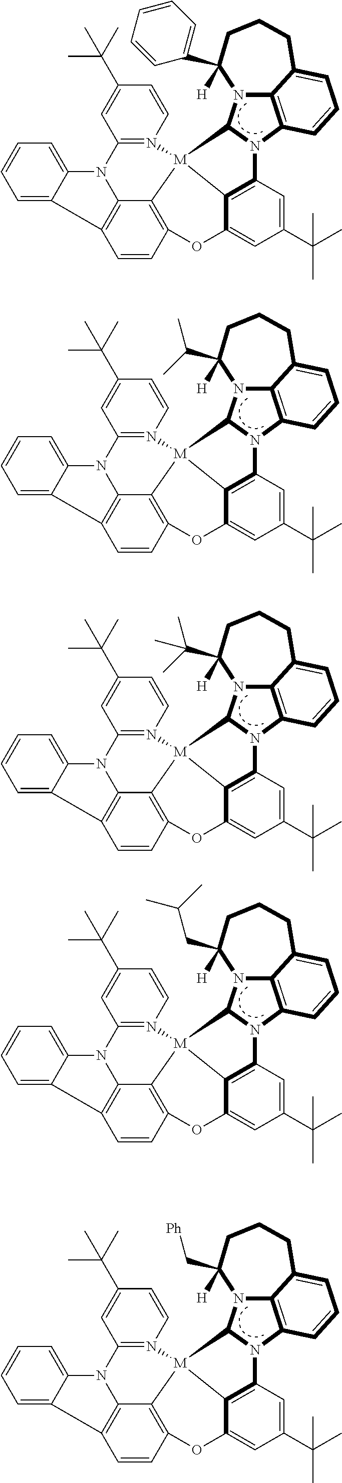

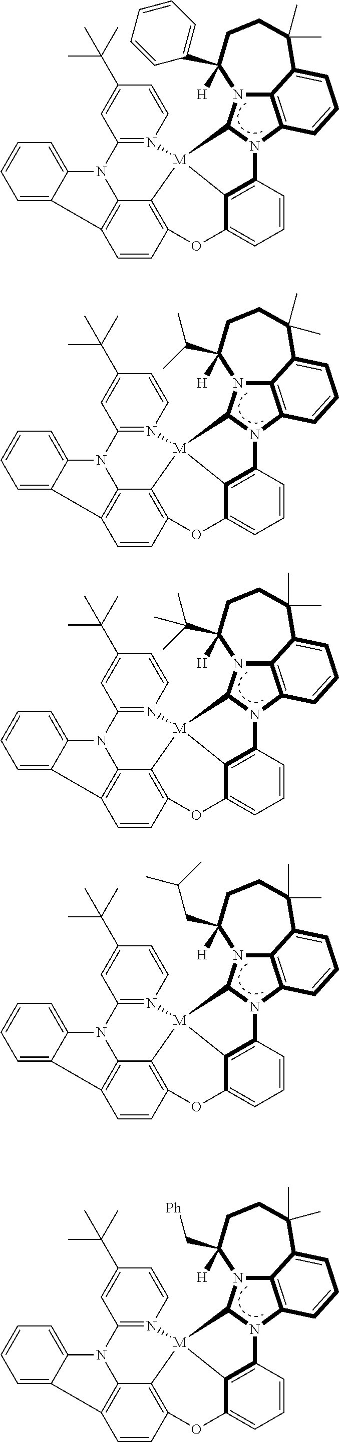

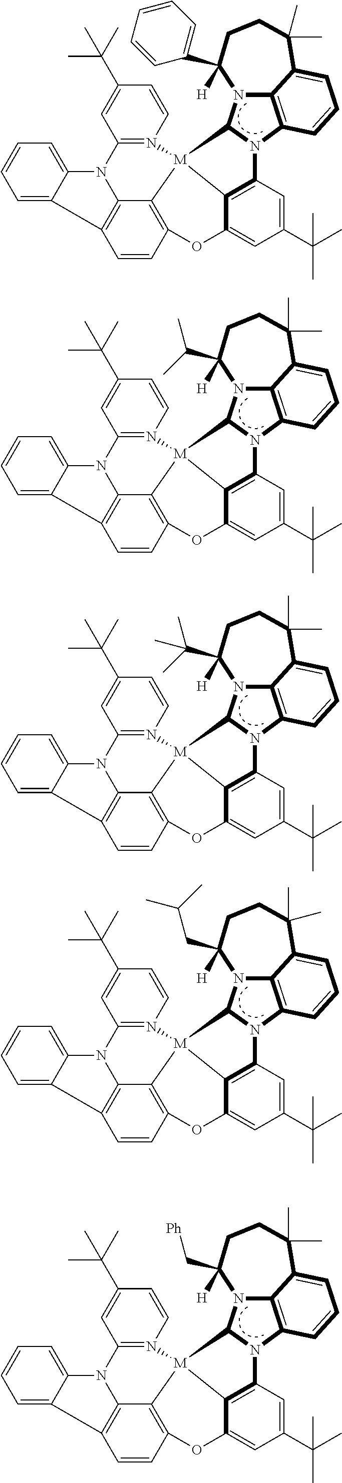

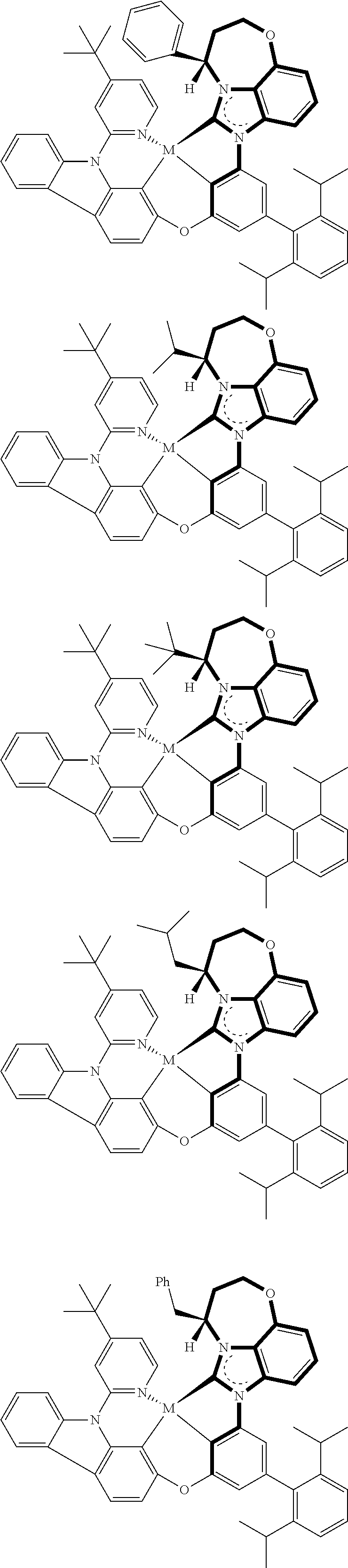

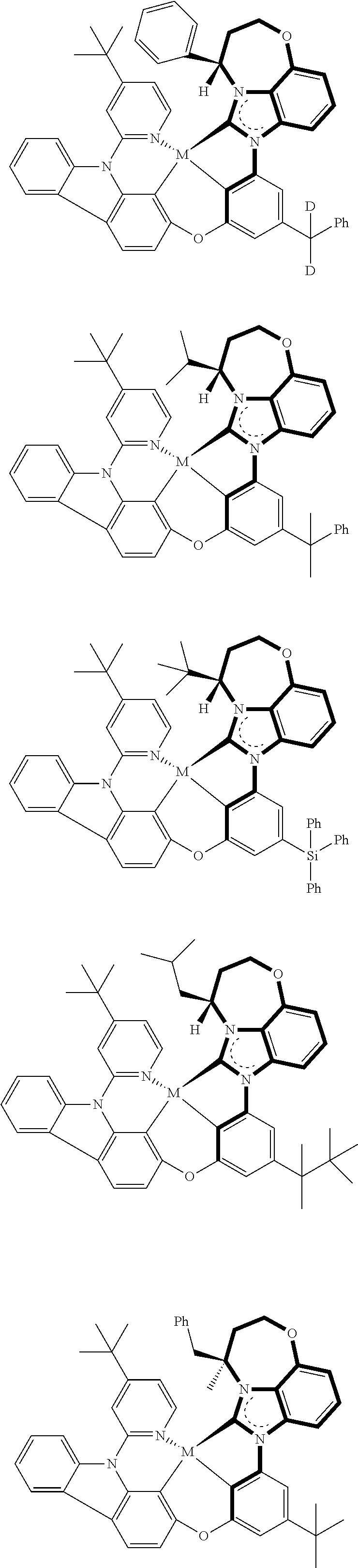

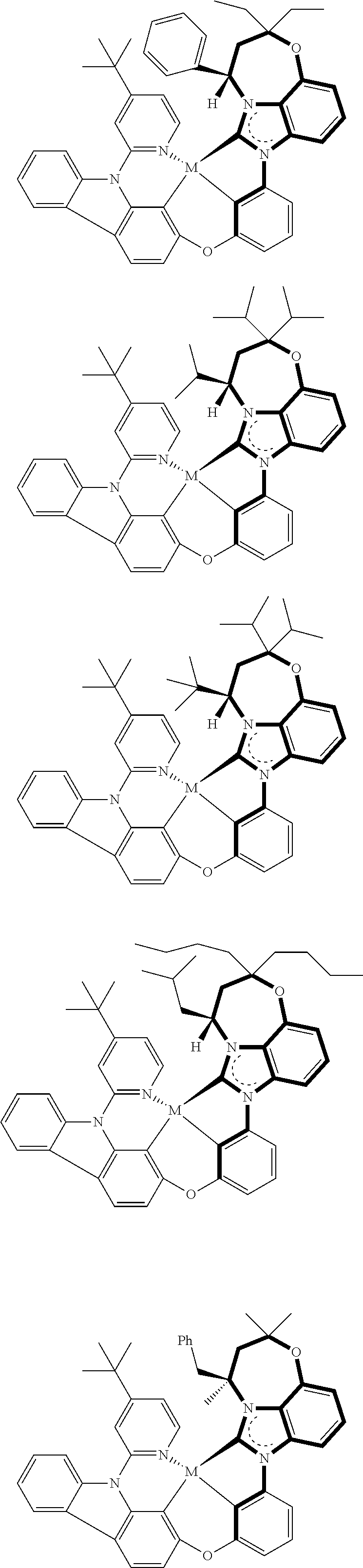

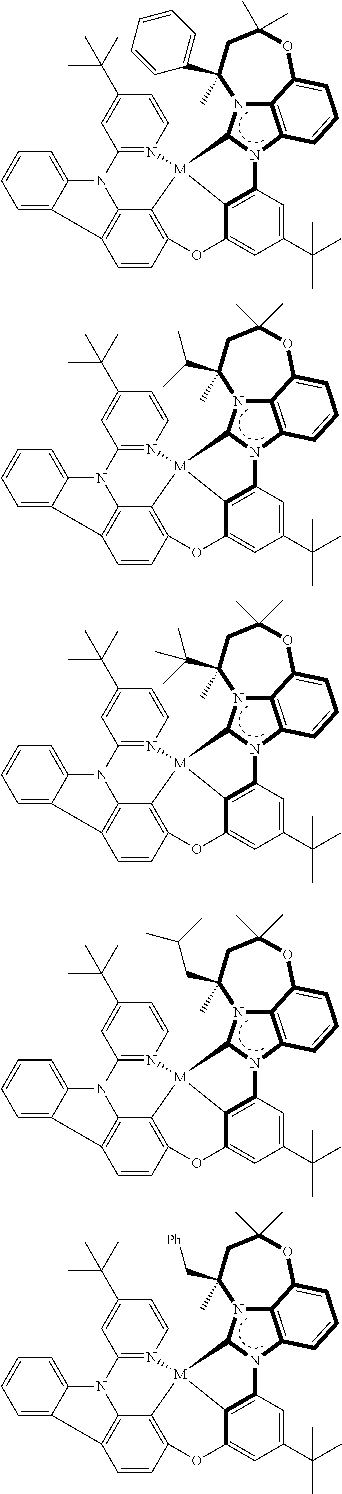

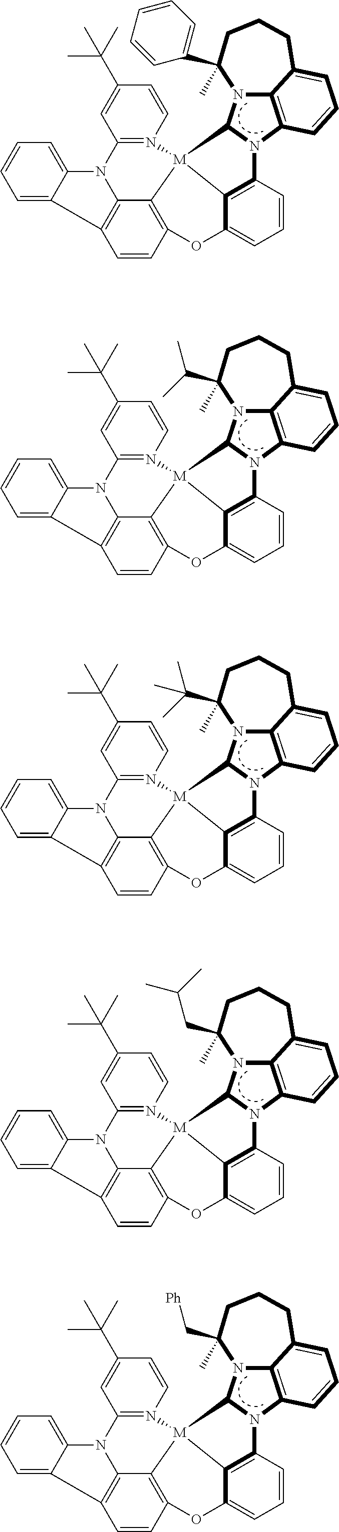

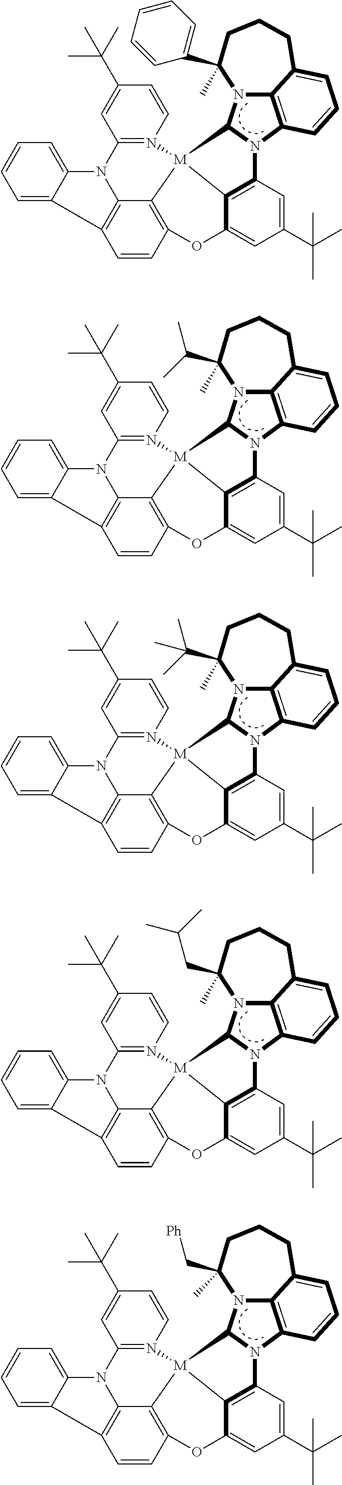

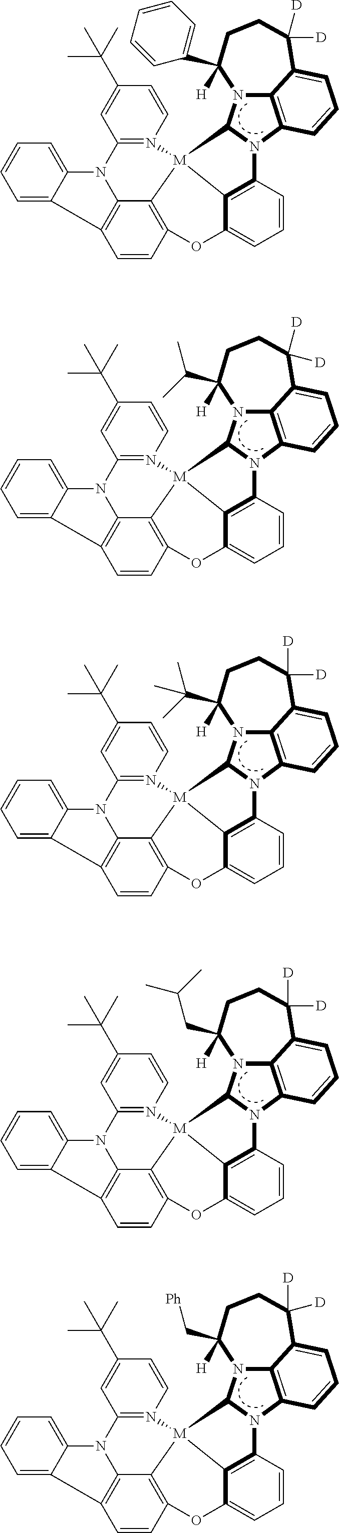

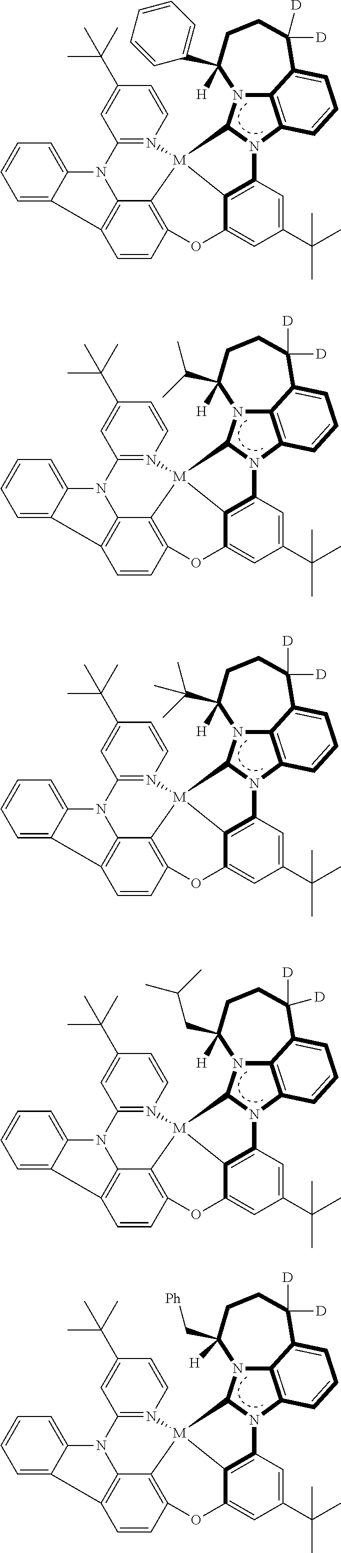

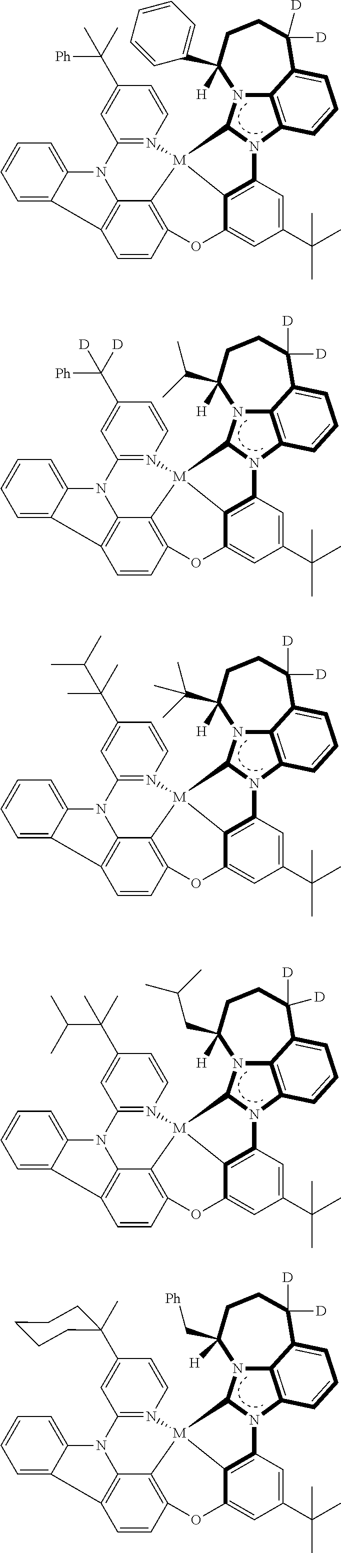

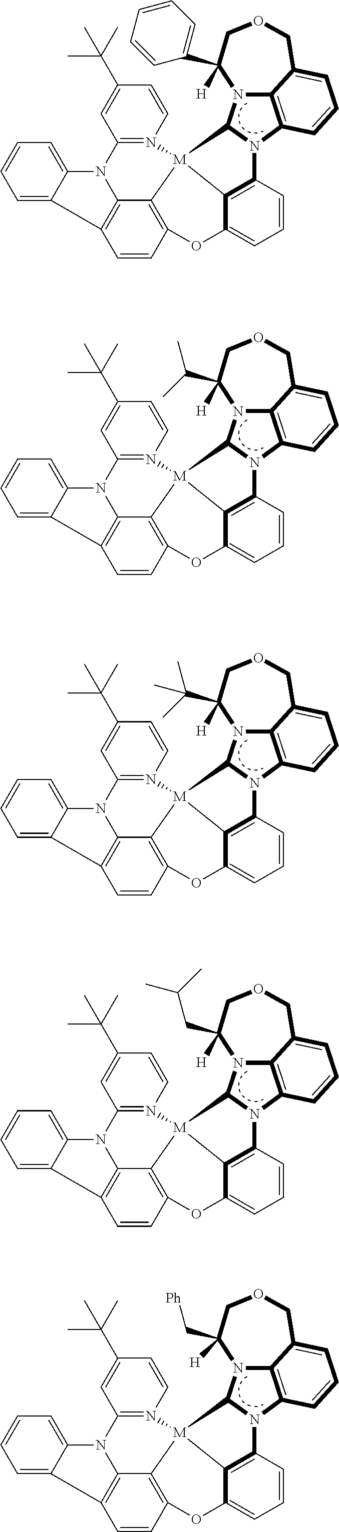

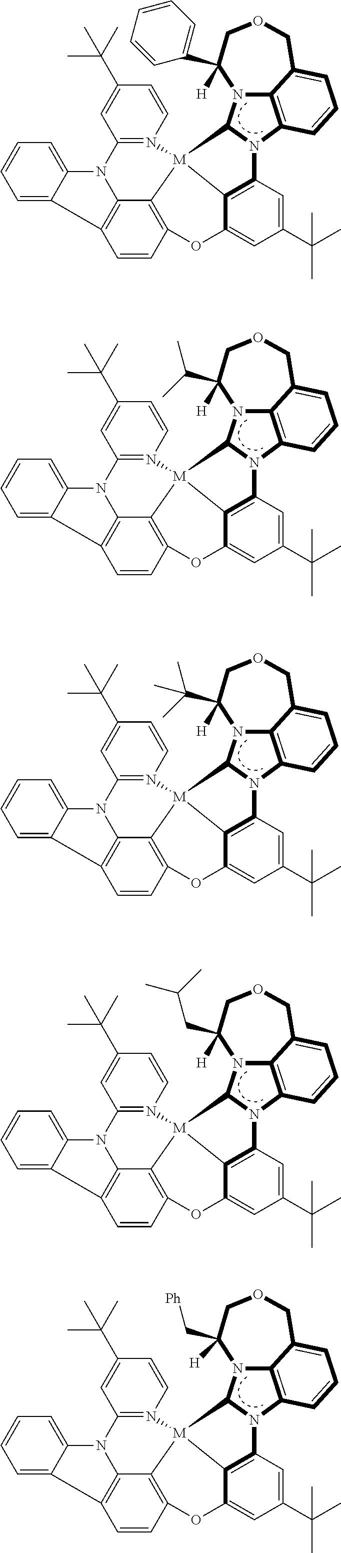

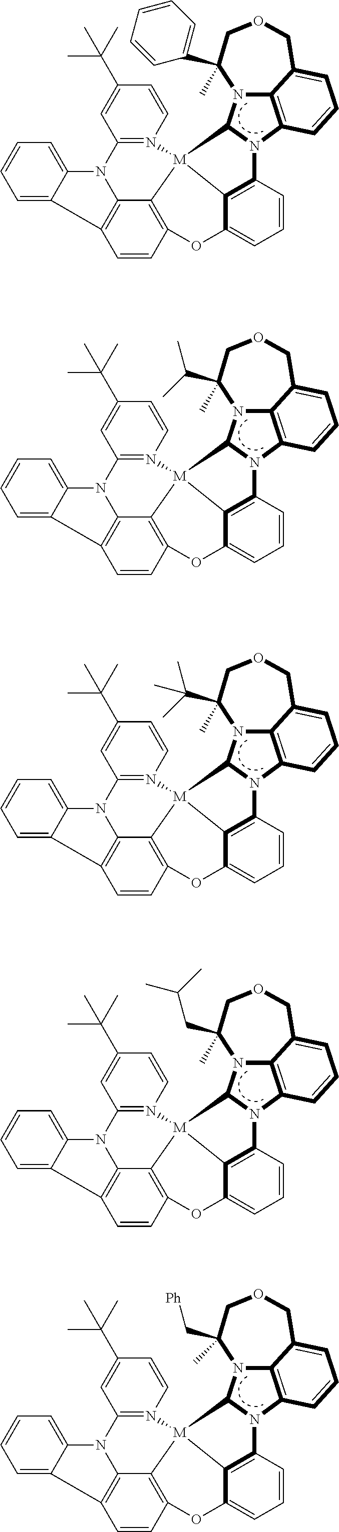

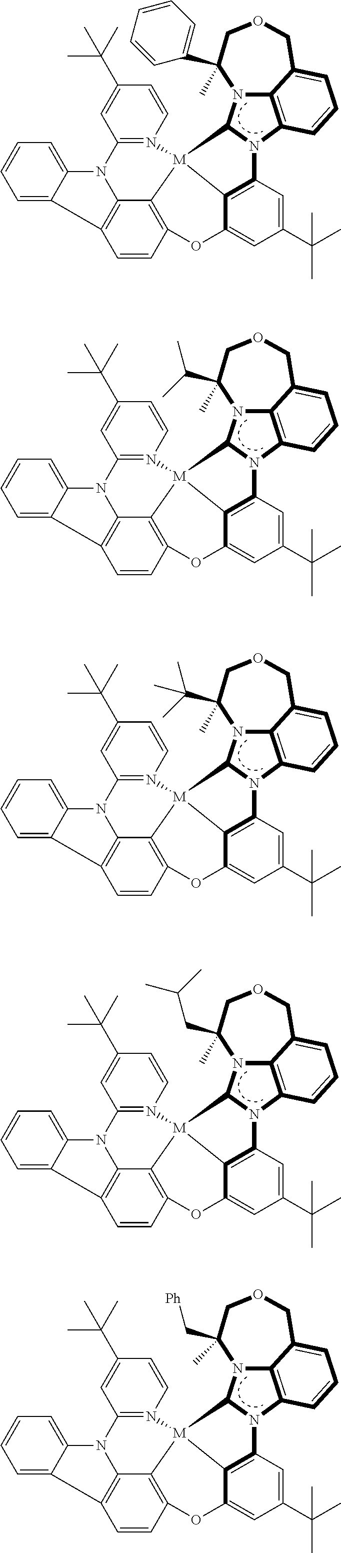

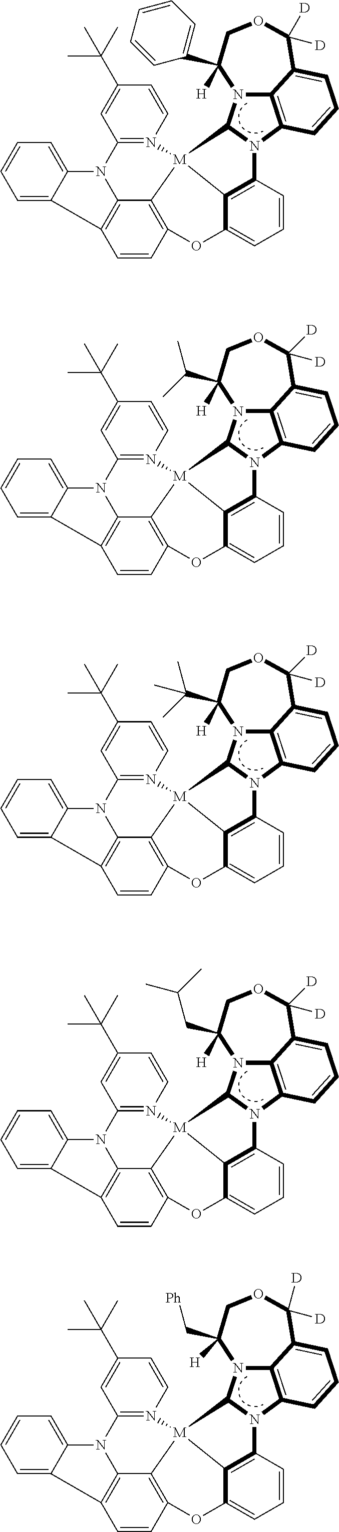

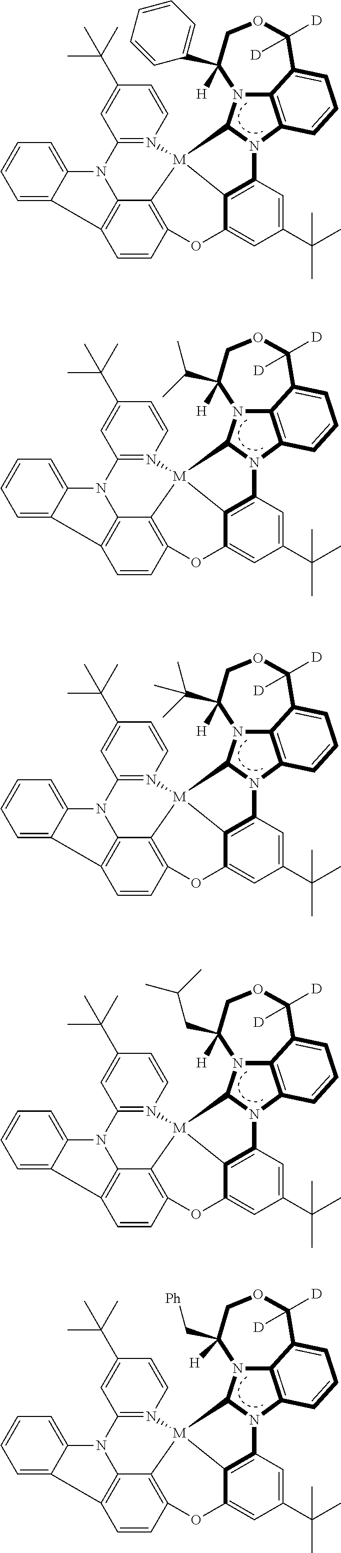

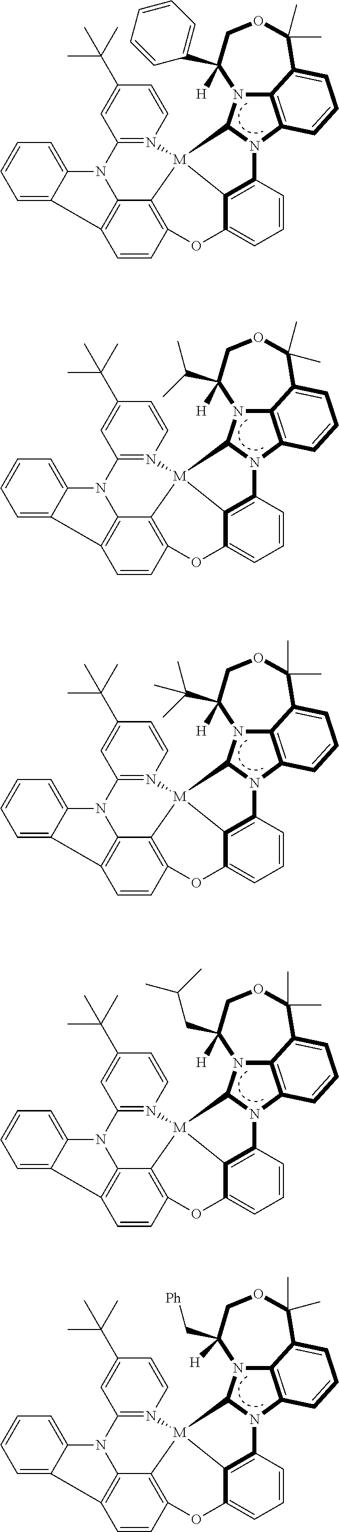

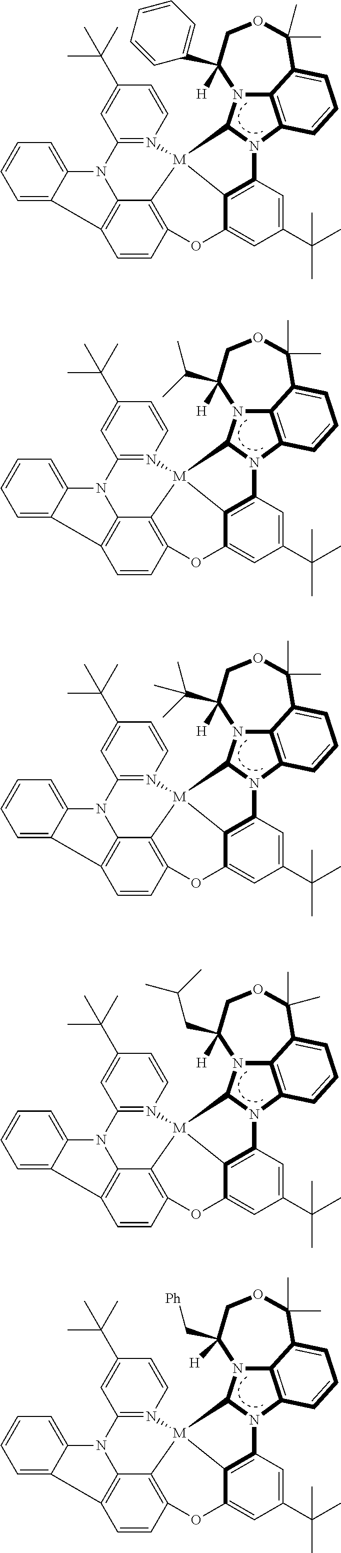

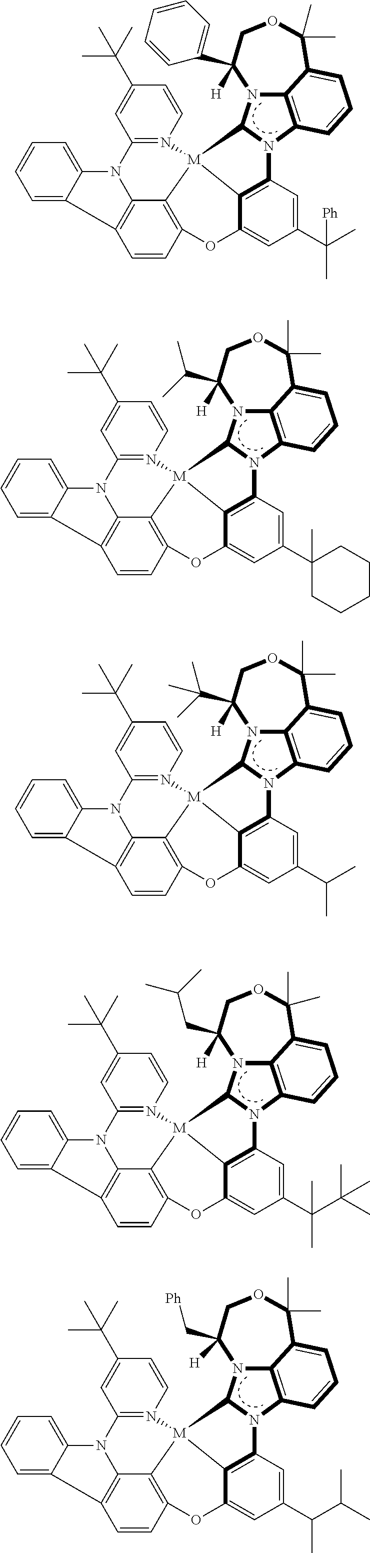

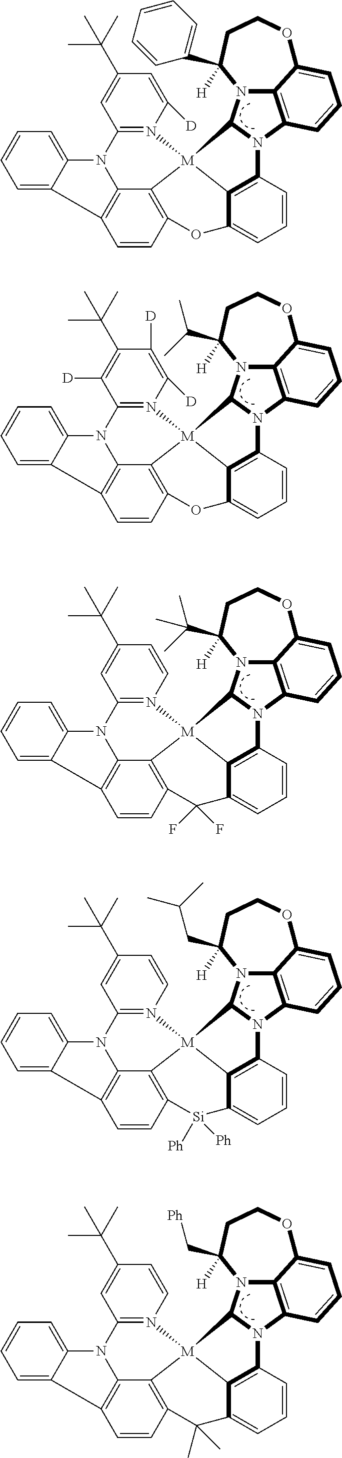

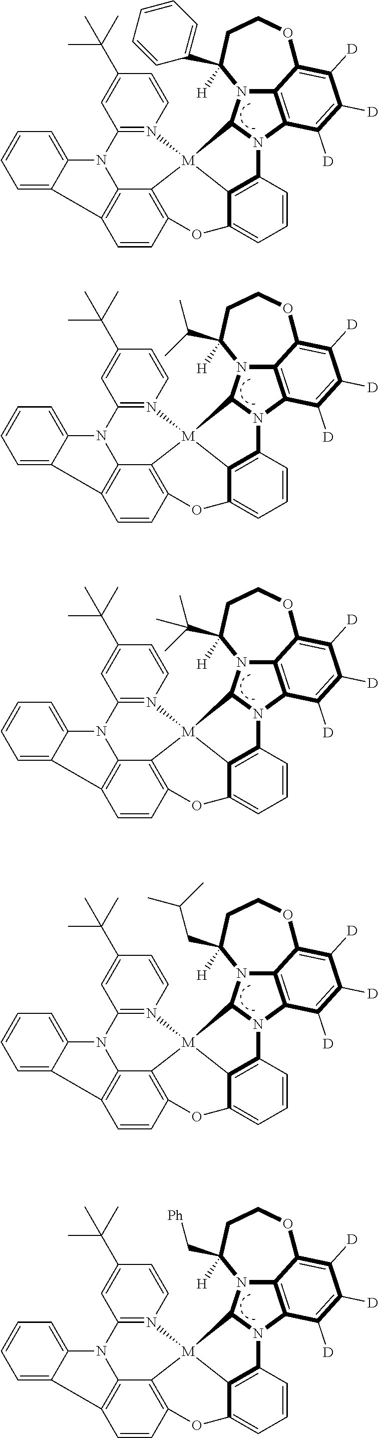

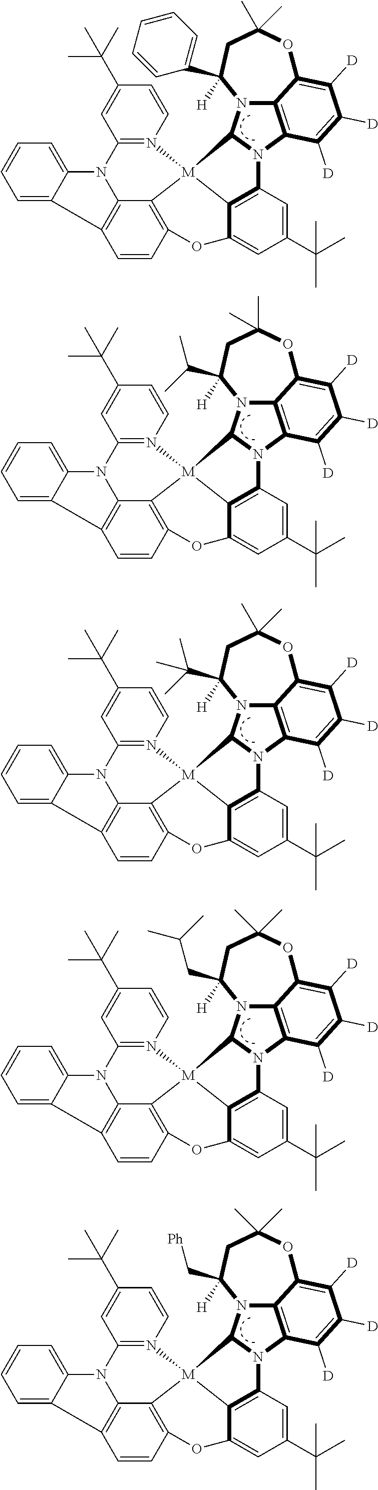

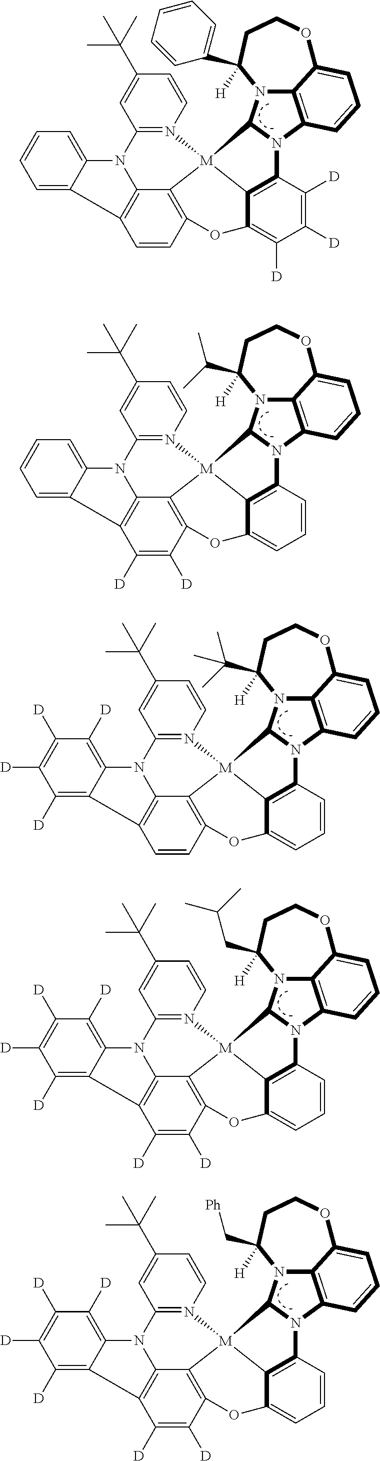

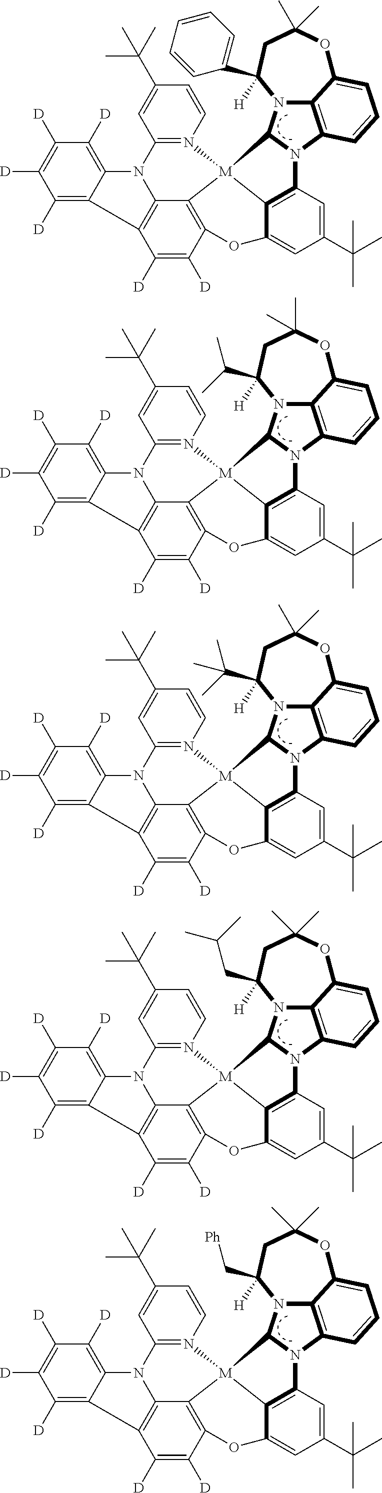

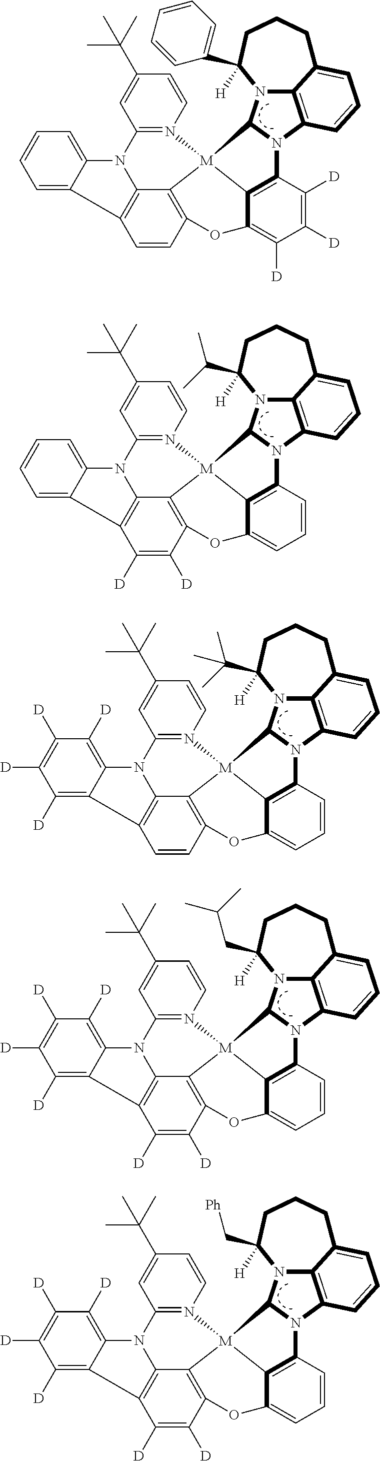

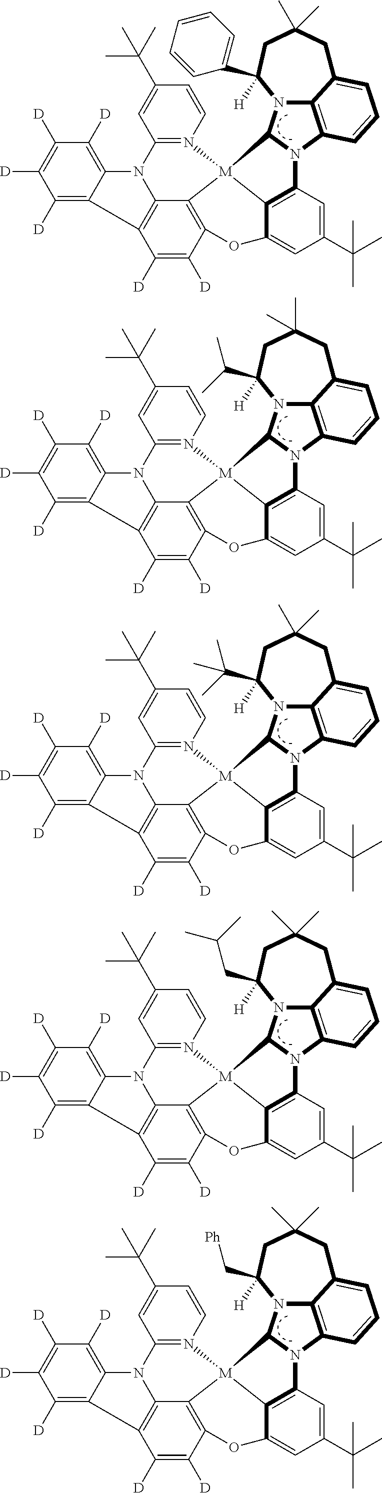

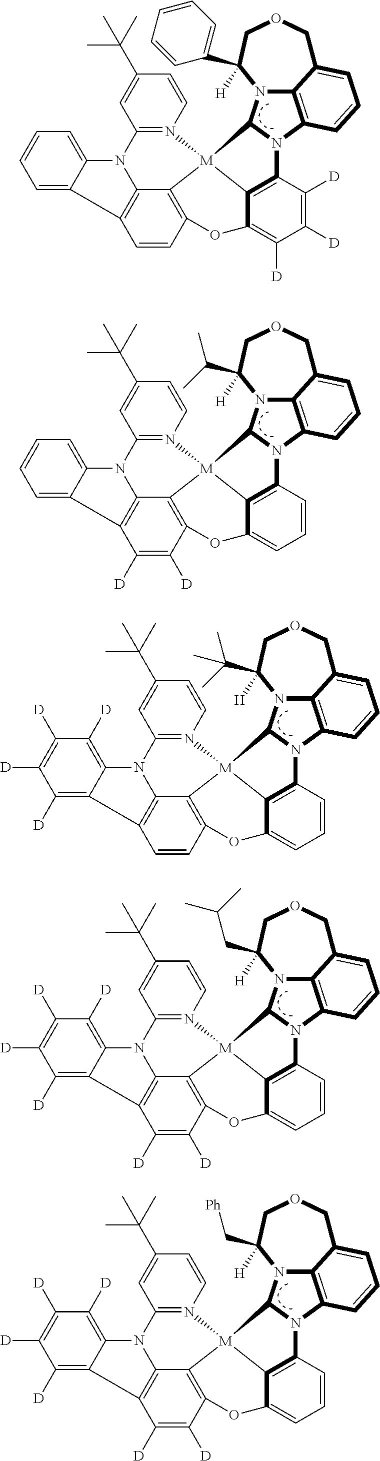

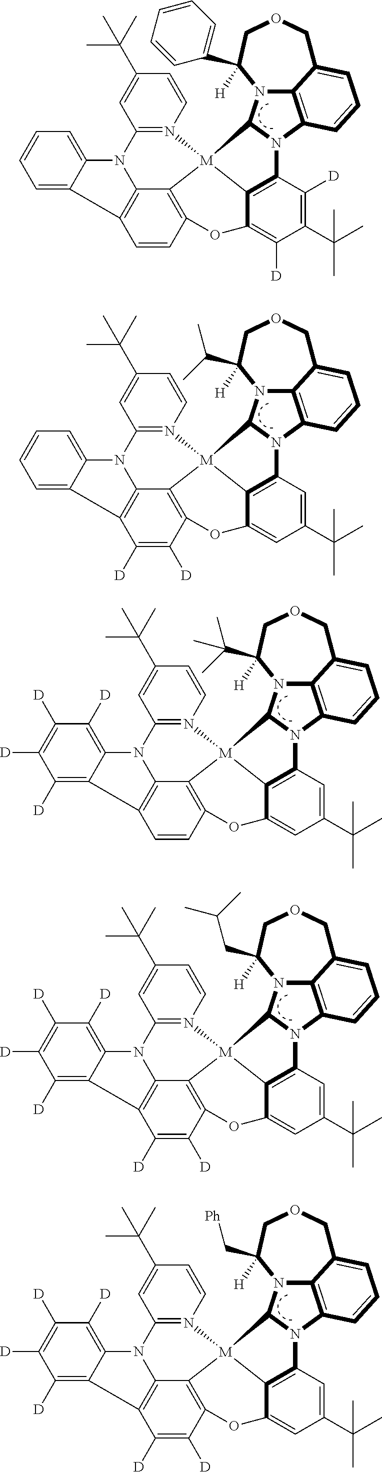

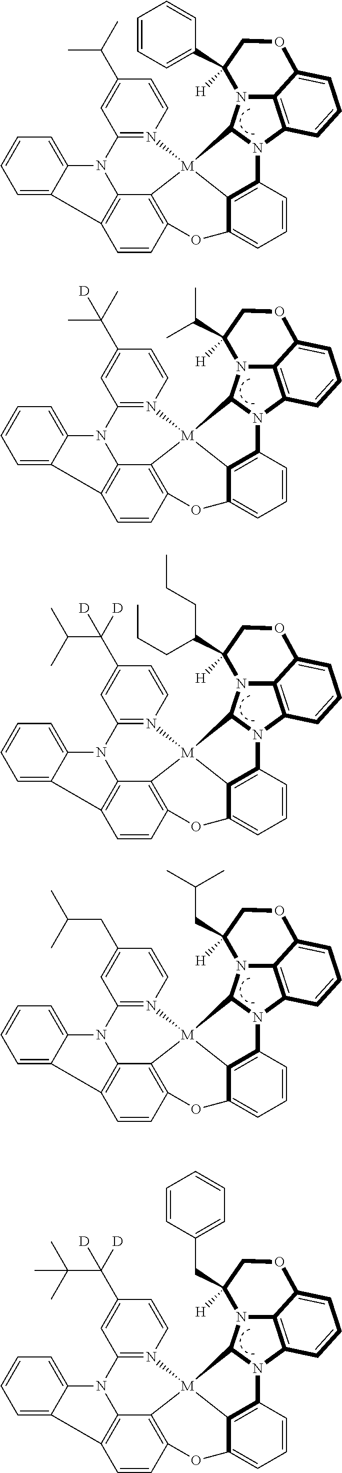

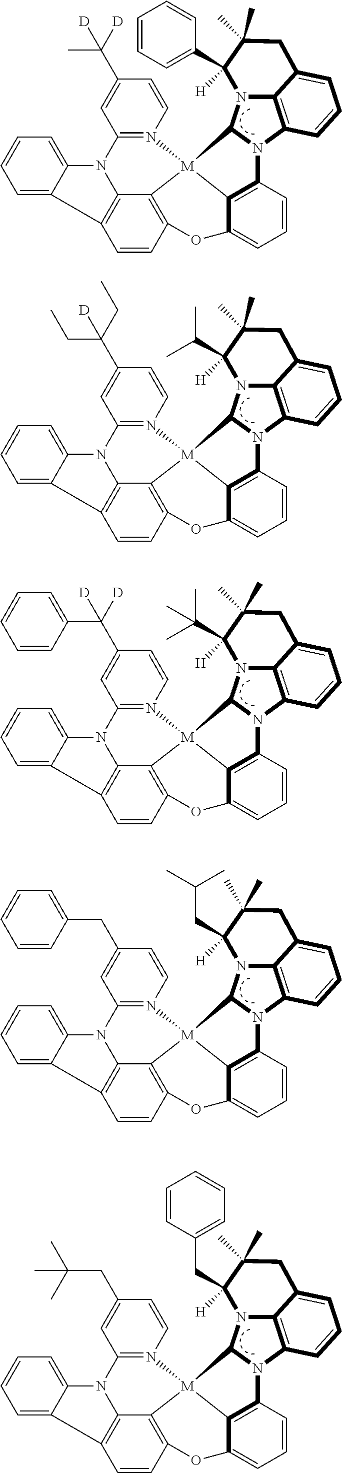

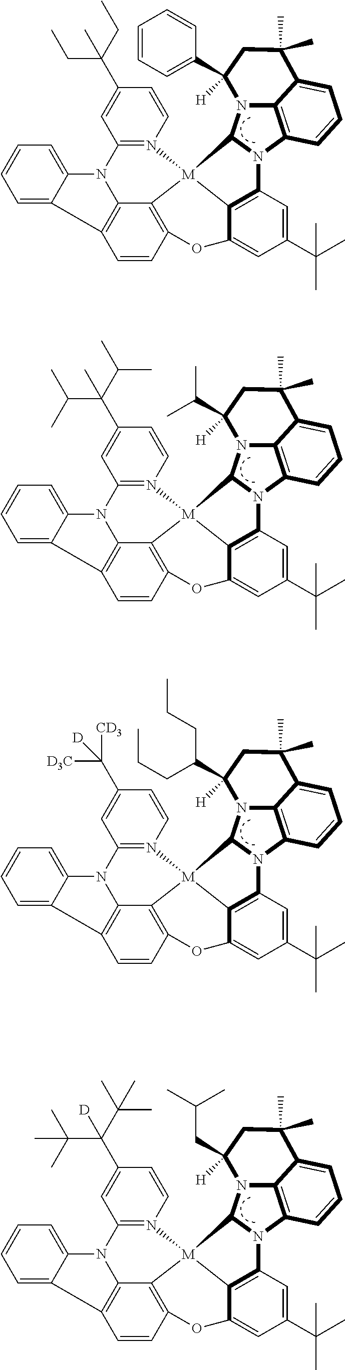

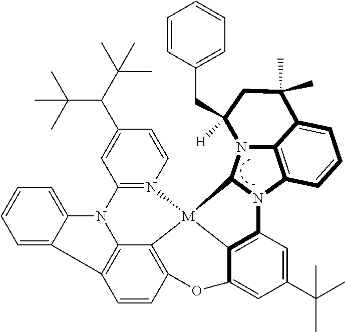

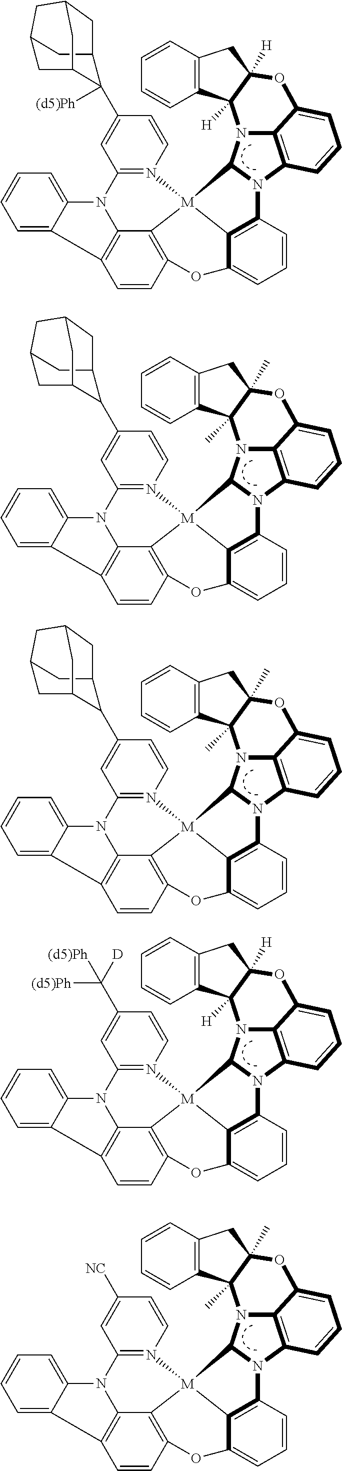

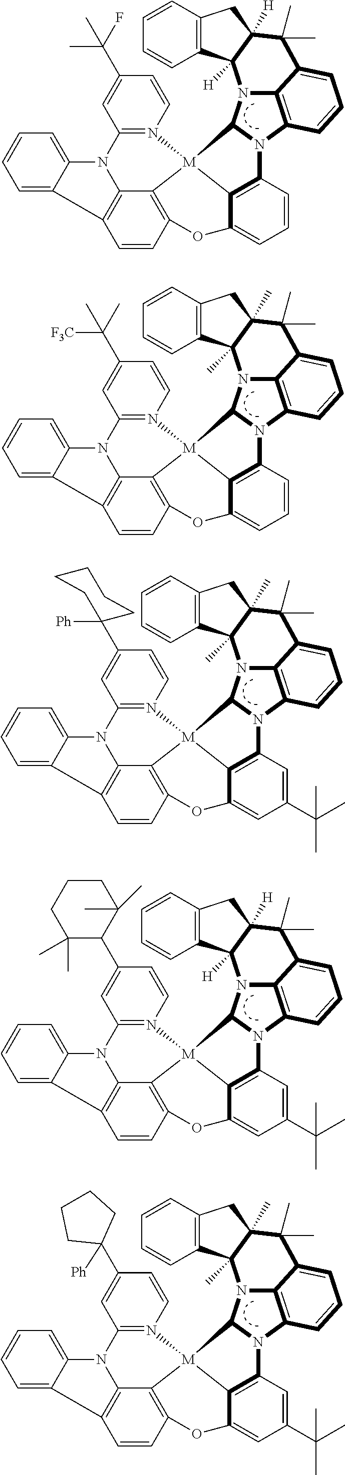

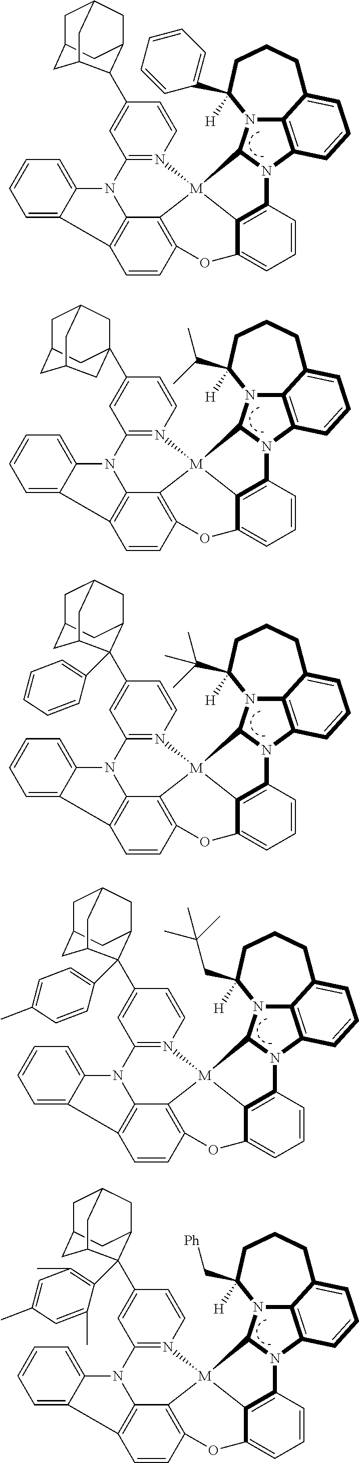

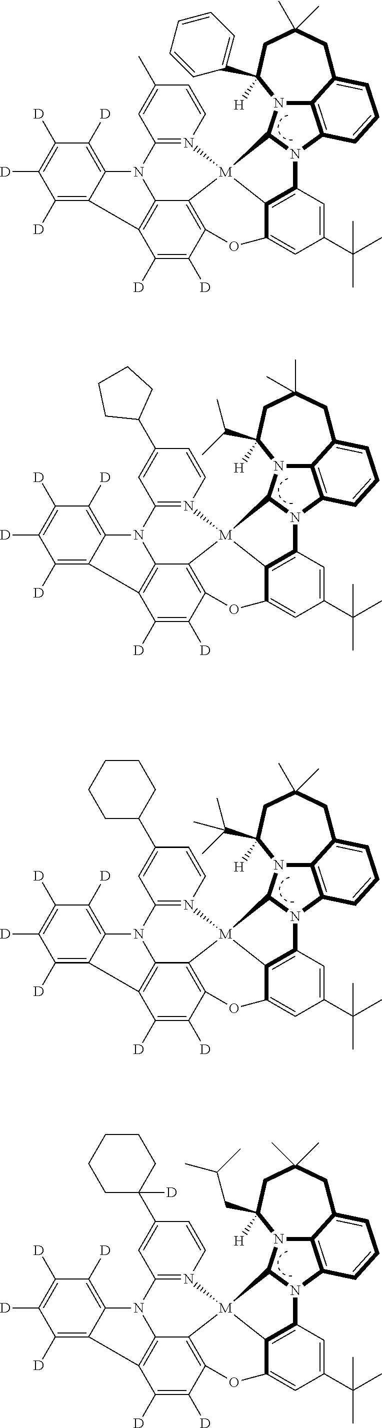

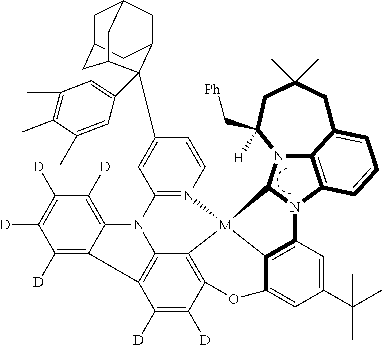

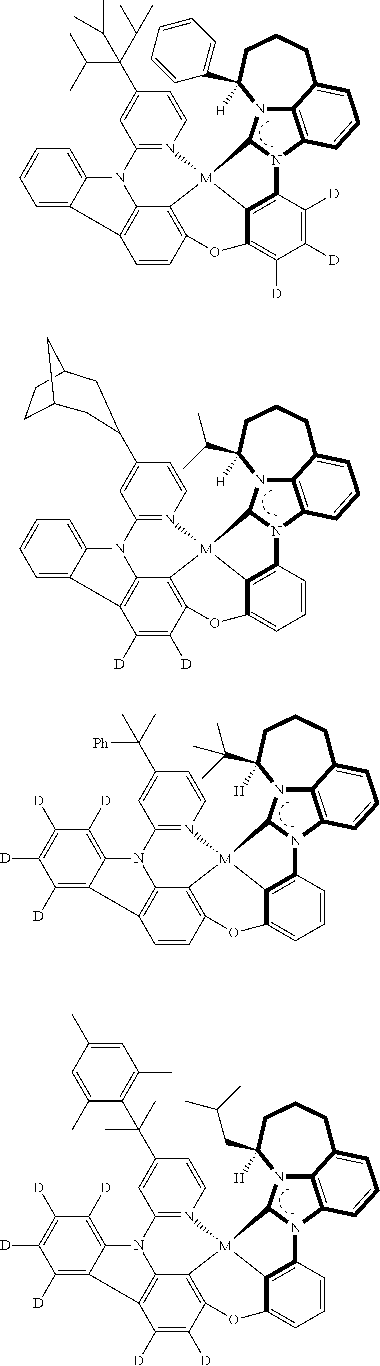

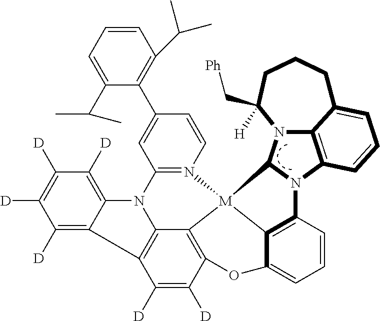

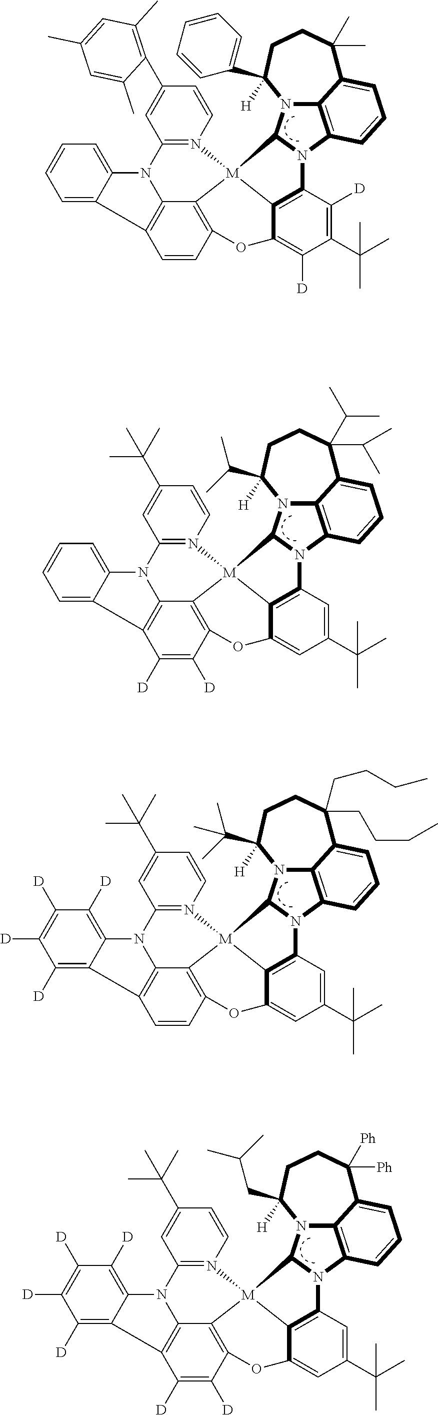

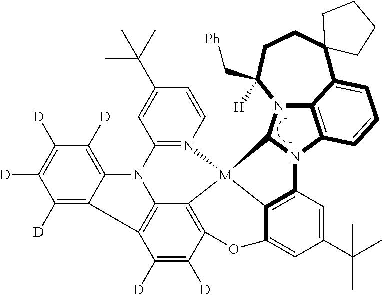

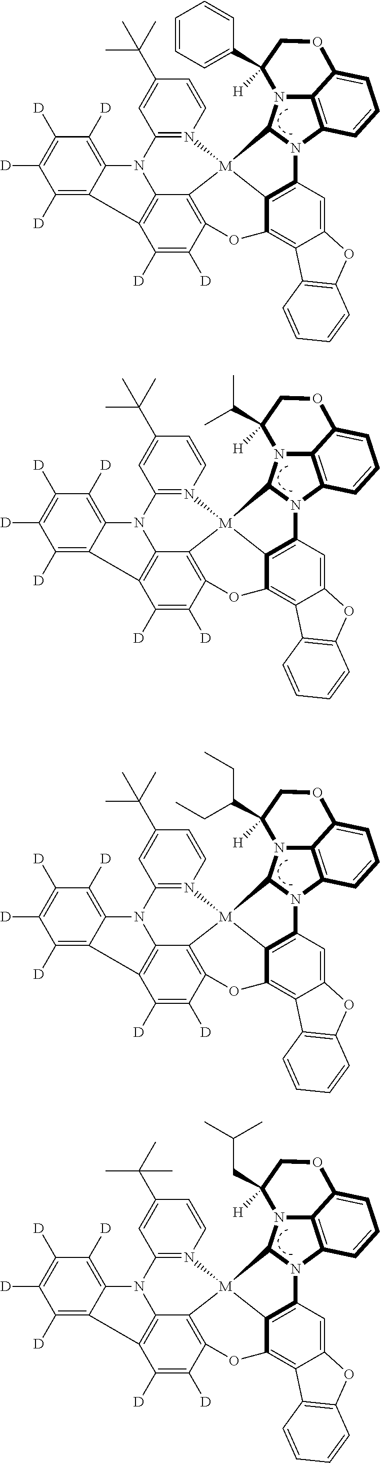

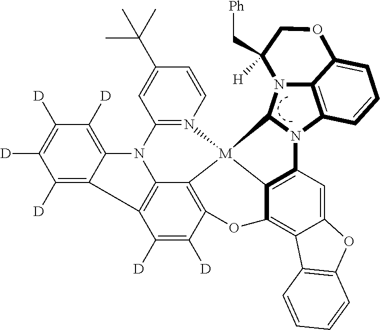

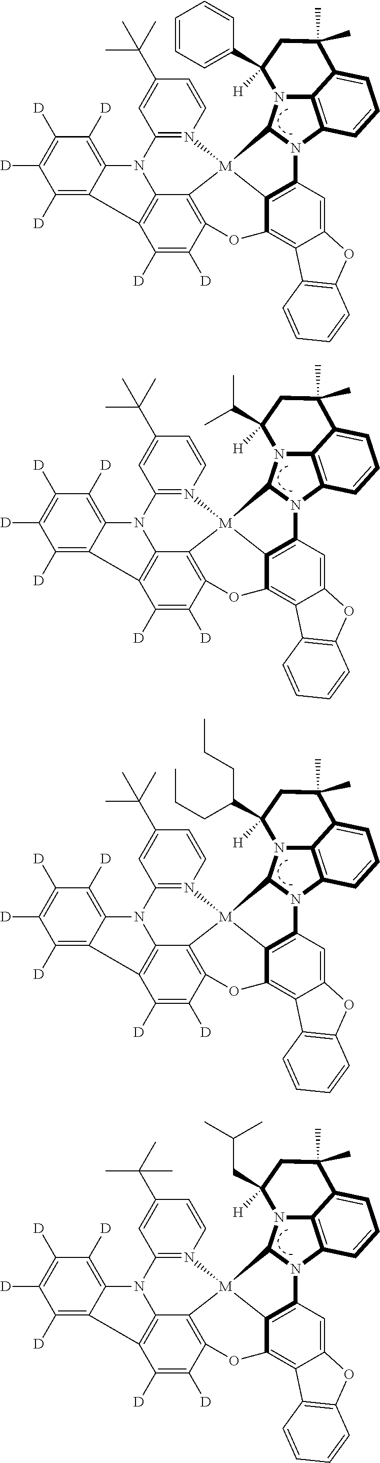

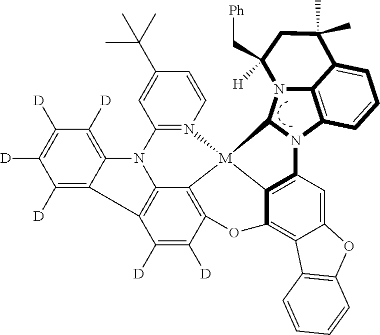

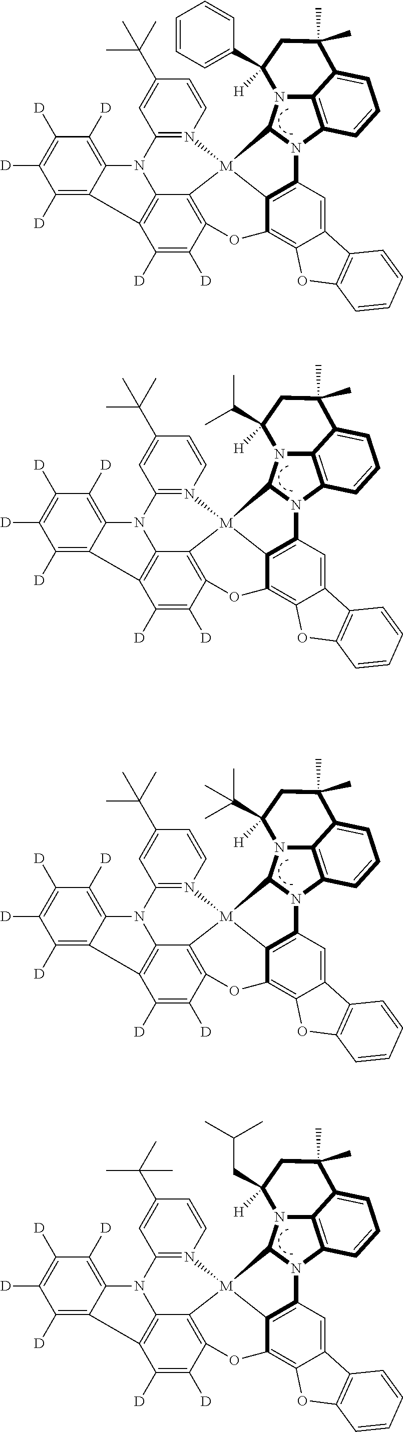

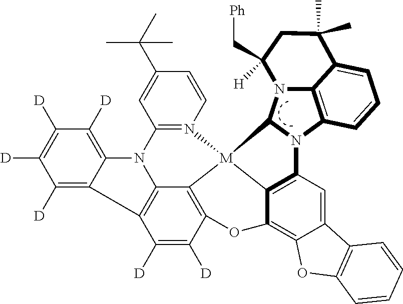

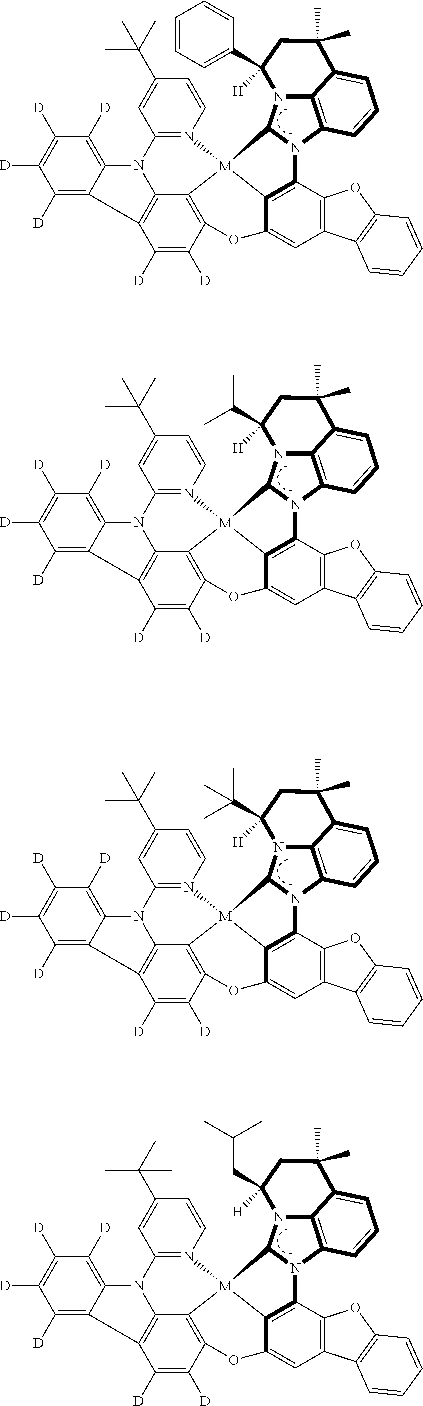

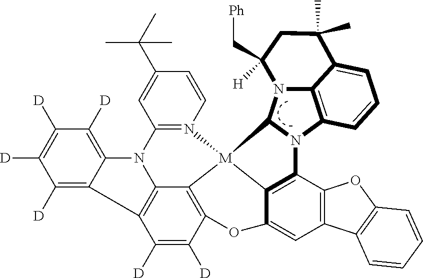

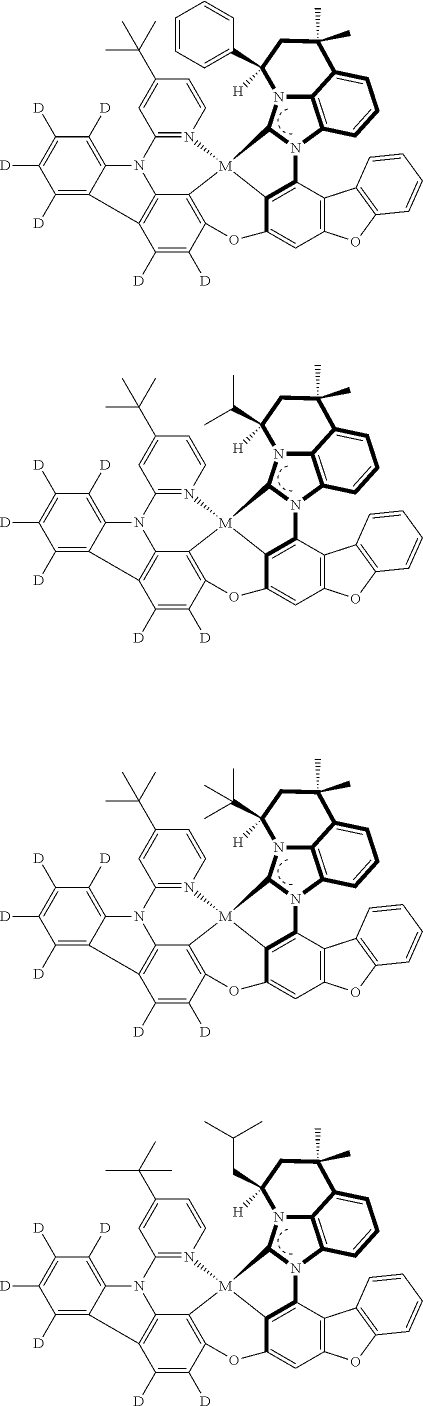

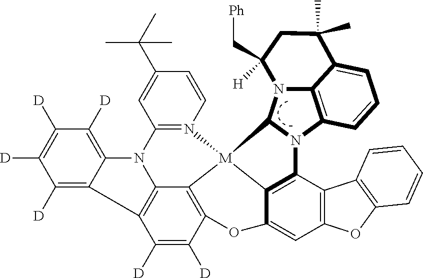

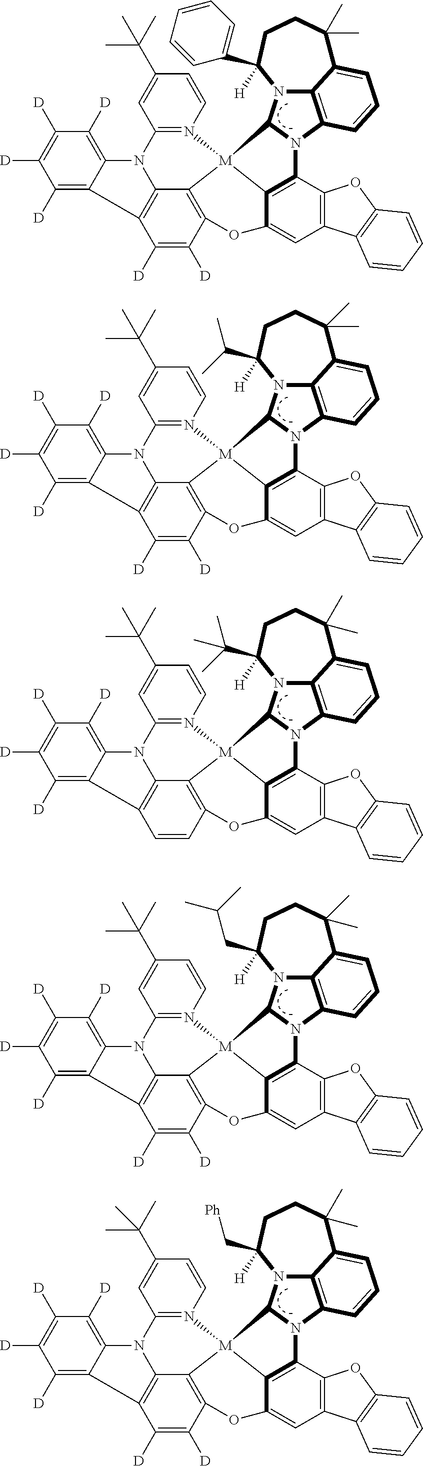

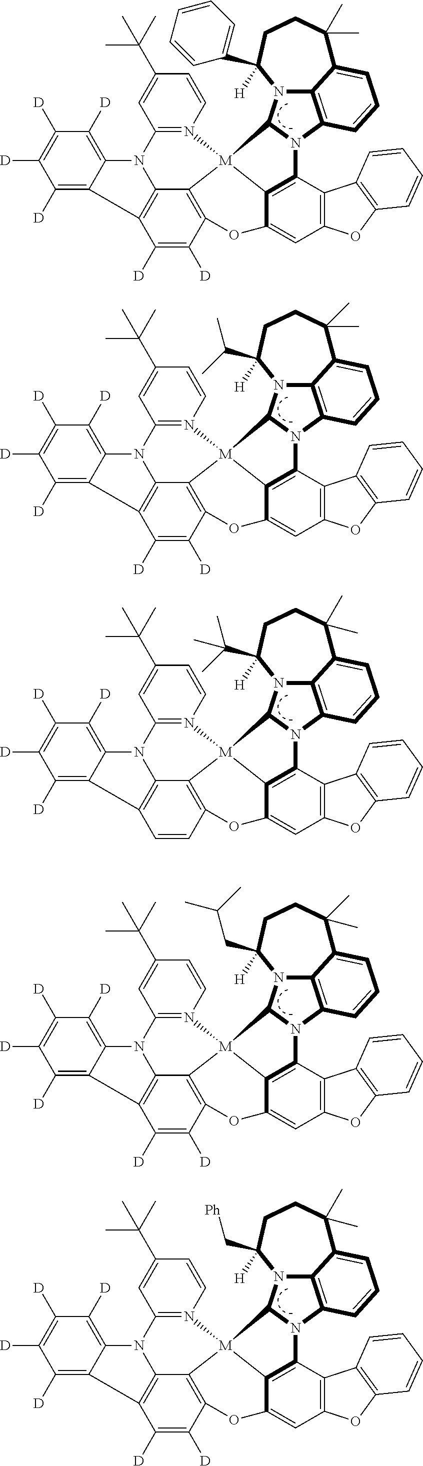

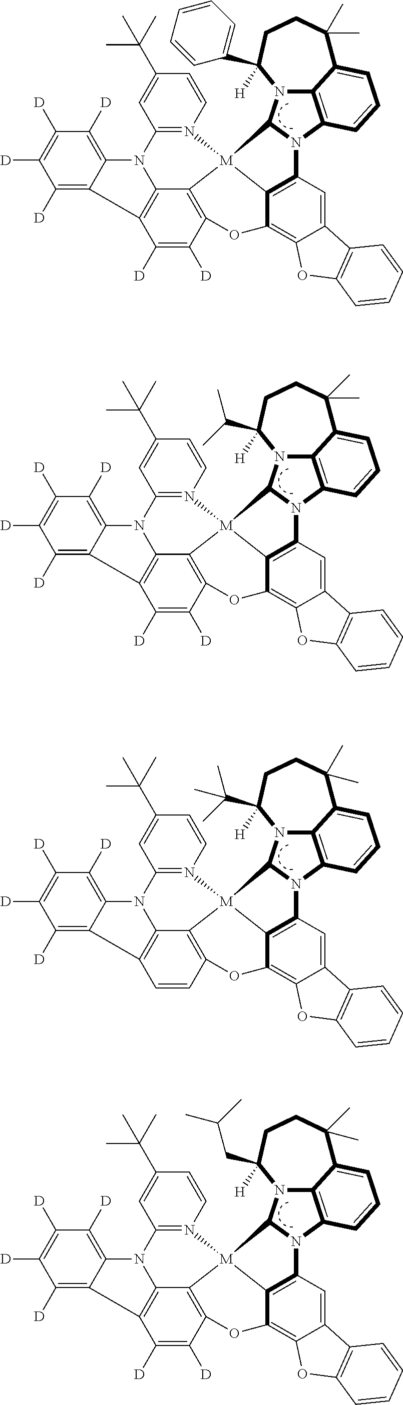

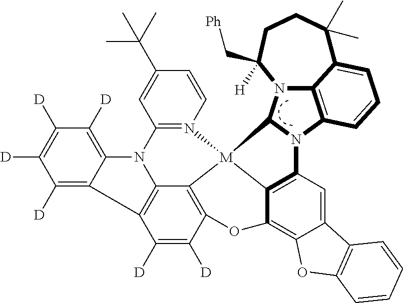

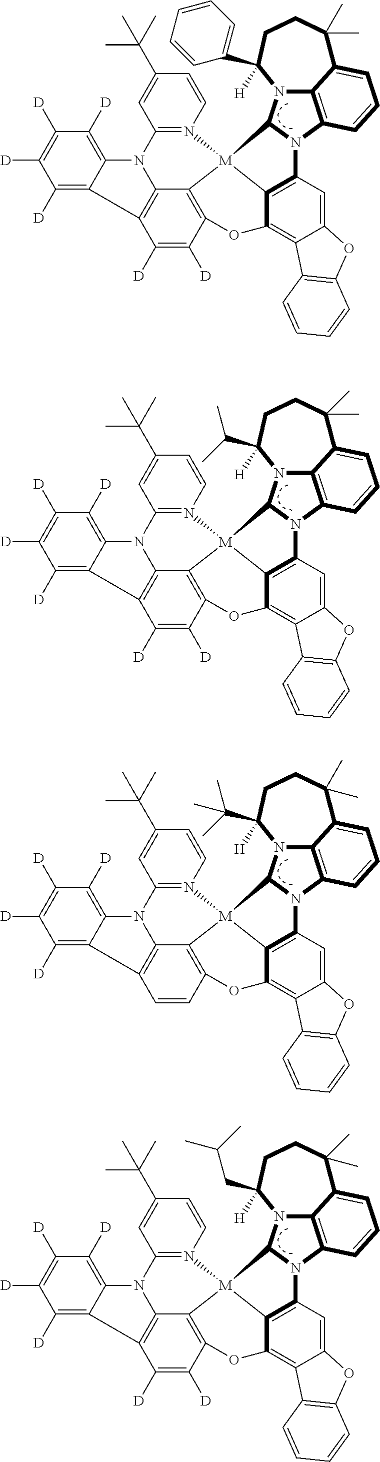

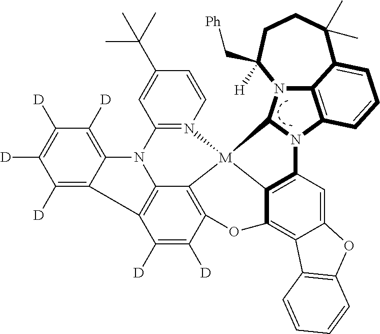

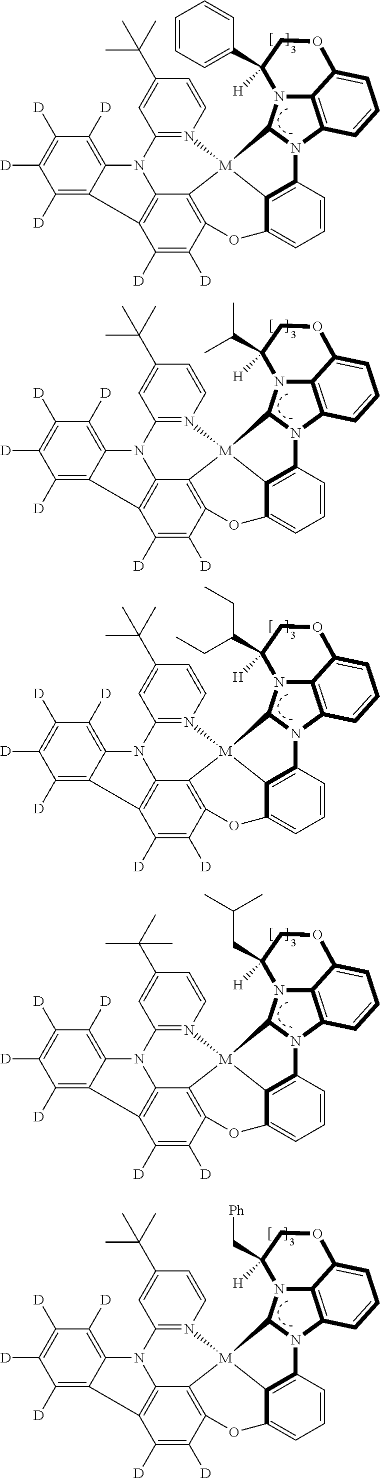

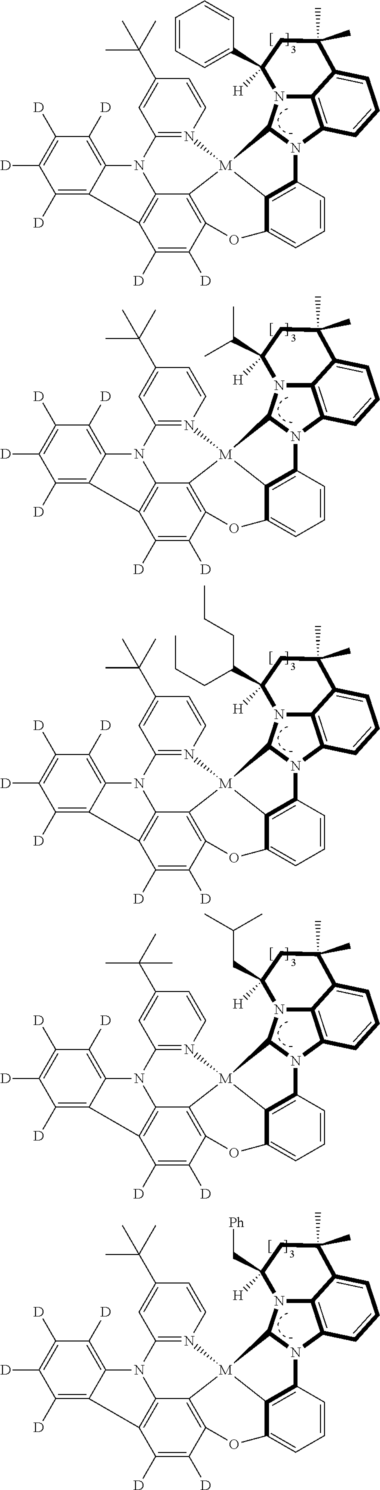

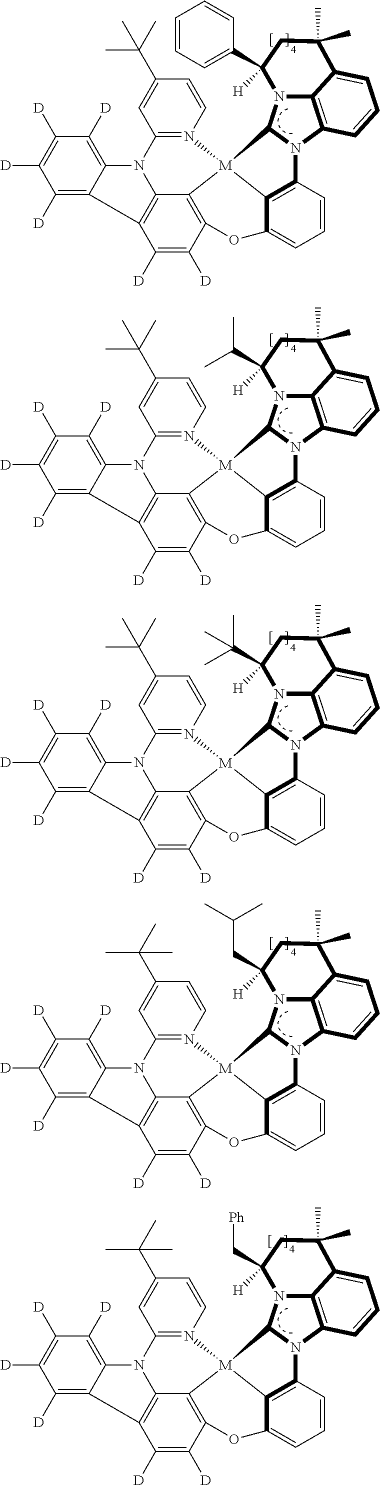

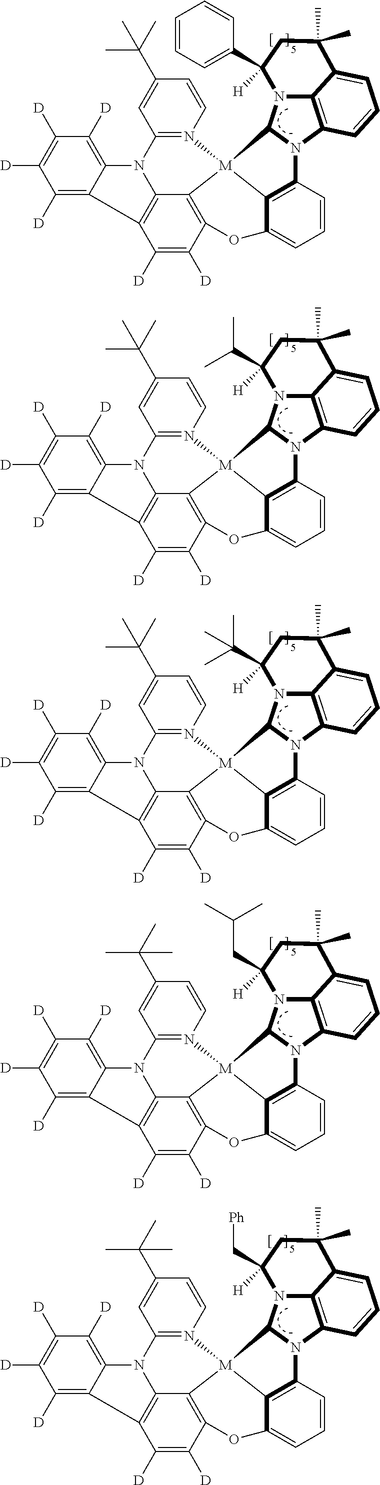

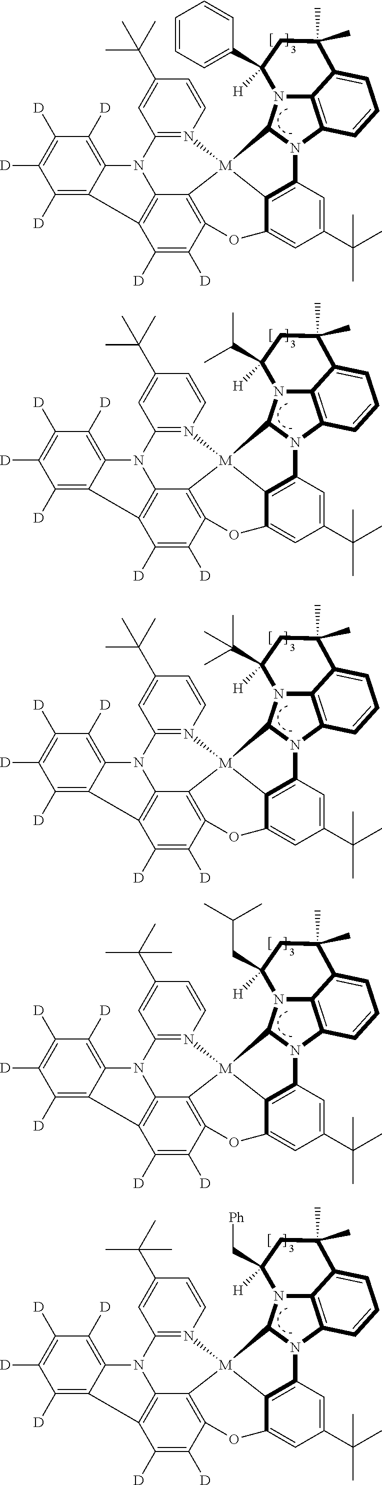

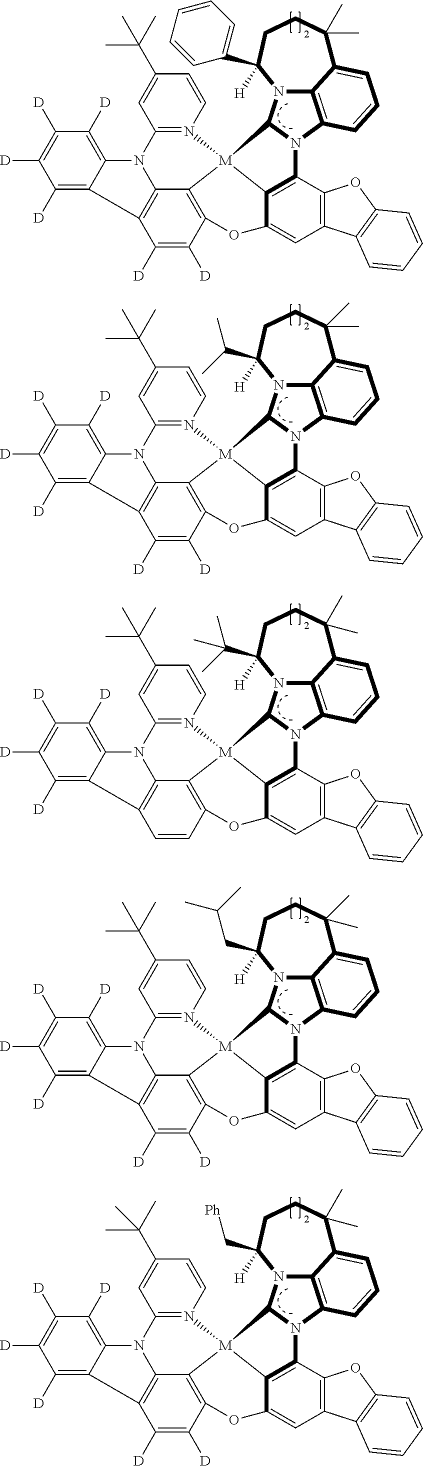

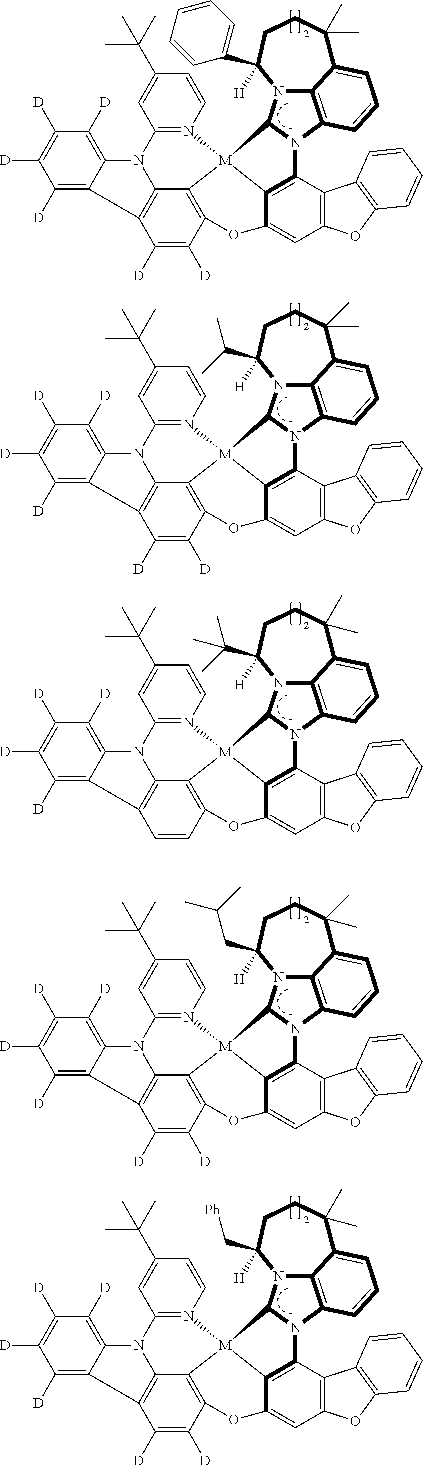

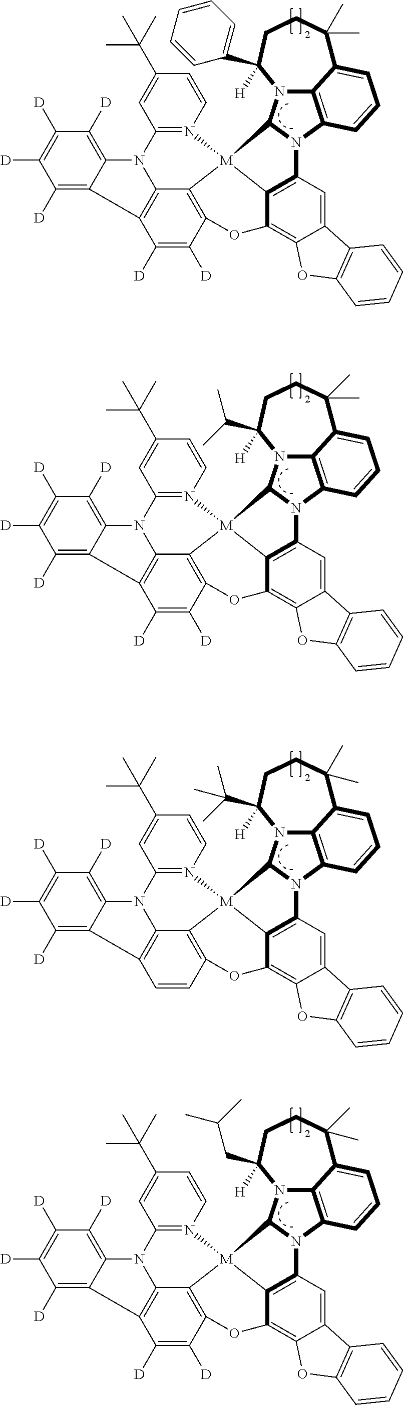

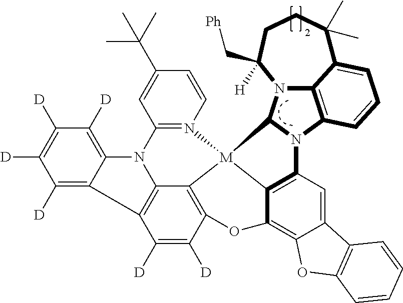

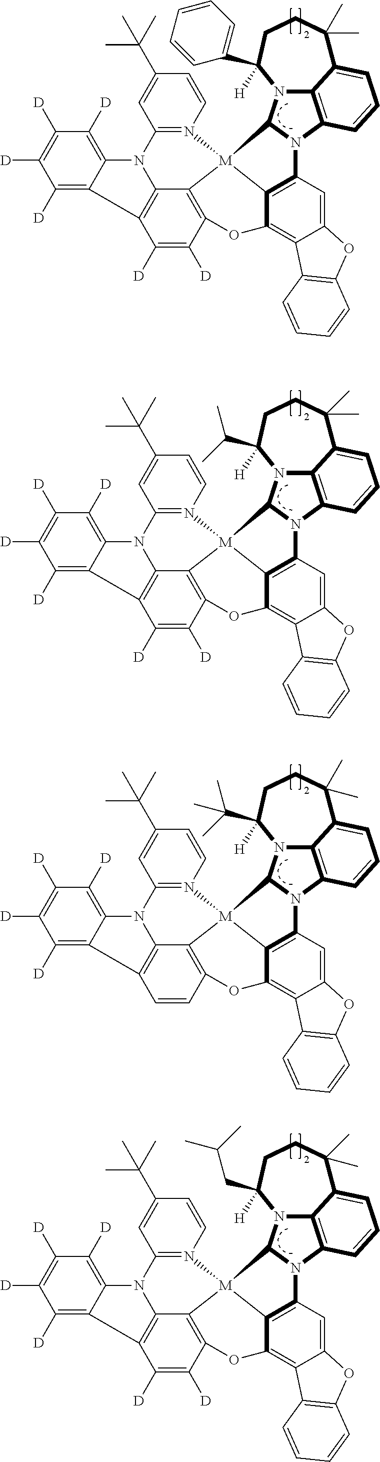

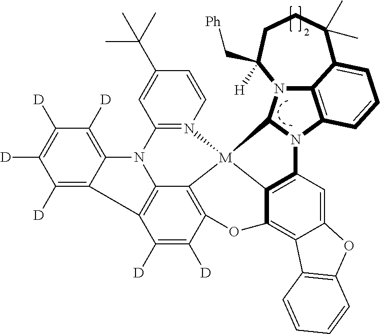

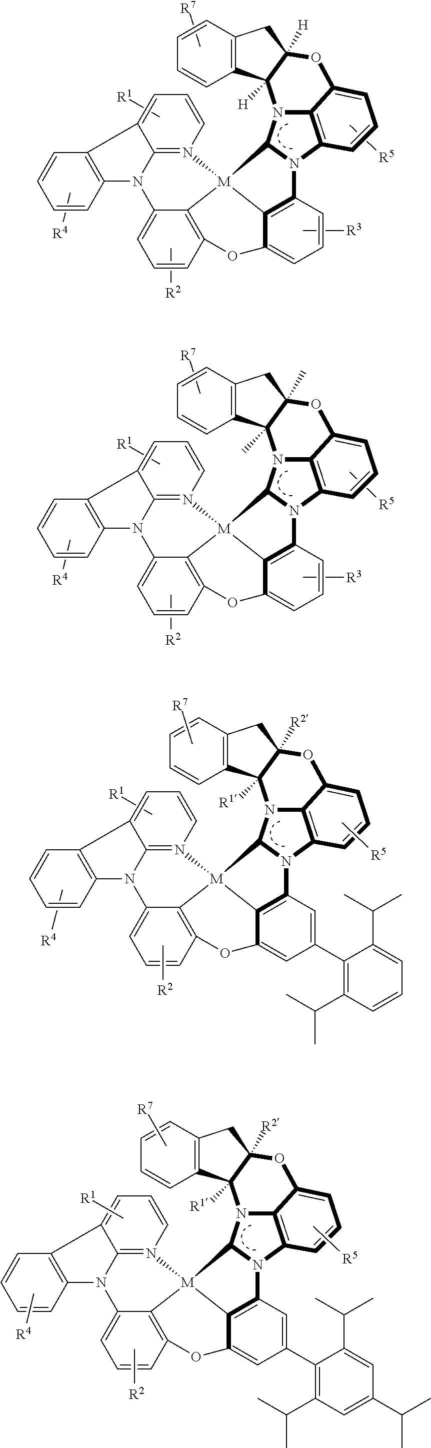

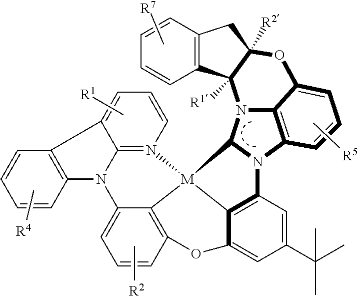

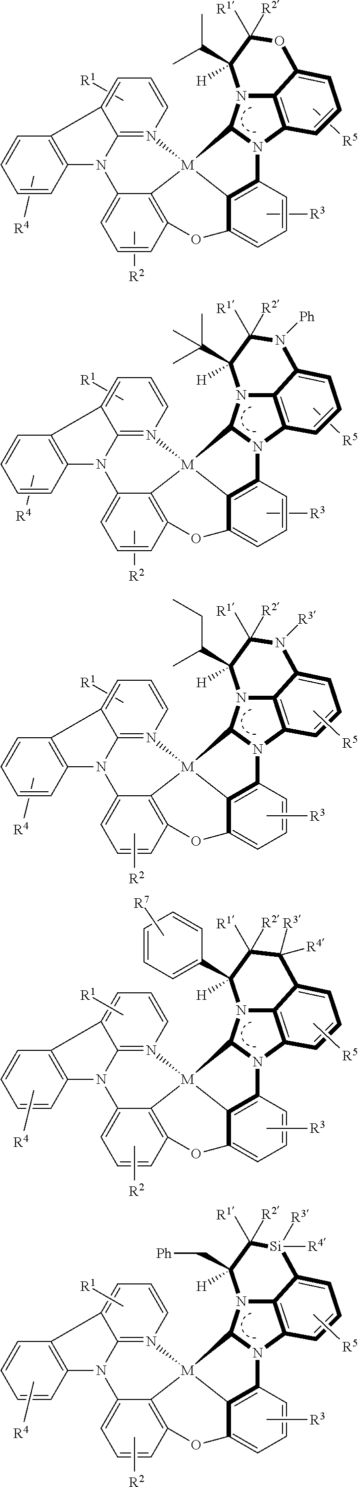

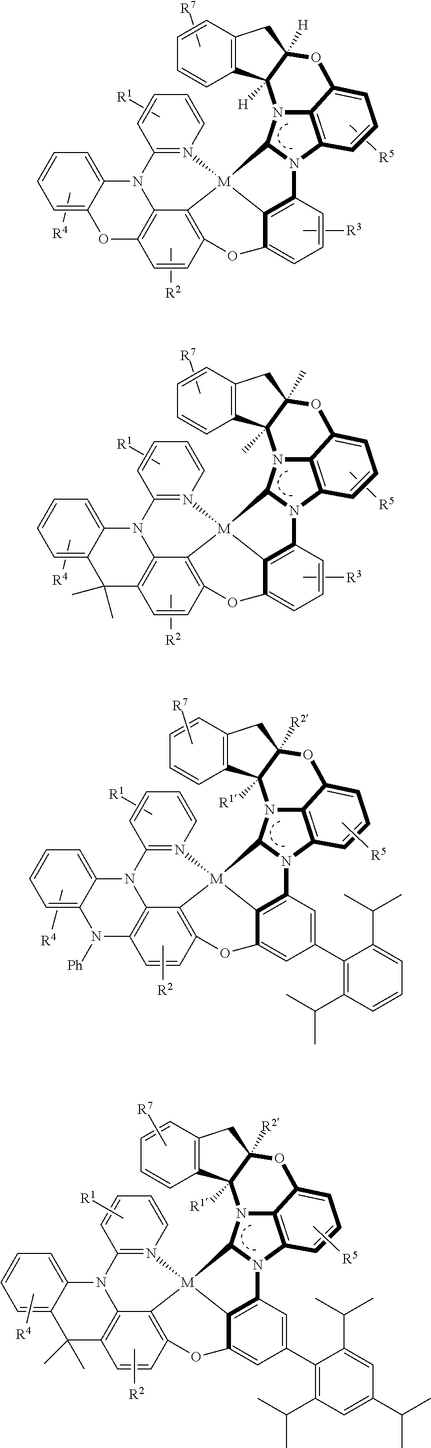

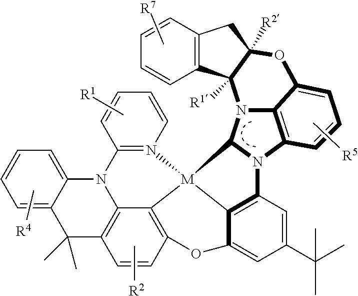

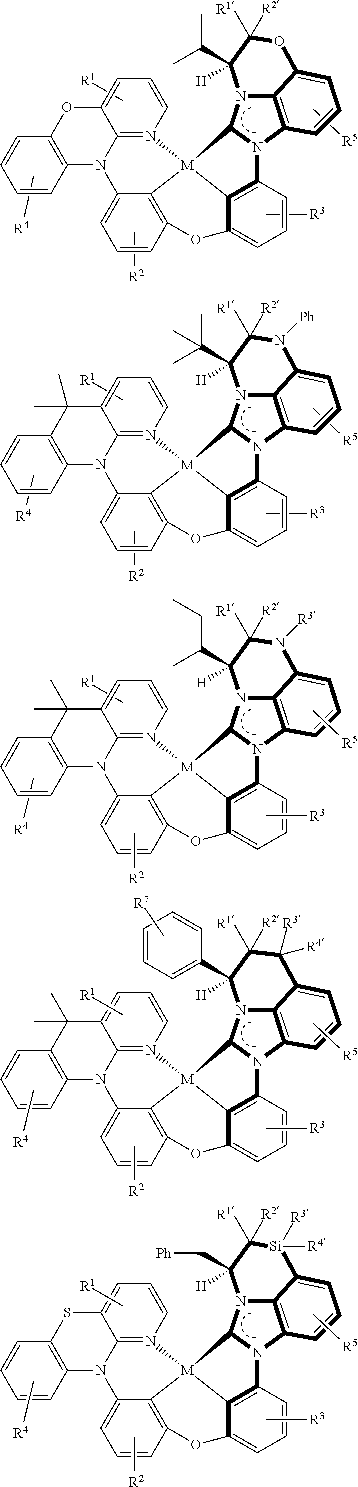

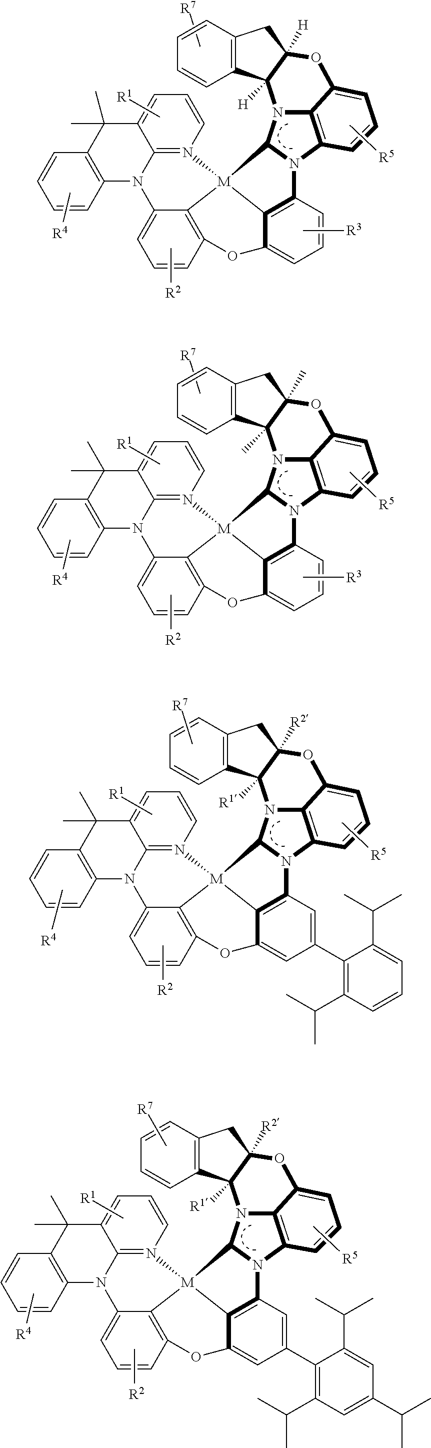

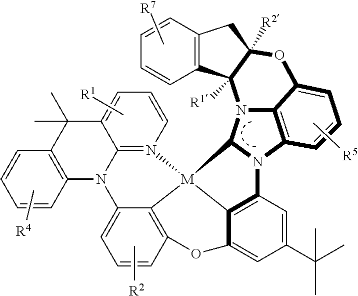

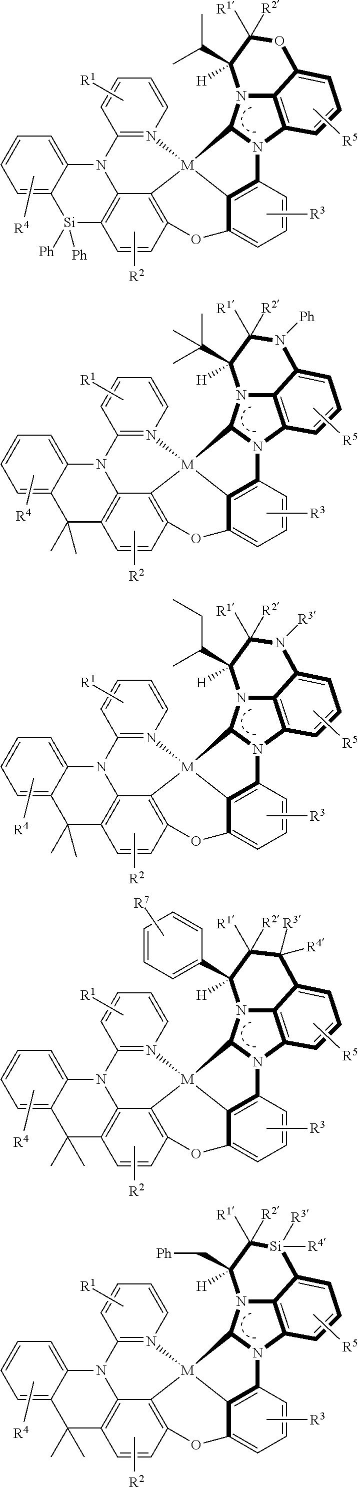

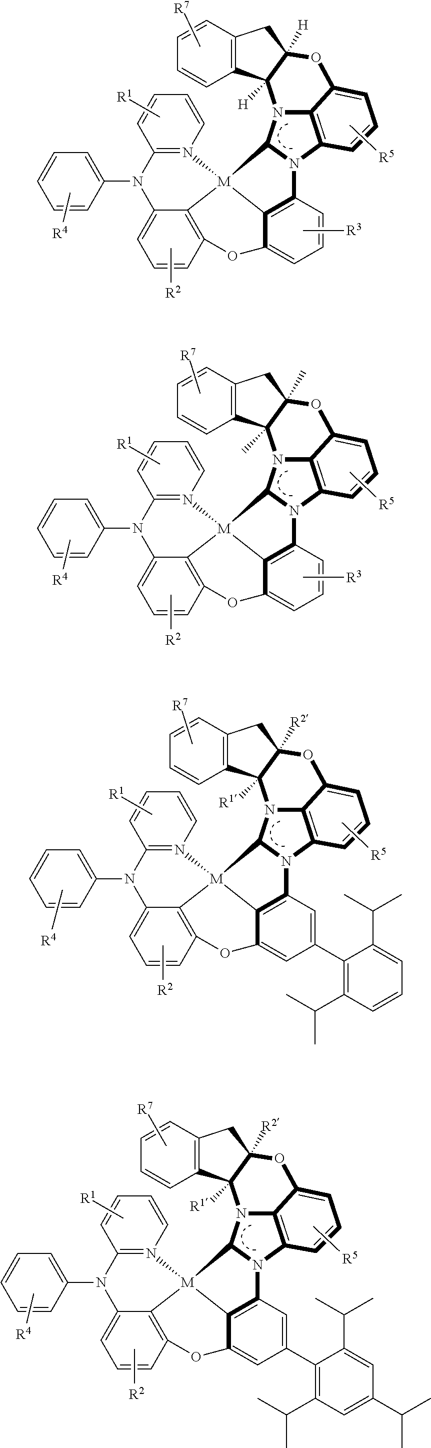

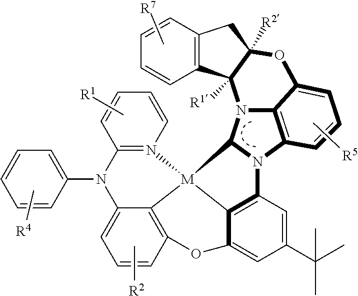

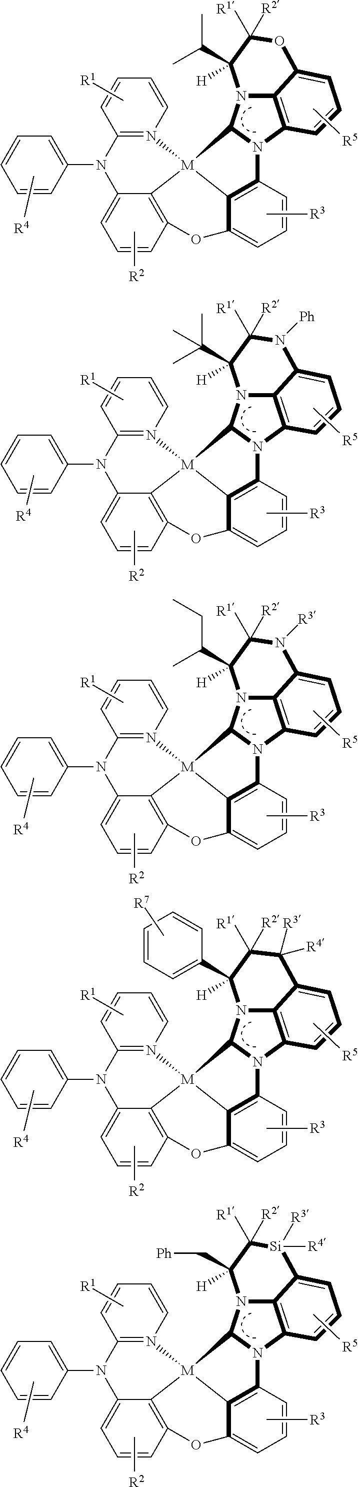

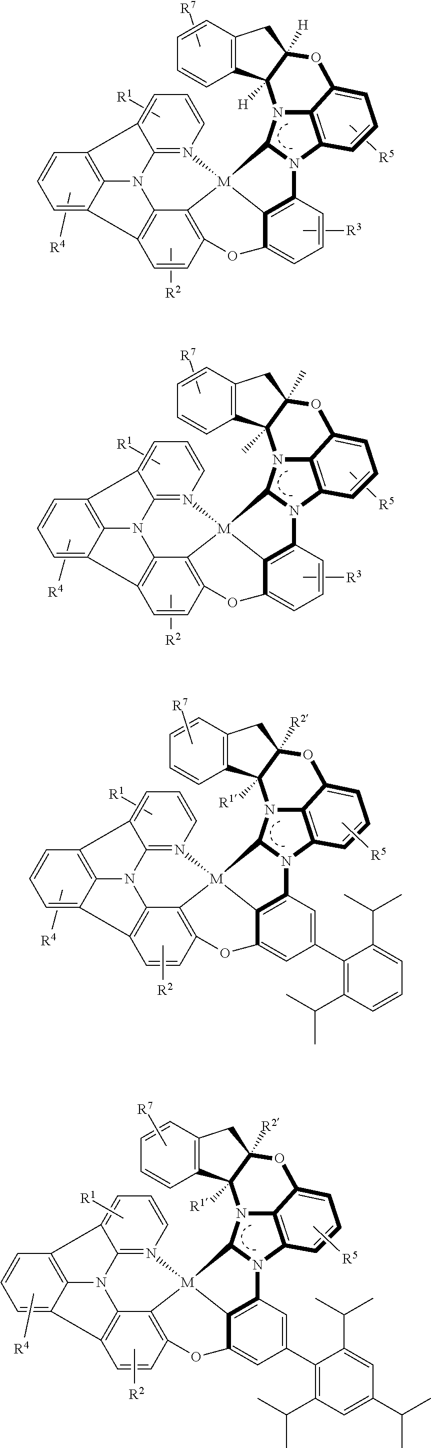

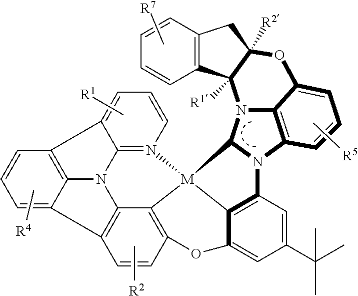

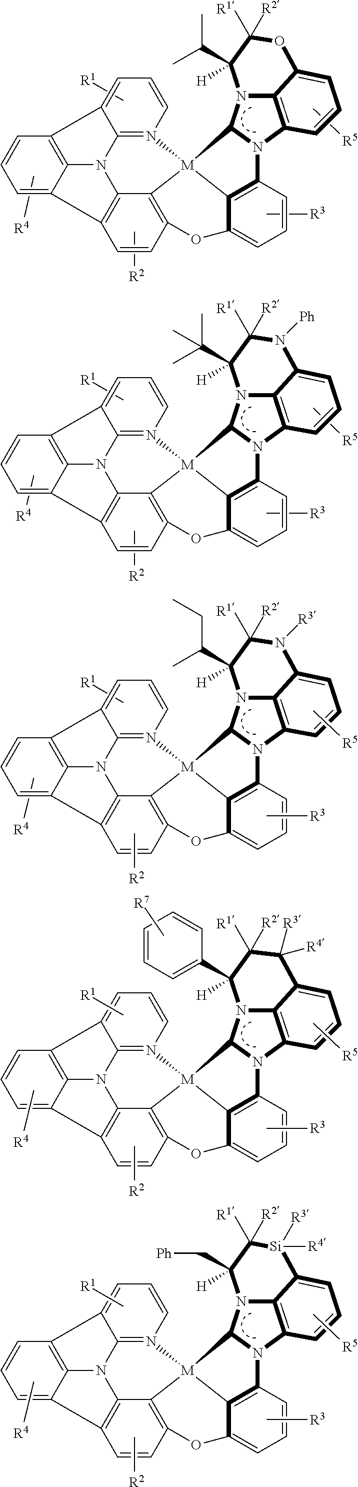

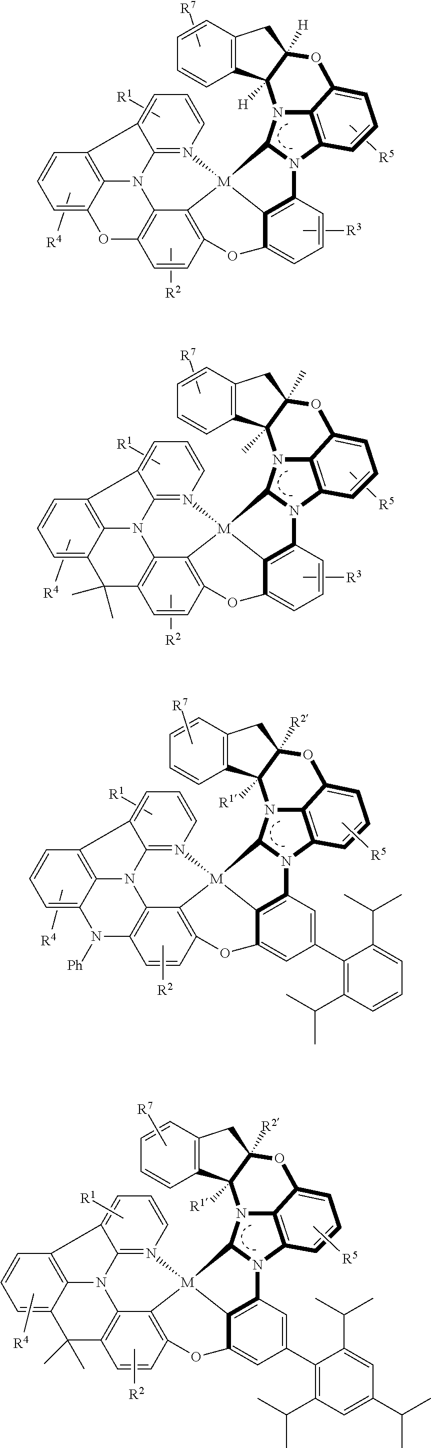

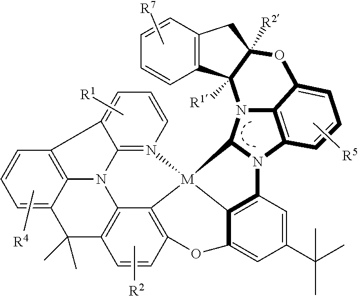

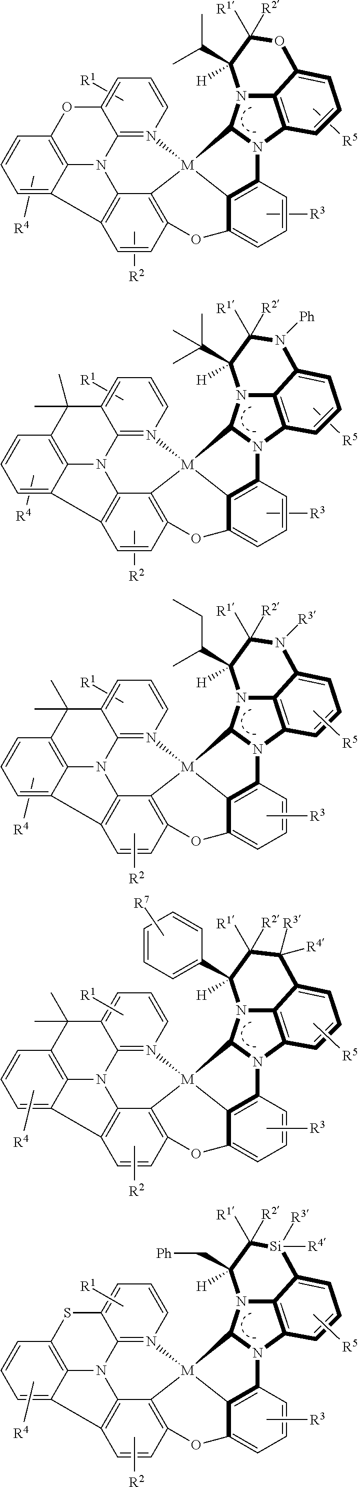

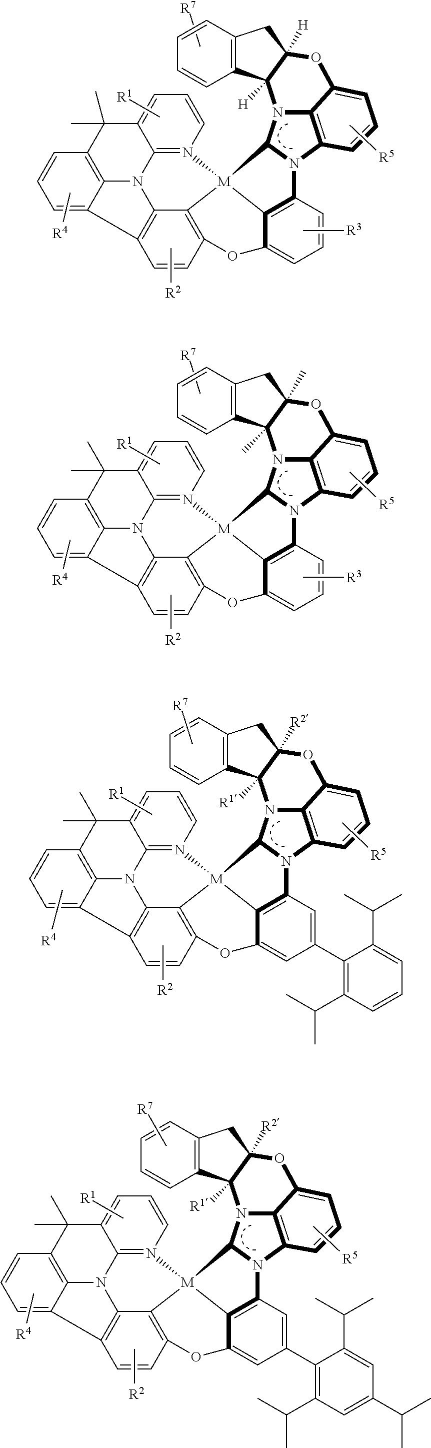

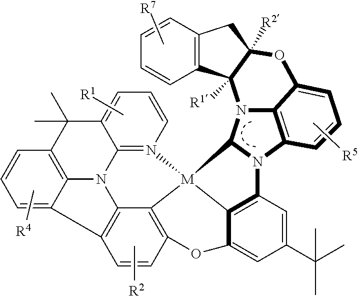

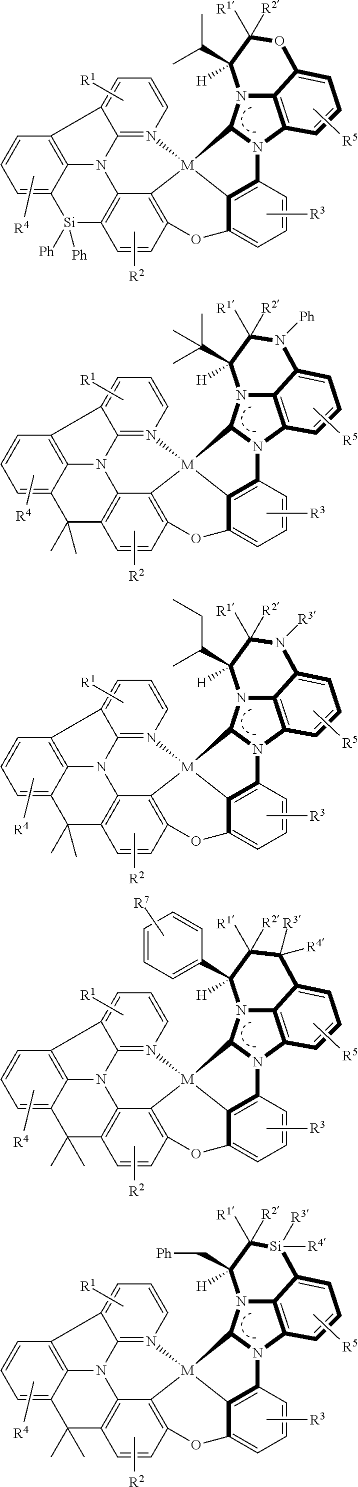

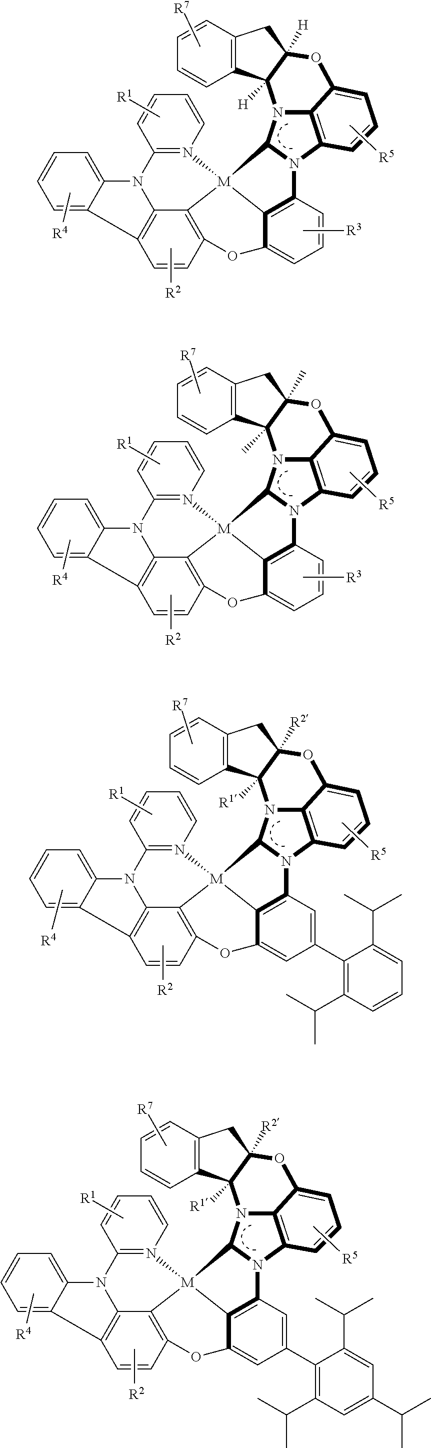

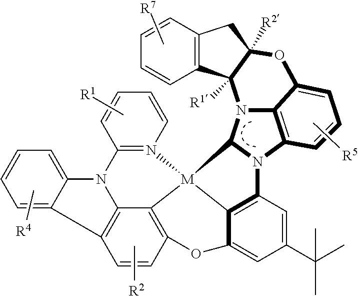

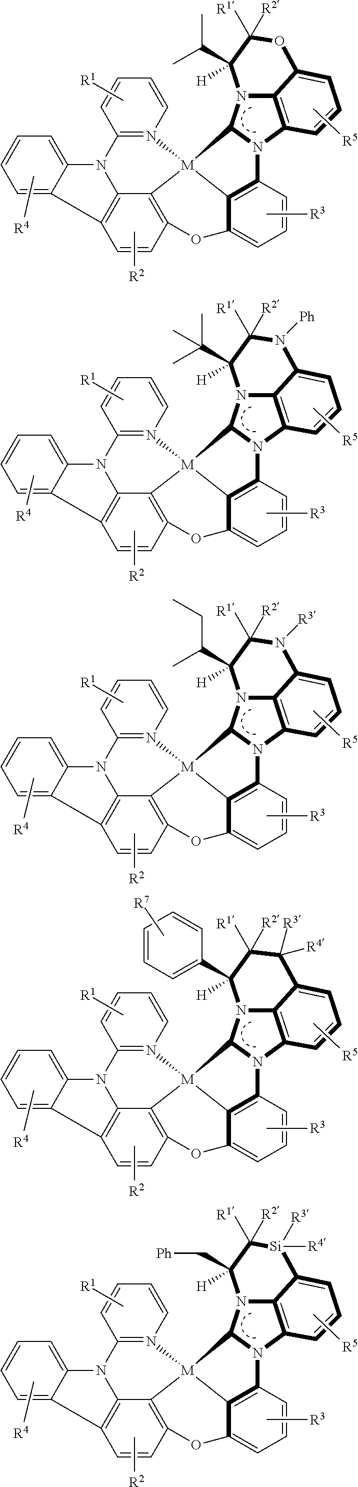

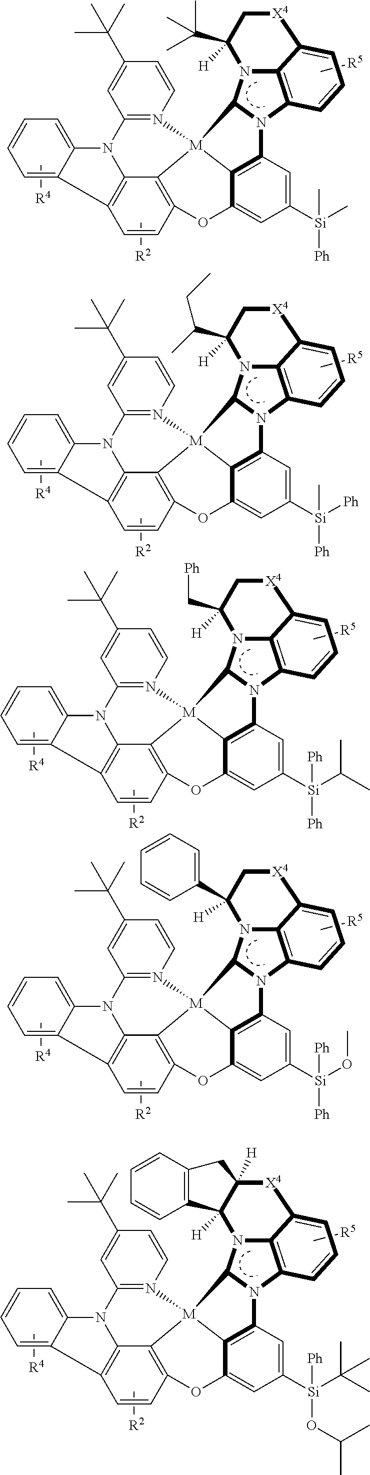

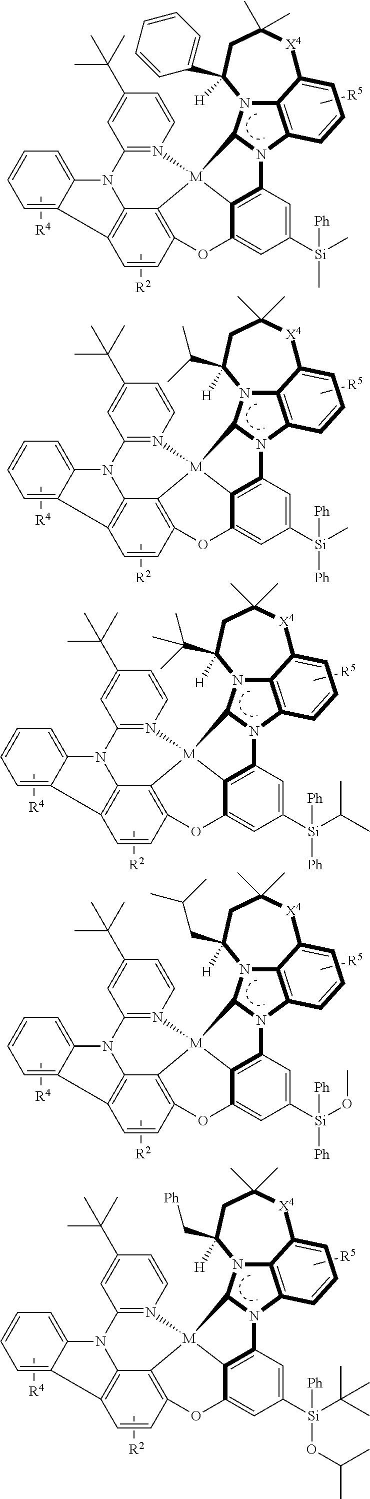

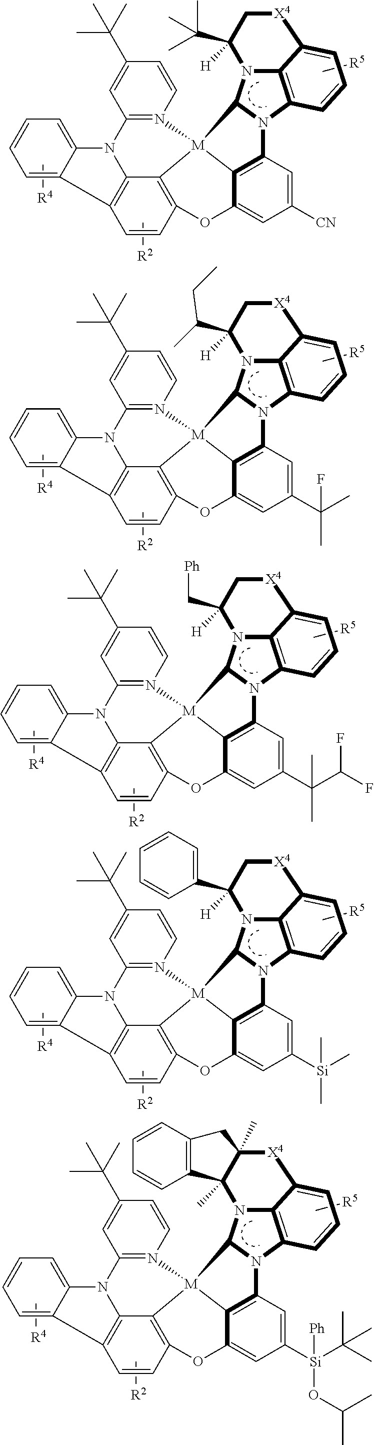

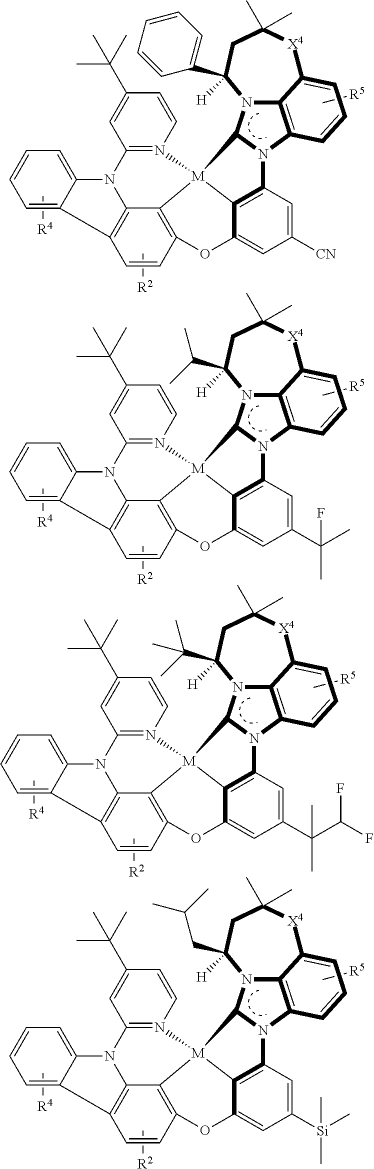

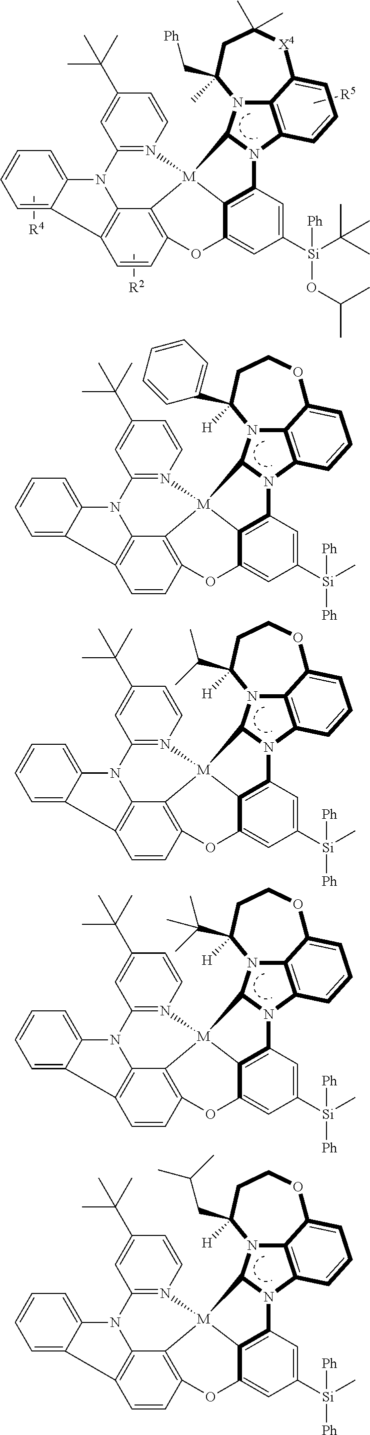

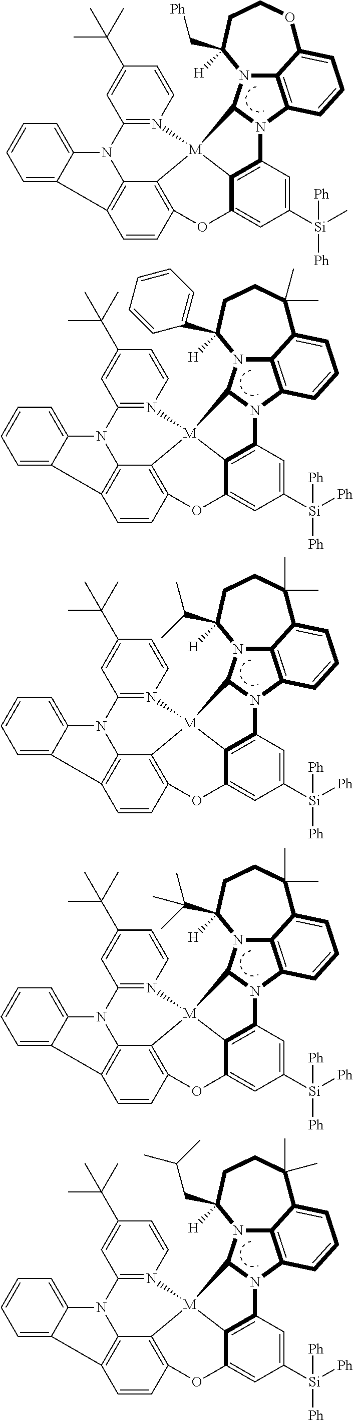

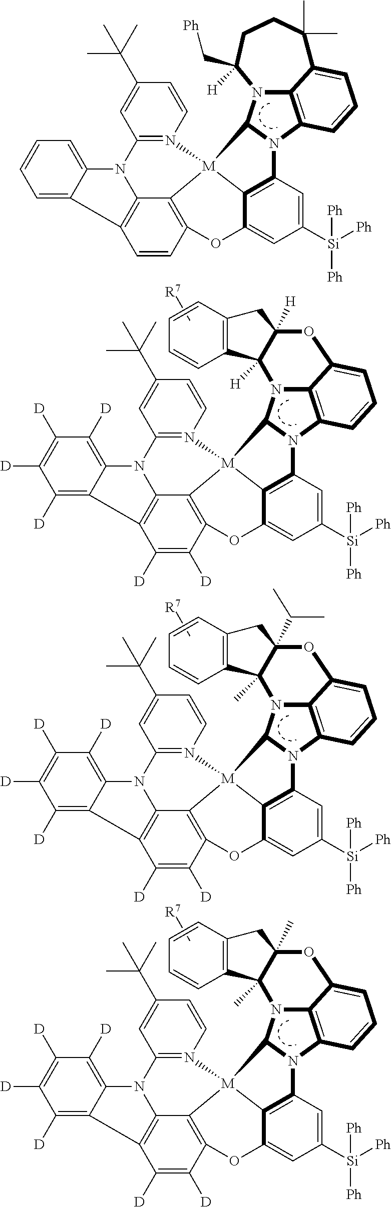

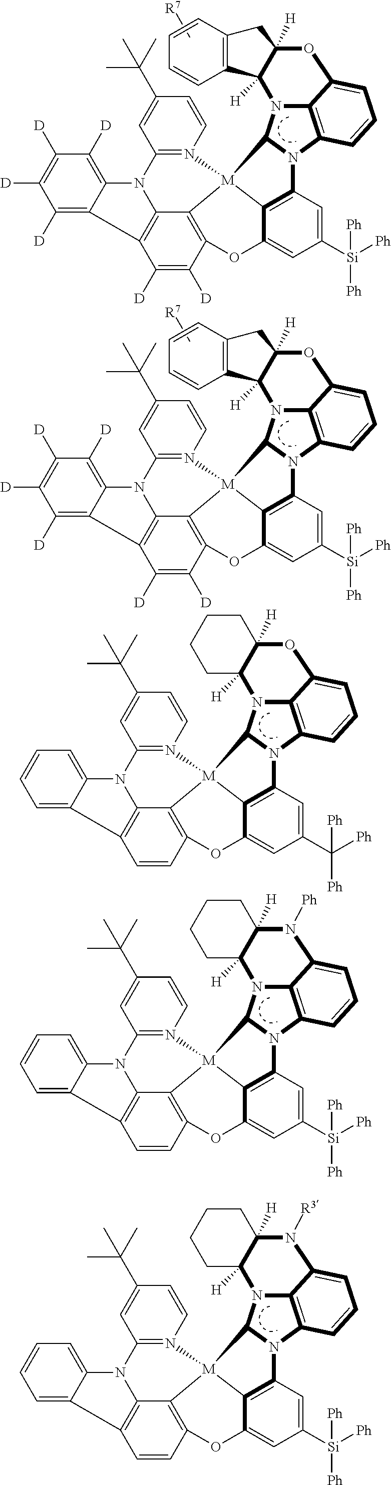

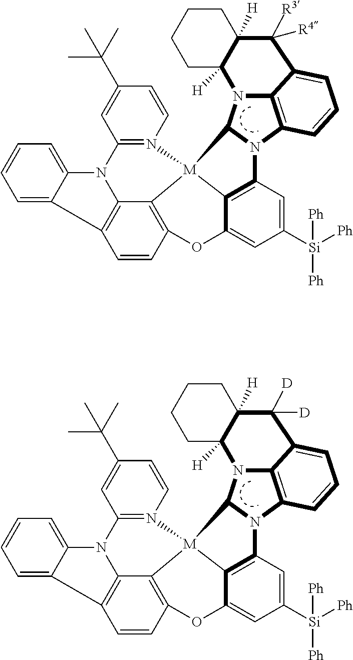

Furthermore, the application of helically chiral tetradentate cyclometalated platinum(II) or palladium(II) complexes circularly polarized luminescent materials based on benzocarbene and its derivative structural units in organic light-emitting devices, 3D display devices, 3D imaging devices, optical information encryption devices, information storage devices, and biological imaging devices.

Furthermore, the organic light-emitting elements are organic light-emitting diodes, light-emitting diodes, or luminescent electrochemical cells.

Furthermore, the light-emitting element comprises a first electrode, a second electrode, and an organic layer arranged between the first electrode and the second electrode. The organic layer at least comprises a helically chiral tetradentate cyclometalated platinum(II) or palladium(II) complex circularly polarized light-emitting material based on a benzocarbene and its derivative structural unit.

The beneficial effects of the present disclosure are follows:
(1) Central chirality autonomously induced spiral chirality: By designing and developing a tetradentate ligand with a central chiral benzocarbene and its derivative structural fragment L⁵, utilizing the steric hindrance effect between it and the other end ligand L¹, making the entire tetradentate cyclometalated platinum(II) and palladium(II) complexes be twisted quadrilateral configurations; meanwhile, the entire tetradentate ligands can autonomously coordinate with metal ions in a low steric hindrance manner induced by the central chiral fragment in the tetradentate ligands, which autonomously form optically pure helically chiral cyclometalated platinum(II) or palladium(II) complexes circularly polarized light emitting materials. This induced reaction has stereospecificity, as shown in FIG. 3.
(2) Optical pure raw materials are economically and easily obtainable: The two chiral optically pure enantiomers for the preparation of central chiral L⁵ tetradentate ligands are commercially available and economically feasible compounds, which facilitates the large-scale preparation of two chiral optically pure tetradentate ligands.
(3) Chiral separation is unneeded for the preparation of the circularly polarized light emitting materials: The two chiral optically pure enantiomers of helically chiral tetradentate cyclometalated platinum(II) and palladium(II) complexes can be conveniently prepared through the two chiral optically pure tetradentate ligands mentioned above. Circularly polarized light emitting materials do not require chiral column separation and purification, greatly reducing the preparation cost of the materials.
(4) High chemical and thermal stabilities: The designed and developed tetradentate ligand can coordinate well with the Pt(II) and Pd(II) ions hybridized with dsp² to form stable and rigid quadrilateral configurations, making the complexes with high chemical stability; meanwhile, due to the significant steric hindrance effect between the designed central chiral ligand L⁵ and the other end ligand L¹, the entire metal complex molecule can form a stable helically chiral tetradentate cyclometalated complexes, which do not undergo racemization and lose its circularly polarized luminescence properties in solution or during high-temperature sublimation.
(5) Aromatic system chiral fragments: The central chiral fragment in the material molecules are benzocarbene and its derivatives, all of which are aromatic systems with high chemical stability; chiral fragments of benzocarbenes make a significant contribution to the charge distribution in the frontier orbitals and to the excitation of triplet states in material molecules. They can participate in the charge transfer process of excited states, which is beneficial for improving the photophysical properties of material molecules, such as quantum efficiency.

### BRIEF DESCRIPTION OF THE DRAWINGS

FIG. 1 is the propagation mode of sunlight (linear propagation);

FIG. 2 is the propagation mode of circularly polarized luminescent light;

FIG. 3 is the design concept diagram of optically pure spiral chiral tetradentated cyclometalated complexes with metal ions as the center for circularly polarized light emitting materials;

FIG. 4 is the distribution of electrons and holes in the lowest excited singlet state (T₁) of (S,R)—P—PtA1 and its enantiomer (R,S)-M-PtA1 obtained through experimental optimization using DFT theory, FIG. (A) is the molecular structure of (S,R)—P—PtA1, FIG. (B) is the distribution of electrons in T₁ of (S,R)—P—PtA1, FIG. (C) is the distribution of holes in T₁ of (S,R)—P—PtA1, FIG. (D) is the distribution of electrons in T₁ of (R,S)-M-PtA1, FIG. (E) is the distribution of holes in T₁ of (R,S)-M-PtA1; FIG. (F) is the molecular structure of (R,S)-M-PtA1;

FIG. 5 (A) is the high performance liquid chromatography (HPLC) spectrum of a mixture of (S,R)—P—PtA1 and its enantiomer (R,S)-M-PtA1 with a mass ratio of 1:1, (B) is the HPLC spectrum of optically pure (R,S)-M-PtA1, (C) is the HPLC spectrum of optically pure (S,R)—P—PtA1;

FIG. 6 (A) is the high performance liquid chromatography (HPLC) spectrum of a mixture of (R,R)—P—PtA and its enantiomer (S,S)-M-PtA with a mass ratio of 1:1, (B) is the HPLC spectrum of optically pure (R,R)—P—PtA3, (C) is the HPLC spectrum of optically pure (S,S)-M-PtA;

FIG. 7 (A) is the high performance liquid chromatography (HPLC) spectrum of a mixture of (R)-Bn-M-PtA8 and its enantiomer (S)-Bn-P—PtA8 with a mass ratio of 1:1, (B) is the HPLC spectrum of optically pure (R)-Bn-M-PtA8, (C) is the HPLC spectrum of optically pure (S)-Bn-P—PtA8;

FIG. 8 is the thermogravimetric analysis curve of (S,R)—P—PtA1;

FIG. 9 (A) is the circular dichroic spectrogram (CD) spectrum of a mixture of (S,R)—P—PtA1 and its enantiomer (R,S)-M-PtA1 with a mass ratio of 1:1 in dichloromethane solution, (B) is the CD spectrum of (S,R)—P—PtA2 and its enantiomer (R,S)-M-PtA2 in dichloromethane solution, (C) is the CD spectrum of (R,R)-M-PtA3 and its enantiomer (S,S)—P—PtA3 in dichloromethane solution, (D) is the CD spectrum of (R)-iPr-M-PtA4 and its enantiomer (S)-iPr—P—PtA4 in dichloromethane solution;

FIG. 10 (A) is the CD spectrum of (R)-iBu-M-PtA6 and its enantiomer (S)-iBu-P—PtA6 in dichloromethane solution, (B) is the CD spectrum of (R)-2MeiBu-M-PtA7 and its enantiomer (S)-2MeiBu-P—PtA7 in dichloromethane solution, (C) is the CD spectrum of (R)-Bn-M-PtA8 and its enantiomer (S)-Bn-P—PtA8 in dichloromethane solution, (D) is the CD spectrum of (R)-2MeBn-M-PtA9 and its enantiomer (S)-2MeBn-P—PtA9 in dichloromethane solution;

FIG. 11 (A) is the CD spectrum of (S,R)-2Ph-M-PtA11 and its enantiomer (R,S)-2Ph-P—PtA11 in dichloromethane solution, (B) is the CD spectrum of (R,R)-M-PtA12 and its enantiomer (S,S)—P—PtA12 in dichloromethane solution, (C) is the CD spectrum of (R,S)-M-PtB1 and its enantiomer (S,R)—P—PtB1 in dichloromethane solution, (D) is the CD spectrum of (R,S)-M-PtC1 and its enantiomer (S,R)—P—PtC1 in dichloromethane solution;

FIG. 12 (A) is the CD spectrum of (S)-2MeiPr—P—PtA5 in dichloromethane solution, (B) is the CD spectrum of (R,S)-M-PdA1 and its enantiomer (S,R)—P—PdA1 in dichloromethane solution, (C) is the CD spectrum of (S)-M-PtD1 and its enantiomer (R)—P—PtD1 in dichloromethane solution, (D) is the CD spectrum of (R,S)-M-PtC1 and its enantiomer (S,R)—P—PtC1 in dichloromethane solution;

FIG. 13 (A) is the circularly polarized luminescence (CPL) spectrum of (R,S)-M-PtA1 and its enantiomer (S,R)—P—PtA1 in dichloromethane solution, (B) is the CPL spectrum of (R,S)-M-PtA2 and its enantiomer (S,R)—P—PtA2 in dichloromethane solution, (C) is the dissymmetry factor (g_{PL}) curve of (R,S)-M-PtA1 and its enantiomer (S,R)—P—PtA1 at different wavelengths in dichloromethane solution, (D) is the dissymmetry factor (g_{PL}) curve of (R,S)-M-PtA2 and its enantiomer (S,R)—P—PtA2 at different wavelengths in dichloromethane solution;

FIG. 14 (A) is the circularly polarized luminescence (CPL) spectrum of (R,R)-M-PtA3 and its enantiomer (S,S)—P—PtA3 in dichloromethane solution, (B) is the dissymmetry factor (g_{PL}) curve of (R,R)-M-PtA3 and its enantiomer (S,S)—P—PtA3 at different wavelengths in dichloromethane solution;

FIG. 15 (A) is the circularly polarized luminescence (CPL) spectrum of (R)-iPr-M-PtA4 and its enantiomer (S)-iPr—P—PtA4 in dichloromethane solution, (B) is the CPL spectrum of (R)-iBu-M-PtA6 and its enantiomer (S)-iBu-P—PtA6 in dichloromethane solution, (C) is the dissymmetry factor (g_{PL}) curve of (R)-iPr-M-PtA4 and its enantiomer (S)-iPr—P—PtA4 at different wavelengths in dichloromethane solution, (D) is the dissymmetry factor (g_{PL}) curve of (R)-iBu-M-PtA6 and its enantiomer (S)-iBu-P—PtA6 at different wavelengths in dichloromethane solution;

FIG. 16 (A) is the circularly polarized luminescence (CPL) spectrum of (R)-Bn-M-PtA8 and its enantiomer (S)-Bn-P—PtA8 in dichloromethane solution, (B) is the dissymmetry factor (g_{PL}) curve of (R)-Bn-M-PtA8 and its enantiomer (S)-Bn-P—PtA8 at different wavelengths in dichloromethane solution;

FIG. 17 (A) is the circularly polarized luminescence (CPL) spectrum of (R)-2MeBn-M-PtA9 and its enantiomer (S)-2MeBn-P—PtA9 in dichloromethane solution, (B) is the CPL spectrum of (S,R)-2Ph-M-PtA11 and its enantiomer (R,S)-2Ph-P—PtA11 in dichloromethane solution, (C) is the dissymmetry factor (g_{PL}) curve of (R)-2MeBn-M-PtA9 and its enantiomer (S)-2MeBn-P—PtA9 at different wavelengths in dichloromethane solution, (D) is the dissymmetry factor (g_{PL}) curve of (S,R)-2Ph-M-PtA11 and its enantiomer (R,S)-2Ph-P—PtA11 at different wavelengths in dichloromethane solution;

FIG. 18 (A) is the circularly polarized luminescence (CPL) spectrum of (S)-M-PtD1 and its enantiomer (R)—P—PtD1 in dichloromethane solution, (B) is the dissymmetry factor (g_{PL}) curve of (S)-M-PtD1 and its enantiomer (R)—P—PtD1 at different wavelengths in dichloromethane solution;

FIG. 19 (A) is the emission spectrum of optically pure (R,S)-M-PtA1 and its enantiomer (S,R)—P—PtA1 in dichloromethane solution, (B) is the emission spectrum of optically pure (R,S)-M-PtA2 and its enantiomer (S,R)—P—PtA2 in dichloromethane solution, (C) is the emission spectrum of optically pure (R)-iPr-M-PtA4 and its enantiomer (S)-iPr—P—PtA4 in dichloromethane solution, (D) is the emission spectrum of optically pure (S)-2MeiPr—P—PtA5 in dichloromethane solution, (E) is the emission spectrum of optically pure (R)-iBu-M-PtA6 and its enantiomer (S)-iBu-P—PtA6 in dichloromethane solution, (F) is the emission spectrum of optically pure (R)-2MeiBu-M-PtA7 and its enantiomer (S)-2MeiBu-P—PtA7 in dichloromethane solution, (G) is the emission spectrum of optically pure (R)-Bn-M-PtA8 and its enantiomer (S)-Bn-P—PtA8 in dichloromethane solution, (H) is the emission spectrum of optically pure (R)-2MeBn-M-PtA9 and its enantiomer (S)-2MeBn-P—PtA9 in dichloromethane solution, (I) is the emission spectrum of optically pure (S,R)-2Ph-M-PtA11 and its enantiomer (R,S)-2Ph-P—PtA11 in dichloromethane solution, (J) is the emission spectrum of optically pure (R,R)-M-PtA12 and its enantiomer (S,S)—P—PtA12 in dichloromethane solution, (K) is the emission spectrum of optically pure (S)-M-PtD1 and its enantiomer (R)—P—PtD1 in dichloromethane solution, (L) is the emission spectrum of optically pure (R,S)-M-PtE1 and its enantiomer (S,R)—P—PtE1 in dichloromethane solution;

FIG. 20 is the structure layer diagram of organic light-emitting diode device of the present disclosure. Among them, 110 represents the substrate, 120 represents the anode, 130 represents the hole injection layer, 140 represents the hole transport layer, 150 represents the luminescent layer, 160 represents the hole blocking layer, 170 represents the electron transport layer, 180 represents the electron injection layer, and 190 represents the cathode.

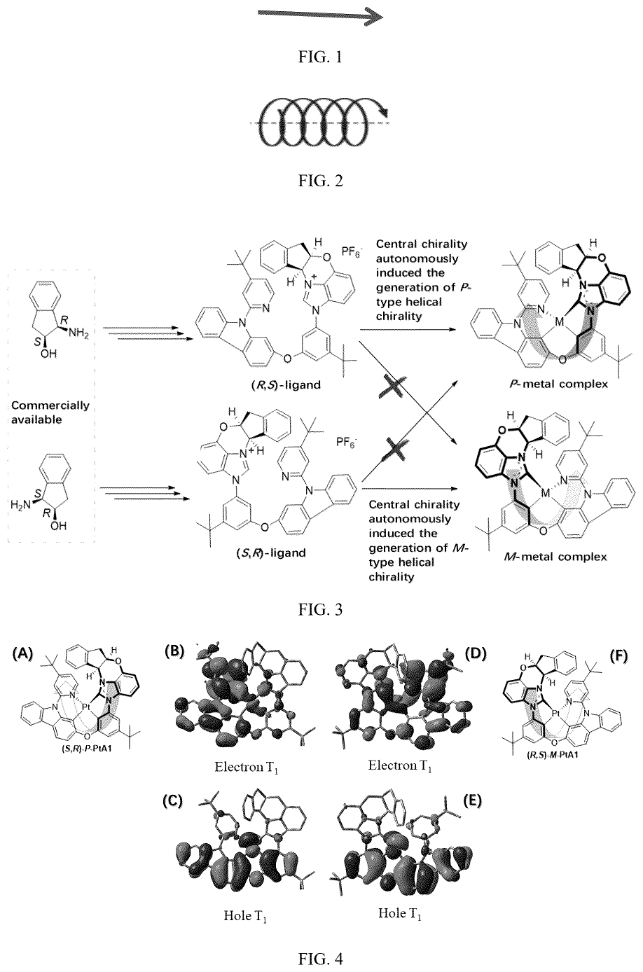

### DETAILED DESCRIPTION

Contents of the present disclosure are described in detail below. Following description of constituent elements described below is sometimes based on representative embodiments or specific examples of the present disclosure, but the present disclosure is not limited to such embodiments or specific examples.

The present disclosure may be more readily understood by reference to the following detailed description and examples included therein. Before compounds, devices, and/or methods of the present disclosure are disclosed and described, it should be understood that they are not limited to specific synthetic methods or specific reagents unless otherwise specified, as those may vary. It should also be understood that terminologies used in the present disclosure is for a purpose of describing particular aspects only but is not intended to be limiting. While any method and material similar or equivalent to those described herein may be used in this practice or test, example methods and materials are now described.

As used in the specification and appended claims, terms “a,” “an,” and “the” involves plural referents unless the context clearly dictates otherwise. Thus, for example, reference to a “component” includes a mixture of two or more components.

A term “optional” or “optionally” as used herein means that a subsequently described event or condition may or may not occur, and that such description includes both instances in which the described event or condition occurs and instances in which it does not.

Components useful in preparing compositions of the present disclosure are disclosed, as well as the compositions themselves to be used in the methods disclosed in the present disclosure. These and other materials are disclosed, and it is to be understood that combinations, subsets, interactions, groups, etc. of these materials are disclosed, and that while specific references to each of various individual and general combinations and permutations of these compounds are not specifically disclosed, each of them is specifically contemplated and described. For example, if a particular compound is disclosed and discussed and many modifications that can be made to many molecules comprising the compound are discussed, each combination and permutation of the compound and possible modifications are specifically contemplated unless specifically indicated to the contrary. Thus, if a class of molecules A, B and C are disclosed as well as a class of molecules D, E and F and an example of a combination molecule A-D, then even if each is not individually recited, it is contemplated that they are individually and collectively disclosed, namely, A-E, A-F, B-D, B-E, B-F, C-D, C-E and C-F. Likewise, any subset or combination of these are also disclosed. For example, subgroups such as A-E, B-F, and C-E are also disclosed. This concept applies to all aspects of the present disclosure, including, but not limited to, steps in methods of making and using the composition. Thus, if there are various additional steps that can be performed, it should be understood that each of these additional steps can each be performed in a particular implementation of the method or a combination of implementations thereof.

A linking atom used in the present disclosure is capable of linking two groups, for example, linking N and C. The linking atom can optionally (if valence bonds allow) be attached to other chemical groups. For example, an oxygen atom will not have any other chemical group attachment since once two atoms (e. g., N or C) are bonded, the valence bonds are already fully used. In contrast, when the linking atom is carbon, two additional chemical groups can be attached to the carbon atom. Suitable chemical groups include, but are not limited to, hydrogen, hydroxyl, alkyl, alkoxy, ═O, halogen, nitro, amine, amide, mercapto, aryl, heteroaryl, cycloalkyl, and heterocyclyl.

The term “cyclic structure” or similar terms used in the present disclosure refer to any cyclic chemical structure, including but not limited to aryl, heteroaryl, cycloalkyl, cycloene, heterocyclic, carbene, and N-heterocyclic carbene.

A term “substituted” or the like as used herein includes all permissible substituents of an organic compound. Broadly, permissible substituents include cyclic and acyclic, branched and unbranched, carbocyclic and heterocyclic, aromatic and non-aromatic substituents of organic compounds. For example, exemplary substituents include following content. For suitable organic compounds, the permissible substituents may be one or more, the same or different. For a purposes of the present disclosure, heteroatom (e. g., nitrogen) can have a hydrogen substituent and/or any permissible substituent of the organic compound of the present disclosure satisfying valence bonds of the heteroatom. The present disclosure is not intended to be limiting in any way with the permissible substituents of the organic compound. As such, a term “substituted” or “substituted with” encompasses an implicit condition that such a substitution conforms to permissible valence bonds of a substituted atom and the substituent, and that the substitution results in stable compounds (e. g., compounds that do not spontaneously undergo conversion (e. g., by rearrangement, cyclization, elimination, etc.)). In certain aspects, individual substituents can be further optionally substituted (i.e., further substituted or unsubstituted) unless explicitly stated to the contrary.

In defining various terms, “R¹”, “R²” and “R³” are used in the present disclosure as general symbols to represent various specific substituents. These symbols can be any substituent, not limited to those disclosed herein, which, when defined in an example as certain substituents, can also be defined in another example as some other substituents.

A term “alkyl” as used herein is a branched or unbranched saturated hydrocarbon group of 1 to 60 carbon atoms such as methyl, ethyl, n-propyl, isopropyl, n-butyl, iso-butyl, sec-butyl, tert-butyl, n-pentyl, iso-pentyl, sec-pentyl, neopentyl, hexyl, heptyl, octyl, nonyl, decyl, dodecyl, tetradecyl, hexadecyl, eicosyl, tetracosyl and the like. The alkyl may be cyclic or acyclic. The alkyl may be branched or unbranched. The alkyl may also be substituted or unsubstituted. For example, the alkyl may be substituted with one or more group including, but not limit to, optionally substituted alkyl, cycloalkyl, alkoxy, amino, ether, halogen, hydroxy, nitro, silyl, sulfo-oxo and thiol as described herein.

Throughout this specification, “alkyl” generally refer to both unsubstituted and substituted alkyl. However, substituted alkyl is also specifically mentioned in this present disclosure by determining specific substituents on the alkyl. For example, a term “halogenated alkyl” or “haloalkyl” specifically refers to an alkyl substituted with one or more halogens (e.g., fluorine, chlorine, bromine, or iodine). A term “alkylamino” specifically refers to an alkyl substituted with one or more amino, as described below or the like. When “alkyl” is used in one case and specific terms such as “alkyl alcohol” are used in another case, it is not intended to imply that the term “alkyl” does not simultaneously refer to specific terms such as “alkyl alcohol” or the like.

This practice is also applicable for other groups described herein. That is, when terms such as “cycloalkyl” refer to both unsubstituted and substituted cycloalkane moieties, the substituted moiety may additionally be specifically determined in the present disclosure. For example, a specifically substituted cycloalkyl may be referred to as, for example, “alkyl cycloalkyl”. Similarly, a substituted alkoxy may be specifically referred to as, for example, “haloalkoxy” and a specific substituted alkenyl may be, for example, “enol” or the like. Likewise, use of general terms such as “cycloalkyl” and specific terms such as “alkylcycloalkyl” does not imply that the general terms do not include the specific term at the same time.

The term “cycloalkyl” as used herein is a non-aromatic carbon-based ring of 3 to 30 carbon atoms consisting of at least three carbon atoms. Examples of cycloalkyl include, but are not limited to, cyclopropyl, cyclobutyl, cyclopentyl, cyclohexyl, cyclononyl, and the like. A term “heterocycloalkyl” is a type of cycloalkyl as defined above and is included in meaning of the term “cycloalkyl” in which at least one ring carbon atom is substituted by a heteroatom such as, but not limited to, a nitrogen, oxygen, sulfur or phosphorus atom. The cycloalkyl and heterocycloalkyl may be substituted or unsubstituted. The cycloalkyl and heterocycloalkyl may be substituted with one or more groups including, but not limited to, alkyl, cycloalkyl, alkoxy, amino, ether, halogen, hydroxy, nitro, silyl, sulfo-oxo and thiol as described herein.

Terms “alkoxy” and “alkoxy group” as used herein refer to an alkyl or cycloalkyl of 1 to 60 carbon atoms bonded by ether bonds. That is, “alkoxy” may be defined as —OR¹, where R1 is the alkyl or cycloalkyl as defined above. “Alkoxy” further includes alkoxy polymers just described. That is, the alkoxy may be a polyether such as —OR¹—OR² or —OR¹—(OR²)a-OR³, where “a” is an integer between 1 to 500, and R¹, R² and R³ are each independently an alkyl, a cycloalkyl, or a combination thereof.

A term “alkenyl” as used herein is a hydrocarbon of 2 to 60 carbon atoms with a structural formula containing at least one carbon-carbon double bond. An asymmetric structure such as (R¹R²)C═C(R³) contains E and Z isomers. This can be inferred in a structural formula of the present disclosure in which an asymmetric olefin is present, or it can be explicitly expressed by a bond symbol C═C. The alkenyl may be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halogen, hydroxy, ketone, azido, nitro, silyl, sulfo-oxo or thiol as described herein.

A term “cycloalkenyl” as used herein is a non-aromatic carbon-based ring of 3 to 60 carbon atoms which consists of at least 3 carbon atoms and contains at least one carbon-carbon double bond, i.e. C═C. Examples of cycloalkenyl include, but are not limited to, cyclopropenyl, cyclobutenyl, cyclopentenyl, cyclopentadienyl, cyclohexenoyl, cycloheptene, and the like. A term “heterocyclic alkenyl” is a type of cycloalkenyl as defined above and is included in meaning of the term “cycloalkenyl” in which at least one carbon atom of the ring is substituted with a heteroatom such as, but not limited to, a nitrogen, oxygen, sulfur or phosphorus atom. The cycloalkenyl and heterocyclic alkenyl may be substituted or unsubstituted. The cycloalkenyl and heterocyclic alkenyl may be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halogen, hydroxy, ketone, azido, nitro, silyl, sulfo-oxo or thiol as described herein.

A term “alkynyl” as used herein is a hydrocarbon having 2 to 60 carbon atoms and having a structural formula containing at least one carbon-carbon triple bond. The alkynyl may be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halogen, hydroxy, ketone, azido, nitro, silyl, sulfo-oxo or thiol as described herein.

A term “cycloalkynyl” as used herein is a non-aromatic carbon-based ring that contains at least 7 carbon atoms and contains at least one carbon-carbon triple bond. Examples of cycloalkynyl include, but are not limited to, cycloheptynyl, cyclooctynyl, cyclononynyl, and the like. A term “heterocyclic alkynyl” is a cycloalkenyl as defined above and is included within meaning of the term “cycloalkynyl” in which at least one of the carbon atoms of the ring is replaced by a heteroatom such as, but not limited to, a nitrogen, oxygen, sulfur or phosphorus atom. The cycloalkynyl and heterocyclic alkynyl may be substituted or unsubstituted. The cycloalkynyl and heterocyclic alkynyl may be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halogen, hydroxy, ketone, azido, nitro, silyl, sulfo-oxo or thiol as described herein.

A term “aryl” as used herein refers to a group containing 60 or less carbon atoms of any carbon-based aromatic group, including but not limited to benzene, naphthalene, phenyl, biphenyl, phenoxybenzene, and the like. The term “aryl” further includes “heteroaryl”, which is defined as a group containing an aromatic group having at least one heteroatom within a ring. Examples of heteroatoms include, but are not limited to, a nitrogen, oxygen, sulfur, or phosphorus atom. Likewise, a term “non-heteroaryl” (which is also included in the term “aryl”) defines an aromatic-containing group that is free of heteroatoms. The aryl may be substituted or unsubstituted. The aryl may be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halogen, hydroxy, ketone, azido, nitro, silyl, sulfo-oxo or thiol as described herein. A term “biaryl” is a particular type of aryl and is included in definition of “aryl”. The biaryl refers to two aryl joined together by a fused ring structure, as in naphthalene, or two aryl joined by one or more carbon-carbon bonds, as in biphenyl.

A term “aldehyde” as used herein is represented by a formula —C(O)H. Throughout this specification, “C(O)” is a short form of carbonyl (i.e., C═O).

A term “amine” or “amino” as used herein is represented by the formula —NR¹R², where R¹ and R² may independently be selected from hydrogen, alkyl, cycloalkyl, alkenyl, cycloalkenyl, alkynyl, cycloalkynyl, aryl or heteroaryl.

A term “alkylamino” as used herein is represented by a formula —NH(-alkyl), where the alkyl is as described in the present disclosure. Representative examples include, but are not limited to, methylamino, ethylamino, propylamino, iso-propylamino, butylamino, isobutylamino, sec-butylamino, tert-butylamino, pentylamino, isopentylamino, tert-pentylamino, hexylamino and the like.

A term “alkylamino” as used herein is represented by a formula —N(-alkyl)₂, where the alkyl is as described in the present disclosure. Representative example include, but are not limited to, dimethylamino, diethylamino, dipropylamino, diisopropylamino, dibutylamino, diisobutylamino, di-sec-butylamino, di-tert-butylamino, dipentylamino, diisopentylamino, di-tert-pentylamino, dihexylamino, N-ethyl-N-methylamino, N-methyl-N-propylamino, N-ethyl-N-propylamino and the like.

A term “carboxylic acid” as used herein is represented by a formula —C(O)OH.

A term “ester” as used herein is represented by a formula —OC(O)R¹ or —C(O)OR¹, where R¹ may be alkyl, cycloalkyl, alkenyl, cycloalkenyl, alkynyl, cycloalkynyl, aryl or heteroaryl as described herein. A term “polyester” as used herein is represented by a formula —(R¹O(O)C—R²—C(O)O)ₐ— or —(R¹O(O)C—R²—OC(O))ₐ—, where R¹ and R² can independently be alkyl, cycloalkyl, alkenyl, cycloalkenyl, alkynyl, cycloalkynyl, aryl or heteroaryl as described herein and “a” is an integer between 1 to 500. The term “polyester” is used to describe groups produced by reaction between a compound having at least two carboxyl and a compound having at least two hydroxy.

A term “ether” as used herein is represented by a formula R¹OR², where R¹ and R² may independently be alkyl, cycloalkyl, alkenyl, cycloalkenyl, alkynyl, cycloalkynyl, aryl or heteroaryl as described herein. A term “polyether” as used herein is represented by a formula —(R¹O—R²O)ₐ—, where R¹ and R² may independently be alkyl, cycloalkyl, alkenyl, cycloalkenyl, alkynyl, cycloalkynyl, aryl or heteroaryl as described herein and “a” is an integer between 1 to 500. Examples of polyether groups include polyethylene oxide, polypropylene oxide and polybutylene oxide.

A term “halogen” as used herein refers to halogen fluorine, chlorine, bromine and iodine.

A term “heterocyclyl” as used herein refers to monocyclic and polycyclic non-aromatic ring systems, and “heteroaryl” as used herein refers to monocyclic and polycyclic aromatic ring systems of not more than 60 carbon atoms, where at least one of ring members is not carbon. This term includes azetidinyl, dioxane, furyl, imidazolyl, isothiazolyl, isoxazolyl, morpholinyl, oxazolyl (oxazolyl including 1,2,3-oxadiazole, 1,2,5-oxadiazole and 1,3,4-oxadiazole), piperazinyl, piperidyl, pyrazinyl, pyrazolyl, pyridazinyl, pyridinyl, pyrimidinyl, pyrrolyl, pyrrolidinyl, tetrahydrofuran, tetrahydropyranyl, tetrazinyl including 1,2,4,5-tetrazinyl, tetrazolyl including 1,2,3,4-tetrazolyl and 1,2,4,5-tetrazolyl, thiadiazole including 1,2,3-thiadiazole, 1,2,5-thiadiazole and 1,3,4-thiadiazole, thiazolyl, thienyl, triazinyl including 1,3,5-triazinyl and 1,2,4-triazinyl, thiadiazolyl including 1,2,3-triazolinyl and 1,3,4-triazolinyl, and the like.

A term “hydroxyl” as used herein is represented by a formula —OH.

A term “ketone” as used herein is represented by a formula R¹C(O)R², where R¹ and R² may independently be alkyl, cycloalkyl, alkenyl, cycloalkenyl, alkynyl, cycloalkynyl, aryl or heteroaryl as described herein.

A term “azido” as used herein is represented by a formula —N₃.

A term “nitro” as used herein is represented by a formula —NO₂.

A term “nitrile” as used herein is represented by a formula —CN.

A term “silyl” as used herein is represented by a formula —SiR¹R²R³, where R¹, R² and R³ may independently be alkyl, cycloalkyl, alkoxy, alkenyl, cycloalkenyl, alkynyl, cycloalkynyl, aryl or heteroaryl as described herein.

A term “sulfo-oxo” as used herein is represented by a formula —S(O)R¹, —S(O)₂R¹, —OS(O)₂R¹ or —OS(O)₂OR¹, where R¹ may be alkyl, cycloalkyl, alkenyl, cycloalkenyl, alkynyl, cycloalkynyl, aryl or heteroaryl as described herein. Throughout this specification, “S(O)” is a short form of S═O. A term “sulfonyl” as used herein refers to a sulfo-oxo represented a formula —S(O)₂R¹, where R¹ may be alkyl, cycloalkyl, alkenyl, cycloalkenyl, alkynyl, cycloalkynyl, aryl or heteroaryl. A term “sulfone” as used herein is represented by a formula R¹S(O)₂R², where R¹ and R² may independently be alkyl, cycloalkyl, alkenyl, cycloalkenyl, alkynyl, cycloalkynyl, aryl or heteroaryl as described herein. A term “sulfoxide” as used herein is represented by a formula R¹S(O)R², where R¹ and R² may independently be alkyl, cycloalkyl, alkenyl, cycloalkenyl, alkynyl, cycloalkynyl, aryl or heteroaryl as described herein.

A term “thiol” as used herein is represented by a formula —SH.

“R¹”, “R²”, “R³” “Rⁿ” (where n is an integer) as used herein may independently have one or more of groups listed above. For example, if R¹ is linear alkyl, one hydrogen atom of the alkyl may be optionally substituted with hydroxy, alkoxy, alkyl, halogen or the like. Depending on a selected group, a first group may be incorporated within a second group, or the first group may be side linked (i.e., connected) to the second group. For example, for a phrase “an alkyl including amino”, the amino may be incorporated within a main chain of the alkyl. Alternatively, the amino may be linked to the main chain of the alkyl. Nature of the selected group may determine whether the first group is embedded in or linked to the second group.

The compound of that present disclosure may contain an “optionally substituted” moiety. In general, a term “substituted” (whether or not a term “optionally” exists before it) means that one or more hydrogen atoms of a given moiety are substituted with a suitable substituent. Unless otherwise stated, an “optionally substituted” group may have suitable substituents at each of substitutable positions of the group, and when more than one position in any given structure may be substituted with more than one substituent selected from groups designated, the substituents may be the same or different at each position. Combinations of substituents contemplated in the present disclosure are preferably combinations that form stable or chemically feasible compounds. It is also contemplated that in certain aspects, respective substituents may be further optionally substituted (i. e., further substituted or unsubstituted) unless explicitly stated to the contrary.

A structure of the compound may be represented by a following formula:

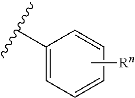

which is understood as equivalent to a following formula:

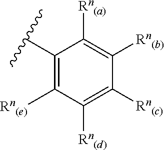

in which n is typically an integer. That is, Rⁿ is understood to indicate five individual substituents R^{n(a)}, R^{n(b)}, R^{n(c)}, R^{n(d)} and R^{n(e)}. “Individual substituent” means that each R substituent may be defined independently. For example, if R^{n(a)} is halogen in a case, R^{n(b)} is not necessarily halogen in this case.

R¹, R², R³, R⁴, R⁵, R⁶ etc. are mentioned several times in chemical structures and units disclosed and described in this disclosure. Any description of R¹, R², R³, R⁴, R⁵, R⁶ etc. in the specification is applicable to any structure or unit referring to R¹, R², R³, R⁴, R⁵, R⁶ etc. respectively, unless otherwise specified.

A term “fused ring” as used herein means that two adjacent substituents may be fused to form a six-member aromatic ring, a heteroaromatic ring, such as a benzene ring, a pyridine ring, a pyrazine ring, a pyridazine ring, a m-diazepine ring, and the like, as well as a saturated six or seven-member carbocyclic ring or carboheterocyclic ring, and the like.

This patent discloses compounds or complex complexes containing platinum. The term compound or complex is interchangeable in the present disclosure. In addition, the compounds disclosed in this disclosure have a neutral charg.

The compounds disclosed in this article are applicable to a variety of optical and electro-optical devices, including but not limited to light absorption devices, such as solar and photosensitive devices, organic light-emitting diodes (OLEDs), light emitting devices or devices that are compatible with light absorption and emission, as well as biomarkers for biological applications.

As mentioned above, the disclosed compounds are platinum or palladium complexes. Meanwhile, the compounds disclosed in this article can be used as the main materials for OLED applications, such as full-color displays.

The compounds disclosed in this article can be used for various applications. As a luminescent material, this compound can be used in organic light-emitting diodes (OLEDs), light-emitting devices and displays, as well as other light-emitting devices.

The compounds of the present disclosure can be prepared using various methods, including but not limited to those described in the embodiments provided herein.

In the present disclosure, organic optoelectronic devices can use methods such as spray coating, electron beam evaporation, vacuum evaporation, etc. to evaporate metal or conductive oxides and their alloys on a substrate to form an anode; On the surface of the prepared anode, a hole injection layer, a hole transport layer, a luminescent layer, an air barrier layer, and an electron transport layer are sequentially deposited, followed by a cathode deposition method. In addition to the above methods, organic electroluminescent devices are fabricated by sequentially evaporating the cathode, organic layer, and anode onto the substrate. The organic layer can also include multi-layer structures such as hole injection layer, hole transport layer, luminescent layer, hole blocking layer, and electron transport layer. In the present disclosure, the organic layer is prepared using polymer materials through solvent engineering (spin coating, tape casting, doctor plating, screen printing, inkjet printing or thermal imaging, etc.) instead of vapor deposition methods, which can reduce the number of device layers.

According to the present disclosure, the materials used in organic electroluminescent devices can be divided into top emission, low emission, or double-sided emission. The compound of the organic electroluminescent device according to the implementation scheme of the present disclosure can be applied to organic solar cells, lighting OLEDs, flexible OLEDs, organic photosensitive materials, organic thin film transistors and other electroluminescent devices, similar to the principle of organic light-emitting devices.

Unless otherwise specified, all commercial reagents involved in following tests are used directly after purchase and are not further purified. Both hydrogen and carbon nuclear magnetic resonance spectra were measured in deuterated chloroform (DCl₃) or deuterated dimethyl sulfoxide (DMSO-d₆), in which the hydrogen spectra is made using a 400 or 500 MHz nuclear magnetic resonance spectrometer and the carbon spectra is made using a 100 or 126 MHz nuclear magnetic resonance spectrometer, with chemical shifts being based on tetramethylsilane (TMS) or residual solvent. If CDCl₃ is used as a solvent, TMS (δ=0.00 ppm) and CDCl₃ (δ=77.00 ppm) are used as internal standards for the hydrogen and carbon spectra, respectively. If DMSO-d₆ is used as a solvent, TMS (δ=0.00 ppm) and DMSO-d₆ (δ=39.52 ppm) are used as internal standards for the hydrogen and carbon spectra, respectively. Following abbreviations (or combinations) are used to explain hydrogen peaks: s indicates a single peak, d indicates a double peak, t indicates a triple peak, q indicates a quadruple peak, p indicates a quintic peak, m indicates multiple peaks, and br indicates a wide peak. A high-resolution mass spectrum was measured on an ESI-QTOF mass spectrometer from Applied Biosystems, with an ionization mode of samples being electrospray ionization.

### Example 1

The synthetic route for (R,S)-M-PtA1 is as follows:

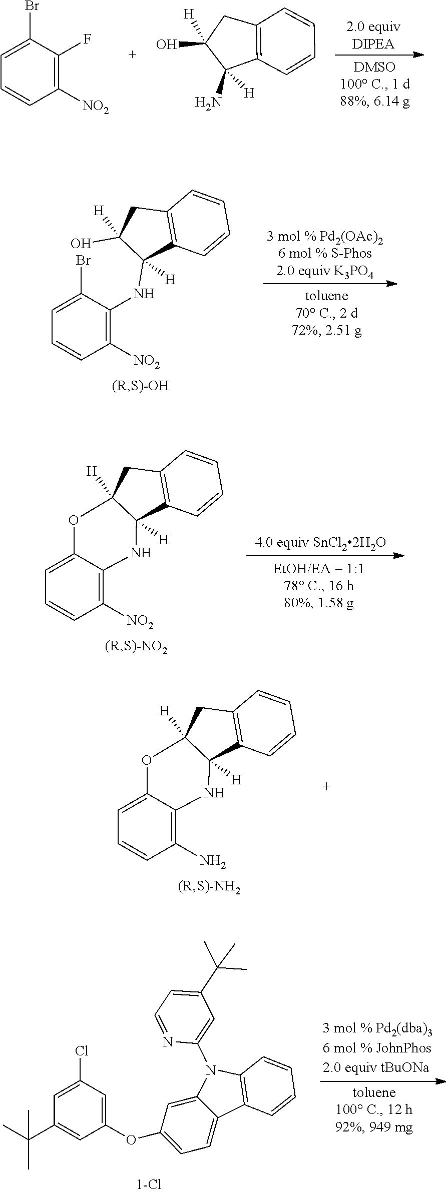 [SMILES: O=[N+]([O-])c1cccc(Br)c1F ; N[C@@H]1c2ccccc2C[C@@H]1O ; O=[N+]([O-])c1cccc(Br)c1N[C@@H]1c2ccccc2C[C@@H]1O ; O=[N+]([O-])c1cccc2c1N[C@@H]1c3ccccc3C[C@@H]1O2 ; Nc1cccc2c1N[C@@H]1c3ccccc3C[C@@H]1O2 ; CC(C)(C)c1cc(Cl)cc(Oc2ccc3c4ccccc4n(-c4cc(C(C)(C)C)ccn4)c3c2)c1]

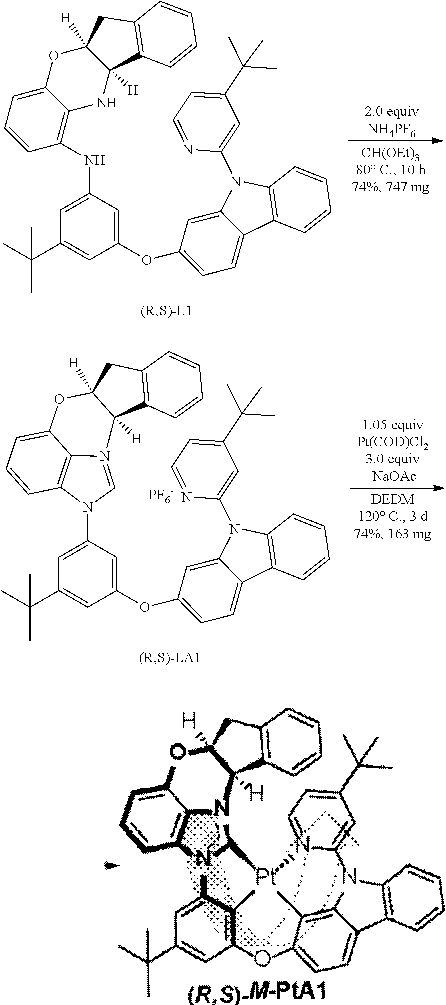

(1) Synthesis of (R,S)—OH: 1-bromo-2-fluoro-3-nitrobenzene (4.40 g, 20 mmol, 1.0 equiv) was added to a dry tube equipped with a magnetic stir bar. The tube was evacuated and backfilled with nitrogen, the evacuation and back fill procedure was repeated for another twice. Then DMSO (60 mL) and (1R,2S)-1-amino-2,3-dihydro-1H-inden-2-ol (2.98 g, 20 mmol, 1.0 equiv), N,N-Diisopropylethylamine (DIPEA) (5.17 g, 40 mmol, 2.0 equiv) were added under a nitrogen atmosphere. The tube was sealed and the mixture was stirred at a temperature of 100° C. for 1 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/dichloromethane (1:1) as eluent to obtain the desired product (R,S)—OH as a yellow solid 6.14 g in 88% yield. ¹H NMR (400 MHz, DMSO-d₆): δ(ppm) 2.77 (d, J=16.0 Hz, 1H), 3.01 (dd, J=16.4, 4.8 Hz, 1H), 4.29 (qd, J=4.8, 1.2 Hz, 1H), 5.15 (dd, J=10.0, 4.8 Hz, 1H), 5.39 (d, J=4.4 Hz, 1H), 6.86 (d, J=10.0 Hz, 1H), 6.95 (t, J=8.4 Hz, 1H), 7.18-7.26 (m, 3H), 7.32-7.35 (m, 1H), 7.96 (ddd, J=12.0, 8.4, 1.6 Hz, 2H). ¹³C NMR (100 MHz, DMSO-d₆): δ(ppm) 39.45, 63.82, 72.22, 114.93, 120.25, 124.40, 125.16, 125.86, 126.53, 127.73, 139.58, 140.72, 140.84, 141.95, 142.25.
(2) Synthesis of (R,S)—NO₂: Pd(OAc)₂ (58 mg, 0.26 mmol, 3 mol %), SPhos (214 mg, 0.52 mmol, 6 mol %) and K₃PO₄ (5.52 g, 26 mmol, 2.0 equiv) were added to a dry tube equipped with a magnetic stir bar. The tube was evacuated and backfilled with nitrogen, the evacuation and back fill procedure was repeated for another twice. Then (R,S)—OH (4.54 g, 13.00 mmol, 1.0 equiv) and toluene (52 mL) were added under a nitrogen atmosphere. The tube was sealed and the mixture was stirred at a temperature of 70° C. for 2 days, cooled down to ambient temperature. The solvent was removed under reduced pressure and the residue was purified through column chromatography on silica gel using petroleum ether/dichloromethane (10:1) as eluent to obtain the desired product (R,S)—NO₂ as a yellow solid 2.51 g in 72% yield. ¹H NMR (400 MHz, DMSO-d₆): δ(ppm) 3.09 (d, J=16.4 Hz, 1H), 3.29-3.34 (m, 1H), 4.64 (t, J=4.0 Hz, 1H), 5.16 (d, J=4.0 Hz, 1H), 6.53 (dd, J=8.8, 7.6 Hz, 1H), 6.98-7.00 (m, 1H), 7.19-7.25 (m, 2H), 7.30-7.32 (m, 1H), 7.35-7.38 (m, 1H), 7.66 (dd, J=8.8, 1.6 Hz, 1H), 9.19 (d, J=5.2 Hz, 1H). ¹³C NMR (100 MHz, DMSO-d₆): δ(ppm) 37.33, 56.94, 74.86, 114.31, 118.41, 121.24, 124.54, 125.33, 126.77, 127.73, 130.99, 132.88, 139.51, 143.23, 143.96.
(3) Synthesis of (R,S)—NH₂: (R,S)—NO₂ (2.23 g, 8.3 mmol, 1.0 equiv), SnCl₂·2·H₂O (7.49 g, 33.2 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (1:1) were added under a nitrogen atmosphere. The flask was sealed and the mixture was stirred at a temperature of 78° C. for 16 hours, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/dichloromethane (5:1:1) as eluent to obtain the desired product (R,S)—NH₂ as a pale yellow solid 1.58 g in 80% yield. ¹H NMR (400 MHz, DMSO-d₆): δ(ppm) 2.99 (d, J=16.0 Hz, 1H), 3.18 (dd, J=16.4, 4.0 Hz, 1H), 4.44 (t, J=3.6 Hz, 1H), 4.61 (s, 2H), 4.84-4.86 (m, 1H), 5.49 (d, J=5.6 Hz, 1H), 5.95 (dd, J=8.0, 1.2 Hz, 1H), 6.15 (dd, J=7.6, 1.2 Hz, 1H), 6.25 (t, J=8.0 Hz, 1H), 7.17-7.21 (m, 2H), 7.25-7.33 (m, 2H). ¹³C NMR (100 MHz, DMSO-d₆): δ(ppm) 37.56, 57.64, 74.81, 105.66, 107.45, 117.03, 119.30, 123.93, 125.31, 126.50, 127.28, 136.16, 139.99, 142.94, 144.84.
(4) Synthesis of (R,S)-L1: (R,S)—NH₂ (357 mg, 1.5 mmol, 1.0 equiv), 1-Cl (725 mg, 1.5 mmol, 1.0 equiv), Pd₂(dba)₃ (41 mg, 0.05 mmol, 3 mol %), JohnPhos (27 mg, 0.09 mmol, 6 mol %) and tBuONa (288 mg, 2.0 mmol, 3.0 equiv) were added to a dry tube equipped with a magnetic stir bar. The tube was evacuated and backfilled with nitrogen, the evacuation and back fill procedure was repeated for another twice. Then toluene (10 mL) was added under a nitrogen atmosphere. The tube was sealed and the mixture was stirred at a temperature of 100° C. for 12 hours, cooled down to ambient temperature. The solvent was removed under reduced pressure and the residue was purified through column chromatography on silica gel using petroleum ether/ethyl acetate (30:1) as eluent to obtain the desired product (R,S)-L1 as a light green, foamy solid 949 mg in 92% yield. ¹H NMR (400 MHz, DMSO-d₆): δ(ppm) 1.22 (s, 9H), 1.29 (s, 9H), 2.95 (d, J=16.0 Hz, 1H), 3.12 (dd, J=16.4, 4.0 Hz, 1H), 4.30 (t, J=3.6 Hz, 1H), 4.81 (t, J=4.4 Hz, 1H), 5.85 (d, J=5.6 Hz, 1H), 6.14 (t, J=2.0 Hz, 1H), 6.28 (t, J=7.6 Hz, 1H), 6.33 (dd, J=7.6, 1.2 Hz, 1H), 6.49 (t, J=2.0 Hz, 1H), 6.56 (dd, J=7.6, 1.6 Hz, 1H), 6.69 (t, J=1.6 Hz, 1H), 7.04 (dd, J=8.4, 2.0 Hz, 1H), 7.08 (d, J=7.6 Hz, 1H), 7.13 (t, J=7.2 Hz, 1H), 7.23 (d, J=7.6 Hz, 1H), 7.28-7.34 (m, 4H), 7.40-7.44 (m, 1H), 7.46 (dd, J=5.2, 1.6 Hz, 1H), 7.59 (d, J=0.8 Hz, 1H), 7.71 (d, J=8.0 Hz, 1H), 8.20 (t, J=8.4 Hz, 2H), 8.56 (d, J=5.6 Hz, 1H).
(5) Synthesis of (R,S)-LA1: (R,S)-L1 (822 mg, 1.2 mmol, 1.0 equiv), NH₄PF₆ (391 mg, 2.4 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (8 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using dichloromethane as eluent to obtain the desired product (R,S)-LA1 as a light green, foamy solid 747 mg in 74% yield. ¹H NMR (500 MHz, DMSO-d₆): δ(ppm) 1.30 (s, 9H), 1.37 (s, 9H), 3.30 (dd, J=16.5, 2.5 Hz, 1H), 3.55 (dd, J=16.5, 4.5 Hz, 1H), 5.41-5.43 (m, 1H), 6.35 (d, J=4.0 Hz, 1H), 7.17 (d, J=8.0 Hz, 1H), 7.21 (dd, J=8.5, 2.0 Hz, 1H), 7.30-7.36 (m, 4H), 7.39 (t, J=7.5 Hz, 1H), 7.43-7.49 (m, 3H), 7.50 (t, J=2.0 Hz, 1H), 7.50-7.56 (m, 2H), 7.60 (d, J=7.5 Hz, 1H), 7.64 (t, J=1.5 Hz, 1H), 7.68 (d, J=1.0 Hz, 1H), 7.74 (d, J=8.0 Hz, 1H), 8.24 (d, J=7.5 Hz, 1H), 8.33 (d, J=8.5 Hz, 1H), 8.58 (d, J=5.5 Hz, 1H), 10.46 (s, 1H).
(6) Synthesis of (R,S)-M-PtA1: (R,S)-LA1 (101 mg, 0.12 mmol, 1.0 equiv), Pt(COD)Cl₂ (47 mg, 0.13 mmol, 1.05 equiv) and NaOAc (30 mg, 0.36 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (7 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (R,S)-M-PtA1 as a pale yellow solid 163 mg in 74% yield. (Note: COD denotes 1,5-cyclooctadiene) ¹H NMR (500 MHz, DMSO-d₆): δ(ppm) 1.06 (s, 9H), 1.48 (s, 9H), 3.32 (d, J=17.0 Hz, 1H), 3.43 (dd, J=17.0, 4.0 Hz, 1H), 5.21 (t, J=3.0 Hz, 1H), 6.27 (d, J=3.0 Hz, 1H), 6.83-6.87 (m, 2H), 6.97 (d, J=3.5 Hz, 1H), 7.10-7.13 (m, 2H), 7.31-7.40 (m, 5H), 7.44 (d, J=1.0 Hz, 1H), 7.56 (d, J=6.5 Hz, 2H), 7.84 (d, J=8.0 Hz, 1H), 7.84 (d, J=1.5 Hz, 1H), 7.94 (d, J=8.0 Hz, 1H), 8.15 (dd, J=8.0, 1.5 Hz, 1H), 9.92 (d, J=6.5 Hz, 1H).

### Example 2

The synthetic route for (S,R)—P—PtA1 is as follows:

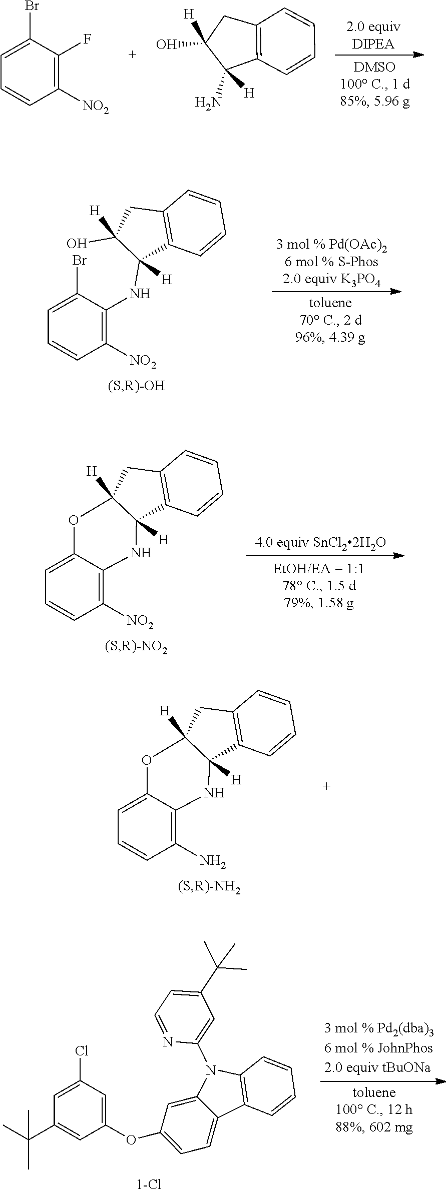 [SMILES: O=[N+]([O-])c1cccc(Br)c1F ; N[C@H]1c2ccccc2C[C@H]1O ; O=[N+]([O-])c1cccc(Br)c1N[C@H]1c2ccccc2C[C@H]1O ; O=[N+]([O-])c1cccc2c1N[C@H]1c3ccccc3C[C@H]1O2 ; Nc1cccc2c1N[C@H]1c3ccccc3C[C@H]1O2 ; CC(C)(C)c1cc(Cl)cc(Oc2ccc3c4ccccc4n(-c4cc(C(C)(C)C)ccn4)c3c2)c1]

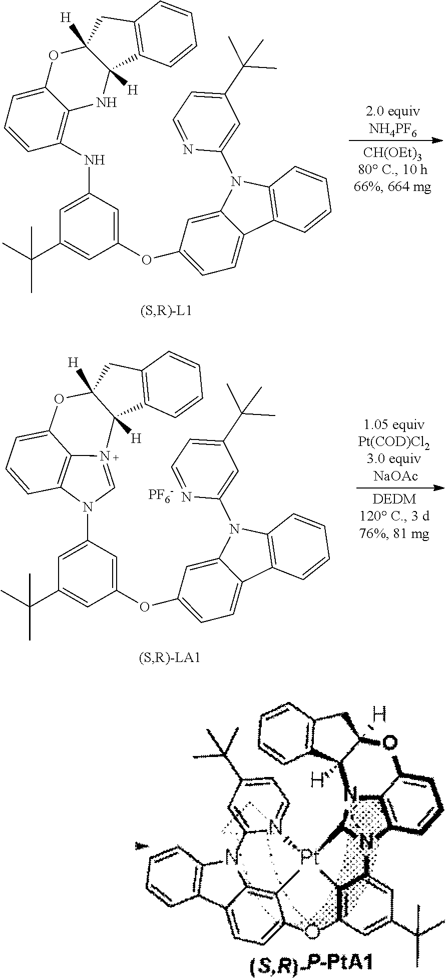

(1) Synthesis of (S,R)—OH: 1-bromo-2-fluoro-3-nitrobenzene (4.40 g, 20 mmol, 1.0 equiv) was added to a dry tube equipped with a magnetic stir bar. The tube was evacuated and backfilled with nitrogen, the evacuation and back fill procedure was repeated for another twice. Then DMSO (60 mL) and (1S,2R)-1-amino-2,3-dihydro-1H-inden-2-ol (2.98 g, 20 mmol, 1.0 equiv), N,N-Diisopropylethylamine (DIPEA) (5.17 g, 40 mmol, 2.0 equiv) were added under a nitrogen atmosphere. The tube was sealed and the mixture was stirred at a temperature of 100° C. for 1 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/dichloromethane (1:1) as eluent to obtain the desired product (S,R)—OH as a yellow solid 5.96 g in 85% yield. ¹H NMR (400 MHz, DMSO-d₆): δ(ppm) 2.78 (d, J=16.4 Hz, 1H), 3.01 (dd, J=16.0, 4.4 Hz, 1H), 4.29 (q, J=4.0 Hz, 1H), 5.15 (dd, J=10.0, 4.8 Hz, 1H), 5.39 (d, J=4.0 Hz, 1H), 6.86 (d, J=10.0 Hz, 1H), 6.95 (t, J=8.0 Hz, 1H), 7.18-7.26 (m, 3H), 7.32-7.35 (m, 1H), 7.96 (ddd, J=12.0, 8.0, 1.2 Hz, 2H). ¹³C NMR (100 MHz, DMSO-d₆): δ(ppm) 39.44, 63.81, 72.22, 114.92, 120.24, 124.40, 125.15, 125.85, 126.52, 127.72, 139.57, 140.71, 140.83, 141.95, 142.25.
(2) Synthesis of (S,R)—NO₂: Pd(OAc)₂ (115 mg, 0.51 mmol, 3 mol %), SPhos (419 mg, 1.02 mmol, 6 mol %) and K₃PO₄ (7.22 g, 34 mmol, 2.0 equiv) were added to a dry tube equipped with a magnetic stir bar. The tube was evacuated and backfilled with nitrogen, the evacuation and back fill procedure was repeated for another twice. Then (S,R)—OH (5.94 g, 17.00 mmol, 1.0 equiv) and toluene (68 mL) were added under a nitrogen atmosphere. The tube was sealed and the mixture was stirred at a temperature of 70° C. for 2 days, cooled down to ambient temperature. The solvent was removed under reduced pressure and the residue was purified through column chromatography on silica gel using petroleum ether/dichloromethane (10:1) as eluent to obtain the desired product (S,R)—NO₂ as a orange yellow solid 4.39 g in 96% yield. ¹H NMR (400 MHz, DMSO-d₆): δ(ppm) 3.09 (d, J=16.4 Hz, 1H), 3.30-3.34 (m, 1H), 4.64-4.66 (m, 1H), 5.15-5.17 (m, 1H), 6.51-6.55 (m, 1H), 6.98 (d, J=7.6 Hz, 1H), 7.19-7.25 (m, 2H), 7.30-7.31 (m, 1H), 7.36-7.38 (m, 1H), 7.66 (dt, J=8.8, 1.6 Hz, 1H), 9.17 (d, J=4.8 Hz, 1H). ¹³C NMR (100 MHz, DMSO-d₆): δ(ppm) 37.31, 56.92, 74.85, 114.29, 118.38, 121.21, 124.51, 125.30, 126.75, 127.71, 131.00, 132.85, 139.48, 143.19, 143.94.
(3) Synthesis of (S,R)—NH₂: (S,R)—NO₂ (4.29 g, 16 mmol, 1.0 equiv), SnCl₂·2H₂O (14.44 g, 64.0 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (1:1) were added under a nitrogen atmosphere. The flask was sealed and the mixture was stirred at a temperature of 78° C. for 1.5 days, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/dichloromethane (5:1:1) as eluent to obtain the desired product (S,R)—NH₂ as a pale yellow solid 3.01 g in 79% yield. ¹H NMR (400 MHz, DMSO-d₆): δ(ppm) 2.99 (d, J=16.4 Hz, 1H), 3.19 (dd, J=16.0, 3.6 Hz, 1H), 4.44 (t, J=3.6 Hz, 1H), 4.60 (s, 2H), 4.85 (t, J=4.4 Hz, 1H), 5.48 (d, J=6.0 Hz, 1H), 5.95 (d, J=7.6 Hz, 1H), 6.15-6.16 (m, 1H), 6.25 (t, J=7.6 Hz, 1H), 7.16-7.21 (m, 2H), 7.25-7.32 (m, 2H). ¹³C NMR (100 MHz, DMSO-d₆): δ(ppm) 37.52, 57.60, 74.79, 105.60, 107.39, 116.98, 119.26, 123.89, 125.27, 126.47, 127.24, 136.13, 139.97, 142.90, 144.81.
(4) Synthesis of (S,R)-L1: (S,R)—NH₂ (238 mg, 1.0 mmol, 1.0 equiv), 1-Cl (483 mg, 1.0 mmol, 1.0 equiv), Pd₂(dba)₃ (28 mg, 0.03 mmol, 3 mol %), JohnPhos (18 mg, 0.06 mmol, 6 mol %) and tBuONa (192 mg, 2.0 mmol, 2.0 equiv) were added to a dry tube equipped with a magnetic stir bar. The tube was evacuated and backfilled with nitrogen, the evacuation and back fill procedure was repeated for another twice. Then toluene (7 mL) were added under a nitrogen atmosphere. The tube was sealed and the mixture was stirred at a temperature of 100° C. for 12 hours, cooled down to ambient temperature. The solvent was removed under reduced pressure and the residue was purified through column chromatography on silica gel using petroleum ether/ethyl acetate (30:1) as eluent to obtain the desired product (S,R)-L1 as a light green, foamy solid 602 mg in 88% yield. ¹H NMR (400 MHz, DMSO-d₆): δ(ppm) 1.22 (s, 9H), 1.29 (s, 9H), 2.95 (d, J=16.4 Hz, 1H), 3.12 (dd, J=16.4, 4.0 Hz, 1H), 4.30 (t, J=3.6 Hz, 1H), 4.82 (t, J=4.4 Hz, 1H), 5.86 (d, J=6.0 Hz, 1H), 6.14 (t, J=2.0 Hz, 1H), 6.28 (t, J=7.6 Hz, 1H), 6.34 (dd, J=8.0, 1.2 Hz, 1H), 6.50 (t, J=1.6 Hz, 1H), 6.57 (dd, J=7.6, 1.2 Hz, 1H), 6.69 (t, J=1.6 Hz, 1H), 7.05 (dd, J=8.4, 2.0 Hz, 1H), 7.08 (d, J=7.2 Hz, 1H), 7.14 (t, J=7.2 Hz, 1H), 7.23 (d, J=7.2 Hz, 1H), 7.29-7.35 (m, 4H), 7.40-7.44 (m, 1H), 7.46 (dd, J=5.6, 1.6 Hz, 1H), 7.59 (d, J=1.2 Hz, 1H), 7.72 (d, J=8.4 Hz, 1H), 8.20 (t, J=8.4 Hz, 2H), 8.56 (d, J=5.2 Hz, 1H).
(5) Synthesis of (S,R)-LA1: (S,R)-L1 (822 mg, 1.2 mmol, 1.0 equiv), NH₄PF₆ (391 mg, 2.4 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (8 mL) was added under a nitrogen atmosphere. The tube was sealed and the mixture was stirred at a temperature of 80° C. for 11 hours, cooled down to ambient temperature. The solvent was removed under reduced pressure and the residue was purified through column chromatography on silica gel using dichloromethane as eluent to obtain the desired product (S,R)-LA1 as a light green, foamy solid 664 mg in 66% yield. ¹H NMR (400 MHz, DMSO-d₆): δ(ppm) 1.38 (s, 9H), 1.38 (s, 9H), 3.25 (dd, J=16.4, 5.2 Hz, 1H), 3.44 (dd, J=16.4, 5.6 Hz, 1H), 5.44 (q, J=5.2 Hz, 1H), 6.32 (d, J=4.4 Hz, 1H), 6.99 (t, J=2.0 Hz, 1H), 7.04 (d, J=8.0 Hz, 1H), 7.09 (dd, J=8.4, 2.0 Hz, 1H), 7.24 (d, J=8.4 Hz, 1H), 7.29-7.37 (m, 5H), 7.38 (t, J=2.0 Hz, 1H), 7.41-7.46 (m, 2H), 7.57-7.58 (m, 2H), 7.61 (d, J=2.4 Hz, 1H), 7.66-7.69 (m, 2H), 8.08 (d, J=7.6 Hz, 1H), 8.11 (d, J=8.4 Hz, 1H), 8.56 (d, J=5.2 Hz, 1H), 9.31 (s, 1H).
(6) Synthesis of (S,R)—P—PtA1: (S,R)-LA1 (101 mg, 0.12 mmol, 1.0 equiv), Pt(COD)Cl₂ (47 mg, 0.13 mmol, 1.05 equiv) and NaOAc (30 mg, 0.36 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (7 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (S,R)—P—PtA1 as a pale yellow solid 81 mg in 76% yield. ¹H NMR (400 MHz, DMSO-d₆): δ(ppm) 1.19 (s, 9H), 1.47 (s, 9H), 3.31 (d, J=5.2 Hz, 1H), 3.48 (dd, J=17.2, 3.6 Hz, 1H), 5.41 (s, 1H), 6.34 (d, J=3.2 Hz, 1H), 6.86-6.89 (m, 2H), 7.07 (d, J=1.2 Hz, 1H), 7.11-7.16 (m, 2H), 7.32-7.43 (m, 5H), 7.53 (s, 1H), 7.59 (d, J=7.6 Hz, 1H), 7.69 (d, J=8.4 Hz, 1H), 7.89 (d, J=8.0 Hz, 1H), 7.94 (d, J=8.4 Hz, 1H), 7.96 (d, J=1.2 Hz, 1H), 8.16-8.18 (m, 1H), 9.86 (d, J=6.4 Hz, 1H).

### Example 3

The synthetic route for (R,S)-M-PdA1 is as follows:

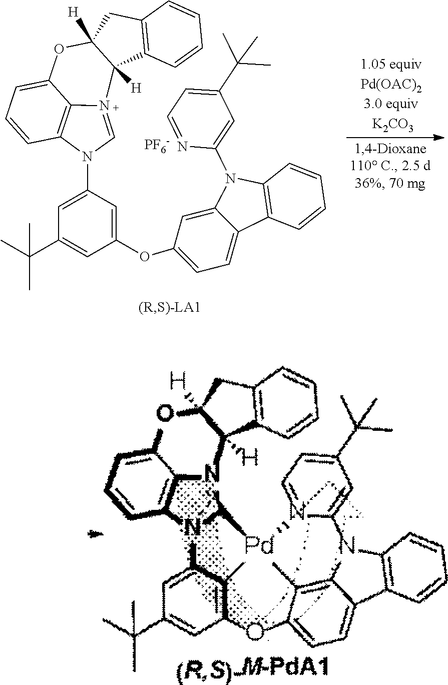

(1) Synthesis of (R,S)-M-PdA1: (R,S)-LA1 (200 mg, 0.24 mmol, 1.0 equiv), Pd(OAc)₂ (56 mg, 025 mmol, 1.05 equiv) and K₂CO₃ (99 mg, 0.72 mmol, 3.0 equiv) were added to a dry tube equipped with a magnetic stir bar. The tube was evacuated and backfilled with nitrogen, the evacuation and back fill procedure was repeated for another twice. Subsequently, 1,4-Dioxane (14 mL) were added under a nitrogen atmosphere, and the reaction mixture was bubbled with nitrogen for 30 minutes. The tube was sealed and the mixture was stirred at a temperature of 120° C. for 2.5 days, cooled down to ambient temperature. The solvent was removed under reduced pressure and the residue was purified through column chromatography on silica gel using petroleum ether/dichloromethane (2:1) as eluent to obtain the desired product (R,S)-M-PdA1 as a white solid 70 mg in 36% yield. ¹H NMR (500 MHz, DMSO-d₆): δ(ppm) 1.1 (s, 9H), 1.47 (s, 9H), 3.32-3.37 (m, 1H), 3.51 (dd, J=17.5, 4.0 Hz, 1H), 5.43 (t, J=4.0 Hz, 1H), 6.29 (d, J=3.5 Hz, 1H), 6.89 (d, J=8.0 Hz, 1H), 6.92 (t, J=8.0 Hz, 1H), 7.10 (d, J=1.5 Hz, 1H), 7.17 (t, J=7.5 Hz, 2H), 7.26 (d, J=8.0 Hz, 1H), 7.34-7.37 (m, 2H), 7.38-7.43 (m, 2H), 7.49 (d, J=7.5 Hz, 1H), 7.57 (d, J=1.5 Hz, 1H), 7.71 (d, J=8.5 Hz, 1H), 7.87-7.88 (m, 2H), 7.94 (d, J=8.0 Hz, 1H), 8.15 (dd, J=8.0, 1.0 Hz, 1H), 9.53 (d, J=6.0 Hz, 1H).

### Example 4

The synthetic route for (S,R)—P—PdA1 is as follows:

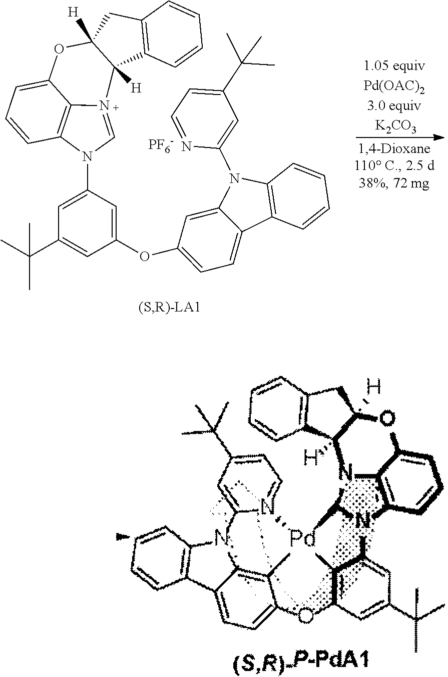

(1) Synthesis of (S,R)—P—PdA1: (S,R)-LA1 (200 mg, 0.24 mmol, 1.0 equiv), Pd(OAc)₂ (56 mg, 025 mmol, 1.05 equiv) and K₂CO₃ (99 mg, 0.72 mmol, 3.0 equiv) were added to a dry tube equipped with a magnetic stir bar. The tube was evacuated and backfilled with nitrogen, the evacuation and back fill procedure was repeated for another twice. Subsequently, 1,4-Dioxane (14 mL) were added under a nitrogen atmosphere, and the reaction mixture was bubbled with nitrogen for 30 minutes. The tube was sealed and the mixture was stirred at a temperature of 120° C. for 2.5 days, cooled down to ambient temperature. The solvent was removed under reduced pressure and the residue was purified through column chromatography on silica gel using petroleum ether/dichloromethane (2:1) as eluent to obtain the desired product (S,R)—P—PdA1 as a white solid 72 mg in 38% yield. ¹H NMR (500 MHz, DMSO-d₆): δ(ppm) 1.23 (s, 9H), 1.47 (s, 9H), 3.31-3.33 (m, 1H), 3.52 (dd, J=16.0, 3.5 Hz, 1H), 5.48 (t, J=4.0 Hz, 1H), 6.31 (d, J=3.5 Hz, 1H), 6.89 (d, J=8.0 Hz, 1H), 6.94 (t, J=7.0 Hz, 1H), 7.09 (d, J=1.5 Hz, 1H), 7.16-7.21 (m, 2H), 7.26 (d, J=8.5 Hz, 1H), 7.35-7.44 (m, 4H), 7.50 (d, J=7.5 Hz, 1H), 7.59 (d, J=1.5 Hz, 1H), 7.74 (d, J=8.0 Hz, 1H), 7.89 (d, J=8.0 Hz, 1H), 7.91 (d, J=2.0 Hz, 1H), 7.94 (d, J=8.5 Hz, 1H), 8.15 (dd, J=8.0, 1.0 Hz, 1H), 9.52 (d, J=6.0 Hz, 1H).

### Example 5

The synthetic route for (R,S)-M-PtA2 is as follows:

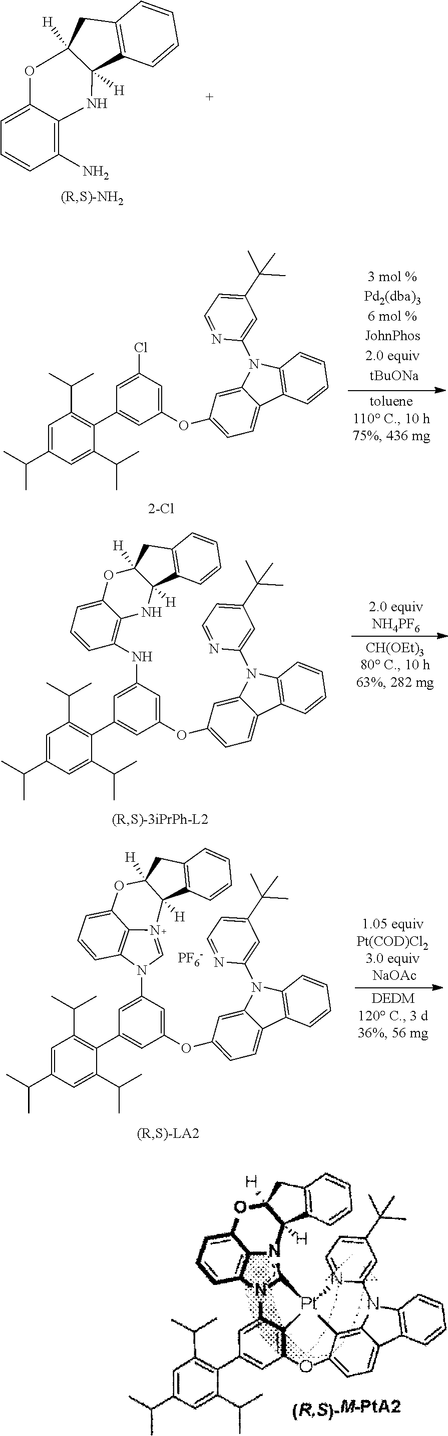

(1) Synthesis of (R,S)-3iPrPh-L2: (R,S)—NH₂ (167 mg, 0.70 mmol, 1.0 equiv), 2-Cl (441 mg, 0.70 mmol, 1.0 equiv), Pd₂(dba)₃ (19 mg, 0.021 mmol, 3 mol %), JohnPhos (13 mg, 0.042 mmol, 6 mol %) and tBuONa (135 mg, 1.4 mmol, 2.0 equiv) were added to a dry tube equipped with a magnetic stir bar. The tube was evacuated and backfilled with nitrogen, the evacuation and back fill procedure was repeated for another twice. Then toluene (6 mL) were added under a nitrogen atmosphere. The tube was sealed and the mixture was stirred at a temperature of 110° C. for 10 hours, cooled down to ambient temperature. The solvent was removed under reduced pressure and the residue was purified through column chromatography on silica gel using petroleum ether/ethyl acetate (30:1) as eluent to obtain the desired product (R,S)-3iPrPh-L2 as a yellow-green foamy solid 436 mg in 75% yield.
(2) Synthesis of (R,S)-LA2: (R,S)-3iPrPh-L2 (374 mg, 0.45 mmol, 1.0 equiv), NH₄PF₆ (147 mg, 0.90 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (4 mL) were added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using dichloromethane as eluent to obtain the desired product (R,S)-LA2 as a yellow foamy solid 282 mg in 63% yield. ¹H NMR (500 MHz, DMSO-d₆): δ(ppm) 0.93 (d, J=6.5 Hz, 6H), 1.20 (d, J=6.5 Hz, 6H), 1.31 (s, 9H), 2.54-2.60 (m, 2H), 2.84-2.92 (m, 1H), 3.31-3.34 (m, 1H), 3.58 (dd, J=16.5, 4.5 Hz, 1H), 5.43-5.46 (m, 1H), 6.40 (d, J=4.0 Hz, 1H), 7.04-7.05 (m, 1H), 7.07 (s, 2H), 7.20 (d, J=8.0 Hz, 1H), 7.24 (dd, J=8.5, 2.0 Hz, 1H), 7.31-7.35 (m, 2H), 7.39-7.42 (m, 2H), 7.45 (d, J=8.5 Hz, 2H), 7.48-7.50 (m, 2H), 7.53 (t, J=1.5 Hz, 1H), 7.59 (t, J=8.5 Hz, 1H), 7.66 (d, J=7.5 Hz, 1H), 7.69 (d, J=2.0 Hz, 1H), 7.70 (d, J=1.5 Hz, 1H), 7.73 (d, J=8.5 Hz, 1H), 8.24 (d, J=8.0 Hz, 1H), 8.34 (d, J=8.5 Hz, 1H), 8.56 (d, J=5.0 Hz, 1H), 10.49 (s, 1H).
(3) Synthesis of (R,S)-M-PtLA2: (R,S)-LA2 (150 mg, 0.15 mmol, 1.0 equiv), Pt(COD)Cl₂ (60 mg, 0.16 mmol, 1.05 equiv) and NaOAc (37 mg, 0.45 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (9 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (R,S)-M-PtLA2 as a pale yellow solid 56 mg in 36% yield. ¹H NMR (500 MHz, DMSO-d₆): δ(ppm) 1.09-1.59 (m, 12H), 1.27 (s, 9H), 1.29 (d, J=7.0 Hz, 6H), 2.80-2.90 (m, 2H), 2.93-2.98 (m, 1H), 3.29-3.36 (m, 1H), 3.50 (dd, J=16.5, 4.5 Hz, 1H), 5.51 (s, 1H), 6.40 (d, J=3.0 Hz, 1H), 6.77 (d, J=1.0 Hz, 1H), 6.83 (d, J=8.0 Hz, 1H), 6.90 (t, J=7.0 Hz, 1H), 7.13 (d, J=2.5 Hz, 2H), 7.17 (t, J=8.5 Hz, 1H), 7.24 (t, J=8.5 Hz, 2H), 7.33 (d, J=8.0 Hz, 1H), 7.35-7.38 (m, 2H), 7.39 (t, J=7.5 Hz, 1H), 7.43-7.46 (m, 1H), 7.66 (dd, J=7.5, 2.5 Hz, 2H), 7.92 (d, J=8.5 Hz, 1H), 7.95 (d, J=8.5 Hz, 1H), 8.04 (d, J=1.5 Hz, 1H), 8.18 (d, J=7.5 Hz, 1H), 9.84 (d, J=6.5 Hz, 1H).

### Example 6

The synthetic route for (S,R)—P—PtA2 is as follows:

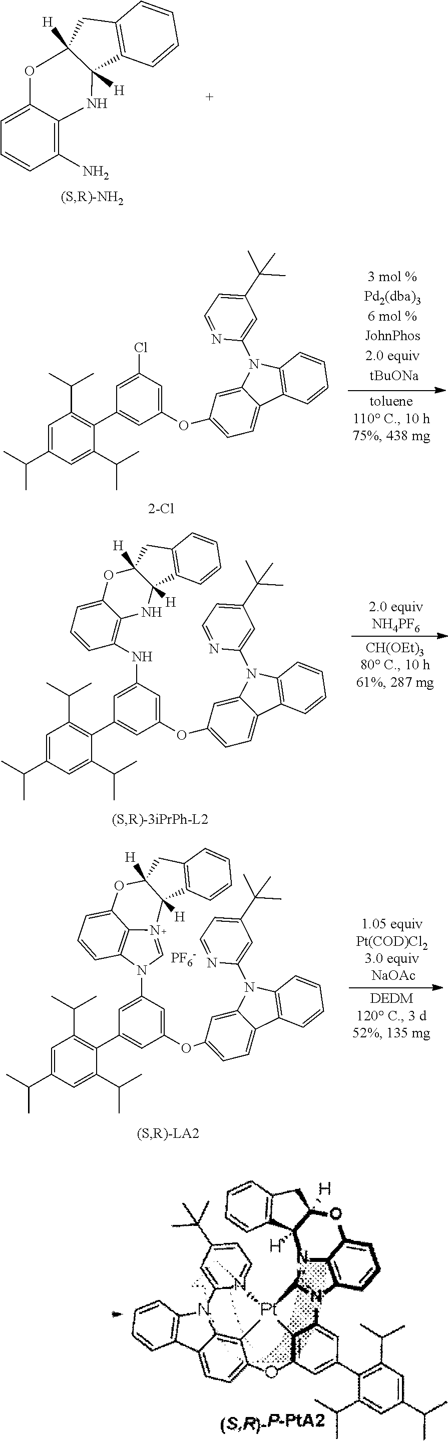

(1) Synthesis of (S,R)-3iPrPh-L2: (S,R)—NH₂ (167 mg, 0.70 mmol, 1.0 equiv), 2-Cl (441 mg, 0.70 mmol, 1.0 equiv), Pd₂(dba)₃ (19 mg, 0.021 mmol, 3 mol %), JohnPhos (13 mg, 0.042 mmol, 6 mol %) and tBuONa (135 mg, 1.4 mmol, 2.0 equiv) were added to a dry tube equipped with a magnetic stir bar. The tube was evacuated and backfilled with nitrogen, the evacuation and back fill procedure was repeated for another twice. Then toluene (6 mL) were added under a nitrogen atmosphere. The tube was sealed and the mixture was stirred at a temperature of 110° C. for 10 hours, cooled down to ambient temperature. The solvent was removed under reduced pressure and the residue was purified through column chromatography on silica gel using petroleum ether/ethyl acetate (30:1) as eluent to obtain the desired product (S,R)-3iPrPh-L2 as a yellow-green foamy solid 438 mg in 75% yield.
(2) Synthesis of (S,R)-LA2: (S,R)-3iPrPh-L2 (400 mg, 0.48 mmol, 1.0 equiv), NH₄PF₆ (157 mg, 0.96 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (4 mL) were added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using dichloromethane as eluent to obtain the desired product (S,R)-LA2 as a yellow foamy solid 287 mg in 61% yield. ¹H NMR (500 MHz, DMSO-d₆): δ(ppm) 0.92 (d, J=6.5 Hz, 6H), 1.11 (t, J=7.0 Hz, 6H), 1.19 (s, 3H), 1.20 (s, 3H), 1.31 (s, 9H), 2.53-2.60 (m, 2H), 2.85-2.90 (m, 1H), 3.32-2.34 (m, 1H), 3.57 (dd, J=16.5, 4.5 Hz, 1H), 5.43-5.45 (m, 1H), 6.40 (d, J=4.0 Hz, 1H), 7.05 (dd, J=2.0, 1.0 Hz, 1H), 7.07 (s, 2H), 7.20 (d, J=8.0 Hz, 1H), 7.24 (dd, J=8.5, 2.0 Hz, 1H), 7.30-7.35 (m, 2H), 7.39-7.43 (m, 2H), 7.45 (d, J=8.0 Hz, 2H), 7.48-7.50 (m, 2H), 7.53 (t, J=2.0 Hz, 1H), 7.59 (t, J=8.0 Hz, 1H), 7.66 (d, J=7.5 Hz, 1H), 7.69 (t, J=2.5 Hz, 1H), 7.71 (d, J=1.0 Hz, 1H), 7.73 (d, J=8.0 Hz, 1H), 8.24 (d, J=7.5 Hz, 1H), 8.34 (d, J=8.5 Hz, 1H), 8.56 (d, J=5.4 Hz, 1H), 10.49 (s, 1H).
(3) Synthesis of (S,R)—P—PtLA2: (S,R)-LA2 (250 mg, 0.25 mmol, 1.0 equiv), Pt(COD)Cl₂ (100 mg, 0.27 mmol, 1.05 equiv) and NaOAc (62 mg, 0.76 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (6 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (S,R)—P—PtA2 as a pale yellow solid 135 mg in 52% yield. ¹H NMR (500 MHz, DMSO-d₆): δ(ppm) 1.09-1.16 (m, 12H), 1.26 (s, 9H), 1.29 (d, J=7.0 Hz, 1H), 2.80-2.90 (m, 2H), 2.93-2.99 (m, 1H), 3.30-3.36 (m, 1H), 3.50 (dd, J=16.5, 2.5 Hz, 1H), 5.51 (s, 1H), 6.40 (d, J=3.5 Hz, 1H), 6.77 (d, J=1.0 Hz, 1H), 6.83 (d, J=8.0 Hz, 1H), 6.90 (t, J=7.5 Hz, 1H), 7.13 (d, J=2.5 Hz, 2H), 7.15-7.26 (m, 3H), 7.33 (d, J=8.0 Hz, 1H), 7.35-7.37 (m, 2H), 7.38-7.40 (m, 1H), 7.42-7.46 (m, 1H), 7.66 (dd, J=7.5, 3.0 Hz, 2H), 7.92 (d, J=8.5 Hz, 1H), 7.95 (d, J=8.5 Hz, 1H), 8.03 (d, J=2.0 Hz, 1H), 8.18 (dd, J=7.5, 1.0 Hz, 1H), 9.84 (d, J=6.0 Hz, 1H).

### Example 7

The synthetic route for (R,R)-M-PtA3 is as follows:

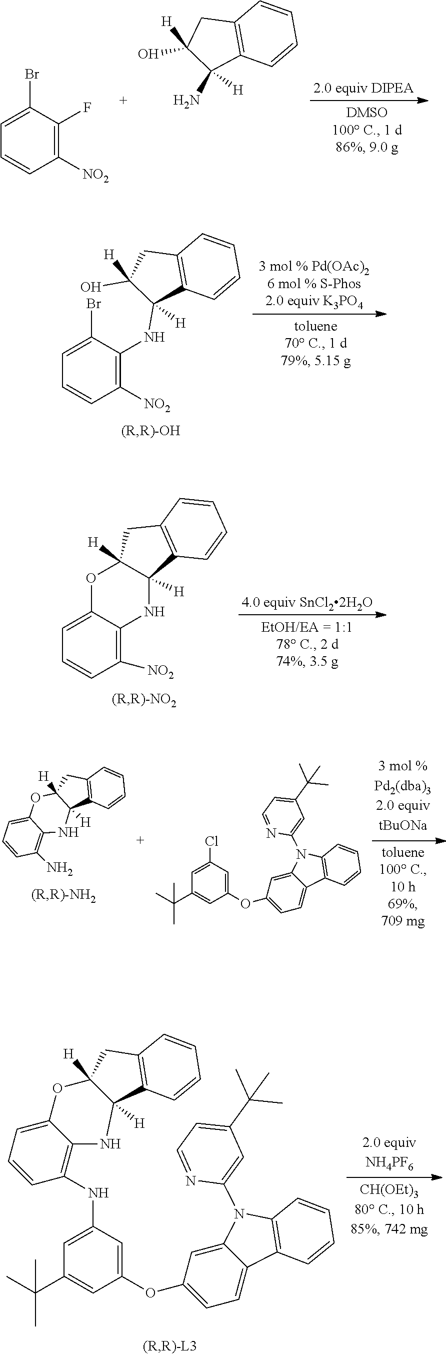

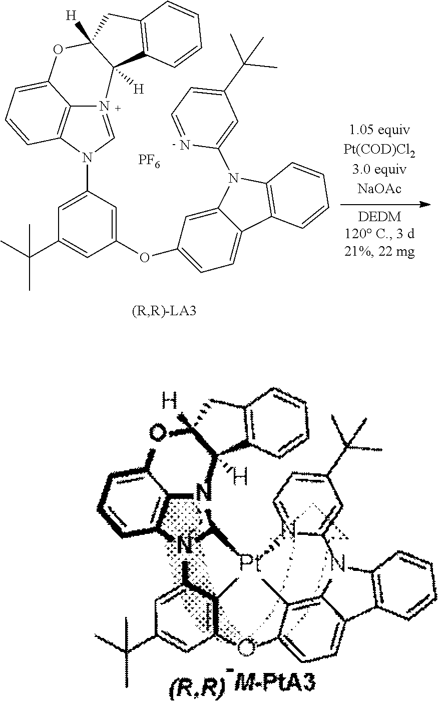

(1) Synthesis of (R,R)—OH: 1-bromo-2-fluoro-3-nitrobenzene (6.6 g, 30 mmol, 1.0 equiv) was added to a dry tube equipped with a magnetic stir bar. The tube was evacuated and backfilled with nitrogen, the evacuation and back fill procedure was repeated for another twice. Then DMSO (80 mL) and (1R,2R)-1-amino-2,3-dihydro-1H-inden-2-ol (4.48 g, 30 mmol, 1.0 equiv), N,N-Diisopropylethylamine (DIPEA) (7.75 g, 30 mmol, 2.0 equiv) were added under a nitrogen atmosphere. The tube was sealed and the mixture was stirred at a temperature of 100° C. for 1 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/dichloromethane (2:1-1:1) as eluent to obtain the desired product (R,R)—OH as a yellow solid 9.0 g in 86% yield. ¹H NMR (500 MHz, DMSO-d₆): δ(ppm) 2.63 (dd, J=15.0, 7.5 Hz, 1H), 3.08 (dd, J=15.5, 6.5 Hz, 1H), 4.11-4.17 (m, 1H), 4.82 (dd, J=10.5, 6.5 Hz, 1H), 5.28 (d, J=6.0 Hz, 1H), 6.26 (d, J=10.5 Hz, 1H), 6.99 (t, J=8.0 Hz, 1H), 7.21-7.26 (m, 3H), 7.42-7.44 (m, 1H), 7.95 (dd, J=8.0, 1.5 Hz, 1H), 8.00 (dd, J=8.5, 1.5 Hz, 1H).
(2) Synthesis of (R,R)—NO₂: (R,R)—OH (9.0 g, 25.77 mmol, 1.0 equiv), Pd(OAc)₂ (173 mg, 0.77 mmol, 3 mol %), S-Phos (636 mg, 1.55 mmol, 6 mol %) and K₃PO₄ (10.94 g, 51.54 mmol, 2.0 equiv) were added to a dry tube equipped with a magnetic stir bar. The tube was evacuated and backfilled with nitrogen, the evacuation and back fill procedure was repeated for another twice. Then toluene (150 mL) were added under a nitrogen atmosphere. The tube was sealed and the mixture was stirred at a temperature of 70° C. for 1 days, cooled down to ambient temperature. The solvent was removed under reduced pressure and the residue was purified through column chromatography on silica gel using petroleum ether/dichloromethane (2:1) as eluent to obtain the desired product (R,R)—NO₂ as a yellow solid 5.15 g in 79% yield. ¹H NMR (500 MHz, DMSO-d₆): δ(ppm) 3.03 (dd, J=13.5, 11.0 Hz, 1H), 3.29 (dd, J=13.5, 6.5 Hz, 1H), 4.24-7.29 (m, 1H), 4.50 (d, J=8.5 Hz, 1H), 6.79 (dd, J=8.5, 7.5 Hz, 1H), 7.22-7.24 (m, 1H), 7.29-7.34 (m, 2H), 7.35-7.37 (m, 1H), 7.66-7.68 (m, 1H), 7.76 (dd, J=8.0, 1.5 Hz, 1H), 8.28 (s, 1H).
(3) Synthesis of (R,R)—NH₂: (R,R)—NO₂ (5.15 g, 19.20 mmol, 1.0 equiv), SnCl₂·2H₂O (17.33 g, 76.80 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (60 mL:60 mL) were added under a nitrogen atmosphere. The flask was sealed and the mixture was stirred at a temperature of 78° C. for 2 days (The color changes from deep yellow to pale yellow and eventually to colorless), cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/dichloromethane (2:1-1:1) as eluent to obtain the desired product (R,R)—NH₂ as a yellow solid 3.5 g in 74% yield. ¹H NMR (500 MHz, DMSO-d₆): δ(ppm) 2.92 (dd, J=14.0, 11 Hz, 1H), 3.23 (dd, J=14.0, 7.0 Hz, 1H), 3.98-4.03 (m, 1H), 4.20 (dd, J=8.5, 4.0 Hz, 1H), 4.71 (s, 2H), 5.29 (d, J=3.5 Hz, 1H), 6.15 (dd, J=8.0, 1.5 Hz, 1H), 6.28 (dd, J=7.0, 1.5 Hz, 1H), 6.48 (t, J=8.0 Hz, 1H), 7.25-7.34 (m, 3H), 7.49 (d, J=7.5 Hz, 1H).
(4) Synthesis of (R,R)-L3: (R,R)—NH₂ (357 mg, 1.5 mmol, 1.0 equiv), 1-Cl (725 mg, 1.5 mmol, 1.0 equiv), Pd₂(dba)₃ (41 mg, 0.05 mmol, 3 mol %), JohnPhos (27 mg, 0.09 mmol, 6 mol %) and tBuONa (288 mg, 3.0 mmol, 2.0 equiv) were added to a dry tube equipped with a magnetic stir bar. The tube was evacuated and backfilled with nitrogen, the evacuation and back fill procedure was repeated for another twice. Then toluene (10 mL) were added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (30:1) as eluent to obtain the desired product (R,R)-L3 as a yellow solid 709 mg in 69% yield.
(5) Synthesis of (R,R)-LA3: (R,R)-L3 (709 mg, 1.04 mmol, 1.0 equiv), NH₄PF₆ (339 mg, 2.08 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (6 mL) were added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (10:1) first, then dichloromethane/ethyl acetate (10:1) as eluent to obtain the desired product (R,R)-LA3 as a brown foamy solid 742 mg in 85% yield. ¹H NMR (500 MHz, CDCl₃): δ(ppm) 1.38 (s, 9H), 1.40 (s, 9H), 3.34 (dd, J=14.0, 11.5 Hz, 1H), 3.49 (dd, J=14.0, 7.0 Hz, 1H), 4.58-4.63 (m, 1H), 5.86 (d, J=9.0 Hz, 1H), 6.99 (t, J=2.0 Hz, 1H), 7.09 (dd, J=8.5, 2.0 Hz, 1H), 7.17 (d, J=8.0 Hz, 1H), 7.30-7.32 (m, 3H), 7.37-7.45 (m, 5H), 7.52 (t, J=8.5 Hz, 1H), 7.59 (dd, J=1.5, 0.5 Hz, 1H), 7.62 (d, J=2.0 Hz, 1H), 7.68 (d, J=8.5 Hz, 1H), 7.70 (t, J=1.5 Hz, 1H), 7.90 (d, J=6.5 Hz, 1H), 8.07 (d, J=7.0 Hz, 1H), 8.10 (d, J=8.0 Hz, 1H), 8.58 (dd, J=5.5, 0.5 Hz, 1H), 9.42 (d, J=1.0 Hz, 1H).
(6) Synthesis of (R,R)-M-PtA3: (R,R)-LA3 (100 mg, 0.12 mmol, 1.0 equiv), Pt(COD)Cl₂ (49 mg, 0.13 mmol, 1.05 equiv) and NaOAc (30 mg, 0.36 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (5 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (R,R)-M-PtA3 as a pale yellow solid 22 mg in 21% yield. ¹H NMR (500 MHz, DMSO-d₆): δ(ppm) 1.13 (s, 9H), 1.47 (s, 9H), 3.23-3.29 (m, 1H), 3.35 (s, 1H), 4.53-4.58 (m, 1H), 6.11 (d, J=9.5 Hz, 1H), 6.71 (t, J=7.0 Hz, 1H), 6.96 (d, J=5.0 Hz, 1H), 7.04 (s, 1H), 7.10 (d, J=8.0 Hz, 1H), 7.17 (t, J=7.0 Hz, 1H), 7.32 (dd, J=7.0, 5.0 Hz, 2H), 7.44 (t, J=7.5 Hz, 1H), 7.49 (t, J=8.5 Hz, 1H), 7.56 (t, J=7.5 Hz, 1H), 7.63 (s, 1H), 7.93 (d, J=8.0 Hz, 1H), 7.97 (d, J=8.5 Hz, 2H), 7.99 (s, 1H), 8.22 (d, J=7.5 Hz, 1H), 8.39 (d, J=7.5 Hz, 1H), 9.50 (d, J=6.0 Hz, 1H).

### Example 8

The synthetic route for (S,S)—P—PtA3 is as follows:

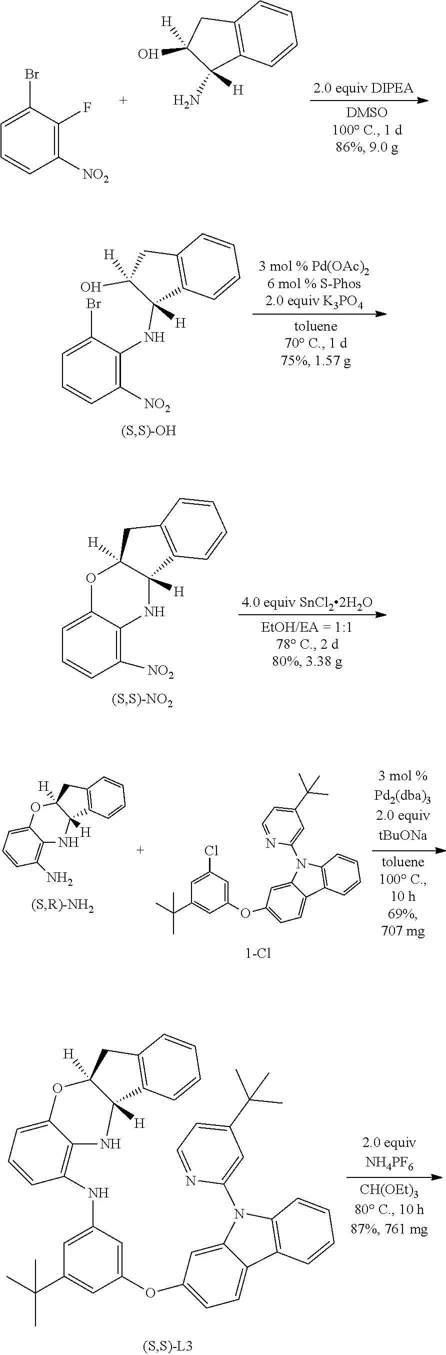

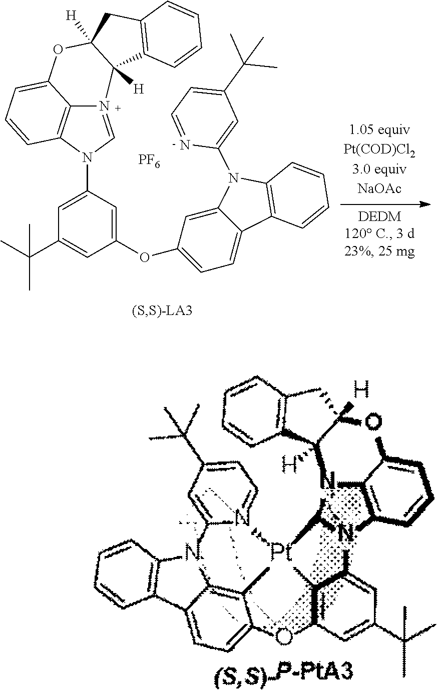

(1) Synthesis of (S,S)—OH: 1-bromo-2-fluoro-3-nitrobenzene (6.6 g, 30 mmol, 1.0 equiv) was added to a dry tube equipped with a magnetic stir bar. The tube was evacuated and backfilled with nitrogen, the evacuation and back fill procedure was repeated for another twice. Then DMSO (80 mL) and (1S, 2S)-1-amino-2,3-dihydro-1H-inden-2-ol (4.48 g, 30 mmol, 1.0 equiv), N,N-Diisopropylethylamine (DIPEA) (7.75 g, 30 mmol, 2.0 equiv) were added under a nitrogen atmosphere. The tube was sealed and the mixture was stirred at a temperature of 100° C. for 1 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/dichloromethane (2:1-1:1) as eluent to obtain the desired product (S,S)—OH as a yellow solid 9.0 g in 86% yield. ¹H NMR (500 MHz, DMSO-d₆): δ(ppm) 2.63 (dd, J=15.5, 7.5 Hz, 1H), 3.08 (dd, J=15.5, 6.0 Hz, 1H), 4.11-4.17 (m, 1H), 4.81 (dd, J=10.5, 6.5 Hz, 1H), 5.28 (d, J=6.0 Hz, 1H), 6.27 (d, J=10.5 Hz, 1H), 6.99 (t, J=8.5, 1H), 7.20-7.27 (m, 3H), 7.41-7.44 (m, 1H), 7.95 (dd, J=7.5, 1.5 Hz, 1H), 8.00 (dd, J=8.0, 1.5 Hz, 1H).
(2) Synthesis of (S,S)—NO₂: (S,S)—OH (2.73 g, 7.82 mmol, 1.0 equiv), Pd(OAc)₂ (52 mg, 0.23 mmol, 3 mol %), SPhos (193 mg, 0.47 mmol, 6 mol %) and K₃PO₄ (3.32 mg, 15.64 mmol, 2.0 equiv) were added to a dry tube equipped with a magnetic stir bar. The tube was evacuated and backfilled with nitrogen, the evacuation and back fill procedure was repeated for another twice. Then toluene (40 mL) were added under a nitrogen atmosphere. The tube was sealed and the mixture was stirred at a temperature of 70° C. for 1 days, cooled down to ambient temperature. The solvent was removed under reduced pressure and the residue was purified through column chromatography on silica gel using petroleum ether/dichloromethane (2:1) as eluent to obtain the desired product (S,S)—NO₂ as a yellow solid 1.57 g in 75% yield. ¹H NMR (500 MHz, DMSO-d₆): δ(ppm) 3.03 (dd, J=13.5, 11.0 Hz, 1H), 3.29 (dd, J=14.0, 7.0 Hz, 1H), 4.23-7.29 (m, 1H), 4.50 (d, J=8.0 Hz, δ1H), 6.79 (dd, J=8.5, 7.5 Hz, 1H), 7.22-7.24 (m, 1H), 7.29-7.34 (m, 2H), 7.35-7.37 (m, 1H), 7.66-7.67 (m, 1H), 7.76 (dd, J=8.5, 1.5 Hz, 1H), 8.28 (s, 1H).
(3) Synthesis of (S,S)—NH₂: (S,S)—NO₂ (4.75 g, 17.71 mmol, 1.0 equiv), SnCl₂·2H₂O (17.79 g, 70.84 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (60 mL:60 mL) were added under a nitrogen atmosphere. The flask was sealed and the mixture was stirred at a temperature of 78° C. for 2 days (The color changes from deep yellow to pale yellow and eventually to colorless), cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/dichloromethane (2:1-1:1) as eluent to obtain the desired product (S,S)—NH₂ as a yellow solid 3.538 g in 80% yield. ¹H NMR (500 MHz, DMSO-d₆): δ(ppm) 2.92 (dd, J=13.5, 10.5 Hz, 1H), 3.23 (dd, J=14.0, 6.5 Hz, 1H), 3.98-4.03 (m, 1H), 4.20 (dd, J=8.5, 4.0 Hz, 1H), 4.71 (s, 2H), 5.29 (d, J=4.0 Hz, 1H), 6.15 (dd, J=8.0, 1.5 Hz, 1H), 6.28 (dd, J=8.0, 1.5 Hz, 1H), 6.48 (t, J=8.0 Hz, 1H), 7.25-7.34 (m, 3H), 7.49 (d, J=7.5 Hz, 1H).
(4) Synthesis of (S,S)-L3: (S,S)—NH₂ (357 mg, 1.5 mmol, 1.0 equiv), 1-Cl (725 mg, 1.5 mmol, 1.0 equiv), Pd₂(dba)₃ (41 mg, 0.05 mmol, 3 mol %), JohnPhos (27 mg, 0.09 mmol, 6 mol %) and tBuONa (288 mg, 3.0 mmol, 2.0 equiv) were added to a dry tube equipped with a magnetic stir bar. The tube was evacuated and backfilled with nitrogen, the evacuation and back fill procedure was repeated for another twice. Then toluene (10 mL) was added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (30:1) as eluent to obtain the desired product (S,S)-L3 as a yellow solid 707 mg in 69% yield.
(5) Synthesis of (S,S)-LA3: (S,S)-L3 (761 mg, 1.03 mmol, 1.0 equiv), NH₄PF₆ (336 mg, 2.06 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (6 mL) were added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (10:1) first, then dichloromethane/ethyl acetate (10:1) as eluent to obtain the desired product (S,S)-LA3 as a brown foamy solid 761 mg in 87% yield. ¹H NMR (500 MHz, CDCl₃): δ(ppm) 1.38 (s, 9H), 1.40 (s, 9H), 3.35 (dd, J=14.0, 11.5 Hz, 1H), 3.49 (dd, J=14.0, 6.5 Hz, 1H), 4.58-4.63 (m, 1H), 5.86 (d, J=9.0 Hz, 1H), 6.99 (t, J=2.0 Hz, 1H), 7.09 (dd, J=8.5, 2.5 Hz, 1H), 7.17 (d, J=8.0 Hz, 1H), 7.30-7.33 (m, 3H), 7.37-7.45 (m, 5H), 7.52 (t, J=8.5 Hz, 1H), 7.59 (dd, J=1.5, 0.5 Hz, 1H), 7.63 (d, J=2.0 Hz, 1H), 7.68 (d, J=8.5 Hz, 1H), 7.70 (t, J=1.5 Hz, 1H), 7.90 (d, J=7.0 Hz, 1H), 8.07 (d, J=7.0 Hz, 1H), 8.10 (d, J=8.0 Hz, 1H), 8.58 (dd, J=5.5, 0.5 Hz, 1H), 9.42 (d, J=0.5 Hz, 1H).
(6) Synthesis of (S,S)—P—PtA3: (S,S)-LA3 (100 mg, 0.12 mmol, 1.0 equiv), Pt(COD)Cl₂ (49 mg, 0.13 mmol, 1.05 equiv) and NaOAc (30 mg, 0.36 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (3 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (S,S)—P—PtA3 as a pale yellow solid 25 mg in 23% yield. ¹H NMR (500 MHz, DMSO-d₆): δ(ppm) 1.13 (s, 9H), 1.47 (s, 9H), 3.29-3.33 (m, 1H), 3.38 (dd, J=13.5, 6.5 Hz, 1H), 4.53-4.59 (m, 1H), 6.11 (d, J=9.5 Hz, 1H), 6.72 (t, J=8.0 Hz, 1H), 6.96 (dd, J=6.5, 2.0 Hz, 1H), 7.04 (d, J=1.5 Hz, 1H), 7.11 (d, J=8.0 Hz, 1H), 7.18 (t, J=7.0 Hz, 1H), 7.33 (d, J=8.5 Hz, 2H), 7.44 (t, J=8.0 Hz, 1H), 7.50 (t, J=8.0 Hz, 1H), 7.55-7.63 (m, 1H), 7.63 (d, J=1.5 Hz, 2H), 7.93 (d, J=8.5 Hz, 1H), 7.97 (d, J=8.5 Hz, 2H), 8.00 (d, J=2.0 Hz, 1H), 8.22 (d, J=7.5 Hz, 1H), 8.40 (d, J=8.0 Hz, 1H), 9.51 (d, J=6.5 Hz, 1H).

### Example 9

The synthetic route for (R)-iPr-M-PtA4 is as follows:

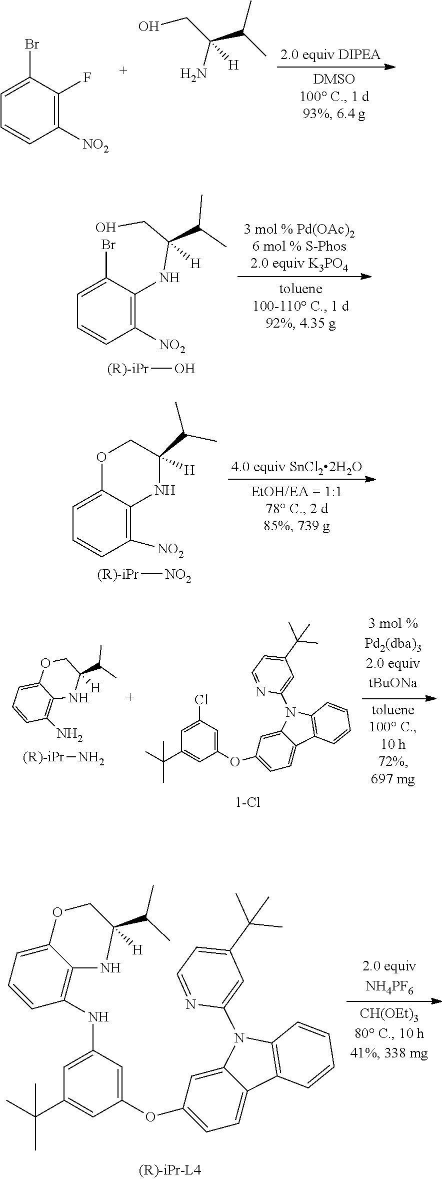

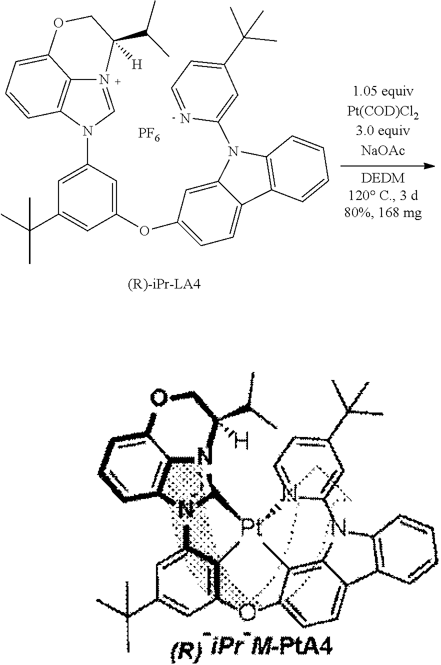

(1) Synthesis of (R)-iPr-OH: 1-bromo-2-fluoro-3-nitrobenzene (5.0 g, 22.73 mmol, 1.0 equiv), (R)-2-amino-3-methylbutan-1-ol (2.34 g, 22.73 mmol, 1.0 equiv) were added to a dry tube equipped with a magnetic stir bar. The tube was evacuated and backfilled with nitrogen, the evacuation and back fill procedure was repeated for another twice. Then DMSO (40 mL) and N,N-Diisopropylethylamine (DIPEA) (5.88 g, 45.46 mmol, 2.0 equiv) were added under a nitrogen atmosphere. The tube was sealed and the mixture was stirred at a temperature of 100° C. for 1 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/dichloromethane (6:1-4:1) as eluent to obtain the desired product (R)-iPr-OH as a yellow oily liquid 6.4 g in 93% yield.
(2) Synthesis of (R)-iPr—NO₂: Pd(OAc)₂ (473 mg, 0.64 mmol, 3 mol %), SPhos (521 mg, 1.27 mmol, 6 mol %) and K₃PO₄ (8.98 g, 42.34 mmol, 2.0 equiv) were added to a dry tube equipped with a magnetic stir bar. The tube was evacuated and backfilled with nitrogen, the evacuation and back fill procedure was repeated for another twice. Then (R)-iPr-OH (6.42 g, 21.17 mmol, 1.0 equiv) and toluene (60 mL) were added under a nitrogen atmosphere. The tube was sealed and the mixture was stirred at a temperature of 100-110° C. for 1 days, cooled down to ambient temperature. The solvent was removed under reduced pressure and the residue was purified through column chromatography on silica gel using petroleum ether/ethyl acetate (20:1) as eluent to obtain the desired product (R)-iPr—NO₂ as a orange-red solid 4.35 g in 92% yield.
(3) Synthesis of (R)-iPr-NH₂: (R)-iPr—NO₂ (1 g, 4.5 mmol, 1.0 equiv), SnCl₂·2H₂O (4.06 g, 18.0 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (40 mL:40 mL) were added under a nitrogen atmosphere. The flask was sealed and the mixture was stirred at a temperature of 78° C. for 2 days, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/ethyl acetate (10:1-5:1) as eluent to obtain the desired product (R)-iPr-NH₂ as a white oily liquid 739 mg in 85% yield.
(4) Synthesis of (R)-iPr-L4: (R)-iPr-NH₂ (288 mg, 1.5 mmol, 1.0 equiv), 1-Cl (724 mg, 1.5 mmol, 1.0 equiv), Pd₂(dba)₃ (52 mg, 0.045 mmol, 3 mol %), JohnPhos (27 mg, 0.09 mmol, 6 mol %) and tBuONa (288 mg, 3.0 mmol, 2.0 equiv) were added to a dry tube equipped with a magnetic stir bar. The tube was evacuated and backfilled with nitrogen, the evacuation and back fill procedure was repeated for another twice. Then toluene (6 mL) were added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (30:1) as eluent to obtain the desired product (R)-iPr-L4 as a yellow foamy solid 697 mg in 72% yield.
(5) Synthesis of (R)-iPr-LA4: (R)-iPr-L4 (658 mg, 1.03 mmol, 1.0 equiv), NH₄PF₆ (336 mg, 2.06 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (10 mL) were added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (10:1) first, then dichloromethane/ethyl acetate (20:1) as eluent to obtain the desired product (R)-iPr-LA4 as a yellow foamy solid 338 mg in 41% yield. ¹H NMR (500 MHz, DMSO-d₆): δ(ppm) 1.00-1.08 (m, 6H), 1.30 (s, 9H), 1.36 (s, 9H), 2.34-2.42 (m, 1H), 4.51 (dd, J=12.5, 3.0 Hz, 1H), 4.73-4.76 (m, 1H), 4.78 (dd, J=12.0, 3.5 Hz, 1H), 7.17-7.23 (m, 2H), 7.27 (t, J=2.0 Hz, 1H), 7.32-7.37 (m, 2H), 7.44-7.49 (m, 3H), 7.50 (d, J=2.0 Hz, 1H), 7.53 (t, J=8.0 Hz, 1H), 7.58 (t, J=2.0 Hz, 1H), 7.67 (d, J=1.5 Hz, 1H), 7.74 (d, J=8.5 Hz, 1H), 8.24 (d, J=7.5 Hz, 1H), 8.32 (d, J=8.0 Hz, 1H), 8.58 (d, J=5.5 Hz, 1H), 10.21 (s, 1H).
(6) Synthesis of (R)-iPr-M-PtA4: (R)-iPr-LA4 (200 mg, 0.25 mmol, 1.0 equiv), Pt(COD)Cl₂ (98 mg, 0.26 mmol, 1.05 equiv) and NaOAc (62 mg, 0.75 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (15 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (R)-iPr-M-PtA4 as a pale yellow solid 168 mg in 80% yield. ¹H NMR (500 MHz, DMSO-d₆): δ(ppm) 0.70 (d, J=7.0 Hz, 3H), 0.72 (d, J=7.0 Hz, 3H), 1.28 (s, 9H), 1.45 (s, 9H), 2.17 (m, 1H), 4.47-4.54 (m, 1H), 4.67-4.73 (m, 1H), 4.86 (d, J=12.0 Hz, 1H), 6.93 (d, J=8.0 Hz, 1H), 7.01 (d, J=1.5 Hz, 1H), 7.21 (dd, J=6.5, 1.5 Hz, 1H), 7.28 (d, J=8.5 Hz, 1H), 7.37-7.42 (m, 2H), 7.46 (d, J=1.0 Hz, 1H), 7.47-7.51 (m, 1H), 7.67 (d, J=8.0 Hz, 1H), 7.89 (d, J=8.5 Hz, 1H), 8.03 (d, J=8.5 Hz, 1H), 8.11 (d, J=1.5 Hz, 1H), 8.15 (d, J=7.5 Hz, 1H), 9.72 (d, J=6.5 Hz, 1H).

### Example 10

The synthetic route for (S)-iPr—P—PtA4 is as follows:

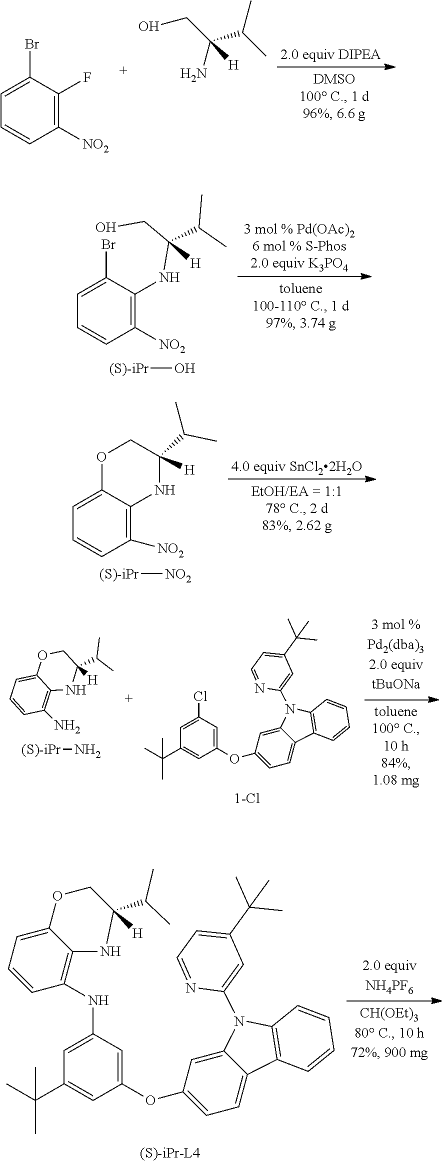

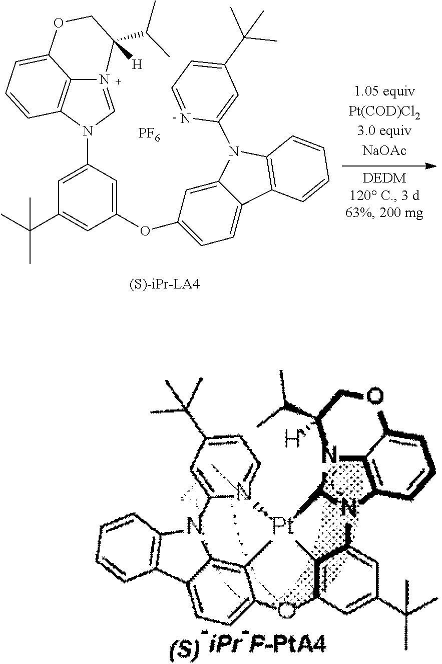

(1) Synthesis of (S)-iPr-OH: 1-bromo-2-fluoro-3-nitrobenzene (5.0 g, 22.73 mmol, 1.0 equiv), (S)-2-amino-3-methylbutan-1-ol (2.34 g, 22.73 mmol, 1.0 equiv) were added to a dry tube equipped with a magnetic stir bar. The tube was evacuated and backfilled with nitrogen, the evacuation and back fill procedure was repeated for another twice. Then DMSO (40 mL) and N,N-Diisopropylethylamine (DIPEA) (5.88 g, 45.46 mmol, 2.0 equiv) were added under a nitrogen atmosphere. The tube was sealed and the mixture was stirred at a temperature of 100° C. for 1 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/dichloromethane (6:1-4:1) as eluent to obtain the desired product (S)-iPr-OH as a yellow oily liquid 6.6 g in 96% yield. ¹H NMR (500 MHz, DMSO-d₆): δ(ppm) 0.86 (d, J=7.0 Hz, 3H), 0.93 (d, J=6.5 Hz, 3H), 1.87-1.97 (m, 1H), 3.40 (dt, J=9.5, 5.0 Hz, 1H), 3.44-3.55 (m, 2H), 4.74 (t, J=4.5 Hz, 1H), 6.20 (d, J=10.5 Hz, 1H), 6.85 (t, J=8.0 Hz, 1H), 7.85 (dd, J=8.0, 1.5 Hz, 1H), 7.90 (dd, J=8.5, 1.5 Hz, 1H).
(2) Synthesis of (S)-iPr—NO₂: Pd(OAc)₂ (115 mg, 0.51 mmol, 3 mol %), SPhos (423 mg, 1.03 mmol, 6 mol %) and K₃PO₄ (7.27 g, 34.24 mmol, 2.0 equiv) were added to a dry tube equipped with a magnetic stir bar. The tube was evacuated and backfilled with nitrogen, the evacuation and back fill procedure was repeated for another twice. Then (S)-iPr-OH (5.20 g, 17.12 mmol, 1.0 equiv) and toluene (60 mL) were added under a nitrogen atmosphere. The tube was sealed and the mixture was stirred at a temperature of 100-110° C. for 1 days, cooled down to ambient temperature. The solvent was removed under reduced pressure and the residue was purified through column chromatography on silica gel using petroleum ether/dichloromethane (20:1) as eluent to obtain the desired product (S)-iPr—NO₂ as a orange-red solid 3.74 g in 97% yield. ¹H NMR (500 MHz, CDCl₃): δ(ppm) 1.05 (d, J=7.0 Hz, 3H), 1.10 (d, J=6.5 Hz, 3H), 1.86-1.93 (m, 1H), 3.33-3.37 (m, 1H), 4.00 (dd, J=11.0, 6.5 Hz, 1H), 4.23-4.26 (m, 1H), 6.53 (dd, J=9.0, 7.5 Hz, 1H), 6.96-6.97 (m, 1H), 7.76 (dd, J=8.5, 1.5 Hz, 1H), 8.08 (s, 1H),
(3) Synthesis of (S)-iPr-NH₂: (S)-iPr—NO₂ (3.74 g, 16.82 mmol, 1.0 equiv), SnCl₂·2H₂O (15.18 g, 62.70 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (40 mL:40 mL) were added under a nitrogen atmosphere. The flask was sealed and the mixture was stirred at a temperature of 78° C. for 2 days, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/ethyl acetate (10:1-5:1) as eluent to obtain the desired product (S)-iPr-NH₂ as a white oily liquid 2.62 g in 83% yield. ¹H NMR (500 MHz, DMSO-d₆): δ(ppm) 0.95 (d, J=7.0 Hz, 3H), 1.00 (d, J=7.0 Hz, 3H), 1.68-1.78 (m, 1H), 2.95-2.99 (m, 1H), 3.77 (dd, J=10.5, 7.5 Hz, 1H), 4.08 (dd, J=10.5, 2.0 Hz, 1H), 4.26 (d, J=3.0 Hz, 1H), 4.59 (s, 2H), 6.02 (dd, J=8.0, 1.5 Hz, 1H), 6.15 (dd, J=8.0, 1.5 Hz, 1H), 6.30 (t, J=8.0 Hz, 1H).
(4) Synthesis of (S)-iPr-L4: (S)-iPr-NH₂ (385 mg, 2.0 mmol, 1.0 equiv), 1-Cl (966 mg, 2.0 mmol, 1.0 equiv), Pd₂(dba)₃ (55 mg, 0.06 mmol, 3 mol %), JohnPhos (36 mg, 0.12 mmol, 6 mol %) and tBuONa (384 mg, 4.0 mmol, 2.0 equiv) were added to a dry tube equipped with a magnetic stir bar. The tube was evacuated and backfilled with nitrogen, the evacuation and back fill procedure was repeated for another twice. Then toluene (15 mL) were added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (20:1) as eluent to obtain the desired product (S)-iPr-L4 as a yellow foamy solid 1.08 g in 84% yield. ¹H NMR (500 MHz, DMSO-d₆): δ(ppm) 0.88 (t, J=6.5 Hz, 6H), 1.20 (s, 9H), 1.30 (s, 9H), 1.63-1.72 (m, 1H), 2.97-3.00 (m, 1H), 3.80 (dd, J=10.5, 7.0 Hz, 1H), 4.06 (dd, J=10.5, 2.0 Hz, 1H), 4.55 (d, J=2.0 Hz, 1H), 6.24 (t, J=2.0 Hz, 1H), 6.34 (t, J=8.0 Hz, 1H), 6.38 (dd, J=8.0, 1.5 Hz, 1H), 6.49 (t, J=2.0 Hz, 1H), 6.58 (dd, J=8.0, 2.0 Hz, 1H), 6.64 (t, J=1.5 Hz, 1H), 7.03 (dd, J=8.5, 2.0 Hz, 1H), 7.27 (s, 1H), 7.30-7.33 (m, 2H), 7.40-7.47 (m, 1H), 7.46 (dd, J=5.5, 2.0 Hz, 1H), 7.60 (d, J=1.0 Hz, 1H), 7.73 (d, J=8.5 Hz, 1H), 8.19 (d, J=7.5 Hz, 1H), 8.21 (d, J=8.5 Hz, 1H), 8.57 (d, J=5.5 Hz, 1H).
(5) Synthesis of (S)-iPr-LA4: (S)-iPr-L4 (1.0 g, 1.57 mmol, 1.0 equiv), NH₄PF₆ (512 mg, 3.14 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (5 mL) were added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (10:1) first, then dichloromethane/ethyl acetate (20:1) as eluent to obtain the desired product (S)-iP-LA4 as a yellow foamy solid 900 mg in 72% yield. ¹H NMR (500 MHz, DMSO-d₆): δ(ppm) 1.04 (dd, J=8.5, 7.0 Hz, 6H), 1.30 (s, 9H), 1.36 (s, 9H), 2.34-2.51 (m, 1H), 4.51 (dd, J=12.5, 3.0 Hz, 1H), 4.73-4.76 (m, 1H), 4.79 (dd, J=12.0, 3.0 Hz, 1H), 7.18-7.22 (m, 2H), 7.27 (t, J=2.5 Hz, 1H), 7.33-7.36 (m, 2H), 7.45-7.49 (m, 3H), 7.51 (d, J=2.5 Hz, 1H), 7.54 (t, J=8.0 Hz, 1H), 7.58 (t, J=1.5 Hz, 1H), 7.68 (d, J=1.0 Hz, 1H), 7.74 (d, J=8.5 Hz, 1H), 8.24 (d, J=8.0 Hz, 1H), 8.33 (d, J=8.5 Hz, 1H), 8.58 (d, J=5.0 Hz, 1H), 10.22 (s, 1H).
(6) Synthesis of (S)-iPr—P—PtA4: (S)-iPr-LA4 (300 mg, 0.38 mmol, 1.0 equiv), Pt(COD)Cl₂ (150 mg, 0.40 mmol, 1.05 equiv) and NaOAc (94 mg, 1.14 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (23 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (S)-iPr—P—PtA4 as a pale yellow solid 200 mg in 63% yield. ¹H NMR (500 MHz, DMSO-d₆): δ(ppm) 0.67 (dd, J=9.5, 7.0 Hz, 6H), 1.15 (s, 9H), 1.46 (s, 9H), 2.08-2.18 (m, 1H), 4.27 (d, J=11.0 Hz, 1H), 4.62 (d, J=3.5 Hz, 1H), 4.81 (d, J=12 Hz, 1H), 6.92 (d, J=8.0 Hz, 1H), 7.03-7.05 (m, 2H), 7.29 (d, J=8.0 Hz, 1H), 7.35-7.40 (m, 3H), 7.45-7.48 (m, 1H), 7.55 (d, J=8.5 Hz, 1H), 7.89 (d, J=8.0 Hz, 1H), 7.99 (d, J=8.5 Hz, 1H), 8.01 (d, J=1.5 Hz, 1H), 8.14 (dd, J=7.5, 0.5 Hz, 1H), 9.78 (d, J=6.0 Hz, 1H).

### Example 11

The synthetic route for (S)-2MeiPr—P—PtA5 is as follows:

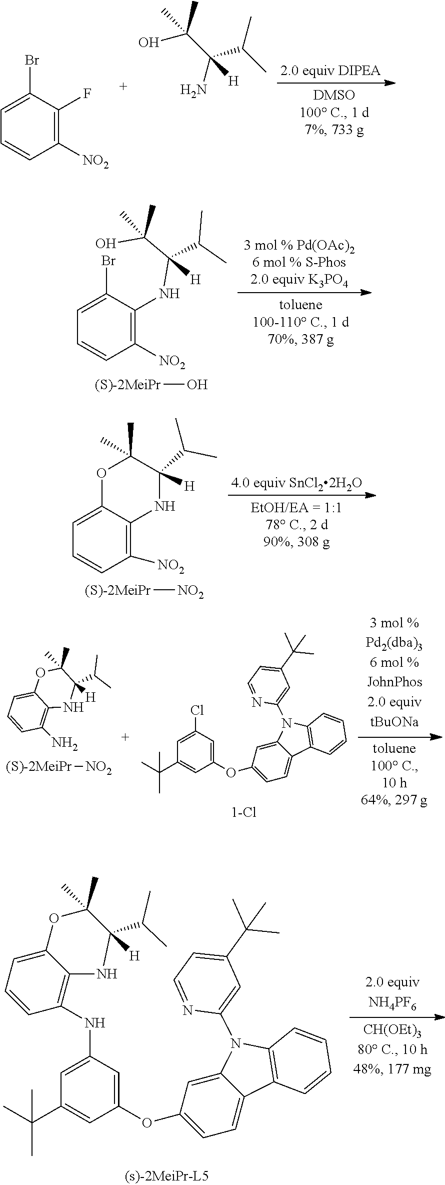

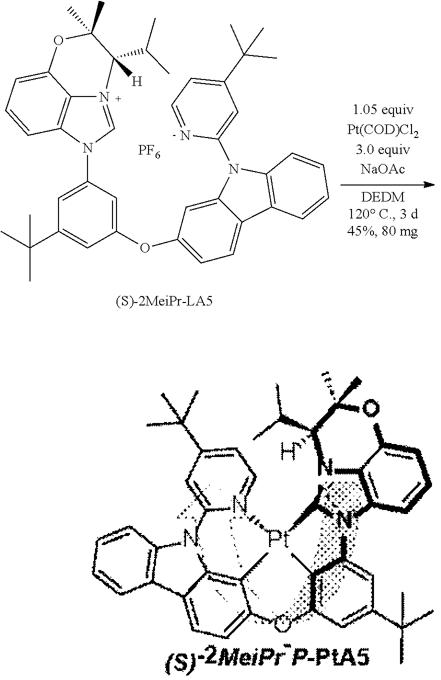

(1) Synthesis of (S)-2MeiPr—OH: 1-bromo-2-fluoro-3-nitrobenzene (6.82 g, 31 mmol, 1.5 equiv), (S)-3-amino-2,4-dimethylpentan-2-ol (6.2 g, 47 mmol, 1.0 equiv) were added to a dry tube equipped with a magnetic stir bar. The tube was evacuated and backfilled with nitrogen, the evacuation and back fill procedure was repeated for another twice. Then DMSO (40 mL) and N,N-Diisopropylethylamine (DIPEA) (8.01 g, 62 mmol, 2.0 equiv) were added under a nitrogen atmosphere. The tube was sealed and the mixture was stirred at a temperature of 100° C. for 1 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate (50:1-20:1) as eluent to obtain the desired product (S)-2MeiPr—OH as a red oily liquid 733 mg in 7% yield.
(2) Synthesis of (S)-2MeiPr—NO₂: Pd(OAc)₂ (15 mg, 0.06 mmol, 3 mol %), SPhos (54 mg, 0.13 mmol, 6 mol %) and K₃PO₄ (938 mg, 4.42 mmol, 2.0 equiv) were added to a dry tube equipped with a magnetic stir bar. The tube was evacuated and backfilled with nitrogen, the evacuation and back fill procedure was repeated for another twice. Then (S)-2MeiPr—OH (733 mg, 2.21 mmol, mmol, 1.0 equiv) and toluene (60 mL) were added under a nitrogen atmosphere. The tube was sealed and the mixture was stirred at a temperature of 100-110° C. for 1 days, cooled down to ambient temperature. The solvent was removed under reduced pressure and the residue was purified through column chromatography on silica gel using petroleum ether/dichloromethane (20:1) as eluent to obtain the desired product (S)-iPr—NO₂ as a orange-red solid 387 mg in 70% yield.
(3) Synthesis of (S)-2MeiPr-NH₂: (S)-2MeiPr—NO₂ (387 mg, 1.55 mmol, 1.0 equiv), SnCl₂·2H₂O (1.4 g, 6.2 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (40 mL:40 mL) were added under a nitrogen atmosphere. The flask was sealed and the mixture was stirred at a temperature of 78° C. for 2 days, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/dichloromethane (2:1) first, then dichloromethane as eluent to obtain the desired product (S)-2MeiPr-NH₂ as a white oily liquid 308 mg in 90% yield.
(4) Synthesis of (S)-2MeiPr-L5: (S)-2MeiPr-NH₂ (159 mg, 0.70 mmol, 1.0 equiv), 1-Cl (338 mg, 0.70 mmol, 1.0 equiv), Pd₂(dba)₃ (19 mg, 0.02 mmol, 3 mol %), JohnPhos (13 mg, 0.04 mmol, 6 mol %) and tBuONa (134 mg, 1.40 mmol, 2.0 equiv) were added to a dry tube equipped with a magnetic stir bar. The tube was evacuated and backfilled with nitrogen, the evacuation and back fill procedure was repeated for another twice. Then toluene (5 mL) were added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (30:1) as eluent to obtain the desired product (S)-2MeiPr-L5 as a white foamy solid 297 mg in 64% yield.
(5) Synthesis of (S)-2MeiPr-LA5: (S)-2MeiPr-L5 (297 mg, 0.45 mmol, 1.0 equiv), NH₄PF₆ (145 mg, 0.90 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (6 mL) were added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (10:1) first, then dichloromethane/ethyl acetate (20:1) as eluent to obtain the desired product (S)-2MeiPr-LA5 as a yellow foamy solid 177 mg in 48% yield.
(6) Synthesis of (S)-2MeiPr—P—PtA5: (S)-2MeiPr-LA5 (177 mg, 0.22 mmol, 1.0 equiv), Pt(COD)Cl₂ (84 mg, 0.23 mmol, 1.05 equiv) and NaOAc (53 mg, 0.65 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (13 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (10:1-1:1) as eluent to obtain the desired product (S)-2MeiPr—P—PtA5 as a yellow solid 85 mg in 45% yield. ¹H NMR (500 MHz, DMSO-d₆): δ 0.30 (d, J=7.0 Hz, 3H), 0.79-0.88 (m, 1H), 0.97 (d, J=7.0 Hz, 3H), 1.37 (s, 9H), 1.45 (s, 9H), 1.58 (s, 3H), 1.67 (s, 3H), 4.51 (d, J=2.5 Hz, 1H), 6.90 (d, J=8.0 Hz, 1H), 7.00 (d, J=1.5 Hz, 1H), 7.28 (d, J=8.0 Hz, 1H), 7.38-7.44 (m, 2H), 7.46 (dd, J=6.5, 2.0 Hz, 1H), 7.47-7.52 (m, 1H), 7.57 (d, J=1.5 Hz, 1H), 7.77 (d, J=8.5 Hz, 1H), 7.90 (d, J=8.5 Hz, 1H), 8.07 (d, J=8.5 Hz, 1H), 8.13-8.19 (m, 2H), 9.40 (d, J=6.5 Hz, 1H).

### Example 12

The synthetic route for (R)-iBu-M-PtA6 is as follows:

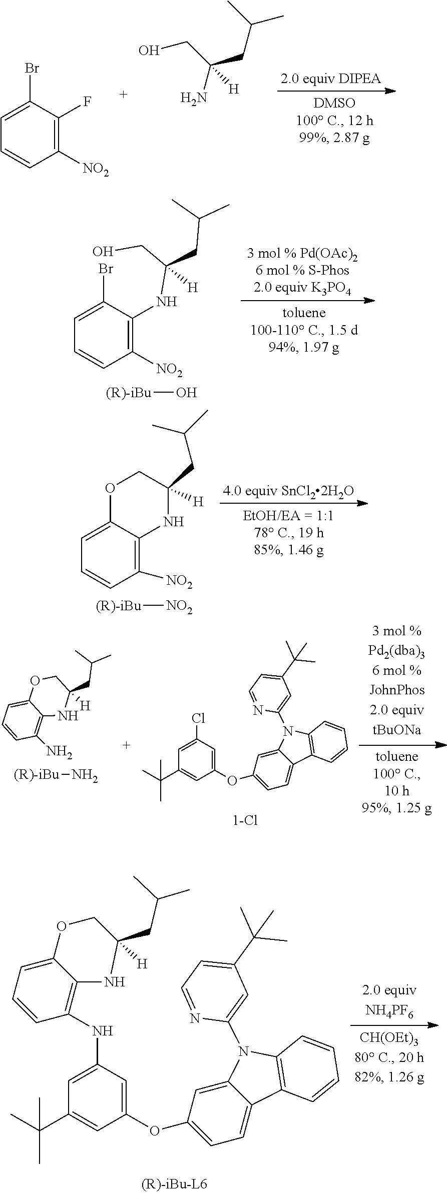

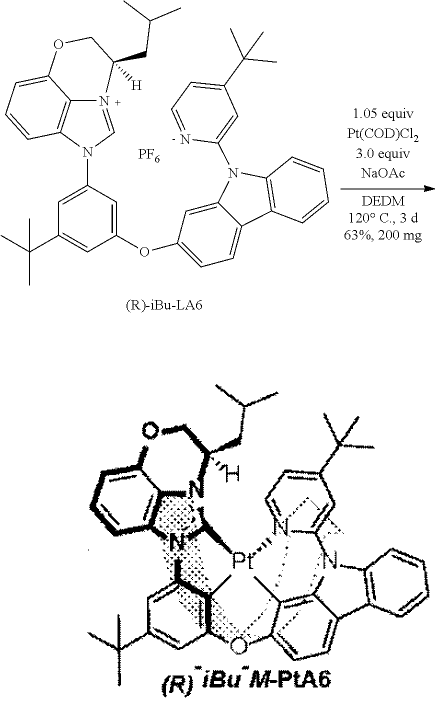

(1) Synthesis of (R)-iBu-OH: 1-bromo-2-fluoro-3-nitrobenzene (2 g, 9.09 mmol, 1.0 equiv) was added to a dry tube equipped with a magnetic stir bar. The tube was evacuated and backfilled with nitrogen, the evacuation and back fill procedure was repeated for another twice. Then (R)-2-amino-4-methylpentan-1-ol (1.28 g, 10.91 mmol, 1.2 equiv), N,N-Diisopropylethylamine (DIPEA) (2.35 g, 18.18 mmol, 2.0 equiv) and DMSO (16 mL) were added under a nitrogen atmosphere. The tube was sealed and the mixture was stirred at a temperature of 100° C. for 12 hours, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate (20:1-10:1) as eluent to obtain the desired product (R)-iBu-OH as a red oily liquid 2.87 g in 99% yield. ¹H NMR (500 MHz, CDCl₃): δ(ppm) 0.89 (t, J=7.0 Hz, 6H), 1.39-1.49 (m, 2H), 1.61-1.66 (m, 1H), 1.72 (dd, J=6.5, 4.5 Hz, 1H), 3.55-3.59 (m, 1H), 3.65-3.70 (m, 1H), 3.95-4.05 (m, 1H), 6.21 (d, J=10.5 Hz, 1H), 6.79 (t, J=7.5 Hz, 1H), 7.72 (dd, J=8.0, 1.5 Hz, 1H), 7.92 (dd, J=10, 2 Hz, 1H).
(2) Synthesis of (R)-iBu-NO₂: Pd(OAc)₂ (60 mg, 0.27 mmol, 3 mol %), SPhos (219 mg, 0.53 mmol, 6 mol %) and K₃PO₄ (3.77 g, 17.78 mmol, 2.0 equiv) were added to a dry tube equipped with a magnetic stir bar. The tube was evacuated and backfilled with nitrogen, the evacuation and back fill procedure was repeated for another twice. Then (R)-iBu-OH (2.82 g, 8.89 mmol, 1.0 equiv) and toluene (30 mL) were added under a nitrogen atmosphere. The tube was sealed and the mixture was stirred at a temperature of 100-110° C. for 1.5 days, cooled down to ambient temperature. The solvent was removed under reduced pressure and the residue was purified through column chromatography on silica gel using petroleum ether/ethyl acetate (30:1) as eluent to obtain the desired product (R)-iBu-NO₂ as a red oily liquid 1.97 g in 94% yield. ¹H NMR (500 MHz, CDCl₃): δ(ppm) 1.01 (dd, J=6.5, 4.0 Hz, 6H), 1.39-1.45 (m, 1H), 1.48-1.55 (m, 1H), 1.78-1.86 (m, 1H), 3.66-3.73 (m, 1H), 3.83 (dd, J=10.5, 7 Hz, 1H), 4.22-4.25 (m, 1H), 6.54 (dd, J=9.0, 7.5 Hz, 1H), 6.97-6.99 (m, 1H), 7.76 (dd, J=9, 1.5 Hz, 1H), 7.94 (s, 1H).
(3) Synthesis of (R)-iBu-NH₂: SnCl₂·2H₂O (7.96 g, 35.28 mmol, 4.0 equiv) was added to a three-necked flask equipped with a magnetic stir bar. The flask was evacuated and backfilled with nitrogen, the evacuation and back fill procedure was repeated for another twice. Then (R)-iBu-NO₂ (1.95 g, 8.25 mmol, 1.0 equiv), ethanol and ethyl acetate (1:1) were added under a nitrogen atmosphere. The flask was sealed and the mixture was stirred at a temperature of 78° C. for 19 hours, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/ethyl acetate (20:1-1:1) as eluent to obtain the desired product (R)-iBu-NH₂ as a yellow oily liquid 1.46 g in 85% yield. ¹H NMR (500 MHz, CDCl₃): δ (ppm) 0.98 (t, J=6.5 Hz, 6H), 1.31-1.36 (m, 1H), 1.39-1.45 (m, 1H), 1.79-1.87 (m, 1H), 2.68 (s, 3H), 3.34-3.43 (m, 1H), 3.72 (dd, J=10.5, 8.0 Hz, 1H), 4.19 (dd, J=10.5, 2.5 Hz, 1H), 6.34 (dd, J=8.0, 1.5 Hz, 1H), 6.39 (dd, J=8.5, 1.0 Hz, 1H), 6.62 (t, J=8.0 Hz, 1H).
(4) Synthesis of (R)-iBu-L6: 1-Cl (966 mg, 2.0 mmol, 1.0 equiv), Pd₂(dba)₃ (55 mg, 0.06 mmol, 3 mol %), JohnPhos (38 mg, 0.12 mmol, 6 mol %) and tBuONa (384 mg, 4.0 mmol, 2.0 equiv) were added to a dry tube equipped with a magnetic stir bar. The tube was evacuated and backfilled with nitrogen, the evacuation and back fill procedure was repeated for another twice. Then (R)-iBu-NH₂ (412 mg, 2.0 mmol, 1.0 equiv) and toluene (10 mL) were added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (30:1-20:1) as eluent to obtain the desired product (R)-iBu-L6 as a yellow foamy solid 1.25 g in 95% yield.
(5) Synthesis of (R)-iBu-LA6: (R)-iBu-L6 (1.25 g, 1.91 mmol, 1.0 equiv), NH₄PF₆ (624 mg, 3.83 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (8 mL) was added under a nitrogen atmosphere. The tube was sealed and the mixture was stirred at a temperature of 80° C. for 20 hours, cooled down to ambient temperature. The solvent was removed under reduced pressure and the residue was purified through column chromatography on silica gel using petroleum ether/ethyl acetate (10:1) first, then dichloromethane/ethyl acetate (20:1) as eluent to obtain the desired product (R)-iBu-LA6 as a yellow foamy solid 1.26 g in 82% yield. ¹H NMR (500 MHz, DMSO-d₆): δ(ppm) 0.96 (d, J=6.5 Hz, 3H), 1.00 (d, J=6.5 Hz, 3H), 1.29 (s, 9H), 1.35 (s, 9H), 1.71-1.78 (m, 1H), 1.79-1.87 (m, 1H), 1.93-1.98 (m, 1H), 4.49 (dd, J=12.0, 5.6 Hz, 1H), 4.67 (dd, J=12.0, 3.0 Hz, 1H), 4.89-4.94 (m, 1H), 7.20 (dd, 9.0, 1.5 Hz, 2H), 7.25 (t, J=2 Hz, 1H), 7.32-7.36 (m, 2H), 7.44-7.48 (m, 3H), 7.49 (d, J=2.0 Hz, 1H), 7.54 (t, J=9 Hz, 1H), 7.56 (d, J=1.5 Hz, 1H), 7.67 (d, J=1.0 Hz, 1H), 7.74 (d, J=8.0 Hz, 1H), 8.24 (d, J=7.5 Hz, 1H), 8.32 (d, J=8.5 Hz, 1H), 8.58 (dd, J=5.0, 0.5 Hz 1H), 10.19 (s, 1H).
(6) Synthesis of (R)-iBu-M-PtA6: (R)-iBu-LA6 (300 mg, 0.37 mmol, 1.0 equiv), Pt(COD)Cl₂ (146 mg, 0.39 mmol, 1.05 equiv) and NaOAc (91 mg, 1.11 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (22 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (R)-iBu-M-PtA6 as a pale yellow solid 200 mg in 63% yield. ¹H NMR (500 MHz, DMSO-d₆): δ(ppm) 0.39 (d, J=6.5 Hz, 3H), 0.60 (d, J=6.5 Hz, 3H), 0.81-0.87 (m, 1H), 1.33 (s, 9H), 1.45 (s, 9H), 1.62-1.67 (m, 1H), 1.82-1.91 (m, 1H), 4.63 (d, J=12.0 Hz, 1H), 4.64 (d, J=10.5 Hz, 1H), 4.91-5.00 (m, 1H), 6.94 (d, J=8.0 Hz, 1H), 7.00 (d, J=1.5 Hz, 1H), 7.28 (d, J=8.0 Hz, 1H), 7.31 (dd, J=6.5, 2.0 Hz, 1H), 7.38-7.42 (m, 2H), 7.47-7.55 (m, 2H), 7.72 (d, J=8.5 Hz, 1H), 7.90 (d, J=8.0 Hz, 1H), 7.92 (d, J=8.0 Hz, 1H), 8.12 (d, J=2.0 Hz, 1H), 8.17 (d, J=7.0 Hz, 1H), 9.68 (d, J=6.0 Hz, 1H).

### Example 13

The synthetic route for (S)-iBu-P—PtA6 is as follows:

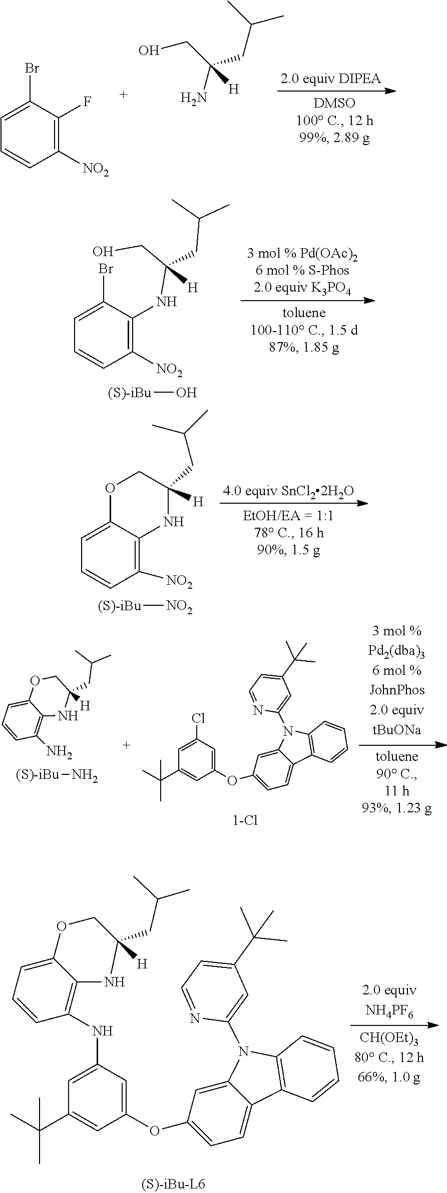

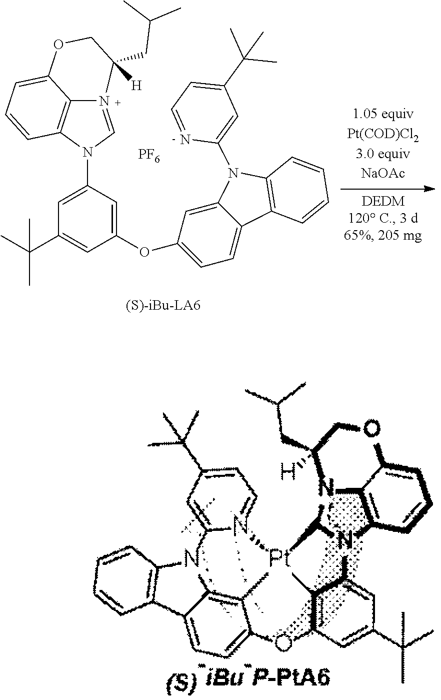

(1) Synthesis of (S)-iBu-OH: 1-bromo-2-fluoro-3-nitrobenzene (2 g, 9.09 mmol, 1.0 equiv) was added to a dry tube equipped with a magnetic stir bar. The tube was evacuated and backfilled with nitrogen, the evacuation and back fill procedure was repeated for another twice. Then (S)-2-amino-4-methylpentan-1-ol (1.28 g, 10.91 mmol, 1.2 equiv), N,N-Diisopropylethylamine (DIPEA) (2.35 g, 18.18 mmol, 2.0 equiv) and DMSO (16 mL) were added under a nitrogen atmosphere. The tube was sealed and the mixture was stirred at a temperature of 100° C. for 12 hours, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate (20:1-10:1) as eluent to obtain the desired product (S)-iBu-OH as a red oily liquid 2.89 g in 99% yield. ¹H NMR (500 MHz, CDCl₃): δ(ppm) 0.89 (t, J=7.0 Hz, 6H), 1.39-1.48 (m, 2H), 1.60-1.65 (m, 1H), 1.71 (dd, J=6.5, 4.5 Hz, 1H), 3.55-3.59 (m, 1H), 3.66-3.70 (m, 1H), 3.97-4.03 (m, 1H), 6.21 (d, J=10.0 Hz, 1H), 6.80 (t, J=8.0 Hz, 1H), 7.72 (dd, J=8.0, 1.5 Hz, 1H), 7.92 (dd, J=10, 2 Hz, 1H).
(2) Synthesis of (S)-iBu-NO₂: Pd(OAc)₂ (60 mg, 0.27 mmol, 3 mol %), SPhos (221 mg, 0.54 mmol, 6 mol %) and K₃PO₄ (3.77 g, 17.78 mmol, 2.0 equiv) were added to a dry tube equipped with a magnetic stir bar. The tube was evacuated and backfilled with nitrogen, the evacuation and back fill procedure was repeated for another twice. Then (S)-iPr-OH (2.85 g, 8.99 mmol, 1.0 equiv) and toluene (30 mL) were added under a nitrogen atmosphere. The tube was sealed and the mixture was stirred at a temperature of 100-110° C. for 1.5 days, cooled down to ambient temperature. The solvent was removed under reduced pressure and the residue was purified through column chromatography on silica gel using petroleum ether/ethyl acetate (30:1) as eluent to obtain the desired product (S)-iBu-NO₂ as a red oily liquid 1.85 g in 87% yield. ¹H NMR (500 MHz, CDCl₃): δ(ppm) 1.01 (dd, J=6.5, 4.0 Hz, 6H), 1.39-1.45 (m, 1H), 1.49-1.55 (m, 1H), 1.75-1.88 (m, 1H), 3.66-3.72 (m, 1H), 3.83 (dd, J=10.6, 6.9 Hz, 1H), 4.24 (ddd, J=10.6, 3.2, 1.1 Hz, 1H), 6.54 (dd, J=8.5, 7.5 Hz, 1H), 6.96-6.98 (m, 1H), 7.75 (dd, J=8.0, 1.5 Hz, 1H), 7.94 (s, 1H).
(3) Synthesis of (S)-iBu-NH₂: SnCl₂·2H₂O (7.22 g, 32.00 mmol, 4.0 equiv) was added to a three-necked flask equipped with a magnetic stir bar. The flask was evacuated and backfilled with nitrogen, the evacuation and back fill procedure was repeated for another twice. Then (S)-iBu-NO₂ (1.77 g, 8.00 mmol, 1.0 equiv), ethanol and ethyl acetate (1:1) were added under a nitrogen atmosphere. The flask was sealed and the mixture was stirred at a temperature of 78° C. for 16 hours, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/ethyl acetate (20:1-1:1) as eluent to obtain the desired product (S)-iBu-NH₂ as a yellow oily liquid 1.5 g in 90% yield. ¹H NMR (500 MHz, CDCl₃): δ(ppm) 0.98 (t, J=6.5 Hz, 6H), 1.30-1.36 (m, 1H), 1.39-1.45 (m, 1H), 1.79-1.87 (m, 1H), 2.79 (s, 3H), 3.38-3.43 (m, 1H), 3.72 (dd, J=10.5, 8.0 Hz, 1H), 4.19 (dd, J=10.5, 3.0 Hz, 1H), 6.34 (dd, J=7.5, 1.0 Hz, 1H), 6.39 (dd, J=8.0, 1.0 Hz, 1H), 6.62 (t, J=8.0 Hz, 1H).
(4) Synthesis of (S)-iBu-L6: 1-Cl (966 mg, 2.0 mmol, 1.0 equiv), Pd₂(dba)₃ (55 mg, 0.06 mmol, 3 mol %), JohnPhos (38 mg, 0.12 mmol, 6 mol %) and tBuONa (384 mg, 4.0 mmol, 2.0 equiv) were added to a dry tube equipped with a magnetic stir bar. The tube was evacuated and backfilled with nitrogen, the evacuation and back fill procedure was repeated for another twice. Then (S)-iBu-NH₂ (412 mg, 2.0 mmol, 1.0 equiv) and toluene (10 mL) were added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (30:1-20:1) as eluent to obtain the desired product (S)-iBu-L6 as a yellow foamy solid 1.23 g in 94% yield.
(5) Synthesis of (S)-iBu-LA6: (S)-iBu-L6 (1.23 g, 1.88 mmol, 1.0 equiv), NH₄PF₆ (614 mg, 3.77 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (8 mL) was added under a nitrogen atmosphere. The tube was sealed and the mixture was stirred at a temperature of 80° C. for 12 hours, cooled down to ambient temperature. The solvent was removed under reduced pressure and the residue was purified through column chromatography on silica gel using petroleum ether/ethyl acetate (10:1) first, then dichloromethane/ethyl acetate (20:1) as eluent to obtain the desired product (S)-iBu-LA6 as a yellow foamy solid 1.0 g in 66% yield. ¹H NMR (500 MHz, DMSO-d₆): δ(ppm) 0.96 (d, J=6.5 Hz, 3H), 1.00 (d, J=6.5 Hz, 3H), 1.30 (s, 9H), 1.36 (s, 9H), 1.72-1.77 (m, 1H), 1.79-1.87 (m, 1H), 1.94-1.98 (m, 1H), 4.49 (dd, J=12.0, 5.5 Hz, 1H), 4.67 (dd, J=12.0, 3.0 Hz, 1H), 4.89-4.94 (m, 1H), 7.19-7.22 (m, 1H), 7.25 (t, J=2.0 Hz, 1H), 7.32-7.36 (m, 2H), 7.44-7.49 (m, 3H), 7.50 (d, J=2.0 Hz, 1H), 7.52-7.56 (m, 2H), 7.68 (d, J=1.0 Hz, 1H), 7.74 (d, J=8.0 Hz, 1H), 8.24 (d, J=7.5 Hz, 1H), 8.32 (d, J=8.5 Hz, 1H), 8.58 (d, J=5.5 Hz, 1H), 10.19 (s, 1H).
(6) Synthesis of (S)-iBu-P—PtA6: (S)-iBu-LA6 (300 mg, 0.37 mmol, 1.0 equiv), Pt(COD)Cl₂ (146 mg, 0.39 mmol, 1.05 equiv) and NaOAc (91 mg, 1.11 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (22 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (S)-iBu-P—PtA6 as a pale yellow solid 205 mg in 65% yield. ¹H NMR (500 MHz, DMSO-d₆): δ(ppm) 0.37 (d, J=6.5 Hz, 3H), 0.58 (d, J=6.5 Hz, 3H), 0.78-0.89 (m, 1H), 1.27 (s, 9H), 1.45 (s, 9H), 1.60-1.66 (m, 1H), 1.79-1.91 (m, 1H), 4.52 (d, J=11.5 Hz, 1H), 4.72 (d, J=11.0 Hz, 1H), 4.89-4.95 (m, 1H), 6.94 (d, J=8.0 Hz, 1H), 7.01 (d, J=2.0 Hz, 1H), 7.23 (dd, J=6.0, 1.5 Hz, 1H), 7.29 (d, J=8.0 Hz, 1H), 7.38-7.45 (m, 3H), 7.49-7.53 (m, 1H), 7.66 (d, J=8.5 Hz, 1H), 7.90 (dd, J=8.5, 2.5 Hz, 2H), 8.08 (d, J=2.0 Hz, 1H), 8.16 (dd, J=7.5, 0.5 Hz 1H), 9.71 (d, J=6.5 Hz, 1H).

### Example 14

The synthetic route for (R)-2MeiBu-M-PtA7 is as follows:

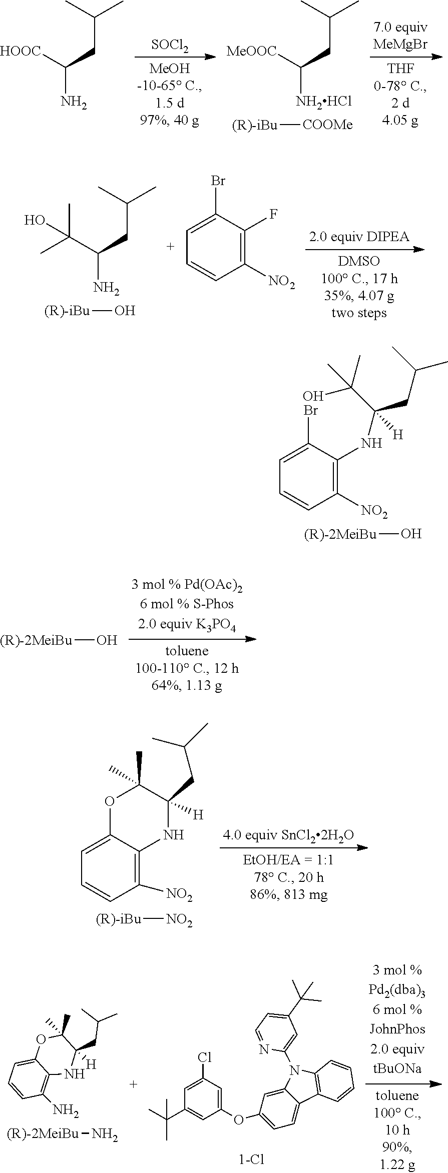

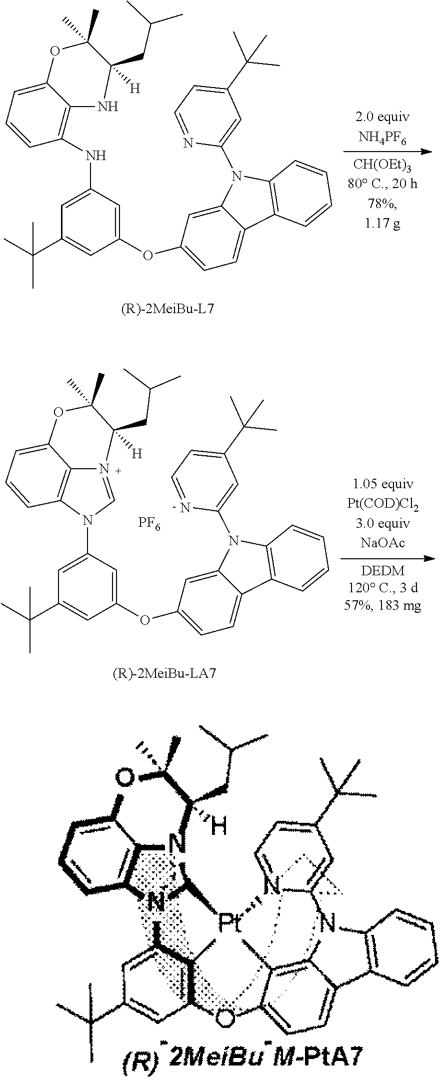

(1) Synthesis of (R)-iBu-COOMe: D-leucine (2 g, 9.09 mmol, 1.0 equiv) and methanol (160 mL) were added to a dry three-necked flask equipped with a magnetic stir bar. Subsequently, the mixture was stirred at −10° C. while SOCl₂ (48 mL) was slowly added and stirred for 30 minutes. Then the mixture was heated to 65° C. for 1.5 days, cooled down to ambient temperature. The solvent was removed under reduced pressure and the residue was purified through recrystallization using methanol/petroleum ether as solvent to obtain the desired product (R)-iBu-COOMe as a white solid 40 g in 97% yield.
(2) Synthesis of (R)-iBu-OH: MeMgBr (77 mL, 231 mmol, 7.0 equiv, 3.0 M in 2-MeTHF) and THE (75 mL) were added to a dry three-necked flask equipped with a magnetic stir bar. The flask was evacuated and backfilled with nitrogen, the evacuation and back fill procedure was repeated for another twice. The reaction mixture was cooled to 0° C., followed by the slow addition of (R)-iBu-COOMe (6 g, 33 mmol, 1.0 equiv), and the mixture was stirred for 10 minutes. Then the mixture was heated to 78° C. for 2 days, cooled down to ambient temperature. The pH of the reaction mixture was adjusted to around 7-8 using a saturated NH₄Cl solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure to obtain the crude product (R)-iBu-OH as a yellow oily liquid 4.05 g.
(3) Synthesis of (R)-2MeiBu-OH: 1-bromo-2-fluoro-3-nitrobenzene (4.04 g, 18.36 mmol, 1.0 equiv) was added to a dry tube equipped with a magnetic stir bar. The tube was evacuated and backfilled with nitrogen, the evacuation and back fill procedure was repeated for another twice. Then (R)-iBu-OH (4 g, 27.54 mmol, 1.5 equiv), N,N-Diisopropylethylamine (DIPEA) (4.75 g, 36.72 mmol, 2.0 equiv) and DMSO (20 mL) were added under a nitrogen atmosphere. The tube was sealed and the mixture was stirred at a temperature of 100° C. for 17 hours, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/dichloromethane (2:1) as eluent to obtain the desired product (R)-2MeiBu-OH as a red liquid 4.07 g in 25% yield (two steps). ¹H NMR (500 MHz, CDCl₃): δ(ppm) 0.84 (dd, J=8.5, 6.5 Hz, 6H), 1.19 (s, 3H), 1.28 (s, 3H), 1.30-1.37 (m, 1H), 1.37-1.43 (m, 1H), 1.46-1.50 (m, 1H), 1.98 (s, 1H), 4.15 (s, 1H), 6.55 (s, 1H), 6.72 (t, J=8.0 Hz, 1H), 7.69 (dd, J=7.5, 1.5 Hz, 1H), 7.93 (d, J=8.5 Hz, 1H).
(4) Synthesis of (R)-2MeiBu-NO₂: Pd(OAc)₂ (57 mg, 0.25 mmo 1, 3 mol %), SPhos (207 mg, 0.50 mmol, 6 mol %) and K₃PO₄ (3.56 g, 16.80 mmol, 2.0 equiv) were added to a dry tube equipped with a magnetic stir bar. The tube was evacuated and backfilled with nitrogen, the evacuation and back fill procedure was repeated for another twice. Then (R)-2MeiBu-OH (2.82 g, 8.89 mmol, 1.0 equiv) and toluene (30 mL) were added under a nitrogen atmosphere. The tube was sealed and the mixture was stirred at a temperature of 100-110° C. for 12 hours, cooled down to ambient temperature. The solvent was removed under reduced pressure and the residue was purified through column chromatography on silica gel using petroleum ether/ethyl acetate (20:1) as eluent to obtain the desired product (R)-2MeiBu-NO₂ as a red liquid 1.13 g in 64% yield. ¹H NMR (500 MHz, CDCl₃): δ(ppm) 0.99 (d, J=6.5 Hz, 3H), 1.04 (d, J=6.5z Hz, 3H), 1.17 (s, 3H), 1.36-1.41 (m, 4H), 1.82-1.91 (m, 1H), 6.55 (dd, J=9.0, 8.0 Hz, 1H), 6.94-6.95 (m, 1H), 7.73 (dd, J=9.0, 1.5 Hz, 1H), 7.97 (s, 1H).
(5) Synthesis of (R)-2MeiBu-NH₂: SnCl₂·2H₂O (3.65 g, 16.22 mmol, 4.0 equiv) was added to a three-necked flask equipped with a magnetic stir bar. The flask was evacuated and backfilled with nitrogen, the evacuation and back fill procedure was repeated for another twice. Then (R)-2MeiBu-NO₂ (1.07 g, 4.05 mmol, 1.0 equiv), ethanol and ethyl acetate (1:1) were added under a nitrogen atmosphere. The flask was sealed and the mixture was stirred at a temperature of 78° C. for 20 hours, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/ethyl acetate (20:1-10:1) as eluent to obtain the desired product (R)-2MeiBu-NH₂ as a light yellow liquid 813 mg in 86% yield. ¹H NMR (500 MHz, CDCl₃): δ(ppm) 0.95 (d, J=6.5 Hz, 3H), 0.99 (d, J=6.5 Hz, 3H), 1.12 (s, 3H), 1.23-1.39 (m, 5H), 1.83-1.96 (m, 1H), 3.00 (dd, J=10.5, 2.5 Hz, 1H), 3.26 (s, 2H), 6.32-6.37 (m, 1H), 6.63 (t, J=7.5 Hz, 1H).
(6) Synthesis of (R)-2MeiBu-L7: 1-Cl (966 mg, 2.0 mmol, 1.0 equiv), Pd₂(dba)₃ (55 mg, 0.06 mmol, 3 mol %), JohnPhos (38 mg, 0.12 mmol, 6 mol %) and tBuONa (344 mg, 4.0 mmol, 2.0 equiv) were added to a dry tube equipped with a magnetic stir bar. The tube was evacuated and backfilled with nitrogen, the evacuation and back fill procedure was repeated for another twice. Then (R)-2MeiBu-NH₂ (468 mg, 2.0 mmol, 1.0 equiv) and toluene (10 mL) were added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (30:1-20:1) as eluent to obtain the desired product (R)-2MeiBu-L7 as a pale yellow liquid 1.22 g in 90% yield.
(7) Synthesis of (R)-2MeiBu-A7: NH₄PF₆ (584 mg, 3.58 mmol, 2.0 equiv) was added to a dry tube equipped with a magnetic stir bar. The tube was evacuated and backfilled with nitrogen, the evacuation and back fill procedure was repeated for another twice. Then (R)-2MeiBu-L7 (1.22 g, 1.79 mmol, 1.0 equiv) and CH(OEt)₃ (8 mL) were added under a nitrogen atmosphere. The tube was sealed and the mixture was stirred at a temperature of 80° C. for 20 hours, cooled down to ambient temperature. The solvent was removed under reduced pressure and the residue was purified through column chromatography on silica gel using petroleum ether/ethyl acetate (10:1) first, then dichloromethane/ethyl acetate (20:1) as eluent to obtain the desired product (R)-2MeiBu-A7 as a yellow foamy solid 1.17 g in 78% yield. ¹H NMR (500 MHz, DMSO-d₆): δ(ppm) 0.87 (d, J=6.5 Hz, 3H), 1.02 (d, J=6.5 Hz, 3H), 1.30 (s, 9H), 1.36 (s, 9H), 1.37 (s, 3H), 1.49 (s, 3H), 1.53-1.62 (m, 1H), 1.61-1.69 (m, 1H), 1.73-1.78 (m, 1H), 4.72 (dd, J=9.5, 2.5 Hz, 1H), 7.13 (d, J=8.0 Hz, 1H), 7.20 (dd, J=8.5, 2.5 Hz, 1H), 7.27 (d, J=8.0 Hz, 1H), 7.31-7.37 (m, 2H), 7.43-7.49 (m, 3H), 7.50 (d, J=2.5 Hz 1H), 7.53 (t, J=8.5 Hz, 1H), 7.61 (t, J=1.5 Hz, 1H), 7.67 (d, J=1.0 Hz, 1H), 7.74 (d, J=8.5 Hz 1H), 8.24 (d, J=7.5 Hz, 1H), 8.32 (d, J=8.5 Hz, 1H), 8.58 (d, J=5.5 Hz, 1H), 10.11 (s, 1H).
(8) Synthesis of (R)-2MeiBu-M-PtA7: (R)-2MeiBu-A7 (300 mg, 0.36 mmol, 1.0 equiv), Pt(COD)Cl₂ (141 mg, 0.38 mmol, 1.05 equiv) and NaOAc (88 mg, 1.11 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (22 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (4:1) as eluent to obtain the desired product (R)-2MeiBu-M-PtA7 as a pale yellow solid 183 mg in 57% yield. ¹H NMR (500 MHz, DMSO-d₆): δ(ppm) 0.39 (d, J=6.5 Hz, 3H), 0.42 (d, J=6.5 Hz, 3H), 0.99-1.08 (m, 1H), 1.37 (s, 9H), 1.45 (s, 9H), 1.59 (s, 3H), 1.60 (s, 3H), 1.64-1.72 (m, 1H), 2.08-2.16 (m, 1H), 4.53 (dd, J=10.0, 2.5 Hz, 1H), 6.93 (d, J=8.0 Hz, 1H), 7.00 (d, J=1.5 Hz, 1H), 7.29 (d, J=8.5 Hz, 1H), 7.39-7.44 (m, 2H), 7.47-7.57 (m, 3H), 7.77 (d, J=8.3 Hz, 1H), 7.90 (d, J=8.5 Hz, 1H), 7.97 (d, J=8.0 Hz, 1H), 8.15 (d, J=2.0 Hz, 1H), 8.16-8.18 (m, 1H), 9.39 (d, J=6.0 Hz, 1H).

### Example 15

The synthetic route for (S)-2MeiBu-P—PtA7 is as follows:

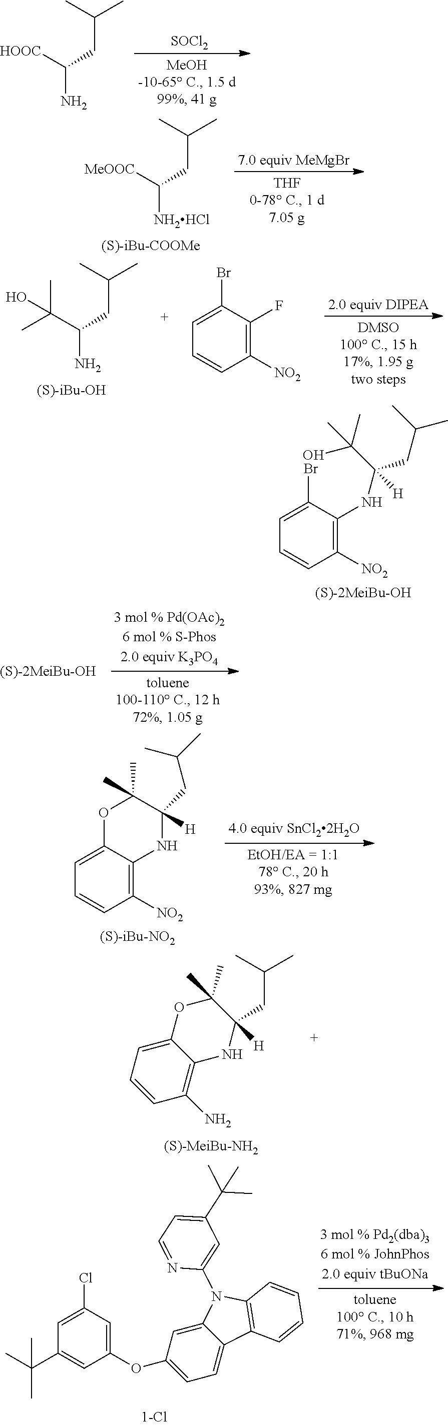

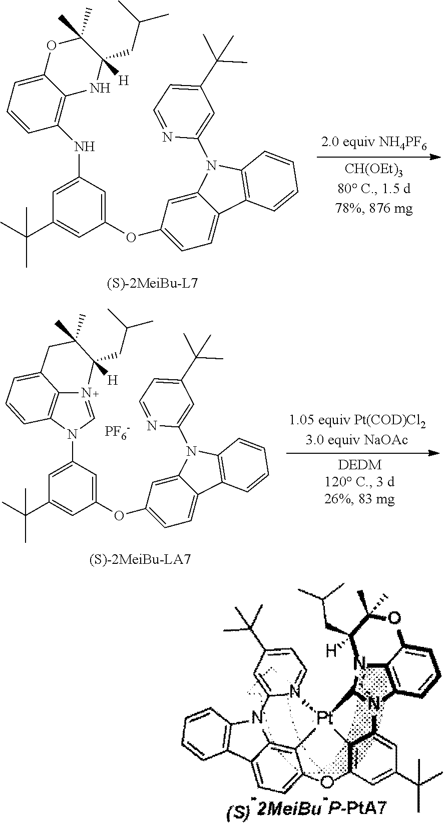

(1) Synthesis of (S)-iBu-COOMe: L-leucine (30 g, 22.087 mmol, 1.0 equiv) and methanol (160 mL) were added to a dry three-necked flask equipped with a magnetic stir bar. Subsequently, the mixture was stirred at −10° C. while SOCl₂ (48 mL) was slowly added and stirred for 30 minutes. Then the mixture was heated to 65° C. for 1.5 days, cooled down to ambient temperature. The solvent was removed under reduced pressure and the residue was purified through recrystallization using methanol/petroleum ether as solvent to obtain the desired product (S)-iBu-COOMe as a white solid 41 g in 99% yield.
(2) Synthesis of (S)-iBu-OH: MeMgBr (77 mL, 231 mmol, 7.0 equiv, 3.0 M in 2-MeTHF) and THE (75 mL) were added to a dry three-necked flask equipped with a magnetic stir bar. The flask was evacuated and backfilled with nitrogen, the evacuation and back fill procedure was repeated for another twice. The reaction mixture was cooled to 0° C., followed by the slow addition of (S)-iBu-COOMe (6 g, 33 mmol, 1.0 equiv), and the mixture was stirred for 10 minutes. Then the mixture was heated to 78° C. for 1 days, cooled down to ambient temperature. The pH of the reaction mixture was adjusted to around 7-8 using a saturated NH₄Cl solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure to obtain the crude product (S)-iBu-OH as a yellow oily liquid 7.05 g.
(3) Synthesis of (S)-2MeiBu-OH: 1-bromo-2-fluoro-3-nitrobenzene (7.07 g, 32.13 mmol, 1.0 equiv) was added to a dry tube equipped with a magnetic stir bar. The tube was evacuated and backfilled with nitrogen, the evacuation and back fill procedure was repeated for another twice. Then (S)-iBu-OH (7 g, 48.19 mmol, 1.5 equiv), N,N-Diisopropylethylamine (DIPEA) (8.3 g, 64.25 mmol, 2.0 equiv) and DMSO (35 mL) were added under a nitrogen atmosphere. The tube was sealed and the mixture was stirred at a temperature of 100° C. for 15 hours, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/dichloromethane (2:1) as eluent to obtain the desired product (S)-2MeiBu-OH as a red liquid 1.95 g in 17% yield (two steps). ¹H NMR (500 MHz, CDCl₃): δ(ppm) 0.84 (dd, J=8.5, 6.5 Hz, 6H), 1.19 (s, 3H), 1.28 (s, 3H), 1.30-1.35 (m, 1H), 1.37-1.43 (m, 1H), 1.46-1.50 (m, 1H), 1.98 (s, 1H), 4.15 (s, 1H), 6.56 (s, 1H), 6.72 (t, J=8.0 Hz, 1H), 7.69 (dd, J=8.0, 2.0 Hz, 1H), 7.93 (d, J=8.5 Hz, 1H).
(4) Synthesis of (S)-2MeiBu-NO₂: Pd(OAc)₂ (37 mg, 0.17 mmo 1, 3 mol %), SPhos (136 mg, 0.33 mmol, 6 mol %) and K₃PO₄ (2.34 g, 11.01 mmol, 2.0 equiv) were added to a dry tube equipped with a magnetic stir bar. The tube was evacuated and backfilled with nitrogen, the evacuation and back fill procedure was repeated for another twice. Then (S)-2MeiBu-OH (2.82 g, 8.89 mmol, 1.0 equiv) and toluene (15 mL) were added under a nitrogen atmosphere. The tube was sealed and the mixture was stirred at a temperature of 100-110° C. for 12 hours, cooled down to ambient temperature. The solvent was removed under reduced pressure and the residue was purified through column chromatography on silica gel using petroleum ether/ethyl acetate (50:1) as eluent to obtain the desired product (S)-2MeiBu-NO₂ as a red liquid 1.05 g in 72% yield. ¹H NMR (500 MHz, CDCl₃): δ(ppm) 0.99 (d, J=6.5 Hz, 3H), 1.04 (d, J=6.5 Hz, 3H), 1.17 (s, 3H), 1.35-1.42 (m, 4H), 1.81-1.92 (m, 1H), 3.32-3.35 (m, 1H), 6.55 (dd, J=9.0, 8.0 Hz, 1H), 6.94 (d, J=8.0 Hz, 1H), 7.73 (dd, J=8.5, 1.5 Hz, 1H), 7.97 (s, 1H),
(5) Synthesis of (S)-2MeiBu-NH₂: SnCl₂·2H₂O (3.42 g, 15.14 mmol, 4.0 equiv) was added to a three-necked flask equipped with a magnetic stir bar. The flask was evacuated and backfilled with nitrogen, the evacuation and back fill procedure was repeated for another twice. Then (S)-2MeiBu-NO₂ (1.00 g, 3.79 mmol, 1.0 equiv), ethanol and ethyl acetate (1:1) were added under a nitrogen atmosphere. The flask was sealed and the mixture was stirred at a temperature of 78° C. for 20 hours, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/ethyl acetate (20:1-10:1) as eluent to obtain the desired product (S)-2MeiBu-NH₂ as a light yellow liquid 827 mg in 93% yield. ¹H NMR (500 MHz, CDCl₃): δ(ppm) 0.94 (d, J=6.5 Hz, 3H), 0.98 (d, J=6.5 Hz, 3H), 1.12 (s, 3H), 1.22-1.39 (m, 5H), 1.60 (s, 1H), 1.85-1.93 (m, 1H), 3.00 (dd, J=10.5, 3.0 Hz, 1H), 3.36 (s, 1H), 6.29-6.46 (m, 2H), 6.63 (t, J=8.0. Hz, 1H).
(6) Synthesis of (S)-2MeiBu-L7: 1-Cl (966 mg, 2.0 mmol, 1.0 equiv), Pd₂(dba)₃ (55 mg, 0.06 mmol, 3 mol %), JohnPhos (38 mg, 0.12 mmol, 6 mol %) and tBuONa (344 mg, 4.0 mmol, 2.0 equiv) were added to a dry tube equipped with a magnetic stir bar. The tube was evacuated and backfilled with nitrogen, the evacuation and back fill procedure was repeated for another twice. Then (S)-2MeiBu-NH₂ (468 mg, 2.0 mmol, 1.0 equiv) and toluene (10 mL) were added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (30:1-20:1) as eluent to obtain the desired product (S)-2MeiBu-L7 as a pale yellow liquid 968 mg in 71% yield.
(7) Synthesis of (S)-2MeiBu-A7: NH₄PF₆ (463 mg, 2.84 mmol, 2.0 equiv) was added to a dry tube equipped with a magnetic stir bar. The tube was evacuated and backfilled with nitrogen, the evacuation and back fill procedure was repeated for another twice. Then (S)-2MeiBu-L7 (968 mg, 1.42 mmol, 1.0 equiv) and CH(OEt)₃ (8 mL) were added under a nitrogen atmosphere. The tube was sealed and the mixture was stirred at a temperature of 80° C. for 1.5 days, cooled down to ambient temperature. The solvent was removed under reduced pressure and the residue was purified through column chromatography on silica gel using petroleum ether/ethyl acetate (10:1) first, then dichloromethane/ethyl acetate (20:1) as eluent to obtain the desired product (S)-2MeiBu-A7 as a yellow foamy solid 876 mg in 78% yield. ¹H NMR (500 MHz, DMSO-d₆): δ(ppm) 0.87 (d, J=6.0 Hz, 3H), 1.02 (d, J=6.5 Hz, 3H), 1.30 (s, 9H), 1.36 (s, 9H), 1.37 (s, 3H), 1.49 (s, 3H), 1.54-1.62 (m, 1H), 1.63-1.69 (m, 1H), 1.73-1.78 (m, 1H), 4.72 (dd, J=10.0, 2.5 Hz, 1H), 7.13 (d, J=8.0 Hz, 1H), 7.20 (dd, J=8.5, 2.0 Hz, 1H), 7.28 (d, J=8.5 Hz, 2H), 7.32-7.38 (m, 2H), 7.43-7.49 (m, 3H), 7.50 (d, J=2.5 Hz, 1H), 7.53 (t, J=8.0 Hz, 1H), 7.61 (t, J=1.5 Hz, 1H), 7.67 (d, J=1.0 Hz, 1H), 7.74 (d, J=8.0 Hz, 1H), 8.24 (d, J=7.5 Hz, 1H), 8.32 (d, J=8.5 Hz, 1H), 8.58 (d, J=5.5 Hz, 1H), 10.12 (s, 1H).
(8) Synthesis of (S)-2MeiBu-P—PtA7: (S)-2MeiBu-A7 (300 mg, 0.36 mmol, 1.0 equiv), Pt(COD)Cl₂ (141 mg, 0.38 mmol, 1.05 equiv) and NaOAc (88 mg, 1.11 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (22 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (4:1) as eluent to obtain the desired product (S)-2MeiBu-P—PtA7 as a pale yellow solid 83 mg in 26% yield. ¹H NMR (500 MHz, DMSO-d₆): δ(ppm) 0.39 (d, J=6.5 Hz, 3H), 0.42 (d, J=6.5 Hz, 3H), 1.00-1.06 (m, 1H), 1.37 (s, 9H), 1.45 (s, 9H), 1.58 (s, 3H), 1.60 (s, 3H), 1.64-1.72 (m, 1H), 2.10-2.16 (m, 1H), 4.53 (dd, J=9.5, 2.0 Hz, 1H), 6.93 (d, J=8.0 Hz, 1H), 7.00 (d, J=1.5 Hz, 1H), 7.29 (d, J=8.5 Hz, 1H), 7.39-7.44 (m, 2H), 7.48-7.61 (m, 3H), 7.77 (d, J=8.5 Hz, 1H), 7.90 (d, J=8.0 Hz, 1H), 7.97 (d, J=8.1 Hz, 1H), 8.14 (d, J=2.0 Hz, 1H), 8.17 (d, J=8.0 Hz, 1H), 9.39 (d, J=6.5 Hz, 1H).

### Example 16

The synthetic route for (R)-Bn-M-PtA8 is as follows:

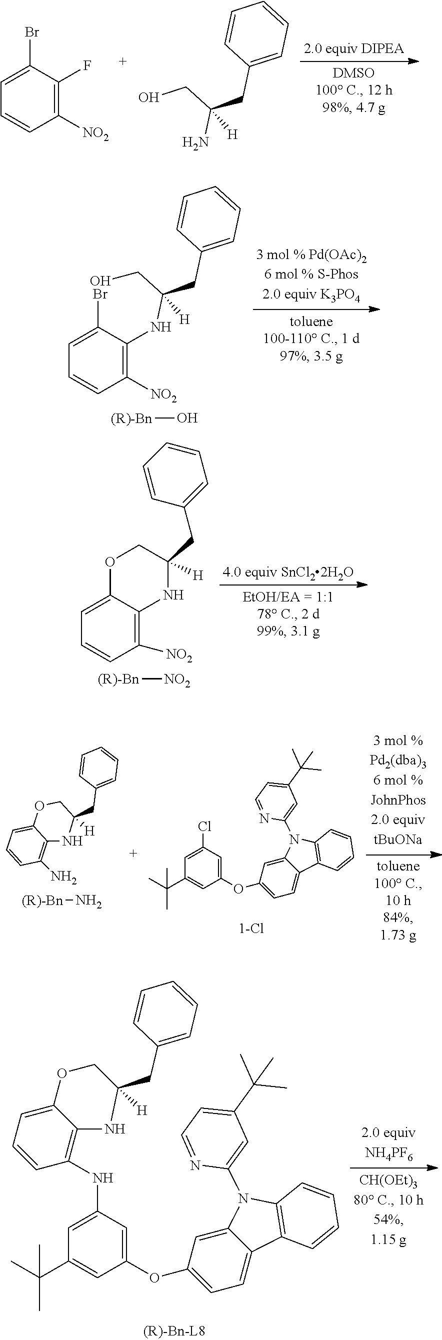

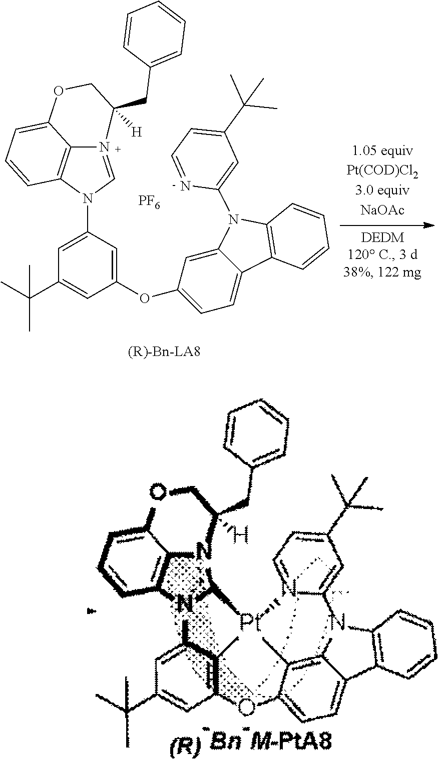

(1) Synthesis of (R)-Bn-OH: 1-bromo-2-fluoro-3-nitrobenzene (3.0 g, 13.64 mmol, 1.0 equiv), (R)-2-amino-3-phenylpropan-1-ol (2.06 g, 13.64 mmol, 1.0 equiv) were added to a dry tube equipped with a magnetic stir bar. The tube was evacuated and backfilled with nitrogen, the evacuation and back fill procedure was repeated for another twice. Then N,N-Diisopropylethylamine (DIPEA) (3.53 g, 27.28 mmol, 2.0 equiv) and DMSO (20 mL) were added under a nitrogen atmosphere. The tube was sealed and the mixture was stirred at a temperature of 100° C. for 12 hours, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate (15:1-5:1) as eluent to obtain the desired product (R)-Bn-OH as a yellow oily liquid 4.7 g in 98% yield. ¹H NMR (500 MHz, CDCl₃): δ(ppm) 1.76 (dd, J=6.0, 5.0 Hz, 1H), 2.90 (d, J=7.5 Hz, 2H), 3.58-3.62 (m, 1H), 3.67-3.71 (m, 1H), 4.17-4.24 (m, 1H), 6.52 (d, J=10.5 Hz, 1H), 6.74 (dd, J=8.5, 8.0 Hz, 1H), 7.13-7.16 (m, 3H), 7.20-7.23 (m, 2H), 7.68 (dd, J=8.0, 1.5 Hz, 1H), 7.86 (dd, J=8.5, 2.0 Hz, 1H).
(2) Synthesis of (R)-Bn-NO₂: Pd(OAc)₂ (90 mg, 0.40 mmol, 3 mol %), SPhos (328 mg, 0.80 mmol, 6 mol %) and K₃PO₄ (5.68 g, 26.76 mmol, 2.0 equiv) were added to a dry tube equipped with a magnetic stir bar. The tube was evacuated and backfilled with nitrogen, the evacuation and back fill procedure was repeated for another twice. Then (R)-Bn-OH (4.7 g, 13.38 mmol, 1.0 equiv) and toluene (50 mL) were added under a nitrogen atmosphere. The tube was sealed and the mixture was stirred at a temperature of 100-110° C. for 1 days, cooled down to ambient temperature. The solvent was removed under reduced pressure and the residue was purified through column chromatography on silica gel using petroleum ether/ethyl acetate (10:1) as eluent to obtain the desired product (R)-Bn-NO₂ as a yellow oily liquid 3.5 g in 97% yield. ¹H NMR (500 MHz, CDCl₃): δ(ppm) 2.84 (dd, J=13.5, 8.0 Hz, 1H), 2.95 (dd, J=13.0, 6.0 Hz, 1H), 3.80-3.86 (m, 1H), 3.95 (dd, J=10.5, 6.0 Hz, 1H), 4.22-4.25 (m, 1H), 6.55 (dd, J=9.0, 8.0 Hz, 1H), 6.99-7.01 (m, 1H), 7.25 (s, 1H), 7.26-7.27 (m, 1H), 7.29-7.32 (m, 1H), 7.37-7.40 (m, 2H), 7.76 (dd, J=8.2, 1.5 Hz, 1H), 7.90 (s, 1H).
(3) Synthesis of (R)-Bn-NH₂: (R)-Bn-NO₂ (3.50 g, 12.95 mmol, 1.0 equiv), SnCl₂·2H₂O (11.69 g, 51.80 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (20 mL:20 mL) were added under a nitrogen atmosphere. The flask was sealed and the mixture was stirred at a temperature of 78° C. for 2 days, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/ethyl acetate/dichloromethane (10:1-5:1:1) as eluent to obtain the desired product (R)-Bn-NH₂ as a Brown yellow solid 3.1 g in 99% yield. ¹H NMR (500 MHz, CDCl₃): δ(ppm) 2.79-3.03 (m, 2H), 3.03 (s, 3H), 3.59-3.64 (m, 1H), 3.91 (dd, J=10.5, 7.0 Hz, 1H), 4.21 (dd, J=10.5, 2.5 Hz, 1H), 6.34 (dd, J=8.0, 1.5 Hz, 1H), 6.41 (dd, J=8.5, 1.5 Hz, 1H), 6.60 (t, J=8.0 Hz, 1H), 7.23-7.25 (m, 2H), 7.26-7.28 (m, 1H), 7.33-7.36 (m, 2H).
(4) Synthesis of (R)-Bn-L8: (R)-Bn-NH₂ (721 mg, 3.0 mmol, 1.0 equiv), 1-Cl (1.45 g, 3.0 mmol, 1.0 equiv), Pd₂(dba)₃ (43 mg, 0.09 mmol, 3 mol %), JohnPhos (54 mg, 0.18 mmol, 6 mol %) and tBuONa (577 mg, 6.0 mmol, 2.0 equiv) were added to a dry tube equipped with a magnetic stir bar. The tube was evacuated and backfilled with nitrogen, the evacuation and back fill procedure was repeated for another twice. Then toluene (15 mL) was added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (30:1) as eluent to obtain the desired product (R)-Bn-L8 as a yellow foamy solid 1.73 g in 84% yield. ¹H NMR (500 MHz, DMSO-d₆): δ(ppm) 1.21 (s, 9H), 1.29 (s, 9H), 2.62-2.72 (m, 2H), 3.42-3.45 (m, 1H), 3.68 (dd, J=10.5, 6.5 Hz, 1H), 3.95 (dd, J=10.0, 2.0 Hz, 1H), 4.66 (s, 1H), 6.15 (t, J=1.5 Hz, 1H), 6.38 (t, J=8.0 Hz, 1H), 6.42 (dd, J=8.0, 1.5 Hz, 1H), 6.49 (t, J=2.0 Hz, 1H), 6.57 (dd, J=7.5, 1.5 Hz, 1H), 6.59 (t, J=1.5 Hz, 1H), 7.03 (dd, J=8.5, 2.0 Hz, 1H), 7.09-7.12 (m, 2H), 7.14-7.18 (m, 1H), 7.19-7.23 (m, 2H), 7.27 (s, 1H), 7.30-7.34 (m, 1H), 7.34 (d, J=2.0 Hz, 1H), 7.41-7.44 (m, 1H), 7.45 (dd, J=5.0, 1.5 Hz, 1H), 7.60 (d, J=1.5 Hz, 1H), 7.72 (d, J=8.5 Hz, 1H), 8.19 (d, J=7.5 Hz, 1H), 8.21 (d, J=8.5 Hz, 1H), 8.56 (dd, J=5.5, 0.5 Hz, 1H).
(5) Synthesis of (R)-Bn-LA8: (R)-Bn-L8 (1.73 g, 2.52 mmol, 1.0 equiv), NH₄PF₆ (822 mg, 5.04 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (6 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (10:1) first, then dichloromethane/ethyl acetate (40:1) as eluent to obtain the desired product (R)-Bn-LA8 as a yellow foamy solid 1.15 g in 54% yield. ¹H NMR (500 MHz, DMSO-d₆): δ(ppm) 1.30 (s, 9H), 1.35 (s, 9H), 3.21 (dd, J=14.0, 8.5 Hz, 1H), 3.36-3.40 (m, 1H), 4.48 (dd, J=12.0, 3.0 Hz, 1H), 4.55 (dd, J=12.0, 4.0 Hz, 1H), 5.16-5.21 (m, 1H), 7.16 (t, J=2.0 Hz, 1H), 7.19-7.24 (m, 5H), 7.29-7.37 (m, 4H), 7.45-7.49 (m, 4H), 7.52 (d, J=2.0 Hz, 1H), 7.56 (t, J=8.0 Hz, 1H), 7.68 (d, J=1.0 Hz, 1H), 7.74 (d, J=8.5 Hz, 1H), 8.25 (d, J=7.5 Hz, 1H), 8.34 (d, J=8.5 Hz, 1H), 8.58 (d, J=5.5 Hz, 1H), 9.89 (s, 1H).
(6) Synthesis of (R)-Bn-M-PtA8: (R)-Bn-LA8 (300 mg, 0.36 mmol, 1.0 equiv), Pt(COD)Cl₂ (142 mg, 0.38 mmol, 1.05 equiv) and NaOAc (89 mg, 1.08 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (22 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (R)-Bn-M-PtA8 as a pale yellow solid 122 mg in 38% yield. ¹H NMR (500 MHz, CDCl₃): δ(ppm) 0.93 (s, 9H), 1.55 (s, 9H), 2.78 (t, J=11 Hz, 1H), 3.21 (d, J=12.0 Hz, 1H), 3.55 (s, 1H), 4.19 (d, J=11.5 Hz, 1H), 4.84 (d, J=11.5 Hz, 1H), 6.59 (s, 3H), 6.87 (d, J=8.0 Hz, 1H), 7.00-7.02 (m, 1H), 7.08-7.14 (m, 3H), 7.17 (d, J=1.5 Hz, 1H), 7.26-7.36 (m, 3H), 7.44 (d, J=8.0 Hz, 1H), 7.75 (d, J=8.5 Hz, 1H), 7.82 (d, J=8.5 Hz, 1H), 7.98 (s, 1H), 7.98-8.02 (m, 1H), 10.24 (s, 1H).

### Example 17

The synthetic route for (S)-Bn-P—PtA8 is as follows:

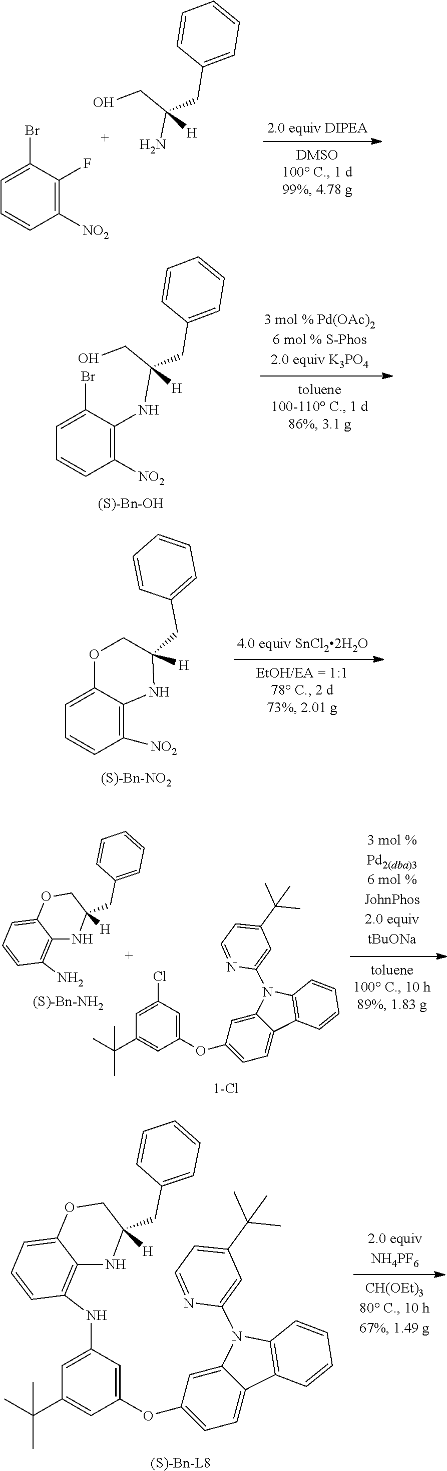

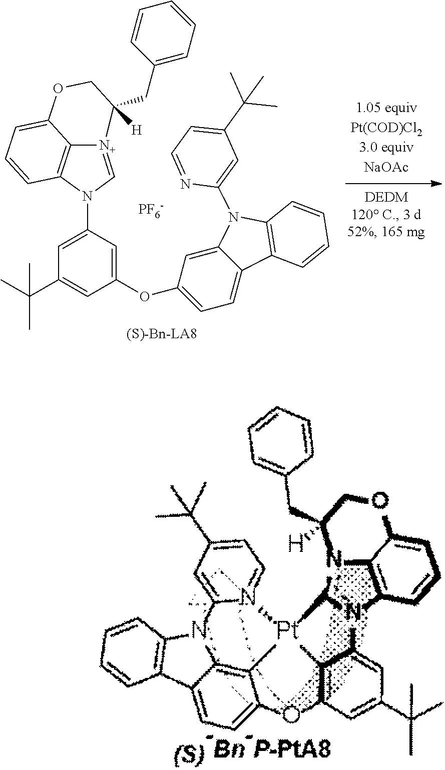

(1) Synthesis of (S)-Bn-OH: 1-bromo-2-fluoro-3-nitrobenzene (3.0 g, 13.64 mmol, 1.0 equiv), (S)-2-amino-3-phenylpropan-1-ol (2.06 g, 13.64 mmol, 1.0 equiv) were added to a dry tube equipped with a magnetic stir bar. The tube was evacuated and backfilled with nitrogen, the evacuation and back fill procedure was repeated for another twice. Then N,N-Diisopropylethylamine (DIPEA) (3.53 g, 27.28 mmol, 2.0 equiv) and DMSO (20 mL) were added under a nitrogen atmosphere. The tube was sealed and the mixture was stirred at a temperature of 100° C. for 1 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate (15:1-5:1) as eluent to obtain the desired product (S)-Bn-OH as a yellow oily liquid 4.78 g in 99% yield. ¹H NMR (500 MHz, CDCl₃): δ(ppm) 1.81 (dd, J=6.5, 4.5 Hz, 1H), 2.90 (d, J=7.5 Hz, 2H), 3.57-3.61 (m, 1H), 3.67-3.71 (m, 1H), 4.17-4.24 (m, 1H), 6.52 (d, J=10.5 Hz, 1H), 6.74 (t, J=8.0 Hz, 1H), 7.13-7.16 (m, 3H), 7.18-7.24 (m, 2H), 7.68 (dd, J=8.0, 2.0 Hz, 1H), 7.86 (dd, J=8.0, 1.5 Hz, 1H).
(2) Synthesis of (S)-Bn-NO₂: Pd(OAc)₂ (90 mg, 0.40 mmol, 3 mol %), SPhos (328 mg, 0.80 mmol, 6 mol %) and K₃PO₄ (5.68 g, 26.76 mmol, 2.0 equiv) were added to a dry tube equipped with a magnetic stir bar. The tube was evacuated and backfilled with nitrogen, the evacuation and back fill procedure was repeated for another twice. Then (S)-Bn-OH (4.78 g, 13.38 mmol, 1.0 equiv) and toluene (40 mL) were added under a nitrogen atmosphere. The tube was sealed and the mixture was stirred at a temperature of 100-110° C. for 1 days, cooled down to ambient temperature. The solvent was removed under reduced pressure and the residue was purified through column chromatography on silica gel using petroleum ether/ethyl acetate (10:1) as eluent to obtain the desired product (S)-Bn-NO₂ as a yellow oily liquid 3.1 g in 86% yield. ¹H NMR (500 MHz, CDCl₃): δ(ppm) 2.84 (dd, J=13.5, 8.5 Hz, 1H), 2.95 (dd, J=13.5, 6.5 Hz, 1H), 3.81-3.86 (m, 1H), 3.95 (dd, J=10.5, 6.0 Hz, 1H), 4.22-4.25 (m, 1H), 6.55 (dd, J=8.5, 7.5 Hz, 1H), 6.99-7.01 (m, 1H), 7.25 (s, 1H), 7.27 (d, J=1.5 Hz, 1H), 7.29-7.32 (m, 1H), 7.37-7.40 (m, 2H), 7.76 (dd, J=8.5, 1.5 Hz, 1H), 7.90 (s, 1H).
(3) Synthesis of (S)-Bn-NH₂: (S)-Bn-NO₂ (3.1 g, 11.47 mmol, 1.0 equiv), SnCl₂·2H₂O (10.53 g, 45.88 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (20 mL:20 mL) were added under a nitrogen atmosphere. The flask was sealed and the mixture was stirred at a temperature of 78° C. for 2 days, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/ethyl acetate/dichloromethane (10:1-5:1:1) as eluent to obtain the desired product (S)-Bn-NH₂ as a Brown yellow solid 2.01 g in 73% yield. ¹H NMR (500 MHz, CDCl₃): δ(ppm) 2.79-2.88 (m, 1H), 3.10 (s, 3H), 3.59-3.64 (m, 1H), 3.91 (dd, J=11.0, 730 Hz, 1H), 4.21 (dd, J=10.5, 3.0 Hz, 1H), 6.34 (dd, J=7.5, 1.0 Hz, 1H), 6.41 (dd, J=8.5, 1.5 Hz, 1H), 6.60 (t, J=7.5 Hz, 1H), 7.23-7.25 (m, 2H), 7.27 (dt, J=7.5, 2.5 Hz, 1H), 7.32-7.36 (m, 2H).
(4) Synthesis of (S)-Bn-L8: (S)-Bn-NH₂ (721 mg, 3.0 mmol, 1.0 equiv), 1-Cl (1.45 g, 3.0 mmol, 1.0 equiv), Pd₂(dba)₃ (43 mg, 0.09 mmol, 3 mol %), JohnPhos (54 mg, 0.18 mmol, 6 mol %) and tBuONa (577 mg, 6.0 mmol, 2.0 equiv) were added to a dry tube equipped with a magnetic stir bar. The tube was evacuated and backfilled with nitrogen, the evacuation and back fill procedure was repeated for another twice. Then toluene (15 mL) was added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (30:1) as eluent to obtain the desired product (S)-Bn-L8 as a yellow foamy solid 1.83 g in 89% yield. ¹H NMR (500 MHz, DMSO-d₆): δ(ppm) 1.21 (s, 9H), 1.29 (s, 9H), 2.62-2.72 (m, 2H), 3.43-3.46 (m, 1H), 3.68 (dd, J=11.0, 6.5 Hz, 1H), 3.96 (dd, J=10.5, 2.0 Hz, 1H), 4.66 (s, 1H), 6.16 (t, J=2.0 Hz, 1H), 6.38 (t, J=7.5 Hz, 1H), 6.42 (dd, J=8.0, 1.5 Hz, 1H), 6.49 (t, J=1.5 Hz, 1H), 6.57 (dd, J=7.5, 1.5 Hz, 1H), 6.59 (t, J=1.5 Hz, 1H), 7.04 (dd, J=8.0, 2.0 Hz, 1H), 7.09-7.11 (m, 2H), 7.14-7.18 (m, 1H), 7.20-7.23 (m, 2H), 7.27 (s, 1H), 7.30-7.33 (m, 1H), 7.34 (d, J=2.0 Hz, 1H), 7.41-7.44 (m, 1H), 7.45 (dd, J=5.5, 2.0 Hz, 1H), 7.60 (d, J=1.5 Hz, 1H), 7.72 (d, J=8.0 Hz, 1H), 8.19 (d, J=7.5 Hz, 1H), 8.21 (d, J=8.5 Hz, 1H), 8.56 (dd, J=5.5, 0.5 Hz, 1H).
(5) Synthesis of (S)-Bn-LA8: (S)-Bn-L8 (1.83 g, 2.66 mmol, 1.0 equiv), NH₄PF₆ (867 mg, 5.32 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (10 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (5:1) first, then dichloromethane/ethyl acetate (40:1) as eluent to obtain the desired product (S)-Bn-LA8 as a yellow foamy solid 1.49 g in 67% yield. ¹H NMR (500 MHz, DMSO-d₆): δ(ppm) 1.30 (s, 9H), 1.35 (s, 9H), 3.21 (dd, J=14.0, 8.5 Hz, 1H), 3.36-3.40 (m, 1H), 4.48 (dd, J=12.0, 3.0 Hz, 1H), 4.55 (dd, J=12.0, 4.0 Hz, 1H), 5.16-5.21 (m, 1H), 7.16 (t, J=2.0 Hz, 1H), 7.19-7.24 (m, 5H), 7.29-7.32 (m, 2H), 7.33-7.37 (m, 2H), 7.45-7.49 (m, 4H), 7.52 (d, J=2.0 Hz, 1H), 7.56 (t, J=8.0 Hz, 1H), 7.68 (d, J=1.0 Hz, 1H), 7.74 (d, J=8.5 Hz, 1H), 8.25 (d, J=7.5 Hz, 1H), 8.34 (d, J=8.5 Hz, 1H), 8.58 (dd, J=5.5, 0.5 Hz, 1H), 9.89 (s, 1H).
(6) Synthesis of (S)-Bn-P—PtA8: (S)-Bn-LA8 (300 mg, 0.36 mmol, 1.0 equiv), Pt(COD)Cl₂ (142 mg, 0.38 mmol, 1.05 equiv) and NaOAc (89 mg, 1.08 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (22 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (S)-Bn-P—PtA8 as a pale yellow solid 165 mg in 52% yield. ¹H NMR (500 MHz, CDCl₃): δ(ppm) 0.81 (s, 9H), 1.48 (s, 9H), 2.74 (t, J=13.0 Hz, 1H), 3.16 (d, J=13 Hz, 1H), 3.33 (s, 1H), 4.12 (d, J=11.5 Hz, 1H), 4.79 (d, J=11.5 Hz, 1H), 6.49-6.55 (m, 3H), 6.87 (d, J=8.0 Hz, 2H), 7.05-7.13 (m, 4H), 7.17 (d, J=1.5 Hz, 1H), 7.28-7.34 (m, 3H), 7.44 (d, J=8.5 Hz, 1H), 7.72 (d, J=8.0 Hz, 2H), 7.82 (d, J=8.5 Hz, 1H), 7.88 (s, 1H), 7.99-8.01 (m, 1H), 10.40 (s, 1H).

### Example 18

The synthetic route for (R)-2MeBn-M-PtA9 is as follows:

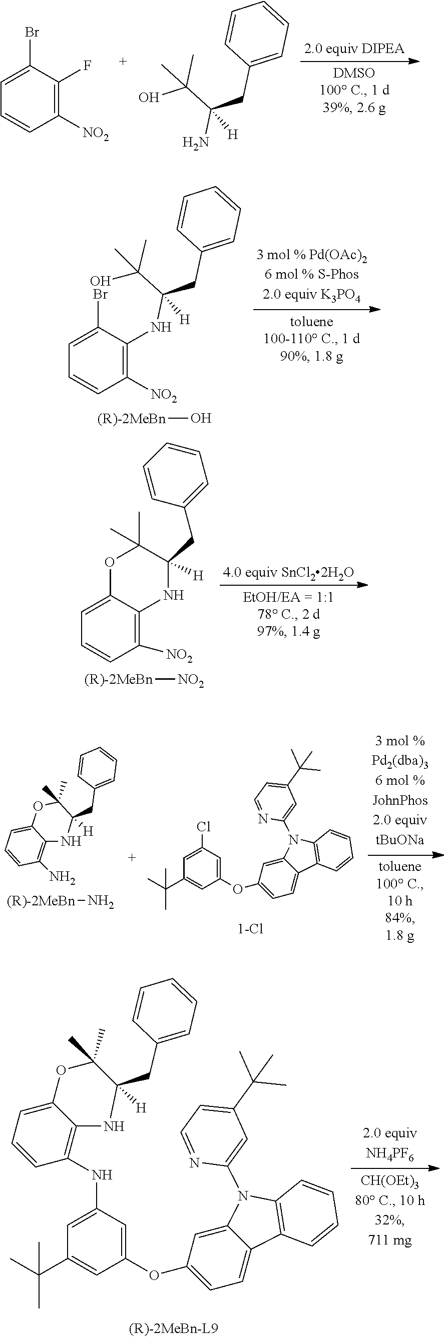

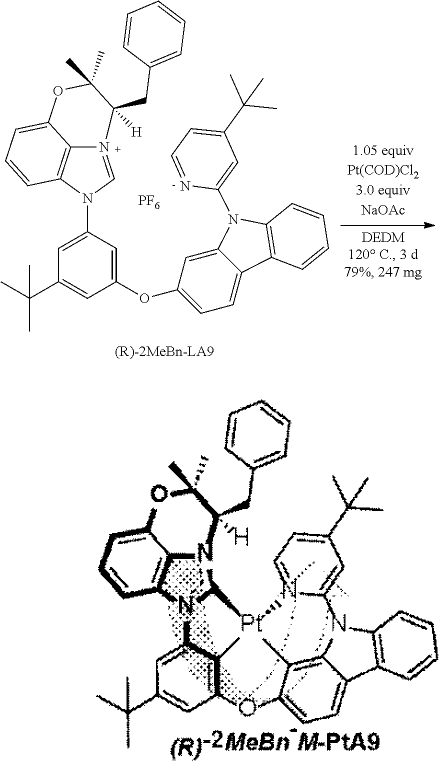

(1) Synthesis of (R)-2MeBn-OH: 1-bromo-2-fluoro-3-nitrobenzene (3.82 g, 17.38 mmol, 1.0 equiv), (R)-3-amino-2-methyl-4-phenylbutan-2-ol (3.74 g, 20.86 mmol, 1.0 equiv) were added to a dry tube equipped with a magnetic stir bar. The tube was evacuated and backfilled with nitrogen, the evacuation and back fill procedure was repeated for another twice. Then N,N-Diisopropylethylamine (DIPEA) (4.49 g, 34.76 mmol, 2.0 equiv) and DMSO (25 mL) were added under a nitrogen atmosphere. The tube was sealed and the mixture was stirred at a temperature of 100° C. for 1 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate (10:1) as eluent to obtain the desired product (R)-2MeBn-OH as a brown-yellow oily liquid 2.6 g in 39% yield. ¹H NMR (500 MHz, CDCl₃): δ(ppm) 1.38 (s, 3H), 1.45 (s, 3H), 2.25 (s, 1H), 2.61 (dd, J=14.0, 10.5 Hz, 1H), 3.13 (dd, J=14.0, 3.5 Hz, 1H), 4.52 (s, 1H), 6.46 (dd, J=8.0, 7.5 Hz, 1H), 6.93-6.97 (m, 1H), 6.99-7.03 (m, 4H), 7.44 (dd, J=8.0, 1.5 Hz, 1H), 7.67 (d, J=8.5 Hz, 1H).
(2) Synthesis of (R)-2MeBn-NO₂: Pd(OAc)₂ (47 mg, 0.21 mmol, 3 mol %), SPhos (168 mg, 0.41 mmol, 6 mol %) and K₃PO₄ (2.90 g, 13.72 mmol, 2.0 equiv) were added to a dry tube equipped with a magnetic stir bar. The tube was evacuated and backfilled with nitrogen, the evacuation and back fill procedure was repeated for another twice. Then (R)-2MeBn-OH (2.6 g, 6.86 mmol, 1.0 equiv) and toluene (20 mL) were added under a nitrogen atmosphere. The tube was sealed and the mixture was stirred at a temperature of 100-110° C. for 1 days, cooled down to ambient temperature. The solvent was removed under reduced pressure and the residue was purified through column chromatography on silica gel using petroleum ether/ethyl acetate (20:1) as eluent to obtain the desired product (R)-2MeBn-NO₂ as a yellow oily liquid 1.80 g in 90% yield. ¹H NMR (500 MHz, CDCl₃): δ(ppm) 1.31 (s, 3H), 1.50 (s, 3H), 2.51 (dd, J=13.0, 11.5 Hz, 1H), 3.09 (dd, J=13.5, 3.7 Hz, 1H), 3.49-3.52 (m, 1H), 6.55 (dd, J=9.5, 8.0 Hz, 1H), 8.95-8.97 (m, 1H), 7.26-7.28 (m, 2H), 7.30-7.33 (m, 1H), 7.39-7.42 (m, 2H), 7.70 (dd, J=8.5, 1.5 Hz, 1H), 7.74 (s, 1H).
(3) Synthesis of (R)-2MeBn-NH₂: (R)-2MeBn-NO₂ (1.70 g, 5.70 mmol, 1.0 equiv), SnCl₂·2H₂O (5.14 g, 22.80 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (10 mL:10 mL) were added under a nitrogen atmosphere. The flask was sealed and the mixture was stirred at a temperature of 78° C. for 2 days, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/ethyl acetate (10:1-6:1) as eluent to obtain the desired product (R)-2MeBn-NH₂ as a white oily liquid 1.40 g in 97% yield. ¹H NMR (500 MHz, CDCl₃): δ(ppm) 1.34 (s, 3H), 1.46 (s, 3H), 2.49 (dd, J=13.5, 11.5 Hz, 1H), 3.04 (dd, J=13.5, 3.0 Hz, 1H), 3.17 (s, 3H), 3.26 (dd, J=11.0, 3.0 Hz, 1H), 6.29 (dd, J=8.0, 1.5 Hz, 1H), 6.40 (dd, J=8.0, 1.5 Hz, 1H), 6.60 (t, J=8.0 Hz, 1H), 7.23-7.25 (m, 2H)), 7.26-7.29 (m, 1H)), 7.34-7.37 (m, 2H).
(4) Synthesis of (R)-2MeBn-L9: (R)-2MeBn-NH₂ (763 mg, 3.0 mmol, 1.0 equiv), 1-Cl (1.45 g, 3.0 mmol, 1.0 equiv), Pd₂(dba)₃ (43 mg, 0.09 mmol, 3 mol %), JohnPhos (54 mg, 0.18 mmol, 6 mol %) and tBuONa (577 mg, 6.0 mmol, 2.0 equiv) were added to a dry tube equipped with a magnetic stir bar. The tube was evacuated and backfilled with nitrogen, the evacuation and back fill procedure was repeated for another twice. Then toluene (15 mL) was added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (20:1) as eluent to obtain the desired product (R)-2MeBn-L9 as a yellow foamy solid 1.8 g in 84% yield. ¹H NMR (500 MHz, DMSO-d₆): δ(ppm) 1.21 (m, 9H), 1.22 (s, 3H), 1.26 (s, 3H), 1.27 (s, 9H), 2.19 (dd, J=12.5, 10.5 Hz, 1H), 2.85 (dd, J=13.5, 3.0 Hz, 1H), 3.02 (d, J=9.0 Hz, 1H), 3.88 (d, J=1.0 Hz, 1H), 5.97 (t, J=2.0 Hz, 1H), 6.39 (t, J=2.0 Hz, 1H), 6.42-6.45 (m, 2H), 6.50 (dd, J=6.0, 3.5 Hz, 1H), 6.52 (t, J=1.5 Hz, 1H), 6.92 (dd, J=6.0, 2.0 Hz, 2H), 7.03 (dd, J=8.5, 2.5 Hz, 1H), 7.08-7.10 (m, 3H), 7.30-7.38 (m, 1H), 7.35 (d, J=2.0 Hz, 1H), 7.38 (s, 1H), 7.41-7.44 (m, 2H), 7.59 (d, J=1.5 Hz, 1H), 7.73 (d, J=8.5 Hz, 1H), 8.20 (t, J=8.0 Hz, 2H), 8.54 (d, J=5.5 Hz, 1H).
(5) Synthesis of (R)-2MeBn-LA9: (R)-2MeBn-L9 (1.8 g, 1.0 mmol, 1.0 equiv), NH₄PF₆ (822 mg, 5.04 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (6 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (5:1) first, then dichloromethane/ethyl acetate (40:1) as eluent to obtain the desired product (R)-2MeBn-LA9 as a yellow foamy solid 711 mg in 32% yield. ¹H NMR (500 MHz, DMSO-d₆): δ(ppm) 1.31 (s, 9H), 1.33 (s, 12H), 1.73 (s, 3H), 2.64 (dd, J=13.5, 10.5 Hz, 1H), 3.47 (dd, J=14, 5.0 Hz, 1H), 5.08 (dd, J=10.5, 5.0 Hz, 1H), 6.85 (d, J=7.0 Hz, 2H), 6.88 (t, J=2.0 Hz, 1H), 7.00 (t, J=7.5 Hz, 1H), 7.08-7.14 (m, 3H), 7.16-7.21 (m, 2H), 7.25 (d, J=8.5 Hz, 1H), 7.33-7.36 (m, 1H), 7.45-7.50 (m, 3H), 7.52 (d, J=2.0 Hz, 1H), 7.56 (t, J=8.5 Hz, 1H), 7.68 (d, J=1.5 Hz, 1H), 7.74 (d, J=8.5 Hz, 1H), 8.26 (d, J=7.5 Hz, 1H), 8.35 (d, J=8.5 Hz, 1H), 8.59 (d, J=5.5 Hz, 1H), 9.10 (s, 1H).
(6) Synthesis of (R)-2MeBn-M-PtA9: (R)-2MeBn-LA9 (300 mg, 0.34 mmol, 1.0 equiv), Pt(COD)Cl₂ (135 mg, 0.36 mmol, 1.05 equiv) and NaOAc (84 mg, 1.02 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (20 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (R)-2MeBn-M-PtA9 as a pale yellow solid 247 mg in 79% yield. ¹H NMR (500 MHz, DMSO-d₆): δ(ppm) 1.41 (s, 9H), 1.44 (s, 9H), 1.54 (s, 3H), 1.59 (s, 3H), 2.60-2.65 (m, 1H), 3.20 (dd, J=14.5, 6.5 Hz, 1H), 4.94 (t, J=6.5 Hz, 1H), 6.53 (t, J=7.5 Hz, 1H), 6.56 (d, J=7.5 Hz, 2H), 6.71 (t, J=8.0 Hz, 2H), 6.93 (d, J=7.5 Hz, 1H), 6.96 (d, J=1.5 Hz, 1H), 7.24 (d, J=7.5 Hz, 1H), 7.42 (t, J=8.0 Hz, 2H), 7.51 (dd, J=5.5, 1.5 Hz, 2H), 7.54-7.57 (m, 1H), 7.75 (d, J=8.0 Hz, 1H), 7.88 (d, J=8.0 Hz, 1H), 8.15 (d, J=8.0 Hz, 1H), 8.18 (d, J=7.0 Hz, 1H), 8.22 (d, J=2.0 Hz, 1H), 9.42 (d, J=6.5 Hz, 1H).

### Example 19

The synthetic route for (S)-2MeBn-P—PtA9 is as follows:

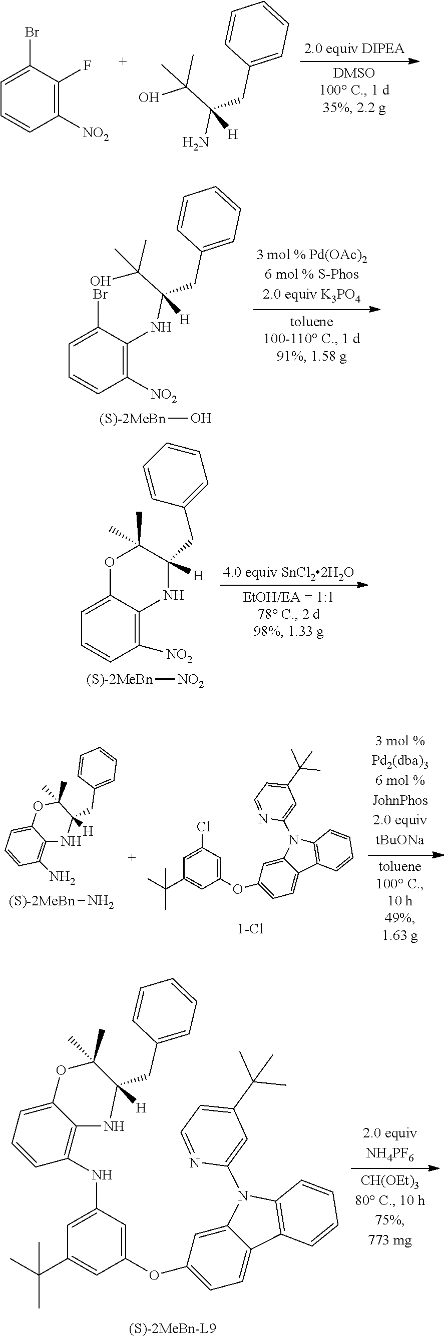

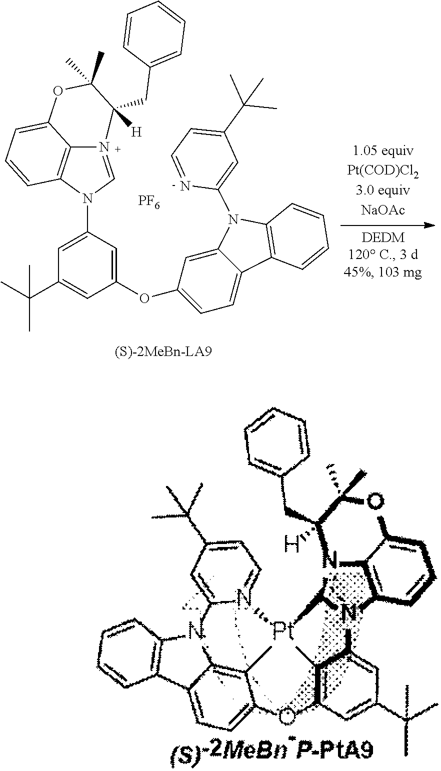

(1) Synthesis of (S)-2MeBn-OH: 1-bromo-2-fluoro-3-nitrobenzene (3.69 g, 16.77 mmol, 1.0 equiv), (S)-3-amino-2-methyl-4-phenylbutan-2-ol (3.61 g, 20.12 mmol, 1.0 equiv) were added to a dry tube equipped with a magnetic stir bar. The tube was evacuated and backfilled with nitrogen, the evacuation and back fill procedure was repeated for another twice. Then N,N-Diisopropylethylamine (DIPEA) (4.33 g, 33.54 mmol, 2.0 equiv) and DMSO (40 mL) were added under a nitrogen atmosphere. The tube was sealed and the mixture was stirred at a temperature of 100° C. for 1 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate (10:1) as eluent to obtain the desired product (S)-2MeBn-OH as a brown-yellow oily liquid 2.2 g in 35% yield. ¹H NMR (400 MHz, DMSO-d₆): δ(ppm) 1.22 (s, 3H), 1.27 (s, 3H), 2.57 (dd, J=13.6, 9.6 Hz, 1H), 3.10 (dd, J=13.6, 4.0 Hz, 1H), 4.19 (s, 1H), 4.90 (s, 1H), 6.54 (t, J=8.0 Hz, 1H), 6.94-6.98 (m, 1H), 7.01-7.04 (m, 4H), 7.56 (dd, J=8.0, 1.6 Hz, 1H), 7.63 (d, J=8.0 Hz, 1H).
(2) Synthesis of (S)-2MeBn-NO₂: Pd(OAc)₂ (38 mg, 0.17 mmol, 3 mol %), SPhos (144 mg, 0.35 mmol, 6 mol %) and K₃PO₄ (2.46 g, 11.60 mmol, 2.0 equiv) were added to a dry tube equipped with a magnetic stir bar. The tube was evacuated and backfilled with nitrogen, the evacuation and back fill procedure was repeated for another twice. Then (S)-2MeBn-OH (2.2 g, 5.80 mmol, 1.0 equiv) and toluene (30 mL) were added under a nitrogen atmosphere. The tube was sealed and the mixture was stirred at a temperature of 100-110° C. for 1 days, cooled down to ambient temperature. The solvent was removed under reduced pressure and the residue was purified through column chromatography on silica gel using petroleum ether/ethyl acetate (20:1) as eluent to obtain the desired product (S)-2MeBn-NO₂ as a yellow oily liquid 1.58 g in 91% yield. ¹H NNR (500 MHz, CDCl₃): δ(ppm) 1.31 (s, 3H), 1.50 (s, 3H), 2.51 (dd, J=13.5, 11.5 Hz, 1H), 3.09 (dd, J=13.0, 3.0 Hz, 1H), 3.49-3.52 (m, 1H), 6.55 (dd, J=9.0, 8.0 Hz, 1H), 6.96 (d, J=8.0 Hz, 1H), 7.26 (s, 1H), 7.28 (s, 1H), 7.31 (t, J=7.5 Hz, 1H), 7.40 (t, J=7.5 Hz, 2H), 7.70 (dd, J=9.0, 1.5 Hz, 1H), 7.74 (s, 1H).
(3) Synthesis of (S)-2MeBn-NH₂: (S)-2MeBn-NO₂ (1.66 g, 5.36 mmol, 1.0 equiv), SnCl₂·2H₂O (4.41 g, 21.44 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (20 mL:20 mL) were added under a nitrogen atmosphere. The flask was sealed and the mixture was stirred at a temperature of 78° C. for 2 days, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/ethyl acetate (10:1-6:1) as eluent to obtain the desired product (S)-2MeBn-NH₂ as a white oily liquid 1.33 g in 98% yield. ¹H NMR (500 MHz, CDCl₃): δ(ppm) 1.33 (s, 3H), 1.45 (s, 3H), 2.48 (dd, J=13.5, 11.0 Hz, 1H), 3.03 (dd, J=13.5, 3.0 Hz, 1H), 3.13-3.18 (m, 3H), 3.25 (dd, J=11.0, 3.0 Hz, 1H), 6.28 (dd, J=7.5, 1.0 Hz, 1H), 6.38 (dd, J=8.0, 1.0 Hz, 1H), 6.59 (t, J=8.0 Hz, 1H), 7.23 (d, J=7.0 Hz, 2H), 7.25-7.28 (m, 1H), 7.33-7.36 (m, 2H).
(4) Synthesis of (S)-2MeBn-L9: (S)-2MeBn-NH₂ (1.18 g, 4.64 mmol, 1.0 equiv), 1-Cl (2.24 g, 4.64 mmol, 1.0 equiv), Pd₂(dba)₃ (128 mg, 0.14 mmol, 3 mol %), JohnPhos (84 mg, 0.28 mmol, 6 mol %) and tBuONa (892 mg, 9.28 mmol, 2.0 equiv) were added to a dry tube equipped with a magnetic stir bar. The tube was evacuated and backfilled with nitrogen, the evacuation and back fill procedure was repeated for another twice. Then toluene (20 mL) was added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (20:1) as eluent to obtain the desired product (S)-2MeBn-L9 as a yellow foamy solid 1.63 g in 49% yield. ¹H NMR (500 MHz, DMSO-d₆): δ(ppm) 1.07 (s, 3H), 1.22 (s, 9H), 1.28 (s, 12H), 2.20 (dd, J=13.0, 10.5 Hz, 1H), 2.86 (dd, J=13.0, 3.0 Hz, 1H), 3.02-3.05 (m, 1H), 3.90 (d, J=1.5 Hz, 1H), 5.99 (t, J=2.0 Hz, 1H), 6.40 (t, J=2.0 Hz, 1H), 6.42-6.46 (m, 2H), 6.51 (dd, J=6.0, 3.5 Hz, 1H), 6.53 (t, J=1.5 Hz, 1H), 6.93 (dd, J=5.5, 2.0 Hz, 2H), 7.04 (dd, J=8.5, 2.5 Hz, 1H), 7.08-7.11 (m, 3H), 7.31-7.34 (m, 1H), 7.36 (d, J=2.5 Hz, 1H), 7.38 (s, 1H), 7.42-7.45 (m, 2H), 7.60 (d, J=1.0 Hz, 1H), 7.73 (d, J=8.0 Hz, 1H), 8.21 (t, J=7.5 Hz, 2H), 8.55 (d, J=5.5 Hz, 1H).
(5) Synthesis of (S)-2MeBn-LA9: (S)-2MeBn-L9 (715 mg, 1.0 mmol, 1.0 equiv), NH₄PF₆ (326 mg, 2.0 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (4 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (5:1) first, then dichloromethane/ethyl acetate (40:1) as eluent to obtain the desired product (S)-2MeBn-LA9 as a yellow foamy solid 773 mg in 75% yield. ¹H NMR (500 MHz, DMSO-d₆): δ(ppm) 1.31 (s, 9H), 1.33 (s, 3H), 1.33 (s, 9H), 1.73 (s, 3H), 2.64 (dd, J=13.5, 10.5 Hz, 1H), 3.47 (dd, J=14.0, 5.0 Hz, 1H), 5.08 (dd, J=10.5, 5.0 Hz, 1H), 6.85 (d, J=7.0 Hz, 2H), 6.89 (d, J=2.0 Hz, 1H), 7.01 (t, J=7.5 Hz, 1H), 7.10-7.13 (m, 3H), 7.17-7.20 (m, 2H), 7.25 (d, J=8.5 Hz, 1H), 7.33-7.36 (m, 1H), 7.45-7.50 (m, 3H), 7.52 (d, J=2.0 Hz, 1H), 7.56 (t, J=8.0 Hz, 1H), 7.68 (d, J=1.0 Hz, 1H), 7.74 (d, J=8.5 Hz, 1H), 8.26 (d, J=7.5 Hz, 1H), 8.35 (d, J=8.5 Hz, 1H), 8.59 (d, J=5.5 Hz, 1H), 9.10 (s, 1H).
(6) Synthesis of (S)-2MeBn-P—PtA9: (S)-2MeBn-LA9 (200 mg, 0.23 mmol, 1.0 equiv), Pt(COD)Cl₂ (90 mg, 0.24 mmol, 1.05 equiv) and NaOAc (57 mg, 0.69 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (14 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (S)-2MeBn-P—PtA9 as a pale yellow solid 103 mg in 45% yield. ¹H NMR (500 MHz, DMSO-d₆): δ(ppm) 1.39 (s, 9H), 1.44 (s, 9H), 1.53 (s, 3H), 1.58 (s, 3H), 2.62 (dd, J=14.5, 6.0 Hz, 1H), 3.20 (dd, J=15.0, 7.0 Hz, 1H), 4.94 (t, J=6.5 Hz, 1H), 6.51-6.56 (m, 3H), 6.71 (t, J=7.5 Hz, 2H), 6.93 (d, J=8.0 Hz, 1H), 6.96 (d, J=1.5 Hz, 1H), 7.24 (d, J=8.5 Hz, 1H), 7.42 (t, J=8.0 Hz, 2H), 7.49-7.50 (m, 2H), 7.54-7.57 (m, 1H), 7.75 (d, J=8.5 Hz, 1H), 7.88 (d, J=8.0 Hz, 1H), 8.14 (d, J=8.0 Hz, 1H), 8.18 (d, J=7.5 Hz, 1H), 8.21 (d, J=2.0 Hz, 1H), 9.41 (d, J=6.5 Hz, 1H).

### Example 20

The synthetic route for (R)-Ph-M-PtA10 is as follows:

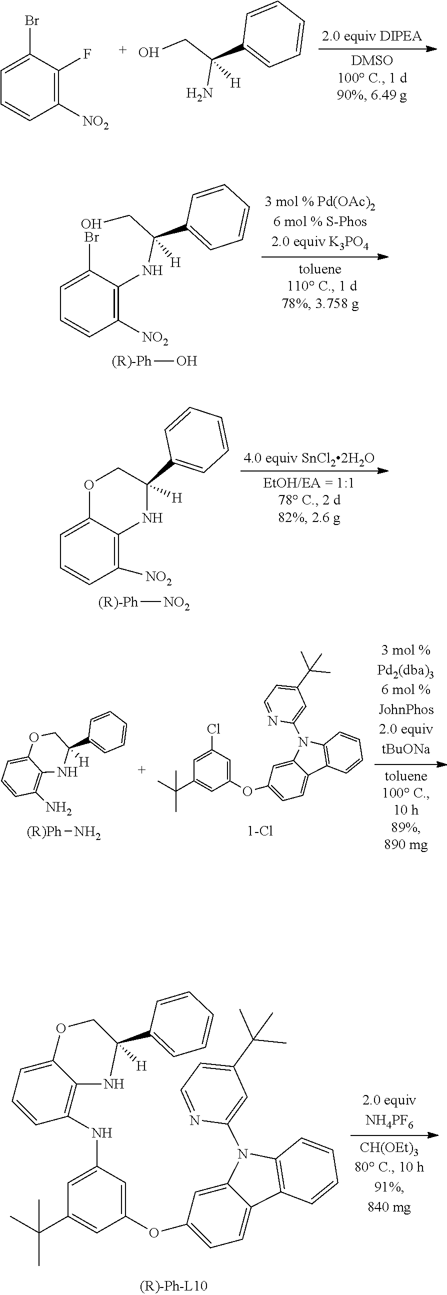

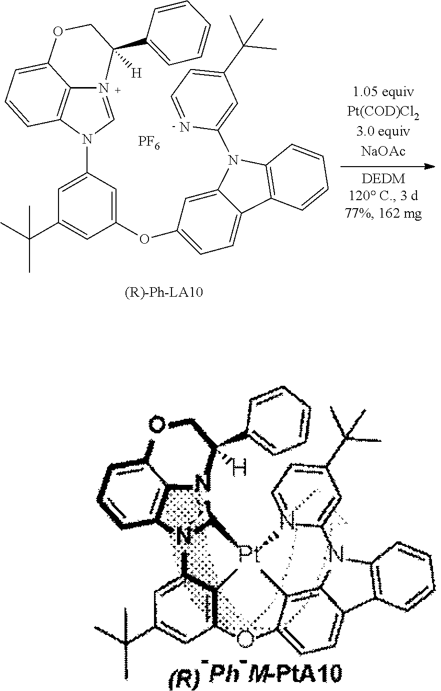

(1) Synthesis of (R)-Ph-OH: 1-bromo-2-fluoro-3-nitrobenzene (4.69 g, 21.3 mmol, 1.0 equiv), (R)-2-amino-2-phenylethan-1-ol (2.34 g, 22.73 mmol, 1.0 equiv) were added to a dry tube equipped with a magnetic stir bar. The tube was evacuated and backfilled with nitrogen, the evacuation and back fill procedure was repeated for another twice. Then N,N-Diisopropylethylamine (DIPEA) (5.5 g, 42.6 mmol, 2.0 equiv) and DMSO (25 mL) were added under a nitrogen atmosphere. The tube was sealed and the mixture was stirred at a temperature of 100° C. for 1 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate (20:1-5:1) as eluent to obtain the desired product (R)-Ph-OH as a red oily liquid 6.49 g in 90% yield. ¹H NMR (500 MHz, DMSO-d₆): δ(ppm) 3.58 (m, 1H), 3.80 (dt, J=11.0, 4.0 Hz, 1H), 4.74-4.78 (m, 1H), 5.30 (t, J=5.0 Hz, 1H), 6.75-6.81 (m, 2H), 7.13-7.15 (m, 2H), 7.16-7.20 (m, 1H), 7.22-7.24 (m, 1H), 7.24-7.26 (m, 1H), 7.75 (dd, J=8.0, 1.5 Hz, 1H), 7.83 (dd, J=8.0, 1.5 Hz, 1H).
(2) Synthesis of (R)-Ph-NO₂: Pd(OAc)₂ (0.125 g, 0.558 mmol, 3 mol %), SPhos (0.458 g, 1.116 mmol, 6 mol %) and K₃PO₄ (7.89 g, 37.2 mmol, 2.0 equiv) were added to a dry tube equipped with a magnetic stir bar. The tube was evacuated and backfilled with nitrogen, the evacuation and back fill procedure was repeated for another twice. Then (R)-Ph-OH (6.3 g, 18.6 mmol, 1.0 equiv) and toluene (50 mL) were added under a nitrogen atmosphere. The tube was sealed and the mixture was stirred at a temperature of 110° C. for 1 days, cooled down to ambient temperature. The solvent was removed under reduced pressure and the residue was purified through column chromatography on silica gel using petroleum ether/ethyl acetate (50:1) as eluent to obtain the desired product (R)-Ph-NO₂ as a red oily liquid 3.75 g in 78% yield. ¹H NMR (500 MHz, DMSO-d₆): δ(ppm) 4.21 (dd, J=10.5, 4.5 Hz, 1H), 4.31 (dd, J=10.5, 3.0 Hz, 1H), 4.91 (dd, J=7.0, 3.5 Hz, 1H), 6.62 (dd, J=8.5, 7.5 Hz, 1H), 7.08 (d, J=7.6 Hz, 1H), 7.29-7.34 (m, 1H), 7.34-7.37 (m, 3H), 7.37-7.40 (m, 1H), 7.72 (dd, J=9.0, 1.5 Hz, 1H), 8.49 (d, J=2.5 Hz, 1H).
(3) Synthesis of (R)-Ph-NH₂: (R)-Ph-NO₂ (3.6 g, 25.5 mmol, 1.0 equiv), SnCl₂·2H₂O (12.6 g, 0.102 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (40 mL:40 mL) were added under a nitrogen atmosphere. The flask was sealed and the mixture was stirred at a temperature of 78° C. for 2 days, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/ethyl acetate (10:1-5:1) as eluent to obtain the desired product (R)-Ph-NH₂ as a light yellow oily liquid 2.6 g in 82% yield. ¹H NMR (500 MHz, DMSO-d₆): δ(ppm) 3.87 (dd, J=10.5, 7.5 Hz, 1H), 4.17 (ddd, J=10.5, 3.0, 1.5 Hz, 1H), 4.45 (dt, J=7.5, 2.5 Hz, 1H), 4.65 (s, 2H), 5.03 (s, 1H), 6.07 (dd, J=8.0, 1.0 Hz, 1H), 6.21 (dd, J=7.5, 1.5 Hz, 1H), 6.36 (t, J=7.5 Hz, 1H), 7.29-7.34 (m, 1H), 7.35-7.41 (m, 2H), 7.43-7.47 (m, 2H).
(4) Synthesis of (R)-Ph-L10: (R)-Ph-NH₂ (339 mg, 1.5 mmol, 1.0 equiv), 1-Cl (724 mg, 1.5 mmol, 1.0 equiv), Pd₂(dba)₃ (41 mg, 0.045 mmol, 3 mol %), JohnPhos (27 mg, 0.09 mmol, 6 mol %) and tBuONa (288 mg, 3.0 mmol, 2.0 equiv) were added to a dry tube equipped with a magnetic stir bar. The tube was evacuated and backfilled with nitrogen, the evacuation and back fill procedure was repeated for another twice. Then toluene (5 mL) was added under a nitrogen atmosphere. The tube was sealed and the mixture was stirred at a temperature of 110° C. for 10 hours, cooled down to ambient temperature. The solvent was removed under reduced pressure and the residue was purified through column chromatography on silica gel using petroleum ether/ethyl acetate (30:1) as eluent to obtain the desired product (R)-Ph-L10 as a yellow foamy solid 890 mg in 89% yield.
(5) Synthesis of (R)-Ph-LA10: (R)-Ph-L10 (750 mg, 1.11 mmol, 1.0 equiv), NH₄PF₆ (360 mg, 2.22 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (8 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (10:1) first, then dichloromethane/ethyl acetate (10:1) as eluent to obtain the desired product (R)-Ph-LA10 as a gray-white, foamy solid 840 mg in 91% yield. ¹H NMR (500 MHz, DMSO-d₆): δ(ppm) 1.29 (s, 9H), 1.33 (s, 9H), 4.03 (q, J=7.0 Hz, 1H), 4.56 (dd, J=12.0, 8.0 Hz, 1H), 4.82 (dd, J=12.0, 3.5 Hz, 1H), 6.03 (dd, J=7.5, 3.5 Hz, 1H), 7.19 (dd, J=8.5, 2.0 Hz, 1H), 7.21 (t, J=2.0 Hz, 1H), 7.27 (d, J=8.0 Hz, 1H), 7.32-7.37 (m, 1H), 7.41 (d, J=8.5 Hz, 1H), 7.44-7.51 (m, 7H), 7.53 (t, J=1.5 Hz, 1H), 7.54-7.62 (m, 3H), 7.66 (d, J=1.0 Hz, 1H), 7.74 (d, J=8.5 Hz, 1H), 8.24 (d, J=7.5 Hz, 1H), 8.31 (d, J=8.5 Hz, 1H), 8.52-8.62 (m, 1H), 10.08 (s, 1H).
(6) Synthesis of (R)-Ph-M-PtA10: (R)-Ph-LA10 (200 mg, 0.24 mmol, 1.0 equiv), Pt(COD)Cl₂ (94 mg, 0.252 mmol, 1.05 equiv) and NaOAc (60 mg, 0.72 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (15 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (10:1-1:1) as eluent to obtain the desired product (R)-Ph-M-PtA10 as a pale yellow solid 162 mg in 77% yield. ¹H NMR (500 MHz, DMSO-d₆): δ(ppm) 1.34 (s, 9H), 1.46 (s, 9H), 4.35 (m, 1H), 4.71 (s, 1H), 6.10 (dd, J=5.5, 3.5 Hz, 1H), 6.74-7.34 (m, 9H), 7.37 (t, J=7.5 Hz, 1H), 7.45 (t, J=8.0 Hz, 1H), 7.51 (td, J=6.0, 1.5 Hz, 1H), 7.58 (s, 1H), 7.83 (t, J=8.5 Hz, 2H), 7.86-8.00 (m, 2H), 8.10 (d, J=7.7 Hz, 1H), 9.2-9.5 (m, 1H).

### Example 21

The synthetic route for (S)-Ph-P—PtA10 is as follows:

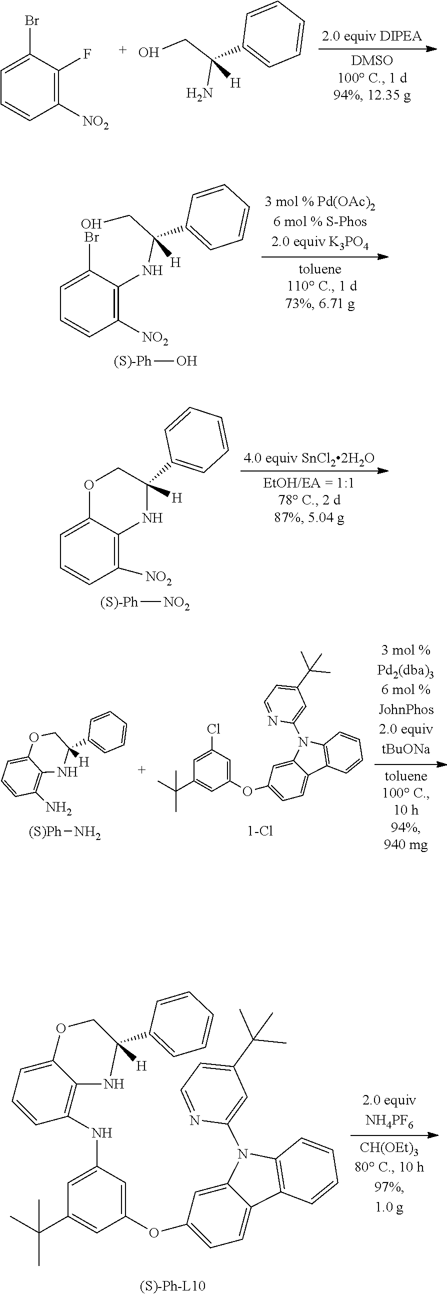

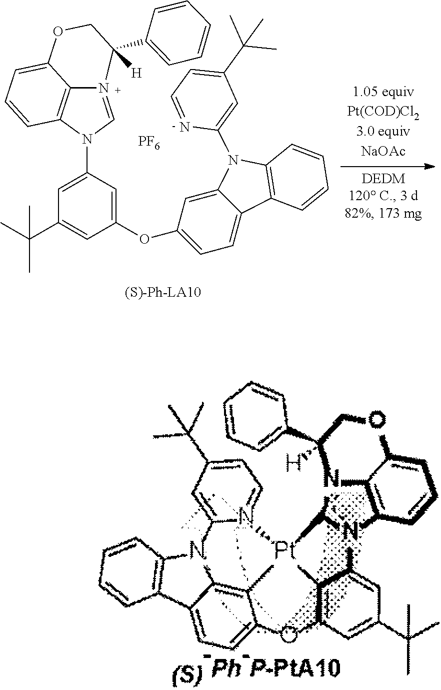

(1) Synthesis of (S)-Ph-OH: 1-bromo-2-fluoro-3-nitrobenzene (8.52 g, 38 mmol, 1.0 equiv), (S)-2-amino-2-phenylethan-1-ol (5.2 g, 38 mmol, 1.0 equiv) were added to a dry tube equipped with a magnetic stir bar. The tube was evacuated and backfilled with nitrogen, the evacuation and back fill procedure was repeated for another twice. Then N,N-Diisopropylethylamine (DIPEA) (9.3 g, 72 mmol, 2.0 equiv) and DMSO (60 mL) were added under a nitrogen atmosphere. The tube was sealed and the mixture was stirred at a temperature of 100° C. for 1 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate (5:1-2:1) as eluent to obtain the desired product (S)-Ph-OH as a red oily liquid 12.35 g in 94% yield. ¹H NMR (400 MHz, DMSO-d₆): δ(ppm) 3.54-3.60 (m, 1H), 3.80 (dt, J=10.8, 4.8 Hz, 1H), 4.73-4.77 (m, 1H), 5.31 (t, J=5.2 Hz, 1H), 6.74-6.82 (m, 2H), 7.15 (tt, J=5.8, 1.4 Hz, 2H), 7.17-7.21 (m, 1H), 7.21-7.27 (m, 2H), 7.74 (dd, J=8.4, 1.6 Hz, 1H), 7.83 (dd, J=7.6, 1.6 Hz, 1H).
(2) Synthesis of (S)-Ph-NO₂: Pd(OAc)₂ (0.24 g, 1.07 mmol, 3 mol %), SPhos (0.88 g, 2.14 mmol, 6 mol %) and K₃PO₄ (15.2 g, 71.6 mmol, 2.0 equiv) were added to a dry tube equipped with a magnetic stir bar. The tube was evacuated and backfilled with nitrogen, the evacuation and back fill procedure was repeated for another twice. Then (S)-Ph-OH (12.1 g, 35.8 mmol, 1.0 equiv) and toluene (100 mL) were added under a nitrogen atmosphere. The tube was sealed and the mixture was stirred at a temperature of 110° C. for 1 days, cooled down to ambient temperature. The solvent was removed under reduced pressure and the residue was purified through column chromatography on silica gel using petroleum ether/ethyl acetate (30:1-20:1) as eluent to obtain the desired product (S)-Ph-NO₂ as a red oily liquid 6.71 g in 73% yield. ¹H NMR (500 MHz, DMSO-d₆): δ(ppm) 4.20 (dd, J=11.0, 4.5 Hz, 1H), 4.31 (dd, J=10.5, 3.0 Hz, 1H), 4.91 (dd, J=7.5, 3.0 Hz, 1H), 6.60-6.63 (m, 1H), 7.08 (ddd, J=7.5, 1.5, 0.5 Hz, 1H), 7.29-7.33 (m, 1H), 7.34-7.37 (m, 3H), 7.37-7.41 (m, 1H), 7.72 (dd, J=9.0, 1.5 Hz, 1H), 8.49 (d, J=3.0 Hz, 1H).
(3) Synthesis of (S)-Ph-NH₂: (S)-Ph-NO₂ (6.54 g, 25.5 mmol, 1.0 equiv), SnCl₂·2H₂O (23.02 g, 0.102 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (1:1) were added under a nitrogen atmosphere. The flask was sealed and the mixture was stirred at a temperature of 78° C. for 2 days, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/ethyl acetate (10:1-5:1) as eluent to obtain the desired product (S)-Ph-NH₂ as a light yellow oily liquid 5.04 g in 87% yield. ¹H NMR (500 MHz, DMSO-d₆): δ(ppm) 3.88 (dd, J=10.5, 8.0 Hz, 1H), 4.18 (dd, J=10.5, 2.5 Hz, 1H), 4.46 (d, J=7.5 Hz, 1H), 4.66 (s, 2H), 5.04 (s, 1H), 6.09 (dd, J=8.0, 1.5 Hz, 1H), 6.22 (dd, J=8.0, 1.5 Hz, 1H), 6.37 (t, J=8.0 Hz, 1H), 7.30-7.34 (m, 1H), 7.38 (t, J=7.5 Hz, 2H), 7.44-7.48 (m, 2H).
(4) Synthesis of (S)-Ph-L10: (S)-Ph-NH₂ (339 mg, 1.5 mmol, 1.0 equiv), 1-Cl (724 mg, 1.5 mmol, 1.0 equiv), Pd₂(dba)₃ (41 mg, 0.045 mmol, 3 mol %), JohnPhos (27 mg, 0.09 mmol, 6 mol %) and tBuONa (288 mg, 3.0 mmol, 2.0 equiv) were added to a dry tube equipped with a magnetic stir bar. The tube was evacuated and backfilled with nitrogen, the evacuation and back fill procedure was repeated for another twice. Then toluene (5 mL) was added under a nitrogen atmosphere. The tube was sealed and the mixture was stirred at a temperature of 110° C. for 10 hours, cooled down to ambient temperature. The solvent was removed under reduced pressure and the residue was purified through column chromatography on silica gel using petroleum ether/ethyl acetate (30:1) as eluent to obtain the desired product (S)-Ph-L10 as a yellow foamy solid 940 mg in 94% yield.
(5) Synthesis of (S)-Ph-LA10: (S)-Ph-L10 (840 mg, 1.24 mmol, 1.0 equiv), NH₄PF₆ (430 mg, 2.66 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (8 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (30:1) first, then dichloromethane/ethyl acetate (10:1) as eluent to obtain the desired product (S)-Ph-LA10 as a yellow foamy solid 1.0 g in 97% yield. ¹H NMR (500 MHz, DMSO-d₆): δ(ppm) 1.29 (s, 9H), 1.33 (s, 9H), 4.56 (dd, J=12.0, 8.0 Hz, 1H), 4.82 (dd, J=12.0, 3.5 Hz, 1H), 6.03 (dd, J=7.5, 3.5 Hz, 1H), 7.19 (dd, J=8.5, 2.0 Hz, 1H), 7.21 (t, J=2.0 Hz, 1H), 7.27 (d, J=8.0 Hz, 1H), 7.34 (td, J=8.5, 1.0 Hz, 1H), 7.41 (d, J=8.5 Hz, 1H), 7.44-7.51 (m, 7H), 7.53 (t, J=1.5 Hz, 1H), 7.54-7.61 (m, 3H), 7.66 (d, J=1.0 Hz, 1H), 7.74 (d, J=8.5 Hz, 1H), 8.24 (d, J=7.5 Hz, 1H), 8.31 (d, J=8.5 Hz, 1H), 8.55-8.62 (m, 1H), 10.08 (s, 1H).
(6) Synthesis of (S)-Ph-P—PtA10: (S)-Ph-LA10 (200 mg, 0.24 mmol, 1.0 equiv), Pt(COD)Cl₂ (94 mg, 0.252 mmol, 1.05 equiv) and NaOAc (60 mg, 0.72 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (15 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (10:1-1:1) as eluent to obtain the desired product (S)-Ph-P—PtA10 as a pale yellow solid 173 mg in 82% yield. ¹H NMR (500 MHz, DMSO-d₆): δ (ppm) 1.35 (s, 9H), 1.46 (s, 9H), 4.35 (m, 1H), 4.72 (s, 1H), 6.12 (dd, J=5.5, 4.0 Hz, 1H), 6.84-7.32 (m, 9H), 7.38 (t, J=7.5 Hz, 1H), 7.46 (t, J=8.5 Hz, 1H), 7.48-7.54 (m, 1H), 7.59 (s, 1H), 7.84 (dd, J=12.5, 8.5 Hz, 2H), 7.87-7.99 (m, 2H), 8.11 (d, J=7.5 Hz, 1H), 9.2-9.5 (m, 1H).

### Example 22

The synthetic route for (S,R)-2Ph-M-PtA11 is as follows:

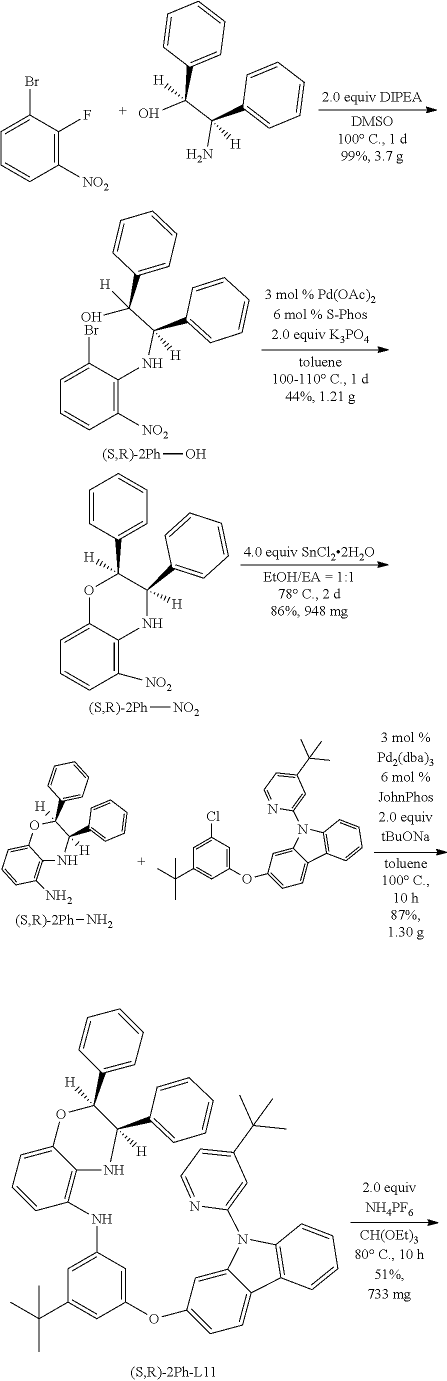

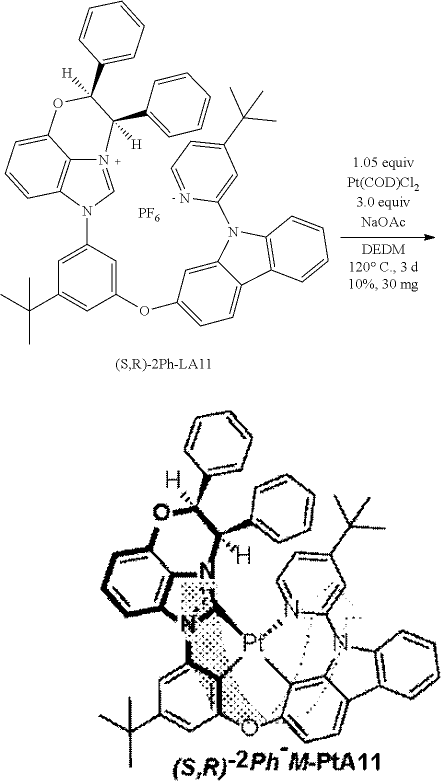

(1) Synthesis of (S,R)-2Ph-OH: 1-bromo-2-fluoro-3-nitrobenzene (2.0 g, 9.09 mmol, 1.0 equiv), (1S,2R)-2-amino-1,2-diphenylethan-1-ol (1.94 g, 9.09 mmol, 1.0 equiv) were added to a dry tube equipped with a magnetic stir bar. The tube was evacuated and backfilled with nitrogen, the evacuation and back fill procedure was repeated for another twice. Then N,N-Diisopropylethylamine (DIPEA) (2.35 g, 18.18 mmol, 2.0 equiv) and DMSO (15 mL) were added under a nitrogen atmosphere. The tube was sealed and the mixture was stirred at a temperature of 100° C. for 1 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate (15:1-5:1) as eluent to obtain the desired product (S,R)-2Ph-OH as a brown-yellow oily liquid 3.7 g in 99% yield. ¹H NMR (500 MHz, DMSO-d₆): δ(ppm) 4.92 (dd, J=9.5, 4.0 Hz, 1H), 5.14 (t, J=4.0 Hz, 1H), 6.13 (d, J=4.0 Hz, 1H), 6.78 (t, J=8.0 Hz, 1H), 6.82 (dd, J=8.0, 2.0 Hz, 2H), 7.02-7.07 (m, 5H), 7.11-7.19 (m, 4H), 7.72 (dd, J=8.5, 1.5 Hz, 1H), 7.82 (dd, J=7.5, 1.5 Hz, 1H).
(2) Synthesis of (S,R)-2Ph-NO₂: Pd(OAc)₂ (56 mg, 0.25 mmol, 3 mol %), SPhos (201 mg, 0.49 mmol, 6 mol %) and K₃PO₄ (3.6 g, 16.98 mmol, 2.0 equiv) were added to a dry tube equipped with a magnetic stir bar. The tube was evacuated and backfilled with nitrogen, the evacuation and back fill procedure was repeated for another twice. Then (S,R)-2Ph-OH (3.39 g, 8.22 mmol, 1.0 equiv) and toluene (40 mL) were added under a nitrogen atmosphere. The tube was sealed and the mixture was stirred at a temperature of 100-110° C. for 1 days, cooled down to ambient temperature. The solvent was removed under reduced pressure and the residue was purified through column chromatography on silica gel using petroleum ether/ethyl acetate/dichloromethane (10:1:1-6:1:1) as eluent to obtain the desired product (S,R)-2Ph-NO₂ as a yellow flake solid 1.21 g in 44% yield. ¹H NMR (500 MHz, DMSO-d₆): δ(ppm) 5.08 (dd, J=5.0, 3.0 Hz, 1H), 5.49 (d, J=2.5 Hz, 1H), 6.69 (dd, J=8.5, 7.5 Hz, 1H), 6.71-6.73 (m, 2H), 7.06-7.11 (m, 4H), 7.13-7.16 (m, 1H), 7.21-7.23 (m, 3H), 7.25-7.27 (m, 1H), 7.80 (dd, J=8.5, 1.5 Hz, 1H), 9.01 (d, J=5.0 Hz, 1H).
(3) Synthesis of (S,R)-2Ph-NH₂: (S,R)-2Ph-NO₂ (1.21 g, 3.64 mmol, 1.0 equiv), SnCl₂·2H₂O (3.29 g, 14.56 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (20 mL:20 mL) were added under a nitrogen atmosphere. The flask was sealed and the mixture was stirred at a temperature of 78° C. for 2 days, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/dichloromethane (2:1) as eluent to obtain the desired product (S,R)-2Ph-NH₂ as a yellow solid 5.04 g in 86% yield. ¹H NMR (500 MHz, DMSO-d₆): δ(ppm) 4.67 (s, 2H), 4.76 (t, J=3.0 Hz, 1H), 5.33 (d, J=3.0 Hz, 1H), 5.44 (d, J=3.0 Hz, 1H), 6.20 (dd, J=8.0, 1.5 Hz, 1H), 6.28 (dd, J=8.0, 1.5 Hz, 1H), 6.41 (t, J=7.5 Hz, 1H), 6.87 (dd, J=8.0, 2.0 Hz, 2H), 7.02-7.04 (m, 2H), 7.09-7.13 (m, 3H), 7.16-7.18 (m, 3H).
(4) Synthesis of (S,R)-2Ph-L11: (S,R)-2Ph-NH₂ (605 mg, 2.0 mmol, 1.0 equiv), 1-Cl (966 mg, 2.0 mmol, 1.0 equiv), Pd₂(dba)₃ (55 mg, 0.06 mmol, 3 mol %), JohnPhos (36 mg, 0.12 mmol, 6 mol %) and tBuONa (384 mg, 4.0 mmol, 2.0 equiv) were added to a dry tube equipped with a magnetic stir bar. The tube was evacuated and backfilled with nitrogen, the evacuation and back fill procedure was repeated for another twice. Then toluene (10 mL) was added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (20:1) as eluent to obtain the desired product (S,R)-2Ph-L11 as a yellow foamy solid 1.31 g in 87% yield.
(5) Synthesis of (S,R)-2Ph-LA11: (S,R)-2Ph-L11 (1.2 g, 1.60 mmol, 1.0 equiv), NH₄PF₆ (522 mg, 3.20 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (6 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (5:1) first, then dichloromethane/ethyl acetate (40:1) as eluent to obtain the desired product (S,R)-2Ph-LA11 as a yellow foamy solid 733 mg in 51% yield. ¹H NMR (500 MHz, DMSO-d₆): δ(ppm) 1.28 (s, 9H), 1.35 (s, 9H), 5.99 (d, J=3.0 Hz, 1H), 6.45 (d, J=3.0 Hz, 1H), 6.76-6.79 (m, 2H), 7.15-7.19 (m, 4H), 7.21 (dd, J=8.0, 2.0 Hz, 1H), 7.24-7.27 (m, 1H), 7.28-7.30 (m, 4H), 7.33-7.36 (m, 1H), 7.44 (d, J=8.0 Hz, 1H), 7.46-7.49 (m, 3H), 7.50 (d, J=2.5 Hz, 1H), 7.53 (d, J=8.5 Hz, 1H), 7.60 (t, J=2.0 Hz, 1H), 7.67 (d, J=1.5 Hz, 1H), 7.69 (t, J=8.5 Hz, 1H), 7.74 (d, J=8.0 Hz, 1H), 8.25 (d, J=7.5 Hz, 1H), 8.32 (d, J=8.5 Hz, 1H), 8.58 (d, J=5.5 Hz, 1H), 10.30 (s, 1H).
(6) Synthesis of (S,R)-2Ph-M-PtA11: (S,R)-2Ph-LA11 (300 mg, 0.33 mmol, 1.0 equiv), Pt(COD)Cl₂ (131 mg, 0.35 mmol, 1.05 equiv) and NaOAc (81 mg, 0.99 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (20 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (3:1) as eluent first, and then purified by slurry with ethyl acetate/petroleum ether to obtain the desired product (S,R)-2Ph-M-PtA11 as a pale yellow solid 30 mg in 10% yield. ¹H NMR (500 MHz, CDCl₃): δ(ppm) 1.29 (s, 9H), 1.42 (s, 9H), 5.90 (d, J=5.0 Hz, 2H), 6.00 (s, 1H), 6.18 (s, 1H), 6.84 (d, J=7.0 Hz, 3H), 6.97 (d, J=1.5 Hz, 1H), 7.19-7.26 (m, 8H), 7.33 (t, J=7.5 Hz, 1H), 7.38-7.48 (m, 3H), 7.54 (t, J=8.0 Hz, 1H), 7.77-7.82 (m, 2H), 7.98 (d, J=8.0 Hz, 1H), 8.07 (d, J=8.0 Hz, 2H), 10.14 (s, 1H).

### Example 23

The synthetic route for (R,S)-2Ph-P—PtA11 is as follows:

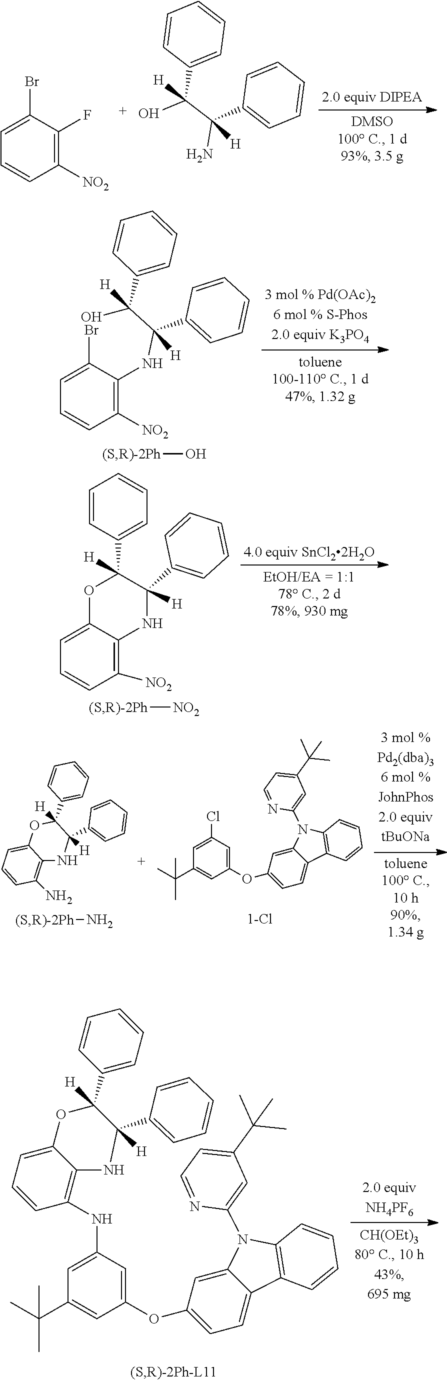

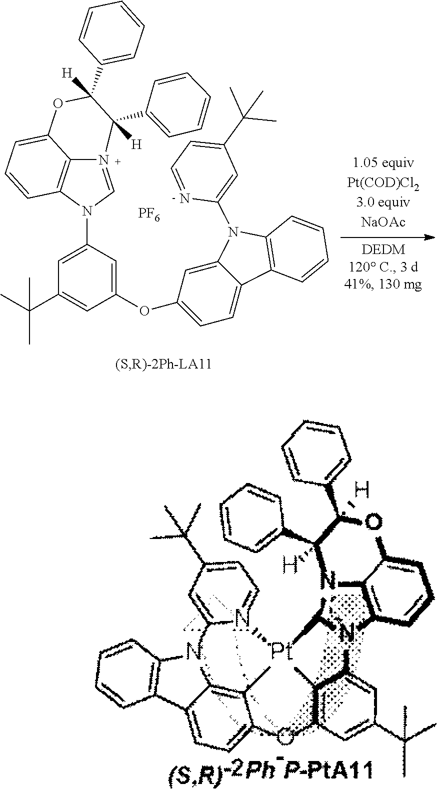

(1) Synthesis of (R,S)-2Ph-OH: 1-bromo-2-fluoro-3-nitrobenzene (2.0 g, 9.09 mmol, 1.0 equiv), (1R,2S)-2-amino-1,2-diphenylethan-1-ol (1.94 g, 9.09 mmol, 1.0 equiv) were added to a dry tube equipped with a magnetic stir bar. The tube was evacuated and backfilled with nitrogen, the evacuation and back fill procedure was repeated for another twice. Then N,N-Diisopropylethylamine (DIPEA) (2.35 g, 18.18 mmol, 2.0 equiv) and DMSO (15 mL) were added under a nitrogen atmosphere. The tube was sealed and the mixture was stirred at a temperature of 100° C. for 1 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate (15:1-5:1) as eluent to obtain the desired product (R,S)-2Ph-OH as a brown-yellow oily liquid 3.5 g in 93% yield. ¹H NMR (500 MHz, DMSO-d₆): δ(ppm) 4.92 (dd, J=9.5, 4.0 Hz, 1H), 5.14 (t, J=4.5 Hz, 1H), 6.13 (d, J=4.0 Hz, 1H), 6.78 (t, J=8.0 Hz, 1H), 6.81-6.84 (m, 2H), 7.00-7.08 (m, 5H), 7.10-7.18 (m, 4H), 7.72 (dd, J=8.5, 1.5 Hz, 1H), 7.81 (dd, J=8.0, 1.5 Hz, 1H)
(2) Synthesis of (R,S)-2Ph-NO₂: Pd(OAc)₂ (56 mg, 0.25 mmol, 3 mol %), SPhos (209 mg, 0.51 mmol, 6 mol %) and K₃PO₄ (3.6 g, 16.98 mmol, 2.0 equiv) were added to a dry tube equipped with a magnetic stir bar. The tube was evacuated and backfilled with nitrogen, the evacuation and back fill procedure was repeated for another twice. Then (R,S)-2Ph-OH (3.5 g, 8.49 mmol, 1.0 equiv) and toluene (40 mL) were added under a nitrogen atmosphere. The tube was sealed and the mixture was stirred at a temperature of 100-110° C. for 1 days, cooled down to ambient temperature. The solvent was removed under reduced pressure and the residue was purified through column chromatography on silica gel using petroleum ether/ethyl acetate/dichloromethane (10:1:1-6:1:1) as eluent to obtain the desired product (R,S)-2Ph-NO₂ as a yellow flake solid 1.32 g in 47% yield. ¹H NMR (500 MHz, DMSO-d₆): δ(ppm) 5.08 (dd, J=5.0, 3.0 Hz, 1H), 5.49 (d, J=3.0 Hz, 1H), 6.69 (dd, J=8.5, 7.5 Hz, 1H), 6.71-6.73 (m, 2H), 7.06-7.16 (m, 5H), 7.21-7.23 (m, 3H), 7.25-7.27 (m, 1H), 7.80 (dd, J=9.0, 1.5 Hz, 1H), 9.01 (d, J=4.5 Hz, 1H).
(3) Synthesis of (R,S)-2Ph-NH₂: (R,S)-2Ph-NO₂ (1.32 g, 3.97 mmol, 1.0 equiv), SnCl₂·2H₂O (3.58 g, 15.88 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (20 mL:20 mL) were added under a nitrogen atmosphere. The flask was sealed and the mixture was stirred at a temperature of 78° C. for 2 days, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/dichloromethane (2:1) as eluent to obtain the desired product (R,S)-2Ph-NH₂ as a yellow solid 930 mg in 78% yield. ¹H NMR (500 MHz, DMSO-d₆): δ(ppm) 4.67 (s, 2H), 4.76 (t, J=3.0 Hz, 1H), 5.32 (d, J=3.0 Hz, 1H), 5.44 (d, J=3.5 Hz, 1H), 6.20 (dd, J=8.0, 1.0 Hz, 1H), 6.28 (dd, J=8.0, 1.5 Hz, 1H), 6.41 (t, J=8.0 Hz, 1H), 6.87 (dd, J=8.0, 2.0 Hz, 2H), 7.02-7.04 (m, 2H), 7.08-7.13 (m, 3H), 7.16-7.18 (m, 3H).
(4) Synthesis of (R,S)-2Ph-L11: (R,S)-2Ph-NH₂ (605 mg, 2.0 mmol, 1.0 equiv), 1-Cl (966 mg, 2.0 mmol, 1.0 equiv), Pd₂(dba)₃ (55 mg, 0.06 mmol, 3 mol %), JohnPhos (36 mg, 0.12 mmol, 6 mol %) and tBuONa (384 mg, 4.0 mmol, 2.0 equiv) were added to a dry tube equipped with a magnetic stir bar. The tube was evacuated and backfilled with nitrogen, the evacuation and back fill procedure was repeated for another twice. Then toluene (10 mL) was added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (20:1) as eluent to obtain the desired product (R,S)-2Ph-L11 as a yellow foamy solid 1.34 g in 90% yield. ¹H NMR (500 MHz, DMSO-d₆): δ(ppm) 1.19 (s, 9H), 1.29 (s, 9H), 4.74 (t, J=3.5 Hz, 1H), 5.27 (d, J=3.0 Hz, 1H), 5.69 (d, J=3.5 Hz, 1H), 6.26 (t, J=2.0 Hz, 1H), 6.45-6.48 (m, 2H), 6.59 (dd, J=8.0, 1.0 Hz, 1H), 6.66 (t, J=1.4 Hz, 1H), 6.71 (dd, J=8.0, 1.0 Hz, 1H), 6.79 (dd, J=8.5, 1.5 Hz, 2H), 6.95 (dd, J=8.0, 2.0 Hz, 2H), 7.00-7.07 (m, 4H), 7.11-7.16 (m, 3H), 7.30-7.33 (m, 3H), 7.41-7.44 (m, 1H), 7.45 (dd, J=5.0, 1.5 Hz, 1H), 7.59 (d, J=1.0 Hz, 1H), 7.73 (d, J=8.5 Hz, 1H), 8.19 (d, J=8.0 Hz, 1H), 8.21 (d, J=8.5 Hz, 1H), 8.56 (d, J=5.5 Hz, 1H).
(5) Synthesis of (R,S)-2Ph-LA11: (R,S)-2Ph-L11 (1.34 g, 1.79 mmol, 1.0 equiv), NH₄PF₆ (584 mg, 3.58 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (6 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (5:1) first, then dichloromethane/ethyl acetate (40:1) as eluent to obtain the desired product (R,S)-2Ph-LA11 as a yellow foamy solid 695 mg in 43% yield. ¹H NMR (500 MHz, DMSO-d₆): δ(ppm) 1.28 (s, 9H), 1.35 (s, 9H), 5.99 (d, J=3.5 Hz, 1H), 6.45 (d, J=3.0 Hz, 1H), 6.76-6.78 (m, 2H), 7.15-7.17 (m, 4H), 7.20 (dd, J=8.5, 2.0 Hz, 1H), 7.24-7.27 (m, 1H), 7.28-7.31 (m, 4H), 7.33-7.36 (m, 1H), 7.44 (d, J=8.0 Hz, 1H), 7.45-7.49 (m, 3H), 7.50 (d, J=2.5 Hz, 1H), 7.53 (d, J=8.0 Hz, 1H), 7.60 (t, J=2.0 Hz, 1H), 7.67 (d, J=1.5 Hz, 1H), 7.69 (t, J=8.5 Hz, 1H), 7.74 (d, J=8.5 Hz, 1H), 8.24 (d, J=7.5 Hz, 1H), 8.32 (d, J=8.5 Hz, 1H), 8.57 (dd, J=5.5, 0.5 Hz, 1H), 10.30 (s, 1H).
(6) Synthesis of (R,S)-2Ph-P—PtA11: (R,S)-2Ph-LA11 (300 mg, 0.33 mmol, 1.0 equiv), Pt(COD)Cl₂ (131 mg, 0.35 mmol, 1.05 equiv) and NaOAc (81 mg, 0.99 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (20 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (3:1) as eluent first, and then purified by slurry with ethyl acetate/petroleum ether to obtain the desired product (R,S)-2Ph-P—PtA11 as a pale yellow solid 130 mg in 41% yield. ¹H NMR (500 MHz, CDCl₃): δ(ppm) 1.26 (s, 9H), 1.42 (s, 9H), 5.91 (s, 3H), 6.17 (s, 1H), 6.84 (s, 3H), 6.98 (d, J=1.0 Hz, 1H), 7.19-7.27 (m, 7H), 7.31-7.36 (m, 2H), 7.42-7.45 (m, 2H), 7.54 (t, J=8.0 Hz, 1H), 7.75 (d, J=6.6 Hz, 1H), 7.81 (d, J=8.0 Hz, 1H), 7.97 (d, J=8.5 Hz, 1H), 8.07 (d, J=8.0 Hz, 2H), 10.18 (s, 1H).

### Example 24

The synthetic route for (R,R)-M-PtA12 is as follows:

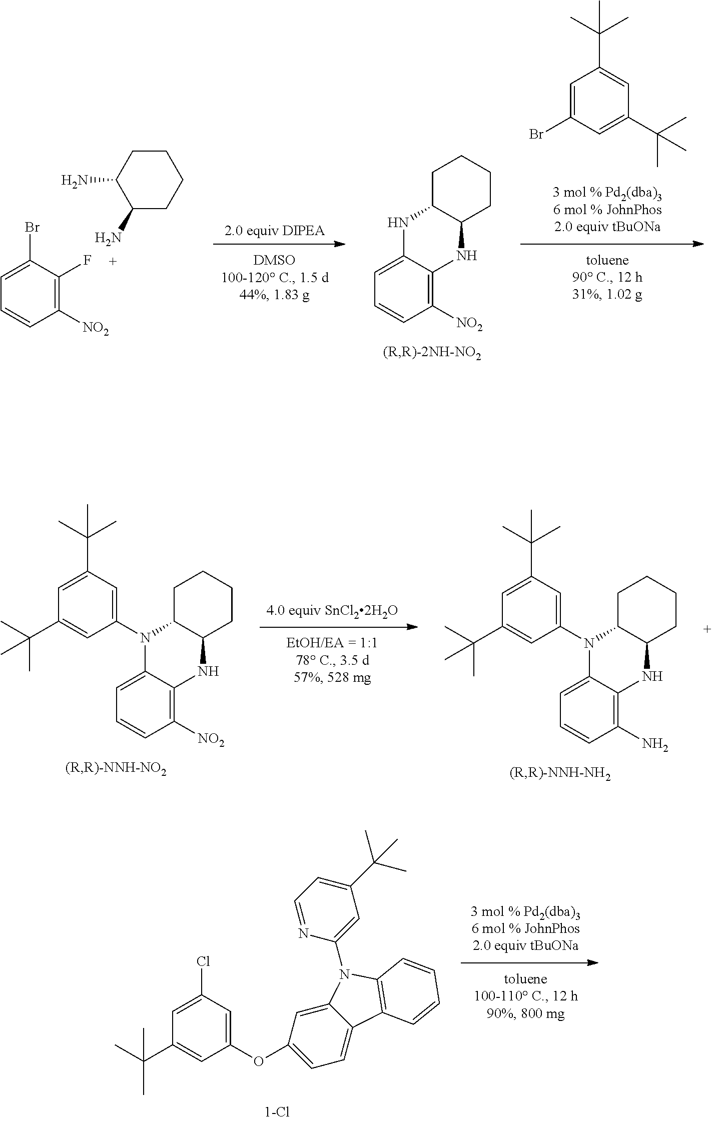

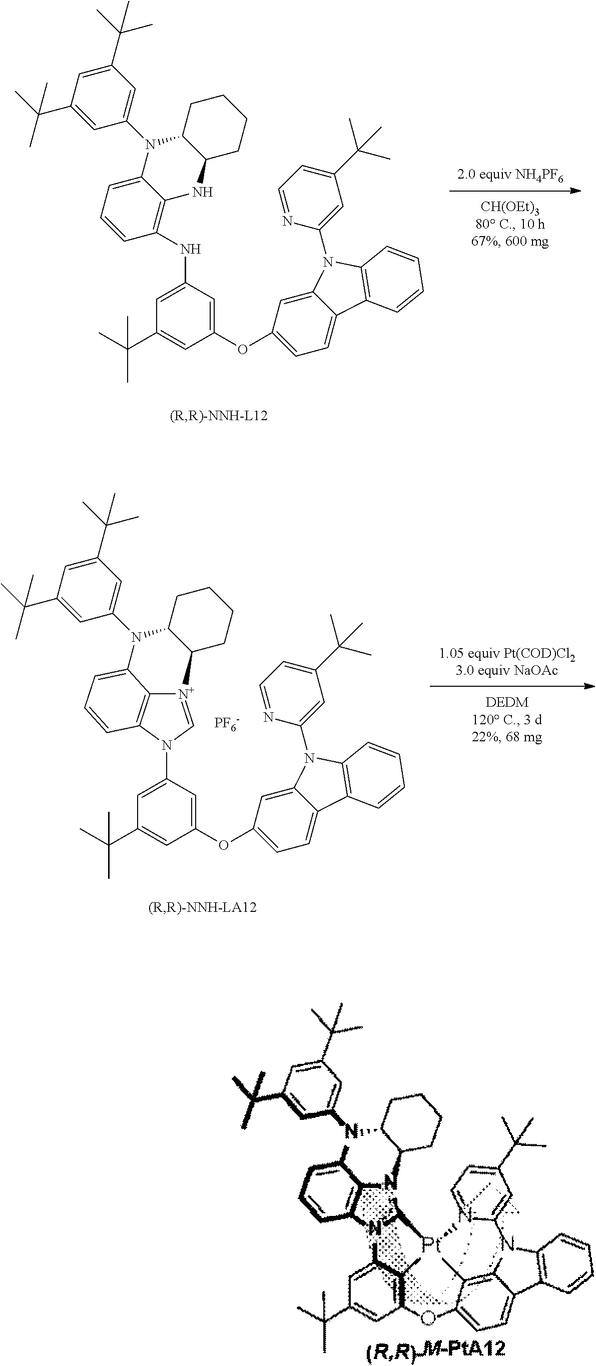

(1) Synthesis of (R,R)-2NH—NO₂: 1-bromo-2-fluoro-3-nitrobenzene (3.96 g, 18 mmol, 1.0 equiv), (1R,2R)-cyclohexane-1,2-diamine (2.26 g, 19.80 mmol, 1.1 equiv) were added to a dry tube equipped with a magnetic stir bar. The tube was evacuated and backfilled with nitrogen, the evacuation and back fill procedure was repeated for another twice. Then N,N-Diisopropylethylamine (DIPEA) (4.65 g, 36.00 mmol, 2.0 equiv) and DMSO (30 mL) were added under a nitrogen atmosphere. The tube was sealed and the mixture was stirred at a temperature of 100° C. for 1.5 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate (200:1-100:1) as eluent to obtain the desired product (R,R)-2NH—NO₂ as a red oily liquid 1.83 g in 44% yield. ¹H NMR (500 MHz, CDCl₃): δ(ppm) 1.30-1.41 (m, 2H), 1.42-1.48 (m, 2H), 1.81-1.88 (m, 2H), 1.93-1.96 (m, 1H), 2.04-2.07 (m, 1H), 2.83-2.88 (m, 1H), 3.12-3.17 (m, 1H), 3.96 (s, 1H), 6.46 (dd, J=8.5, 7.5 Hz, 1H), 6.61 (dt, J=7.0, 1.0 Hz, 1H), 7.55 (dd, J=9.0, 1.5 Hz, 1H), 7.87 (s, 1H).
(2) Synthesis of (R,R)—NNH—NO₂: (R,R)-2NH—NO₂ (1.83 g, 7.84 mmol, 1.0 equiv), 1-bromo-3,5-di-tert-butylbenzene (2.11 g, 7.84 mmol, 1.0 equiv), Pd₂(dba)₃ (140 mg, 0.47 mmol, 3 mol %), JohnPhos (220 mg, 0.24 mmol, 6 mol %) and tBuONa (1.51 g, 15.68 mmol, 2.0 equiv) were added to a dry tube equipped with a magnetic stir bar. The tube was evacuated and backfilled with nitrogen, the evacuation and back fill procedure was repeated for another twice. Then toluene (10 mL) was added under a nitrogen atmosphere. The tube was sealed and the mixture was stirred at a temperature of 90° C. for 12 hours, cooled down to ambient temperature. The solvent was removed under reduced pressure and the residue was purified through column chromatography on silica gel using petroleum ether/ethyl acetate (100:1-50:1) as eluent to obtain the desired product (R,R)—NNH—NO₂ as a red solid 1.02 g in 31% yield. ¹H NMR (500 MHz, CDCl₃): δ(ppm) 0.97-1.05 (m, 1H), 1.32 (s, 18H), 1.40-1.46 (m, 2H), 1.51-1.57 (m, 2H), 1.73 (d, J=13.5 Hz, 1H), 1.82 (d, J=11.5 Hz, 1H), 2.11-2.14 (m, 1H), 3.15-3.20 (m, 1H), 3.36-3.41 (m, 1H), 6.08 (dd, J=8.0, 1.0 Hz, 1H), 6.32 (dd, J=8.5, 7.5 Hz, 1H), 6.95 (d, J=2.0 Hz, 2H), 7.37 (t, J=2.0 Hz, 1H), 7.52 (dd, J=9.0, 1.5 Hz, 1H), 8.18 (s, 1H).
(3) Synthesis of (R,R)—NNH—NH₂: (R,R)—NNH—NO₂ (1.02 g, 2.42 mmol, 1.0 equiv), SnCl₂·2H₂O (2.18 g, 9.68 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (15 mL:15 mL) were added under a nitrogen atmosphere. The flask was sealed and the mixture was stirred at a temperature of 78° C. for 3.5 days, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/ethyl acetate (6:1) as eluent to obtain the desired product (R,R)—NNH—NH₂ as a white solid 528 mg in 57% yield. ¹H NMR (500 MHz, CDCl₃): δ(ppm) 0.98-1.06 (m, 1H), 1.30 (s, 18H), 1.37-1.45 (m, 2H), 1.56 (d, J=13.0 Hz, 1H), 1.68 (d, J=13.0 Hz, 1H), 1.77 (d, J=11.5 Hz, 1H), 2.08 (d, J=9.0 Hz, 1H), 2.78 (s, 3H), 3.12 (s, 3H), 5.65 (s, 1H), 6.17 (s, 1H), 6.41 (s, 1H), 6.99 (s, 2H), 7.31 (t, J=1.5 Hz, 1H).
(4) Synthesis of (R,R)—NNH-L12: (R,R)—NNH—NH₂ (392 mg, 1.0 mmol, 1.0 equiv), 1-Cl (531 mg, 1.1 mmol, 1.1 equiv), Pd₂(dba)₃ (27 mg, 0.03 mmol, 3 mol %), JohnPhos (18 mg, 0.06 mmol, 6 mol %) and tBuONa (192 mg, 2.0 mmol, 2.0 equiv) were added to a dry tube equipped with a magnetic stir bar. The tube was evacuated and backfilled with nitrogen, the evacuation and back fill procedure was repeated for another twice. Then toluene (10 mL) was added under a nitrogen atmosphere. The tube was sealed and the mixture was stirred at a temperature of 100-110° C. for 12 hours, cooled down to ambient temperature. The solvent was removed under reduced pressure and the residue was purified through column chromatography on silica gel using petroleum ether/ethyl acetate (30:1-20:1) as eluent to obtain the desired product (R,R)—NNH-L12 as a white solid 800 mg in 95% yield.
(5) Synthesis of (R,R)—NNH-LA12: (R,R)—NNH-L12 (800 mg, 0.95 mmol, 1.0 equiv), NH₄PF₆ (310 mg, 1.90 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (5 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (5:1) first, then dichloromethane/ethyl acetate (40:1) as eluent to obtain the desired product (R,R)—NNH-LA12 as a gray foamy solid 600 mg in 67% yield. ¹H NMR (500 MHz, DMSO-d₆): δ(ppm) 1.20-1.24 (m, 1H), 1.29 (s, 27H), 1.36 (s, 9H), 1.37-1.42 (m, 1H), 1.47-1.55 (m, 1H), 1.66-1.68 (m, 1H), 1.75-1.81 (m, 2H), 1.89 (d, J=12.0 Hz, 1H), 2.80-2.83 (m, 1H), 3.76-3.80 (m, 1H), 4.40-4.45 (m, 1H), 6.05 (d, J=8.0 Hz, 1H), 6.98 (d, J=8.5 Hz, 1H), 7.09 (s, 2H), 7.20 (dd, J=8.0, 2.0 Hz, 1H), 7.26 (t, J=2.0 Hz, 1H), 7.29 (t, J=8.5 Hz, 1H), 7.33-7.36 (m, 1H), 7.44-7.48 (m, 4H), 7.51 (d, J=2.0 Hz, 1H), 7.56 (t, J=2.0 Hz, 1H), 7.68 (d, J=1.0 Hz, 1H), 7.73 (d, J=8.0 Hz, 1H), 8.24 (d, J=7.5 Hz, 1H), 8.32 (d, J=8.5 Hz, 1H), 8.57 (d, J=5.0 Hz, 1H), 10.13 (s, 1H).
(6) Synthesis of (R,R)-M-PtA12: (R,R)—NNH-LA12 (300 mg, 0.33 mmol, 1.0 equiv), Pt(COD)Cl₂ (120 mg, 0.32 mmol, 1.05 equiv) and NaOAc (74 mg, 0.90 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (20 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent first, and then purified by slurry with ethyl acetate/petroleum ether to obtain the desired product (R,R)-M-PtA12 as a pale yellow solid 68 mg in 22% yield. ¹H NMR (500 MHz, DMSO-d₆): δ(ppm) 0.81-0.89 (m, 3H), 1.20-1.24 (m, 1H), 1.33 (s, 18H), 1.37 (s, 9H), 1.44 (s, 9H), 1.47-1.57 (m, 2H), 1.57-1.62 (m, 1H), 1.69-1.73 (m, 1H), 3.72-3.77 (m, 1H), 4.66-4.71 (m, 1H), 5.91 (d, J=8.0 Hz, 1H), 6.97 (d, J=1.5 Hz, 1H), 7.18-7.22 (m, 3H), 7.27 (d, J=8.5 Hz, 1H), 7.41 (t, J=7.0 Hz, 1H), 7.44-7.46 (m, 2H), 7.49-7.56 (m, 3H), 7.92 (d, J=8.0 Hz, 1H), 8.04 (d, J=8.0 Hz, 1H), 8.14 (d, J=2.0 Hz, 1H), 8.17 (dd, J=8.0, 1.0 Hz, 1H), 9.54 (d, J=6.5 Hz, 1H).

### Example 25

The synthetic route for (S,S)—P—PtA12 is as follows:

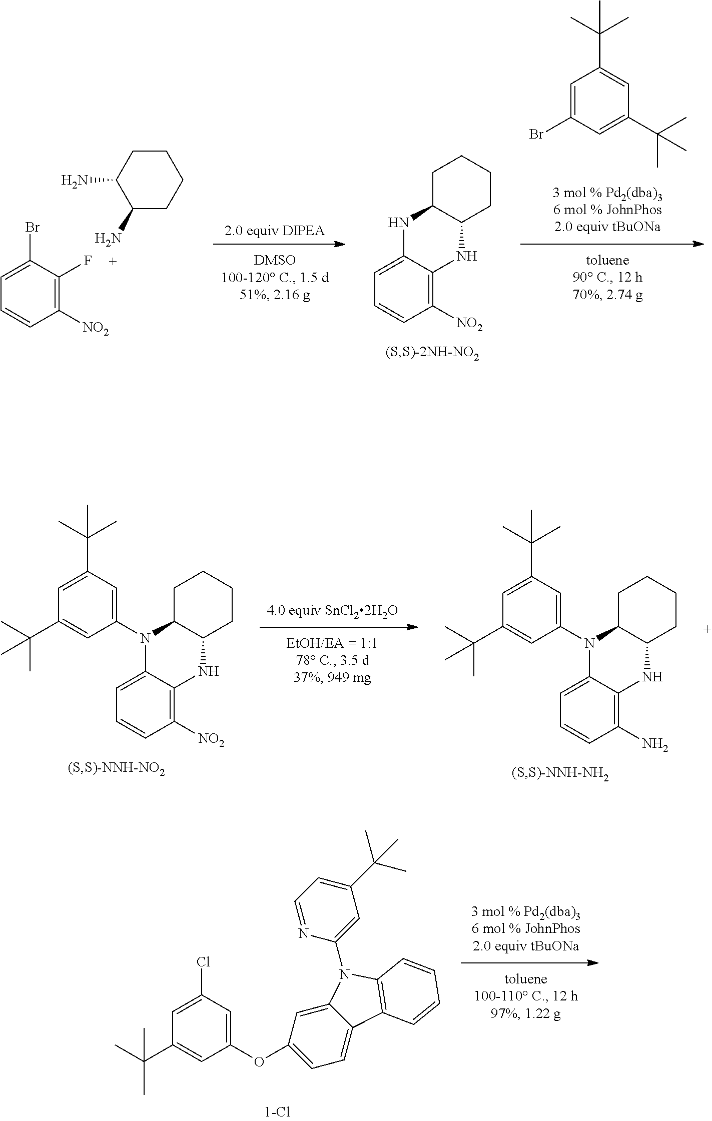

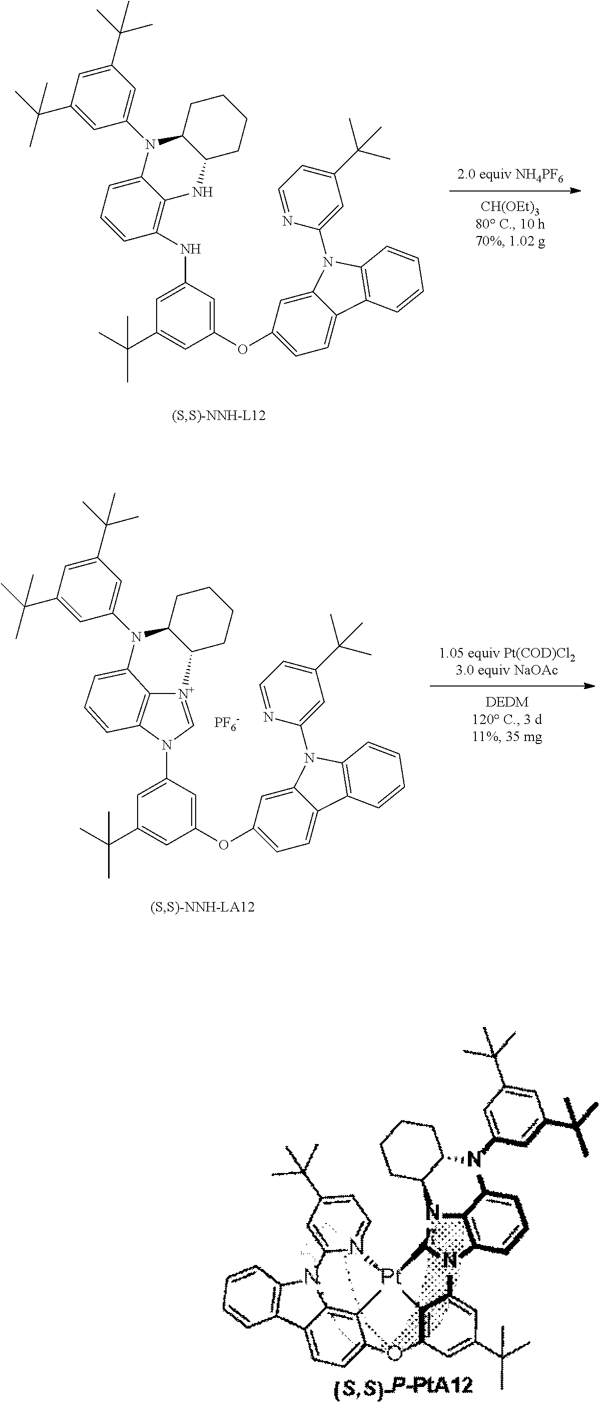

(1) Synthesis of (S,S)-2NH—NO₂: 1-bromo-2-fluoro-3-nitrobenzene (3.96 g, 18 mmol, 1.0 equiv), (1S,2S)-cyclohexane-1,2-diamine (2.26 g, 19.80 mmol, 1.1 equiv) were added to a dry tube equipped with a magnetic stir bar. The tube was evacuated and backfilled with nitrogen, the evacuation and back fill procedure was repeated for another twice. Then N,N-Diisopropylethylamine (DIPEA) (4.65 g, 36.00 mmol, 2.0 equiv) and DMSO (30 mL) were added under a nitrogen atmosphere. The tube was sealed and the mixture was stirred at a temperature of 100° C. for 1.5 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate (200:1-100:1) as eluent to obtain the desired product (S,S)-2NH—NO₂ as a red oily liquid 2.16 g in 51% yield. ¹H NMR (500 MHz, CDCl₃): δ(ppm) 1.27-1.38 (m, 2H), 1.40-1.48 (m, 2H), 1.82-1.8 (m, 2H), 1.93-1.96 (m, 1H), 2.04-2.07 (m, 1H), 2.84-2.88 (m, 1H), 3.13-3.17 (m, 1H), 3.98 (s, 1H), 6.46 (dd, J=8.5, 7.5 Hz, 1H), 6.61 (d, J=7.5 Hz, 1H), 7.55 (dd, J=9.0, 1.5 Hz, 1H), 7.87 (s, 1H).
(2) Synthesis of (S,S)—NNH—NO₂: (S,S)-2NH—NO₂ (2.16 g, 9.26 mmol, 1.0 equiv), 1-bromo-3,5-di-tert-butylbenzene (2.49 g, 9.26 mmol, 1.0 equiv), Pd₂(dba)₃ (256 mg, 18.52 mmol, 3 mol %), JohnPhos (167 mg, 0.56 mmol, 6 mol %) and tBuONa (1.78 g, 15.68 mmol, 2.0 equiv) were added to a dry tube equipped with a magnetic stir bar. The tube was evacuated and backfilled with nitrogen, the evacuation and back fill procedure was repeated for another twice. Then toluene (25 mL) was added under a nitrogen atmosphere. The tube was sealed and the mixture was stirred at a temperature of 90° C. for 12 hours, cooled down to ambient temperature. The solvent was removed under reduced pressure and the residue was purified through column chromatography on silica gel using petroleum ether/ethyl acetate (100:1-50:1) as eluent to obtain the desired product (S,S)—NNH—NO₂ as a red solid 2.74 g in 70% yield. ¹H NMR (500 MHz, CDCl₃): δ(ppm) 0.96-1.05 (m, 1H), 1.25-1.30 (m, 1H), 1.32 (s, 18H), 1.40-1.49 (m, 2H), 1.51-1.55 (m, 1H), 1.73 (d, J=14.5 Hz, 1H), 1.82 (d, J=11.5 Hz, 1H), 2.11-2.13 (m, 1H), 3.15-3.20 (m, 1H), 3.36-3.41 (m, 1H), 6.09 (d, J=8.0 Hz, 1H), 6.32 (dd, J=8.5, 7.5 Hz, 1H), 6.94 (d, J=1.5 Hz, 2H), 7.37 (t, J=1.5 Hz, 1H), 7.52 (dd, J=9.0, 1.5 Hz, 1H), 8.18 (s, 1H).
(3) Synthesis of (S,S)—NNH—NH₂: (S,S)—NNH—NO₂ (2.74 g, 6.50 mmol, 1.0 equiv), SnCl₂·2H₂O (5.87 g, 26.00 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (15 mL:15 mL) were added under a nitrogen atmosphere. The flask was sealed and the mixture was stirred at a temperature of 78° C. for 3.5 days, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/ethyl acetate (6:1) as eluent to obtain the desired product (S,S)—NNH—NH₂ as a white solid 949 mg in 37% yield. ¹H NMR (500 MHz, CDCl₃): δ(ppm) 0.98-1.06 (m, 1H), 1.30 (s, 18H), 1.37-1.45 (m, 2H), 1.55 (d, J=14.5 Hz, 1H), 1.65 (d, J=13.0 Hz, 1H), 1.77 (d, J=12.0 Hz, 1H), 2.07 (d, J=9.0 Hz, 1H), 2.55 (s, 3H), 3.13 (s, 2H), 5.65 (s, 1H), 6.17 (s, 1H), 6.41 (s, 1H), 6.99 (s, 2H), 7.31 (t, J=1.5 Hz, 1H).
(4) Synthesis of (S,S)—NNH-L12: (S,S)—NNH—NH₂ (587 mg, 1.5 mmol, 1.0 equiv), 1-Cl (797 mg, 1.65 mmol, 1.1 equiv), Pd₂(dba)₃ (241 mg, 0.045 mmol, 3 mol %), JohnPhos (27 mg, 0.09 mmol, 6 mol %) and tBuONa (288 mg, 3.0 mmol, 2.0 equiv) were added to a dry tube equipped with a magnetic stir bar. The tube was evacuated and backfilled with nitrogen, the evacuation and back fill procedure was repeated for another twice. Then toluene (10 mL) was added under a nitrogen atmosphere. The tube was sealed and the mixture was stirred at a temperature of 100-110° C. for 12 hours, cooled down to ambient temperature. The solvent was removed under reduced pressure and the residue was purified through column chromatography on silica gel using petroleum ether/ethyl acetate (30:1-20:1) as eluent to obtain the desired product (S,S)—NNH-L12 as a white solid 1.22 g in 97% yield.
(5) Synthesis of (S,S)—NNH-LA12: (S,S)—NNH-L12 (1.22 g, 1.46 mmol, 1.0 equiv), NH₄PF₆ (476 mg, 2.92 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (8 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (5:1) first, then dichloromethane/ethyl acetate (40:1) as eluent to obtain the desired product (S,S)—NNH-LA12 as a gray foamy solid 1.02 g in 70% yield. ¹H NMR (500 MHz, DMSO-d₆): δ(ppm) 1.23 (s, 1H), 1.29 (s, 27H), 1.36 (s, 9H), 1.37-1.42 (m, 1H), 1.48-1.55 (m, 1H), 1.65-1.67 (m, 1H), 1.75-1.81 (m, 2H), 1.88 (d, J=11.5 Hz, 1H), 2.80-2.82 (m, 1H), 3.76-3.81 (m, 1H), 4.40-4.45 (m, 1H), 6.05 (d, J=8.0 Hz, 1H), 6.98 (d, J=8.5 Hz, 1H), 7.09 (s, 2H), 7.20 (dd, J=8.5, 2.0 Hz, 1H), 7.25 (t, J=2.0 Hz, 1H), 7.29 (t, J=8.0 Hz, 1H), 7.33-7.36 (m, 1H), 7.44-7.48 (m, 4H), 7.50 (d, J=2.0 Hz, 1H), 7.55 (t, J=2.0 Hz, 1H), 7.67 (d, J=1.0 Hz, 1H), 7.75 (d, J=8.5 Hz, 1H), 8.24 (d, J=7.5 Hz, 1H), 8.32 (d, J=8.5 Hz, 1H), 8.57-8.58 (m, 1H), 10.12 (s, 1H).
(6) Synthesis of (S,S)—P—PtA12: (S,S)—NNH-LA12 (300 mg, 0.33 mmol, 1.0 equiv), Pt(COD)Cl₂ (120 mg, 0.32 mmol, 1.05 equiv) and NaOAc (74 mg, 0.90 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (20 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent first, and then purified by slurry with ethyl acetate/petroleum ether to obtain the desired product (S,S)—P—PtA12 as a pale yellow solid 35 mg in 11% yield. ¹H NMR (500 MHz, DMSO-d₆): δ(ppm) 0.81-0.89 (m, 3H), 1.20-1.26 (m, 1H), 1.33 (s, 18H), 1.37 (s, 9H), 1.44 (s, 9H), 1.47-1.57 (m, 2H), 1.58-1.62 (m, 1H), 1.70-1.73 (m, 1H), 3.72-3.76 (m, 1H), 4.66-4.71 (m, 1H), 5.91 (d, J=8.0 Hz, 1H), 6.97 (d, J=1.5 Hz, 1H), 7.18-7.22 (m, 3H), 7.27 (d, J=8.5 Hz, 1H), 7.40 (t, J=8.0 Hz, 1H), 7.44-7.46 (m, 2H), 7.49-7.57 (m, 1H), 7.91 (d, J=8.0 Hz, 1H), 8.04 (d, J=8.0 Hz, 1H), 8.14 (d, J=1.5 Hz, 1H), 8.17 (d, J=7.0 Hz, 1H), 9.54 (d, J=6.0 Hz, 1H).

### Example 26

The synthetic route for (R,S)-M-PtB1 is as follows:

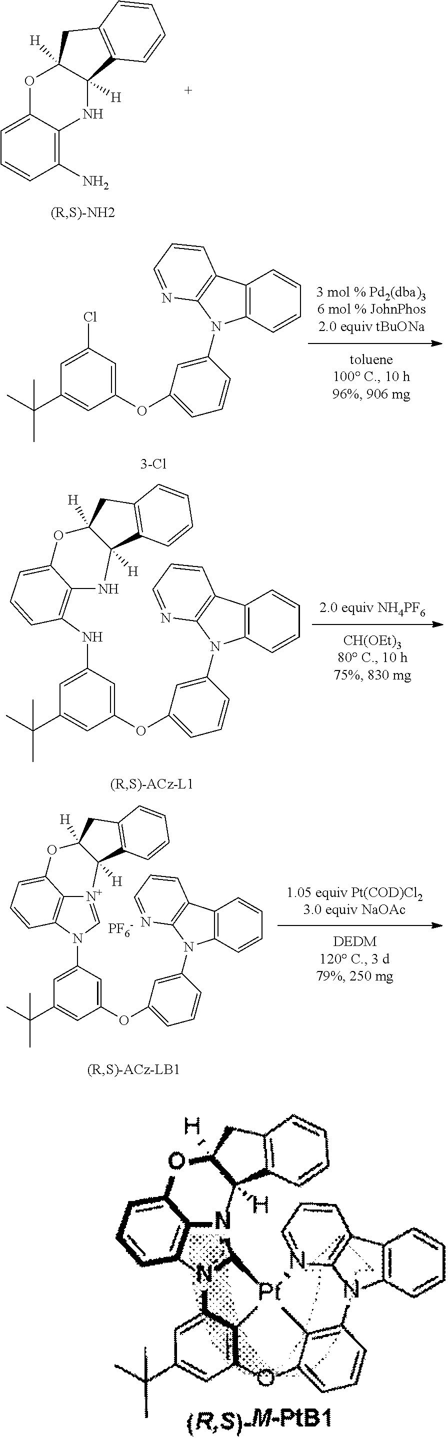

(1) Synthesis of (R,S)-ACz-L1: (R,S)—Nz₂ (587 mg, 1.5 mmol, 1.0 equiv), 3-Cl (357 mg, 1.5 mmol, 1.1 equiv), Pd₂(dba)₃ (41 mg, 0.045 mmol, 3 mol %), JohnPhos (27 mg, 0.09 mmol, 6 mol %) and tBuONa (288 mg, 3.0 mmol, 2.0 equiv) were added to a dry tube equipped with a magnetic stir bar. The tube was evacuated and backfilled with nitrogen, the evacuation and back fill procedure was repeated for another twice. Then toluene (10 mL) was added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (20:1-10:1) as eluent to obtain the desired product (R,S)-ACz-L1 as a yellow foamy solid 906 mg in 96% yield.
(2) Synthesis of (R,S)-ACz-LB1: (R,S)-ACz-L1 (896 mg, 1.42 mmol, 1.0 equiv), NH₄PF₆ (463 mg, 2.84 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (8 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (5:1) first, then dichloromethane/ethyl acetate (40:1) as eluent to obtain the desired product (R,S)-ACz-LB1 as a brown foamy solid 830 mg in 75% yield. ¹H NMR (500 MHz, DMSO-d₆): δ(ppm) 1.40 (s, 9H), 3.32 (dd, J=17.0, 2.5 Hz, 1H), 3.57 (dd, J=16.5, 4.5 Hz, 1H), 5.44-5.47 (m, 1H), 6.38 (d, J=4.0 Hz, 1H), 7.19 (d, J=8.0 Hz, 1H), 7.30-7.35 (m, 3H), 7.37 (dd, J=9.0, 2.0 Hz, 1H), 7.39-7.43 (m, 2H), 7.45-7.49 (m, 4H), 7.50-7.56 (m, 3H), 7.57 (t, J=2.0 Hz, 1H), 7.62 (d, J=7.5 Hz, 1H), 7.68 (t, J=1.5 Hz, 1H), 7.73 (t, J=8.0 Hz, 1H), 8.30 (d, J=7.5 Hz, 1H), 8.38 (dd, J=5.5, 2.0 Hz, 1H), 8.65 (dd, J=8.0, 1.5 Hz, 1H), 10.49 (s, 1H).
(3) Synthesis of (R,S)-M-PtB1: (R,S)-ACz-LB1 (300 mg, 0.38 mmol, 1.0 equiv), Pt(COD)Cl₂ (150 mg, 0.40 mmol, 1.05 equiv) and NaOAc (94 mg, 1.14 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (23 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (R,S)-M-PtB1 as a pale yellow solid 250 mg in 79% yield. ¹H NMR (500 MHz, DMSO-d₆): δ(ppm) 1.47 (s, 9H), 3.25 (d, J=17.0 Hz, 1H), 3.31-3.34 (m, 1H), 5.24 (t, J=3.5 Hz, 1H), 6.13 (t, J=7.0 Hz, 1H), 6.26 (d, J=2.0 Hz, 1H), 6.81 (d, J=8.0 Hz, 1H), 6.89 (t, J=7.5 Hz, 1H), 7.03 (d, J=7.5 Hz, 1H), 7.07 (d, J=1.5 Hz, 1H), 7.10-7.12 (m, 1H), 7.19 (dd, J=8.0, 1.5 Hz, 1H), 7.22 (t, J=7.5 Hz, 2H), 7.32 (t, J=8.0 Hz, 1H), 7.44 (t, J=8.0 Hz, 1H), 7.52-7.53 (m, 2H), 7.64-7.47 (m, 1H), 7.70 (d, J=8.5 Hz, 1H), 8.14 (d, J=8.5 Hz, 1H), 8.21 (d, J=7.5 Hz, 1H), 8.51 (dd, J=7.5, 1.5 Hz, 1H), 9.52 (dd, J=5.0, 1.0 Hz, 1H).

### Example 27

The synthetic route for (S,R)—P—PtB1 is as follows:

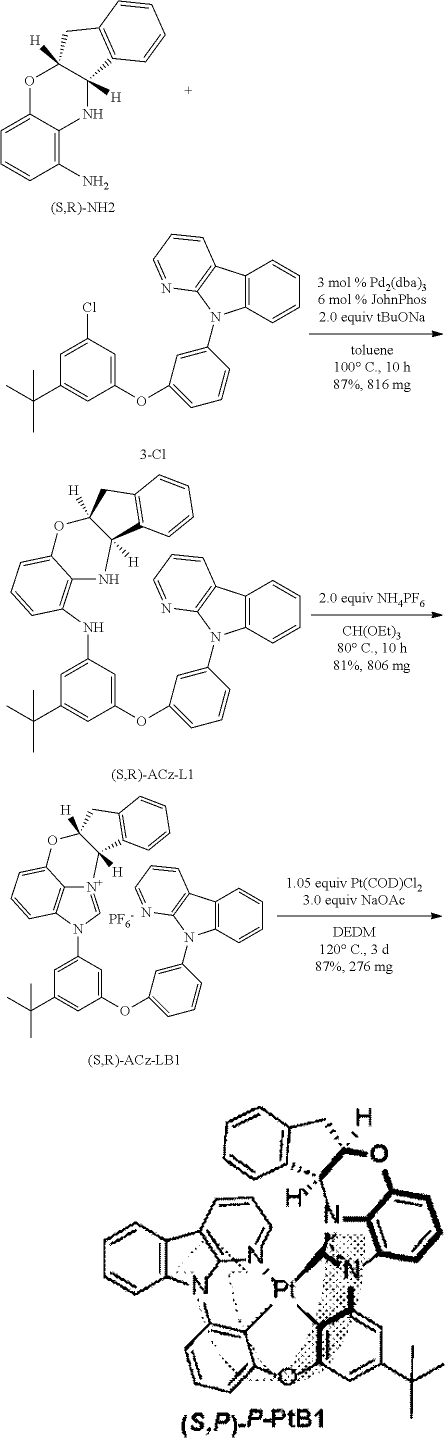

(1) Synthesis of (S,R)-ACz-L1: (S,R)—NH₂ (357 mg, 1.5 mmol, 1.0 equiv), 3-Cl (357 mg, 1.5 mmol, 1.1 equiv), Pd₂(dba)₃ (41 mg, 0.045 mmol, 3 mol %), JohnPhos (27 mg, 0.09 mmol, 6 mol %) and tBuONa (288 mg, 3.0 mmol, 2.0 equiv) were added to a dry tube equipped with a magnetic stir bar. The tube was evacuated and backfilled with nitrogen, the evacuation and back fill procedure was repeated for another twice. Then toluene (10 mL) was added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (20:1-10:1) as eluent to obtain the desired product (S,R)-A Cz-L1 as a yellow foamy solid 816 mg in 87% yield.
(2) Synthesis of (S,R)-ACz-LB1: (S,R)-A Cz-L1 (796 mg, 1.27 mmol, 1.0 equiv), NH₄PF₅ (414 mg, 2.54 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (4 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (5:1) first, then dichloromethane/ethyl acetate (40:1) as eluent to obtain the desired product (S,R)-ACz-LB1 as a brown foamy solid 806 mg in 81% yield. ¹H NMR (500 MHz, DMSO-d₆): δ(ppm) 1.40 (s, 9H), 3.33 (dd, J=16.5, 2.5 Hz, 1H), 3.57 (dd, J=16.5, 4.5 Hz, 1H), 5.44-5.47 (m, 1H), 6.38 (d, J=4.0 Hz, 1H), 7.19 (d, J=7.5 Hz, 1H), 7.30-7.35 (m, 3H), 7.36-7.38 (m, 1H), 7.39-7.43 (m, 2H), 7.45-7.50 (m, 4H), 7.50-7.55 (m, 3H), 7.57 (t, J=1.5 Hz, 1H), 7.62 (d, J=7.5 Hz, 1H), 7.68 (t, J=1.5 Hz, 1H), 7.73 (t, J=8.0 Hz, 1H), 8.30 (d, J=7.5 Hz, 1H), 8.38 (dd, J=5.0, 1.5 Hz, 1H), 8.65 (dd, J=7.5, 1.5 Hz, 1H), 10.49 (s, 1H).
(3) Synthesis of (S,R)—P—PtB1: (S,R)-ACz-LB1 (300 mg, 0.38 mmol, 1.0 equiv), Pt(COD)Cl₂ (150 mg, 0.40 mmol, 1.05 equiv) and NaOAc (94 mg, 1.14 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (23 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (S,R)—P—PtB1 as a pale yellow solid 276 mg in 87% yield. ¹H NMR (500 MHz, DMSO-d₆): δ(ppm) 1.47 (s, 9H), 3.25 (d, J=17.0 Hz, 1H), 3.31-3.34 (m, 1H), 5.24 (t, J=4.0 Hz, 1H), 6.13 (t, J=7.0 Hz, 1H), 6.26 (d, J=2.5 Hz, 1H), 6.81 (d, J=8.0 Hz, 1H), 6.89 (t, J=7.5 Hz, 1H), 7.03 (d, J=7.6 Hz, 1H), 7.07 (d, J=1.5 Hz, 1H), 7.10-7.15 (m, 1H), 7.19 (dd, J=8.0, 1.5 Hz, 1H), 7.22 (t, J=7.5 Hz, 2H), 7.32 (t, J=8.0 Hz, 1H), 7.44 (t, J=8.0 Hz, 1H), 7.52-7.53 (m, 2H), 7.64-7.67 (m, 1H), 7.70 (d, J=8.5 Hz, 1H), 8.14 (d, J=8.5 Hz, 1H), 8.21 (d, J=7.5 Hz, 1H), 8.51 (dd, J=7.5, 1.5 Hz, 1H), 9.52 (dd, J=5.5, 1.0 Hz, 1H).

### Example 28

The synthetic route for (R,S)-M-PtC1 is as follows:

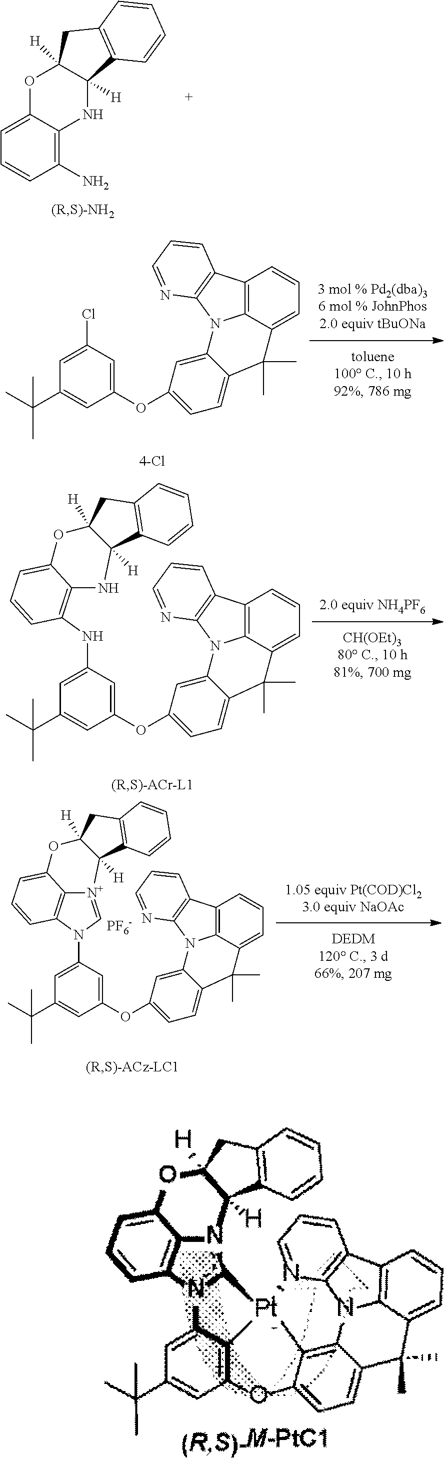

(1) Synthesis of (R,S)-Acr-L1: (R,S)—NH₂ (306 mg, 1.28 mmol, 1.0 equiv), 4-Cl (600 mg, 1.28 mmol, 1.0 equiv), Pd₂(dba)₃ (35 mg, 0.039 mmol, 3 mol %), JohnPhos (23 mg, 0.077 mmol, 6 mol %) and tBuONa (247 mg, 2.57 mmol, 2.0 equiv) were added to a dry tube equipped with a magnetic stir bar. The tube was evacuated and backfilled with nitrogen, the evacuation and back fill procedure was repeated for another twice. Then toluene (10 mL) was added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (20:1-10:1) as eluent to obtain the desired product (R,S)-Acr-L1 as a dark green, foamy solid 786 mg in 92% yield. ¹H NMR (500 MHz, DMSO-d₆): δ(ppm) 1.28 (s, 9H), 1.70 (d, J=4.5 Hz, 6H), 2.96 (d, J=16.5 Hz, 1H), 3.11 (dd, J=16.0, 3.5 Hz, 1H), 4.36 (t, J=3.5 Hz, 1H), 4.83 (t, J=5.0 Hz, 1H), 5.90 (d, J=6.0 Hz, 1H), 6.20 (t, J=22.5 Hz, 1H), 6.23 (t, J=8.0 Hz, 1H), 6.32 (dd, J=8.0, 1.0 Hz, 1H), 6.58 (t, J=1.5 Hz, 1H), 6.66 (dd, J=7.59, 1.0 Hz, 1H), 6.73 (t, J=1.5 Hz, 1H), 6.84 (dd, J=9.0, 3.0 Hz, 1H), 7.09-7.14 (m, 2H), 7.20-7.22 (m, 1H), 7.30-7.32 (m, 1H), 7.35 (s, 1H), 7.38 (dd, J=7.5, 5.0 Hz, 1H), 7.43 (t, J=8.0 Hz, 1H), 7.67 (dd, J=8.0, 1.0 Hz, 1H), 7.70 (d, J=9.0 Hz, 1H), 8.06 (dd, J=8.0, 1.0 Hz, 1H), 8.54 (dd, J=5.0, 1.5 Hz, 1H), 8.64 (dd, J=7.5, 1.5 Hz, 1H), 9.28 (d, J=3.0 Hz, 1H).
(2) Synthesis of (R,S)-Acr-LC1: (R,S)-Acr-L1 (700 mg, 1.05 mmol, 1.0 equiv), NH₄PF₆ (341 mg, 2.09 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (7 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (5:1) first, then dichloromethane/ethyl acetate (40:1) as eluent to obtain the desired product (R,S)-Acr-LC1 as a brown foamy solid 700 mg in 81% yield. ¹H NMR (500 MHz, DMSO-d₆): δ(ppm) 1.42 (s, 9H), 1.72 (s, 6H), 3.30 (dd, J=17.0, 2.5 Hz, 1H), 3.54 (dd, J=16.5, 5.0 Hz, 1H), 5.41-5.43 (m, 1H), 6.36 (d, J=4.0 Hz, 1H), 7.05 (dd, J=8.5, 5.5 Hz, 1H), 7.14 (dd, J=7.5, 0.5 Hz, 1H), 7.30 (t, J=7.5 Hz, 1H), 7.43-7.45 (m, 7H), 7.59-7.61 (m, 2H), 7.68-7.70 (m, 2H), 7.81 (d, J=9.0 Hz, 1H), 8.08 (dd, J=7.5, 1.0 Hz, 1H), 8.54 (dd, J=5.0, 1.5 Hz, 1H), 8.66 (dd, J=8.0, 2.0 Hz, 1H), 9.40 (d, J=2.5 Hz, 1H), 10.49 (s, 1H).
(3) Synthesis of (R,S)-M-PtC1: (R,S)-Acr-LC1 (300 mg, 0.36 mmol, 1.0 equiv), Pt(COD)Cl₂ (143 mg, 0.38 mmol, 1.05 equiv) and NaOAc (90 mg, 1.09 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (23 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (R,S)-M-PtC1 as a pale yellow solid 207 mg in 66% yield. ¹H NMR (500 MHz, DMSO-d₆): δ(ppm) 1.46 (s, 9H), 1.58 (s, 3H), 1.89 (s, 3H), 3.28 (d, J=16.5 Hz, 1H), 3.34-3.36 (m, 1H), 5.48 (t, J=3.0 Hz, 1H), 6.36 (d, J=3.0 Hz, 1H), 6.73 (t, J=7.5 Hz, 1H), 6.84 (d, J=8.0 Hz, 1H), 7.00 (d, J=1.5 Hz, 1H), 7.02 (t, J=7.5 Hz, 1H), 7.21 (d, J=8.5 Hz, 1H), 7.25 (d, J=7.5 Hz, 1H), 7.29 (t, J=5.5 Hz, 1H), 7.35 (t, J=8.0 Hz, 1H), 7.47-7.51 (m, 2H), 7.54 (d, J=1.5 Hz, 1H), 7.57 (d, J=7.5 Hz, 1H), 7.73-7.76 (m, 2H), 8.07 (dd, J=7.5, 1.0 Hz, 1H), 8.80 (dd, J=7.5, 1.5 Hz, 1H), 9.66 (dd, J=5.5, 0.5 Hz, 1H).

### Example 29

The synthetic route for (S,R)—P—PtC1 is as follows:

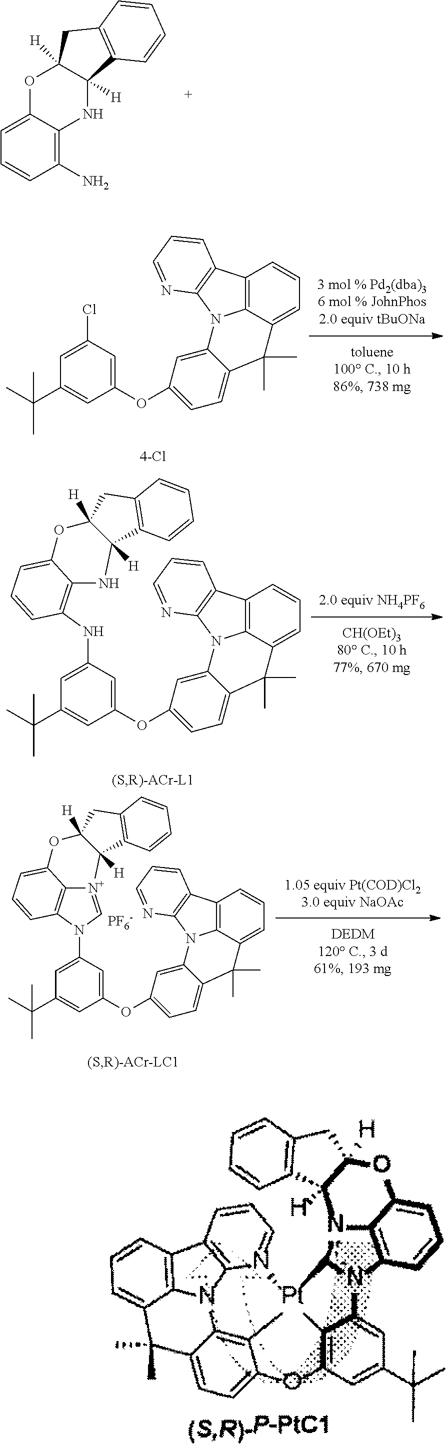

(1) Synthesis of (S,R)-Acr-L1: (S,R)—NH₂ (306 mg, 1.28 mmol, 1.0 equiv), 4-Cl (600 mg, 1.28 mmol, 1.0 equiv), Pd₂(dba)₃ (35 mg, 0.039 mmol, 3 mol %), JohnPhos (23 mg, 0.077 mmol, 6 mol %) and tBuONa (247 mg, 2.57 mmol, 2.0 equiv) were added to a dry tube equipped with a magnetic stir bar. The tube was evacuated and backfilled with nitrogen, the evacuation and back fill procedure was repeated for another twice. Then toluene (10 mL) was added under a nitrogen atmosphere. The tube was sealed and the mixture was stirred at a temperature of 100° C. for 10 hours, cooled down to ambient temperature. The solvent was removed under reduced pressure and the residue was purified through column chromatography on silica gel using petroleum ether/ethyl acetate (20:1-10:1) as eluent to obtain the desired product (S,R)-Acr-L1 as a dark green, foamy solid 738 mg in 86% yield. ¹H NMR (500 MHz, DMSO-d₆): δ(ppm) 1.28 (s, 9H), 1.71 (d, J=4.5 Hz, 6H), 2.96 (d, J=16.5 Hz, 1H), 3.11 (dd, J=16.0, 4.0 Hz, 1H), 4.36 (t, J=4.0 Hz, 1H), 4.83 (t, J=4.5 Hz, 1H), 5.90 (d, J=6.0 Hz, 1H), 6.20 (t, J=2.0 Hz, 1H), 6.23 (t, J=8.0 Hz, 1H), 6.32 (dd, J=8.0, 1.0 Hz, 1H), 6.58 (t, J=2.0 Hz, 1H), 6.66 (dd, J=8.0, 1.0 Hz, 1H), 6.73 (t, J=1.5 Hz, 1H), 6.84 (dd, J=8.5, 2.5 Hz, 1H), 7.09-7.14 (m, 2H), 7.21-7.22 (m, 1H), 7.30-7.32 (m, 1H), 7.35 (s, 1H), 7.39 (dd, J=8.0, 5.0 Hz, 1H), 7.43 (t, J=7.5 Hz, 1H), 7.68 (dd, J=7.5, 0.5 Hz, 1H), 7.70 (d, J=9.0 Hz, 1H), 8.07 (dd, J=8.0, 1.0 Hz, 1H), 8.54 (dd, J=5.0, 1.5 Hz, 1H), 8.64 (dd, J=7.5, 1.5 Hz, 1H), 9.28 (d, J=2.5 Hz, 1H).
(2) Synthesis of (S,R)-Acr-LC1: (S,R)-Acr-L1 (700 mg, 1.05 mmol, 1.0 equiv), NH₄PF₆ (341 mg, 2.09 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (7 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (5:1) first, then dichloromethane/ethyl acetate (40:1) as eluent to obtain the desired product (S,R)-Acr-LC1 as a brown foamy solid 670 mg in 77% yield. ¹H NMR (500 MHz, DMSO-d₆): δ(ppm) 1.41 (s, 9H), 1.72 (s, 6H), 3.30 (dd, J=17.0, 2.0 Hz, 1H), 3.54 (dd, J=17.0, 5.0 Hz, 1H), 5.40-5.43 (m, 1H), 6.35 (d, J=4.0 Hz, 1H), 7.04 (dd, J=9.0, 3.0 Hz, 1H), 7.14 (dd, J=8.0, 0.5 Hz, 1H), 7.30 (t, J=7.5 Hz, 1H), 7.34-7.45 (m, 7H), 7.59-7.61 (m, 2H), 7.68-7.70 (m, 2H), 7.81 (d, J=9.0 Hz, 1H), 8.08 (dd, J=7.5, 0.5 Hz, 1H), 8.54 (dd, J=5.0, 2.0 Hz, 1H), 8.66 (dd, J=8.0, 1.5 Hz, 1H), 9.40 (d, J=3.0 Hz, 1H), 10.49 (s, 1H).
(3) Synthesis of (S,R)—P—PtC1: (S,R)-Acr-LC1 (300 mg, 0.36 mmol, 1.0 equiv), Pt(COD)Cl₂ (143 mg, 0.38 mmol, 1.05 equiv) and NaOAc (90 mg, 1.09 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (23 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (S,R)—P—PtC1 as a pale yellow solid 193 mg in 61% yield. ¹H NMR (500 MHz, DMSO-d₆): δ(ppm) 1.46 (s, 9H), 1.57 (s, 3H), 1.89 (s, 3H), 3.28 (d, J=17.0 Hz, 1H), 3.36-3.42 (m, 1H), 5.48 (t, J=3.5 Hz, 1H), 6.36 (d, J=3.5 Hz, 1H), 6.72 (t, J=7.5 Hz, 1H), 6.84 (d, J=8.0 Hz, 1H), 7.00 (d, J=1.5 Hz, 1H), 7.02 (t, J=7.5 Hz, 1H), 7.21 (d, J=8.5 Hz, 1H), 7.25 (d, J=7.5 Hz, 1H), 7.29 (t, J=5.5 Hz, 1H), 7.35 (t, J=8.0 Hz, 1H), 7.47-7.58 (m, 2H), 7.54 (d, J=1.5 Hz, 1H), 7.57 (d, J=7.5 Hz, 1H), 7.75 (t, J=8.5 Hz, 2H), 8.07 (dd, J=7.5, 1.0 Hz, 1H), 8.80 (dd, J=8.0, 1.5 Hz, 1H), 9.65 (d, J=6.5 Hz, 1H).

### Example 30

The synthetic route for (S)-M-PtD1 is as follows:

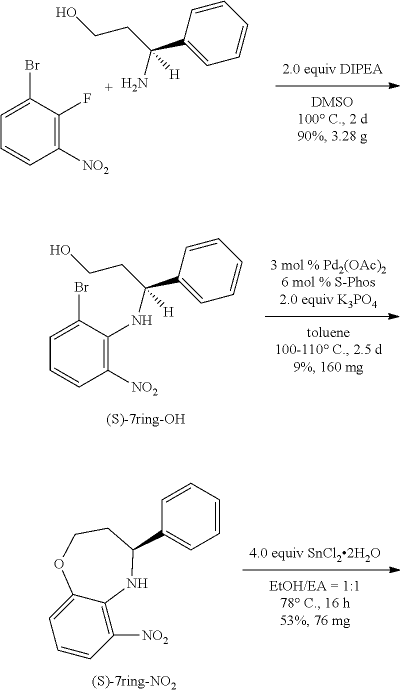 [SMILES: O=[N+]([O-])c1cccc(Br)c1F ; N[C@@H](CCO)c1ccccc1 ; O=[N+]([O-])c1cccc(Br)c1N[C@@H](CCO)c1ccccc1 ; O=[N+]([O-])c1cccc2c1N[C@H](c1ccccc1)CCO2]

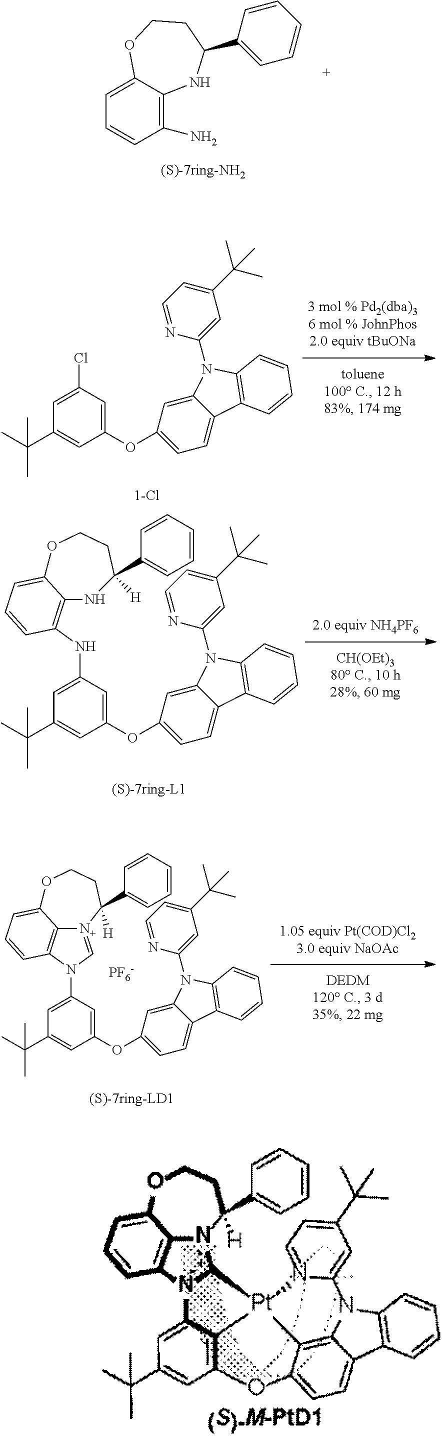

m
(1) Synthesis of (S)-7ring-OH: 1-bromo-2-fluoro-3-nitrobenzene (2.2 g, 10.0 mmol, 1.0 equiv), (S)-3-amino-3-phenylpropan-1-ol (1.64 g, 11.0 mmol, 1.0 equiv) were added to a dry tube equipped with a magnetic stir bar. The tube was evacuated and backfilled with nitrogen, the evacuation and back fill procedure was repeated for another twice. Then N,N-Diisopropylethylamine (DIPEA) (2.58 g, 20 mmol, 2.0 equiv) and DMSO (20 mL) were added under a nitrogen atmosphere. The tube was sealed and the mixture was stirred at a temperature of 100° C. for 2 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate (10:1-5:1) as eluent to obtain the desired product (S)-7ring-OH as a yellow oily liquid 3.28 g in 90% yield. ¹H NMR (500 MHz, CDCl₃): δ(ppm) 1.65 (s, 1H), 2.05-2.10 (m, 1H), 2.23-2.30 (m, 1H), 3.81-3.88 (m, 2H), 5.00-5.05 (m, 1H), 6.61 (d, J=10.0 Hz, 1H), 6.73 (t, J=8.5 Hz, 1H), 7.06-7.09 (m, 2H), 7.16-7.23 (m, 3H), 7.67 (dd, J=8.0, 2.0 Hz, 1H), 7.72 (dd, J=9.0, 1.5 Hz, 1H).
(2) Synthesis of (S)-7ring-NO₂: Pd(OAc)₂ (43 mg, 0.19 mmol, 3 mol %), SPhos (152 mg, 0.37 mmol, 6 mol %) and K₃PO₄ (2.65 g, 12.48 mmol, 2.0 equiv) were added to a dry tube equipped with a magnetic stir bar. The tube was evacuated and backfilled with nitrogen, the evacuation and back fill procedure was repeated for another twice. Then (S)-7ring-OH (2.28 g, 6.24 mmol, 1.0 equiv) and toluene (40 mL) were added under a nitrogen atmosphere. The tube was sealed and the mixture was stirred at a temperature of 100-110° C. for 2.5 days, cooled down to ambient temperature. The solvent was removed under reduced pressure and the residue was purified through column chromatography on silica gel using petroleum ether/ethyl acetate (100:1) as eluent to obtain the desired product (S)-7ring-NO₂ as yellow oily liquid 160 g in 9% yield. ¹H NMR (500 MHz, CDCl₃): δ(ppm) δ 2.32-2.45 (m, 2H), 4.33-4.37 (m, 1H), 4.41-4.46 (m, 1H), 5.10-5.12 (m, 1H), 6.63 (dd, J=9.0, 8.0 Hz, 1H), 7.11-7.13 (m, 1H), 7.33-7.37 (m, 1H), 7.40-7.42 (m, 2H), 7.44-7.46 (m, 2H), 7.87 (dd, J=8.5, 1.5 Hz, 1H), 7.90 (s, 1H).
(3) Synthesis of (S)-7ring-NH₂: (S)-7ring-NO₂ (160 mg, 0.56 mmol, 1.0 equiv), SnCl₂·2H₂O (505 mg, 2.24 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (4 mL:4 mL) were added under a nitrogen atmosphere. The flask was sealed and the mixture was stirred at a temperature of 78° C. for 16 hours, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/ethyl acetate (10:1-5:1) as eluent to obtain the desired product (S)-7ring-NH₂ as a yellow oily liquid 76 mg in 53% yield. ¹H NMR (500 MHz, CDCl₃): δ(ppm) 2.23-2.27 (m, 2H), 3.30 (s, 3H), 4.01-4.06 (m, 1H), 4.33-4.39 (m, 1H), 4.48-4.53 (m, 1H), 6.49 (dd, J=7.5, 1.5 Hz, 1H), 6.55 (dd, J=8.0, 1.5 Hz, 1H), 6.71 (t, J=8.0 Hz, 1H), 7.30-7.33 (m, 1H), 7.37-7.40 (m, 2H), 7.45-7.47 (m, 2H).
(4) Synthesis of (S)-7ring-L1: (S)-7ring-NH₂ (76 mg, 0.30 mmol, 1.0 equiv), 1-Cl (141 mg, 0.33 mmol, 1.0 equiv), Pd₂(dba)₃ (8.0 mg, 0.009 mmol, 3 mol %), JohnPhos (5 mg, 0.018 mmol, 6 mol %) and tBuONa (58 mg, 0.60 mmol, 2.0 equiv) were added to a dry tube equipped with a magnetic stir bar. The tube was evacuated and backfilled with nitrogen, the evacuation and back fill procedure was repeated for another twice. Then toluene (3 mL) was added under a nitrogen atmosphere. The tube was sealed and the mixture was stirred at a temperature of 100° C. for 12 hours, cooled down to ambient temperature. The solvent was removed under reduced pressure and the residue was purified through column chromatography on silica gel using petroleum ether/ethyl acetate (30:1) as eluent to obtain the desired product (S)-7ring-L1 as a yellow foamy solid 174 mg in 83% yield.
(5) Synthesis of (S)-7ring-LD1: (S)-7ring-L1 (174 mg, 0.25 mmol, 1.0 equiv), NH₄PF₆ (82 mg, 0.5 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (3 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (10:1) first, then dichloromethane/ethyl acetate (10:1) as eluent to obtain the desired product (S)-7ring-LD1 as a gray-white, foamy solid 60 mg in 28% yield. ¹H NMR (500 MHz, DMSO-d₆): δ(ppm) δ 1.29 (s, 9H), 1.31 (s, 9H), 2.75-2.78 (m, 2H), 4.38-4.43 (m, 1H), 4.49-4.54 (m, 1H), 6.01 (t, J=6.0 Hz, 1H), 7.15-7.17 (m, 2H), 7.23-7.26 (m, 2H), 7.34-7.36 (m, 1H), 7.38-7.45 (m, 5H), 7.46-7.49 (m, 5H), 7.54 (t, J=8.5 Hz, 1H), 7.65 (d, J=1.0 Hz, 1H), 7.73 (d, J=8.5 Hz, 1H), 8.23 (d, J=7.5 Hz, 1H), 8.30 (d, J=8.5 Hz, 1H), 8.57 (d, J=5.5 Hz, 1H), 9.78 (s, 1H).
(6) Synthesis of (S)-M-PtD1: (S)-7ring-LD1 (200 mg, 0.24 mmol, 1.0 equiv), Pt(COD)Cl₂ (94 mg, 0.252 mmol, 1.05 equiv) and NaOAc (60 mg, 0.72 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (15 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (10:1-1:1) as eluent to obtain the desired product (S)-M-PtD1 as a pale yellow solid 22 mg in 35% yield.

### Example 31

The synthetic route for (R)—P—PtD1 is as follows:

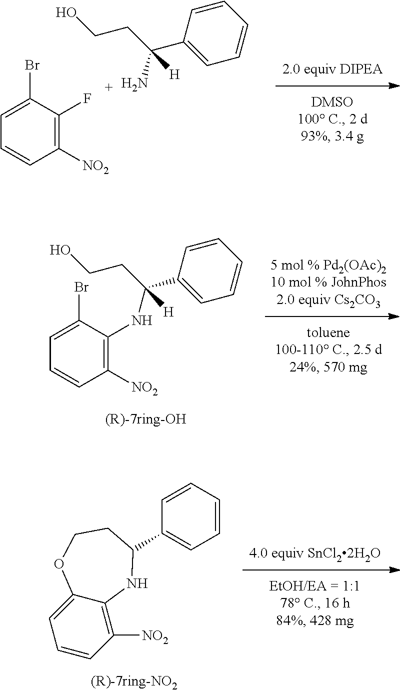

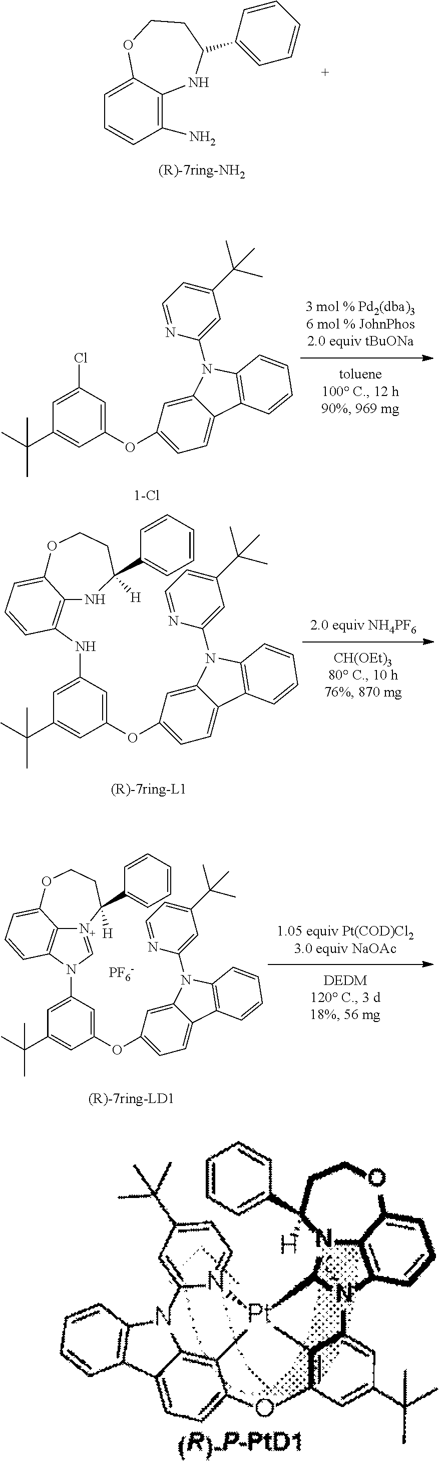

(1) Synthesis of (R)-7ring-OH: 1-bromo-2-fluoro-3-nitrobenzene (2.2 g, 10.0 mmol, 1.0 equiv), (R)-3-amino-3-phenylpropan-1-ol (1.64 g, 11.0 mmol, 1.0 equiv) were added to a dry tube equipped with a magnetic stir bar. The tube was evacuated and backfilled with nitrogen, the evacuation and back fill procedure was repeated for another twice. Then N,N-Diisopropylethylamine (DIPEA) (2.58 g, 20 mmol, 2.0 equiv) and DMSO (20 mL) were added under a nitrogen atmosphere. The tube was sealed and the mixture was stirred at a temperature of 100° C. for 2 days, cooled down to ambient temperature. The mixture was diluted with plenty of ethyl acetate (EA), filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate (10:1-5:1) as eluent to obtain the desired product (R)-7ring-OH as a yellow oily liquid 3.4 g in 93% yield.
(2) Synthesis of (R)-7ring-NO₂: Pd(OAc)₂ (92 mg, 0.42 mmol, 3 mol %), SPhos (254 mg, 0.85 mmol, 6 mol %) and K₃PO₄ (5.53 g, 16.98 mmol, 2.0 equiv) were added to a dry tube equipped with a magnetic stir bar. The tube was evacuated and backfilled with nitrogen, the evacuation and back fill procedure was repeated for another twice. Then (R)-7ring-OH (3.10 g, 8.49 mmol, 1.0 equiv) and toluene (30 mL) were added under a nitrogen atmosphere. The tube was sealed and the mixture was stirred at a temperature of 100-110° C. for 2.5 days, cooled down to ambient temperature. The solvent was removed under reduced pressure and the residue was purified through column chromatography on silica gel using petroleum ether/ethyl acetate (100:1) as eluent to obtain the desired product (R)-7ring-NO₂ as yellow oily liquid 570 g in 24% yield.
(3) Synthesis of (R)-7ring-NH₂: (R)-7ring-NO₂ (570 mg, 2.0 mmol, 1.0 equiv), SnCl₂·2H₂O (1.81 g, 8.0 mmol, 4.0 equiv) were added to a three-necked flask equipped with a magnetic stir bar. The flask was evacuated and backfilled with nitrogen, the evacuation and back fill procedure was repeated for another twice. Then ethanol and ethyl acetate (15 mL:15 mL) were added under a nitrogen atmosphere. The flask was sealed and the mixture was stirred at a temperature of 78° C. for 16 hours, cooled down to ambient temperature. The pH of the reaction mixture was adjusted to around 7-8 using a saturated NaHCO₃ solution until there was no gas to generate. Then the mixture was diluted with plenty of ethyl acetate filtered, the solid was washed with ethyl acetate. The filtrate was washed with water for three times, dried over sodium sulphate, then filtered, and the filtrate was concentrated under reduced pressure. The residue was purified through column chromatography on silica gel using petroleum ether/ethyl acetate/ethyl acetate (10:1-5:1) as eluent to obtain the desired product (R)-7ring-NH₂ as a white oily liquid 428 mg in 84% yield. ¹H NMR (500 MHz, CDCl₃): δ(ppm) 2.21-2.29 (m, 2H), 3.33 (s, 3H), 4.01-4.06 (m, 1H), 4.33-4.39 (m, 1H), 4.49-4.53 (m, 1H), 6.49 (dd, J=8.0, 1.5 Hz, 1H), 6.55 (dd, J=8.0, 1.5 Hz, 1H), 6.71 (t, J=8.0 Hz, 1H), 7.30-7.34 (m, 1H), 7.38-7.41 (m, 2H), 7.46-7.47 (m, 2H).
(4) Synthesis of (R)-7ring-L1: (R)-7ring-NH₂ (390 mg, 1.53 mmol, 1.0 equiv), 1-Cl (717 mg, 1.68 mmol, 1.0 equiv), Pd₂(dba)₃ (42 mg, 0.046 mmol, 3 mol %), JohnPhos (27 mg, 0.092 mmol, 6 mol %) and tBuONa (294 mg, 3.06 mmol, 2.0 equiv) were added to a dry tube equipped with a magnetic stir bar. The tube was evacuated and backfilled with nitrogen, the evacuation and back fill procedure was repeated for another twice. Then toluene (5 mL) was added under a nitrogen atmosphere. The tube was sealed and the mixture was stirred at a temperature of 100° C. for 12 hours, cooled down to ambient temperature. The solvent was removed under reduced pressure and the residue was purified through column chromatography on silica gel using petroleum ether/ethyl acetate (30:1) as eluent to obtain the desired product (R)-7ring-L1 as a yellow foamy solid 969 mg in 90% yield.
(5) Synthesis of (R)-7ring-LD1: (R)-7ring-L1 (939 mg, 1.34 mmol, 1.0 equiv), NH₄PF₆ (437 mg, 2.68 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (3 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (10:1) first, then dichloromethane/ethyl acetate (10:1) as eluent to obtain the desired product (R)-7ring-LD1 as a gray-white, foamy solid 870 mg in 76% yield. ¹H NMR (500 MHz, DMSO-d₆): δ(ppm) 1.29 (s, 9H), 1.31 (s, 9H), 2.75-2.78 (m, 2H), 4.38-4.43 (m, 1H), 4.50-4.54 (m, 1H), 6.01 (t, J=6.0 Hz, 1H), 7.15-7.17 (m, 2H), 7.23-7.26 (m, 2H), 7.32-7.36 (m, 1H), 7.38-7.43 (m, 5H), 7.44-7.48 (m, 5H), 7.54 (t, J=8.0 Hz, 1H), 7.65 (d, J=1.0 Hz, 1H), 7.73 (d, J=8.5 Hz, 1H), 8.23 (d, J=7.5 Hz, 1H), 8.30 (d, J=8.5 Hz, 1H), 8.57 (d, J=5.0 Hz, 1H), 9.78 (s, 1H).
(6) Synthesis of (R)—P—PtD1: (R)-7ring-LD1 (200 mg, 0.24 mmol, 1.0 equiv), Pt(COD)Cl₂ (94 mg, 0.252 mmol, 1.05 equiv) and NaOAc (60 mg, 0.72 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (15 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (10:1-1:1) as eluent to obtain the desired product (R)—P—PtD1 as a pale yellow solid 56 mg in 18% yield.

### Example 32

The synthetic route for (R,S)-M-PtE1 is as follows:

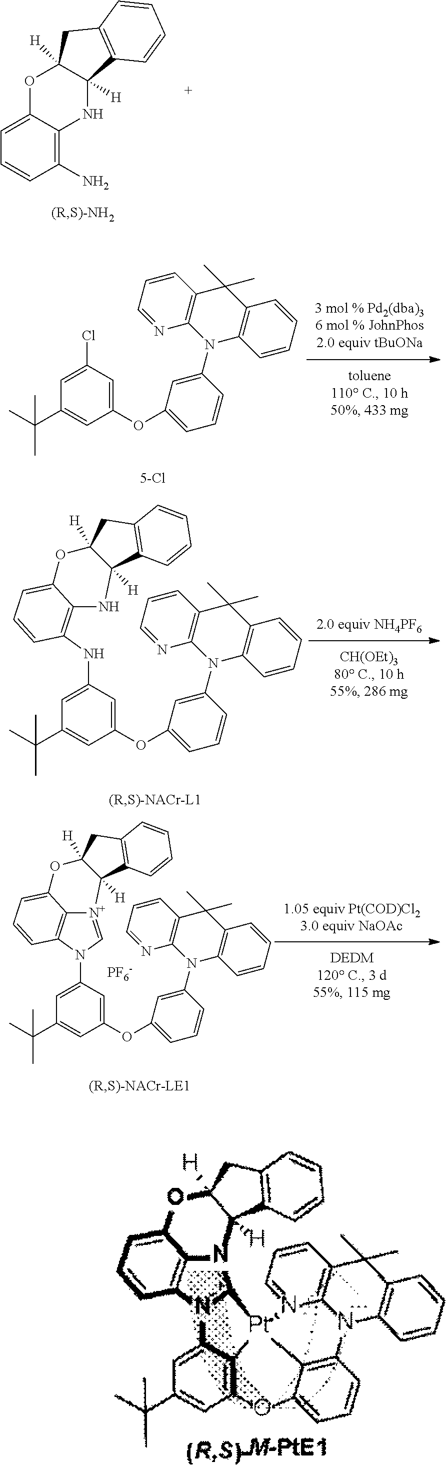

(1) Synthesis of (R,S)—NAcr-L1: (R,S)—NH₂ (309 mg, 1.30 mmol, 1.0 equiv), 5-Cl (609 mg, 1.30 mmol, 1.0 equiv), Pd₂(dba)₃ (36 mg, 0.039 mmol, 3 mol %), JohnPhos (24 mg, 0.078 mmol, 6 mol %) and tBuONa (250 mg, 2.60 mmol, 2.0 equiv) were added to a dry tube equipped with a magnetic stir bar. The tube was evacuated and backfilled with nitrogen, the evacuation and back fill procedure was repeated for another twice. Then toluene (6 mL) was added under a nitrogen atmosphere. The tube was sealed and the mixture was stirred at a temperature of 110° C. for 10 hours, cooled down to ambient temperature. The solvent was removed under reduced pressure and the residue was purified through column chromatography on silica gel using petroleum ether/ethyl acetate (30:1-15:1) as eluent to obtain the desired product (R,S)—NAcr-L1 as a yellow foamy solid 433 mg in 50% yield.
(2) Synthesis of (R,S)—NAcr-LE1: (R,S)—NAcr-L1 (425 mg, 0.63 mmol, 1.0 equiv), NH₄PF₆ (205 mg, 1.26 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (8 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (5:1) first, then dichloromethane/ethyl acetate (40:1) as eluent to obtain the desired product (R,S)—NAcr-LE1 as a brown foamy solid 286 mg in 55% yield.
(3) Synthesis of (R,S)-M-PtE1: (R,S)—NAcr-LE1 (200 mg, 0.24 mmol, 1.0 equiv), Pt(COD)Cl₂ (94 mg, 0.252 mmol, 1.05 equiv) and NaOAc (60 mg, 0.72 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (15 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (R,S)-M-PtE1 as a pale yellow solid 115 mg in 55% yield. ¹H NMR (500 MHz, DMSO-d₆): δ(ppm)¹H NMR (500 MHz, DMSO-d₆): δ (ppm) 1.33 (s, 3H), 1.46 (s, 9H), 1.67 (s, 3H), 3.17-3.23 (m, 2H), 5.21 (t, J=4.0 Hz, 1H), 5.43 (t, J=7.5 Hz, 1H), 6.17 (d, J=4.0 Hz, 1H), 6.74 (t, J=7.5 Hz, 1H), 6.81 (m, 2H), 6.92 (dd, J=7.5, 1.5 Hz, 1H), 6.96 (dd, J=7.5, 5.5 Hz, 1H), 7.02-7.09 (m, 3H), 7.11 (d, J=7.5 Hz, 1H), 7.16-7.26 (m, 2H), 7.29-7.35 (m, 2H), 7.47 (dd, J=8.0, 1.5 Hz, 1H), 7.52 (d, J=1.0 Hz, 1H), 7.70 (d, J=8.0 Hz, 1H), 7.76-7.84 (m, 1H), 9.27-9.34 (m, 1H).

### Example 33

The synthetic route for (R,S)-M-PtE1 is as follows:

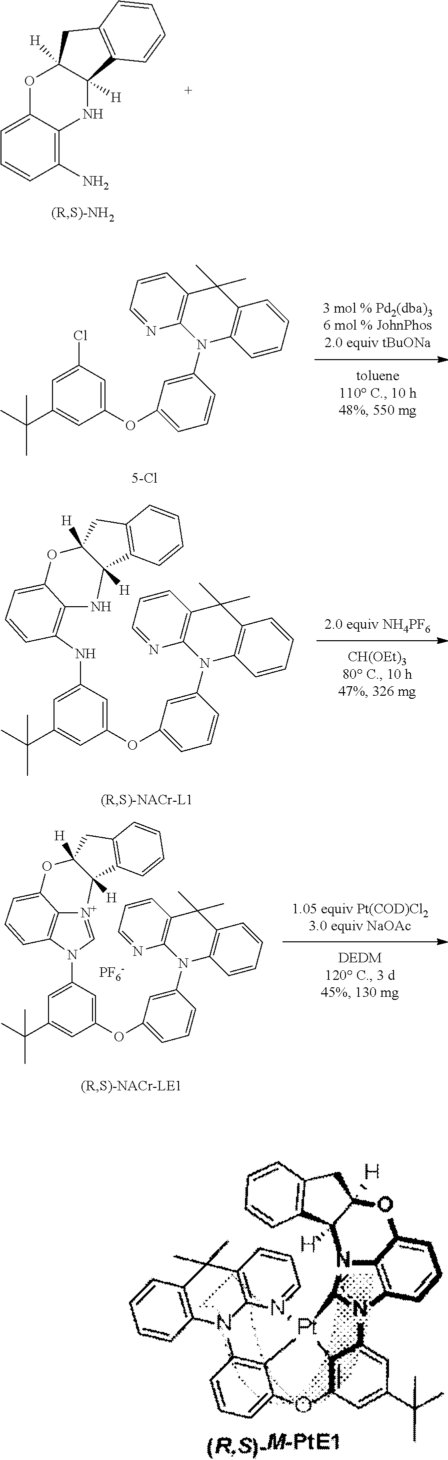

(1) Synthesis of (S,R)—NAcr-L1: (S,R)—NH₂ (405 mg, 1.70 mmol, 1.0 equiv), 5-Cl (800 mg, 1.70 mmol, 1.0 equiv), Pd₂(dba)₃ (47 mg, 0.051 mmol, 3 mol %), JohnPhos (30 mg, 0.0102 mmol, 6 mol %) and tBuONa (327 mg, 3.40 mmol, 2.0 equiv) were added to a dry tube equipped with a magnetic stir bar. The tube was evacuated and backfilled with nitrogen, the evacuation and back fill procedure was repeated for another twice. Then toluene (7 mL) was added under a nitrogen atmosphere. The tube was sealed and the mixture was stirred at a temperature of 110° C. for 10 hours, cooled down to ambient temperature. The solvent was removed under reduced pressure and the residue was purified through column chromatography on silica gel using petroleum ether/ethyl acetate (30:1-15:1) as eluent to obtain the desired product (S,R)—NAcr-L1 as a yellow foamy solid 550 mg in 48% yield.
(2) Synthesis of (S,R)—NAcr-LE1: (S,R)—NAcr-L1 (550 mg, 0.82 mmol, 1.0 equiv), NH₄PF₆ (267 mg, 1.64 mmol, 2.0 equiv) were added to a dry tube equipped with a magnetic stir bar. The tube was evacuated and backfilled with nitrogen, the evacuation and back fill procedure was repeated for another twice. Then CH(OEt)₃ (4 mL) was added under a nitrogen atmosphere. The tube was sealed and the mixture was stirred at a temperature of 80° C. for 10 hours, cooled down to ambient temperature. The solvent was removed under reduced pressure and the residue was purified through column chromatography on silica gel using petroleum ether/ethyl acetate (5:1) first, then dichloromethane/ethyl acetate (40:1) as eluent to obtain the desired product (S,R)—NAcr-LE1 as a brown foamy solid 326 mg in 47% yield. ¹H NMR (500 MHz, DMSO-d₆): δ(ppm) 1.35 (s, 9H), 1.62 (d, J=1.0 Hz, 6H), 3.32-3.37 (m, 1H), 3.59 (dd, J=16.5, 4.5 Hz, 1H), 5.47-5.49 (m, 1H), 6.25-6.28 (m, 1H), 6.40 (d, J=4.5 Hz, 1H), 6.90-6.92 (m, 1H), 6.95-6.98 (m, 2H), 7.17-7.20 (m, 3H), 7.31-7.33 (m, 1H), 7.35 (d, J=7.5 Hz, 1H), 7.39 (d, J=8.5 Hz, 1H), 7.41-7.44 (m, 2H), 7.45-7.50 (m, 3H), 7.50-7.53 (m, 1H), 7.64-7.65 (m, 2H), 7.69 (t, J=8.0 Hz, 1H), 7.84 (s, 1H), 7.85-7.86 (m, 1H), 10.49 (s, 1H).
(3) Synthesis of (S,R)—P—PtE1: (S,R)—NAcr-LE1 (280 mg, 0.34 mmol, 1.0 equiv), Pt(COD)Cl₂ (135 mg, 0.36 mmol, 1.05 equiv) and NaOAc (84 mg, 1.02 mmol, 3.0 equiv) were added to a dry tube equipped with a magnetic stir bar. The tube was evacuated and backfilled with nitrogen, the evacuation and back fill procedure was repeated for another twice. Subsequently, diethylene glycol dimethyl ether (DEDM) (20 mL) were added under a nitrogen atmosphere, and the reaction mixture was bubbled with nitrogen for 30 minutes. The tube was sealed and the mixture was stirred at a temperature of 120° C. for 3 days, cooled down to ambient temperature. The solvent was removed under reduced pressure and the residue was purified through column chromatography on silica gel using petroleum ether/dichloromethane (2:1) as eluent to obtain the desired product (S,R)—P—PtE1 as a pale yellow solid 130 mg in 45% yield. ¹H NMR (500 MHz, DMSO-d₆): δ(ppm) 1.33 (s, 3H), 1.46 (s, 9H), 1.67 (s, 3H), 3.20 (d, J=17.0 Hz, 1H), 3.26 (dd, J=16.0, 3.5 Hz, 1H), 5.20 (t, J=4.0 Hz, 1H), 5.43 (t, J=7.5 Hz, 1H), 6.16 (d, J=3.5 Hz, 1H), 6.74 (t, J=8.0 Hz, 1H), 6.80-6.82 (m, 2H), 6.92 (dd, J=8.0, 1.0 Hz, 1H), 6.95 (dd, J=7.5, 5.5 Hz, 1H), 7.03-7.08 (m, 3H), 7.11 (d, J=7.5 Hz, 1H), 7.19 (td, J=7.0, 1.0 Hz, 1H), 7.23 (td, J=8.0, 1.5 Hz, 1H), 7.30-7.33 (m, 2H), 7.47 (dd, J=7.5, 1.5 Hz, 1H), 7.52 (d, J=1.5 Hz, 1H), 7.69 (d, J=8.0 Hz, 1H), 7.78 (dd, J=7.5, 1.5 Hz, 1H), 9.31 (dd, J=5.5, 1.0 Hz, 1H).

### Example 34

The synthetic route for (R)—P—PtF1 is as follows:

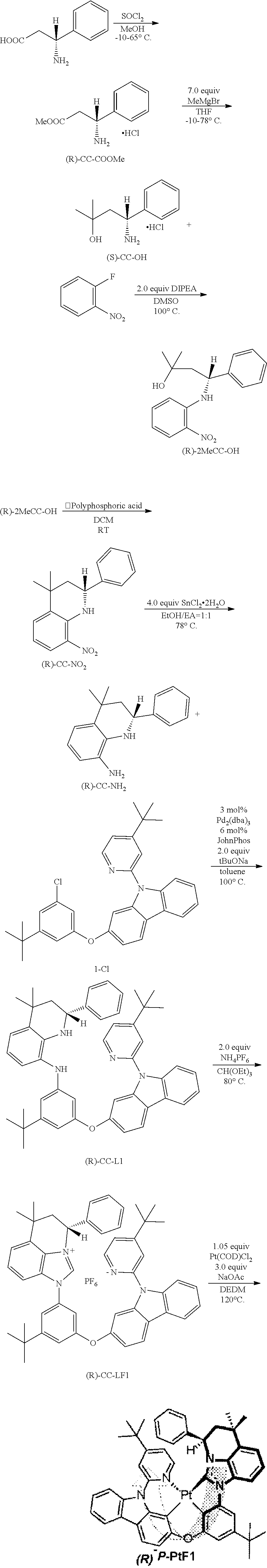

(1) Synthesis of (R)—CC—COOMe: (R)-3-amino-3-phenylpropanoic acid (5 g, 30.27 mmol, 1.0 equiv) and methanol (50 mL) were added to a dry three-necked flask equipped with a magnetic stir bar. Subsequently, the mixture was stirred at −10° C. while SOCl₂ (15 mL) was slowly added and stirred for 30 minutes. Then the mixture was heated to 65° C. for 1.5 days, cooled down to ambient temperature. The solvent was removed under reduced pressure and the residue was purified through recrystallization using methanol/petroleum ether as solvent to obtain the desired product (R)—CC—COOMe as a white solid 3.66 g in 68% yield.
(2) Synthesis of (R)—CC—OH: following the synthesis procedure of (R)-iBu-OH in example 14, the intermediate (R)—CC—COOMe was used to replace (R)-iBu-COOMe to obtain the product.
(3) Synthesis of (R)-2MeCC—OH: following the synthesis procedure of (R)-2MeiBu-OH in example 14, the intermediate (R)—CC—OH was used to replace (R)-2MeiBu-OH and substitute 1-bromo-2-fluoro-3-nitrobenzene with 1-fluoro-2-nitrobenzene to obtain the product.
(4) Synthesis of (R)—CC—NO₂: referring to the method using polyphosphoric acid to facilitate cyclization for obtaining the target product, you can consult the article “J. Org. Chem. 2020, 85, 108-117” for detailed experimental procedures and conditions.
(5) Synthesis of (R)—CC—NH₂: following the synthesis procedure of (R)-2MeiBu-NH₂ in example 14, the intermediate (R)—CC—NO₂ was used to replace (R)-2MeiiBu-NO₂ to obtain the product.
(6) Synthesis of (R)—CC-L1: following the synthesis procedure of (R)-2MeiBu-L7 in example 14, the intermediate (R)—CC—NH₂ was used to replace (R)-2MeiBu-NH₂ to obtain the product.
(7) Synthesis of (R)—CC-LF1: following the synthesis procedure of (R)-2MeiBu-A7 in example 14 to obtain the product.
(8) Synthesis of (R)—CC-LF1: following the synthesis procedure of (R)-2MeiBu-M-PtA7 in example 14 to obtain the product. MS: [M+H]⁺, 902.3.

### Example 35

The synthetic route for (R)—P—PtF2 is as follows:

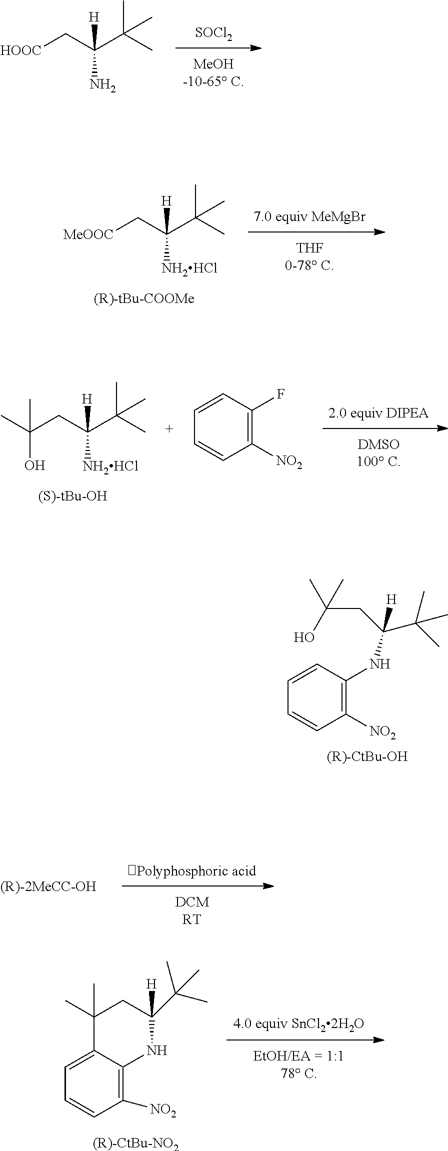

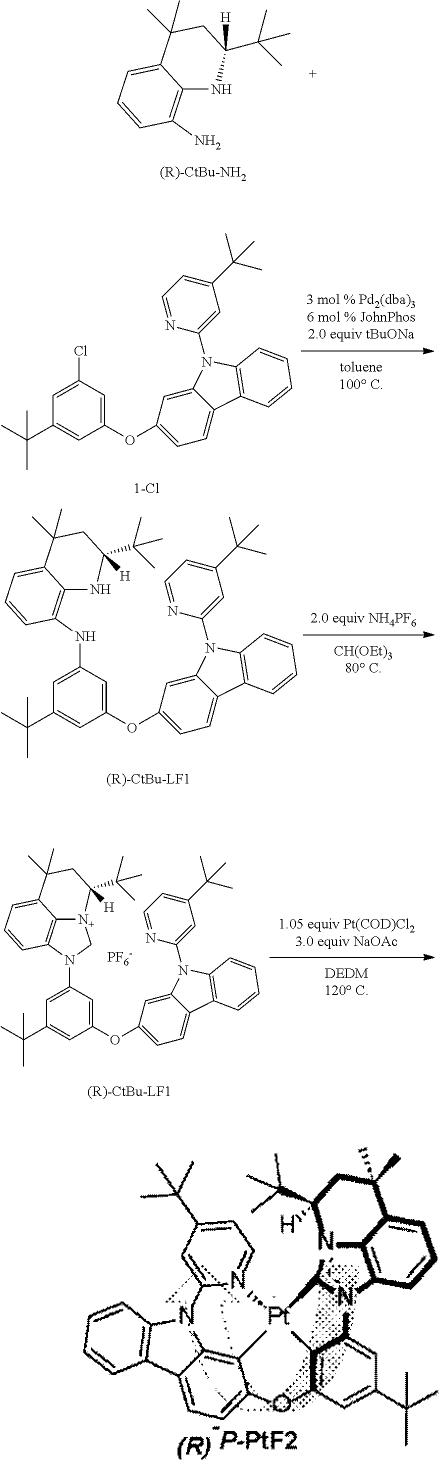

(1) Synthesis of (R)-tBu-COOMe: following the synthesis procedure of (R)-iBu-COOMe in example 14, the intermediate (R)-3-amino-4,4-dimethylpentanoic acid was used to replace D-Leucine to obtain the product.
(2) Synthesis of (R)-tBu-OH: following the synthesis procedure of (R)-iBu-OH in example 14, the intermediate (R)-tBu-COOMe was used to replace (R)-iBu-COOMe to obtain the product.
(3) Synthesis of (R)-2MetBu-OH: following the synthesis procedure of (R)-2MeiBu-OH in example 14, the intermediate (R)-2MetBu-OH was used to replace (R)-2MeiBu-OH and substitute 1-bromo-2-fluoro-3-nitrobenzene with 1-fluoro-2-nitrobenzene to obtain the product.
(4) Synthesis of (R)-CtBu-NO₂: referring to the method using polyphosphoric acid to facilitate cyclization for obtaining the target product, you can consult the article “J. Org. Chem. 2020, 85, 108-117” for detailed experimental procedures and conditions.
(5) Synthesis of (R)-CtBu-NH₂: following the synthesis procedure of (R)-2MeiBu-NH₂ in example 14, the intermediate (R)-CtBu NO₂ was used to replace (R)-2MeiBu-NO₂ to obtain the product.
(6) Synthesis of (R)-CtBu-L2: following the synthesis procedure of (R)-2MeiBu-L7 in example 14, the intermediate (R)-CtBu-NH₂ was used to replace (R)-2MeiBu-NH₂ to obtain the product.
(7) Synthesis of (R)-CtBu-LF2: following the synthesis procedure of (R)-2MeiBu-A7 in example 14 to obtain the product.
(8) Synthesis of (R)—P—PtF2: referring to the synthesis method of (R)-2MeiBu-M-PtA7 in example 14 to obtain the product. MS: [M+H]+, 882.4.

### Example 36

The synthetic route for (R)—P—PtF3 is as follows:

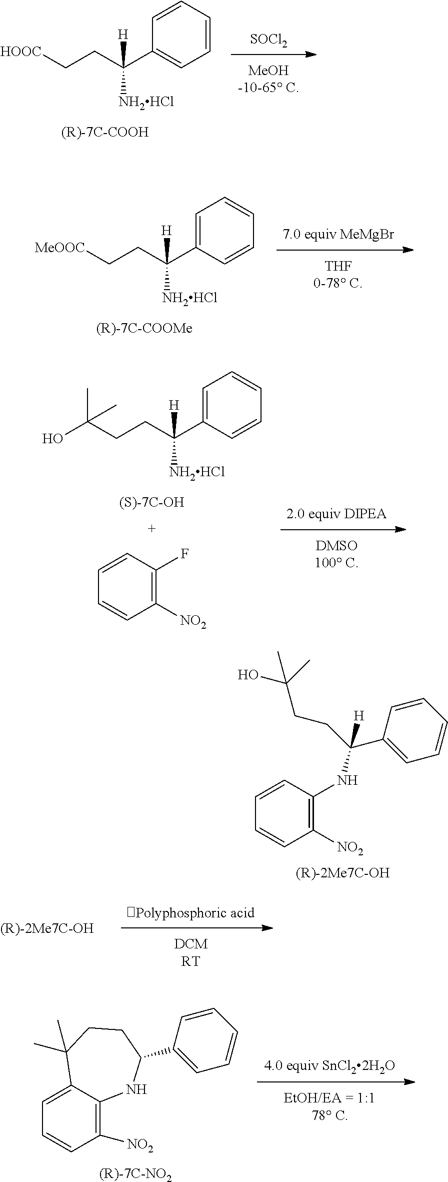

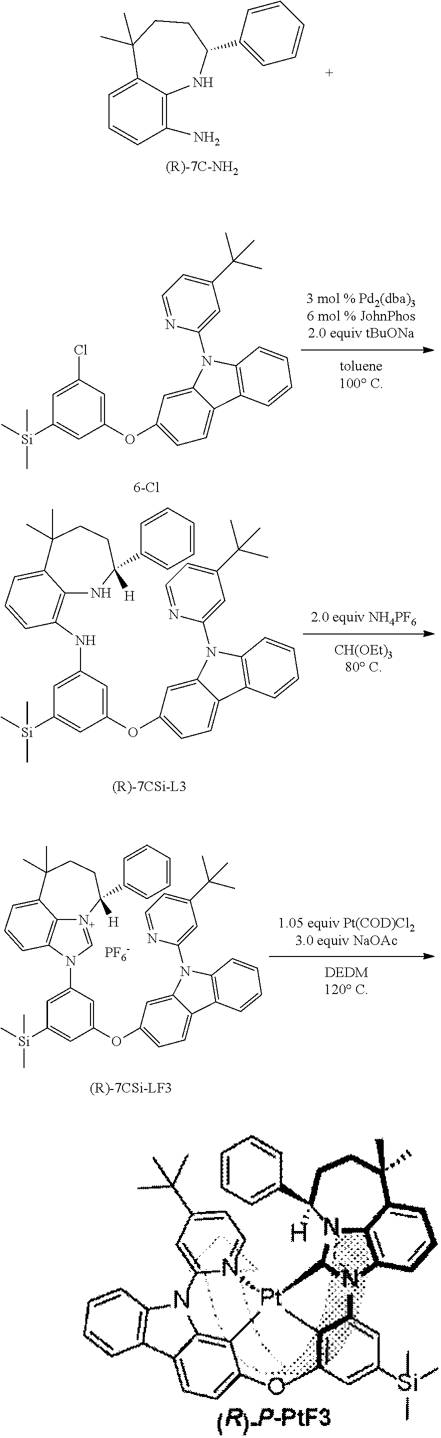

(1) Synthesis of (R)-7C—COOMe: following the synthesis procedure of (R)-iBu-COOMe in example 14, the intermediate (R)-7C—COOH was used to replace D-Leucine to obtain the product.
(2) Synthesis of (R)-7C—OH: following the synthesis procedure of (R)-iBu-OH in example 14, the intermediate (R)-7C—COOMe was used to replace (R)-iBu-COOMe to obtain the product.
(3) Synthesis of (R)-2Me7C—OH: following the synthesis procedure of (R)-2MeiBu-OH in example 14, the intermediate (R)-7C—OH was used to replace (R)-2MeiBu-OH and substitute 1-bromo-2-fluoro-3-nitrobenzene with 1-fluoro-2-nitrobenzene to obtain the product.
(4) Synthesis of (R)-7C—NO₂: referring to the method using polyphosphoric acid to facilitate cyclization for obtaining the target product, you can consult the article “J. Org. Chem. 2020, 85, 108-117” for detailed experimental procedures and conditions.
(5) Synthesis of (R)-7C—NH₂: following the synthesis procedure of (R)-2MeiBu-NH₂ in example 14, the intermediate (R)-7C NO₂ was used to replace (R)-2MeiBu-NO₂ to obtain the product.
(6) Synthesis of (R)-7CSi-L3: following the synthesis procedure of (R)-2MeiBu-L7 in example 14, the intermediate (R)-7C—NH₂ was used to replace (R)-2MeiBu-NH₂ and substitute 1-Cl with 6-Cl to obtain the product.
(7) Synthesis of (R)—P—PtF3: following the synthesis procedure of (R)-2MeiBu-A7 in example 14 to obtain the product.
(8) Synthesis of (R)—P—PtF3: referring to the synthesis method of (R)-2MeiBu-M-PtA7 in example 14 to obtain the product. MS: [M+H]+, 932.3.

### Example 37

The synthetic route for (R)-Me-M-PtA13 is as follows:

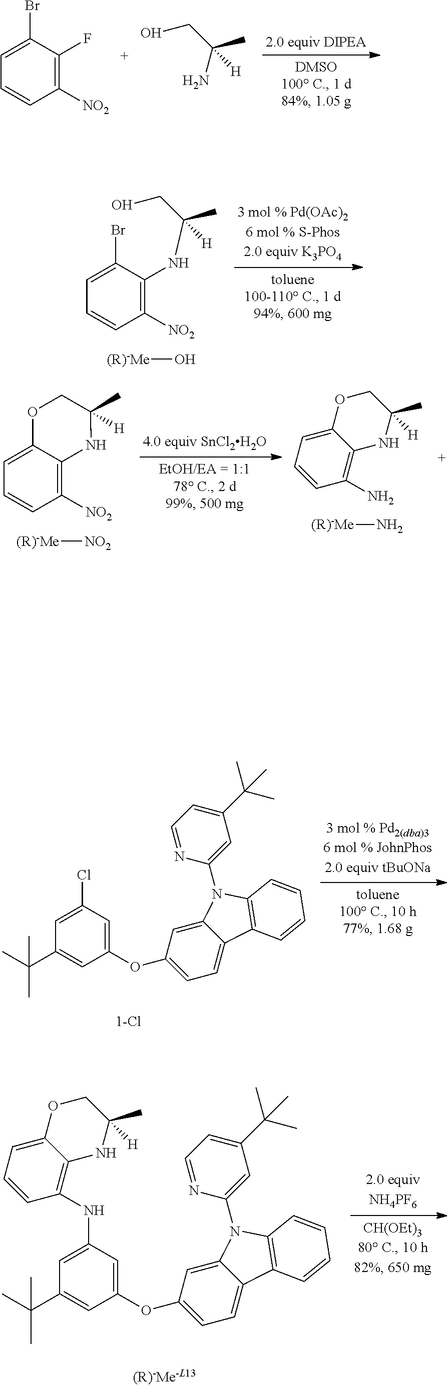

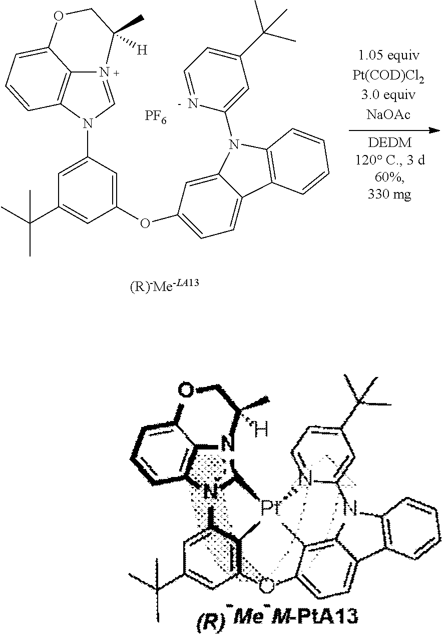

(1) Synthesis of (R)-Me-LA13: following the synthesis procedure of (R,S)-LA1 in example 1, the intermediate

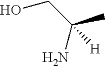

was used to replace

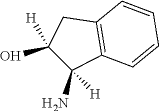 [SMILES: N[C@@H]1c2ccccc2C[C@@H]1O]

to obtain the product as a yellow foamy solid 650 mg in 83% yield. ¹H NMR (500 MHz, DMSO-d₆): δ(ppm) 1.30 (s, 9H), 1.35 (s, 9H), 1.65 (d, J=6.5 Hz, 3H), 4.25 (dd, J=12.0, 8.5 Hz, 1H), 4.76 (dd, J=11.5, 3.5 Hz, 1H), 4.85-4.92 (m, 1H), 7.20 (dd, J=8.5, 1.5 Hz, 2H), 7.25 (t, J=1.5 Hz, 1H), 7.33-7.36 (m, 2H), 7.45-7.48 (m, 3H), 7.50 (d, J=2.0 Hz, 1H), 7.53 (d, J=8.5 Hz, 1H), 7.56 (t, J=1.5 Hz, 1H), 7.68 (d, J=1.5 Hz, 1H), 7.74 (d, J=8.5 Hz, 1H), 8.24 (d, J=8.5 Hz, 1H), 8.32 (d, J=8.5 Hz, 1H), 8.58 (d, J=5.5 Hz, 1H), 12.21 (s, 1H).
(2) Synthesis of (R)-Me-M-PtA13: following the synthesis procedure of (R,S)-M-PtA1 in example 1, (R)-Me-LA13 was used to replace (R,S)-LA1 to obtain the product as a yellow solid 330 mg in 60% yield. ¹H NMR (500 MHz, DMSO-d₆): δ(ppm) 1.05 (s, 9H), 1.27 (s, 3H), 1.45 (s, 9H), 4.14 (d, J=10.0 Hz, 1H), 4.45 (d, J=10.0 Hz, 1H), 4.90 (d, J=6.5 Hz, 1H), 6.95 (d, J=8.5 Hz, 2H), 7.03 (d, J=1.5 Hz, 1H), 7.24 (s, 1H), 7.30 (d, J=8.5 Hz, 1H), 7.36-7.40 (m, 2H), 7.42-7.45 (m, 2H), 7.92 (d, J=8.0 Hz, 2H), 7.99 (d, J=8.5 Hz, 1H), 8.14 (d, J=7.0 Hz, 1H), 9.78 (d, J=6.5 Hz, 1H).

### Example 38

The synthetic route for (S)-Me-P—PtA13 is as follows:

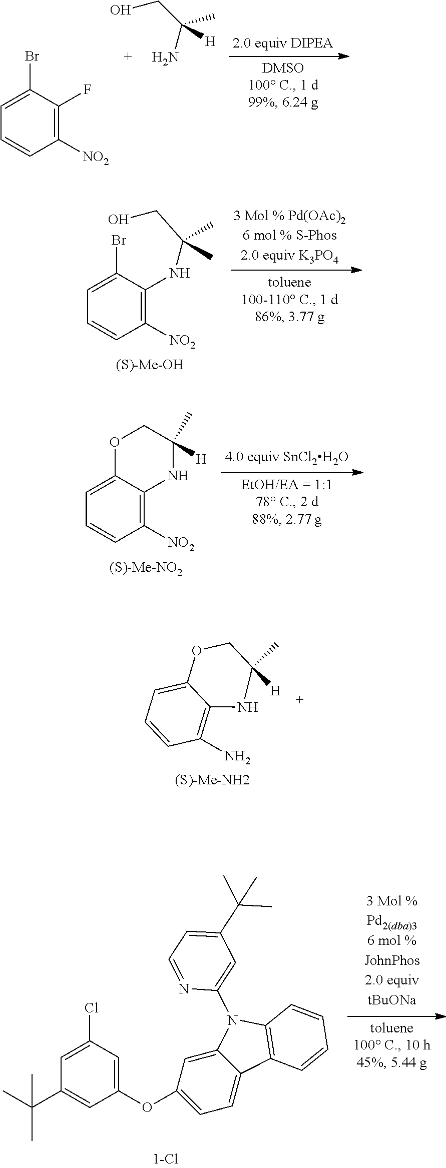

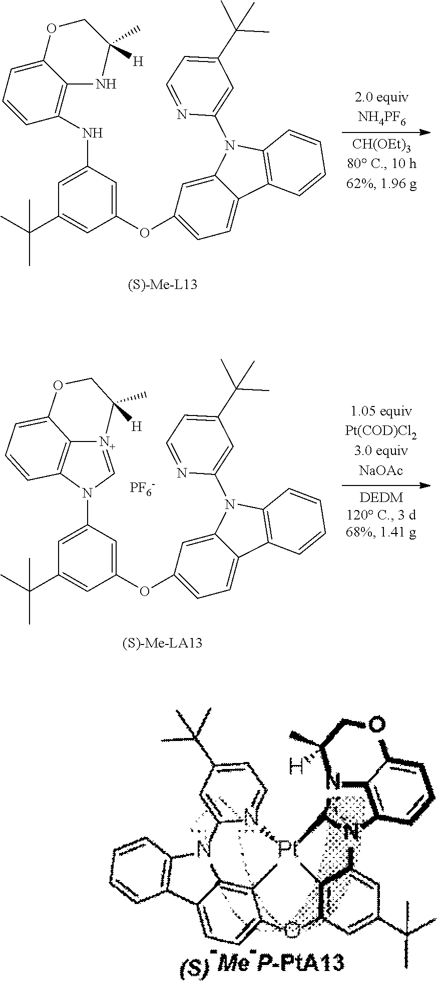

(1) Synthesis of (S)-Me-LA13: following the synthesis procedure of (S,R)-LA1 in example 2, the intermediate

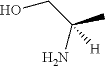

was used to replace

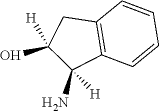 [SMILES: N[C@@H]1c2ccccc2C[C@@H]1O]

to obtain the product as a yellow foamy solid 1.96 g in 62% yield. ¹H NMR (500 MHz, DMSO-d₆): δ(ppm) 1.30 (s, 9H), 1.35 (s, 9H), 1.65 (d, J=6.5 Hz, 3H), 4.25 (dd, J=11.5, 8.0 Hz, 1H), 4.76 (dd, J=12.0, 3.5 Hz, 1H), 4.85-4.90 (m, 1H), 7.20 (dd, J=8.0, 2.0 Hz, 2H), 7.25 (t, J=1.5 Hz, 1H), 7.33-7.36 (m, 2H), 7.45-7.48 (m, 3H), 7.50 (d, J=2.0 Hz, 1H), 7.53 (d, J=8.5 Hz, 1H), 7.56 (t, J=2.5 Hz, 1H), 7.68 (d, J=1.5 Hz, 1H), 7.74 (d, J=8.0 Hz, 1H), 8.24 (d, J=7.5 Hz, 1H), 8.32 (d, J=8.5 Hz, 1H), 8.58 (d, J=5.5 Hz, 1H), 10.21 (s, 1H).
(2) Synthesis of (S)-Me-P—PtA13: following the synthesis procedure of (S,R)—P—PtA1 in example 2, (S)-Me-LA13 was used to replace (S,R)-LA1 to obtain the product as a yellow solid 1.41 g in 68% yield. ¹H NMR (500 MHz, DMSO-d₆): δ(ppm) 1.01 (s, 9H), 1.29 (s, 3H), 1.45 (s, 9H), 4.22 (d, J=10.5 Hz, 1H), 4.46 (d, J=9.5 Hz, 1H), 4.90 (d, J=7.0 Hz, 1H), 6.95 (d, J=8.0 Hz, 1H), 7.03 (d, J=1.5 Hz, 2H), 7.30 (d, J=8.5 Hz, 2H), 7.35-7.41 (m, 2H), 7.43-7.50 (m, 2H), 7.91 (d, J=8.0 Hz, 1H), 7.95 (s, 1H), 8.00 (d, J=8.5 Hz, 1H), 8.14 (d, J=7.5 Hz, 1H), 9.78 (d, J=6.0 Hz, 1H).

### Example 39

The synthetic route for (R)-iPr-M-PtA14 is as follows:

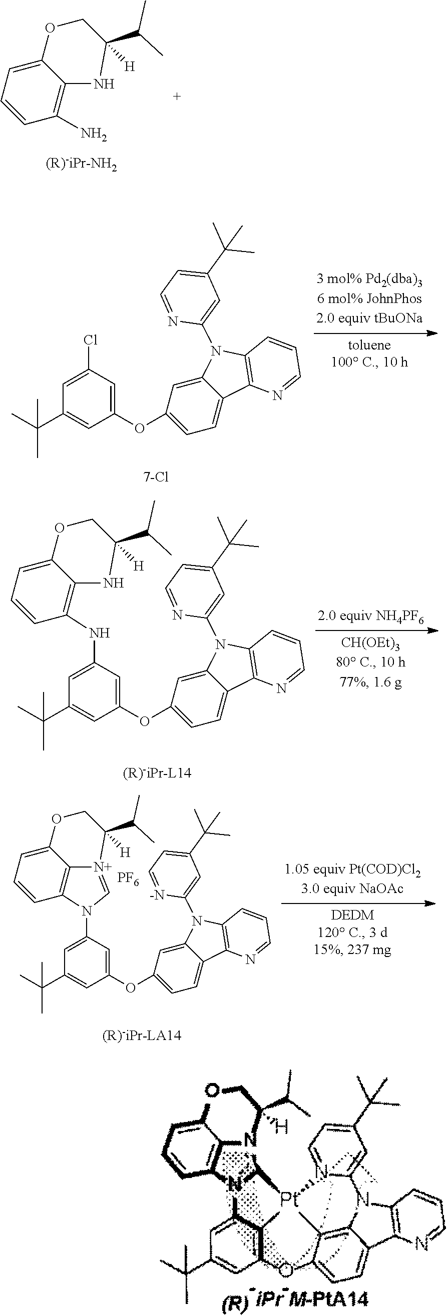

(1) Synthesis of (R)-iPr-LA14: following the synthesis procedure of (R)-iPr-LA4 in example 9, the intermediate 7-Cl was used to replace 1-Cl to obtain the product as a yellow solid 1.6 g in 77% yield. ¹H NMR (500 MHz, DMSO-d₆): δ(ppm) 1.04 (dd, J=9.0, 7.0 Hz, 6H), 1.28 (s, 9H), 1.36 (s, 9H), 2.35-2.42 (m, 1H), 3.97-4.03 (m, 1H), 4.52 (dd, J=12.0, 3.0 Hz, 1H), 4.74-4.79 (m, 1H), 4.79 (dd, J=12.0, 4.0 Hz, 1H), 7.20 (d, J=8.0 Hz, 1H), 7.28 (dd, J=8.5, 2.0 Hz, 1H), 7.34-7.36 (m, 2H), 7.47-7.50 (m, 2H), 7.52-7.57 (m, 3H), 7.62 (t, J=2.0 Hz, 1H), 7.70 (d, J=1.0 Hz, 1H), 8.16 (dd, J=8.0, 1.0 Hz, 1H), 8.37 (d, J=8.5 Hz, 1H), 8.58-8.60 (m, 2H), 10.23 (s, 1H).
(2) Synthesis of (R)-iPr-M-PtA14: following the synthesis procedure of (R)-iPr-M-PtA4 in example 9, (R)-iPr-LA14 was used to replace (R)-iPr-LA4 to obtain the product as a yellow solid 237 mg in 15% yield. ¹H NMR (500 MHz, DMSO-d₆): δ(ppm) 0.67 (dd, J=12.0, 7.0 Hz, 6H), 1.18 (s, 9H), 1.46 (s, 9H), 2.08-2.14 (m, 1H), 4.31 (d, J=10.5 Hz, 1H), 4.62 (d, J=4.0 Hz, 1H), 4.82 (d, J=11.5 Hz, 1H), 6.93 (d, J=8.0 Hz, 1H), 7.06 (d, J=3.5 Hz, 1H), 7.10 (d, J=5.5 Hz, 1H), 7.36-7.41 (m, 3H), 7.47 (dd, J=8.0, 4.0 Hz, 1H), 7.58 (d, J=8.5 Hz, 1H), 7.89 (d, J=2.0 Hz, 1H), 7.96 (d, J=8.0 Hz, 1H), 8.42 (dd, J=8.0, 1.0 Hz, 1H), 8.58 (dd, J=4.5, 1.0 Hz, 1H), 9.78 (d, J=6.5 Hz, 1H).

### Example 40

The synthetic route for (S)-iPr—P—PtA14 is as follows:

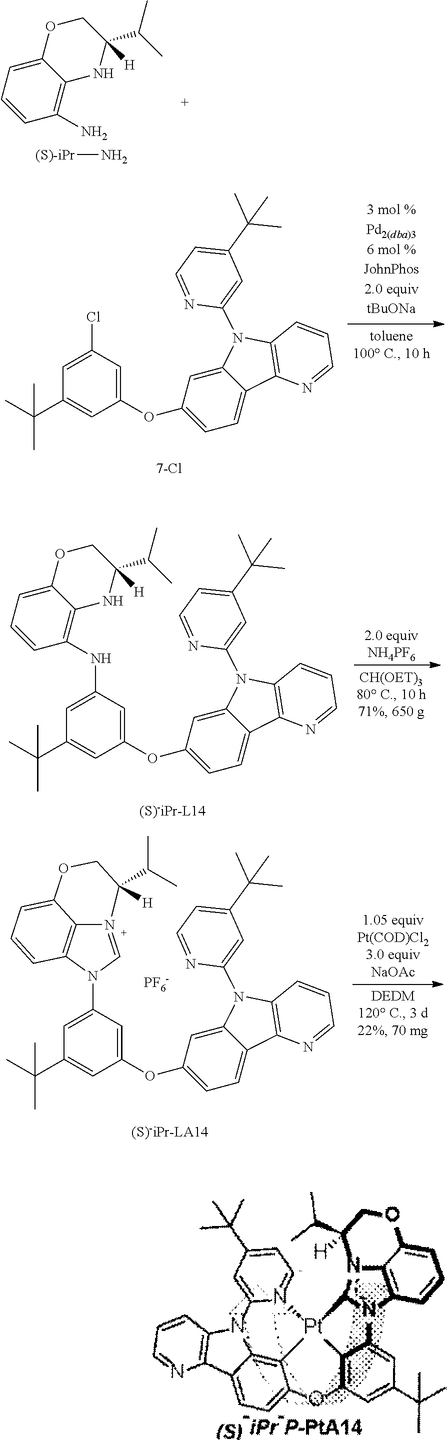

(1) Synthesis of (S)-iPr-LA14: following the synthesis procedure of (S)-iPr-LA4 in example 10, the intermediate 7-Cl was used to replace 1-Cl to obtain the product as a yellow solid 650 mg in 71% yield. ¹H NMR (500 MHz, DMSO-d₆): δ(ppm) 1.04 (dd, J=9.0, 7.0 Hz, 6H), 1.28 (s, 9H), 1.36 (s, 9H), 2.35-2.42 (m, 1H), 3.98-4.01 (m, 1H), 4.52 (dd, J=12.0, 3.0 Hz, 1H), 4.73-4.76 (m, 1H), 4.79 (dd, J=12.5, 4.0 Hz, 1H), 7.20 (d, J=8.0 Hz, 1H), 7.28 (dd, J=8.5, 2.0 Hz, 1H), 7.34-7.35 (m, 2H), 7.47-7.50 (m, 2H), 7.52-7.56 (m, 3H), 7.62 (t, J=1.5 Hz, 1H), 7.69 (d, J=1.0 Hz, 1H), 8.15 (dd, J=8.8, 1.0 Hz, 1H), 8.37 (d, J=8.5 Hz, 1H), 8.57-8.60 (m, 2H), 10.23 (s, 1H).
(2) Synthesis of (S)-iPr—P—PtA14: following the synthesis procedure of (S)-iPr—P—PtA4 in example 10, (S)-iPr-LA14 was used to replace (S)-iPr-LA14 to obtain the product as a yellow solid 70 mg in 22% yield. ¹H NMR (500 MHz, DMSO-d₆): δ(ppm) 0.70 (dd, J=12.5, 7.0 Hz, 6H), 1.26 (s, 9H), 1.45 (s, 9H), 2.10-2.17 (m, 1H), 4.46 (d, J=11.0 Hz, 1H), 4.62 (d, J=1.5 Hz, 1H), 4.85 (d, J=12.0 Hz, 1H), 6.93 (d, J=8.0 Hz, 1H), 7.04 (d, J=1.5 Hz, 1H), 7.21 (d, J=4.0 Hz, 1H), 7.36 (d, J=8.0 Hz, 1H), 7.39 (t, J=8.0, 1H), 7.46 (d, J=2.0 Hz, 1H), 7.49 (dd, J=8.5, 1.5 Hz, 1H), 7.65 (d, J=8.0 Hz, 1H), 7.96 (d, J=8.0 Hz, 2H), 8.44 (dd, J=8.5, 1.0 Hz, 1H), 8.58 (dd, J=5.0, 1.5 Hz, 1H), 9.74 (d, J=6.0 Hz, 1H).

### Example 41

The synthetic route for (R)-tBu-M-PtA15 is as follows:

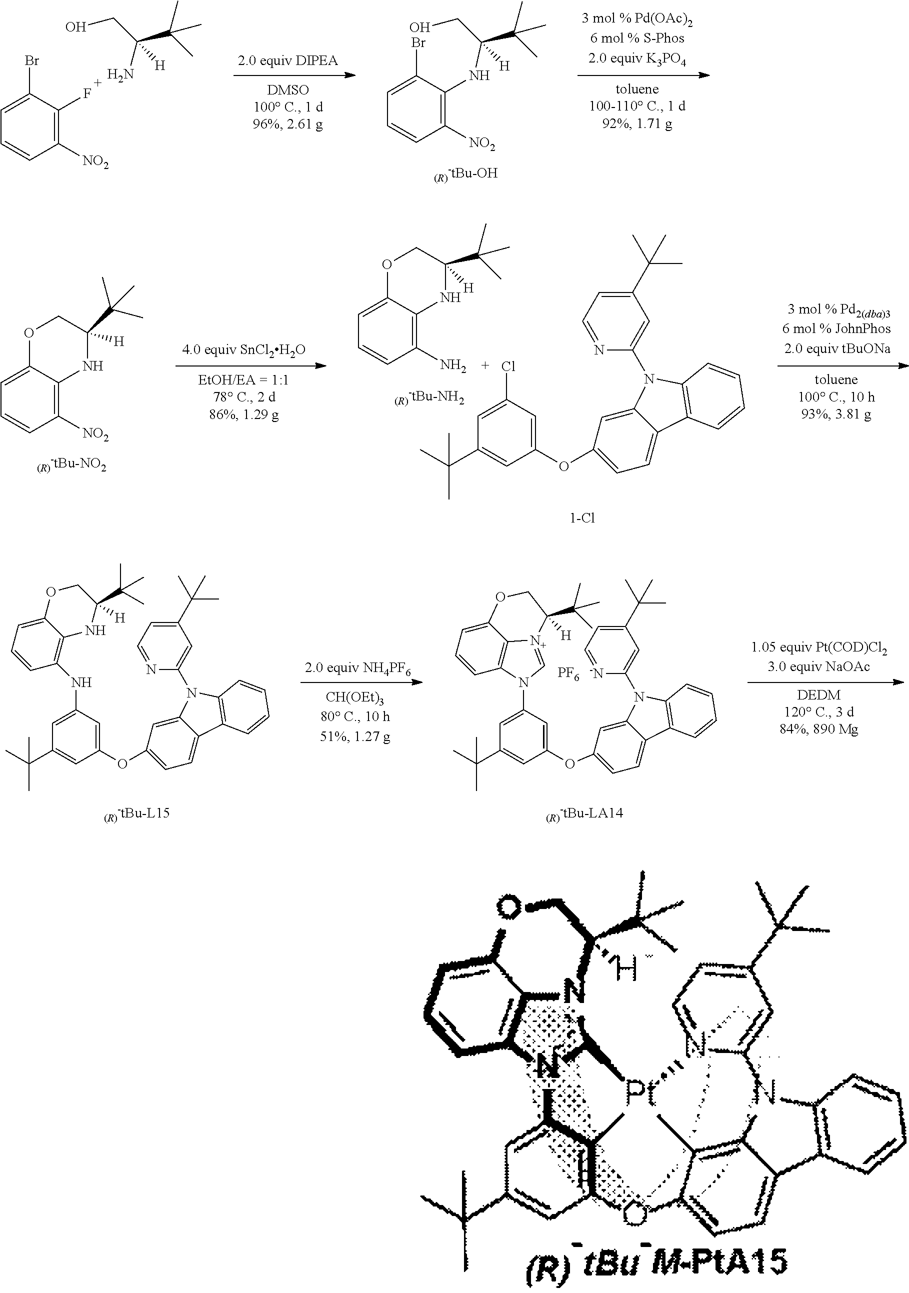

(1) Synthesis of (R)-tBu-LA15: following the synthesis procedure of (R,S)-LA1 in example 1, the intermediate

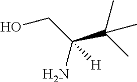

was used to replace

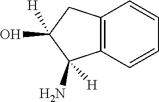 [SMILES: N[C@@H]1c2ccccc2C[C@@H]1O]

to obtain the product (R)-tBu-LA15 as a yellow foamy solid 1.27 g in 51% yield.
(2) Synthesis of (R)-tBu-M-PtA15: following the synthesis procedure of (R,S)-M-PtA1 in example 1, (R)-tBu-LA15 was used to replace (R,S)-LA1 to obtain the product as a yellow solid 890 mg in 84% yield. ¹H NMR (500 MHz, DMSO-d₆): δ(ppm) 0.80 (s, 9H), 1.28 (s, 9H), 1.45 (s, 1OH), 4.55 (d, J=12.0 Hz, 1H), 4.57 (s, 1H), 5.01 (d, J=12.0 Hz, 1H), 6.94 (d, J=8.0 Hz, 1H), 7.01 (d, J=1.5 Hz, 1H), 7.20 (dd, J=6.0, 2.0 Hz, 1H), 7.28 (d, J=8.0 Hz, 1H), 7.37-7.42 (m, 2H), 7.45-7.50 (m, 1H), 7.51 (d, J=1.5 Hz, 1H), 7.71 (d, J=8.5 Hz, 1H), 7.89 (d, J=8.5 Hz, 1H), 8.03 (d, J=8.5 Hz, 1H), 8.09 (d, J=2.0 Hz, 1H), 8.15 (d, J=7.5 Hz, 1H), 9.62 (d, J=6.0 Hz, 1H).

### Example 42

The synthetic route for (S)-tBu-P—PtA15 is as follows:

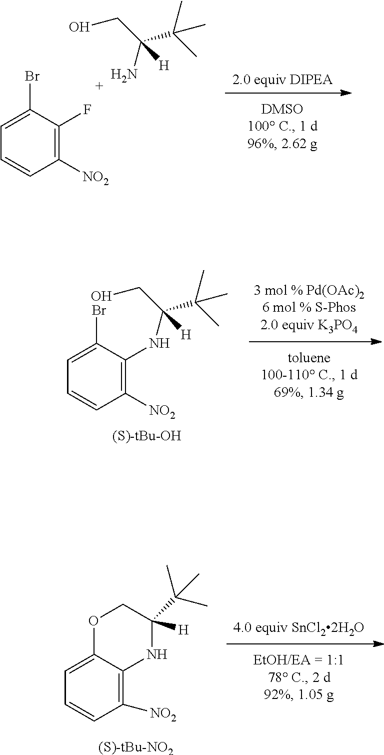

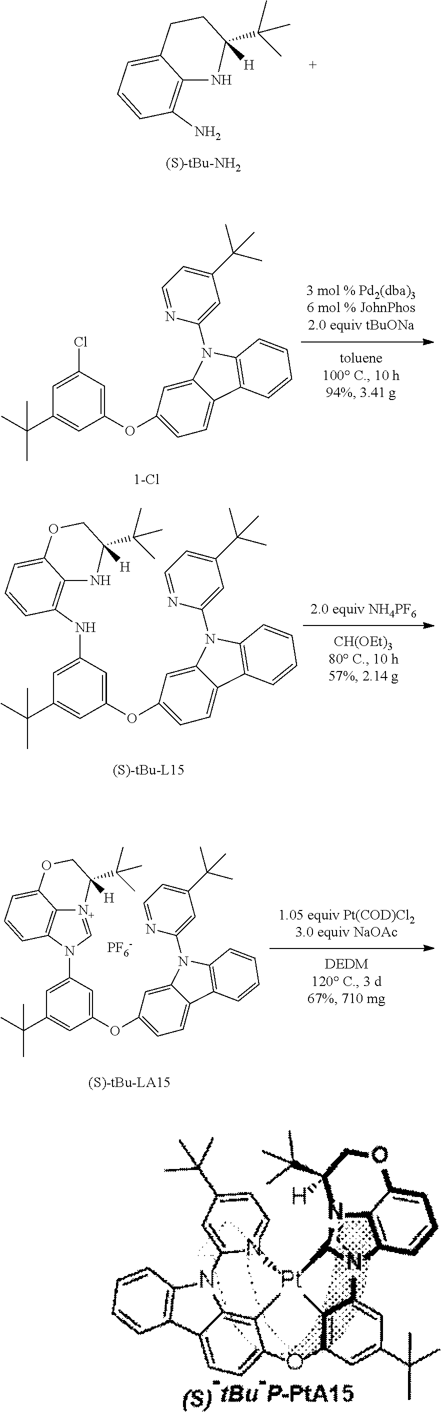

(1) Synthesis of (S)-tBu-LA15: following the synthesis procedure of (S,R)-LA1 in example 2, the intermediate

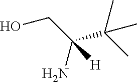

was used to replace

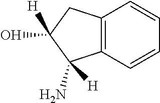 [SMILES: N[C@H]1c2ccccc2C[C@H]1O]

obtain the product as a yellow foamy solid 2.14 g in 57% yield.
(2) Synthesis of (R)-tBu-M-PtA15: following the synthesis procedure of (S,R)—P—PtA1 in example 2, (S)-tBu-LA15 was used to replace (S,R)-LA1 to obtain the product as a yellow solid 710 mg in 67% yield. ¹H NMR (500 MHz, DMSO-d₆): δ(ppm) 0.81 (s, 9H), 1.30 (s, 9H), 1.46 (s, 9H), 4.58 (d, J=9.0 Hz, 2H), 5.02 (d, J=11.5 Hz, 1H), 6.95 (d, J=8.0 Hz, 1H), 7.01 (d, J=1.5 Hz, 1H), 7.21 (dd, J=6.0, 2.0 Hz, 1H), 7.28 (d, J=8.5 Hz, 1H), 7.40 (dd, J=16.5, 8.5 Hz, 2H), 7.45-7.50 (m, 1H), 7.53 (d, J=1.5 Hz, 1H), 7.73 (d, J=8.5 Hz, 1H), 7.89 (d, J=8.0 Hz, 1H), 8.05 (d, J=8.5 Hz, 1H), 8.11 (d, J=2.0 Hz, 1H), 8.15 (d, J=7.5 Hz, 1H), 9.63 (d, J=6.0 Hz, 1H).

### Example 43

The synthetic route for (R,R)-cyh-M-PtA16 is as follows:

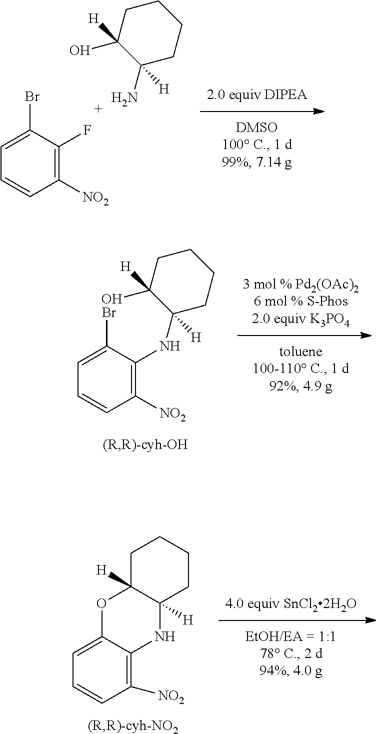

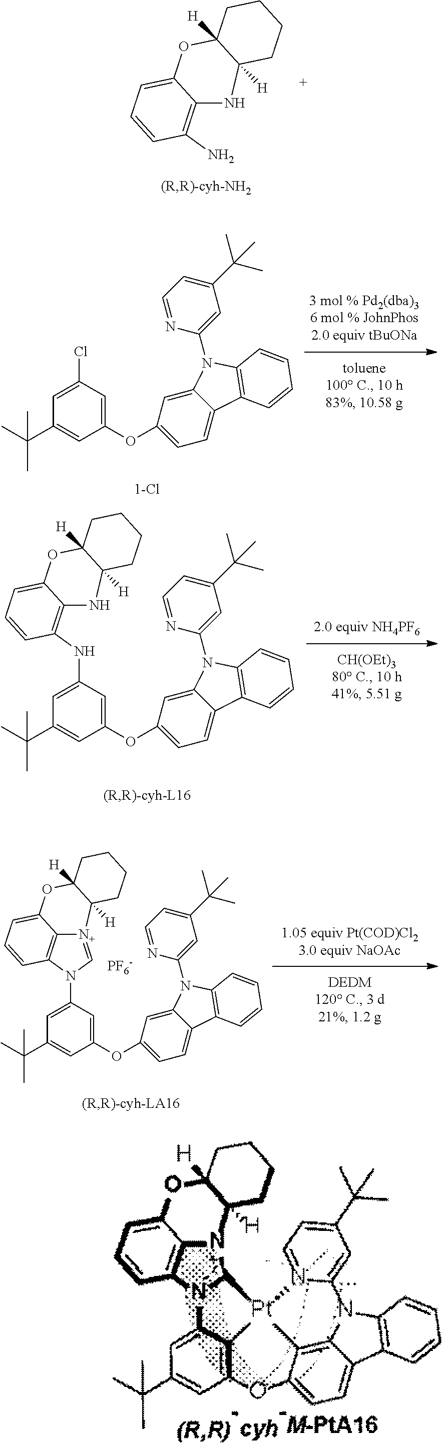

(1) Synthesis of (R,R)-cyh-LA16: following the synthesis procedure of (R,S)-LA1 in example 1, the intermediate

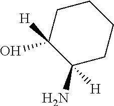

was used to replace

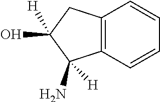 [SMILES: N[C@@H]1c2ccccc2C[C@@H]1O]

to obtain the product (R,R)-cyh-LA16 as a yellow foamy solid 5.51 g in 42% yield. ¹H NMR (500 MHz, DMSO-d₆): δ(ppm) 1.29 (s, 9H), 1.35 (s, 9H), 1.45-1.59 (m, 2H), 1.70-1.77 (m, 1H), 1.79-1.89 (m, 1H), 1.88 (d, J=12.5 Hz, 2H), 2.31-2.34 (m, 1H), 2.75-2.77 (m, 1H), 4.19-4.24 (m, 1H), 4.38-4.43 (m, 1H), 7.19-7.21 (m, 2H), 7.25 (t, J=2.5 Hz, 1H), 7.33-7.36 (m, 2H), 7.45-7.49 (m, 3H), 7.50 (d, J=2.5 Hz, 1H), 7.53-7.60 (m, 2H), 7.68 (d, J=1.0 Hz, 1H), 7.74 (d, J=8.0 Hz, 1H), 8.24 (d, J=7.5 Hz, 1H), 8.32 (d, J=8.5 Hz, 1H), 8.598 (d, J=5.5 Hz, 1H), 10.23 (s, 1H).
(2) Synthesis of (R,R)-cyh-M-PtA16: following the synthesis procedure of (R,S)-M-PtA1 in example 1, (R,R)-cyh-LA16 was used to replace (R,S)-LA1 to obtain the product as a yellow solid 1.2 g in 21% yield. ¹H NMR (500 MHz, CDCl₃): δ(ppm) 1.36 (s, 9H), 1.39-1.45 (m, 2H), 1.47 (s, 9H), 1.54-1.60 (m, 2H), 1.77-1.86 (m, 2H), 2.36-2.40 (m, 1H), 3.24 (d, J=12.5 Hz, 1H), 3.85-3.90 (m, 1H), 4.27-4.31 (m, 1H), 6.83 (d, J=8.0 Hz, 1H), 6.99 (dd, J=6.5, 2.0 Hz, 1H), 7.17 (d, J=1.5 Hz, 1H), 7.30 (t, J=8.0 Hz, 1H), 7.34-7.41 (m, 2H), 7.43 (d, J=8.5 Hz, 1H), 7.56 (d, J=1.5 Hz, 1H), 7.61 (d, J=8.0 Hz, 1H), 7.86 (d, J=8.0 Hz, 1H), 7.88 (d, J=7.5 Hz, 1H), 8.05 (dd, J=8.0, 1.5 Hz, 1H), 8.20 (d, J=2.0 Hz, 1H), 9.33 (d, J=6.5 Hz, 1H)_{∘}

### Example 44

The synthetic route for (S,S)-cyh-P—PtA16 is as follows:

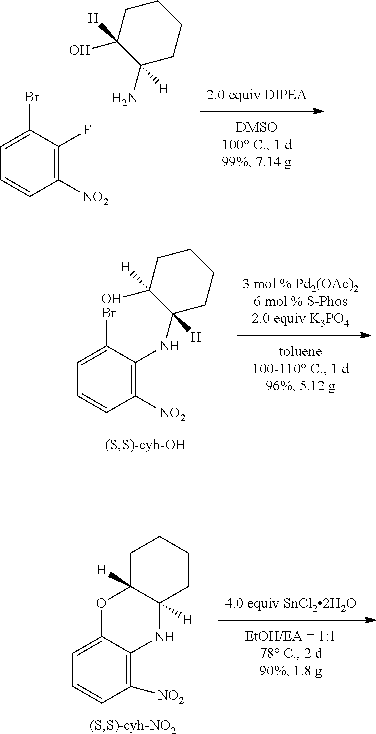

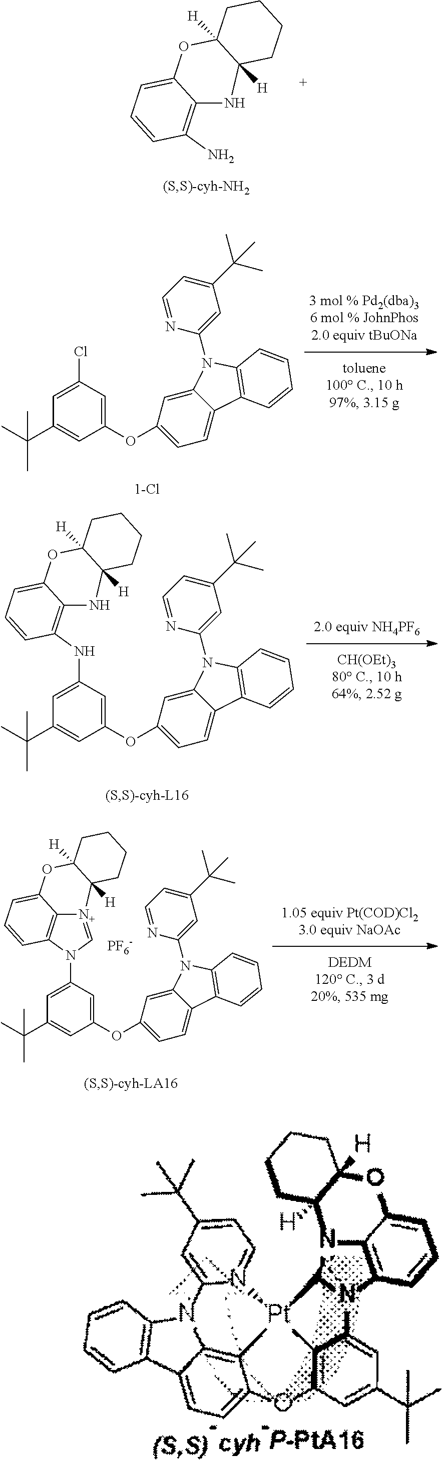

(1) Synthesis of (S)-tBu-LA15: following the synthesis procedure of (S,R)-LA1 in example 2, the intermediate

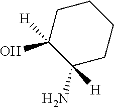

was used to replace

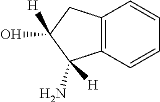 [SMILES: N[C@H]1c2ccccc2C[C@H]1O]

to obtain the product as a yellow foamy solid 2.52 g in 64% yield. ¹H NMR (500 MHz, DMSO-d₆): δ(ppm) 1.30 (s, 9H), 1.36 (s, 9H), 1.46-1.59 (m, 2H), 1.70-1.78 (m, 1H), 1.79-1.84 (m, 1H), 1.89 (d, J=12.0 Hz, 2H), 2.31-2.34 (m, 1H), 2.75-2.77 (m, 1H), 4.19-4.24 (m, 1H), 4.38-4.43 (m, 1H), 7.198-7.20 (m, 2H), 7.25 (t, J=2.5 Hz, 1H), 7.33-7.36 (m, 2H), 7.45-7.49 (m, 3H), 7.50 (d, J=2.5 Hz, 1H), 7.53-7.56 (m, 2H), 7.68 (d, J=1.0 Hz, 1H), 7.74 (d, J=8.5 Hz, 1H), 8.24 (d, J=7.5 Hz, 1H), 8.32 (d, J=8.5 Hz, 1H), 8.598 (d, J=5.5 Hz, 1H), 10.23 (s, 1H).
(2) Synthesis of (S,S)-cyh-P—PtA16: following the synthesis procedure of (S,R)—P—PtA1 in example 2, (S,S)-cyh-LA16 was used to replace (S,R)-LA1 to obtain the product as a yellow solid 535 mg in 20% yield. ¹H NMR (500 MHz, CDCl₃): δ(ppm) 0.81-0.89 (m, 1H), 0.91-0.99 (m, 1H), 1.21-1.25 (m, 1H), 1.37 (s, 9H), 1.44 (s, 9H), 1.44-1.49 (m, 1H), 1.74-1.86 (m, 2H), 2.26-2.33 (m, 1H), 2.93 (d, J=12.0 Hz, 1H), 4.07-4.12 (m, 1H), 4.61-4.66 (m, 1H), 6.96-6.98 (m, 2H), 7.27 (d, J=8.0 Hz, 1H), 7.37-7.43 (m, 3H), 7.49-7.52 (m, 1H), 7.56 (d, J=1.5 Hz, 1H), 7.80 (d, J=8.5 Hz, 1H), 7.92 (d, J=8.5 Hz, 1H), 8.06 (d, J=8.5 Hz, 1H), 8.15 (d, J=2.5 Hz, 1H), 8.17 (d, J=7.5 Hz, 1H), 9.51 (d, J=6.5 Hz, 1H).

### Example 45

The synthetic route for (S)—CS—P—PtA17 is as follows:

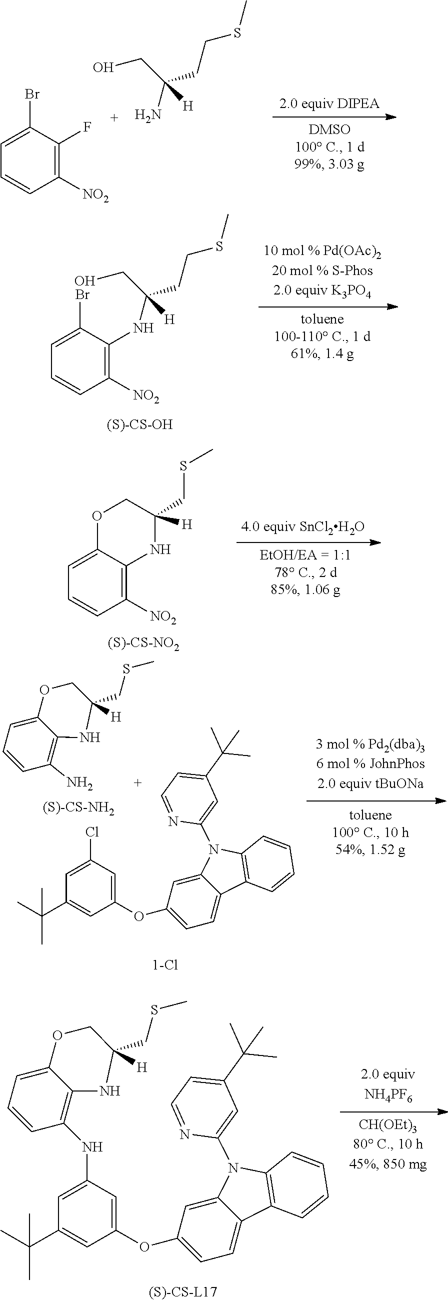

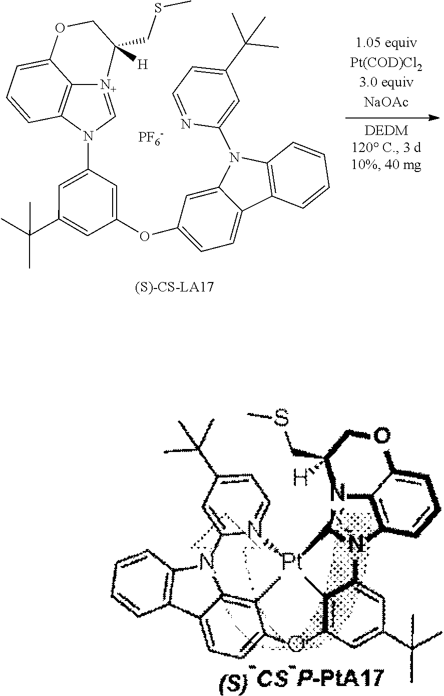

(1) Synthesis of (S)—CS-LA17: following the synthesis procedure of (S,R)-LA1 in example 2, the intermediate

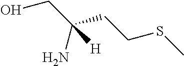

was used to replace

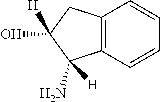 [SMILES: N[C@H]1c2ccccc2C[C@H]1O]

to obtain the product as a yellow foamy solid 850 mg in 45% yield. ¹H NMR (500 MHz, DMSO-d₆): δ(ppm) δ(ppm) 1.29 (s, 9H), 1.35 (s, 9H), 2.07 (s, 3H), 2.12-2.23 (m, 1H), 2.29-2.36 (m, 1H), 2.70 (t, J=7.5 Hz, 1H), 4.59 (dd, J=12.0, 5.0 Hz, 1H), 4.66 (dd, J=12.0, 3.0 Hz, 1H), 4.96-5.00 (m, 1H), 7.19-7.24 (m, 2H), 7.24 (t, J=2.0 Hz, 1H), 7.34-7.36 (m, 2H), 7.45-7.48 (m, 3H), 7.50 (d, J=2.0 Hz, 1H), 7.53-7.56 (m, 2H), 7.66 (d, J=1.0 Hz, 1H), 7.73 (d, J=8.0 Hz, 1H), 8.24 (d, J=7.5 Hz, 1H), 8.32 (d, J=8.5 Hz, 1H), 8.57 (d, J=6.0 Hz, 1H), 10.19 (s, 1H).
(2) Synthesis of (S,S)-cyh-P—PtA16: following the synthesis procedure of (S,R)—P—PtA1 in example 2, (S)—CS-LA17 was used to replace (S,R)-LA1 to obtain the product as a yellow solid 40 mg in 10% yield. ¹H NMR (500 MHz, CDCl₃): δ(ppm) 1.13 (s, 9H), 1.45 (s, 9H), 1.64 (s, 3H), 1.78-1.84 (m, 1H), 1.92-1.98 (m, 1H), 2.41-2.47 (m, 1H), 2.59-2.64 (m, 1H), 4.31 (d, J=10.0 Hz, 1H), 4.73 (d, J=10.5 Hz, 1H), 5.05 (t, J=7.5 Hz, 1H), 6.95 (d, J=8.0 Hz, 1H), 7.02-7.03 (m, 2H), 7.30 (d, J=8.5 Hz, 1H), 7.33 (s, 1H), 7.35-7.41 (m, 2H), 7.45-7.48 (m, 1H), 7.53 (d, J=8.5 Hz, 1H), 7.88-7.91 (m, 2H), 7.95 (d, J=1.5 Hz, 1H), 8.14 (d, J=7.5 Hz, 1H), 9.71 (d, J=6.5 Hz, 1H).

### Example 46

The synthetic route for (R)-iPr-M-PtA18 is as follows:

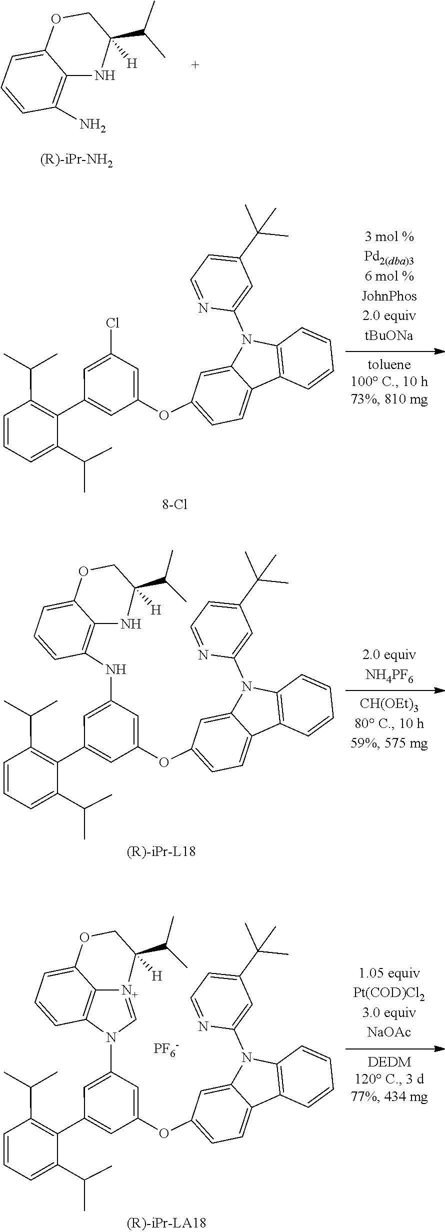

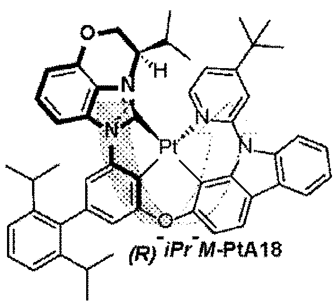

(1) Synthesis of (R)-iPr-LA18: following the synthesis procedure of (R)-iPr-LA4 in example 9, 8-Cl was used to replace 1-Cl to obtain the product as a yellow solid 575 mg in 59% yield.
(2) Synthesis of (R)-iPr-M-PtA14: following the synthesis procedure of (R)-iPr-M-PtA4 in example 9, (R)-iPr-LA1 was used to replace (R)-iP-LA4 to obtain the product as a yellow solid 434 mg in 77% yield. ¹H NMR (500 MHz, CDCl₃): δ(ppm) 0.85 (d, J=7.0 Hz, 3H), 0.88 (d, J=6.5 Hz, 3H), 1.10-1.17 (m, 12H), 1.38 (s, 9H), 2.32-2.36 (m, 1H), 2.92-2.96 (m, 1H), 2.99-3.04 (m, 1H), 4.47-4.53 (m, 1H), 4.67-4.71 (m, 1H), 4.81 (d, J=12.0 Hz, 1H), 6.80 (d, J=8.0 Hz, 1H), 6.96 (d, J=1.0 Hz, 1H), 7.00 (dd, J=6.5, 2.0 Hz, 1H), 7.21 (t, J=8.0 Hz, 1H), 7.28 (s, 2H), 7.38-7.42 (m, 3H), 7.42-7.47 (m, 3H), 7.84 (d, J=8.0 Hz, 1H), 7.92 (d, J=8.0 Hz, 1H), 8.07 (d, J=7.0 Hz, 1H), 8.26 (d, J=2.0 Hz, 1H), 9.52 (d, J=6.0 Hz, 1H).

### Example 47

The synthetic route for (S)-iPr—P—PtA18 is as follows:

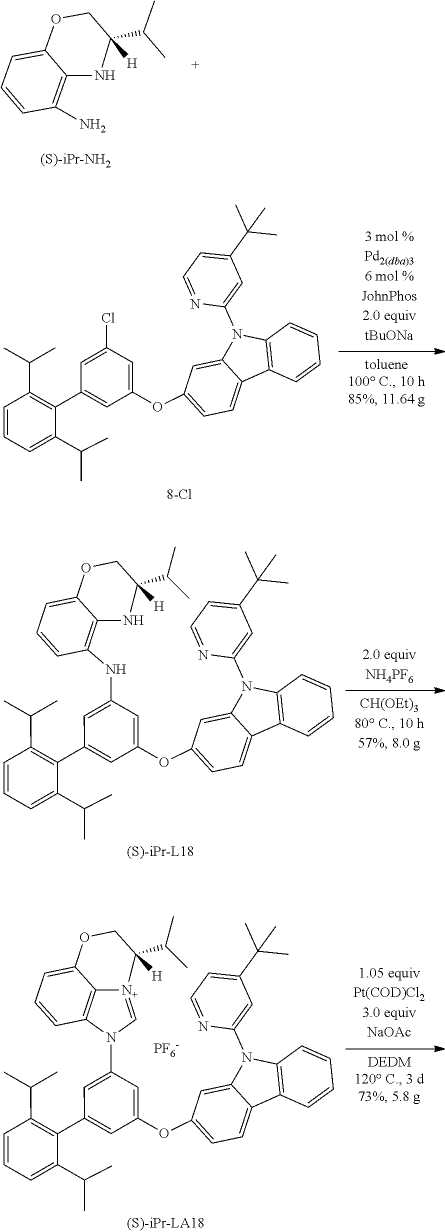

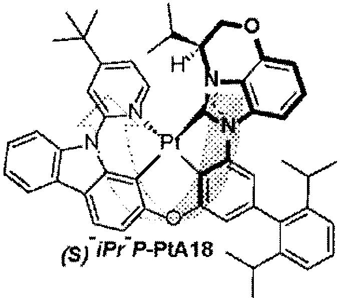

(1) Synthesis of (S)-iPr-LA18: following the synthesis procedure of (S)-iPr-LA4 in example 10, 8-Cl was used to replace 1-Cl to obtain the product as a yellow solid 8.0 g in 57% yield.
(2) Synthesis of (S)-iPr—P—PtA18: following the synthesis procedure of (S)-iPr—P—PtA4 in example 10, (S)-iPr-LA18 was used to replace (S)-iPr-LA4 to obtain the product as a yellow solid 5.8 g in 73% yield. ¹H NMR (500 MHz, DMSO-d₆): δ(ppm) 0.76 (dd, J=9.0, 7.0 Hz, 6H), 1.10-1.13 (m, 12H), 1.38 (s, 9H), 2.15-2.19 (m, 1H), 2.83-2.86 (m, 2H), 4.61 (dd, J=12.5, 2.5 Hz, 1H), 4.75 (d, J=6.0 Hz, 1H), 4.90 (d, J=12.0 Hz, 1H), 6.73 (d, J=1.5 Hz, 1H), 6.90 (d, J=8.0 Hz, 1H), 7.26-7.31 (m, 5H), 7.35 (dd, J=6.0, 2.0 Hz, 1H), 7.38-7.42 (m, 2H), 7.50-7.53 (m, 1H), 7.62 (d, J=8.5 Hz, 1H), 7.90 (d, J=8.0 Hz, 1H), 8.08 (d, J=8.5 Hz, 1H), 8.17 (d, J=8.0 Hz, 1H), 8.19 (d, J=2.0 Hz, 1H), 9.69 (d, J=6.0 Hz, 1H).

### Example 48

The synthetic route for (R)-tBu-M-PtA19 is as follows:

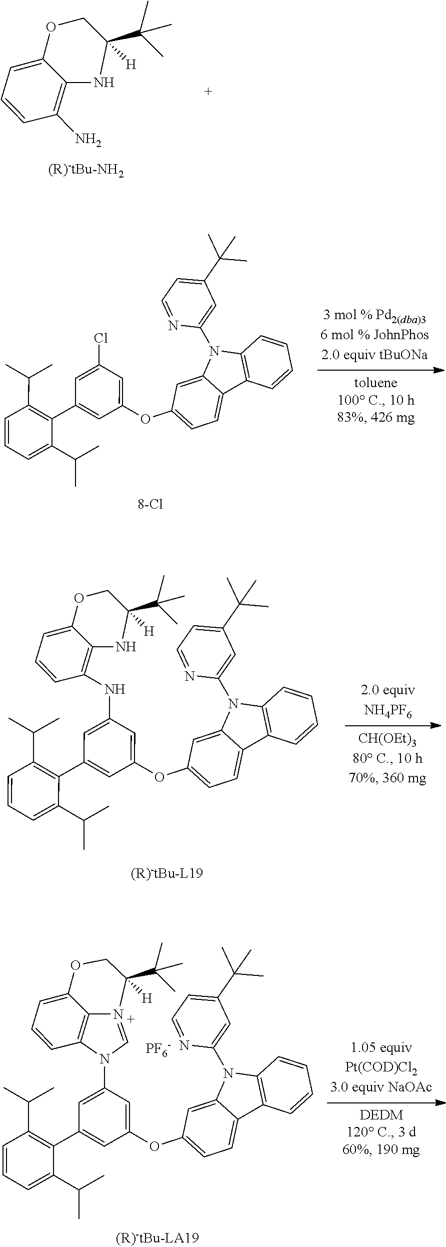

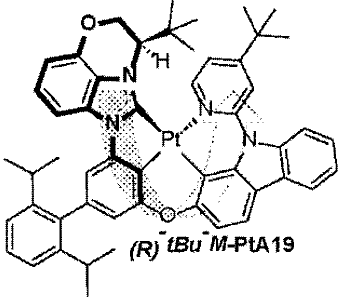

(1) Synthesis of (R)-tBu-LA19: following the synthesis procedure of (R)-tBu-LA15 in example 41, 8-Cl was used to replace 1-Cl to obtain the product as a yellow solid 360 mg in 70% yield. ¹H NMR (500 MHz, DMSO-d₆): δ(ppm) 0.91 (dd, J=9.5, 7.0 Hz, 6H), 1.07-1.12 (m, 15H), 1.31 (s, 9H), 2.55-2.59 (m, 2H), 4.33 (dd, J=13.0, 3.5 Hz, 1H), 4.71 (d, J=3.7 Hz, 1H), 5.10 (d, J=13.0 Hz, 1H), 7.04-7.05 (m, 1H), 7.20-7.24 (m, 4H), 7.33-7.37 (m, 3H), 7.45-7.50 (m, 4H), 7.60 (t, J=8.5 Hz, 1H), 7.69-7.70 (m, 2H), 7.73 (d, J=8.0 Hz, 1H), 8.24 (d, J=7.5 Hz, 1H), 8.34 (d, J=8.5 Hz, 1H), 8.56 (d, J=5.0 Hz, 1H), 10.27 (s, 1H).
(2) Synthesis of (R)-tBu-M-PtA19: following the synthesis procedure of (R)-tBu-M-PtA15 in example 41, (R)-tBu-LA19 was used to replace (R)-tBu-LA19 to obtain the product as a yellow solid 190 mg in 60% yield. ¹H NMR (500 MHz, DMSO-d₆): δ(ppm) 0.83 (s, 9H), 1.11-1.14 (m, 12H), 1.33 (s, 9H), 2.84-2.91 (m, 2H), 4.60 (s, 1H), 4.67 (dd, J=12.0, 2.5 Hz, 1H), 5.01 (d, J=11.5 Hz, 1H), 6.74 (d, J=1.5 Hz, 1H), 6.88 (d, J=8.0 Hz, 1H), 7.22-7.30 (m, 5H), 7.36-7.41 (m, 3H), 7.48-7.51 (m, 1H), 7.67 (d, J=8.5 Hz, 1H), 7.90 (d, J=8.5 Hz, 1H), 8.07 (d, J=8.0 Hz, 1H), 8.15-8.17 (m, 2H), 9.53 (d, J=7.5 Hz, 1H).

### Example 49

The synthetic route for (S)-tBu-P—PtA19 is as follows:

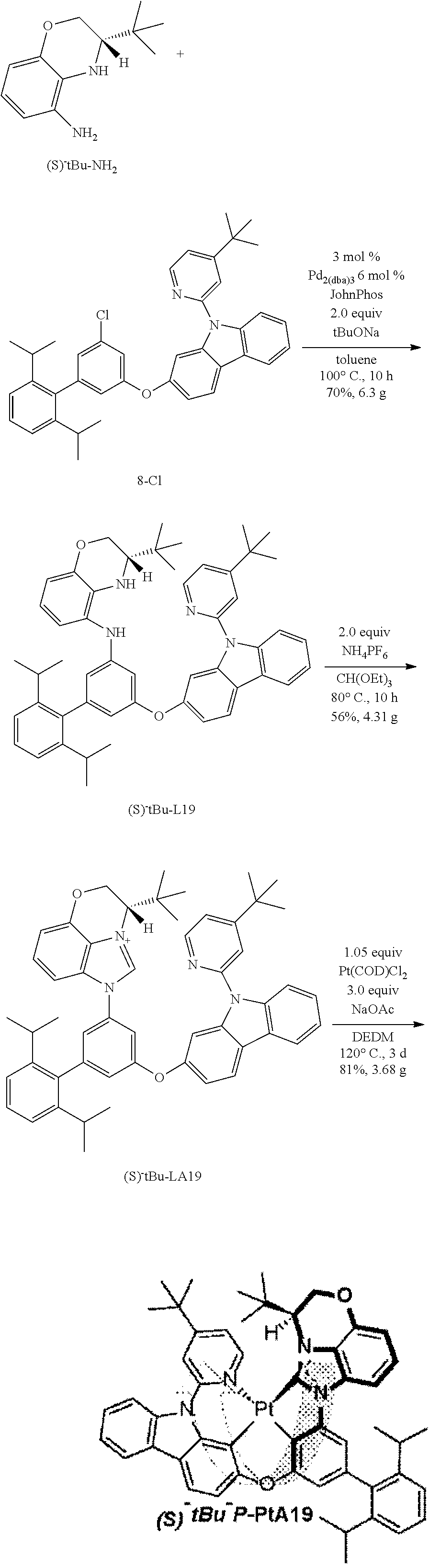 [SMILES: CC(C)(C)[C@H]1COc2cccc(N)c2N1 ; CC(C)c1cccc(C(C)C)c1-c1cc(Cl)cc(Oc2ccc3c4ccccc4n(-c4cc(C(C)(C)C)ccn4)c3c2)c1 ; CC(C)c1cccc(C(C)C)c1-c1cc(Nc2cccc3c2N[C@@H](C(C)(C)C)CO3)cc(Oc2ccc3c4ccccc4n(-c4cc(C(C)(C)C)ccn4)c3c2)c1 ; CC(C)c1cccc(C(C)C)c1-c1cc(Oc2ccc3c4ccccc4n(-c4cc(C(C)(C)C)ccn4)c3c2)cc(-n2c[n+]3c4c(cccc42)OC[C@@H]3C(C)(C)C)c1]

(1) Synthesis of (S)-tBu-LA19: following the synthesis procedure of (S)-tBu-LA15 in example 42, 8-C was used to replace 1-Cl to obtain the product as a yellow solid 4.31 g in 56% yield. ¹H NMR (500 MHz, DMSO-d₆): δ(ppm) 0.91 (d, J=7.0 Hz, 3H), 0.92 (d, J=7.0 Hz, 3H), 1.07-1.09 (m, 12H), 1.11 (d, J=6.5 Hz, 3H), 1.32 (s, 9H), 2.54-2.62 (m, 2H), 4.33 (dd, J=12.5, 3.5 Hz, 1H), 4.71 (d, J=3.0 Hz, 1H), 5.10 (d, J=13.0 Hz, 1H), 7.04 (s, 1H), 7.20-7.24 (m, 4H), 7.33-7.37 (m, 3H), 7.44-7.48 (m, 2H), 7.50 (dd, J=5.5, 2.0 Hz, 2H), 7.60 (t, J=8.5 Hz, 1H), 7.69 (d, J=2.0 Hz, 1H), 7.70 (s, 1H), 7.73 (d, J=8.5 Hz, 1H), 8.24 (d, J=8.0 Hz, 1H), 8.33 (d, J=8.5 Hz, 1H), 8.56 (d, J=5.0 Hz, 1H), 10.27 (s, 1H).
(2) Synthesis of (S)-tBu-P—PtA19: following the synthesis procedure of (S)-tBu-P—PtA15 in example 42, (S)-tBu-LA19 was used to replace (S)-tBu-LA15 to obtain the product as a yellow solid 3.68 g in 81% yield. ¹H NMR (500 MHz, DMSO-d₆): δ(ppm) 0.84 (s, 9H), 1.12-1.25 (m, 12H), 1.35 (s, 9H), 2.83-2.92 (m, 2H), 4.62 (s, 1H), 4.67 (dd, J=12.5, 3.0 Hz, 1H), 5.03 (d, J=11.5 Hz, 1H), 6.75 (d, J=1.0 Hz, 1H), 6.90 (d, J=8.0 Hz, 1H), 7.24-7.31 (m, 5H), 7.36-7.42 (m, 3H), 7.49-7.52 (m, 1H), 7.68 (d, J=8.0 Hz, 1H), 7.92 (d, J=8.0 Hz, 1H), 8.09 (d, J=8.5 Hz, 1H), 8.17-8.19 (m, 2H), 9.57 (d, J=6.0 Hz, 1H).

### Example 50

The synthetic route for (R)-iPr-M-PtA20 is as follows:

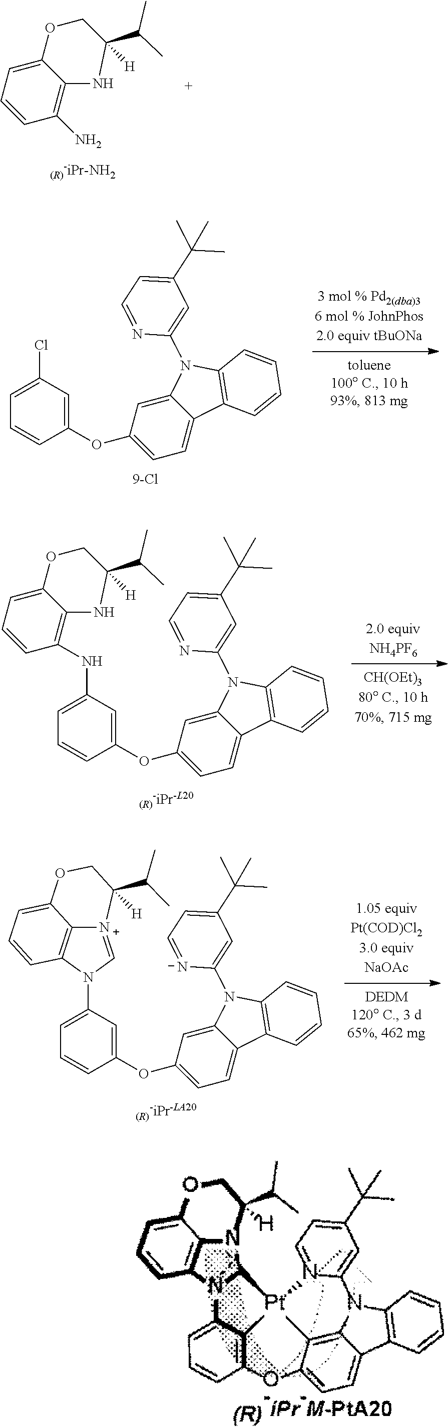

(1) Synthesis of (R)-iPr-LA20: following the synthesis procedure of (R)-iPr-LA4 in example 9, 9-Cl was used to replace 1-Cl to obtain the product as a yellow solid 715 mg in 70% yield. ¹H NMR (500 MHz, DMSO-d₆): δ(ppm) 1.03 (d, J=6.5 Hz, 3H), 1.05 (d, J=7.0 Hz, 3H), 1.31 (s, 9H), 2.37-2.43 (m, 1H), 4.54 (dd, J=12.0, 3.0 Hz, 1H), 4.73-4.83 (m, 2H), 7.19-7.23 (m, 2H), 7.33-7.37 (m, 1H), 7.40-7.44 (m, 2H), 7.45-7.49 (m, 2H), 7.50 (d, J=2.5 Hz, 1H), 7.53 (t, J=2.0 Hz, 1H), 7.56 (t, J=8.0 Hz, 1H), 7.61 (dd, J=8.0, 2.0 Hz, 1H), 7.69 (d, J=2.0 Hz, 1H), 7.73-7.79 (m, 2H), 8.25 (dd, J=7.5, 1.0 Hz, 1H), 8.34 (d, J=8.5 Hz, 1H), 8.59 (d, J=5.5 Hz, 1H), 10.25 (s, 1H).
(2) Synthesis of (R)-iPr-M-PtA20: following the synthesis procedure of (R)-iPr-M-PtA4 in example 9, (R)-iPr-LA20 was used to replace (R)-iPr-LA4 to obtain the product as a yellow solid 462 mg in 65% yield. ¹H NMR (500 MHz, DMSO-d₆): δ(ppm) 0.66 (d, J=6.5 Hz, 6H), 0.83 (s, 9H), 2.13 (q, J=6.5 Hz, 1H), 3.93 (dd, J=12.0, 3.0 Hz, 1H), 4.37 (dd, J=6.5, 3.0 Hz, 1H), 4.56 (d, J=12.0 Hz, 1H), 6.29 (d, J=6.5 Hz, 1H), 6.71 (s, 1H), 6.83 (dd, J=5.5, 2.0 Hz, 1H), 7.03-7.09 (m, 2H), 7.28-7.35 (m, 4H), 7.44 (d, J=8.5 Hz, 1H), 7.70-7.74 (m, 1H), 7.77 (d, J=2.0 Hz, 1H), 7.83 (d, J=8.5 Hz, 1H), 7.98-8.04 (m, 1H), 9.85 (d, J=6.0 Hz, 1H).

### Example 51

The synthetic route for (S)-iPr—P—PtA20 is as follows:

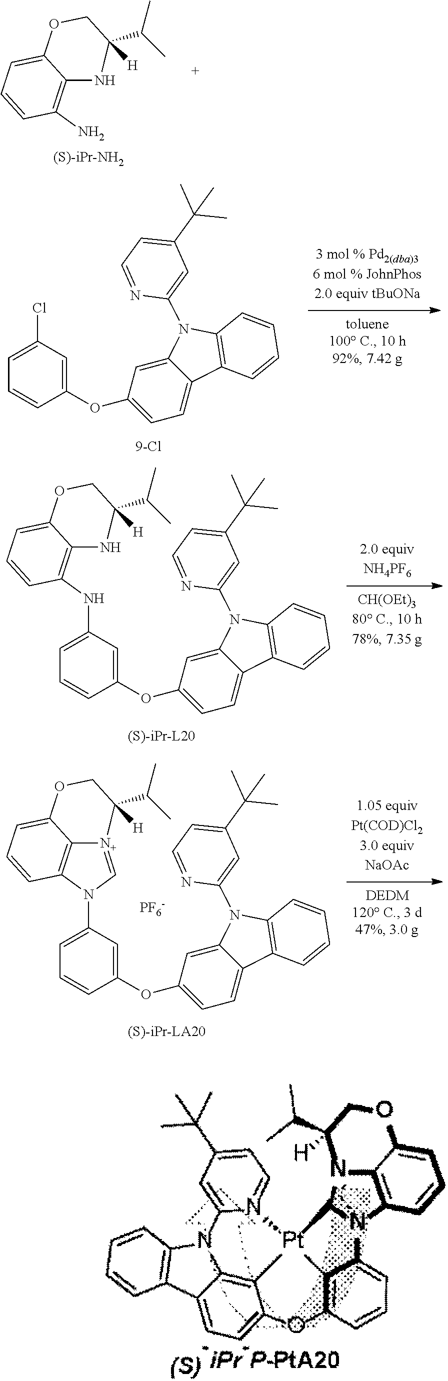

(1) Synthesis of (S)-iPr-LA20: following the synthesis procedure of (S)-iPr-LA4 in example 10, 9-Cl was used to replace 1-Cl to obtain the product as a yellow solid 7.35 g in 78% yield.
(2) Synthesis of (S)-iPr—P—PtA20: following the synthesis procedure of (S)-iPr—P—PtA4 in example 10, (S)-iPr-LA20 was used to replace (S)-iPr-LA4 to obtain the product as a yellow solid 3.0 g in 47% yield. ¹H NMR (500 MHz, DMSO-d₆): δ(ppm) 0.72 (d J=5.5 Hz, 3H), 0.74 (d J=7.0 Hz, 3H), 1.35 (s, 9H), 2.14-2.19 (m, 1H), 4.62 (dd, J=12.5, 3.0 Hz, 1H), 4.71-4.73 (m, 1H), 4.89 (d, J=12.0 Hz, 1H), 6.93 (d, J=8.0 Hz, 1H), 6.99 (d, J=8.0 Hz, 1H), 7.26-7.29 (m, 3H), 7.36 (t, J=8.5 Hz, 1H), 7.40 (t, J=7.0 Hz, 1H), 7.49-7.52 (m, 1H), 7.63 (d, J=7.5 Hz, 1H), 7.82 (d, J=8.0 Hz, 1H), 7.90 (d, J=8.0 Hz, 1H), 8.06 (d, J=8.5 Hz, 1H), 8.16-8.17 (m, 1H), 9.70 (d, J=6.0 Hz, 1H).

### Example 52

The synthetic route for (R)-iPr-M-PtA21 is as follows:

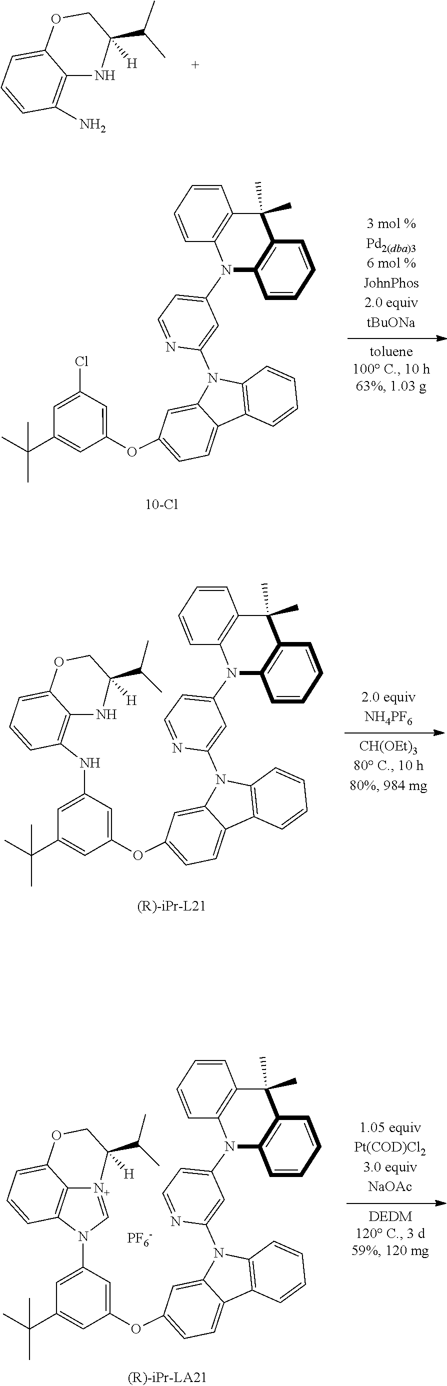

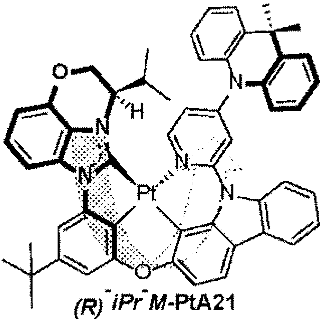

(1) Synthesis of (R)-iPr-LA21: following the synthesis procedure of (R)-iPr-LA4 in example 9, 10-Cl was used to replace 1-Cl to obtain the product as a yellow solid 984 mg in 80% yield. ¹H NMR (500 MHz, DMSO-d₆): δ(ppm) 1.03 (dd, J=9.0, 7.0 Hz, 6H), 1.35 (s, 9H), 1.47 (s, 6H), 2.34-2.41 (m, 1H), 4.50 (dd, J=12.0, 3.0 Hz, 1H), 4.73-4.77 (m, 1H), 4.78 (dd, J=12.0, 3.5 Hz, 1H), 7.16-7.21 (m, 5H), 7.24 (td, J=7.5, 1.5 Hz, 2H), 7.32-7.36 (m, 5H), 7.44-7.48 (m, 3H), 7.50 (t, J=8.0 Hz, 1H), 7.54 (d, J=1.5 Hz, 1H), 7.56 (t, J=2.0 Hz, 2H), 7.69 (d, J=2.0 Hz, 1H), 7.79 (d, J=8.0 Hz, 1H), 8.22 (d, J=7.0 Hz, 1H), 8.30 (d, J=8.5 Hz, 1H), 8.56 (d, J=6.0 Hz, 1H), 10.20 (s, 1H).
(2) Synthesis of (R)-iPr-M-PtA21: following the synthesis procedure of (R)-iPr-M-PtA4 in example 9, (R)-iPr-LA21 was used to replace (R)-iPr-LA4 to obtain the product as a yellow solid 120 mg in 59% yield. ¹H NMR (500 MHz, DMSO-d₆): δ(ppm) 0.83 (t, J=7.0 Hz, 6H), 1.36 (s, 6H), 1.43 (s, 9H), 2.20-2.30 (m, 1H), 4.64 (dd, J=14.0, 3.0 Hz, 1H), 4.79 (d, J=4.0 Hz, 1H), 4.93 (d, J=11.0 Hz, 1H), 6.93 (d, J=8.0 Hz, 1H), 6.97 (d, J=1.5 Hz, 1H), 7.08 (dd, J=7.0, 2.5 Hz, 1H), 7.22 (d, J=8.5 Hz, 1H), 7.32-7.43 (m, 5H), 7.47-7.450 (m, 3H), 7.57 (dd, J=8.0, 1.5 Hz, 2H), 7.71 (d, J=8.0 Hz, 1H), 7.76 (d, J=3.0 Hz, 1H), 7.81 (d, J=8.5 Hz, 1H), 7.90 (d, J=8.0 Hz, 1H), 8.07 (dd, J=7.5, 1.5 Hz, 1H), 9.44 (d, J=7.0 Hz, 1H).

### Example 53

The synthetic route for (S)-iPr-M-PtA21 is as follows:

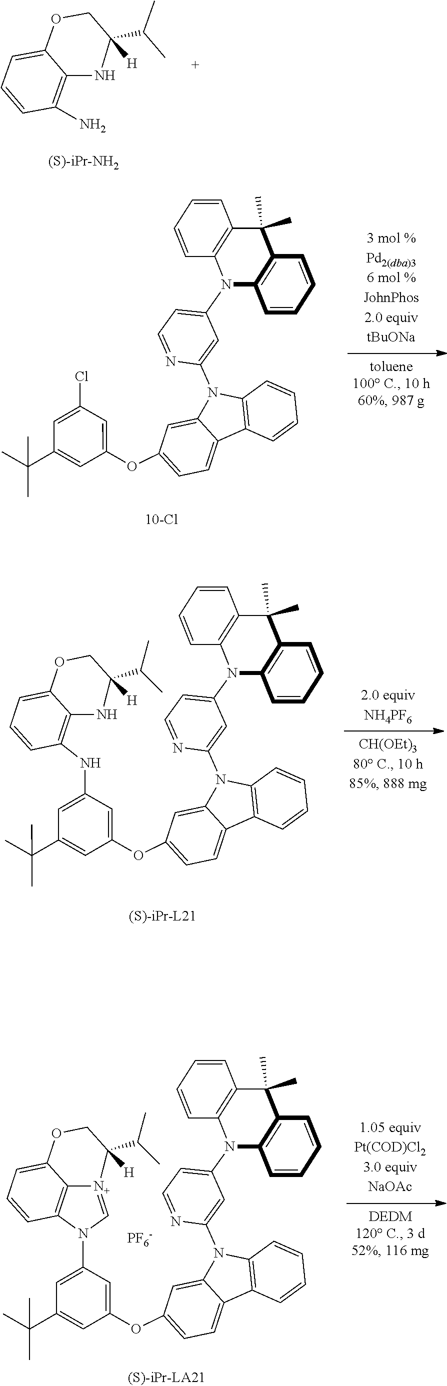

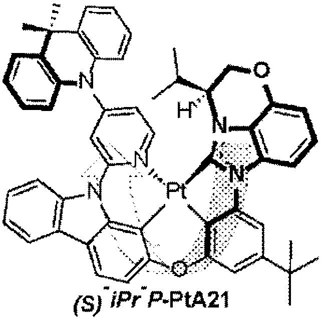

(1) Synthesis of (S)-iPr-LA21: following the synthesis procedure of (S-iPr-LA4 in example 10, 10-Cl was used to replace 1-Cl to obtain the product as a yellow solid 888 mg in 85% yield. ¹H NMR (500 MHz, DMSO-d₆): δ(ppm) 1.03 (dd, J=9.0, 6.5 Hz, 6H), 1.35 (s, 9H), 1.47 (s, 6H), 2.34-2.40 (m, 1H), 4.50 (dd, J=12.0, 3.0 Hz, 1H), 4.72-4.75 (m, 1H), 4.78 (dd, J=12.0, 3.5 Hz, 1H), 7.16-7.25 (m, 5H), 7.24 (td, J=7.5, 1.5 Hz, 2H), 7.32-7.36 (m, 5H), 7.44-7.48 (m, 3H), 7.50 (t, J=8.0 Hz, 1H), 7.55 (d, J=1.5 Hz, 1H), 7.56 (t, J=2.0 Hz, 2H), 7.69 (d, J=2.5 Hz, 1H), 7.79 (d, J=8.5 Hz, 1H), 8.22 (d, J=7.5 Hz, 1H), 8.30 (d, J=8.5 Hz, 1H), 8.56 (d, J=6.0 Hz, 1H), 10.20 (s, 1H).
(2) Synthesis of (S)-iPr—P—PtA21: following the synthesis procedure of (S)-iPr—P—PtA4 in example 10, (S)-iPr-LA21 was used to replace (S)-iPr-LA4 to obtain the product as a yellow solid 116 mg in 52% yield. ¹H NMR (500 MHz, DMSO-d₆): δ(ppm) 0.83 (t, J=6.5 Hz, 6H), 1.36 (s, 6H), 1.43 (s, 9H), 2.20-2.30 (m, 1H), 4.64 (dd, J=12.5, 3.0 Hz, 1H), 4.79 (d, J=4.0 Hz, 1H), 4.93 (d, J=11.5 Hz, 1H), 6.93 (d, J=8.0 Hz, 1H), 6.97 (d, J=1.5 Hz, 1H), 7.09 (dd, J=8.0, 3.0 Hz, 1H), 7.22 (d, J=8.0 Hz, 1H), 7.31-7.43 (m, 5H), 7.48 (td, J=7.5, 1.5 Hz, 3H), 7.57 (dd, J=8.0, 1.5 Hz, 3H), 7.72 (d, J=8.5 Hz, 1H), 7.77 (d, J=3.0 Hz, 1H), 7.81 (d, J=8.0 Hz, 1H), 7.90 (d, J=8.0 Hz, 1H), 8.07 (dd, J=7.5, 1.5 Hz, 1H), 9.43 (d, J=7.0 Hz, 1H).

### Example 54

The synthetic route for (R)-iPr-M-PtA22 is as follows:

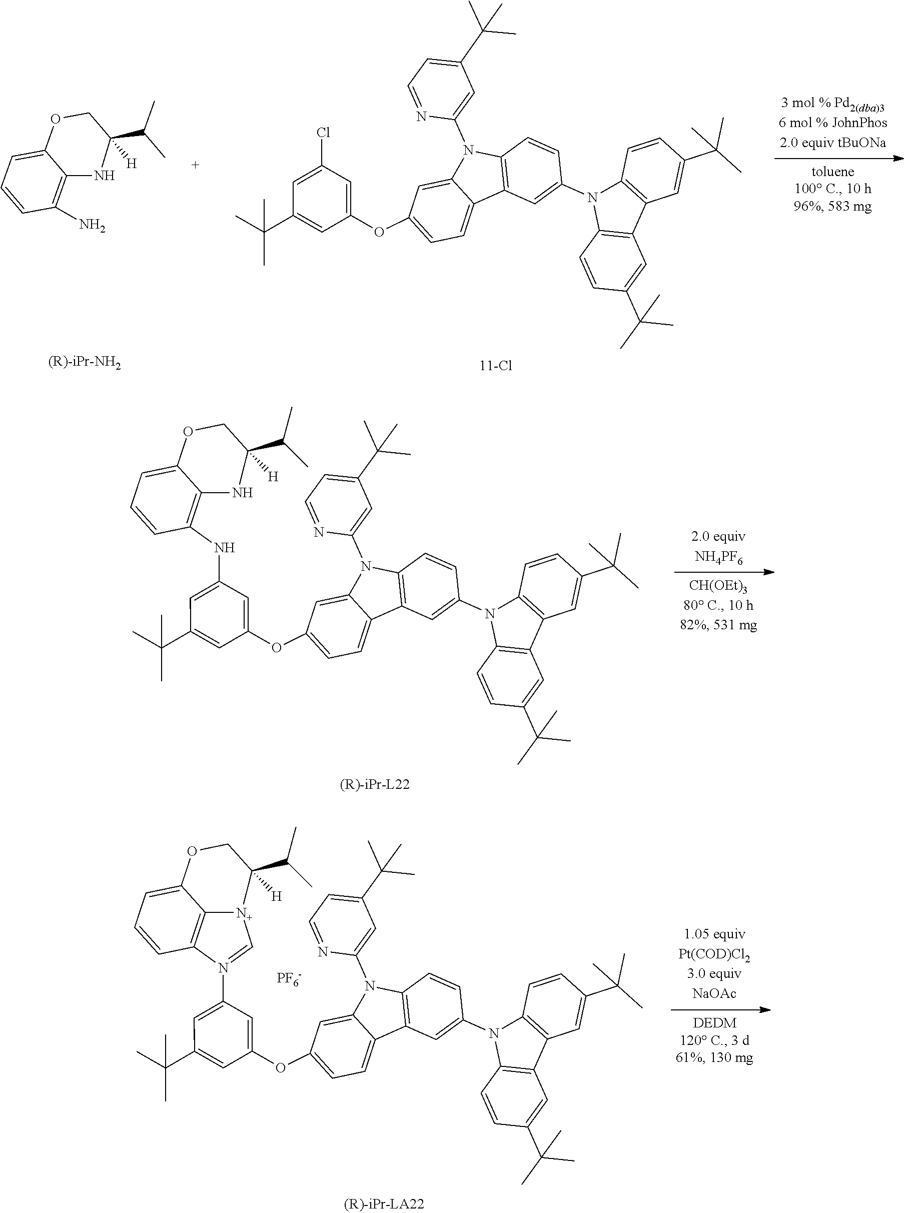

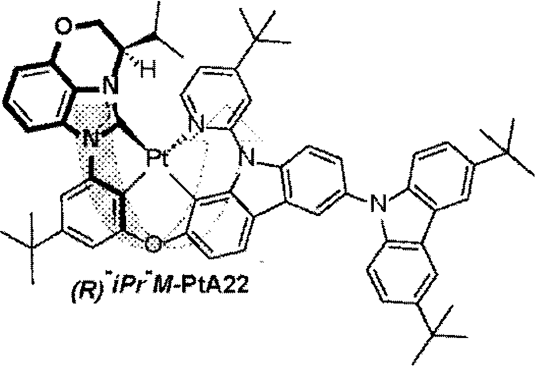

(1) Synthesis of (R)-iPr-LA22: following the synthesis procedure of (R)-iPr-LA4 in example 9, 11-Cl was used to replace 1-Cl to obtain the product as a yellow solid 531 mg in 82% yield. ¹H NMR (500 MHz, DMSO-d₆): δ(ppm) 1.05 (dd, J=8.5, 6.5 Hz, 6H), 1.32 (s, 9H), 1.37 (s, 9H), 1.43 (s, 18H), 2.37-2.43 (m, 1H), 4.52 (dd, J=12.5, 3.0 Hz, 1H), 4.75-4.77 (m, 1H), 4.80 (dd, J=5=12.0, 3.5 Hz, 1H), 7.20-7.23 (m, 2H), 7.29 (s, 1H), 7.31 (t, J=2.0, 2H), 7.36 (d, J=8.5 Hz, 1H), 7.47 (d, J=2.0 Hz, 1H), 7.49-7.50 (m, 2H), 7.51-7.57 (m, 3H), 7.61 (t, J=2.0 Hz, 1H), 7.63 (dd, J=9.0, 2.5 Hz, 1H), 7.79 (d, J=2.0 Hz, 1H), 7.98 (d, J=9.0 Hz, 1H), 8.32 (d, J=2.0 Hz, 2H), 8.43 (d, J=8.5 Hz, 1H), 8.52 (d, J=2.5 Hz, 1H), 8.63 (d, J=6.0 Hz, 1H), 10.24 (s, 1H).
(2) Synthesis of (R)-iPr-M-PtA22: following the synthesis procedure of (R)-iPr-M-PtA4 in example 9, (R)-iP-LA22 was used to replace (R)-iPr-LA4 to obtain the product as a yellow solid 130 mg in 61% yield. ¹H NMR (500 MHz, DMSO-d₆): δ(ppm) 0.74 (dd, J=7.0, 4.5 Hz, 6H), 1.33 (s, 9H), 1.44 (s, 18H), 1.46 (s, 9H), 2.16-2.23 (m, 1H), 4.52 (d, J=10.0 Hz, 1H), 4.72 (d, J=5.0 Hz, 1H), 4.88 (d, J=11.5 Hz, 1H), 6.94 (d, J=8.0 Hz, 1H), 7.03 (d, J=1.5 Hz, 1H), 7.24 (d, J=6.5 Hz, 1H), 7.30 (d, J=8.0 Hz, 1H), 7.38-7.42 (m, 3H), 7.48 (s, 1H), 7.51 (dd, J=8.5, 2.0 Hz, 2H), 7.64 (dd, J=9.0, 2.5 Hz, 1H), 7.69 (d, J=8.5 Hz, 1H), 8.00 (d, J=8.5 Hz, 1H), 8.22 (d, J=2.0 Hz, 1H), 8.28 (d, J=8.5 Hz, 1H), 8.33 (d, J=2.5 Hz, 2H), 8.42 (d, J=2.5 Hz, 1H), 9.78 (d, J=6.5 Hz, 1H).

### Example 55

The synthetic route for (S)-iPr—P—PtA22 is as follows:

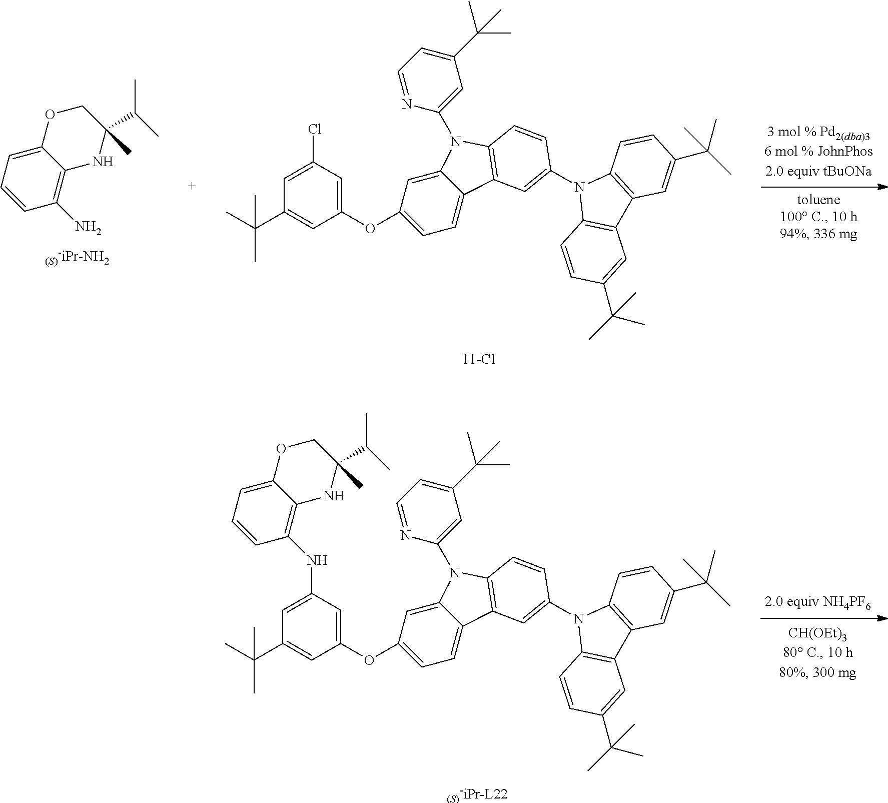

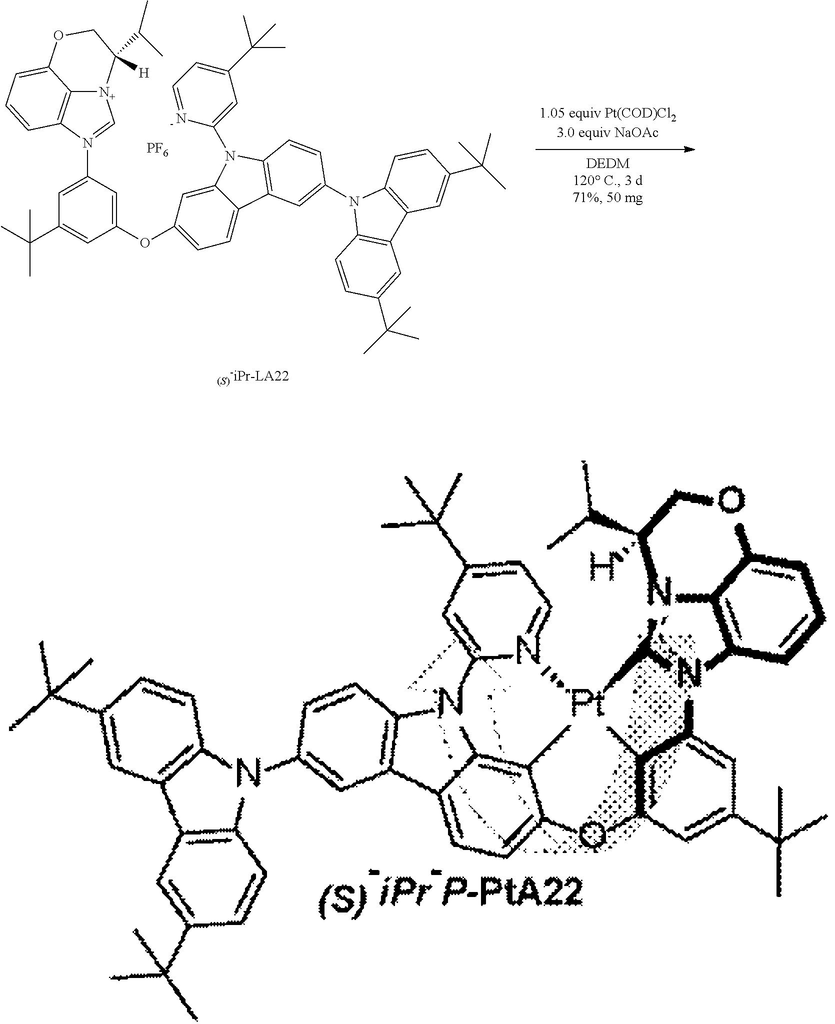

(1) Synthesis of (S)-iP-LA22: following the synthesis procedure of (S)-iPr-LA4 in example 10, 11-Cl was used to replace 1-Cl to obtain the product as a yellow solid 300 mg in 80% yield. ¹H NMR (500 MHz, DMSO-d₆): δ(ppm) 1.05 (dd, J=8.5, 6.0 Hz, 6H), 1.32 (s, 9H), 1.37 (s, 9H), 1.43 (s, 18H), 2.37-2.43 (m, 1H), 4.52 (dd, J=12.0, 3.0 Hz, 1H), 4.75-4.77 (m, 1H), 4.80 (dd, J=12.0, 3.5 Hz, 1H), 7.20-7.23 (m, 2H), 7.29 (s, 1H), 7.30-7.31 (m, 2H), 7.36 (d, J=8.5 Hz, 1H), 7.48 (d, J=2.0 Hz, 1H), 7.49-7.50 (m, 2H), 7.52-7.57 (m, 3H), 7.60 (t, J=1.5 Hz, 1H), 7.64 (dd, J=8.5, 2.0 Hz, 1H), 7.79 (d, J=2.5 Hz, 1H), 7.98 (d, J=9.0 Hz, 1H), 8.32 (d, J=2.5 Hz, 2H), 8.43 (d, J=8.5 Hz, 1H), 8.52 (d, J=2.5 Hz, 1H), 8.63 (d, J=5.5 Hz, 1H), 10.23 (s, 1H).
(2) Synthesis of (S)-iPr—P—PtA21: following the synthesis procedure of (S)-iPr—P—PtA4 in example 10, (S)-iPr-LA21 was used to replace (S)-iPr-LA4 to obtain the product as a yellow solid 150 mg in 71% yield. ¹H NMR (500 MHz, DMSO-d₆): δ(ppm) 0.74 (dd, J=7.0, 5.0 Hz, 6H), 1.34 (s, 9H), 1.44 (s, 18H), 1.46 (s, 9H), 2.17-2.23 (m, 1H), 4.54 (d, J=10.5 Hz, 1H), 4.73 (d, J=5.5 Hz, 1H), 4.89 (d, J=11.5 Hz, 1H), 6.95 (d, J=8.0 Hz, 1H), 7.03 (d, J=1.5 Hz, 1H), 7.26 (dd, J=6.5, 2.0 Hz, 1H), 7.30 (d, J=8.0 Hz, 1H), 7.39-7.42 (m, 3H), 7.49 (d, J=2.0, 1H), 7.51 (d, J=3.0 Hz, 1H), 7.52 (d, J=2.0 Hz, 1H), 7.65 (dd, J=8.5, 2.0 Hz, 1H), 7.70 (d, J=8.5 Hz, 1H), 8.00 (d, J=8.5 Hz, 1H), 8.23 (d, J=2.0 Hz, 1H), 8.29 (d, J=9.0 Hz, 1H), 8.33 (d, J=2.0 Hz, 2H), 8.42 (d, J=2.0 Hz, 1H), 9.77 (d, J=6.0 Hz, 1H).

### Example 56

The synthetic route for (R)-iBu-M-PtA23 is as follows:

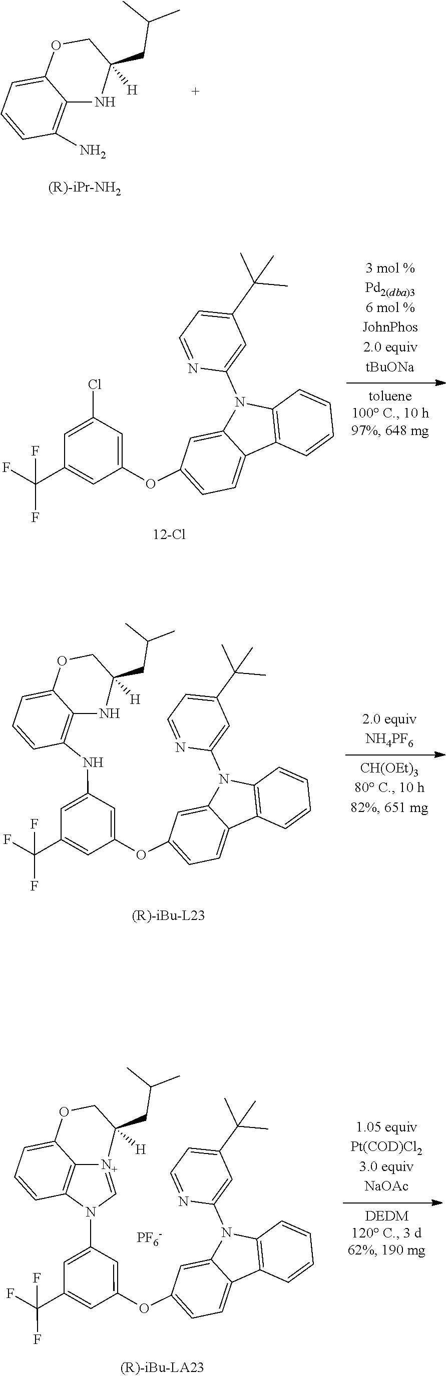

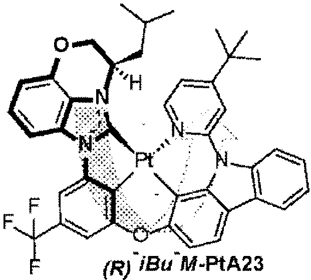

(1) Synthesis of (R)-iBu-LA23: following the synthesis procedure of (R)-iBu-LA6 in example 12, 12-Cl was used to replace 1-Cl to obtain the product as a yellow solid 651 mg in 82% yield. ¹H NMR (500 MHz, DMSO-d₆): δ(ppm) 0.97 (d, J=6.5 Hz, 3H), 1.01 (d, J=6.5 Hz, 3H), 1.32 (s, 9H), 1.72-1.77 (m, 1H), 1.79-1.87 (m, 1H), 1.92-1.97 (m, 1H), 4.50 (dd, J=12.0, 5.5 Hz, 1H), 4.66 (dd, J=12.5, 3.0 Hz, 1H), 4.93-4.98 (m, 1H), 7.21 (d, J=8.0 Hz, 1H), 7.27 (dd, J=8.0, 2.0 Hz, 1H), 7.34-7.37 (m, 1H), 7.42 (d, J=8.5 Hz, 1H), 7.47-7.50 (m, 2H), 7.55 (t, J=8.0 Hz, 1H), 7.61 (d, J=2.5 Hz, 1H), 7.70 (d, J=1.5 Hz, 1H), 7.74-7.78 (m, 3H), 7.97 (s, 1H), 8.27 (d, J=7.5 Hz, 1H), 8.37 (d, J=7.5 Hz, 1H), 8.60 (d, J=5.0 Hz, 1H), 10.27 (s, 1H).
(2) Synthesis of (R)-iBu-M-PtA23: following the synthesis procedure of (R)-iBu-M-PtA6 in example 12, (R)-iBu-LA23 was used to replace (R)-iBu-LA6 to obtain the product as a yellow solid 190 mg in 62% yield. ¹H NMR (500 MHz, DMSO-d₆): δ(ppm) 0.31 (d, J=6.5 Hz, 3H), 0.54 (d, J=6.5 Hz, 3H), 1.14 (s, 9H), 1.20-1.27 (m, 1H), 1.30-1.37 (m, 1H), 1.58-1.64 (m, 1H), 1.75-1.82 (m, 1H), 4.41 (d, J=11.0 Hz, 1H), 4.72 (d, J=11.0 Hz, 1H), 4.83 (t, J=8.0 Hz, 1H), 6.95-6.98 (m, 2H), 7.24 (s, 1H), 7.34 (d, J=8.5 Hz, 1H), 7.38-7.43 (m, 2H), 7.47 (s, 1H), 7.49-7.52 (m, 1H), 7.67 (d, J=8.0 Hz, 1H), 7.86 (d, J=8.5 Hz, 1H), 7.96 (d, J=8.5 Hz, 1H), 7.98 (d, J=2.0 Hz, 1H), 8.18 (d, J=7.0 Hz, 1H), 9.61 (d, J=6.0 Hz, 1H).

### Example 57

The synthetic route for (S)-iBu-P—PtA23 is as follows:

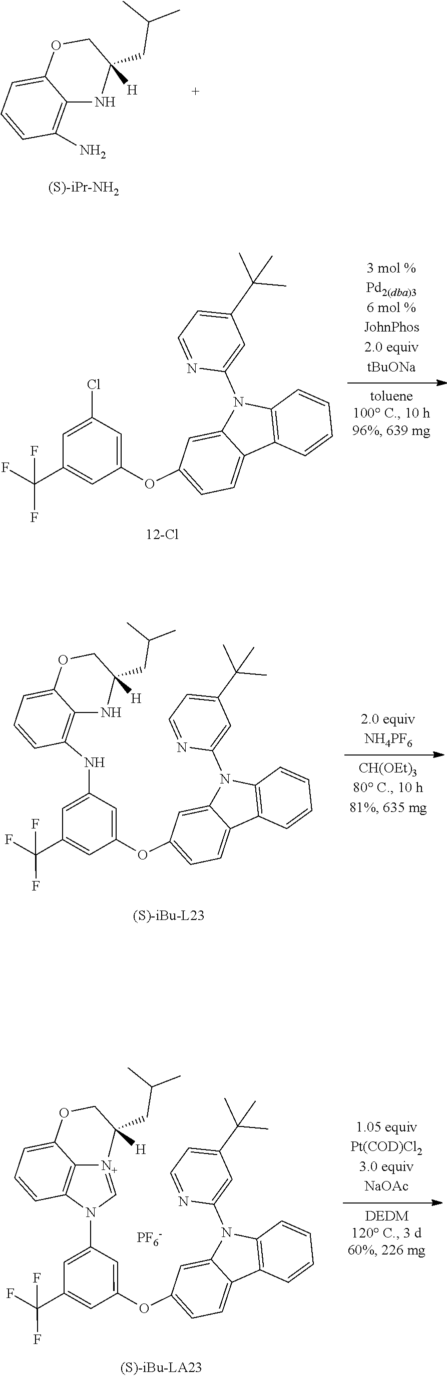

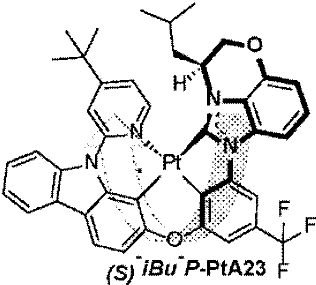

(1) Synthesis of (S)-iBu-LA23: following the synthesis procedure of (S)-iBu-LA6 in example 13, 12-Cl was used to replace 1-Cl to obtain the product as a yellow solid 635 mg in 81% yield.

¹H NMR (500 MHz, DMSO-d₆): δ(ppm) 0.96 (d, J=6.5 Hz, 3H), 1.01 (d, J=6.5 Hz, 3H), 1.32 (s, 9H), 1.71-1.77 (m, 1H), 1.79-1.87 (m, 1H), 1.92-1.97 (m, 1H), 4.50 (dd, J=12.0, 5.5 Hz, 1H), 4.66 (dd, J=12.0, 3.0 Hz, 1H), 4.93-4.97 (m, 1H), 7.21 (d, J=8.0 Hz, 1H), 7.27 (dd, J=8.5, 2.5 Hz, 1H), 7.34-7.37 (m, 1H), 7.42 (d, J=8.5 Hz, 1H), 7.47-7.50 (m, 2H), 7.55 (t, J=8.0 Hz, 1H), 7.61 (d, J=2.0 Hz, 1H), 7.70 (d, J=1.5 Hz, 1H), 7.74-7.78 (m, 3H), 7.97 (s, 1H), 8.27 (d, J=8.0 Hz, 1H), 8.37 (d, J=8.5 Hz, 1H), 8.60 (d, J=5.5 Hz, 1H), 10.26 (s, 1H).
(2) Synthesis of (S)-iBu-P—PtA23: following the synthesis procedure of (S)-iBu-P—PtA6 in example 13, (S)-iBu-LA23 was used to replace (S)-iBu-LA6 to obtain the product as a yellow solid 266 mg in 60% yield. ¹H NMR (500 MHz, DMSO-d₆): δ(ppm) 0.34 (d, J=6.5 Hz, 3H), 0.56 (d, J=6.5 Hz, 3H), 1.20 (s, 9H), 0.80-0.87 (m, 1H), 1.33-1.39 (m, 1H), 1.61-1.66 (m, 1H), 1.77-1.83 (m, 1H), 4.49 (d, J=11.0 Hz, 1H), 4.73 (d, J=11.5 Hz, 1H), 4.87 (t, J=8.0 Hz, 1H), 6.97 (d, J=8.0 Hz, 1H), 7.09 (d, J=5.5 Hz, 1H), 7.26 (s, 1H), 7.33 (d, J=8.5 Hz, 1H), 7.39-7.43 (m, 2H), 7.50-7.55 (m, 1H), 7.55 (s, 1H), 7.71 (d, J=8.0 Hz, 1H), 7.89 (d, J=8.0 Hz, 1H), 7.95 (d, J=8.0 Hz, 1H), 8.04 (d, J=2.0 Hz, 1H), 8.19 (d, J=7.5 Hz, 1H), 9.63 (d, J=6.5 Hz, 1H).

### Example 58

The synthetic route for (R)-iBu-M-PtA24 is as follows:

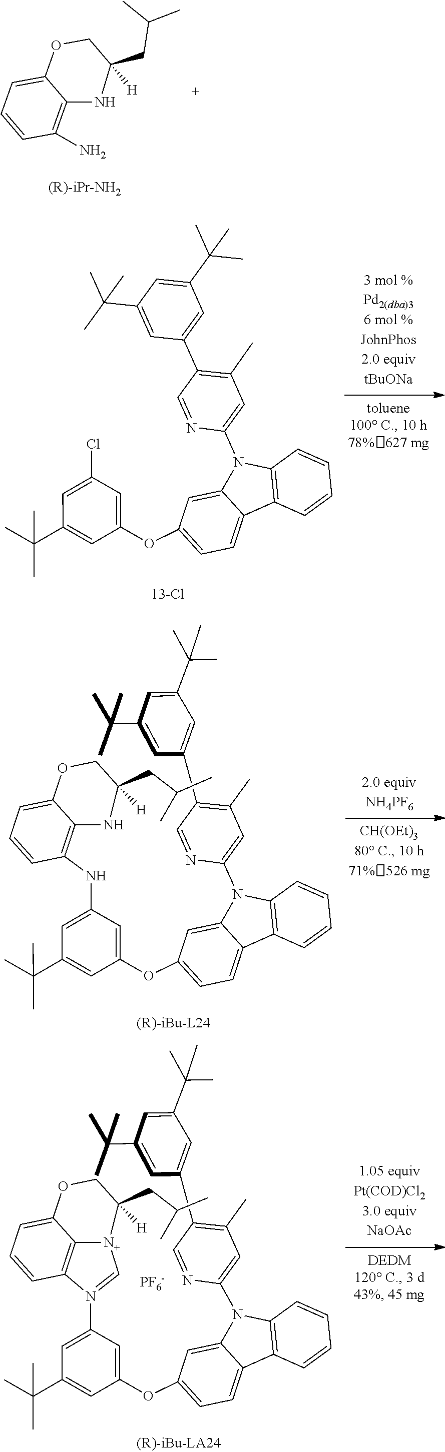

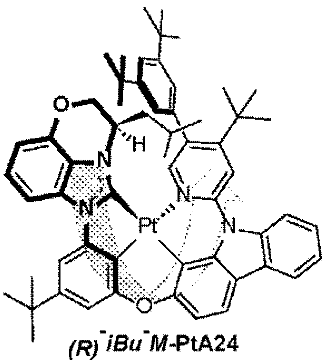

(1) Synthesis of (R)-iBu-LA24: following the synthesis procedure of (R)-iBu-LA6 in example 12, 13-Cl was used to replace 1-Cl to obtain the product as a yellow solid 45 mg in 43% yield.
(2) Synthesis of (R)-iBu-M-PtA24: following the synthesis procedure of (R)-iBu-M-PtA6 in example 12, (R)-iBu-LA24 was used to replace (R)-iBu-LA6 to obtain the product as a yellow solid 45 mg in 43% yield. ¹H NMR (500 MHz, DMSO-d₆): δ(ppm) 0.51 (d, J=7.0 Hz, 3H), 0.65 (d, J=6.5 Hz, 3H), 1.29 (s, 18H), 1.43 (s, 9H), 1.44-1.52 (m, 1H), 1.46-1.51 (m, 1H), 1.61-1.67 (m, 1H), 1.92-1.97 (m, 1H), 2.25 (s, 3H), 4.20 (dd, J=11.5, 2.5 Hz 1H), 4.55 (d, J=11.5 Hz, 1H), 4.86 (t, J=7.5 Hz, 1H), 6.89 (d, J=8.0 Hz, 1H), 6.99 (d, J=1.5 Hz, 1H), 7.17 (d, J=2.0 Hz, 2H), 7.28 (d, J=8.5 Hz, 1H), 7.34 (t, J=8.0 Hz, 1H), 7.40-7.43 (m, 1H), 7.47-7.53 (m, 3H), 7.71 (d, J=8.5 Hz, 1H), 7.90 (d, J=8.5 Hz, 1H), 8.03 (d, J=8.0 Hz, 1H), 8.16-8.18 (m, 2H), 9.42 (s, 1H).

### Example 59

The synthetic route for (S)-iBu-P—PtA24 is as follows:

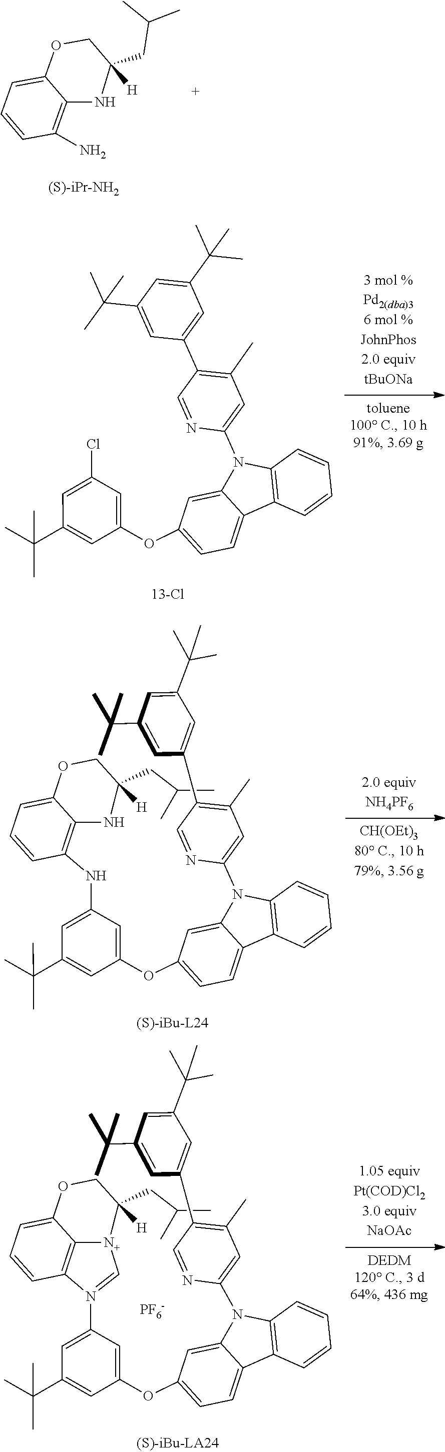

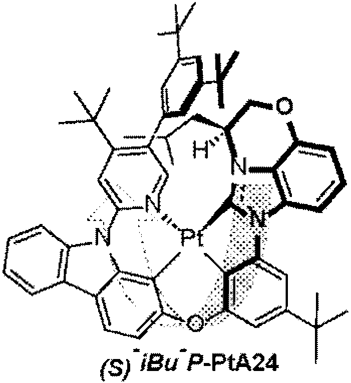

(1) Synthesis of (S)-iBu-LA24: following the synthesis procedure of (S)-iBu-LA6 in example 13, 13-Cl was used to replace 1-Cl to obtain the product as a yellow solid 3.56 g in 79% yield. ¹H NMR (500 MHz, DMSO-d₆): δ(ppm) 0.96 (d, J=6.5 Hz, 3H), 1.00 (d, J=6.5 Hz, 3H), 1.34 (s, 18H), 1.36 (s, 9H), 1.71-1.77 (m, 1H), 1.79-1.87 (m, 1H), 1.93-1.99 (m, 1H), 2.40 (s, 3H), 4.48 (dd, J=12.0, 5.5 Hz, 1H), 4.66 (dd, J=12.0, 3.0 Hz, 1H), 4.89-4.93 (m, 1H), 7.18-7.22 (m, 3H), 7.29 (d, J=1.5 Hz, 2H), 7.34-7.37 (m, 2H), 7.47-7.55 (m, 5H), 7.70 (d, J=2.0 Hz, 1H), 7.74 (s, 1H), 7.85 (d, J=8.5 Hz, 1H), 8.26 (d, J=7.5 Hz, 1H), 8.34 (d, J=8.5 Hz, 1H), 8.50 (s, 1H), 10.18 (s, 1H).
(2) Synthesis of (S)-iBu-P—PtA24: following the synthesis procedure of (S)-iBu-P—PtA6 in example 13, (S)-iBu-LA24 was used to replace (S)-iBu-LA6 to obtain the product as a yellow solid 436 mg in 64% yield. ¹H NMR (500 MHz, DMSO-d₆): δ(ppm) 0.50 (d, J=7.0 Hz, 3H), 0.65 (d, J=6.0 Hz, 3H), 1.29 (s, 18H), 1.42 (s, 9H), 1.45-1.50 (m, 1H), 1.61-1.66 (m, 1H), 1.91-1.95 (m, 1H), 2.25 (s, 3H), 4.19 (dd, J=11.5, 2.0 Hz 1H), 4.54 (d, J=11.0 Hz, 1H), 4.85 (t, J=7.0 Hz, 1H), 6.89 (d, J=8.0 Hz, 1H), 6.99 (d, J=1.5 Hz, 1H), 7.17 (d, J=1.5 Hz, 2H), 7.28 (d, J=8.5 Hz, 1H), 7.34 (t, J=8.0 Hz, 1H), 7.40-7.43 (m, 1H), 7.47-7.52 (m, 3H), 7.70 (d, J=8.5 Hz, 1H), 7.90 (d, J=8.5 Hz, 1H), 8.02 (d, J=8.0 Hz, 1H), 8.15-8.17 (m, 2H), 9.41 (s, 1H).

### Example 60

The synthetic route for (R,S)-Tjy-M-PtA25 is as follows:

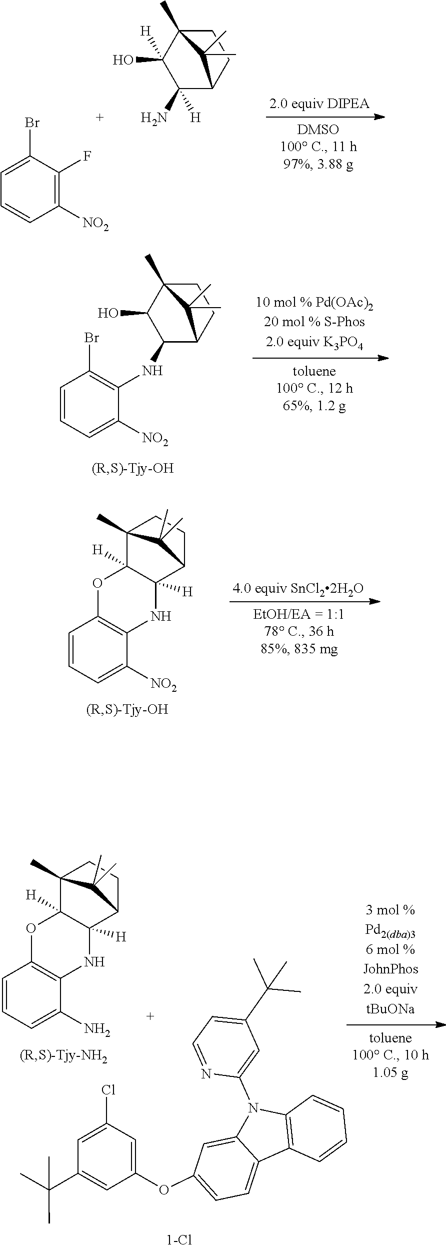

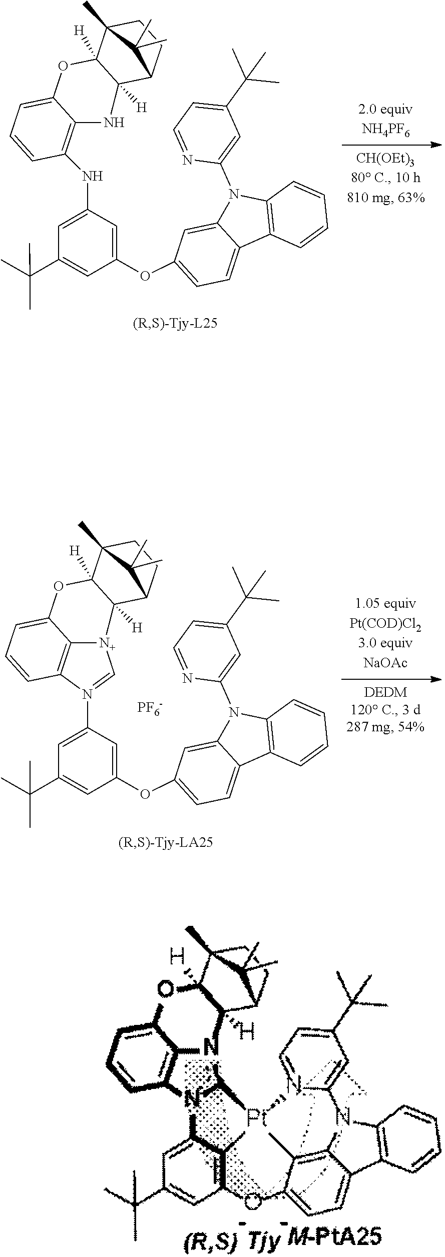

(1) Synthesis of (R,S)-Tjy-LA25: following the synthesis procedure of (R,S)-LA1 in example 1, the intermediate

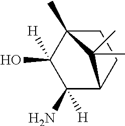

was used to replace

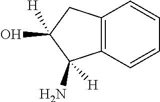 [SMILES: N[C@@H]1c2ccccc2C[C@@H]1O]

to obtain the product (R,S)-Tjy-LA25 as a yellow foamy solid 810 mg in 63% yield. ¹H NMR (500 MHz, DMSO-d₆): δ(ppm) 0.51 (s, 3H), 0.90 (s, 3H), 1.19 (s, 3H), 1.30-1.34 (m, 1H), 1.38 (d, J=1.0 Hz, 18H), 1.65-1.76 (m, 2H), 1.97-2.05 (m, 1H), 2.57 (d, J=4.0 Hz, 1H), 4.56 (d, J=7.0 Hz, 1H), 4.90 (d, J=7.0 Hz, 1H), 6.99-7.03 (m, 2H), 7.11 (dd, J=8.5, 2.0 Hz, 1H), 7.22 (d, J=8.5 Hz, 1H), 7.31-7.35 (m, 2H), 7.38 (t, J=2.0 Hz, 1H), 7.42-7.47 (m, 2H), 7.49 (t, J=2.0 Hz, 1H), 7.60 (d, J=1.5 Hz, 1H), 7.63 (d, J=2.0 Hz, 1H), 7.68 (d, J=8.0 Hz, 1H), 8.10 (d, J=7.5 Hz, 1H), 8.13 (d, J=8.5 Hz, 1H), 8.57 (d, J=5.0 Hz, 1H), 9.47 (s, 1H).
(2) Synthesis of (R,S)-Tjy-M-PtA25: following the synthesis procedure of (R,S)-M-PtA1 in example 1, (R,S)-Tjy-LA25 was used to replace (R,S)-LA1 to obtain the product (R,S)-Tjy-M-PtA25 as a yellow solid 287 mg in 54% yield. ¹H NMR (500 MHz, CDCl₃): δ(ppm) 0.29 (s, 3H), 0.44-0.52 (m, 1H), 0.64 (s, 3H), 1.11 (s, 4H), 1.23 (s, 9H), 1.30-1.45 (m, 2H), 1.49 (s, 9H), 2.70 (d, J=4.0 Hz, 1H), 4.39 (d, J=7.5 Hz, 1H), 4.64 (d, J=7.5 Hz, 1H), 6.73 (d, J=8.0 Hz, 1H), 6.87 (dd, J=6.0, 2.0 Hz, 1H), 7.14-7.25 (m, 2H), 7.33-7.43 (m, 4H), 7.48 (d, J=2.0 Hz, 1H), 7.81-7.84 (m, 4H), 8.03 (dd, J=7.0, 1.0 Hz, 1H), 8.12 (d, J=2.0 Hz, 1H), 9.62 (d, J=6.5 Hz, 1H).

### Example 61

The synthetic route for (S,R)-Tjy-P—PtA25 is as follows:

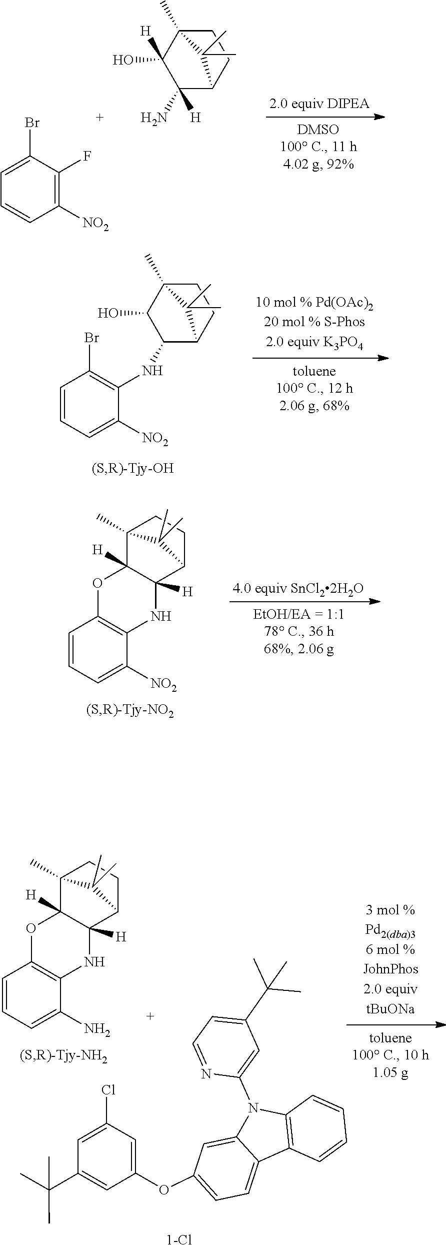

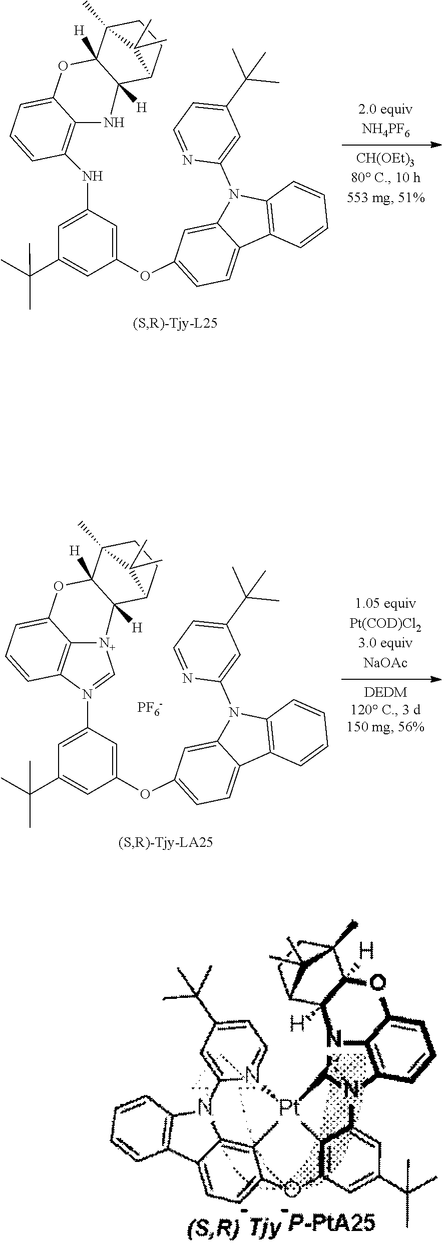

(1) Synthesis of (S,R)-Tjy-LA26: following the synthesis procedure of (S,R)-LA1 in example 2, the intermediate

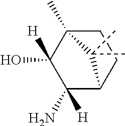

was used to replace

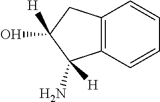 [SMILES: N[C@H]1c2ccccc2C[C@H]1O]

to obtain the product as a yellow foamy solid 553 mg in 51% yield. ¹H NMR (500 MHz, DMSO-d₆): δ(ppm) 0.51 (s, 3H), 0.90 (s, 3H), 1.19 (s, 3H), 1.31-1.34 (m, 1H), 1.38 (s, 18H), 1.64-1.78 (m, 2H), 1.97-2.03 (m, 1H), 2.57 (d, J=4.5 Hz, 1H), 4.56 (d, J=7.0 Hz, 1H), 4.90 (d, J=7.0 Hz, 1H), 6.99-7.03 (m, 2H), 7.11 (dd, J=8.5, 2.0 Hz, 1H), 7.22 (d, J=8.0 Hz, 1H), 7.30-7.36 (m, 2H), 7.39 (t, J=2.0 Hz, 1H), 7.41-7.47 (m, 2H), 7.49 (t, J=2.0 Hz, 1H), 7.59 (d, J=2.5 Hz, 1H), 7.64 (d, J=2.0 Hz, 1H), 7.69 (d, J=8.5 Hz, 1H), 8.10 (d, J=7.5 Hz, 1H), 8.14 (d, J=8.5 Hz, 1H), 8.57 (d, J=5.5 Hz, 1H), 9.46 (s, 1H).
(2) Synthesis of (S,R)-Tjy-P—PtA26: following the synthesis procedure of (S,R)—P—PtA1 in example 2, (S,R)-Tjy-LA25 was used to replace (S,R)-Tjy-P—PtA26 to obtain the product as a yellow solid 150 mg in 56% yield. ¹H NMR (500 MHz, CDCl₃): δ(ppm) 0.30 (s, 3H), 0.49 (ddd, J=12.7, 9.1, 3.2 Hz, 1H), 0.65 (s, 3H), 1.12 (s, 3H), 1.13-1.17 (m, 1H), 1.25 (s, 9H), 1.32-1.44 (m, 2H), 1.49 (s, 9H), 2.71 (d, J=4.0 Hz, 1H), 4.41 (d, J=7.5 Hz, 1H), 4.66 (d, J=7.0 Hz, 1H), 6.74 (d, J=8.0 Hz, 1H), 6.89 (dd, J=6.5, 2.0 Hz, 1H), 7.19 (d, J=1.5 Hz, 1H), 7.32-7.46 (m, 5H), 7.49 (d, J=1.5 Hz, 1H), 7.82 (d, J=8.5 Hz, 1H), 7.84 (d, J=8.0 Hz, 1H), 8.04 (dd, J=8.0, 1.5 Hz, 1H), 8.14 (d, J=1.5 Hz, 1H), 9.61 (d, J=6.0 Hz, 1H).

### Example 62

The synthetic route for (R,S)-Tjy-M-PtA26 is as follows:

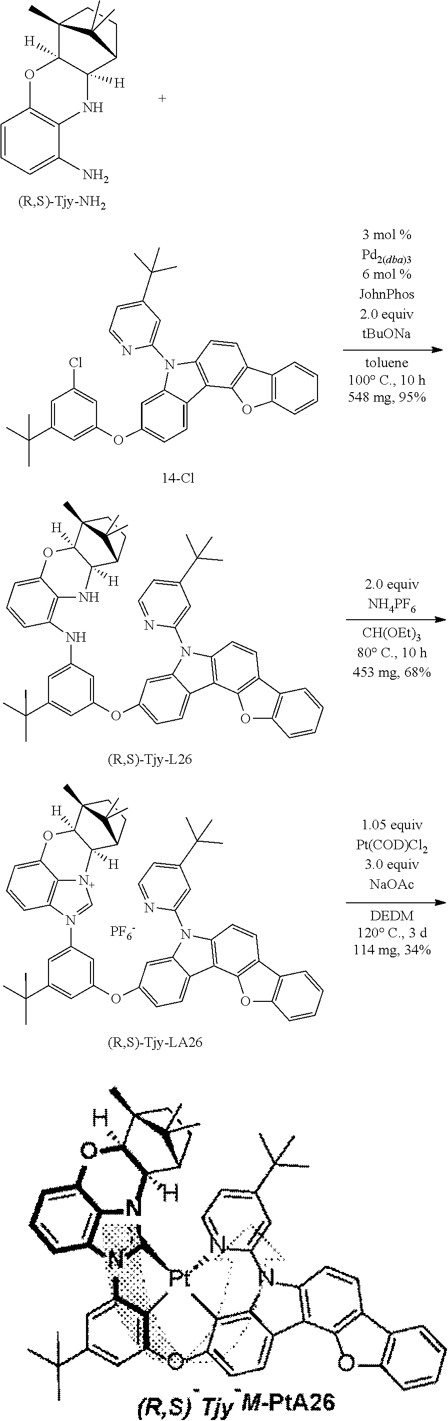

(1) Synthesis of (R,S)-Tjy-LA2: following the synthesis procedure of (R,S)-Tjy-LA25 in example 60, 14-Cl was used to replace 1-Cl to obtain the product as a yellow solid 453 mg in 68% yield. ¹H NMR (500 MHz, CDCl₃): δ(ppm) 0.52 (s, 3H), 0.90 (s, 3H), 1.19 (s, 3H), 1.33 (ddd, J=14.0, 9.0, 5.0 Hz, 1H), 1.41 (s, 9H), 1.64 (ddd, J=13.5, 9.5, 4.0 Hz, 1H), 1.73 (td, J=12.9, 11.9, 3.8 Hz, 1H), 1.96-2.03 (m, 1H), 2.60 (d, J=4.5 Hz, 1H), 4.56 (d, J=7.0 Hz, 1H), 4.87 (d, J=7.5 Hz, 1H), 7.01 (d, J=8.0 Hz, 1H), 7.22-7.25 (m, 2H), 7.30-7.32 (m, 2H), 7.37 (dd, J=5.5, 2.0 Hz, 1H), 7.40 (t, J=7.5 Hz, 1H), 7.45-7.51 (m, 3H), 7.61-7.65 (m, 2H), 7.66-7.69 (m, 2H), 7.74 (d, J=8.0 Hz, 1H), 7.96-8.01 (m, 2H), 8.57 (d, J=8.0 Hz, 1H), 8.61 (d, J=5.0 Hz, 1H), 9.46 (s, 1H).
(2) Synthesis of (R,S)-Tjy-M-PtA26: following the synthesis procedure of (R,S)-Tjy-M-PtA25 in example 60, (R,S)-Tjy-LA25 was used to replace (R,S)-Tjy-M-PtA26 to obtain the product as a yellow solid 114 mg in 34% yield. ¹H NMR (500 MHz, CDCl₃): δ(ppm) 0.30 (s, 3H), 0.42-0.50 (m, 1H), 0.63 (s, 3H), 1.07 (s, 9H), 1.13 (s, 3H), 1.15-1.19 (m, 1H), 1.29-1.37 (m, 1H), 1.53 (dd, J=12.0, 3.5 Hz, 1H), 2.63 (d, J=4.0 Hz, 1H), 4.42 (d, J=7.0 Hz, 1H), 4.48 (d, J=7.0 Hz, 1H), 6.68 (dd, J=6.0, 2.0 Hz, 1H), 6.77 (d, J=8.0 Hz, 1H), 7.06 (d, J=7.5 Hz, 1H), 7.15 (dd, J=8.0, 1.0 Hz, 1H), 7.19-7.23 (m, 1H), 7.28 (d, J=8.0 Hz, 1H), 7.33 (d, J=8.0 Hz, 1H), 7.37-7.41 (m, 1H), 7.47 (ddd, J=8.5, 7.5, 1.5 Hz, 1H), 7.57 (d, J=8.0 Hz, 1H), 7.74 (d, J=8.5 Hz, 1H), 7.78 (d, J=8.5 Hz, 1H), 7.90 (d, J=8.0 Hz, 1H), 7.99 (d, J=7.5 Hz, 1H), 8.05 (d, J=2.0 Hz, 1H), 8.28 (d, J=8.0 Hz, 1H), 9.78 (d, J=6.0 Hz, 1H).

### Example 63

The synthetic route for (S,R)-Tjy-P—PtA26 is as follows:

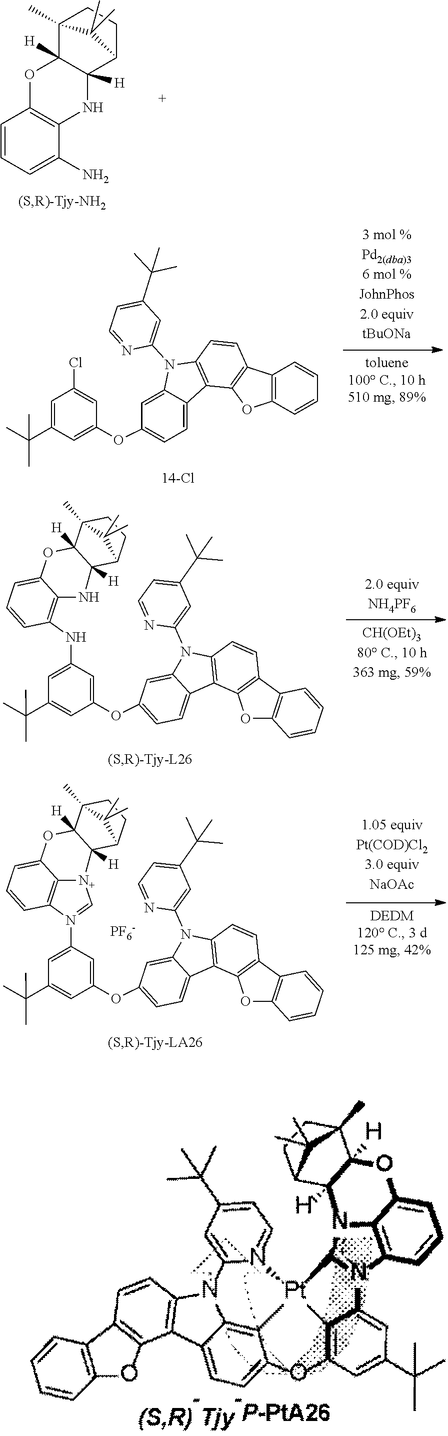

(1) Synthesis of (S,R)-Tjy-LA26: following the synthesis procedure of (S,R)-Tjy-LA25 in example 61, 14-Cl was used to replace 1-Cl to obtain the product as a yellow solid 363 mg in 59% yield. ¹H NMR (500 MHz, CDCl₃): δ(ppm) 0.52 (s, 3H), 0.90 (s, 3H), 1.19 (s, 3H), 1.33 (ddd, J=14.0, 9.0, 5.0 Hz, 1H), 1.40 (s, 9H), 1.65 (ddd, J=13.0, 9.0, 4.0 Hz, 1H), 1.73 (td, J=12.0, 4.0 Hz, 1H), 1.96-2.03 (m, 1H), 2.59 (d, J=4.0 Hz, 1H), 4.56 (d, J=7.5 Hz, 1H), 4.88 (d, J=7.0 Hz, 1H), 7.01 (d, J=8.0 Hz, 1H), 7.22-7.25 (m, 2H), 7.29-7.34 (m, 2H), 7.36-7.42 (m, 2H), 7.45-7.51 (m, 3H), 7.60-7.70 (m, 4H), 7.74 (t, J=8.0 Hz, 1H), 7.96-8.02 (m, 2H), 8.57 (d, J=8.5 Hz, 1H), 8.61 (d, J=5.0 Hz, 1H), 9.46 (s, 1H).
(2) Synthesis of (S,R)-Tjy-P—PtA26: following the synthesis procedure of (S,R)-Tjy-P—PtA25 in example 61, (S,R)-Tjy-LA26 was used to replace (S,R)-Tjy-LA25 to obtain the product as a yellow solid 125 mg in 42% yield. ¹H NMR (500 MHz, CDCl₃): δ(ppm) 0.30 (s, 3H), 0.42-0.50 (m, 1H), 0.63 (s, 3H), 1.07 (s, 9H), 1.13 (s, 3H), 1.15-1.21 (m, 1H), 1.29-1.37 (m, 1H), 1.53 (dd, J=12.0, 3.5 Hz, 1H), 2.63 (d, J=4.0 Hz, 1H), 4.42 (d, J=7.0 Hz, 1H), 4.48 (d, J=7.0 Hz, 1H), 6.68 (d, J=5.5 Hz, 1H), 6.77 (d, J=8.0 Hz, 1H), 7.07 (d, J=7.5 Hz, 1H), 7.15 (dd, J=8.0, 1.0 Hz, 1H), 7.20-7.23 (m, 1H), 7.28 (d, J=8.0 Hz, 1H), 7.33 (d, J=8.0 Hz, 1H), 7.37-7.41 (m, 1H), 7.48 (ddd, J=8.5, 7.0, 1.0 Hz, 1H), 7.57 (d, J=8.5 Hz, 1H), 7.74 (d, J=8.0 Hz, 1H), 7.78 (d, J=8.5 Hz, 1H), 7.90 (d, J=8.5 Hz, 1H), 7.99 (d, J=8.0 Hz, 1H), 8.05 (s, 1H), 8.28 (d, J=8.5 Hz, 1H), 9.78 (d, J=6.0 Hz, 1H).

### Example 64

The synthetic route for (S)-iBu-P—PtA27 is as follows:

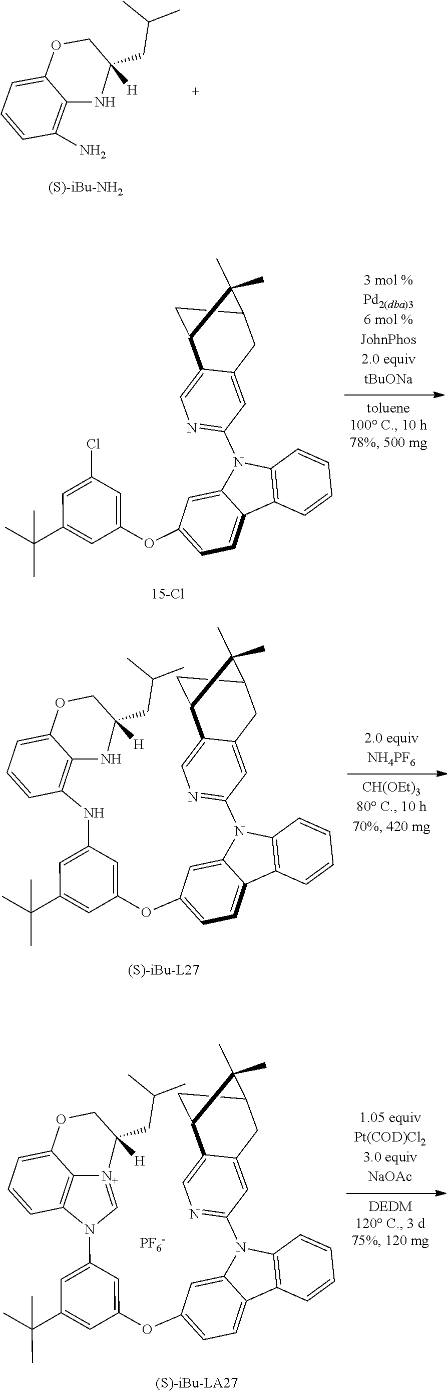

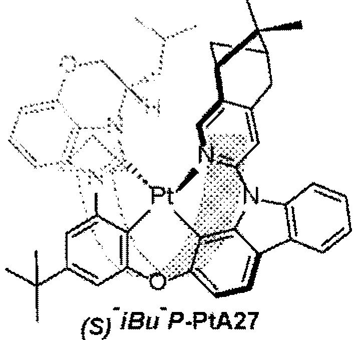

(1) Synthesis of (S)-iBu-LA27: following the synthesis procedure of (S)-iBu-LA6 in example 13, 15-Cl was used to replace 1-Cl to obtain the product as a yellow solid 420 mg in 70% yield. ¹H NMR (500 MHz, DMSO-d₆): δ(ppm) 0.66 (s, 3H), 0.96 (d, J=6.5 Hz, 3H), 1.00 (d, J=6.5 Hz, 3H), 1.19 (d, J=9.5 Hz, 1H), 1.36 (s, 9H), 1.42 (s, 3H), 1.71-1.77 (m, 1H), 1.79-1.87 (m, 1H), 1.93-1.99 (m, 1H), 2.27-2.32 (m, 1H), 2.71-2.76 (m, 1H), 2.93 (t, J=5.5 Hz, 1H), 3.03-3.12 (m, 2H), 4.48 (dd, J=12.0, 5.5 Hz, 1H), 4.67 (dd, J=13.0, 3.0 Hz, 1H), 4.89-4.93 (m, 1H), 7.16-7.21 (m, 3H), 7.33 (t, J=7.0 Hz, 1H), 7.36 (d, J=8.0 Hz, 1H), 7.43-7.47 (m, 2H), 7.52-7.57 (m, 3H), 7.59 (d, J=2.5 Hz, 1H), 7.76 (d, J=8.5 Hz, 1H), 8.21 (s, 1H), 8.23 (d, J=7.5 Hz, 1H), 8.31 (d, J=8.0 Hz, 1H), 10.18 (s, 1H).
(2) Synthesis of (S)-iBu-P—PtA27: following the synthesis procedure of (S)-iBu-P—PtA6 in example 13, (S)-iBu-LA27 was used to replace (S)-iBu-LA6 to obtain the product as a yellow solid 120 mg in 75% yield. ¹H NMR (500 MHz, DMSO-d₆): δ(ppm) 0.45 (d, J=7.0 Hz, 3H), 0.49 (s, 3H), 0.61 (d, J=7.0 Hz, 3H), 1.28 (d, J=9.5 Hz, 1H), 1.34 (s, 3H), 1.44 (s, 9H), 1.48-1.54 (m, 1H), 1.61-1.67 (m, 1H), 1.86-1.91 (m, 1H), 2.28-2.32 (m, 1H), 2.80-2.84 (m, 1H), 2.94 (d, J=17.5 Hz, 1H), 3.13-3.19 (m, 2H), 4.67-4.70 (m, 1H), 4.74-4.77 (m, H), 4.89-4.92 (m, 1H), 6.94 (d, J=8.0 Hz, 1H), 6.99 (d, J=1.5 Hz, 1H), 7.26 (d, J=8.0 Hz, 1H), 7.37-7.41 (m, 2H), 7.47-7.50 (m, 1H), 7.51 (d, J=1.5 Hz, 1H), 7.74 (d, J=8.5 Hz, 1H), 7.89 (d, J=8.0 Hz, 1H), 7.96 (d, J=8.0 Hz, 1H), 8.04 (s, 1H), 8.15 (dd, J=7.5, 2.0 Hz, 1H), 9.34 (s, 1H).

### Example 65

The synthetic route for (S′)-iPr—P—PtA28 is as follows:

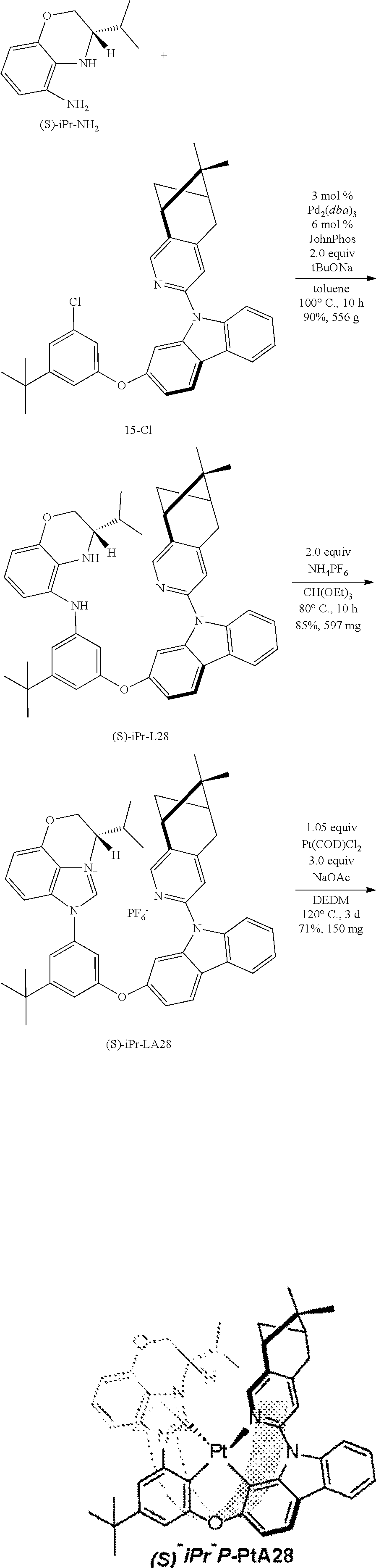

(1) Synthesis of (S)-iP-LA28: following the synthesis procedure of (S)-iPr-LA4 in example 10, 15-Cl was used to replace 1-Cl to obtain the product as a yellow solid 597 mg in 85% yield. ¹H NMR (500 MHz, DMSO-d₆): δ(ppm) 0.66 (s, 3H), 1.02-1.05 (m, 6H), 1.19 (d, J=9.5 Hz, 1H), 1.36 (s, 9H), 1.41 (s, 3H), 2.29-2.31 (m, 1H), 2.35-2.40 (m, 1H), 2.71-2.75 (m, 1H), 2.93 (t, J=5.5 Hz, 1H), 3.04-3.13 (m, 2H), 4.50 (dd, J=12.0, 3.0 Hz, 1H), 4.72-4.75 (m, 1H), 4.78 (dd, J=12.0, 4.0 Hz, 1H), 7.12-7.21 (m, 2H), 7.21 (t, J=2.0 Hz, 1H), 7.31-7.34 (m, 1H), 7.36 (d, J=8.5 Hz, 1H), 7.44-7.48 (m, 2H), 7.53 (t, J=8.5 Hz, 1H), 7.52-7.57 (m, 1H), 7.60 (d, J=2.0 Hz, 1H), 7.76 (d, J=8.0 Hz, 1H), 8.21 (s, 1H), 8.22-8.24 (m, 1H), 8.31 (d, J=8.5 Hz, 1H), 10.20 (s, 1H).
(2) Synthesis of (S)-iPr—P—PtA28: following the synthesis procedure of (S)-iPr—P—PtA4 in example 10, (S)-iP-LA28 was used to replace (S)-iPr-LA4 to obtain the product as a yellow solid 150 mg in 71% yield. ¹H NMR (500 MHz, DMSO-d₆): δ(ppm) 0.66 (s, 3H), 1.02-1.05 (m, 6H), 1.19 (d, J=9.5 Hz, 1H), 1.36 (s, 9H), 1.41 (s, 3H), 2.29-2.31 (m, 1H), 2.35-2.40 (m, 1H), 2.71-2.75 (m, 1H), 2.93 (t, J=5.5 Hz, 1H), 3.04-3.13 (m, 2H), 4.50 (dd, J=12.0, 3.0 Hz, 1H), 4.72-4.75 (m, 1H), 4.78 (dd, J=12.0, 4.0 Hz, 1H), 7.12-7.21 (m, 2H), 7.21 (t, J=2.0 Hz, 1H), 7.31-7.34 (m, 1H), 7.36 (d, J=8.5 Hz, 1H), 7.44-7.48 (m, 2H), 7.53 (t, J=8.5 Hz, 1H), 7.52-7.57 (m, 1H), 7.60 (d, J=2.0 Hz, 1H), 7.76 (d, J=8.0 Hz, 1H), 8.21 (s, 1H), 8.22-8.24 (m, 1H), 8.31 (d, J=8.5 Hz, 1H), 10.20 (s, 1H).

### Example 66

The synthetic route for (S,R)-Tjy-P—PtA29 is as follows:

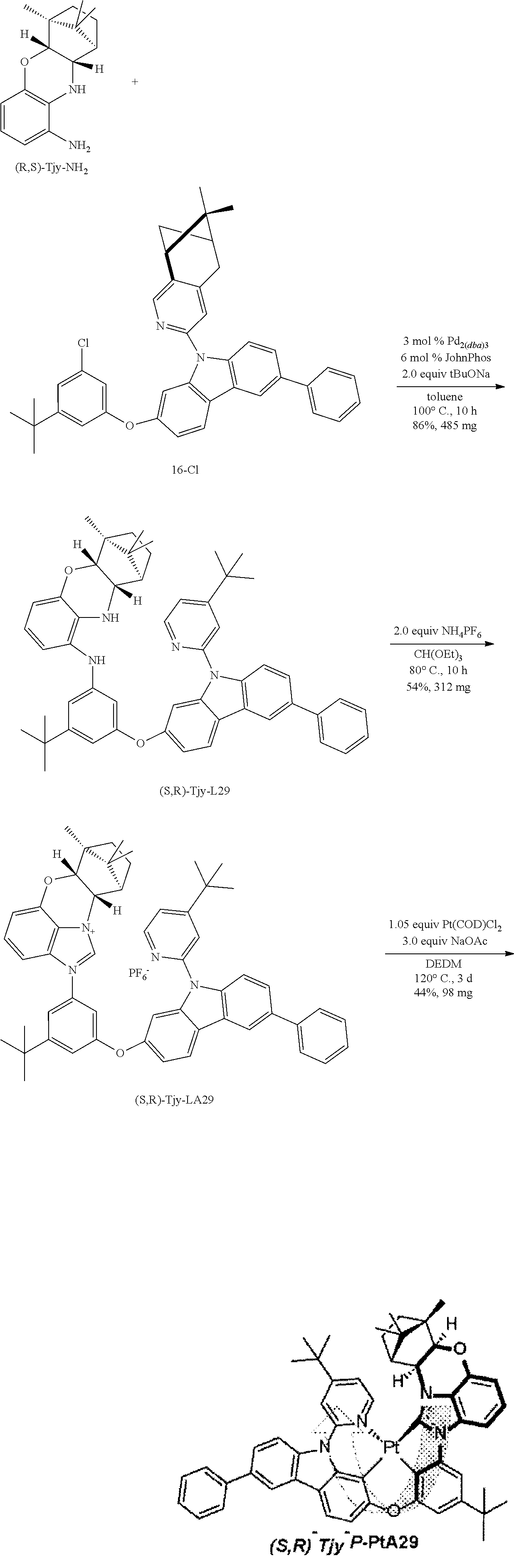

(1) Synthesis of (S,R)-Tjy-LA29: following the synthesis procedure of (S,R)-Tjy-LA25 in example 61, 16-Cl was used to replace 1-Cl to obtain the product as a yellow solid 312 mg in 54% yield. ¹H NMR (500 MHz, CDCl₃): δ(ppm) 0.52 (s, 3H), 0.74 (s, 3H), 0.90 (s, 3H), 1.19 (s, 3H), 1.30-1.35 (m, 2H), 1.39 (s, 9H), 1.46 (s, 3H), 1.65-1.78 (m, 2H), 1.98-2.03 (m, 1H), 2.35-2.40 (m, 1H), 2.58 (d, J=4.5 Hz, 1H), 2.75-2.80 (m, 1H), 2.94 (t, J=5.5 Hz, 1H), 3.11 (d, J=2.5 Hz, 2H), 4.56 (d, J=7.5 Hz, 1H), 4.90 (d, J=7.0 Hz, 1H), 7.00-7.02 (m, 2H), 7.11 (dd, J=8.5, 2.0 Hz, 1H), 7.23 (d, J=8.5 Hz, 1H), 7.33-7.41 (m, 2H), 7.43 (s, 1H), 7.46-7.50 (m, 4H), 7.64 (d, J=2.0 Hz, 1H), 7.68 (dd, J=8.5, 2.0 Hz, 1H), 7.70-7.74 (m, 2H), 7.77 (d, J=8.5 Hz, 1H), 8.15-8.22 (m, 2H), 8.29 (d, J=1.5 Hz, 1H), 9.47 (s, 1H).
(2) Synthesis of (S,R)-Tjy-P—PtA29: following the synthesis procedure of (S,R)-Tjy-P—PtA25 in example 61, (S,R)-Tjy-LA29 was used to replace (S,R)-Tjy-LA25 to obtain the product as a yellow solid 98 mg in 44% yield. ¹H NMR (500 MHz, CDCl₃): δ(ppm) 0.36 (s, 3H), 0.50 (s, 3H), 0.57-0.64 (m, 1H), 0.70 (s, 3H), 0.84-0.91 (m, 1H), 1.11 (s, 1H), 1.15 (s, 3H), 1.23-1.30 (m, 1H), 1.37 (s, 3H), 1.47 (s, 9H), 1.58-1.62 (m, 1H), 2.35-2.38 (m, 1H), 2.81-2.87 (m, 3H), 2.92 (d, J=18.5 Hz, 1H), 3.11 (dd, J=18.0, 3.0 Hz, 1H), 4.49 (d, J=7.0 Hz, 1H), 4.72 (d, J=7.5 Hz, 1H), 6.78 (d, J=8.0 Hz, 1H), 7.18 (d, J=1.5 Hz, 1H), 7.30 (t, J=8.0 Hz, 1H), 7.36-7.40 (m, 1H), 7.45 (d, J=8.0 Hz, 1H), 7.48-7.56 (m, 4H), 7.64 (dd, J=8.5, 2.0 Hz, 1H), 7.74-7.78 (m, 2H), 7.88 (d, J=8.5 Hz, 1H), 7.95 (d, J=8.5 Hz, 1H), 8.09 (s, 1H), 8.25 (d, J=1.5 Hz, 1H), 9.26 (s, 1H).

### Example 67

The synthetic route for (R,S)-Acr-M-PtF4 is as follows:

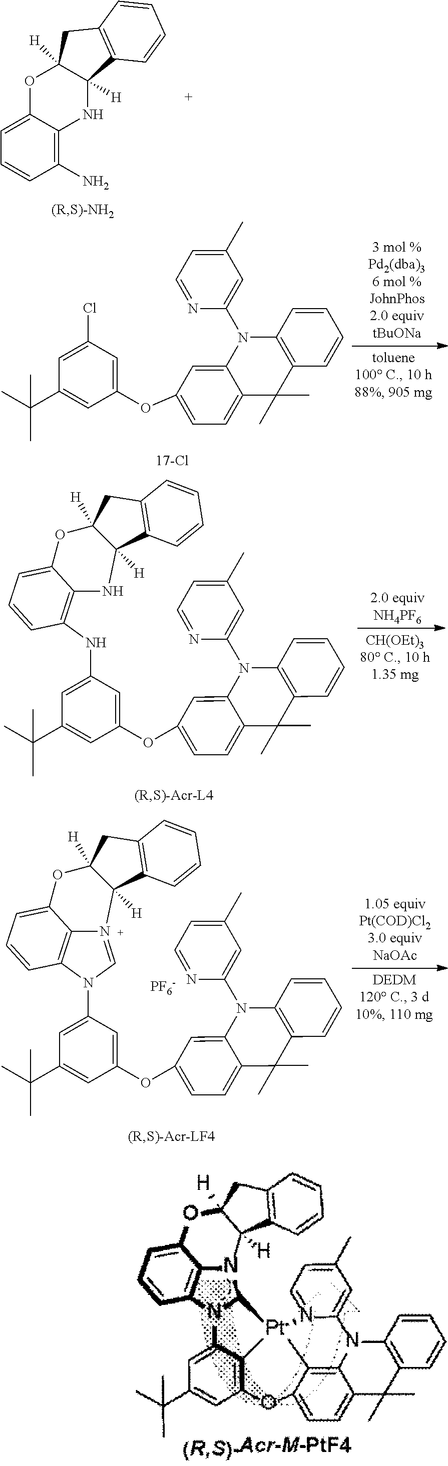

(1) Synthesis of (R,S)-Acr-L4: following the synthesis procedure of (R,S)-LA1 in example 1, 17-Cl was used to replace 1-Cl to obtain the product as a yellow solid 1.35 g.
(2) Synthesis of (R,S)-Acr-M-PtF4: following the synthesis procedure of (R,S)-M-PtA1 in example 1, (R,S)-Acr-L4 was used to replace (R,S)-LA1 to obtain the product as a yellow solid 140 mg in 10% yield. ¹H NMR (500 MHz, DMSO-d₆): δ(ppm) 1.44 (s, 9H), 1.88 (s, 3H), 2.14 (s, 4H), 3.33-3.38 (m, 1H), 3.45 (dd, J=16.5, 3.5 Hz, 1H), 5.44 (t, J=4.0 Hz, 1H), 6.33 (d, J=3.5 Hz, 1H), 6.65 (dd, J=8.0, 1.5 Hz, 1H), 6.85 (d, J=8.0 Hz, 1H), 6.91 (dd, J=6.5, 2.0 Hz, 1H), 6.97-7.00 (m, 3H), 7.05-7.08 (m, 2H), 7.18-7.22 (m, 2H), 7.31 (t, J=7.5 Hz, 1H), 7.35 (t, J=8.5 Hz, 1H), 7.42 (d, J=7.0 Hz, 1H), 7.51-7.54 (m, 2H), 7.73 (d, J=8.5 Hz, 1H), 7.87 (d, J=8.0 Hz, 1H), 9.41 (d, J=6.0 Hz, 1H).

### Example 68

The synthetic route for (S,R)-Acr-P—PtF4 is as follows:

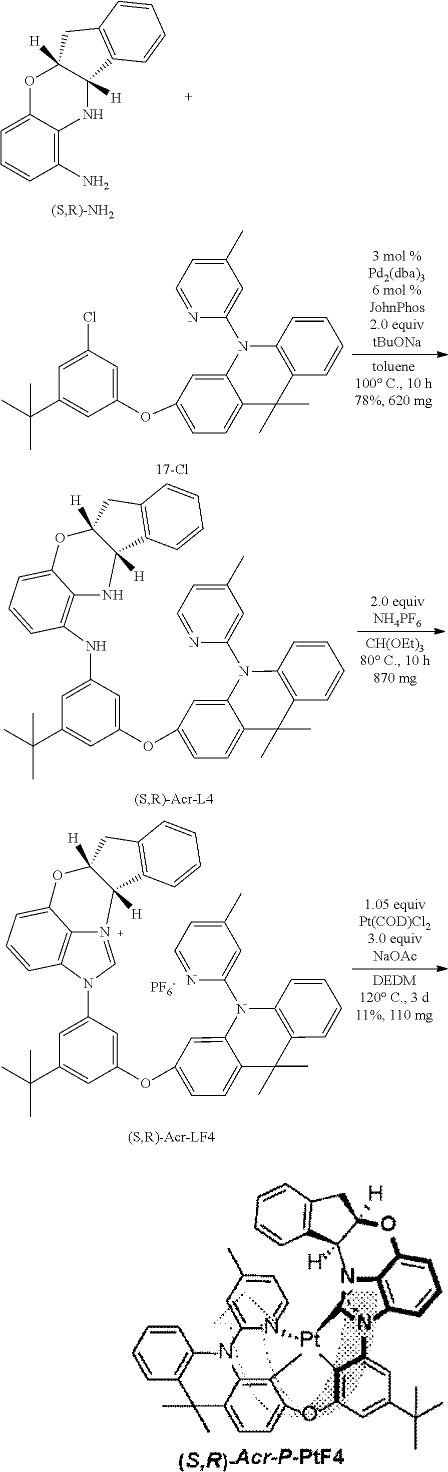

(1) Synthesis of (S,R)-Acr-L4: following the synthesis procedure of (S,R)-LA1 in example 2, 17-Cl was used to replace 1-Cl to obtain the product as a yellow solid 870 mg.
(2) Synthesis of (S,R)-Acr-P—PtF4: following the synthesis procedure of (S,R)—P—PtA1 in example 2, (S,R)-Acr-L4 was used to replace (S,R)-LA1 to obtain the product as a yellow solid 110 mg in 11% yield. ¹H NMR (500 MHz, DMSO-d₆): δ(ppm) 1.24 (s, 3H), 1.43 (s, 9H), 1.88 (s, 3H), 2.13 (s, 3H), 3.33-3.38 (m, 1H), 3.34-3.46 (m, 1H), 5.44 (t, J=4.0 Hz, 1H), 6.32 (d, J=3.5 Hz, 1H), 6.64 (dd, J=8.5, 2.0 Hz, 1H), 6.85 (d, J=8.0 Hz, 1H), 6.91 (dd, J=6.0, 1.5 Hz, 1H), 6.96-6.99 (m, 3H), 7.05-7.08 (m, 2H), 7.18-7.21 (m, 2H), 7.31 (t, J=7.0 Hz, 1H), 7.35 (t, J=8.0 Hz, 1H), 7.41 (d, J=7.5 Hz, 1H), 7.51-7.54 (m, 2H), 7.73 (d, J=8.5 Hz, 1H), 7.87 (d, J=7.5 Hz, 1H), 9.41 (d, J=6.0 Hz, 1H).

### Example 69

The synthetic route for (S)—CMe-P—Pt30 is as follows:

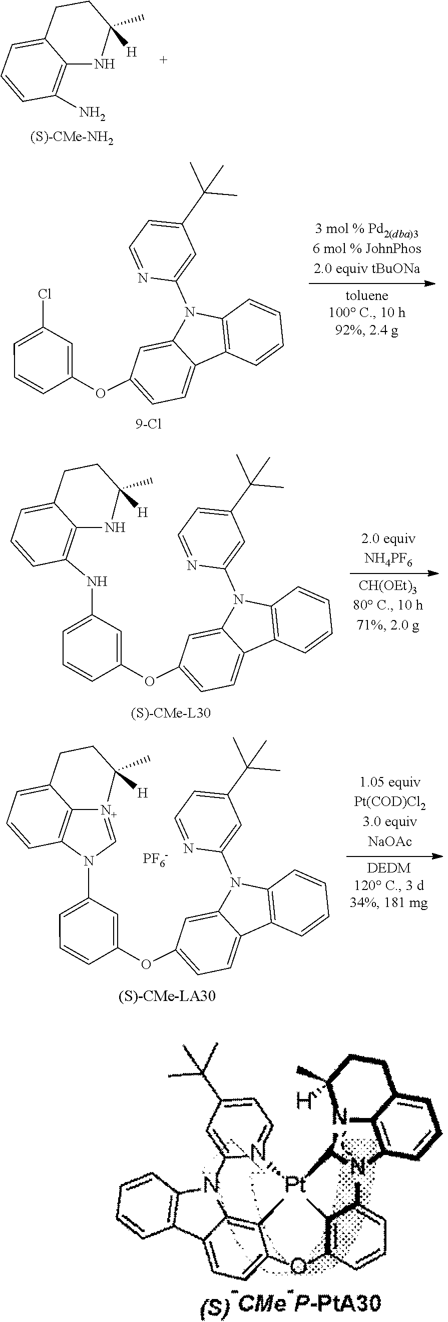

(1) Synthesis of (S)—CMe-LA30: following the synthesis procedure of (S)-iPr-LA20 in example 51, (S)—CMe-NH₂ was used to replace (S)-iPr-NH₂ to obtain the product as a yellow solid 2.0 g in 71% yield.
(2) Synthesis of (S)—CMe-P—PtA30: following the synthesis procedure of (S)-iPr—P—PtA20 in example 51, (S)—CMe-LA3 was used to replace (S)-iPr-LA20 to obtain the product as a yellow solid 181 g in 34% yield. ¹H NMR (500 MHz, DMSO-d₆): δ(ppm) 1.29 (s, 12H), 2.16 (m, 1H), 2.41 (s, 1H), 3.01 (s, 2H), 5.07 (s, 1H), 6.96 (dd, J=8.0, 1.0 Hz, 1H), 7.25 (m, 4H), 7.40 (td, J=7.5, 3.0 Hz, 2H), 7.48 (m, 1H), 7.57 (d, J=8.0 Hz, 1H), 7.91 (d, J=8.0 Hz, 1H), 8.01-8.11 (m, 3H), 8.16 (d, J=7.5 Hz, 1H), 9.60 (d, J=6.0 Hz, 1H).

### Example 70

The synthetic route for (S)-iPr—P—Pt31 is as follows:

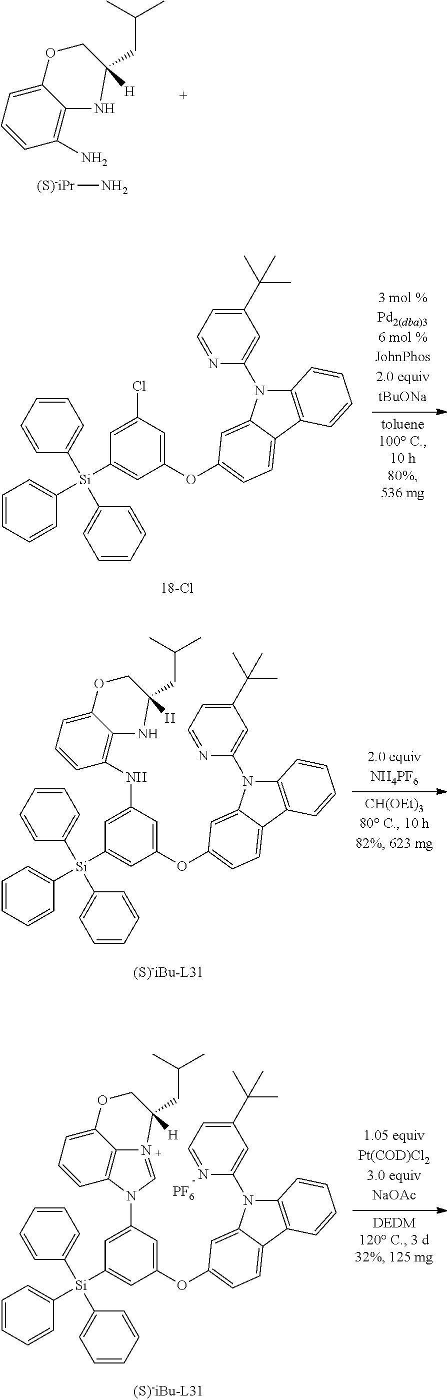

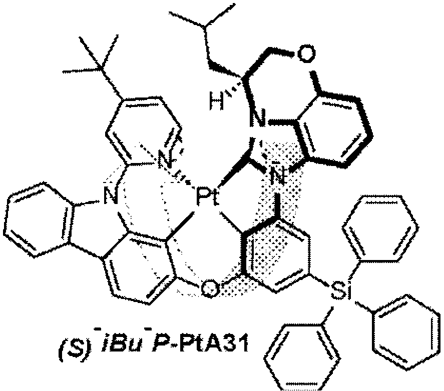

(1) Synthesis of (S)-iBu-LA31: following the synthesis procedure of (S)-iBu-LA6 in example 13, 18-Cl was used to replace 1-Cl to obtain the product as a yellow solid 623 mg in 82% yield. MS: m/z 886.4 (M+H)+.
(2) Synthesis of (S)-iBu-P—PtA31: following the synthesis procedure of (S)-iBu-P—PtA6 in example 13, (S)-iBu-LA31 was used to replace (S)-iBu-LA6 to obtain the product as a yellow solid 125 mg in 35% yield. MS: m/z 1058.3 (M+H)+.

### Example 71

The synthetic route for (S)—Cring-iBu-P—PtA32 is as follows:

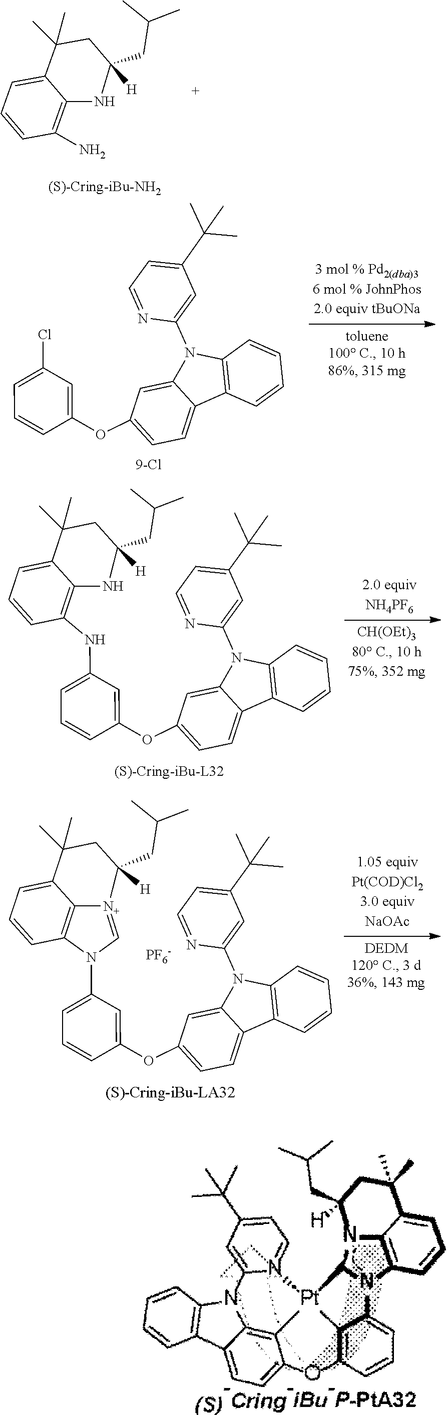

(1) Synthesis of (S)—Cring-iP-LA32: following the synthesis procedure of (S)-iBu-LA6 in example 13, (S)—Cring-iPr-NH₂ was used to replace (S)-iBu-NH₂ to obtain the product as a yellow solid 352 mg in 75% yield. MS: m/z 606.35 (M+H)⁺.
(2) Synthesis of (S)—Cring-iP—P—PtA32: following the synthesis procedure of (S)-iBu-P—PtA6 in example 13, (S)—Cring-iP-LA32 was used to replace (S)-iBu-LA6 to obtain the product as a yellow solid 143 mg in 36% yield. MS: m/z 826.38 (M+H)⁺.

### Example 72

The synthetic route for (R)-2MeiPr-M-PtA5 is as follows:

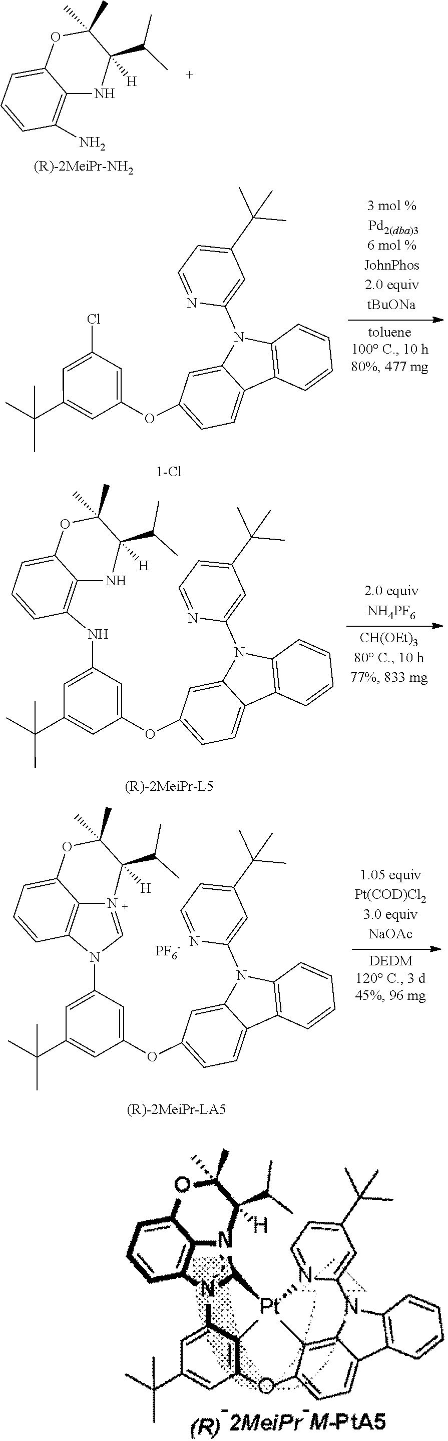

(1) Synthesis of (R)-2MeiPr-LA5: following the synthesis procedure of (S)-2MeiPr-LA5 in example 11, (R)-2MeiPr-NH₂ was used to replace (S)-2MeiPr-NH₂ to obtain the product as a yellow solid 833 mg in 77% yield. ¹H NMR (500 MHz, DMSO-d₆): δ(ppm) 0.54 (d, J=7.0 Hz, 3H), 1.08 (d, J=7.0 Hz, 3H), 1.23 (s, 3H), 1.29 (s, 9H), 1.37 (s, 9H), 1.70 (s, 3H), 2.54-2.59 (m, 1H), 4.70 (d, J=2.5 Hz, 1H), 7.11 (d, J=8.0 Hz, 1H), 7.21 (dd, J=8.5, 2.0 Hz, 1H), 7.29 (d, J=8.5 Hz, 1H), 7.32-7.36 (m, 1H), 7.39 (t, J=2.0 Hz, 1H), 7.44-7.49 (m, 3H), 7.49-7.54 (m, 2H), 7.66 (t, J=2.0 Hz, 1H), 7.68 (d, J=1.0 Hz, 1H), 7.74 (d, J=8.0 Hz, 1H), 8.24 (d, J=7.5 Hz, 1H), 8.32 (d, J=8.5 Hz, 1H), 8.58 (d, J=5.5 Hz, 1H), 10.19 (s, 1H).
(2) Synthesis of (R)-2MeiPr-M-PtA5: following the synthesis procedure of (S)-2MeiPr—P—PtA5 in example 11, (R)-2MeiPr-LA5 was used to replace (S)-2MeiPr-LA5 to obtain the product as a yellow solid 96 mg in 45% yield. ¹H NMR (500 MHz, DMSO-d₆): δ(ppm) 0.30 (d, J=7.0 Hz, 3H), 0.96 (d, J=7.0 Hz, 3H), 1.37 (s, 9H), 1.45 (s, 9H), 1.58 (s, 3H), 1.67 (s, 3H), 3.37-3.38 (m, 1H), 4.51 (d, J=2.5 Hz, 1H), 6.90 (d, J=8.0 Hz, 1H), 7.00 (d, J=1.0 Hz, 1H), 7.28 (d, J=8.5 Hz, 1H), 7.39-7.43 (m, 2H), 7.46 (dd, J=6.0, 2.0 Hz, 1H), 7.47-7.52 (m, 1H), 7.57 (d, J=1.5 Hz, 1H), 7.76 (d, J=8.5 Hz, 1H), 7.90 (d, J=8.0 Hz, 1H), 8.07 (d, J=8.0 Hz, 1H), 8.13-8.19 (m, 2H), 9.39 (d, J=6.5 Hz, 1H).

### Example 73

The synthetic route for (S)-iPr—P—Pt33 is as follows:

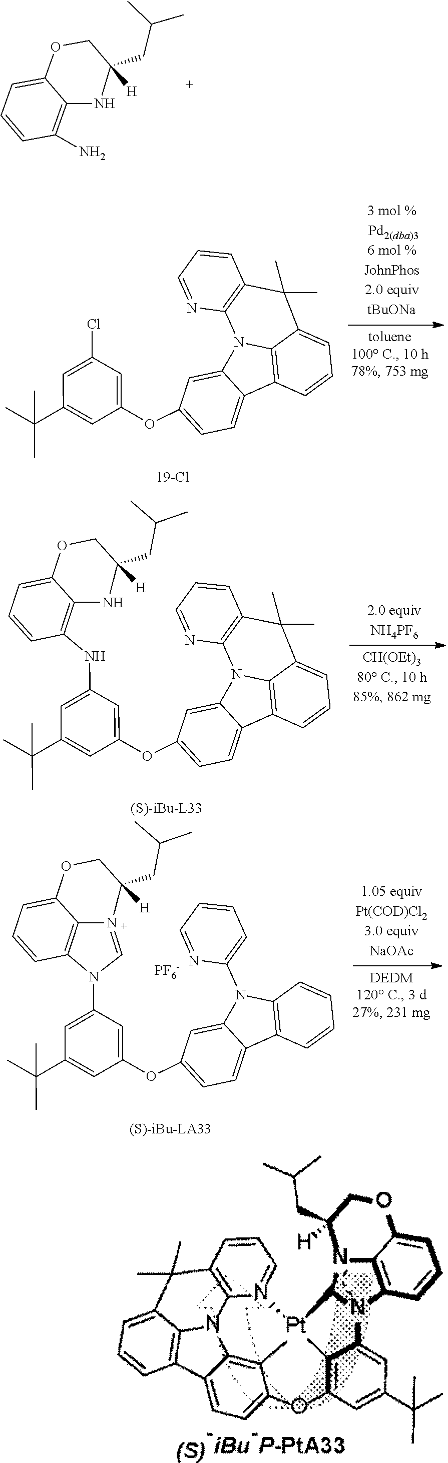

(1) Synthesis of (S)-iBu-LA33: following the synthesis procedure of (S)-iBu-LA6 in example 13, 19-Cl was used to replace 1-Cl to obtain the product as a yellow solid 862 mg in 85% yield. MS: m/z 648.3 (M+H)⁺.
(2) Synthesis of (S)-iBu-P—PtA33: following the synthesis procedure of (S)-iBu-P—PtA6 in example 13, (S)-iBu-LA33 was used to replace (S)-iBu-LA6 to obtain the product as a yellow solid 231 mg in 27% yield. MS: m/z 840.3 (M+H)⁺.

### Example 74

The synthetic route for (S)-iPr—P—Pt34 is as follows:

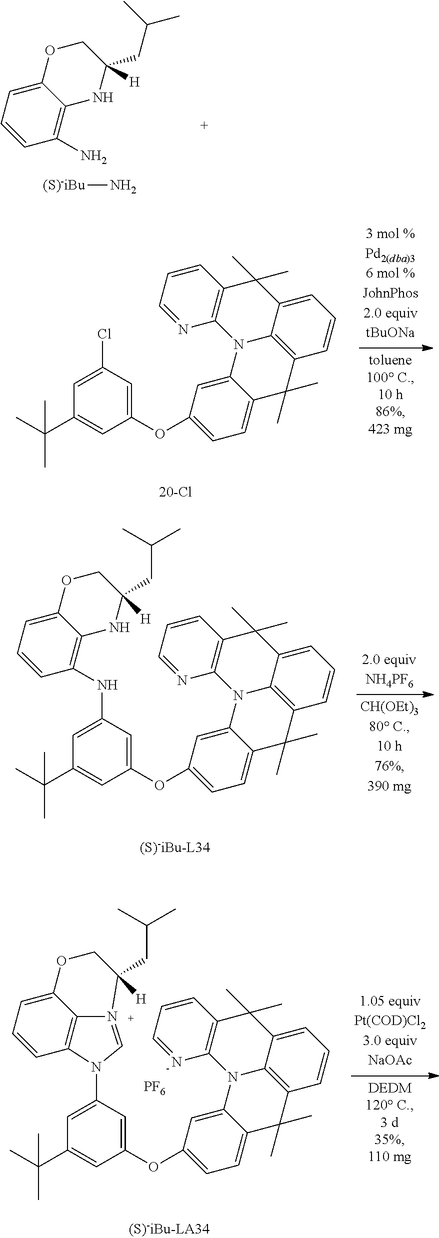

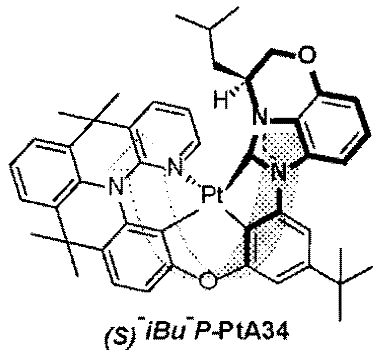

(1) Synthesis of (S)-iBu-LA34: following the synthesis procedure of (S)-iBu-LA6 in example 13, 20-Cl was used to replace 1-Cl to obtain the product as a yellow solid 390 mg in 76% yield. MS: m/z 690.4 (M+H)⁺.
(2) Synthesis of (S)-iBu-P—PtA34: following the synthesis procedure of (S)-iBu-P—PtA6 in example 13, (S)-iBu-LA34 was used to replace (S)-iBu-LA6 to obtain the product as a yellow solid 110 mg in 35% yield. MS: m/z 882.3 (M+H)⁺.

### Example 75

The synthetic route for (S)-iPr—P—Pt35 is as follows:

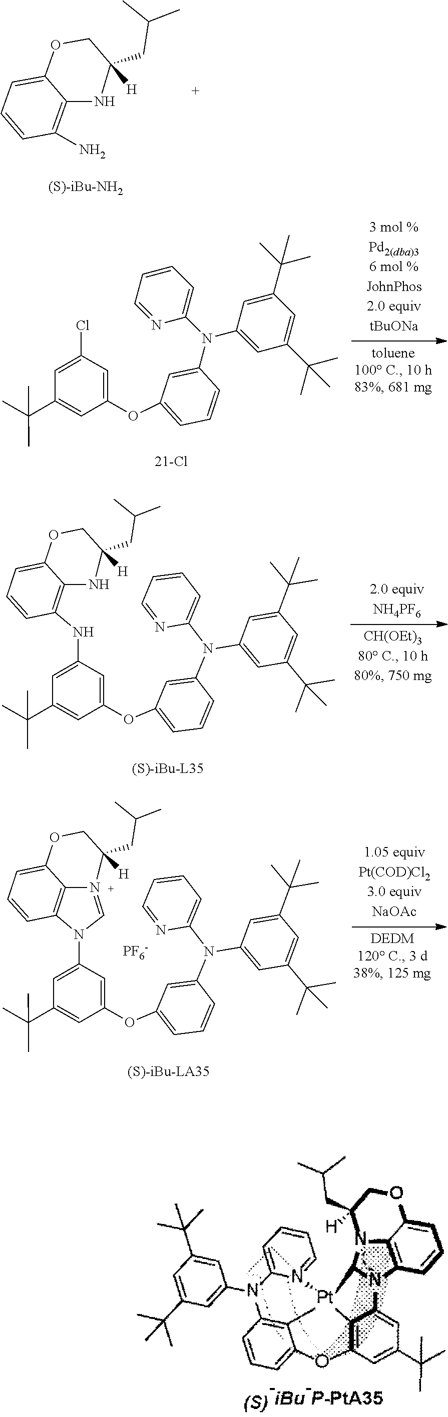

(1) Synthesis of (S)-iBu-LA35: following the synthesis procedure of (S)-iBu-LA6 in example 13, 21-Cl was used to replace 1-Cl to obtain the product as a yellow solid 750 mg in 80% yield. MS: m/z 722.5 (M+H)⁺.
(2) Synthesis of (S)-iBu-P—PtA35: following the synthesis procedure of (S)-iBu-P—PtA6 in example 13, (S)-iBu-LA35 was used to replace (S)-iBu-LA6 to obtain the product as a yellow solid 125 mg in 38% yield. MS: m/z 914.4 (M+H)⁺.

### Example 76

The synthetic route for (R,S)-M-Pt36 is as follows:

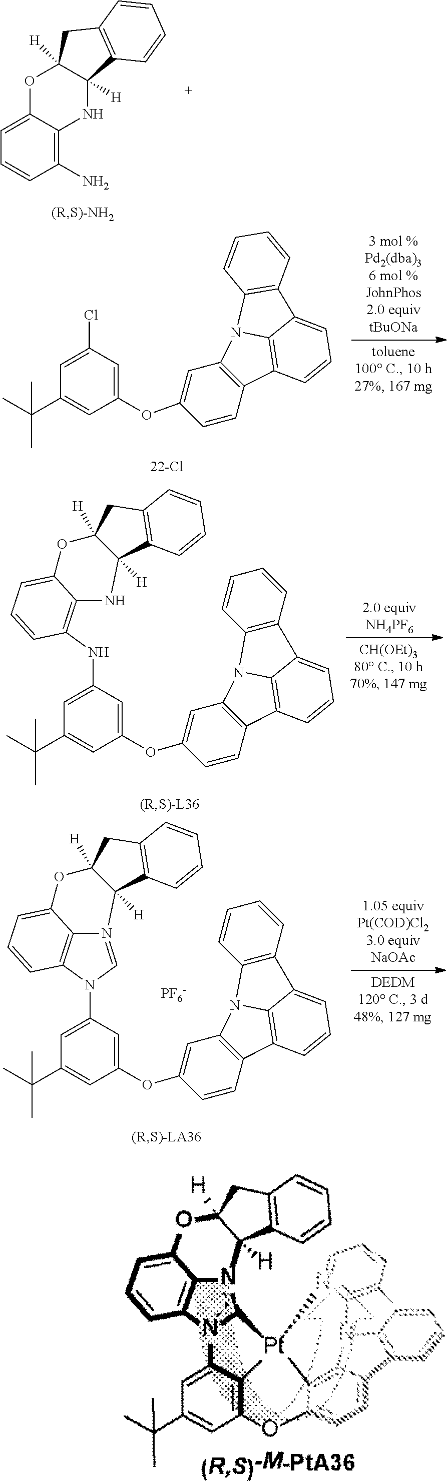

(1) Synthesis of (R,S)-LA36: following the synthesis procedure of (R,S)-LA1 in example 1, 22-Cl was used to replace 1-Cl to obtain the product as a yellow solid 147 mg in 70% yield. ¹H NMR (500 MHz, DMSO-d₆): δ(ppm) (s, 9H), 3.27-3.31 (m, 1H), 3.53 (dd, J=16.5, 5.0 Hz, 1H), 5.40-5.43 (m, 1H), 6.34 (d, J=4.0 Hz, 1H), 7.16 (dd, J=7.0, 1.5 Hz, 1H), 7.29-7.34 (m, 2H), 7.38 (t, J=7.5 Hz, 1H), 7.42-7.49 (m, 5H), 7.60-7.61 (m, 2H), 7.68-7.72 (m, 2H), 7.91 (d, J=2.5 Hz, 1H), 8.21 (d, J=7.5 Hz, 1H), 8.25 (d, J=7.5 Hz, 1H), 8.38 (d, J=8.5 Hz, 1H), 8.54 (dd, J=5.0, 1.5 Hz, 1H), 8.70 (dd, J=8.0, 2.0 Hz, 1H), 10.49 (s, 1H).
(2) Synthesis of (R,S)-M-PtA36: following the synthesis procedure of (R,S)-M-PtA1 in example 1, (R,S)-LA36 was used to replace (R,S)-LA1 to obtain the product as a yellow solid 127 mg in 48% yield. MS: m/z 830.2 (M+H)⁺.

### Example 77

The synthetic route for (S,R)—P—Pt36 is as follows:

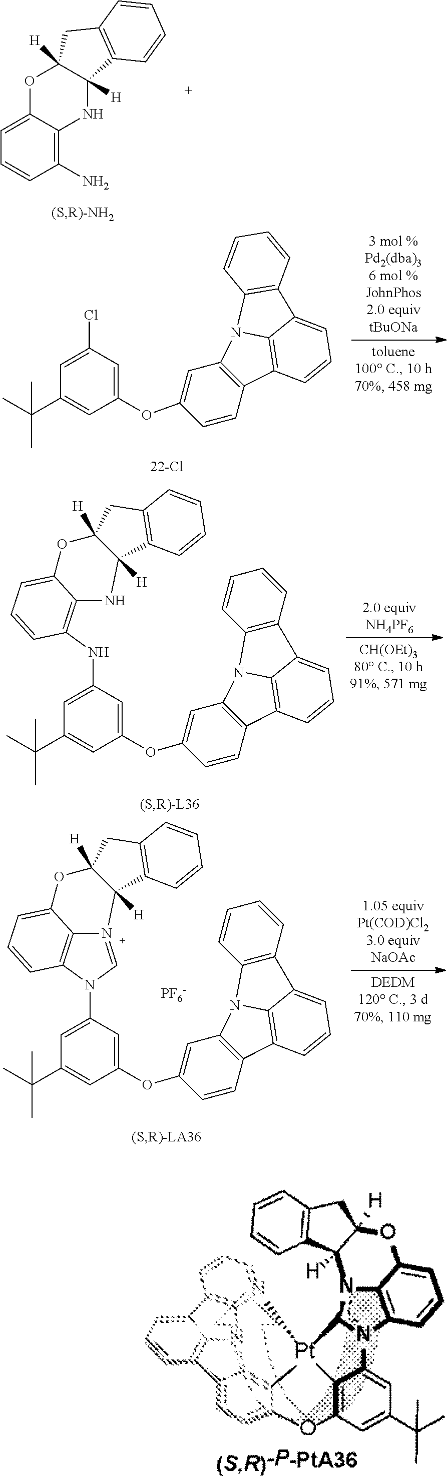

(1) Synthesis of (S,R)-LA36: following the synthesis procedure of (S,R)-LA1 in example 2, 22-Cl was used to replace 1-Cl to obtain the product as a yellow solid 571 mg in 91% yield. MS: m/z 637.3 (M+H)⁺.
(2) Synthesis of (S,R)—P—Pt36: following the synthesis procedure of (S,R)—P—PtA1 in example 1, (S,R)-LA36 was used to replace V to obtain the product as a yellow solid 110 mg in 70% yield. MS: m/z 830.3 (M+H)⁺.

### Example 78

The synthetic route for (S)-iPr—P—Pt37 is as follows:

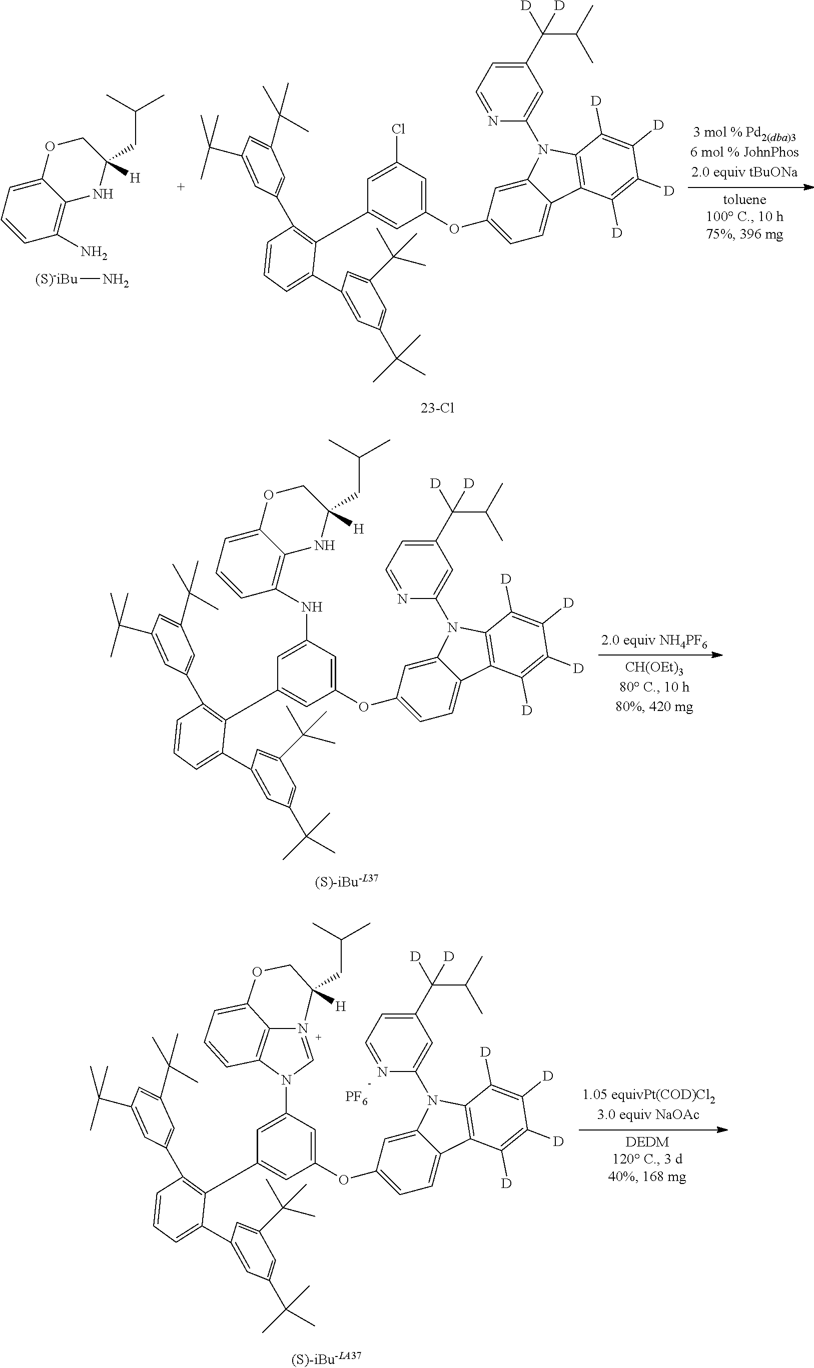

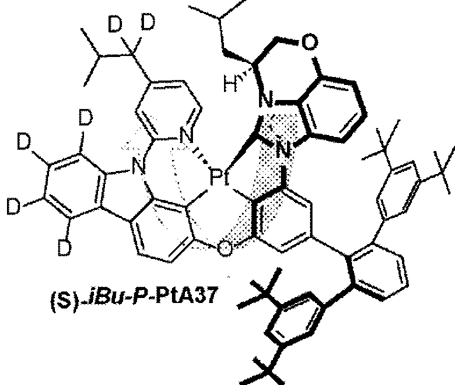

(1) Synthesis of (S)-iBu-LA37: following the synthesis procedure of (S)-iBu-LA6 in example 13, 23-Cl was used to replace 1-Cl to obtain the product as a yellow solid 420 mg in 80% yield. MS: m/z 1066.7 (M+H)⁺.
(2) Synthesis of (S)-iBu-P—PtA37: following the synthesis procedure of (S)-iBu-P—PtA6 in example 13, (S)-iBu-LA37 was used to replace (S)-iBu-LA6 to obtain the product as a yellow solid 168 mg in 40% yield. MS: m/z 1258.6 (M+H)⁺.

### Example 79

The synthetic route for (S)-iBuD-P—PtA38 is as follows:

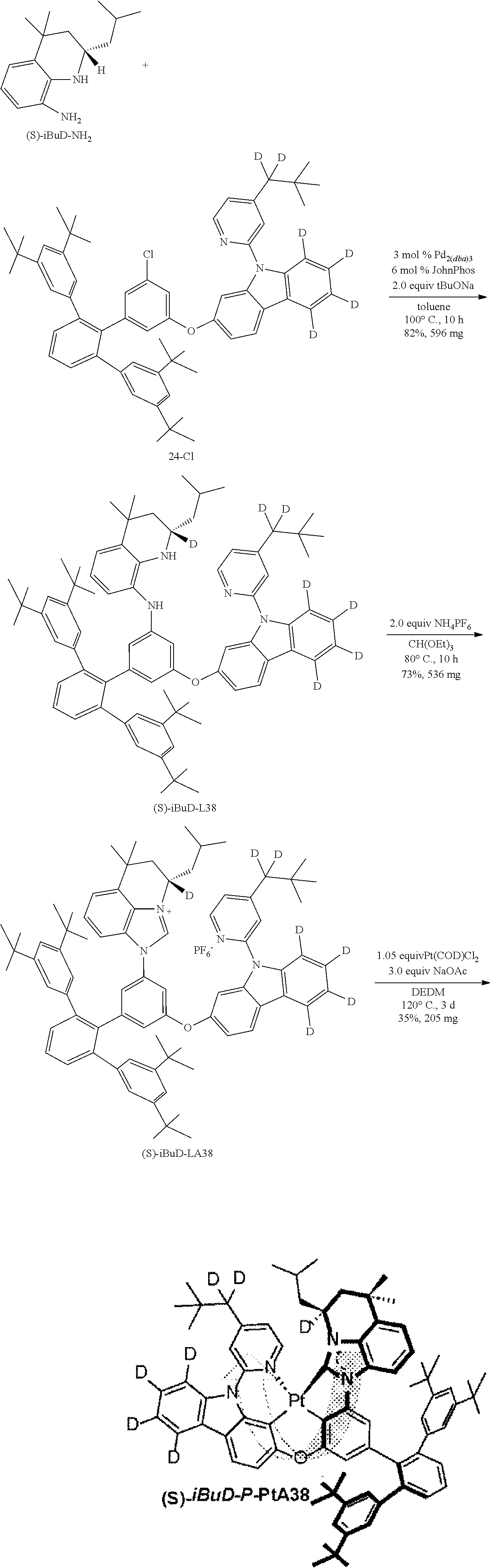

(1) Synthesis of (S)-iBu-LA38: following the synthesis procedure of (S)-iBu-LA6 in example 13, (S)-iBuD-NH₂ was used to replace (S)-iBu-NH₂ and substitute 1-Cl with 24-Cl to obtain the product as a yellow solid 536 mg in 73% yield. MS: m/z 1107.8 (M+H)⁺.
(2) Synthesis of (S)-iBuD-P—PtA38: following the synthesis procedure of (S)-iBu-P—PtA6 in example 13, (S)-iBuD-LA38 was used to replace (S)-iBuD-LA38 to obtain the product as a yellow solid 305 mg in 35% yield. MS: m/z 1299.7 (M+H)⁺.

### Example 80

The synthetic route for (S)-2MeiBu-P—PtA39 is as follows:

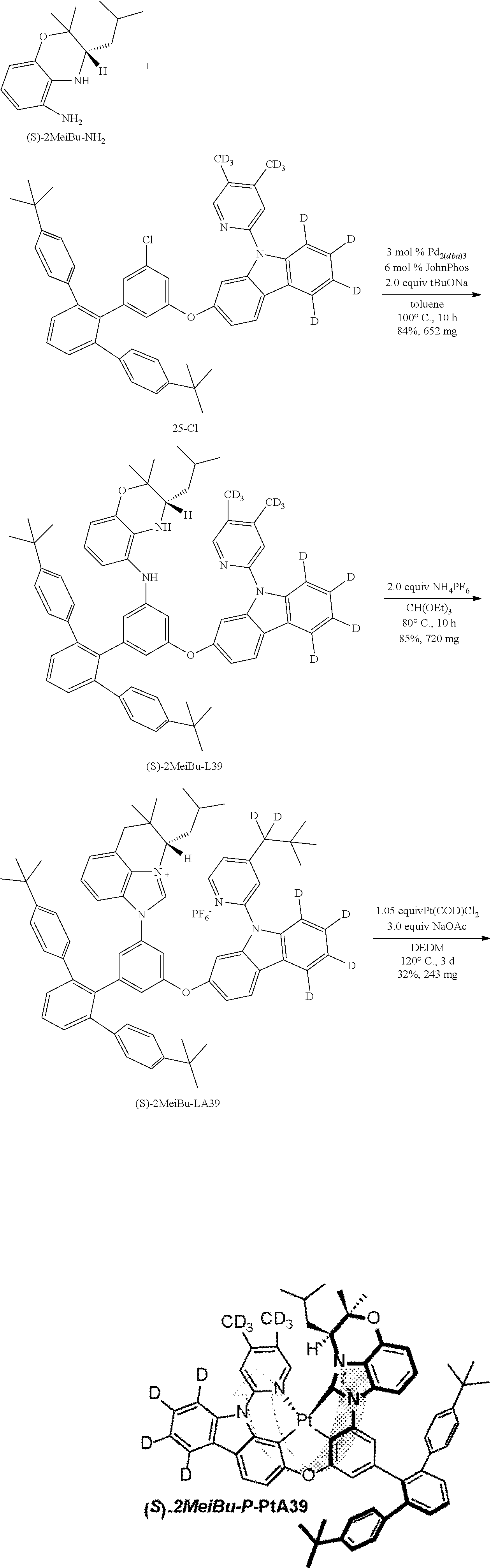

(1) Synthesis of (S)-2MeiBu-LA39: following the synthesis procedure of (S)-2MeiBu-LA7 in example 15, (S)-2MeiBu-NH₂ was used to replace (S)-2MeiBu-NH₂ and substitute 1-Cl with 25-Cl to obtain the product as a yellow solid 720 mg in 85% yield. MS: m/z 958.6 (M+H)⁺.
(2) Synthesis of (S)-2MeiBu-P—PtA7: following the synthesis procedure of (S)-2MeiBu-P—PtA7 in example 15, (S)-2MeiBu-LA39 was used to replace (S)-2MeiBu-LA7 to obtain the product as a yellow solid 243 mg in 32% yield. MS: m/z 1150.5 (M+H)⁺.

### Example 81

The synthetic route for (S)-2MeiBu-P—PtA40 is as follows:

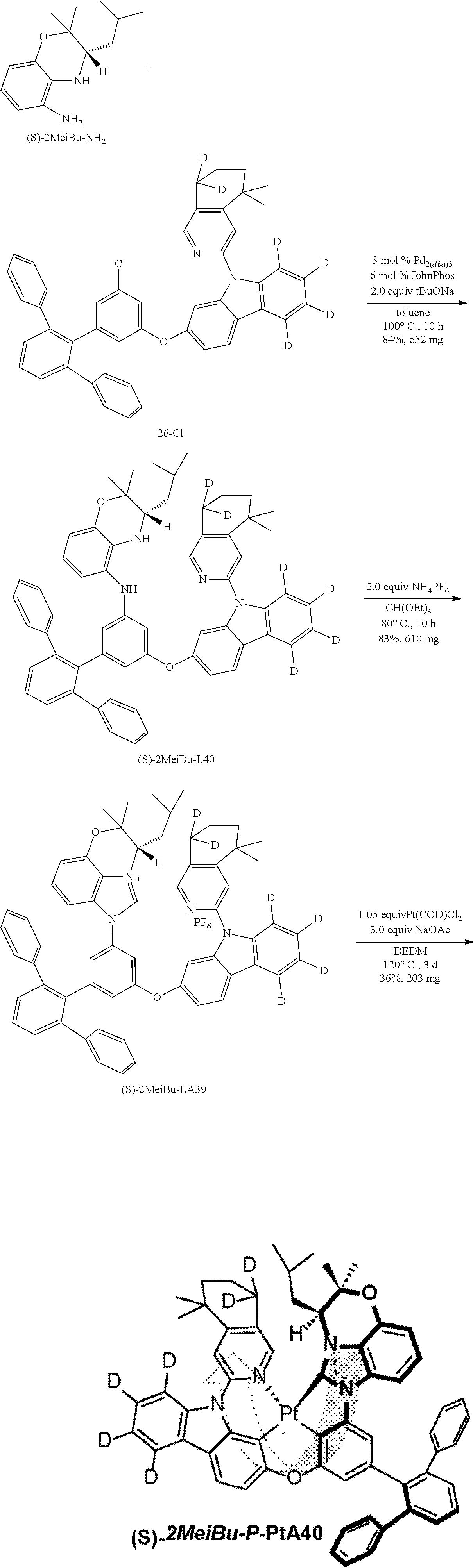

(1) Synthesis of (S)-2MeiBu-LA40: following the synthesis procedure of (S)-2MeiBu-LA7 in example 15, 26-Cl was used to replace 1-Cl to obtain the product as a yellow solid 610 mg in 83% yield. MS: m/z 896.5 (M+H)⁺.
(2) Synthesis of (S)-2MeiBu-P—PtA40: following the synthesis procedure of (S)-2MeiBu-P—PtA7 in example 15, (S)-2MeiBu-LA40 was used to replace (S)-2MeiBu-LA7 to obtain the product as a yellow solid 203 mg in 36% yield. MS: m/z 1088.5 (M+H)⁺.

### Electrochemical, Photophysical Measurements and Theoretical Calculations:

Absorption spectra was recorded on an Agilent 8453 UV-Vis spectrometer. Steady-state emission experiments and lifetime measurements were performed on using a Horiba Jobin Yvon FluoroLog-3 spectrometer. Alternatively, steady-state emission spectra were measured using a HITACHI F-7000 spectrometer. Low-temperature (77 K) emission spectra and lifetimes were measured in 2-methyltetrahydrofuran (2-MeTHF) solution, which was cooled with liquid nitrogen. The Pt(II) complex was analyzed theoretically using the Gaussian 09 software package. The geometry of the ground state (S₀) molecule was optimized using the density functional theory (DFT) method. DFT calculations employed the B3LYP functional with the 6-31G(d) basis set for C, H, O, and N atoms, and the LANL2DZ basis set for Pt and Pd atoms. Enantiomeric purity (ee value) was assessed using a chiral column EnantiopakR-C (specifications: 4.6×250 mm, 5 m).

### Experimental Data and Analyses:

FIG. 3 shows the design concept of circularly polarized luminescent materials for optically pure helically chiral tetradentate cyclometalated complexes centered on metal ions Pt(II) or Pd(II). The optically pure raw materials are economically and easily obtainable; central chirality autonomously induces the generation of helical chirality; the optically pure circularly polarized luminescent materials unneed chiral separation, greatly saving the preparation cost of optical pure materials, and can be prepared in large quantities without being limited by chiral preparation of chromatographic column separation.

From the above embodiments 1 to 33, it can be seen that the formation of helically chiral metal complex molecules can be induced by the central chiral fragment in the ligand to autonomously coordinate the entire tetradentate ligand with metal ions in a low steric hindrance manner, forming optically pure helically chiral metal complex circularly polarized luminescent materials. This induced reaction has stereospecificity. As shown in Example 1, ligand (R,S)-LA1 stereoselectively induces the formation of M-shaped helically chiral metal complexes (R,S)-M-PtA1; while in Example 2, ligand (S,R)-LA1 stereospecifically induces the formation of P-shaped spiral chiral metal complexes (S,R)—P—PtA1, etc. In addition, spiral chiral metal complexes can be prepared in large quantities from corresponding chiral raw materials without the need for chiral separation.

From the distribution of electrons and holes in the lowest excited singlet state (T₁) of (S,R)—P—PtA1 and its enantiomer (R,S)-M-PtA1 in FIG. 4, it can be seen that the benzocarbene portion makes a significant contribution to the T₁ state of the material molecule.

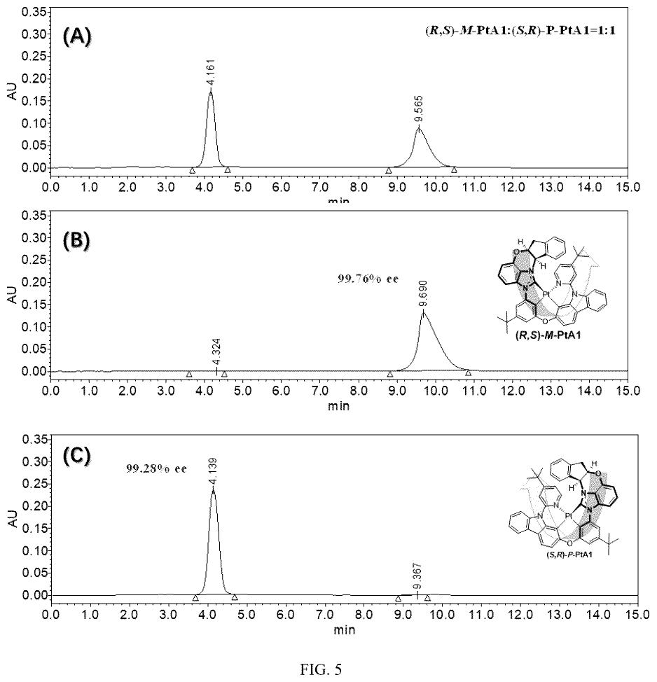

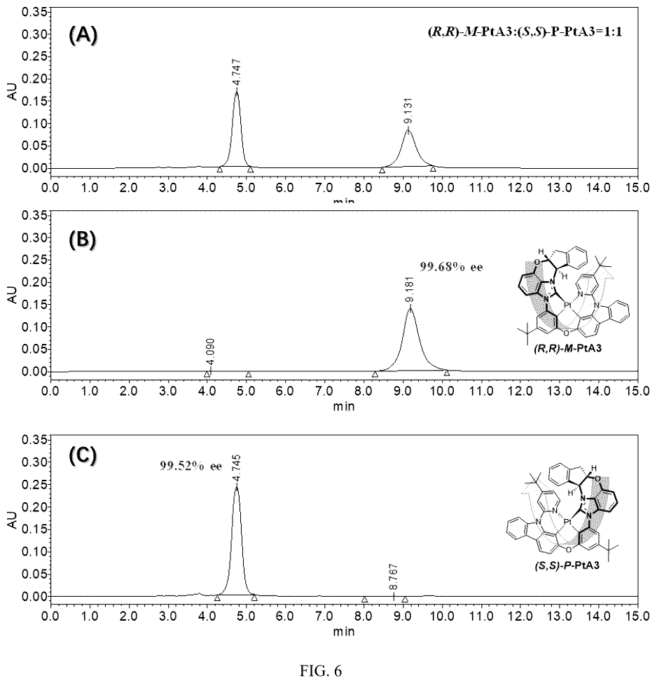

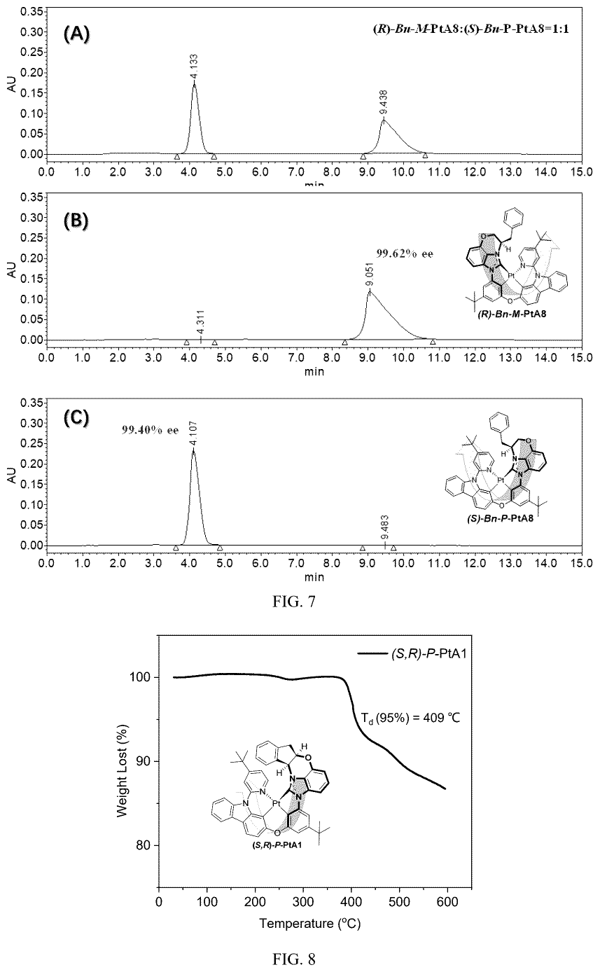

According to the excess values of enantiomers (ee values) in Table 1, it can be seen that the single chiral material molecules obtained from the above material design concept are all of extremely high optical purity, with ee values >99%; the results of high-performance liquid chromatography analysis of some materials in FIGS. 5 to 7 also confirm that the helically chiral metal complexes synthesized by the above method have high optical purity, with ee values >99%. The above experimental data also indicates that the design concept of these materials are successful.

From the specific rotation data of some chiral ligands and chiral metal complexes in Table 1, it can be seen that even ligands with the same central chirality and chiral metal complexes have a huge difference in specific rotation values, and even the rotation direction is completely opposite, indicating that the helical chirality center in the metal complex has a decisive impact on the optical rotation properties of the entire compound. Due to the fact that excited radiative luminescence of metal complexes mainly involves metal to ligand charge transfer states (MLCT) and intra ligand charge transfer states (ILCT), the helically chiral center of metal complexes will have a significant impact on their circularly polarized optical properties. In contrast, the comparison complex PtON1-tt and PdON1-tt without central chirality have no optical rotation and zero optical rotation ([a]²⁰_{D}), indicating that they are not chiral molecules.

The thermogravimetric analysis curve of (S,R)—P—PtA1 in FIG. 8 shows that its decomposition temperature (temperature at 5% mass loss) is as high as 409° C.; this type of material has high thermal stability. Moreover, the sublimation experiment showed that the (S,R)—P—PtA1 material sublimated at 320-340° C. did not undergo racemization and had an ee value of >99% after manual HPLC analysis. The above experiments indicate that molecules of this type of material have high thermal and chemical stability, and their optical purity will not be reduced due to sublimation purification of the material, thereby weakening or losing its circularly polarized luminescent properties.

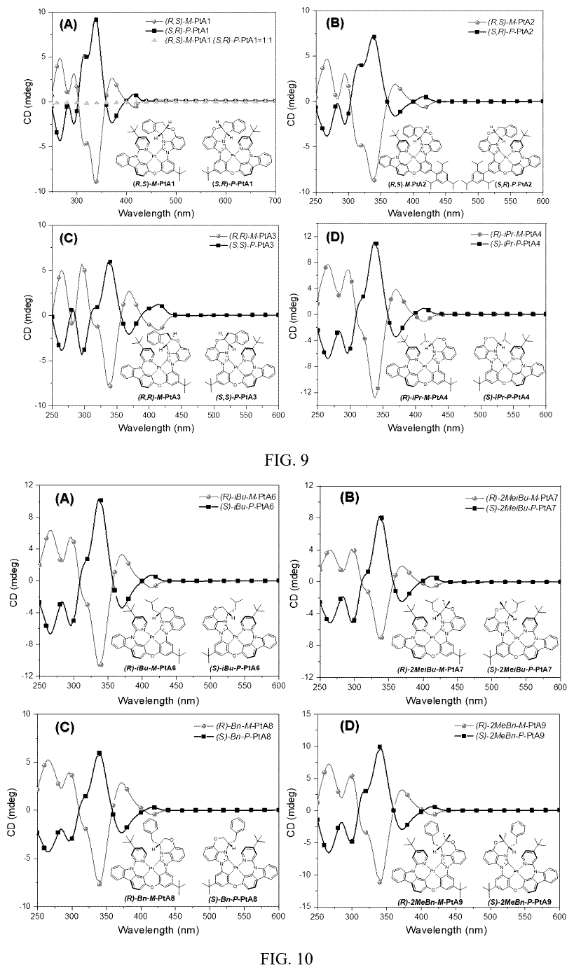

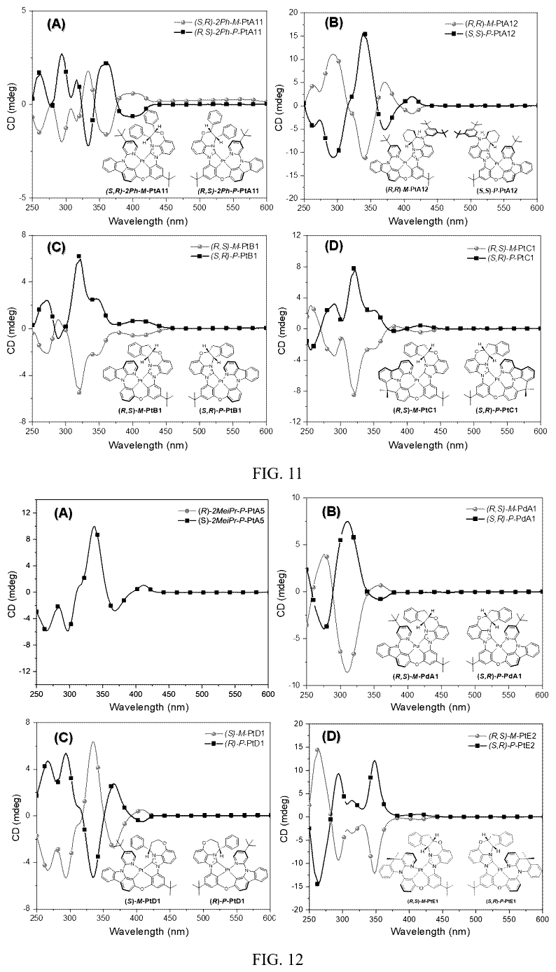

From the circular dichroic (CD) spectra of some material molecules in dichloromethane solution in FIGS. 9 to 12, it can be seen that their spectra have extremely high mirror symmetry, indicating that they are enantiomers with strong deflection ability to straight incident light, and their deflection direction is opposite. For example, the enantiomers (R,S)-M-PtA1 and (S, R)—P—PtA1 exhibit strong Cotton effect responses at approximately 275, 300, and 345 nm, but mixtures prepared with a mass ratio of 1:1 have no response signal.

TABLE 1
Specific rotation ([a]²⁰_{D}) of selected chiral ligands and chiral metal complexes

| | Enantiomeric | Specific | | Enantiomeric | Specific |
|---|---|---|---|---|---|
| compound or | purity (ee | rotation | compound or | purity (ee | rotation |
| metal complex | value) | (°) | metal complex | value) | (°) |
| PtON7-tt | — | 0 | PdON7-tt | — | 0 |
| (comparison | | | (comparison | | |
| complex) | | | complex) | | |
| (R,S)-L1 (ligand) | >99% | +5 | (S,R)-L1 (ligand) | >99% | −8 |
| (R,S)-M-PtA1 | >99% | −585 | (S,R)-P-PtA1 | >99% | +597 |
| (R,S)-M-PtA2 | >99% | −516 | (S,R)-P-PtA2 | >99% | +486 |
| (R,R)-L3 (ligand) | >99% | −7 | (S,S)-L3 (ligand) | >99% | +12 |
| (R,R)-M-PtA3 | >99% | −401 | (S,S)-P-PtA3 | >99% | +424 |
| (R)-iPr-M-PtA4 | >99% | −285 | (S)-iPr-P-PtA4 | >99% | +262 |
| (R)-2MeiPr-M-PtA5 | >99% | −220 | (S)-2MeiPr-P-PtA5 | >99% | +228 |
| (R)-iBu-M-PtA6 | >99% | −259 | (S)-iBu-P-PtA6 | >99% | +270 |
| (R)-2MeiBu-M-PtA7 | >99% | −246 | (S)-2MeiBu-P-PtA7 | >99% | +228 |
| (R)-Bn-M-PtA8 | >99% | −71 | (S)-Bn-P-PtA8 | >99% | +83 |
| (R)-2MeBn-M-PtA9 | >99% | −237 | ((S)-2MeBn-P-PtA9 | >99% | +259 |
| (R)-Ph-M-PtA10 | >99% | −244 | (S)-Ph-P-PtA10 | >99% | +258 |
| (S,R)-2Ph-M-PtA11 | >99% | −4 | (R,S)-2Ph-P-PtA11 | >99% | +9 |
| (R,R)-M-PtA12 | >99% | −145 | (S,S)-P-PtA12 | >99% | +178 |
| (R,S)-M-PdA1 | >99% | −610 | (R,S)-P-PdA1 | >99% | +590 |
| (R,S)-M-PtB1 | >99% | −663 | (R,S)-P-PtB1 | >99% | +655 |
| (R,S)-M-PtC1 | >99% | −703 | (R,S)-P-PtC1 | >99% | +698 |
| (S)-M-PtD1 | >99% | −50 | (R)-P-PtD1 | >99% | +72 |
| (R,S)-M-PtE1 | >99% | −802 | (S,R)-P-PtE1 | >99% | +756 |
| (R,S)-Acr-M-PtF4 | >99% | −680 | (S,R)-Acr-P-PtF4 | >99% | +688 |
| (R)-Me-M-PtA13 | >99% | −238 | (S)-Me-P-PtA13 | >99% | +240 |
| (R)-iPr-M-PtA14 | >99% | −265 | (S)-iPr-P-PtA14 | >99% | +278 |
| (R)-tBu-M-PtA15 | >99% | −291 | (S)-tBu-P-PtA15 | >99% | +303 |
| (R,R)-cyh-M-PtA16 | >99% | −366 | (S,S)-cyh-P-PtA16 | >99% | +363 |
| | | | (S)-CS-P-PtA17 | >99% | +279 |
| (R)-iPr-M-PtA18 | >99% | −248 | (S)-iPr-P-PtA18 | >99% | +268 |
| (R)-tBu-M-PtA19 | >99% | −248 | (S)-tBu-P-PtA19 | >99% | +236 |
| (R)-iPr-M-PtA20 | >99% | −291 | (S)-iPr-P-PtA20 | >99% | +301 |
| (R)-iPr-M-PtA21 | >99% | −371 | (S)-iPr-P-PtA21 | >99% | +372 |
| (R)-iPr-M-PtA22 | >99% | −127 | (S)-iPr-P-PtA22 | >99% | +115 |
| (R)-iBu-M-PtA23 | >99% | −277 | (S)-iBu-P-PtA23 | >99% | +278 |
| (R)-iBu-M-PtA24 | >99% | −322 | (S)-iBu-P-PtA24 | >99% | +338 |
| (R,S)-Tjy-M-PtA25 | >99% | −307 | (S,R)-Tjy-P-PtA25 | >99% | +300 |
| (R,S)-Tjy-M-PtA26 | >99% | −131 | (S,R)-Tjy-P-PtA26 | >99% | +138 |
| | | | (S)-iBu-P-PtA27 | >99% | +377 |
| | | | (S)-iPr-P-PtA28 | >99% | +324 |
| | | | (S,R)-Tjy-P-PtA29 | >99% | +267 |

Note:
The specific rotation values of all samples were measured in dichloromethane solution.

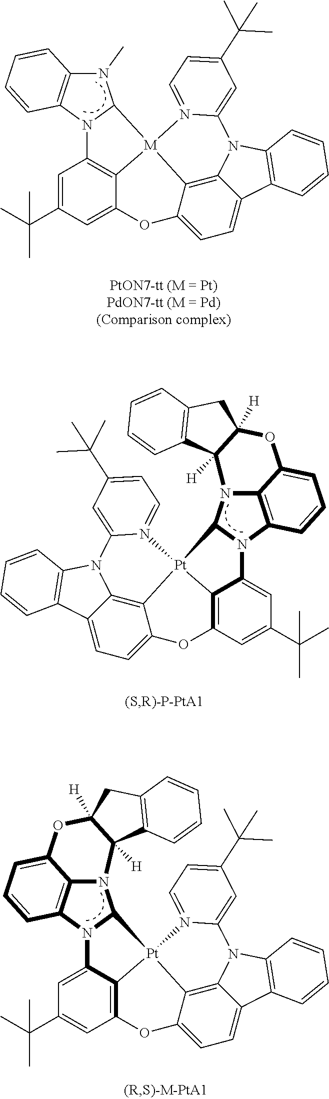

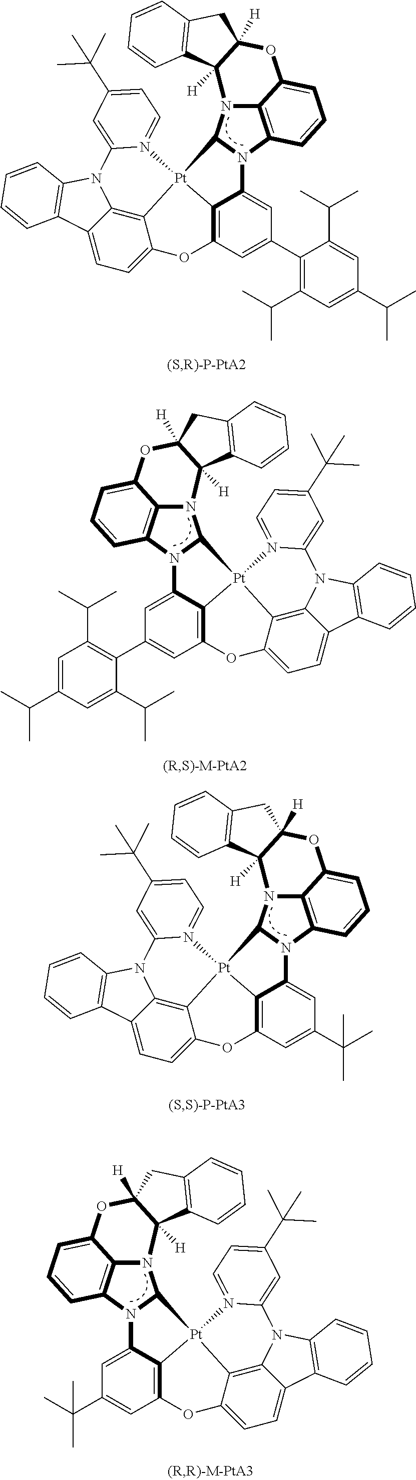

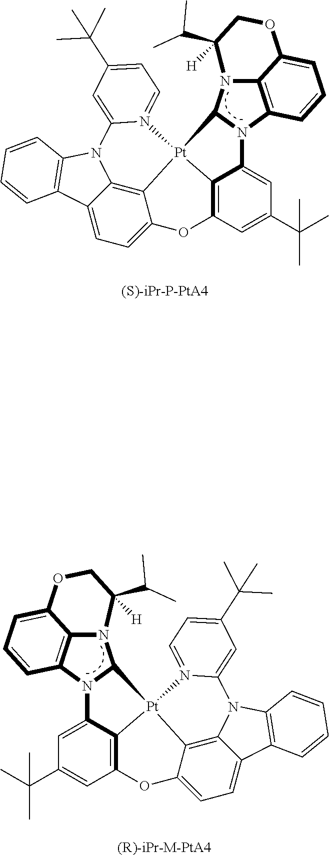

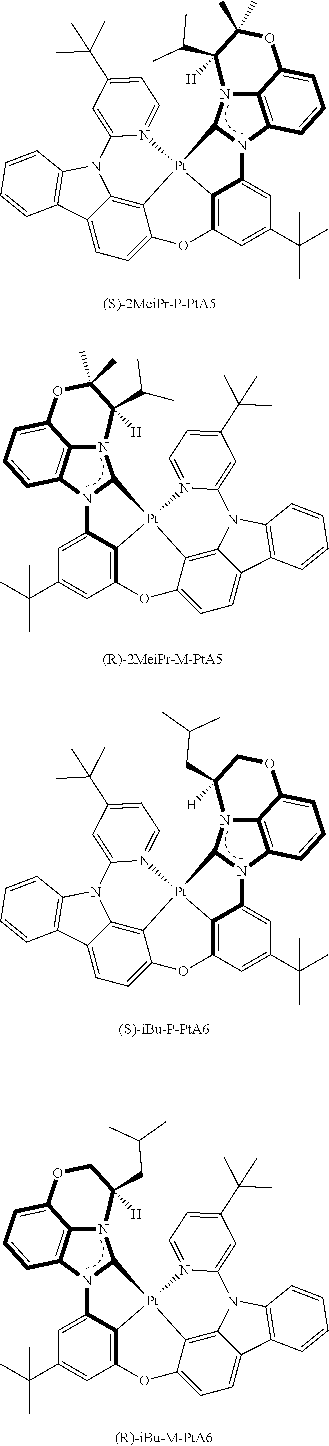

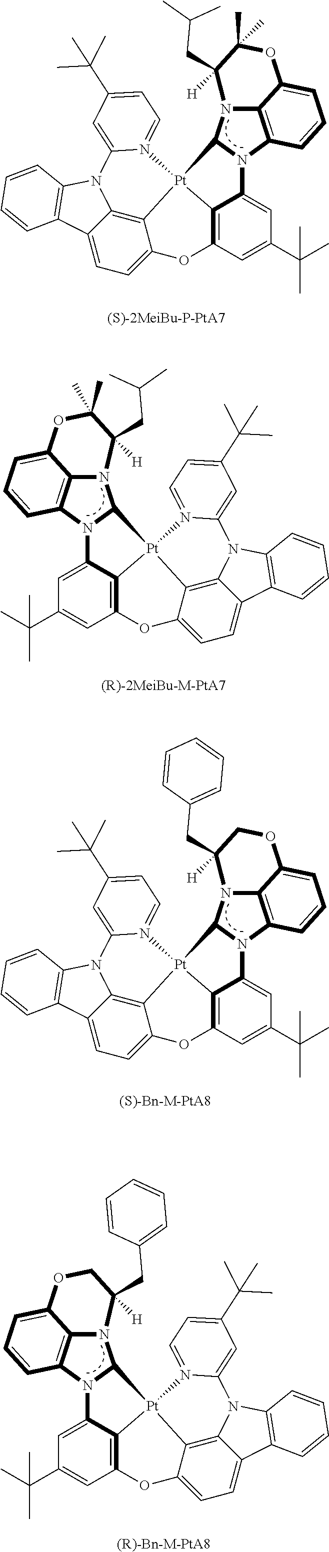

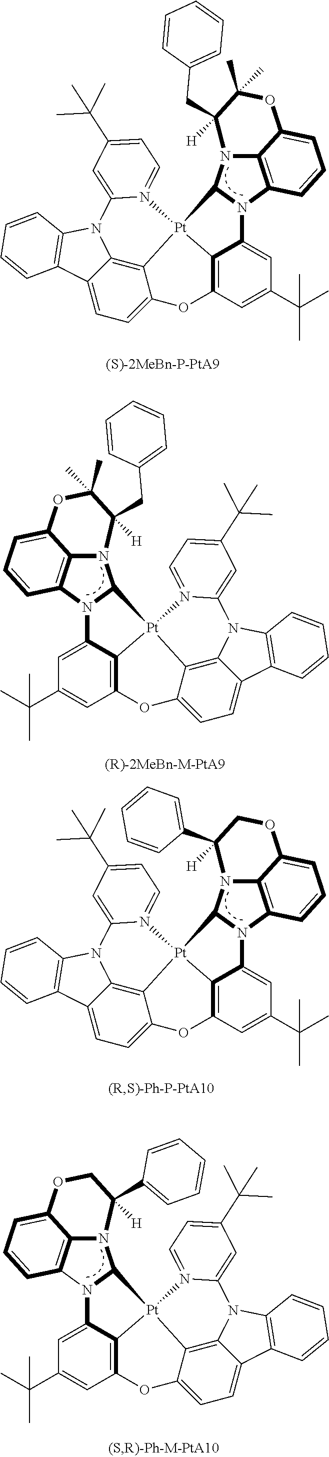

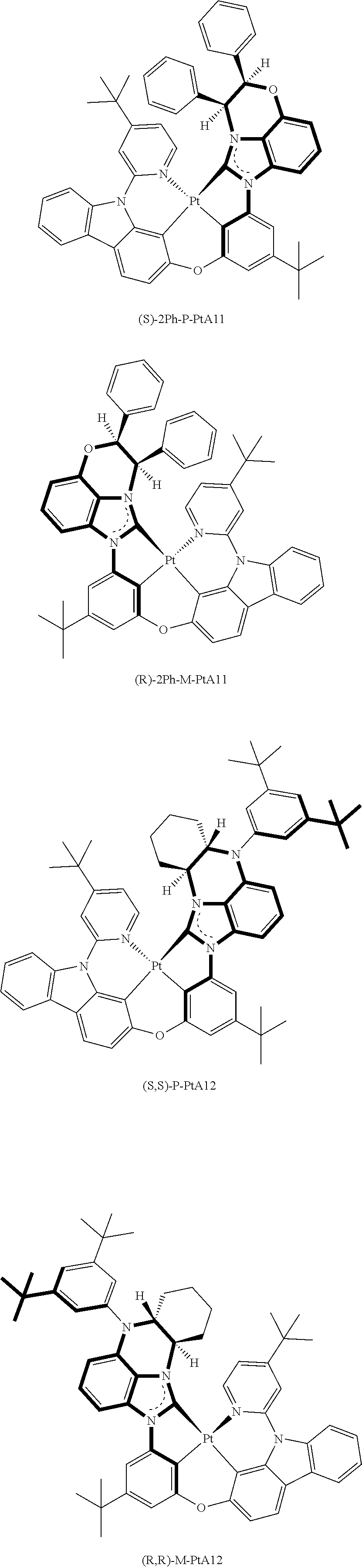

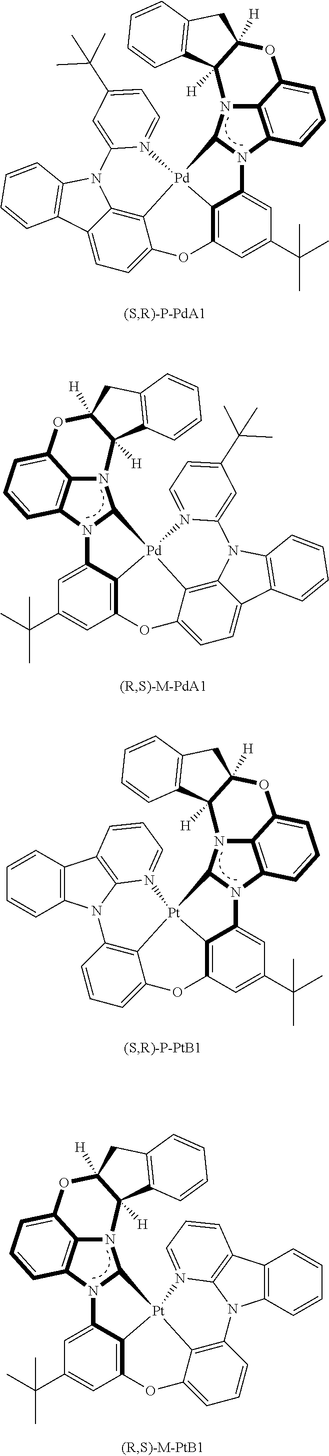

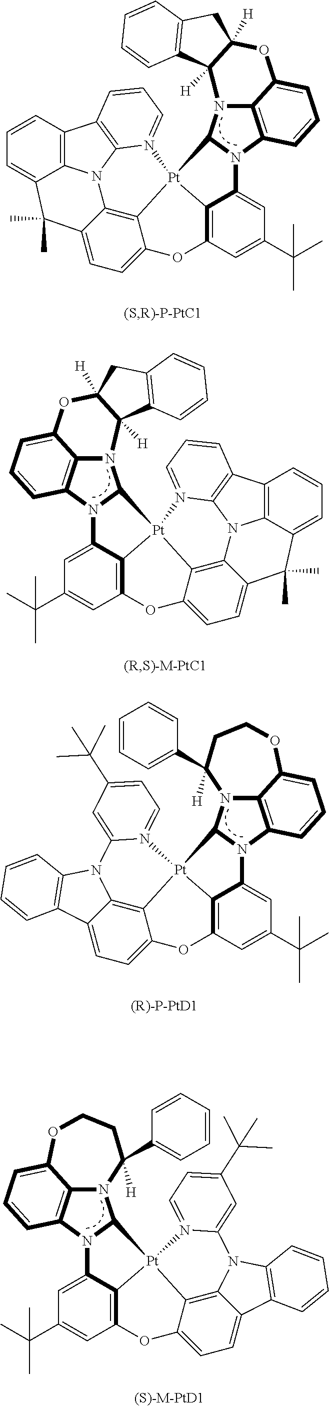

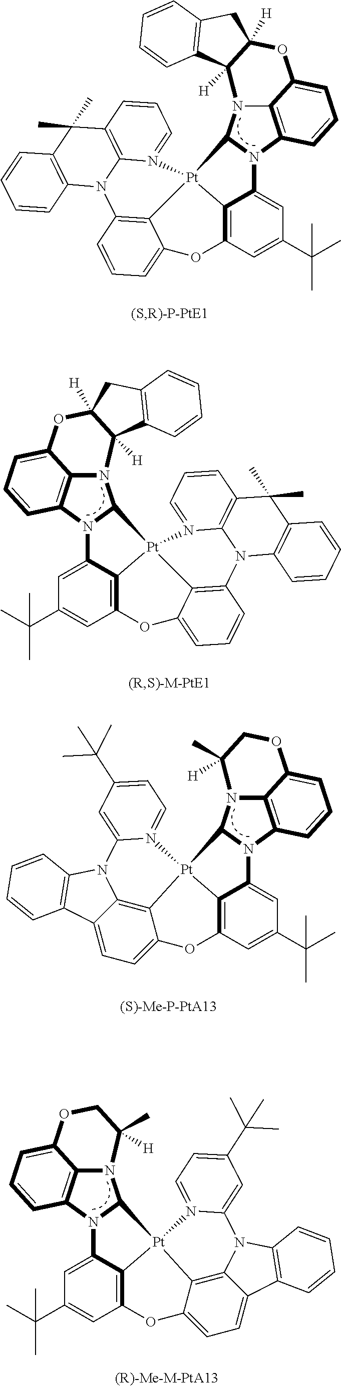

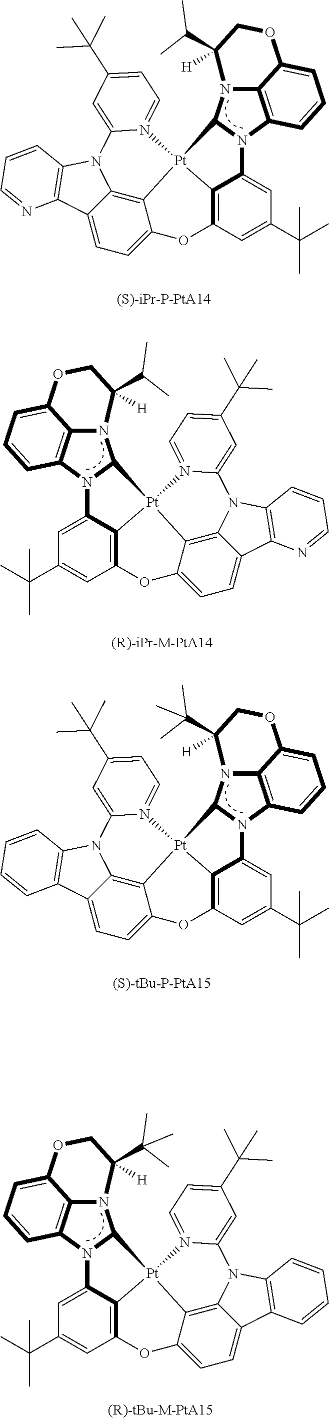

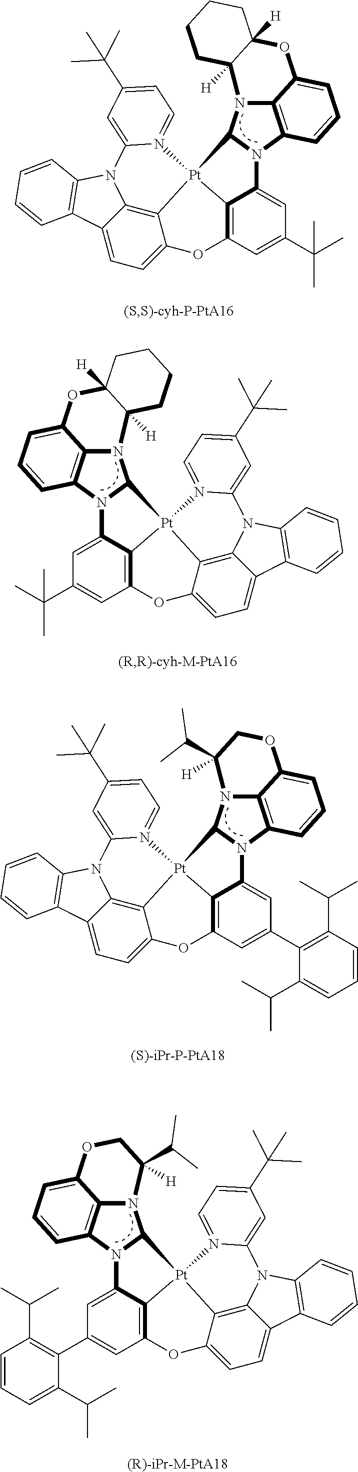

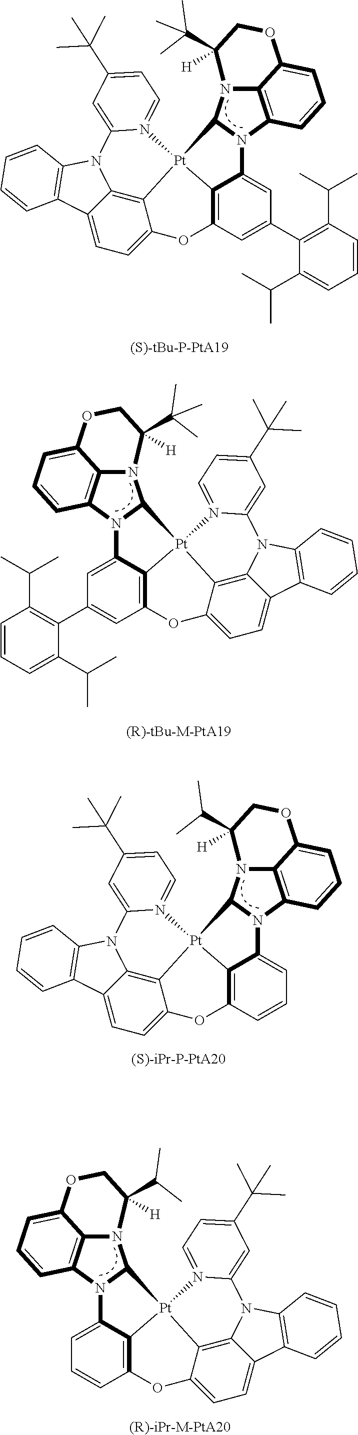

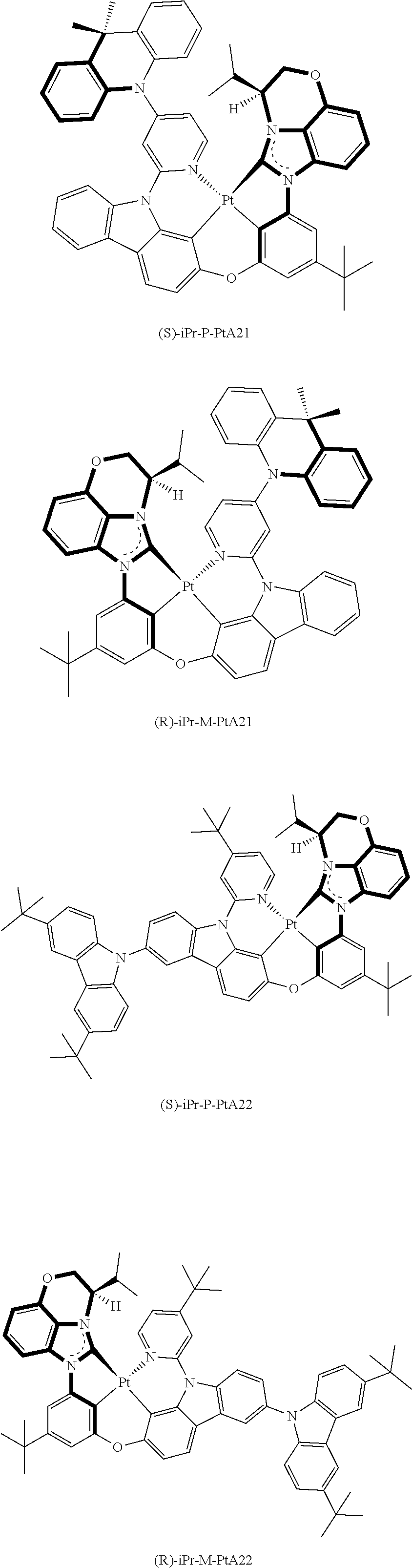

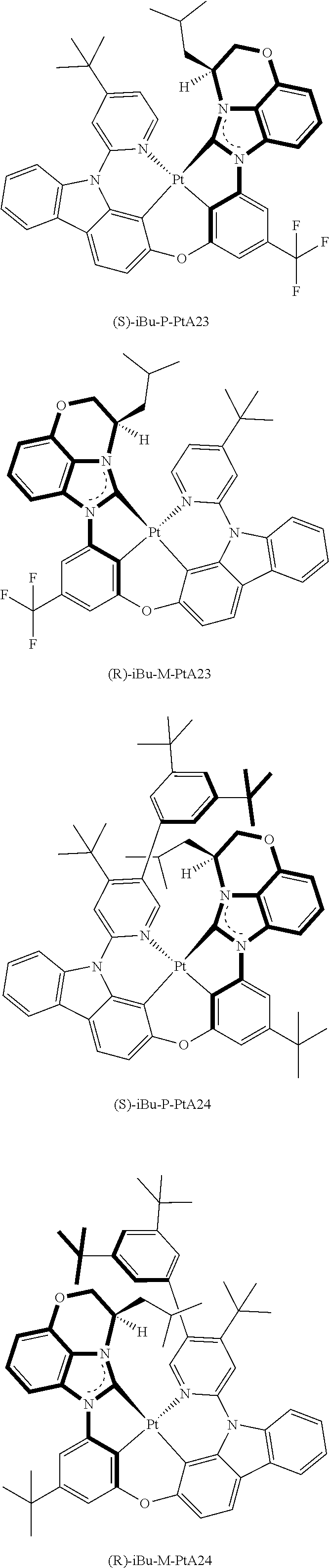

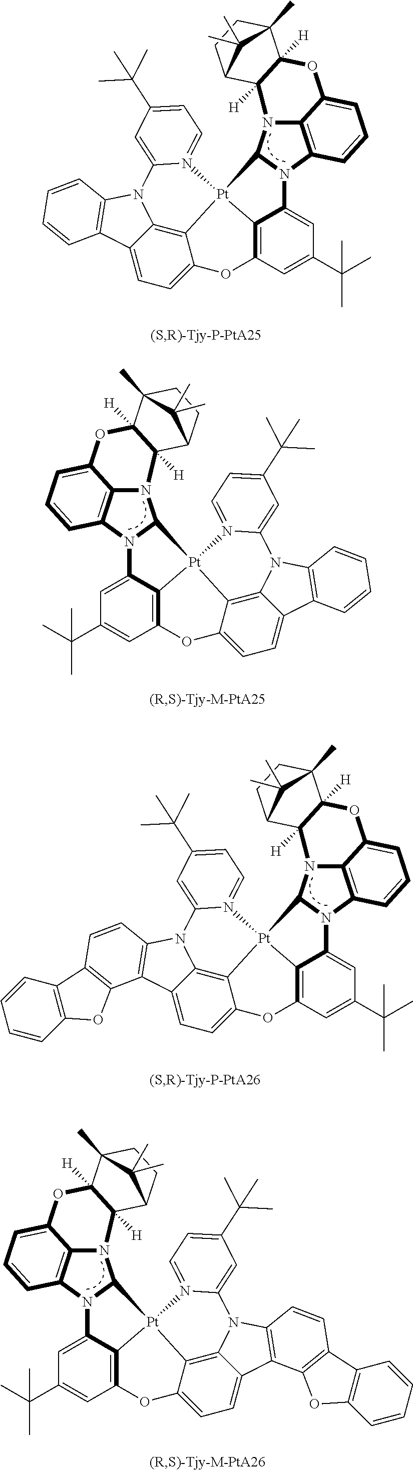

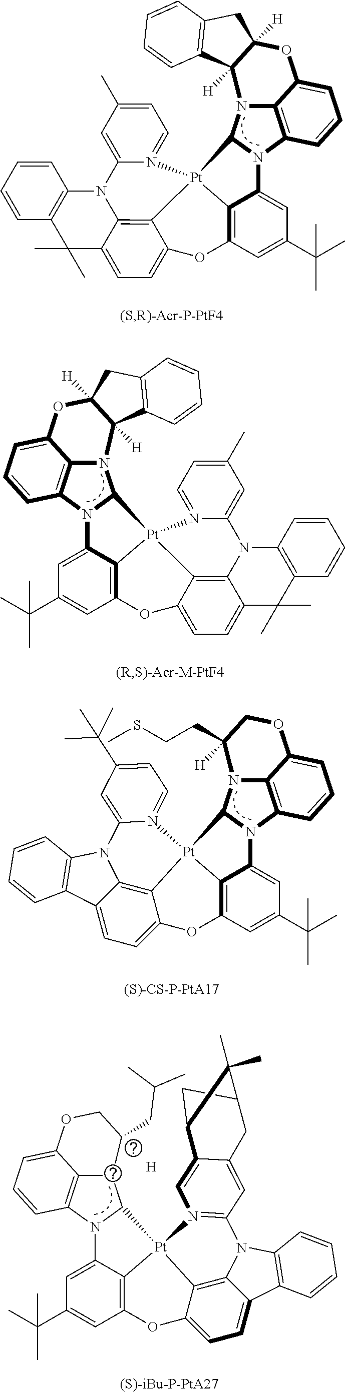

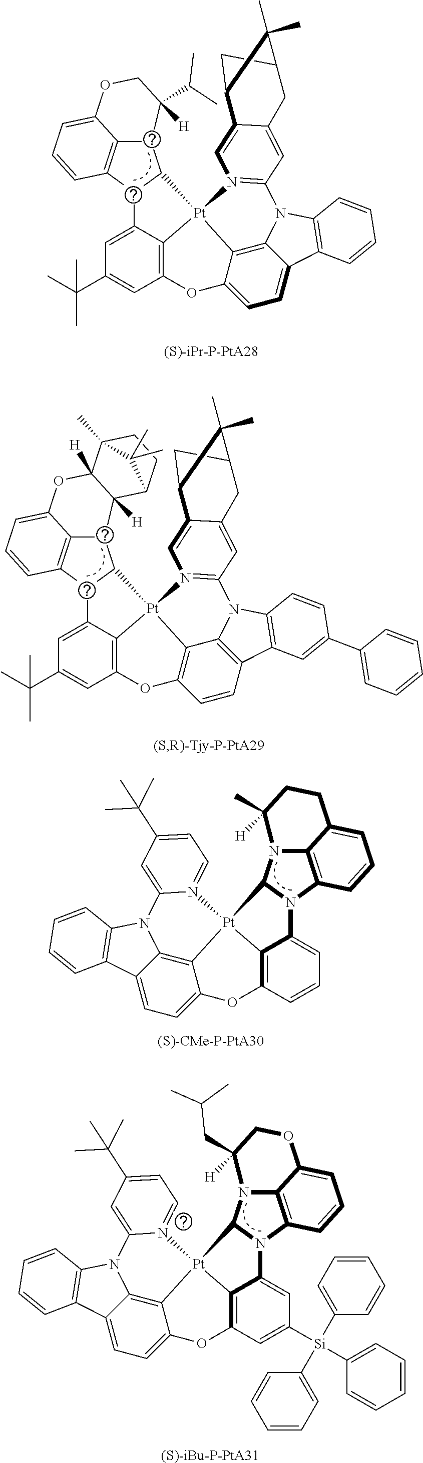

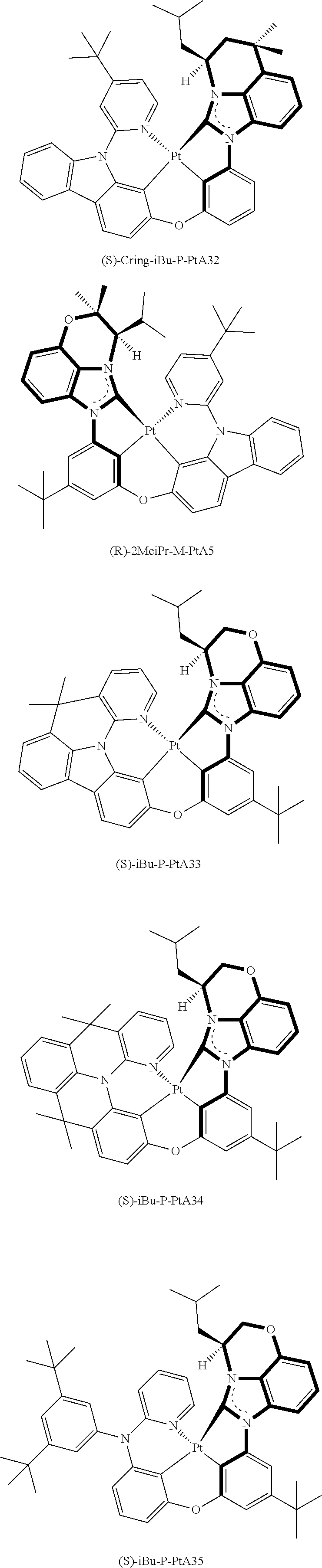

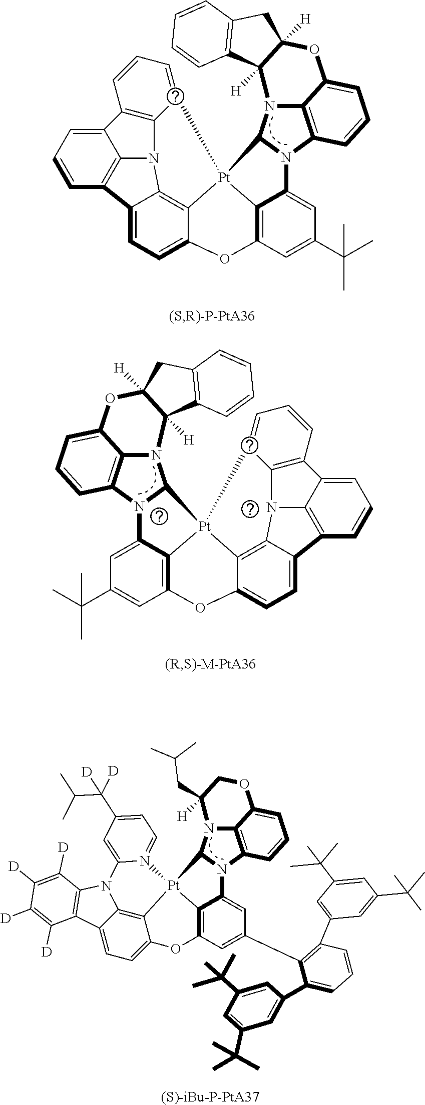

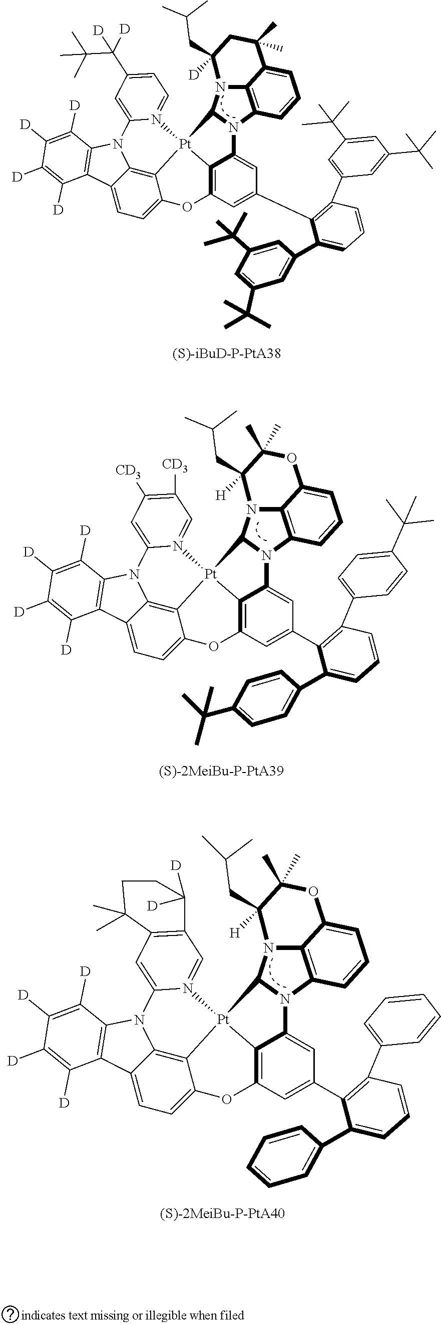

TABLE 2
Maximum emission wavelength (λₘₐₓ) and half width (FWHM) and
dissymmetry factor (g_{PL}) of some selected chiral metal complexes

| | λₘₐₓ | FWHM | g_{PL} | | λₘₐₓ | FWHM | g_{PL} |
|---|---|---|---|---|---|---|---|
| Metal complex | (nm) | (nm) | (×10⁻⁴) | Metal complex | (nm) | (nm) | (×10⁻⁴) |
| PtON7-tt | | | 0 | PdON7-tt | | | 0 |
| (comparison | | | | (comparison | | | |
| complex) | | | | complex) | | | |
| (R,S)-M—PtA1 | 459 | 27 | −7.02 | (S,R)-P—PtA1 | 459 | 27 | +7.00 |
| (R,S)-M—PtA2 | 458 | 24 | −7.59 | (S,R)-P—PtA2 | 458 | 24 | +8.33 |
| (R,R)-M—PtA3 | 463 | 46 | −11.6 | (S,S)-P—PtA3 | 463 | 46 | +10.6 |
| (R)-iPr—M—PtA4 | 456 | 23 | −7.64 | (S)-iPr—P—PtA4 | 456 | 23 | +6.96 |
| (R)-2MeiPr—P—PtA5 | 457 | 28 | −7.32 | (S)-2MeiPr—P—PtA5 | 457 | 28 | +6.98 |
| (R)-iBu—M—PtA6 | 458 | 24 | −8.08 | (S)-iBu—P—PtA6 | 458 | 24 | +8.15 |
| (R)-2MeiBu—M—PtA7 | 457 | 25 | −5.93 | (S)-2MeiBu—P—PtA7 | 457 | 25 | +5.11 |
| (R)-Bn—M—PtA8 | 460 | 26 | −6.51 | (S)-Bn—P—PtA8 | 460 | 26 | +6.81 |
| (R)-2MeBn—M—PtA9 | 462 | 40 | −8.06 | (S)-2MeBn—P—PtA9 | 462 | 40 | +6.64 |
| (R)-Ph—M—PtA10 | 462 | 42 | −4.04 | (S)-Ph—P—PtA10 | 462 | 42 | +3.71 |
| (S,R)-2Ph—M—PtA11 | 459 | 33 | −6.19 | (R,S)-2Ph—P—PtA11 | 459 | 33 | +6.34 |
| (R,R)-M—PtA12 | 458 | 22 | −4.43 | (S,S)-P—PtA12 | 458 | 22 | +3.18 |
| (R,S)-M—PtB1 | 487 | 77 | −1.56 | (R,S)-P—PtB1 | 487 | 77 | +1.62 |
| (R,S)-M—PtC1 | 502 | 63 | −5.34 | (R,S)-P—PtC1 | 502 | 63 | +5.90 |
| (S)-M—PtD1 | 456 | 29 | −7.00 | (R)-P—PtD1 | 456 | 29 | +8.10 |
| (R,S)-M—PtE1 | 458 | 70 | −1.42 | (S,R)-P—PtE1 | 458 | 70 | +1.38 |
| (R,S)-Acr-M—PtF4 | 465 | 54 | −2.13 | (S,R)-Acr-P—PtF4 | 465 | 54 | +2.22 |
| (R)-Me—M—PtA13 | 458 | 44 | −3.20 | (S)-Me—P—PtA13 | 458 | 44 | +3.20 |
| (R)-iPr—M—PtA14 | 457 | 22 | −6.10 | (S)-iPr—P—PtA14 | 457 | 22 | +6.80 |
| (R)-tBu—M—PtA15 | 457 | 24 | −5.83 | (S)-tBu—P—PtA15 | 457 | 24 | +6.62 |
| (R,R)-cyh-M—PtA16 | 461 | 39 | −5.36 | (S,S)-cyh-P—PtA16 | 461 | 39 | +6.44 |
| | | | | (S)-CS-P—PtA17 | 38 | 456 | +6.44 |
| (R)-iPr—M—PtA18 | 456 | 21 | −8.22 | (S)-iPr—P—PtA18 | 456 | 21 | +7.59 |
| (R)-tBu—M—PtA19 | 457 | 25 | −10.17 | (S)-tBu—P—PtA19 | 457 | 25 | +8.43 |
| (R)-iPr—M—PtA20 | 454 | 35 | −9.65 | (S)-iPr—P—PtA20 | 454 | 35 | +7.45 |
| (R)-iPr—M—PtA21 | 492 | 82 | −8.40 | (S)-iPr—P—PtA21 | 492 | 82 | +9.10 |
| (R)-iPr—M—PtA22 | 457 | 22 | −5.70 | (S)-iPr—P—PtA22 | 457 | 22 | +5.95 |
| (R)-iBu—M—PtA23 | 454 | 22 | −9.06 | (S)-iBu—P—PtA23 | 454 | 22 | +9.97 |
| (R)-iBu—M—PtA24 | 459 | 39 | −5.94 | (S)-iBu—P—PtA24 | 459 | 39 | +5.63 |
| (R,S)-Tjy—M—PtA25 | 459 | 24 | −10.19 | (S,R)-Tjy-P—PtA25 | 459 | 24 | +10.28 |
| (R,S)-Tjy—M—PtA26 | 462 | 20 | −7.72 | (S,R)-Tjy-P—PtA26 | 462 | 20 | +7.10 |
| (S)-CMe—P—PtA30 | 450 | 19 | +4.23 | (S)-iBu-P—PtA27 | 459 | 28 | +9.25 |
| (S)-iBu—P—PtA31 | 458 | 26 | +5.31 | (S)-iPr-P—PtA28 | 458 | 26 | +8.30 |
| (S)-Cring—iBu—P—PtA32 | 455 | 23 | +4.86 | (S,R)-Tjy-P—PtA29 | 461 | 23 | +11.76 |
| (S,R)-M—Pt36 | 509 | 65 | −4.62 | (R,S)-P—Pt36 | 506 | 65 | +4.56 |
| (S)-iBu—P—PtA37 | 459 | 24 | +5.23 | (S)-iBuD-P—PtA38 | 459 | 25 | +4.82 |
| (S)-2MeiBu—P—PtA39 | 460 | 25 | +4.31 | (S)-2MeiBu—P—PtA40 | 460 | 26 | +5.43 |

Note:
The maximum emission wavelength (λₘₐₓ) and dssymmetry factor (g_{PL}) of all samples were measured in dichloromethane solution.

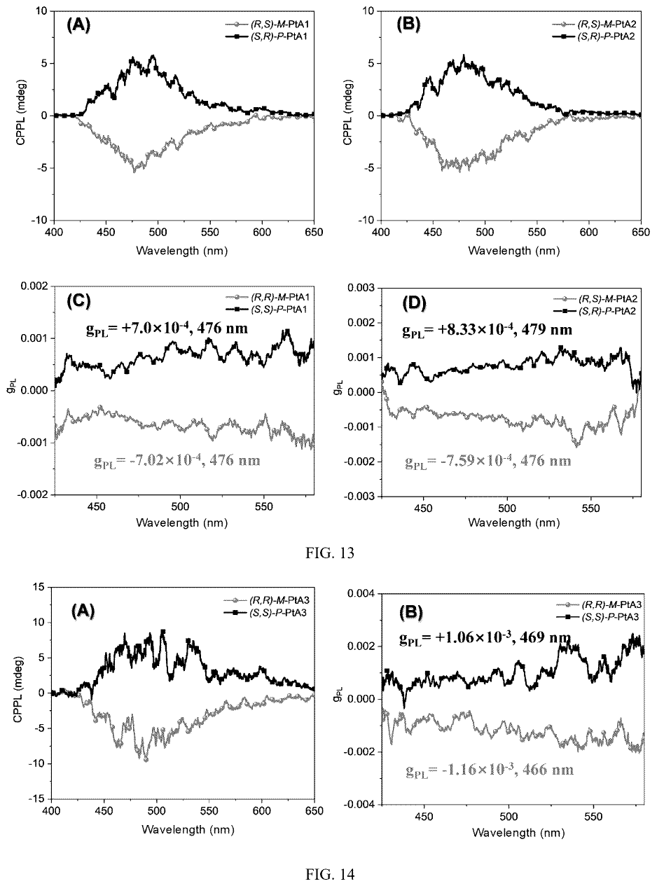

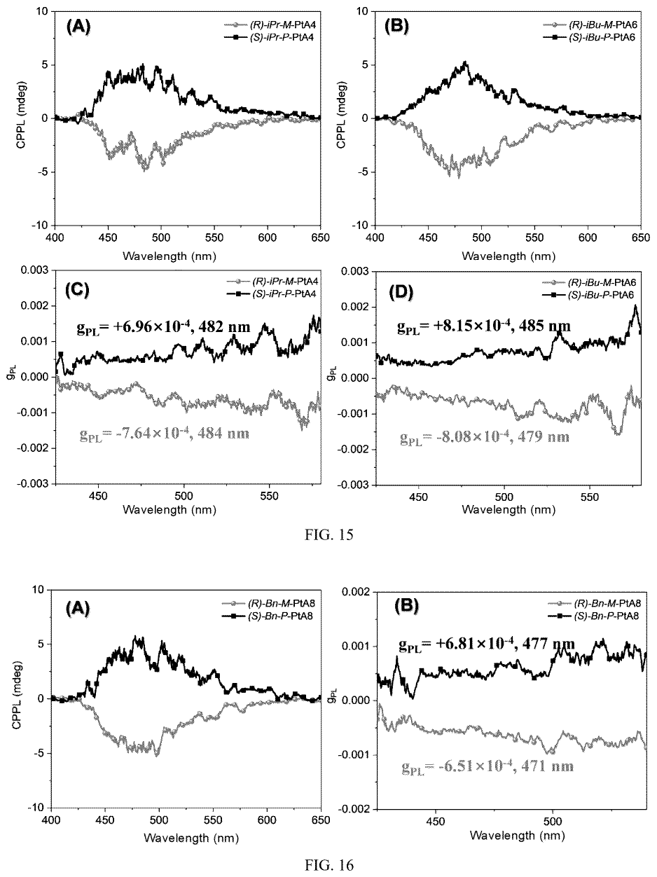

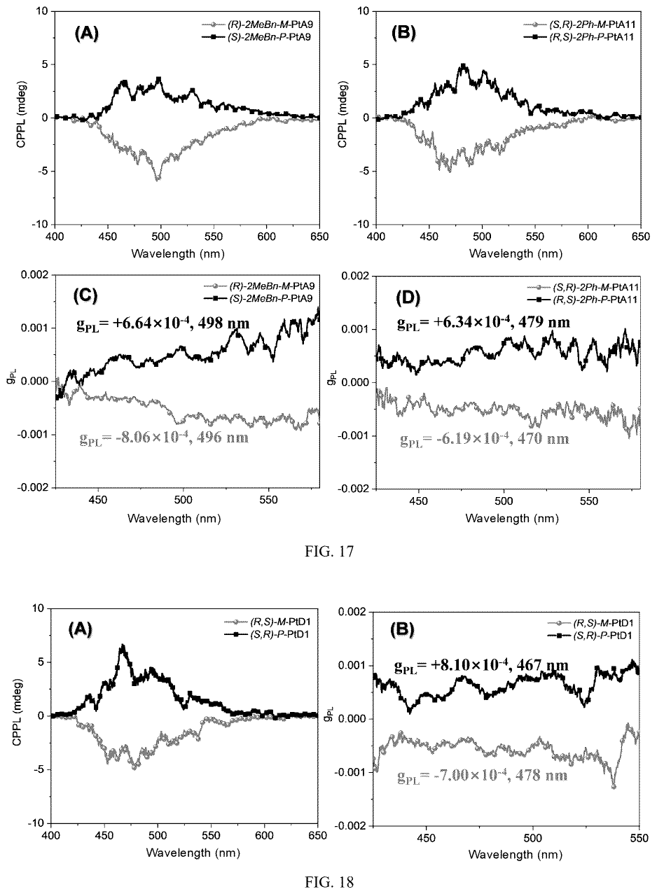

From the circularly polarized photoluminescence spectra (CPPL) of some material molecules in dichloromethane solution in FIGS. 13 to 18, as well as the dissymmetry factor (g_{PL}) curves at different wavelengths, it can be seen that the helically chiral material molecules in the present disclosure exhibit strong circularly polarized luminescence. According to the data in Table 2 above, the absolute value of their dissymmetry factors (g_{PL}) can reach up to 11.6×10⁴. Such helically chiral metal complexes have important applications in circularly polarized luminescent devices and related fields. In contrast, the comparison complexes PtON1-tt and PdON1-tt exhibit no circularly polarized luminescence, and their dissymmetry factors (g_{PL}) are both zero.

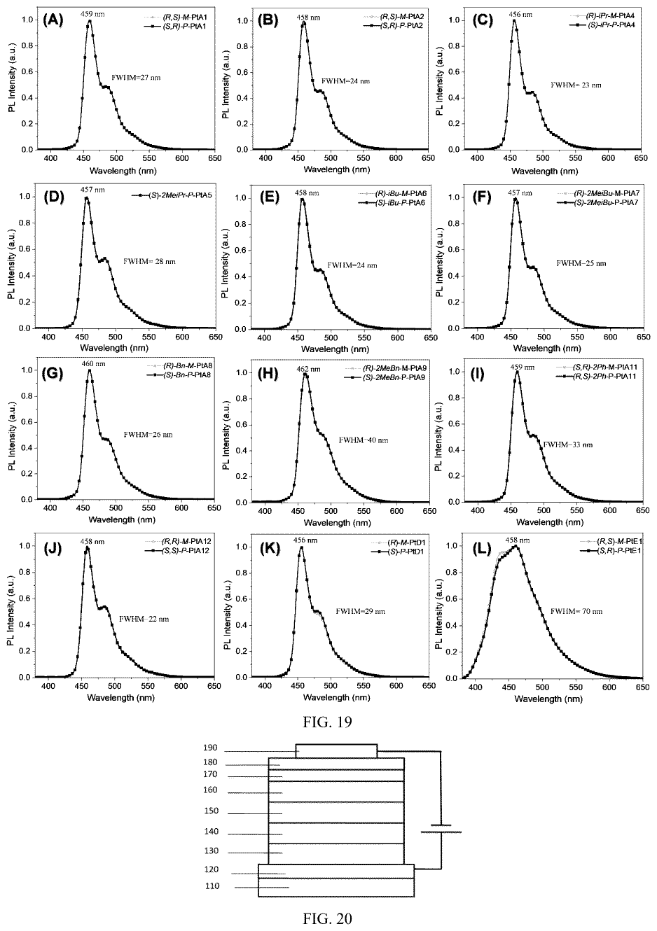

According to the maximum emission wavelength (λₘₐₓ) and half peak width (FWHM) data of helically chiral material molecules in Table 2, as well as their emission spectra in dichloromethane solution in FIG. 19, it can be seen that the majority of helically chiral material molecules in the present disclosure emit narrow band deep blue light (about 460 nm), with very small EWHM values, and high color purity; In addition, tests have shown that their quantum efficiencies are also very high, with the vast majority ranging from 80% to 98%. This type of material molecule is currently urgently needed in the OLED field as a high color purity blue luminescent materials.

In addition, the efficient regulation of the luminescence color of cyclometalated complexes can be achieved through the regulation of the tetradentate ligand structures, which can achieve its regulation from deep blue light to green light, such as the green luminescence of (R,S)-M-PtC1 and its enantiomer (R,S)—P—PtC1 (502 nm).

According to the theoretical calculation experimental data of some helically chiral metal complexes in Table 3, it can be seen that helically chiral molecules based on benzocarbenes and their derivatives with different molecular skeletons and substituents are all twisted quadrilateral structures. The dihedral angles formed between them and the two heterocycles at the end of the central metal ion coordination, i.e. the dihedral angles between benzocarbenes and pyridine rings, are 44.97°-80.32°; The large dihedral angle determines that the four atoms bonded to the central metal are not in the same plane, which can form helically chiral molecules that can be used as circularly polarized luminescent materials. In addition, it can also be seen from Table 3 that the HOMO and LUMO energy levels of helically chiral metal complexes, as well as the energy gap between the HOMO and LUMO, can be efficiently adjusted by adjusting the molecular skeleton structure and its substituents, thereby changing the luminescence wavelength of material molecules. Finally, the large amount of theoretical and experimental data mentioned above also demonstrates the importance of steric hindrance of benzocarbenes and ortho chiral structural units and their functional groups, which is the key to inducing the generation of metal centered spiral chirality in the entire material molecule through ligand centered chirality induction.

In addition, the numerous synthetic experimental embodiments and their characterization and testing of photophysical properties also demonstrate that the molecular design method of circularly polarized luminescent materials in this application is completely successful; these helically chiral metal complexes exhibit strong helically chiral molecular properties, such as CD spectra, specific rotation values, and strongly circularly polarized luminescent signals.

TABLE 3
Theoretical Calculation and Experimental Data
of Molecules of Selected Chiral Metal Complexes

| | LUMO | HOMO | Energy | Dihedral |
|---|---|---|---|---|
| | level | level | gap | angle |
| complex | (eV) | (eV) | (eV) | (°) |
| Pt1 | −1.22 | −4.63 | 3.41 | 63.30 |
| Pt2 | −1.22 | −4.62 | 3.40 | 60.20 |
| Pt3 | −1.27 | −4.64 | 3.37 | 60.40 |
| Pt4 | −1.27 | −4.64 | 3.37 | 58.83 |
| Pt5 | −1.30 | −4.68 | 3.38 | 61.90 |
| Pt6 | −1.28 | −4.63 | 3.35 | 67.56 |
| Pt7 | −1.36 | −4.75 | 3.39 | 60.97 |
| Pt8 | −1.31 | −4.68 | 3.37 | 60.35 |
| Pt9 | −1.22 | −4.63 | 3.41 | 65.99 |
| Pt10 | −1.29 | −4.62 | 3.33 | 66.32 |
| Pt11 | −1.22 | −4.60 | 3.38 | 62.32 |
| Pt12 | −1.39 | −4.72 | 3.33 | 64.84 |
| Pt13 | −1.33 | −4.72 | 3.39 | 50.34 |
| Pt14 | −1.31 | −4.71 | 3.40 | 68.16 |
| Pt15 | −1.33 | −4.67 | 3.34 | 48.72 |
| Pt16 | −1.29 | −4.67 | 3.38 | 73.48 |
| Pt17 | −1.82 | −4.69 | 2.87 | 68.86 |
| Pt18 | −1.23 | −4.62 | 3.39 | 70.62 |
| Pt19 | −1.14 | −4.58 | 3.44 | 61.03 |
| Pt20 | −1.24 | −4.66 | 3.42 | 67.51 |
| Pt21 | −1.56 | −4.69 | 3.13 | 61.33 |
| Pt22 | −1.59 | −4.69 | 3.10 | 56.03 |
| Pt23 | −1.56 | −4.68 | 3.12 | 46.17 |
| Pt24 | −1.59 | −4.69 | 3.10 | 45.86 |
| Pt25 | −1.17 | −4.63 | 3.46 | 58.89 |
| Pt26 | −1.52 | −4.68 | 3..16 | 51.91 |
| Pt27 | −1.18 | −4.84 | 3.66 | 77.13 |
| Pt28 | −1.38 | −5.00 | 3.62 | 78.49 |
| Pt29 | −1.29 | −5.02 | 3.73 | 75.87 |
| Pt30 | −1.23 | −4.94 | 3.71 | 80.32 |
| Pt31 | −1.25 | −4.67 | 3.42 | 65.34 |
| Pt32 | −1.33 | −4.80 | 3.47 | 63.27 |
| Pt33 | −1.26 | −4.60 | 3.34 | 62.35 |
| Pt34 | −1.34 | −4.73 | 3.39 | 64.11 |
| Pt35 | −1.31 | −4.73 | 3.42 | 63.69 |
| Pt36 | −1.34 | −4.72 | 3.38 | 49.21 |
| Pt37 | −1.25 | −4.63 | 3.38 | 50.66 |
| Pt38 | −1.23 | −4.61 | 3.38 | 50.70 |
| Pt39 | −1.99 | −4.72 | 2.73 | 49.38 |
| Pt40 | −1.24 | −4.64 | 3.40 | 49.69 |
| Pt41 | −1.28 | −4.66 | 3.38 | 49.61 |
| Pt42 | −1.27 | −4.67 | 3.40 | 71.04 |
| Pt43 | −1.23 | −4.59 | 3.36 | 48.36 |
| Pt44 | −1.26 | −4.63 | 3.37 | 46.78 |
| Pt45 | −1.28 | −4.59 | 3.31 | 51.12 |
| Pt46 | −1.29 | −4.63 | 3.34 | 49.31 |
| Pt47 | −1.54 | −4.97 | 3.43 | 50.47 |
| Pt48 | −1.28 | −4.73 | 3.45 | 68.00 |
| Pt49 | −1.30 | −4.60 | 3.30 | 44.97 |
| Pt50 | −1.41 | −4.75 | 3.34 | 49.38 |
| Pt51 | −1.31 | −4.70 | 3.39 | 50.38 |
| Pt52 | −1.29 | −4.69 | 3.40 | 52.26 |
| Pt53 | −1.32 | −4.65 | 3.33 | 53.55 |
| Pt54 | −1.35 | −4.67 | 3.32 | 56.59 |
| Pt55 | −1.34 | −4.64 | 3.30 | 51.77 |
| Pt56 | −1.37 | −4.66 | 3.29 | 57.17 |
| Pt57 | −1.20 | −4.67 | 3.47 | 75.31 |
| Pt58 | −1.34 | −4.70 | 3.36 | 50.60 |
| Pt59 | −1.28 | −4.62 | 3.34 | 58.49 |
| Pt60 | −1.28 | −4.65 | 3.37 | 60.91 |
| Pt61 | −1.29 | −4.62 | 3.33 | 56.47 |
| Pd61 | −1.20 | −4.68 | 3.48 | 57.40 |
| Pt62 | −1.03 | −4.55 | 3.52 | 42.41 |
| Pt63 | −1.90 | −4.64 | 2.74 | 48.76 |
| Pt64 | −1.36 | −4.50 | 3.14 | 68.35 |
| Pt65 | −1.50 | −5.00 | 3.5 | 51.78 |
| Pt66 | −1.92 | −4.59 | 2.67 | 43.91 |
| Pt67 | −1.48 | −4.94 | 3.46 | 63.60 |
| Pt68 | −1.53 | −4.75 | 3.22 | 49.79 |
| Pt69 | −1.43 | −4.69 | 3.26 | 34.26 |
| Pt70 | −1.45 | −4.78 | 3.33 | 30.70 |
| Pt71 | −1.46 | −4.77 | 3.31 | 42.67 |
| Pt72 | −1.56 | −4.83 | 3.27 | 33.57 |
| Pt73 | −1.50 | −4.77 | 3.27 | 20.86 |

Note:
Dihedral angle is the angle formed between the two heterocycles at the end of the tetradentate ligand coordination with the central metal ion, i.e. the dihedral angle between the benzocarbene and pyridine rings. Energy gap = LUMO level − HOMO level.

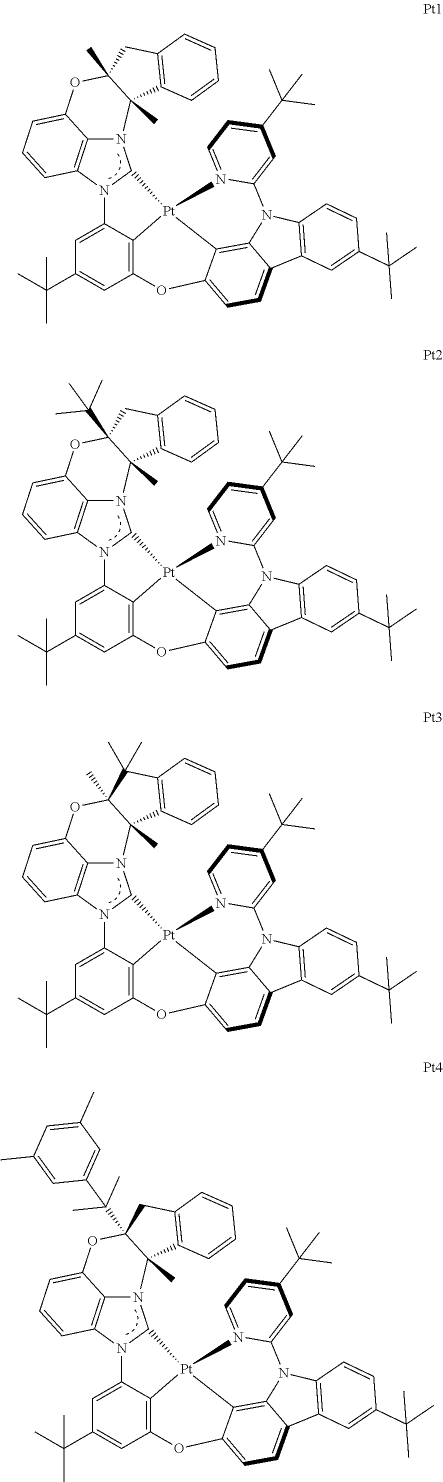

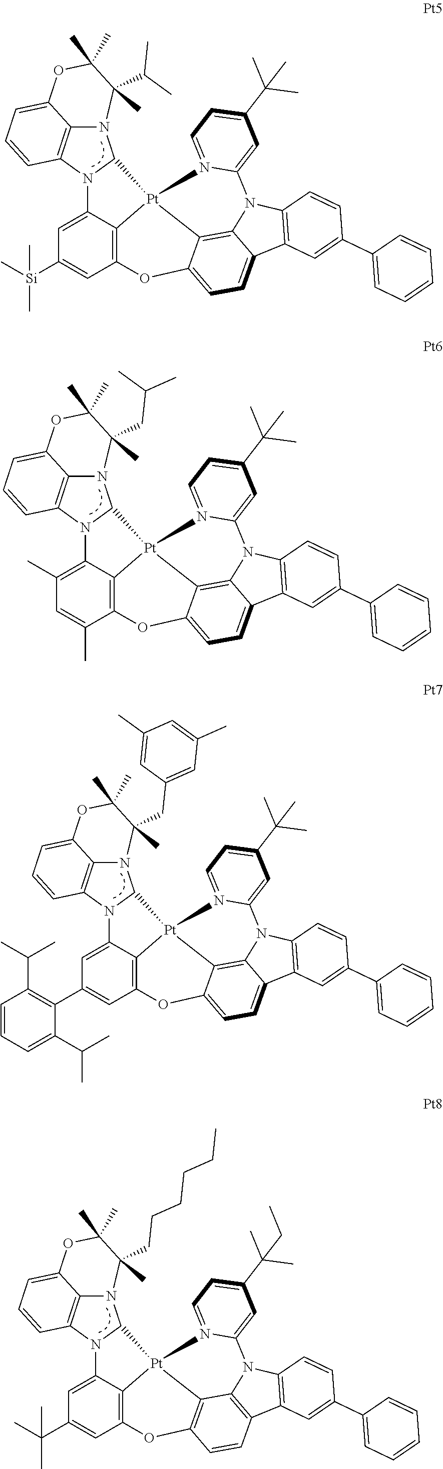

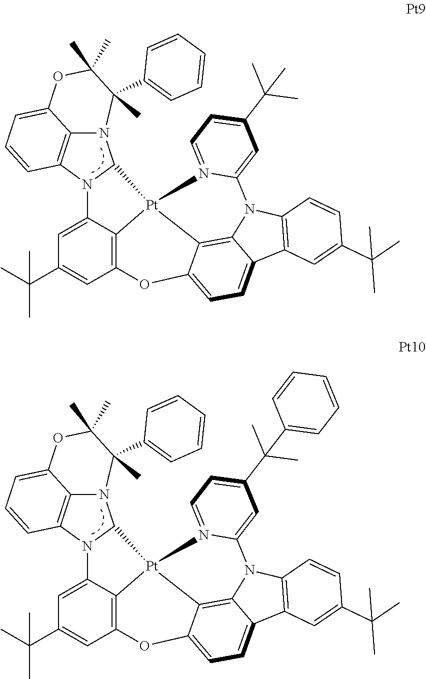

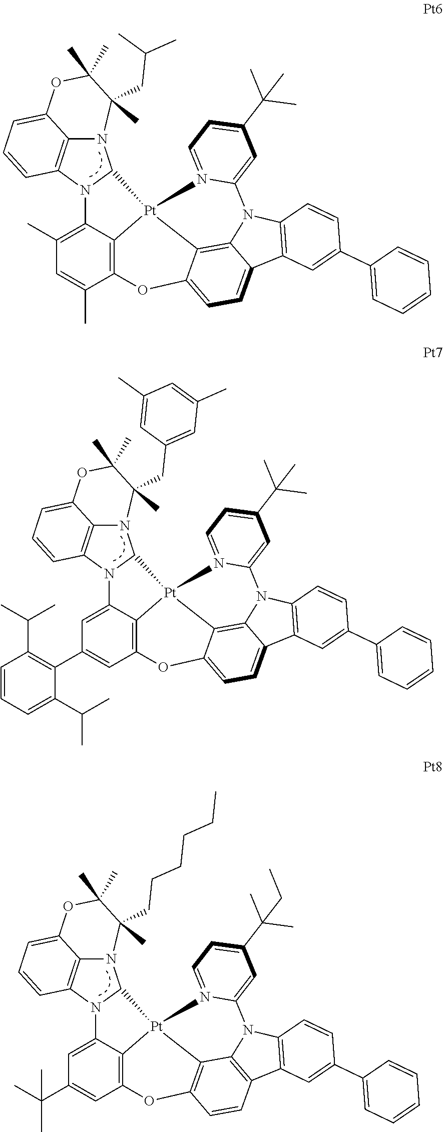

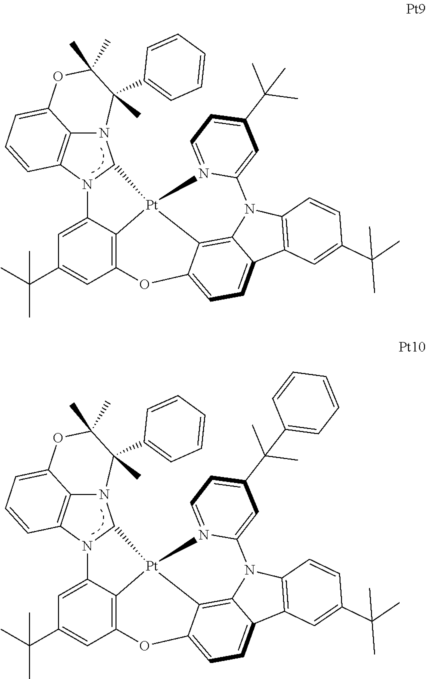

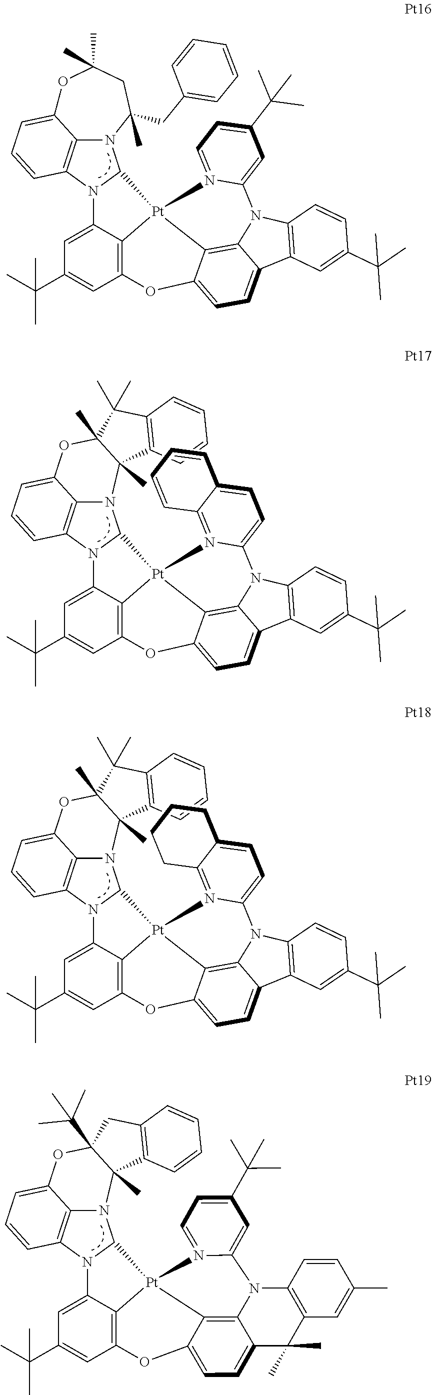

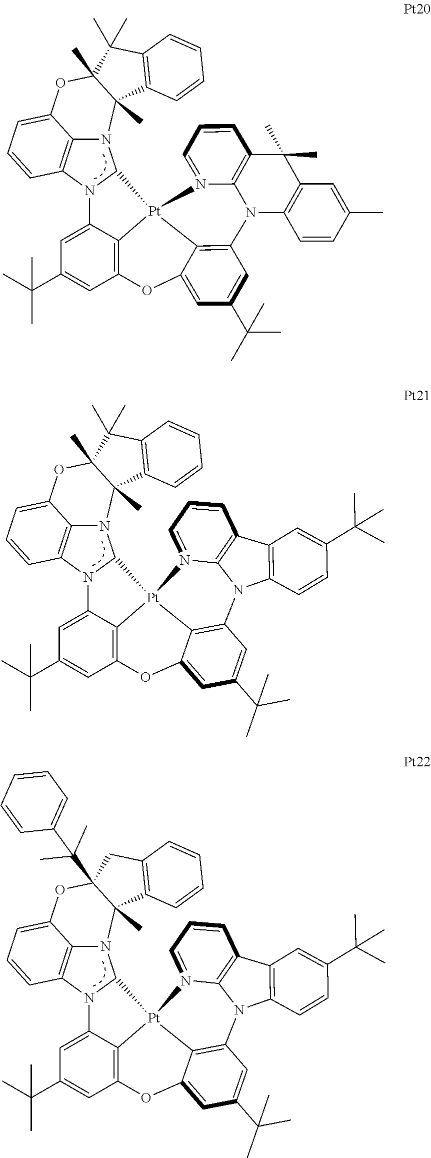

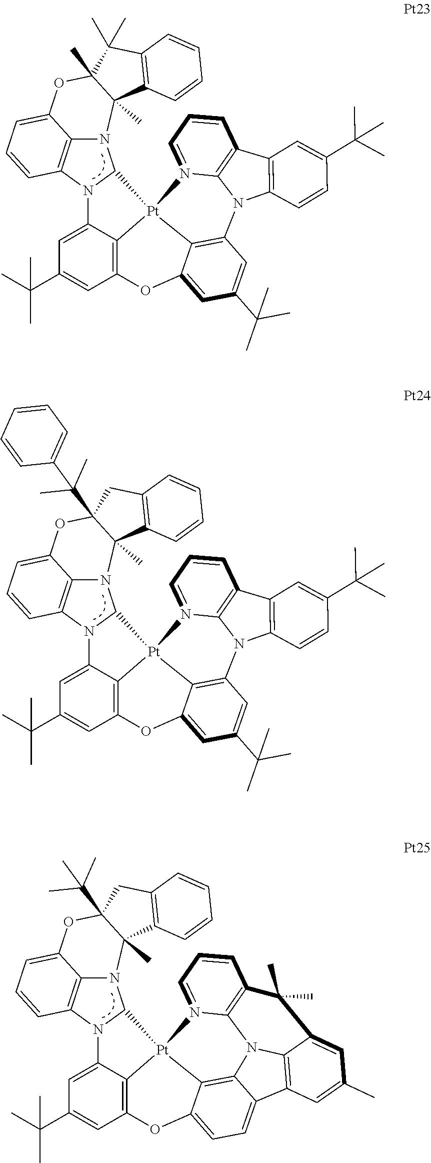

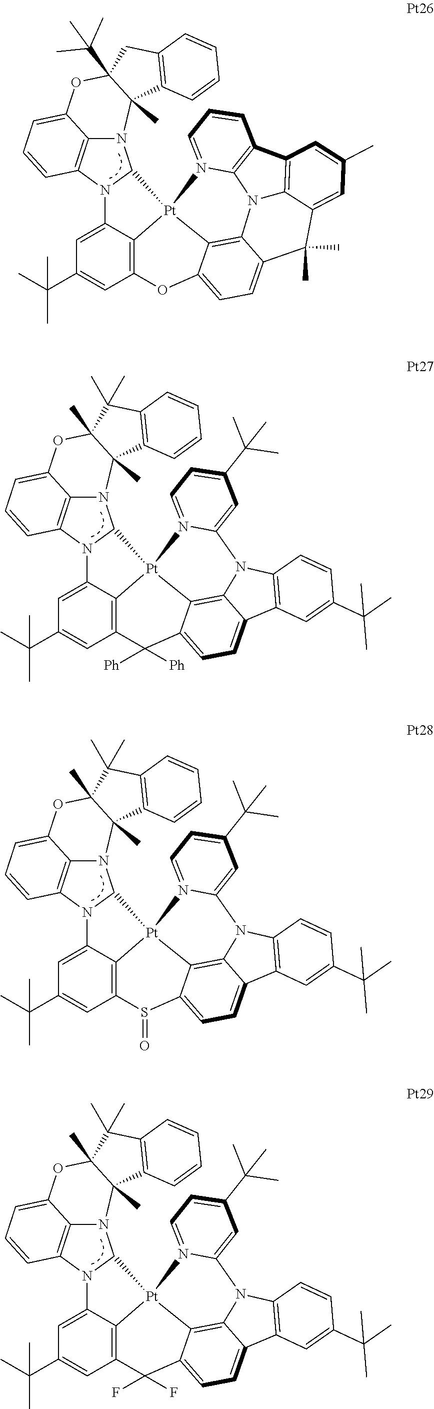

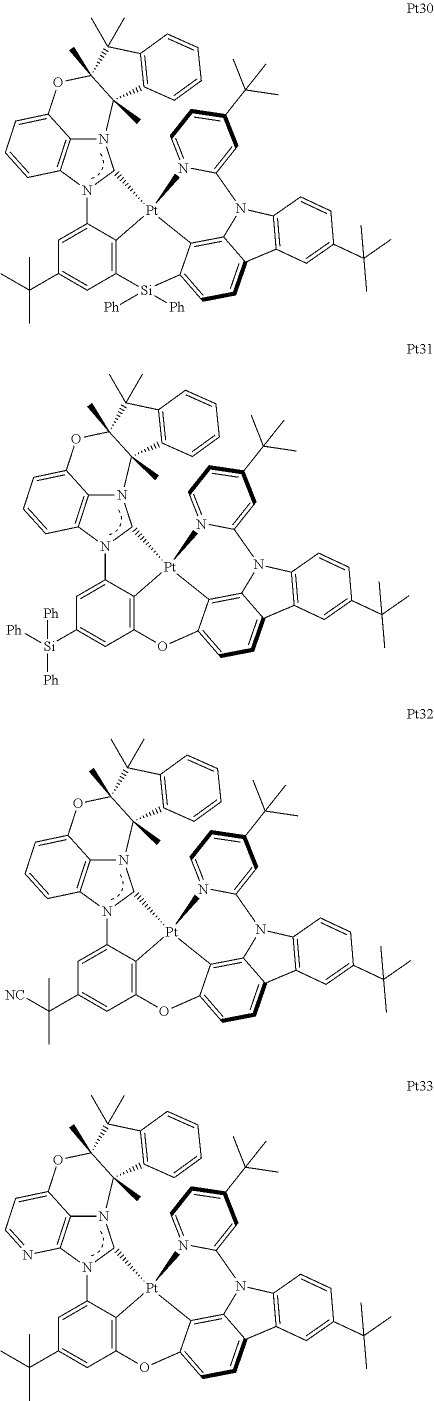

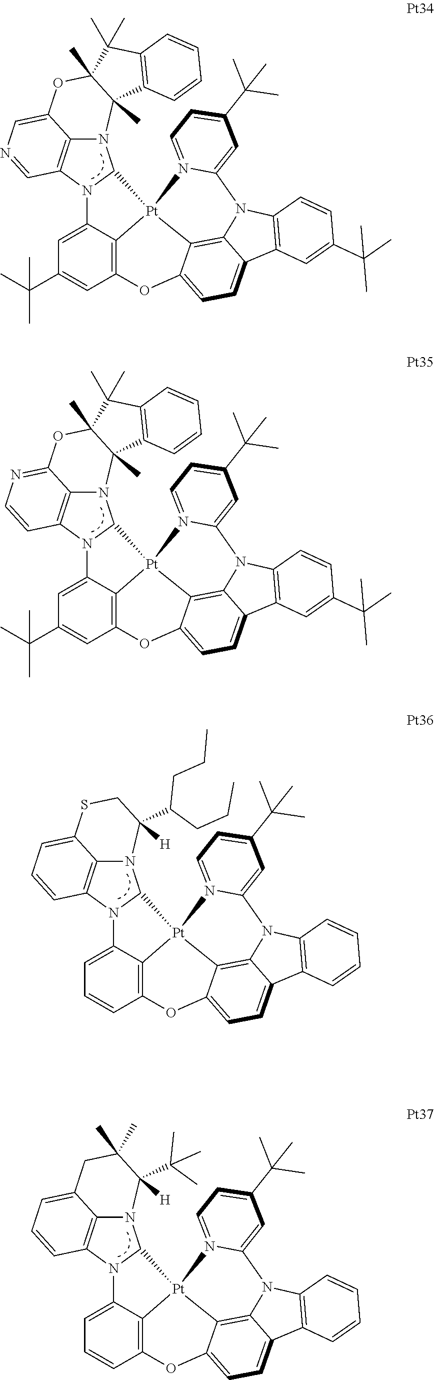

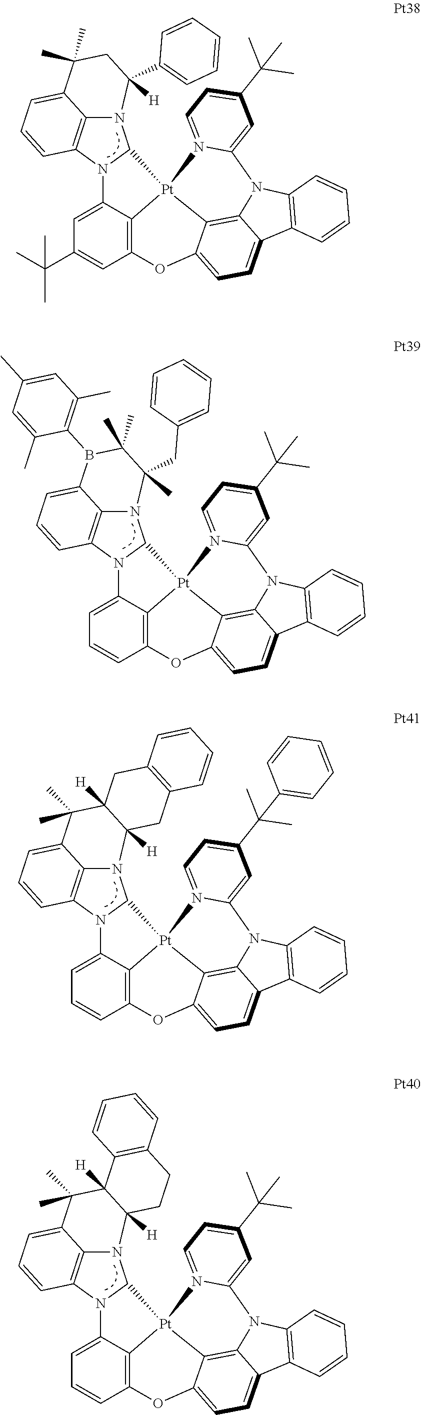

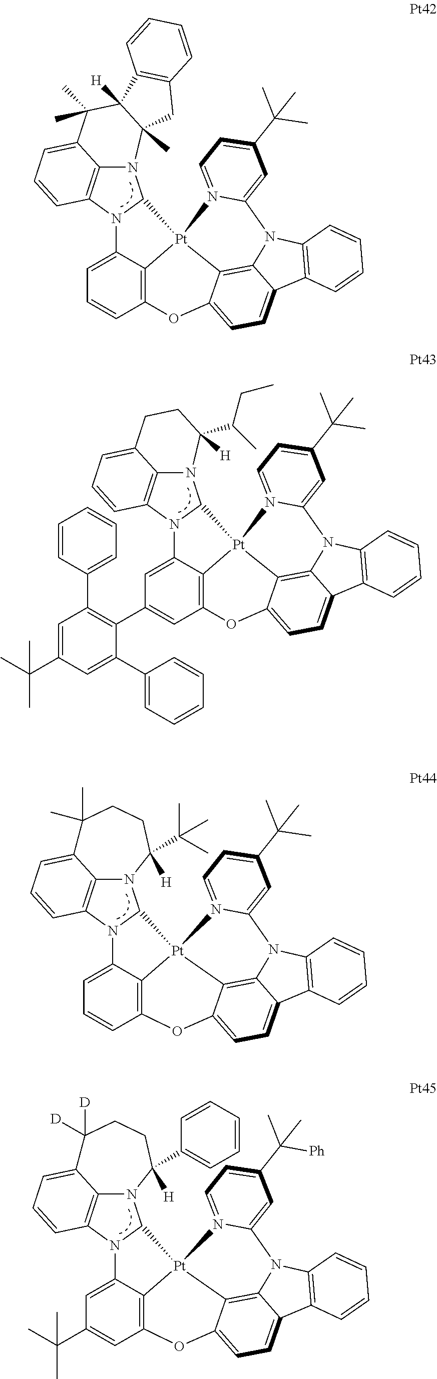

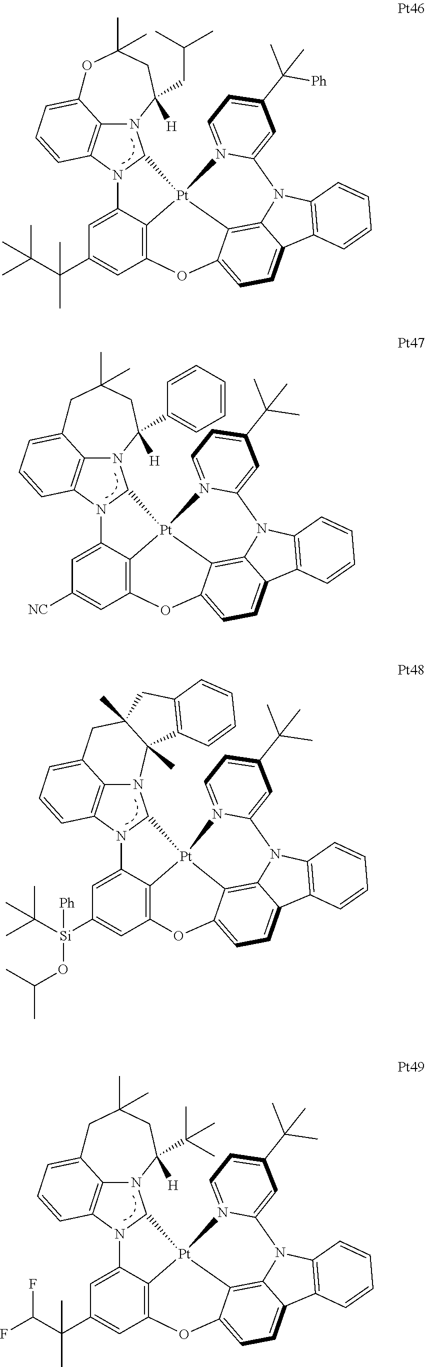

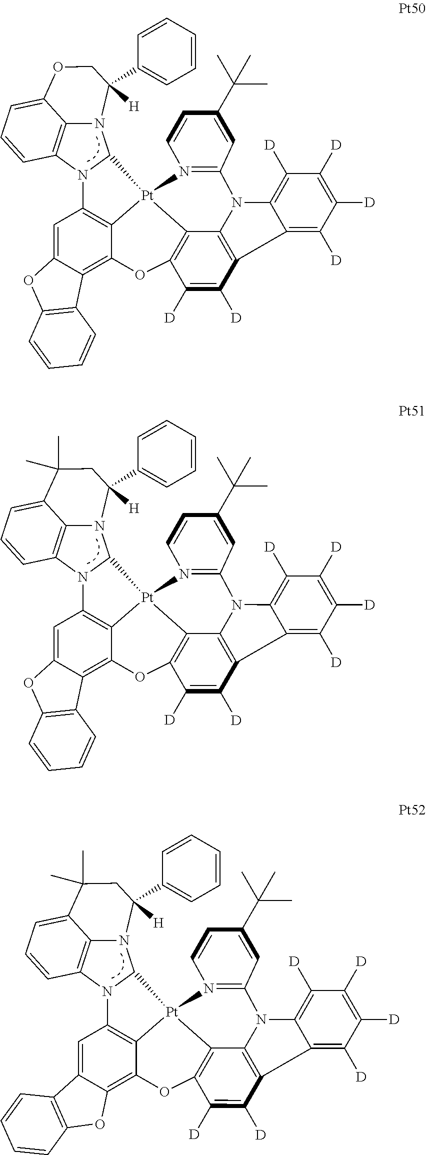

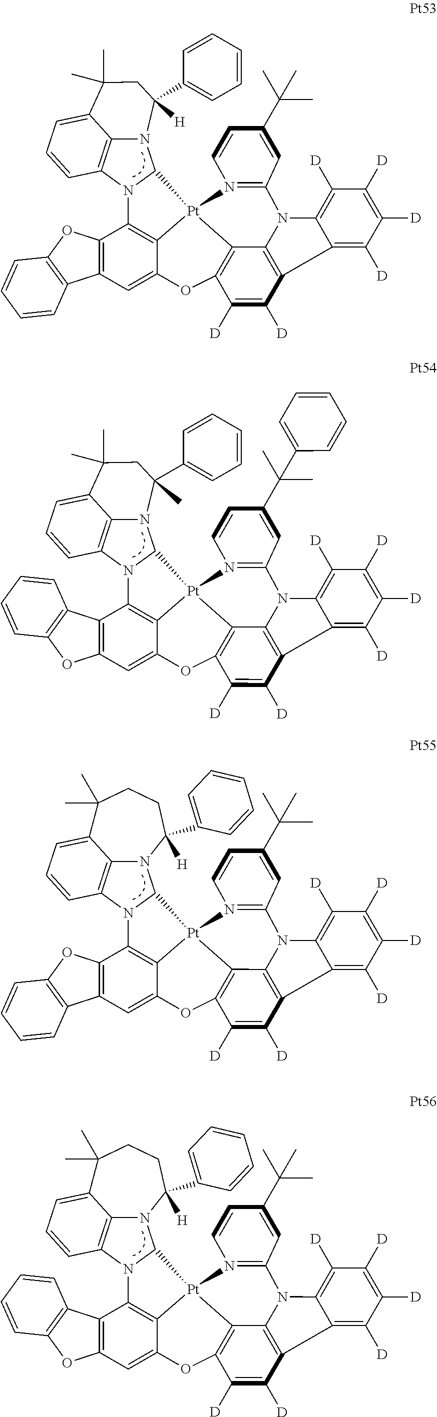

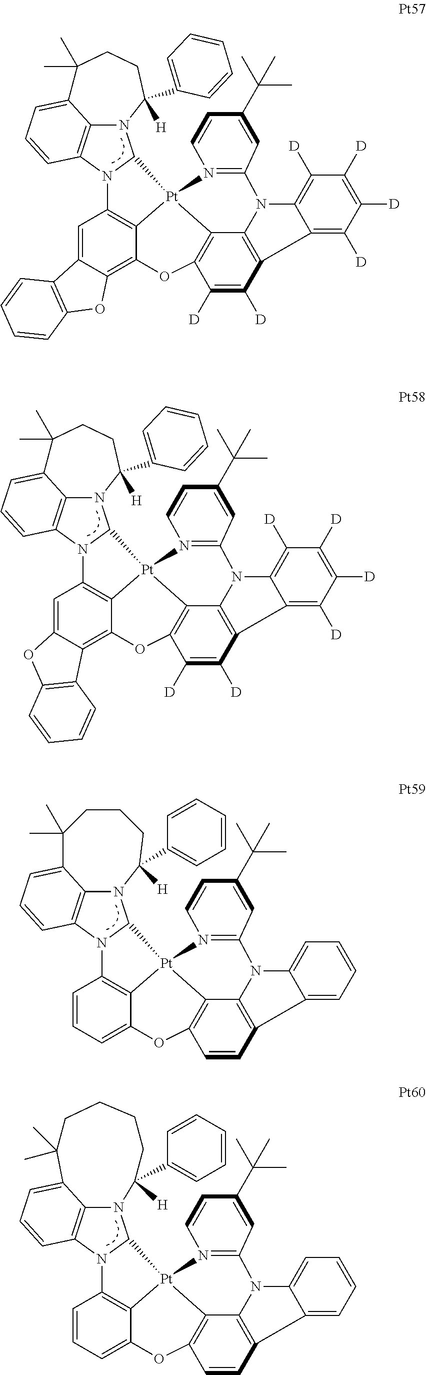

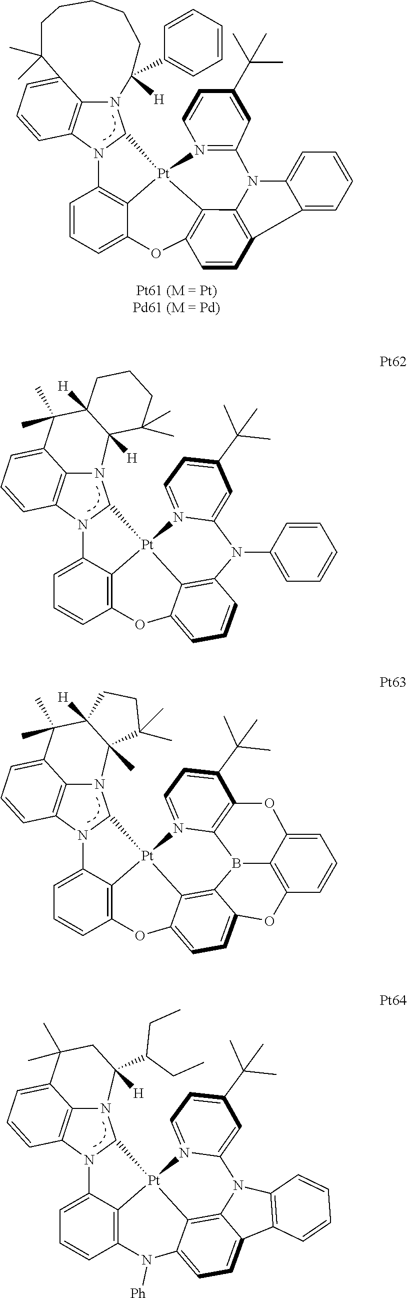

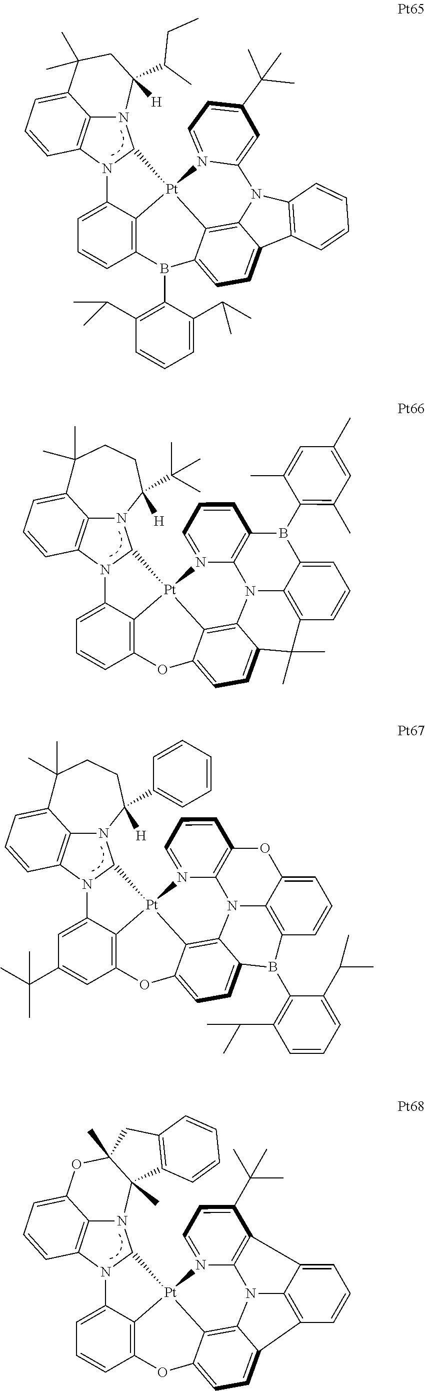

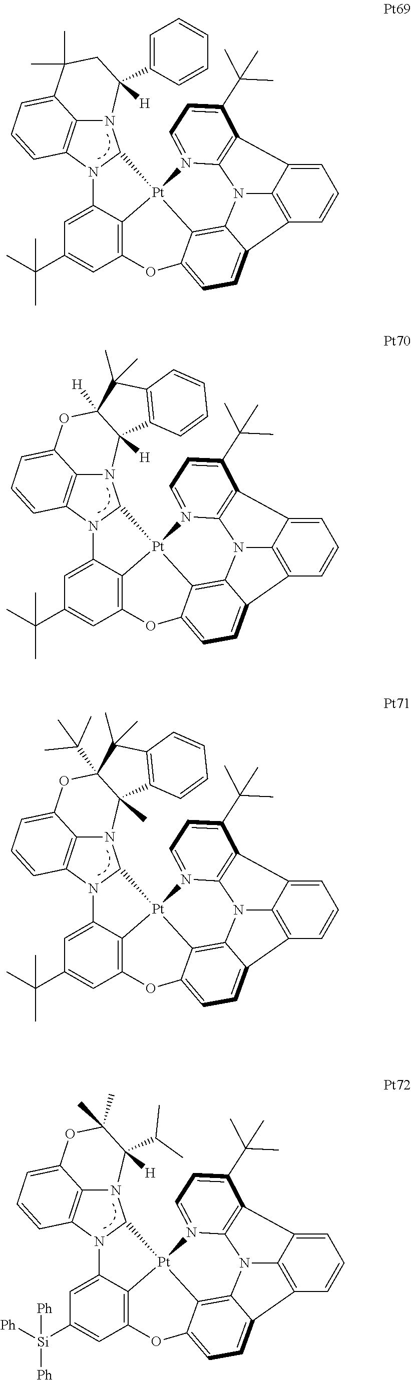

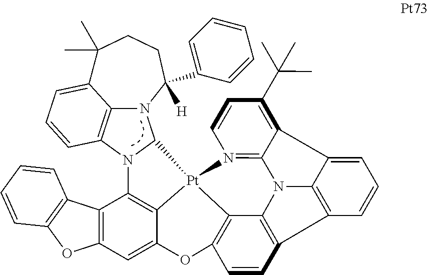

In organic light-emitting devices, carriers are injected into the luminescent material from the positive and negative electrodes to generate excited states of the luminescent material and make it emit light. The complex represented by the general formula (I) of the present disclosure can be applied as a phosphorescent material to excellent organic light-emitting devices such as organic photoluminescence devices or organic electroluminescent devices. Organic photoluminescent devices have a structure that forms at least a luminescent layer on the substrate. In addition, organic electroluminescent devices have a structure that forms at least an anode, cathode, and an organic layer between the anode and cathode. The organic layer at least includes a luminescent layer, which can be composed solely of the luminescent layer or have more than one organic layer in addition to the luminescent layer. As other organic layers, they can be listed as hole transport layer, hole injection layer, electron barrier layer, hole barrier layer, electron injection layer, electron transport layer, exciton barrier layer, etc. The hole transport layer can also be a hole injection transport layer with hole injection function, and the electron transport layer can also be an electron injection transport layer with electron injection function. The specific structure diagram of the organic light-emitting element is shown in FIG. 20. In FIG. 20, 110 represents the substrate, 120 represents the anode, 130 represents the hole injection layer, 140 represents the hole transport layer, 150 represents the luminescent layer, 160 represents the hole blocking layer, 170 represents the electron transport layer, 180 represents the electron injection layer, 190 represents the cathode, where the luminescent layer is a mixed layer of the guest material doped with the host material.

The layers of the organic light-emitting device of the present disclosure can be formed by methods such as vacuum evaporation, sputtering, ion plating, or wet film-forming methods such as spin coating, printing, and printing, without special limitations on the solvents used.

In a preferred embodiment of the present disclosure, an OLED device comprises a hole transport layer, and the hole transport material can be preferably selected from known or unknown materials, particularly preferably from the following structures, but this does not mean that the present disclosure is limited to the following structures:

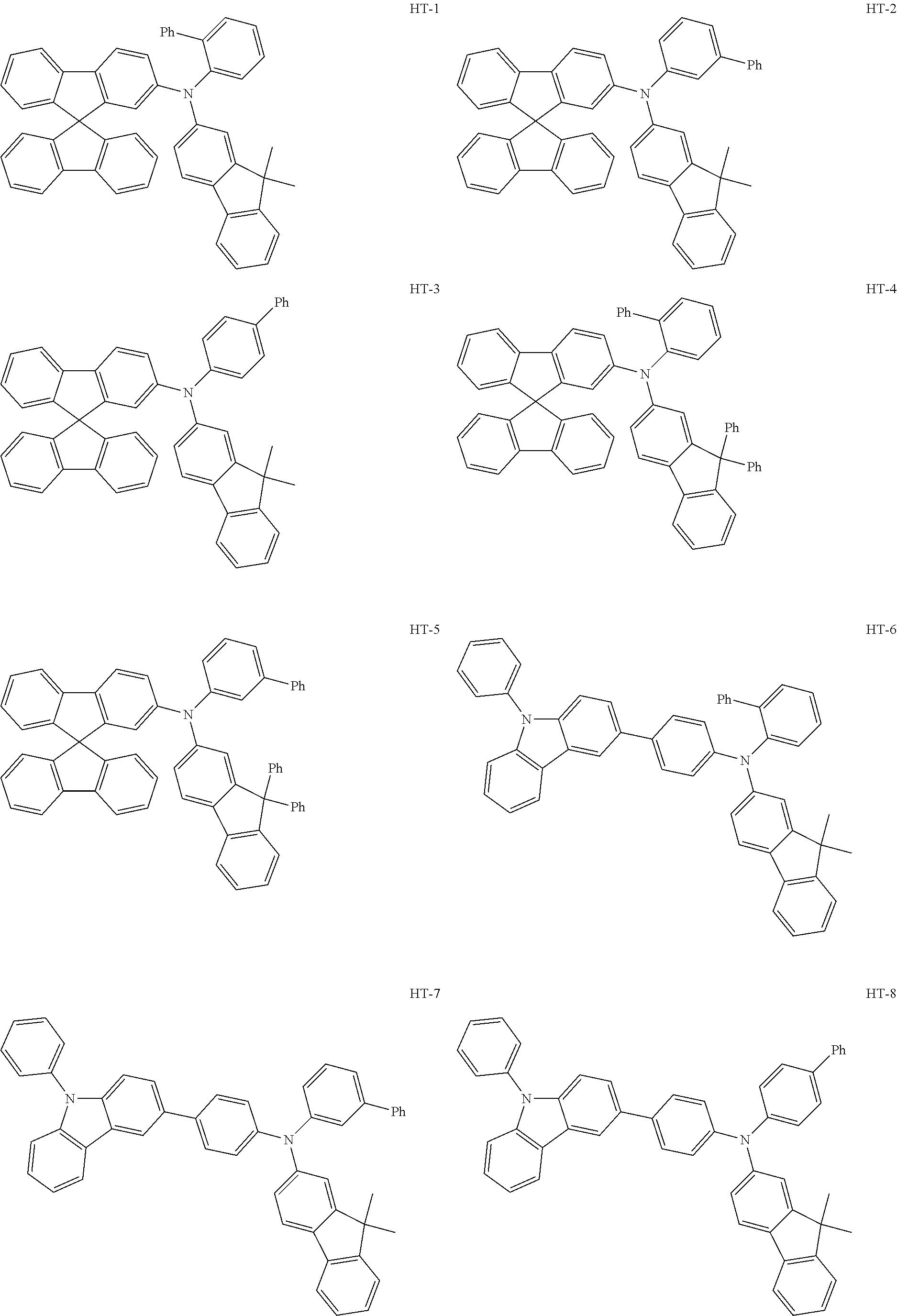

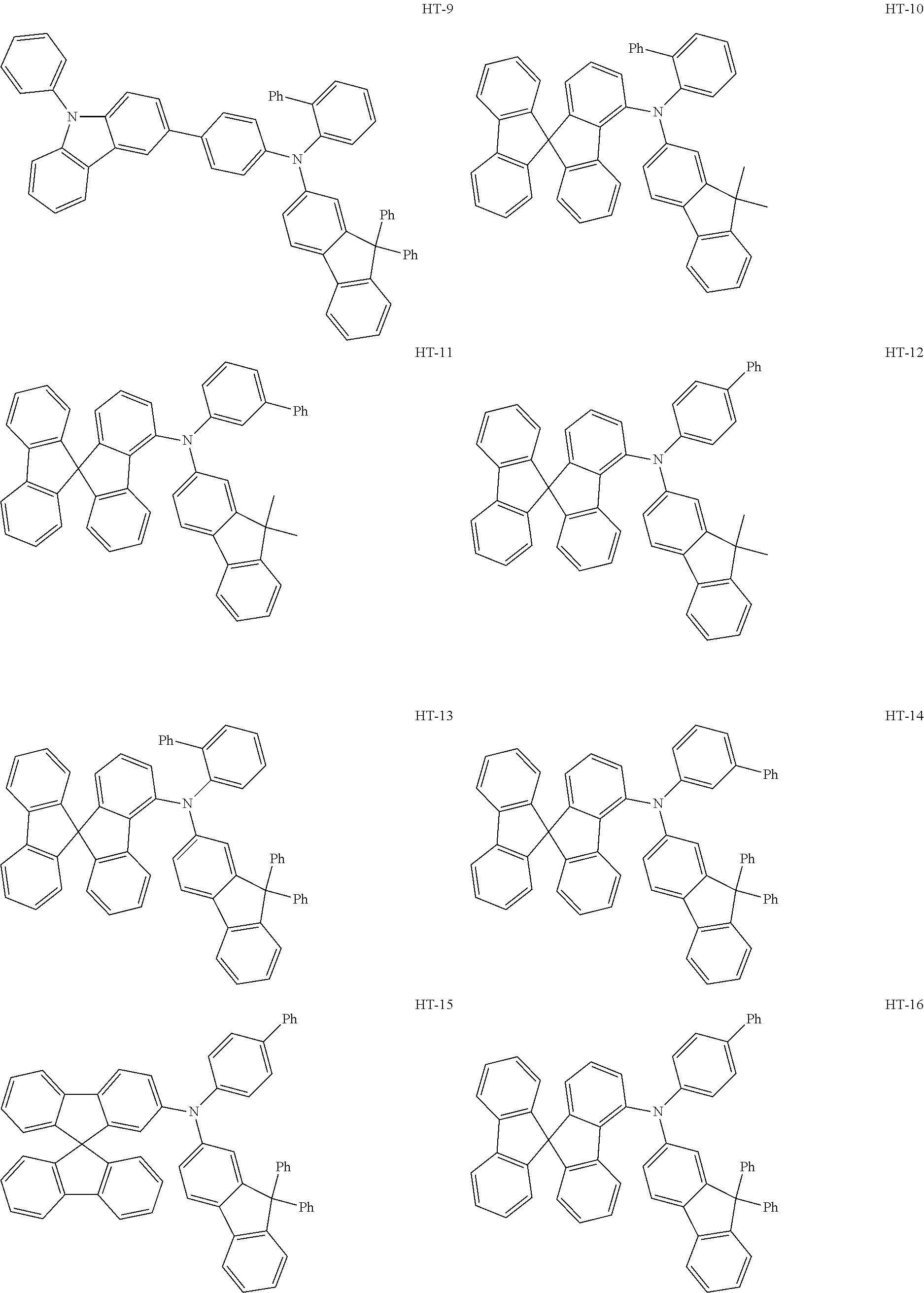

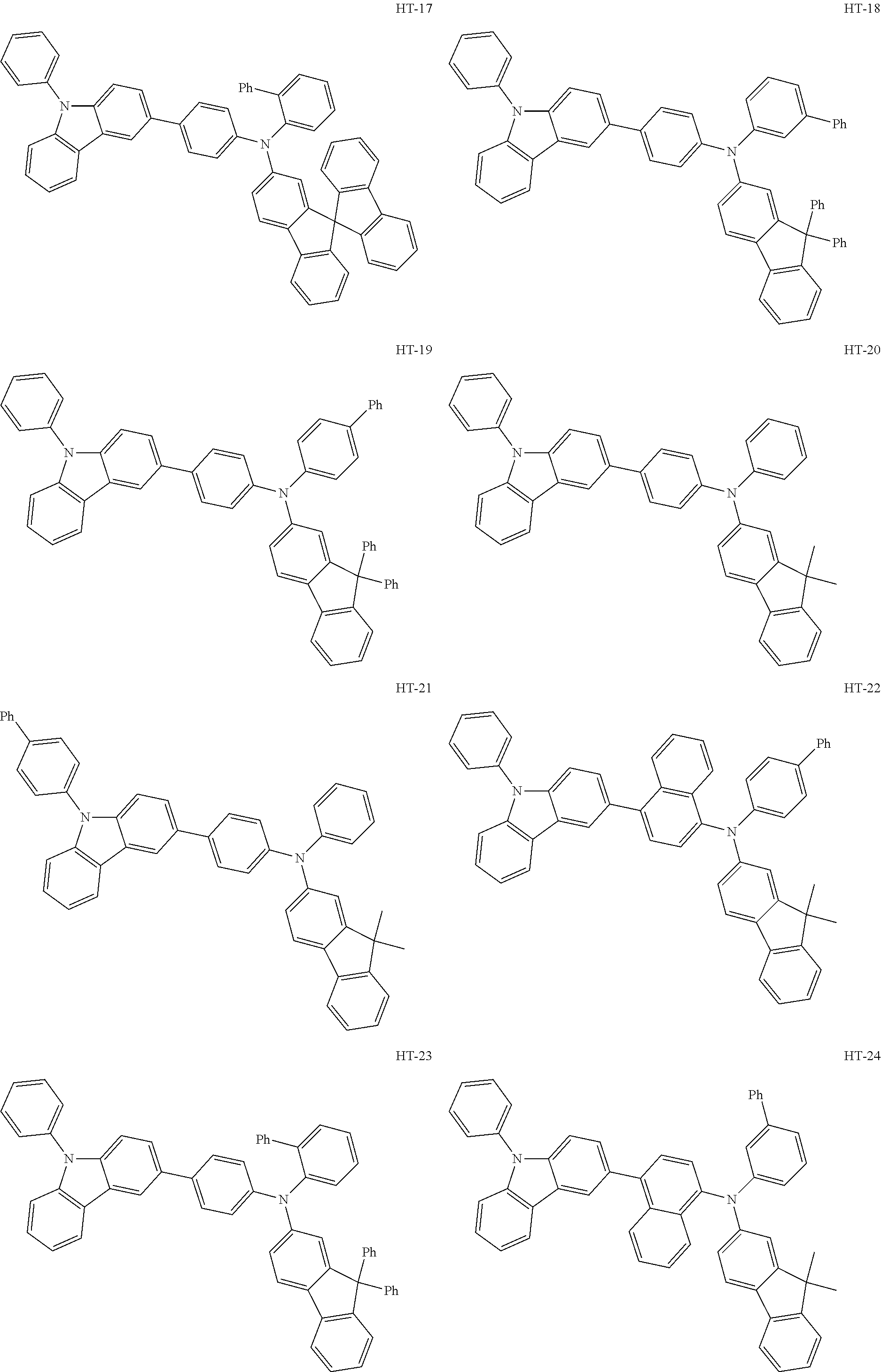

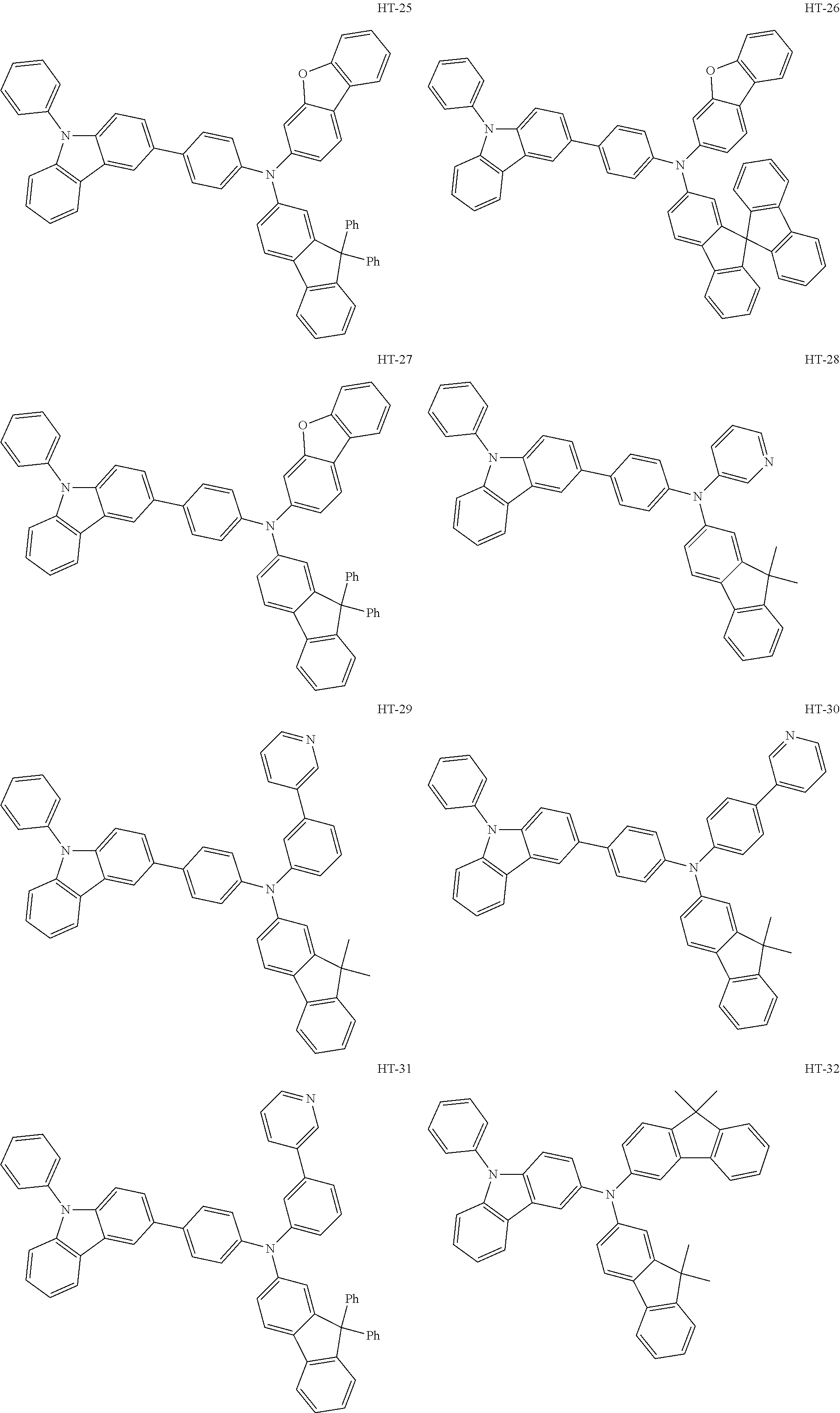

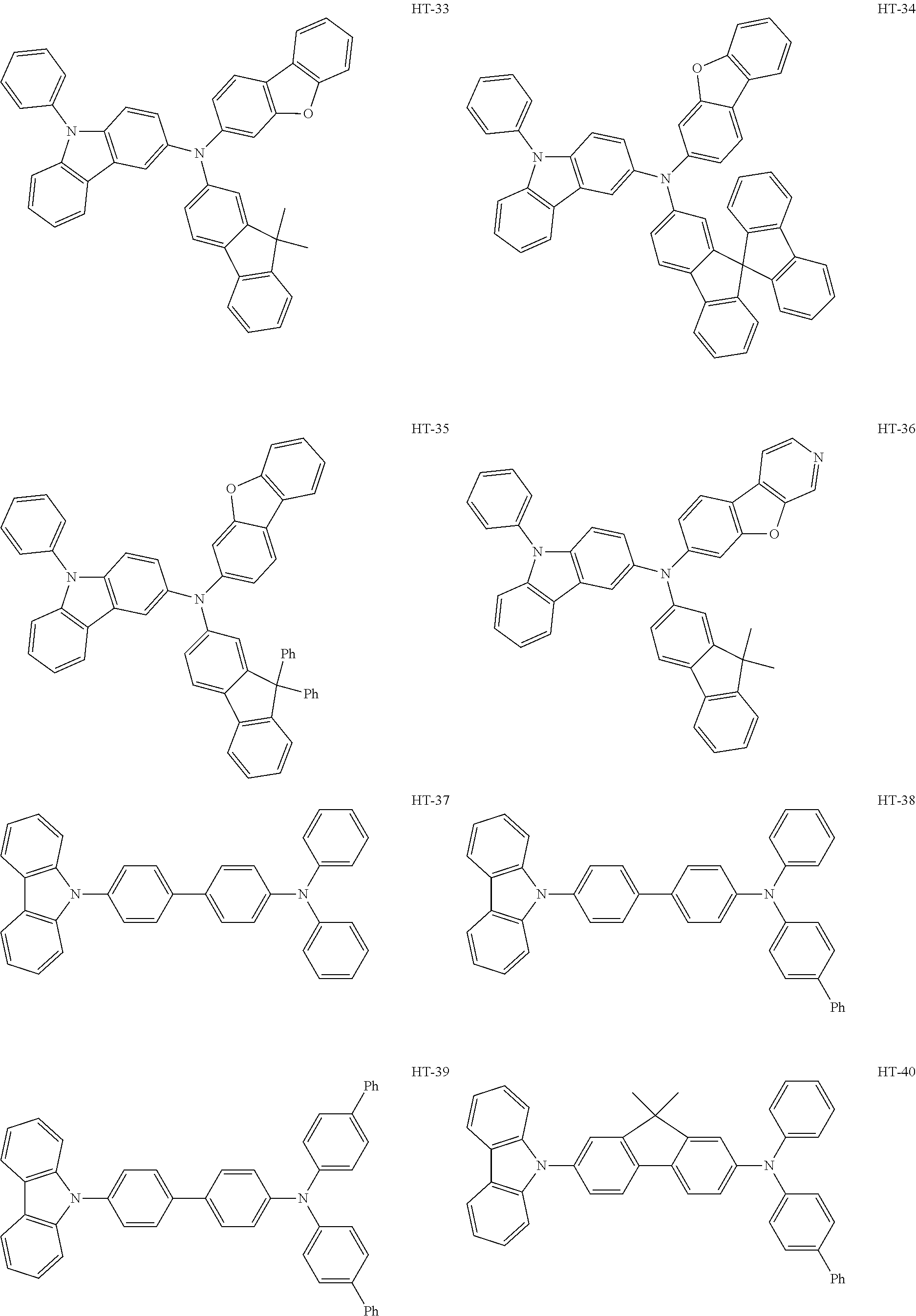

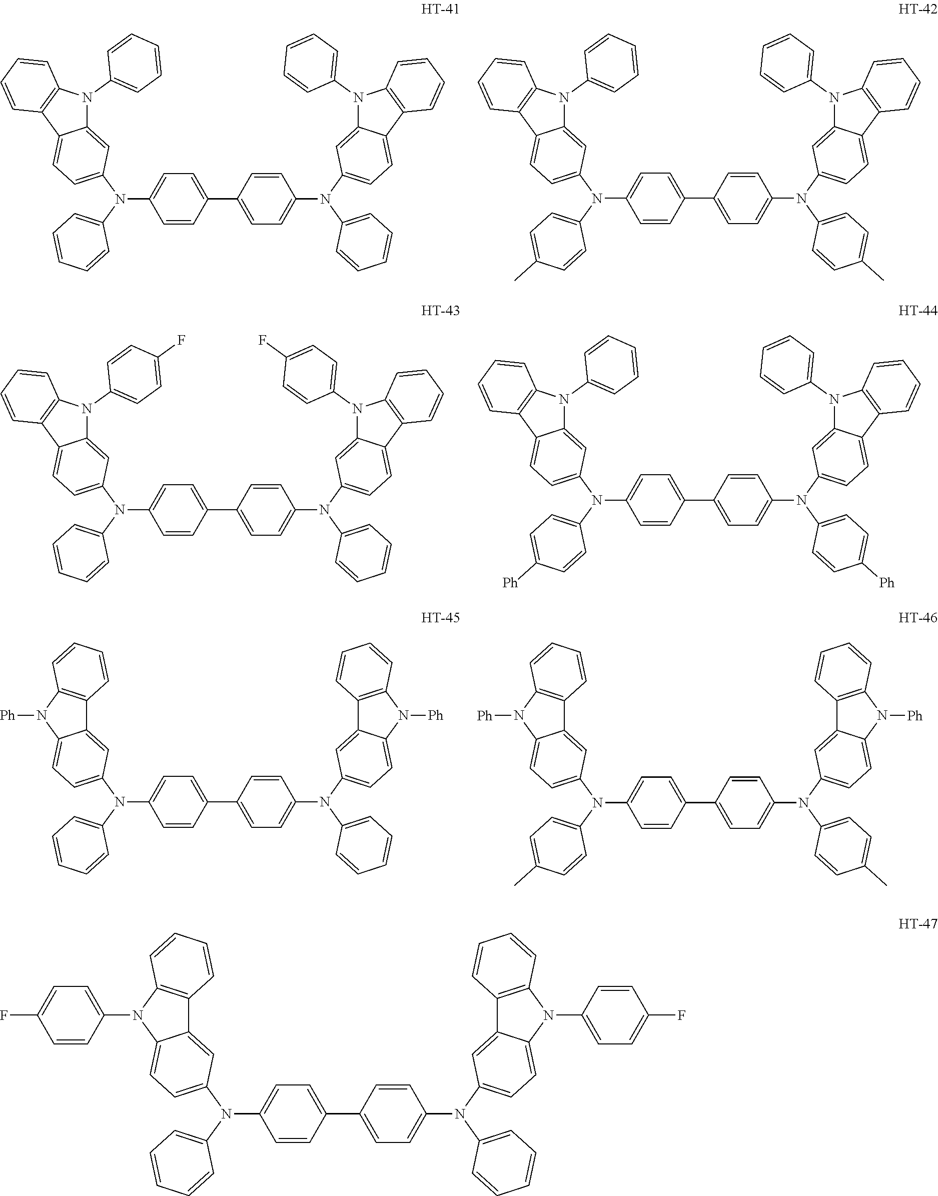

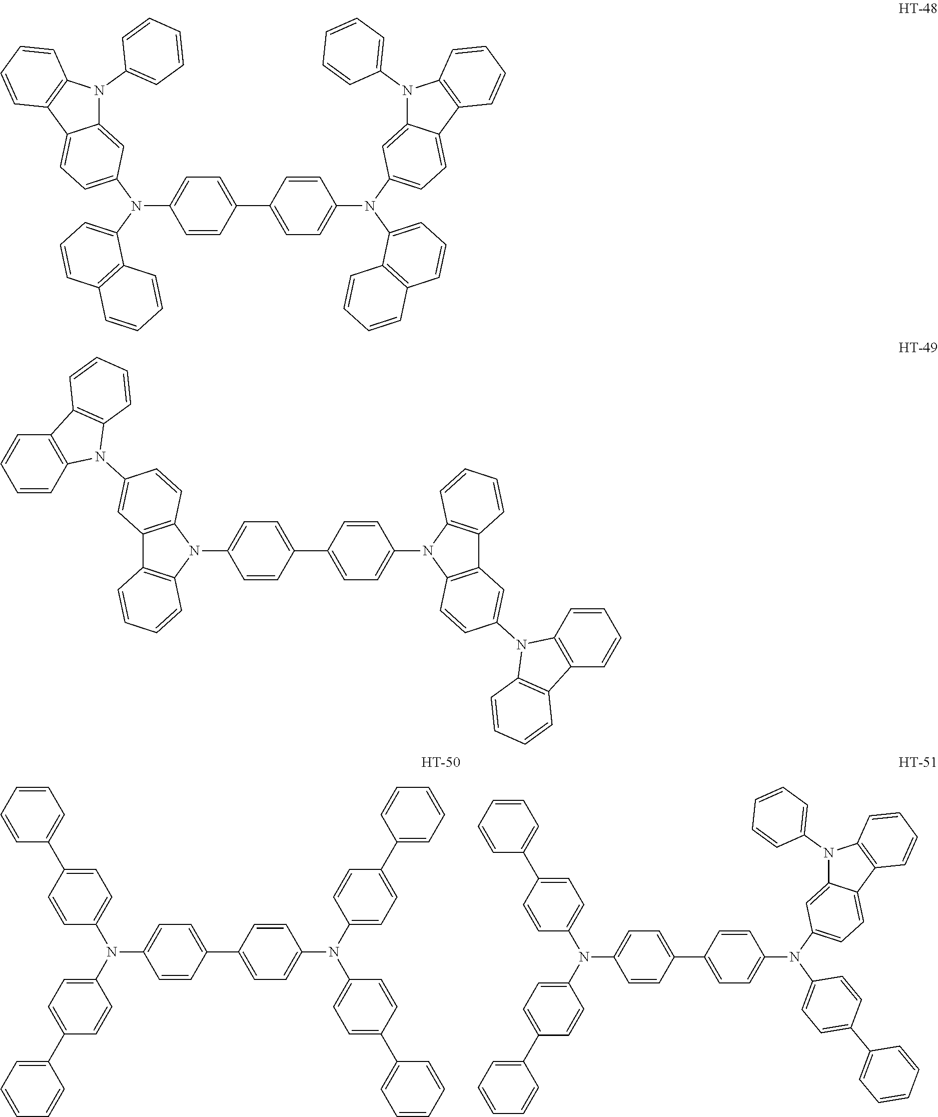

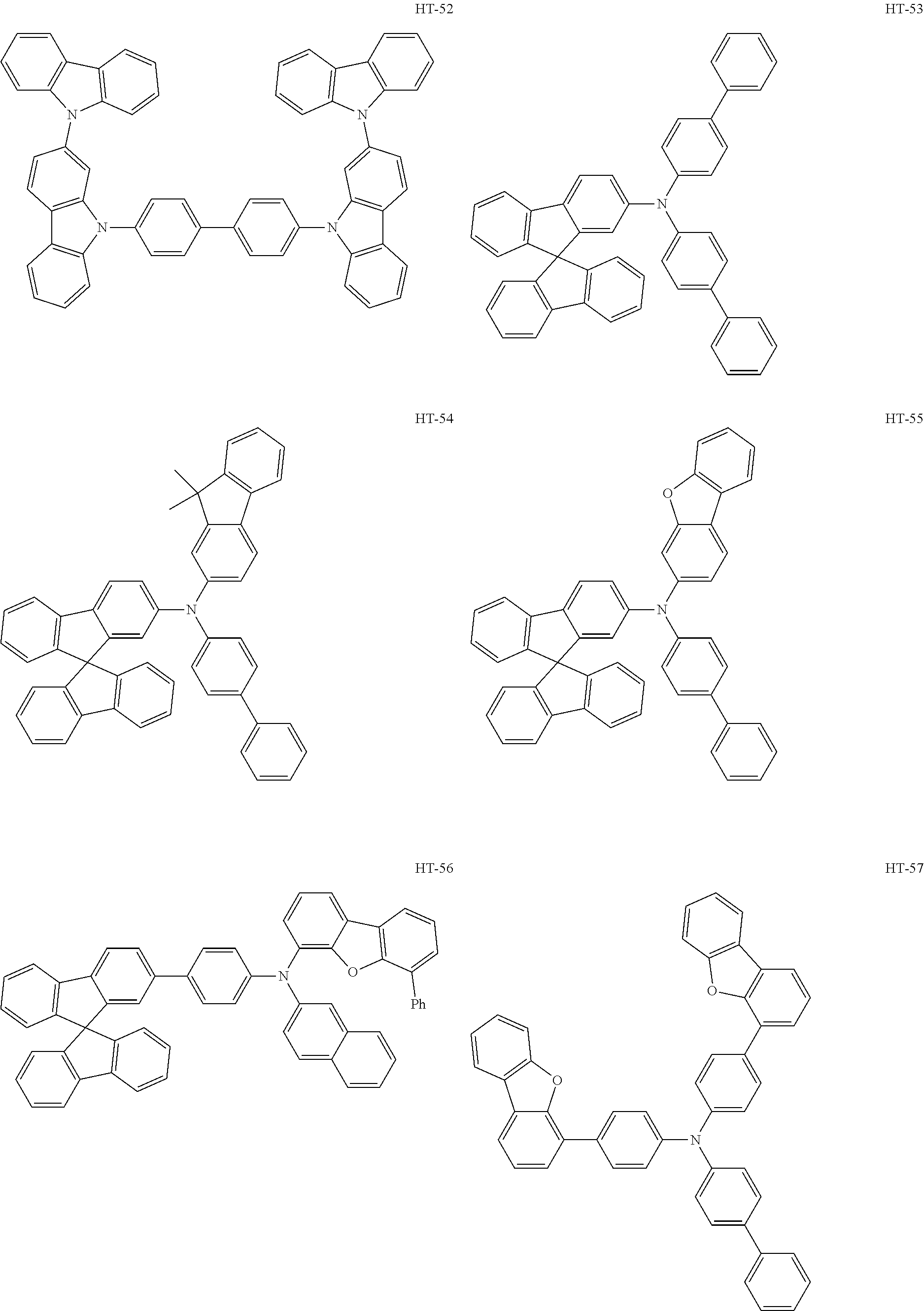

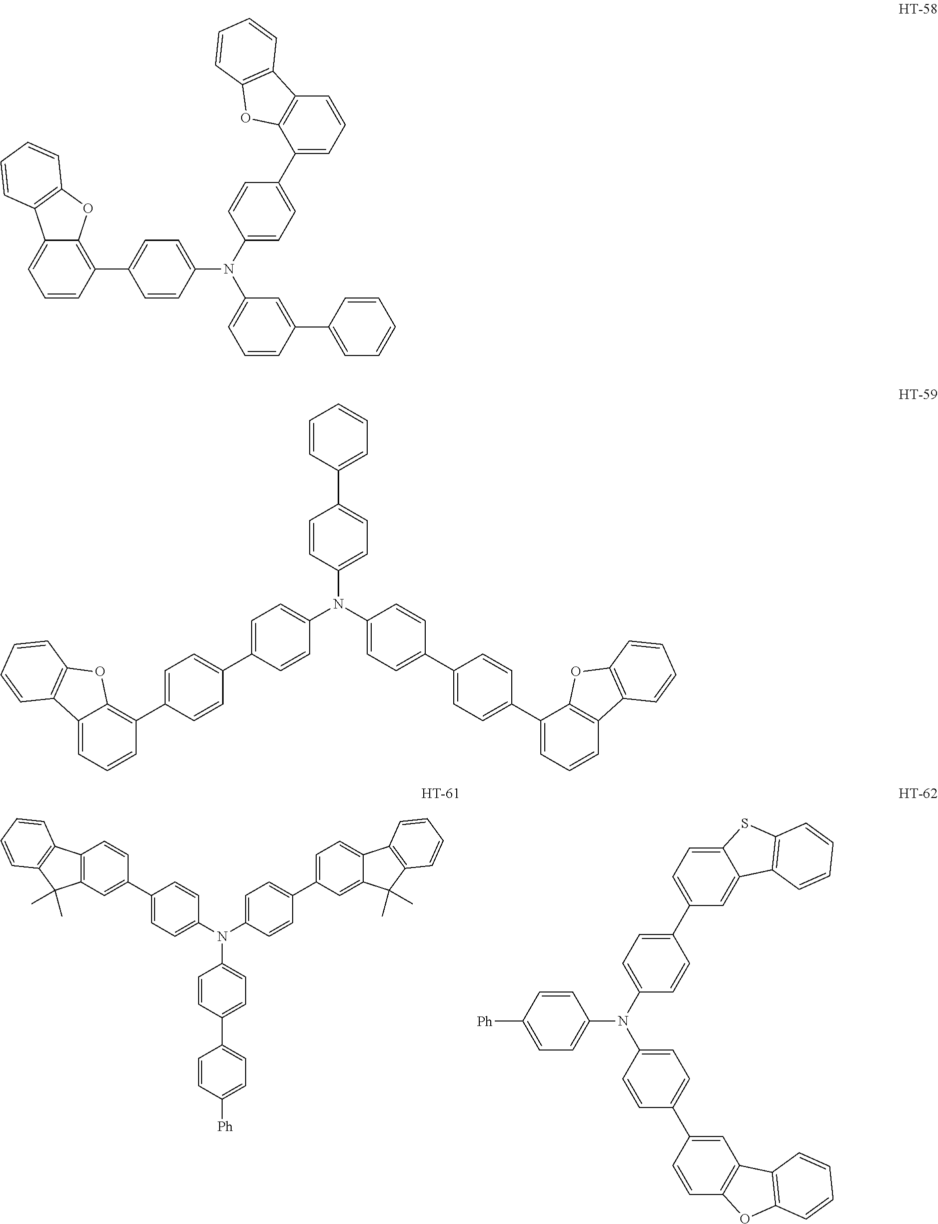 [SMILES: c1ccc(-c2cccc(N(c3ccc(-c4cccc5c4oc4ccccc45)cc3)c3ccc(-c4cccc5c4oc4ccccc45)cc3)c2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21 ; c1ccc(-c2ccc(N(c3ccc(-c4ccc5oc6ccccc6c5c4)cc3)c3ccc(-c4ccc5sc6ccccc6c5c4)cc3)cc2)cc1]

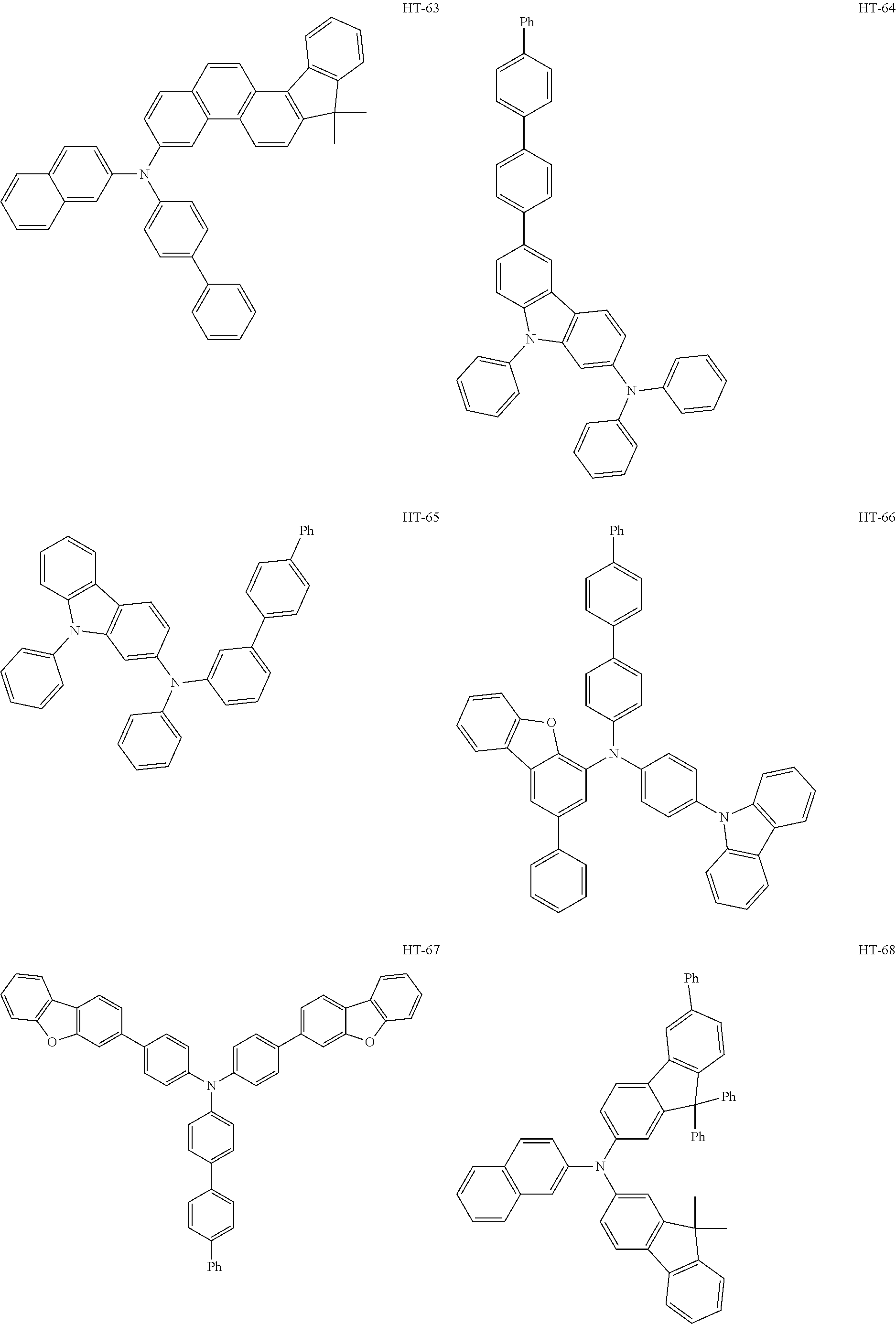

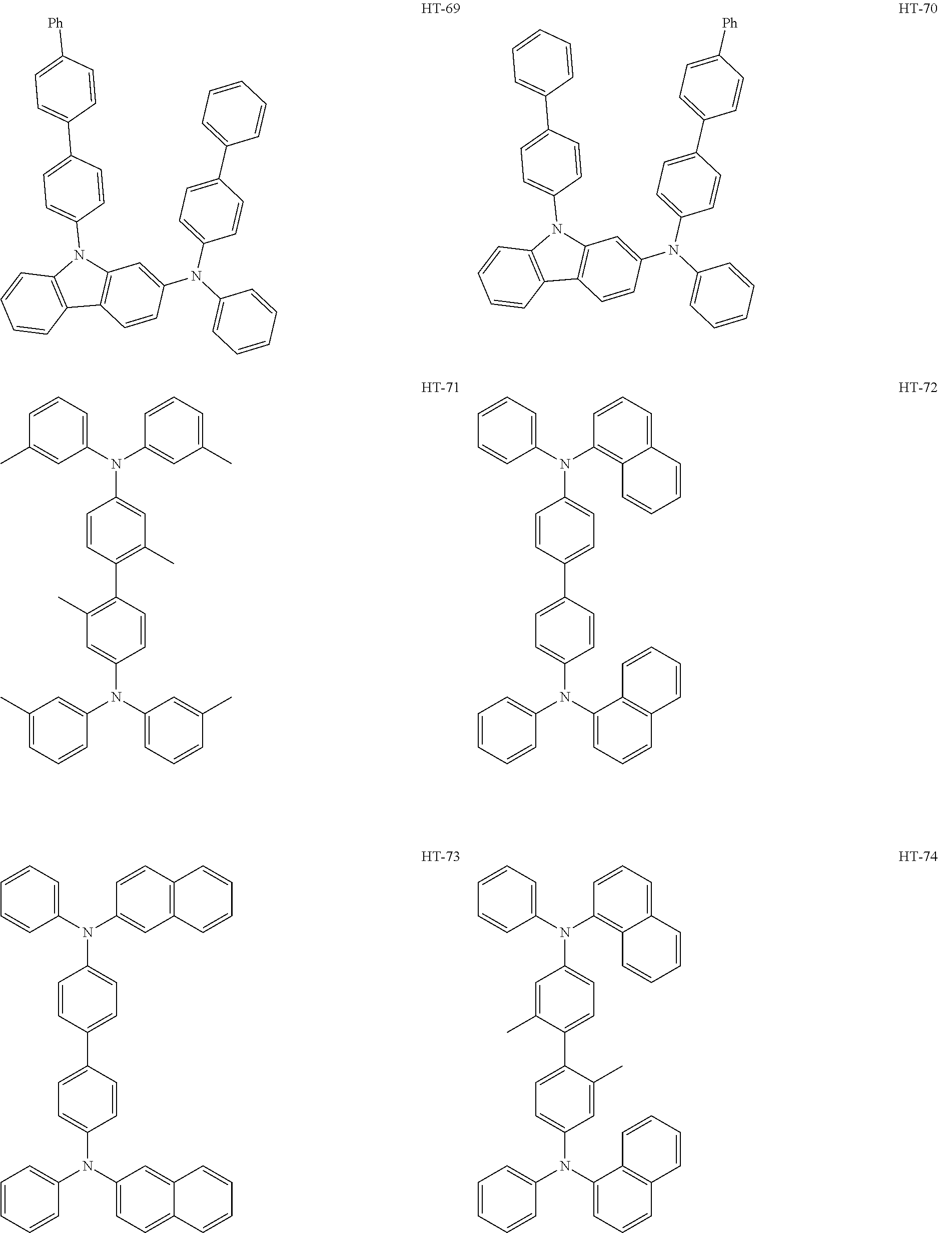

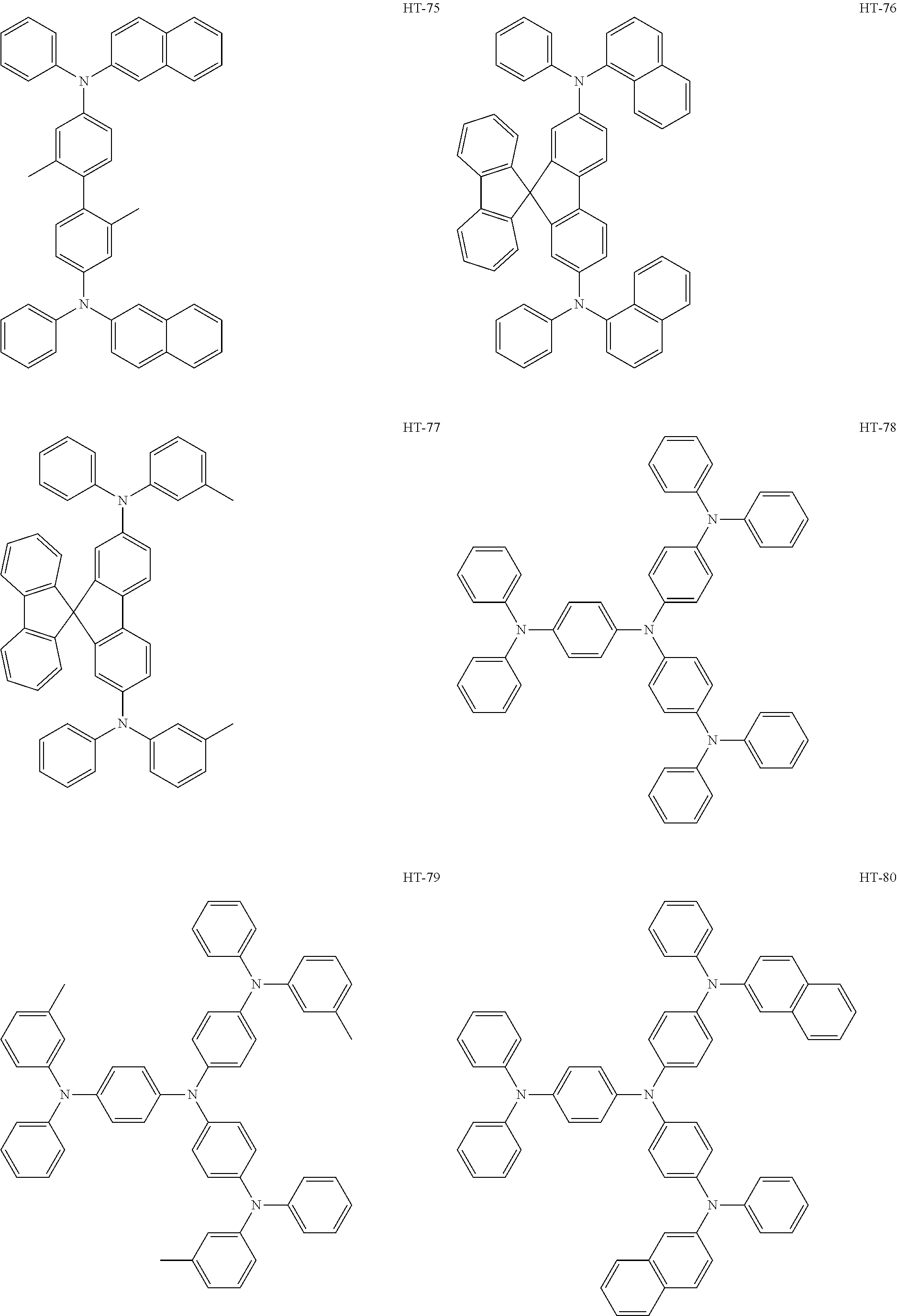

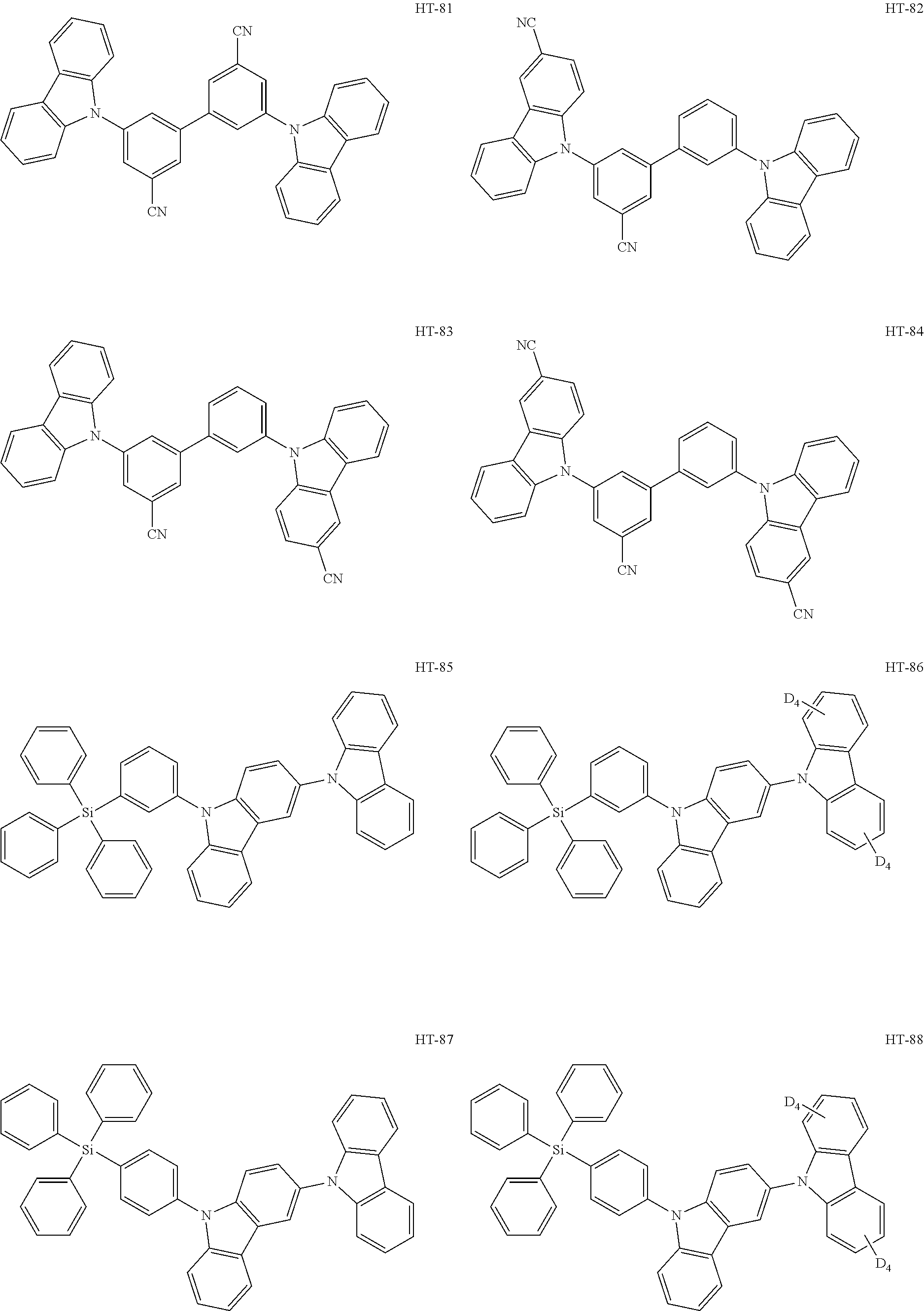

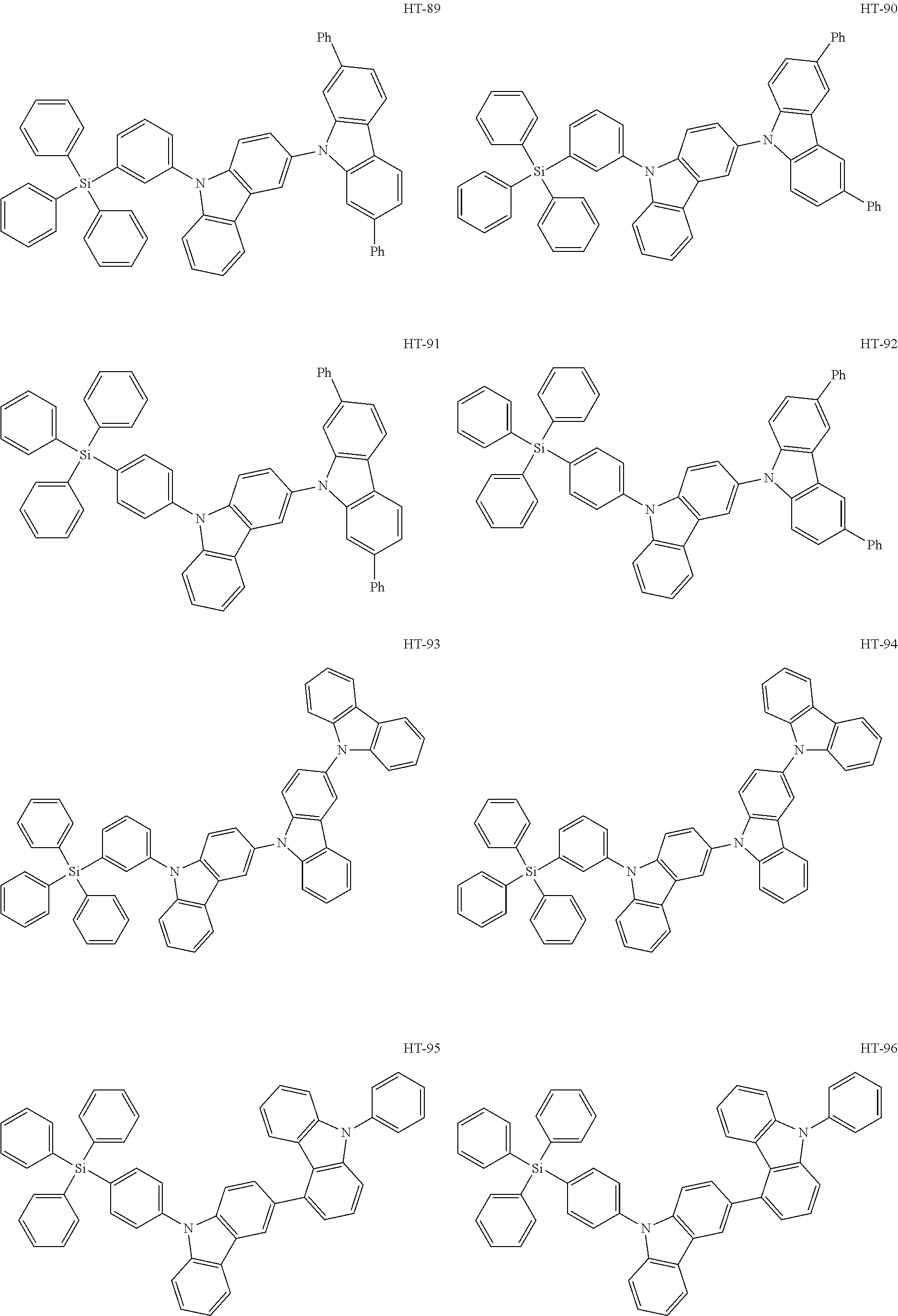

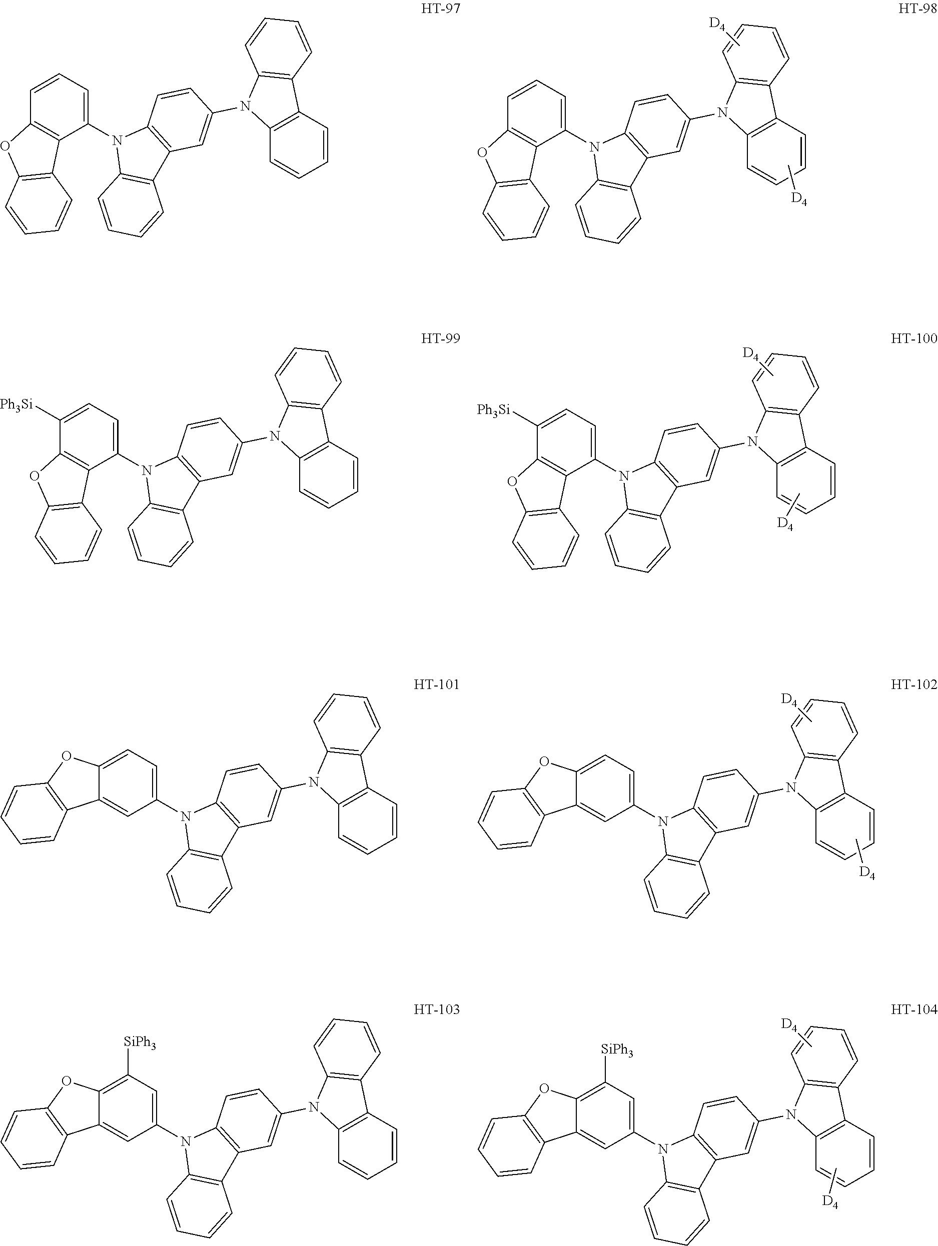

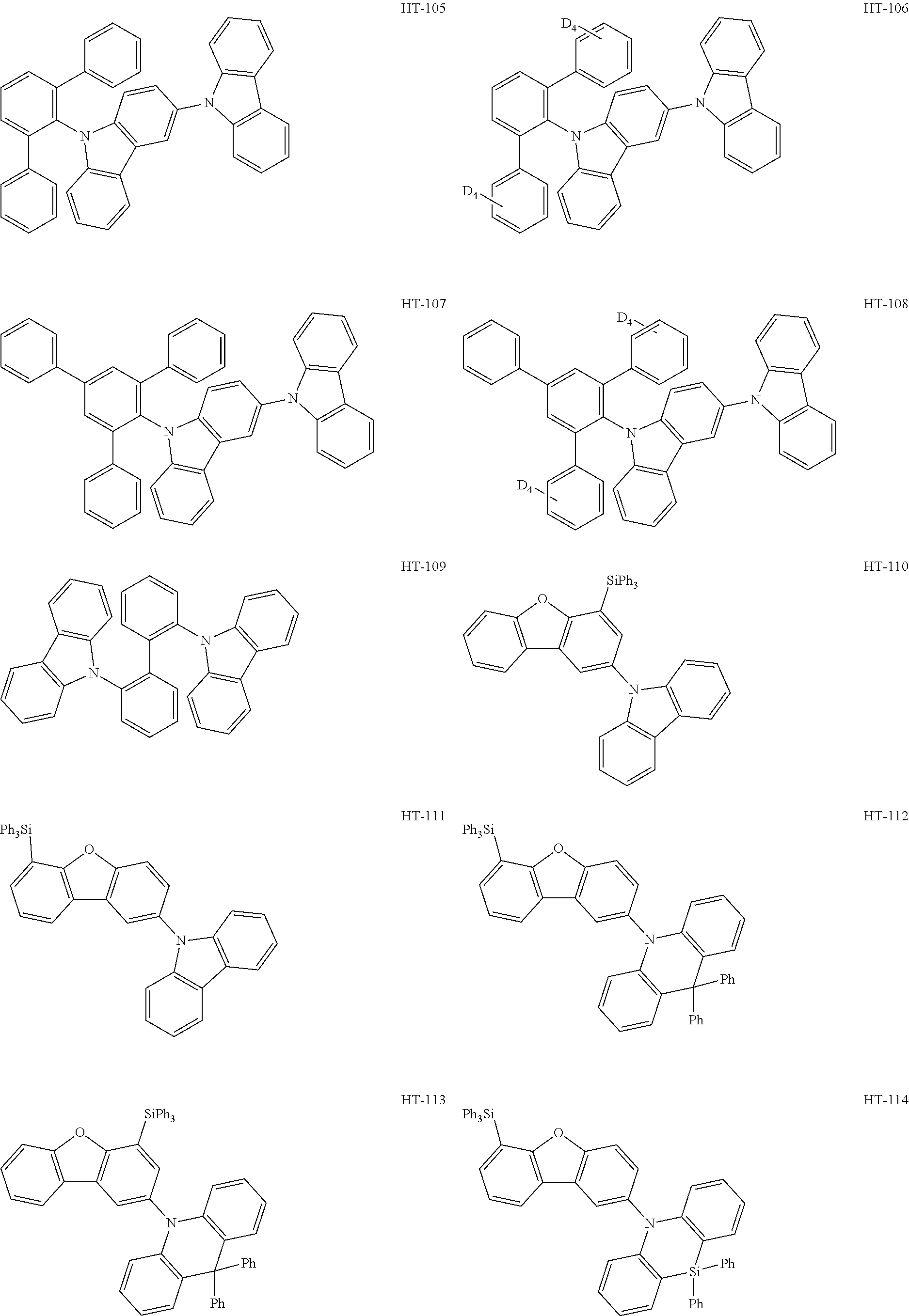

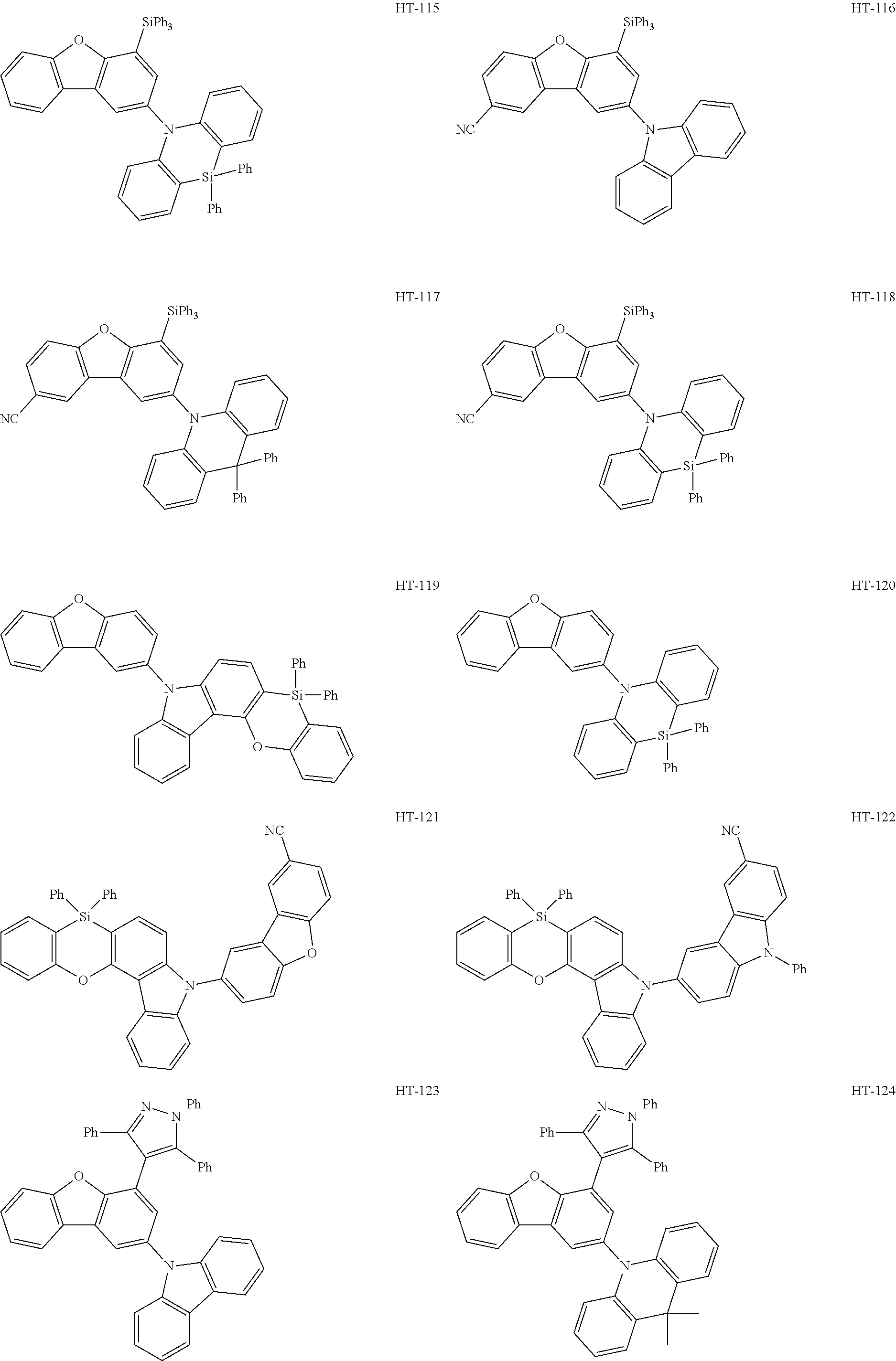

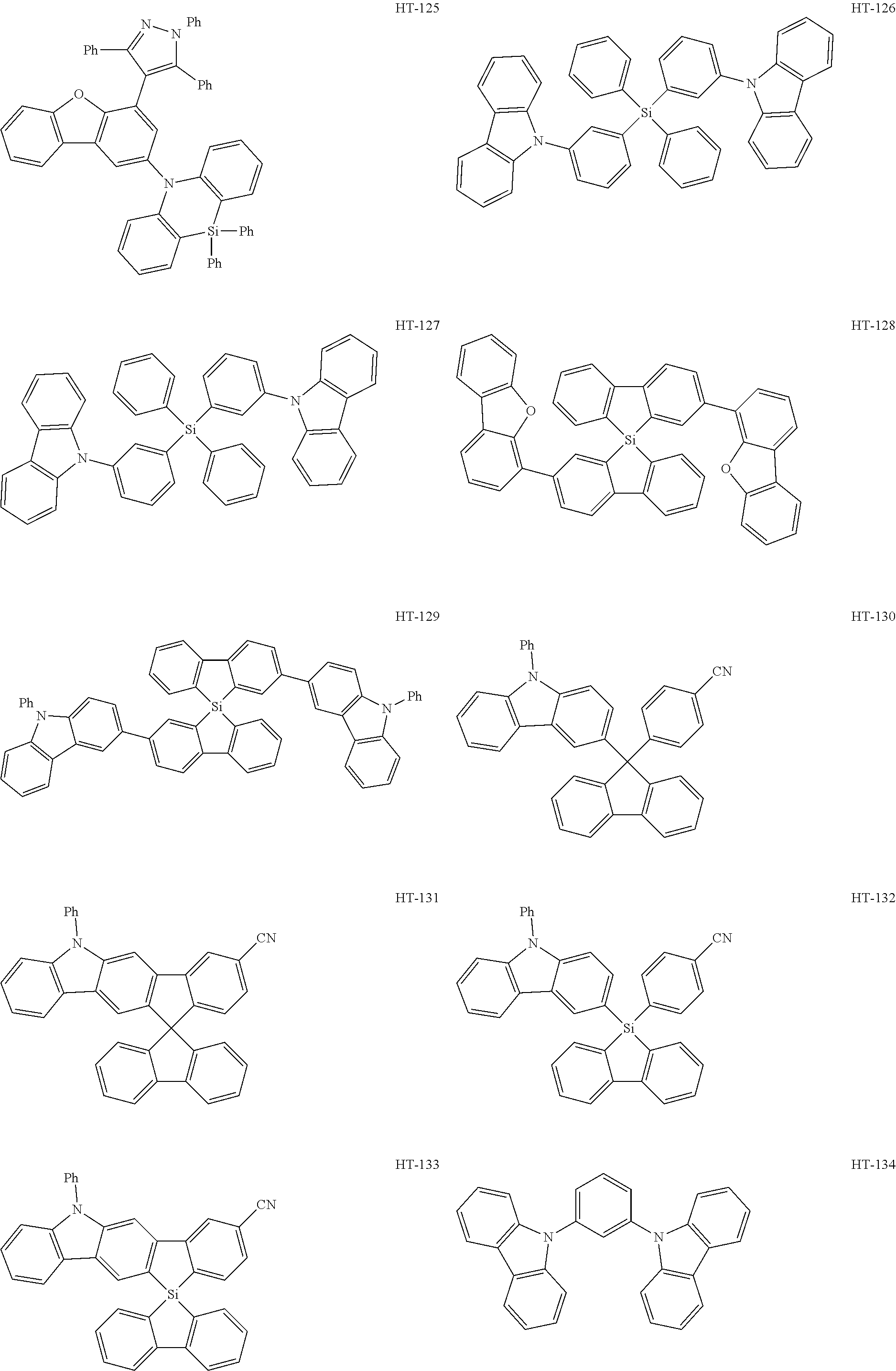

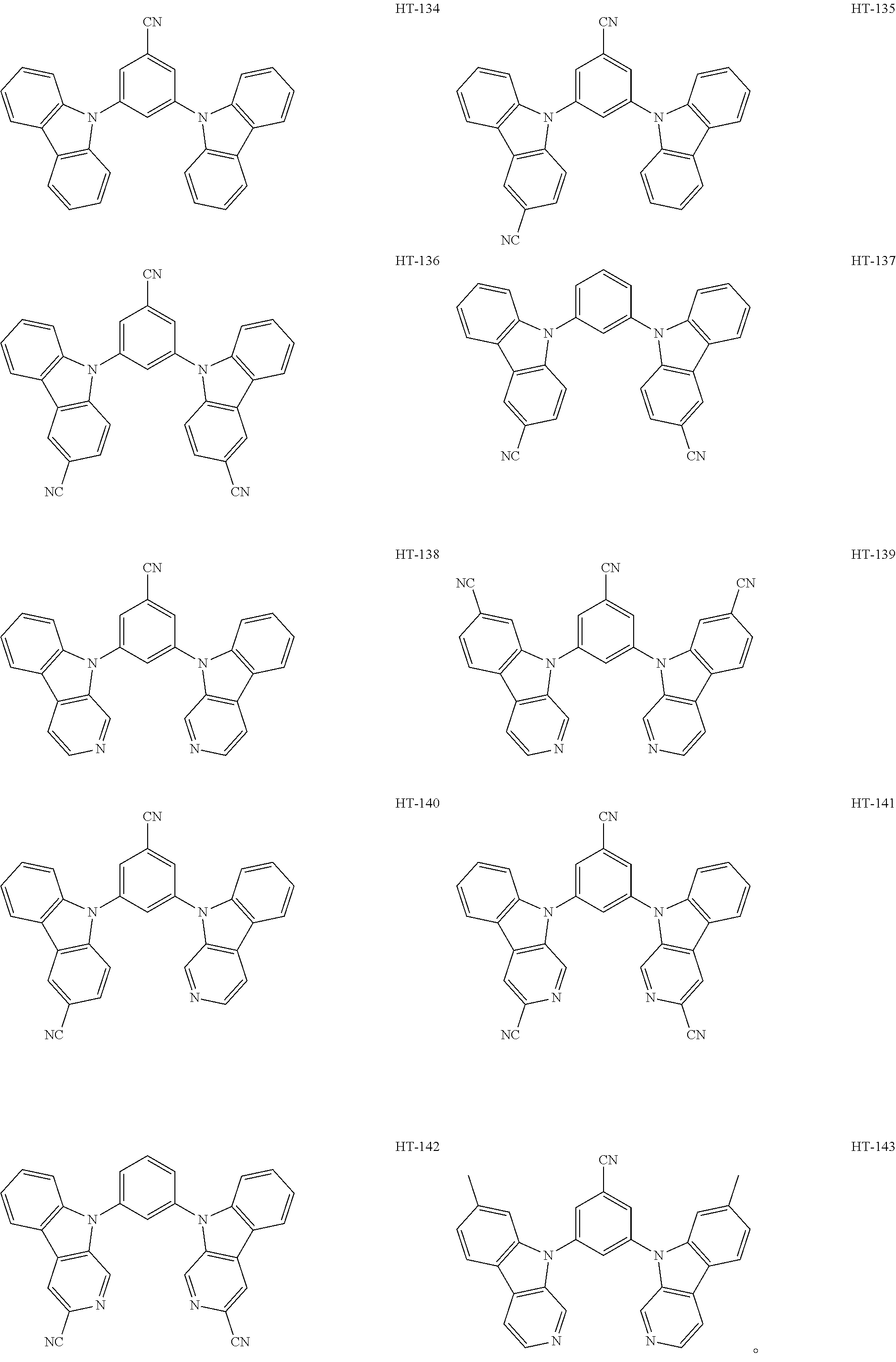

In a preferred embodiment of the present disclosure, the OLED device comprises a hole transport layer comprising one or more p-type dopants. The preferred p-type dopant of the present disclosure is the following structure, but it does not mean that the present disclosure is limited to the following structures:

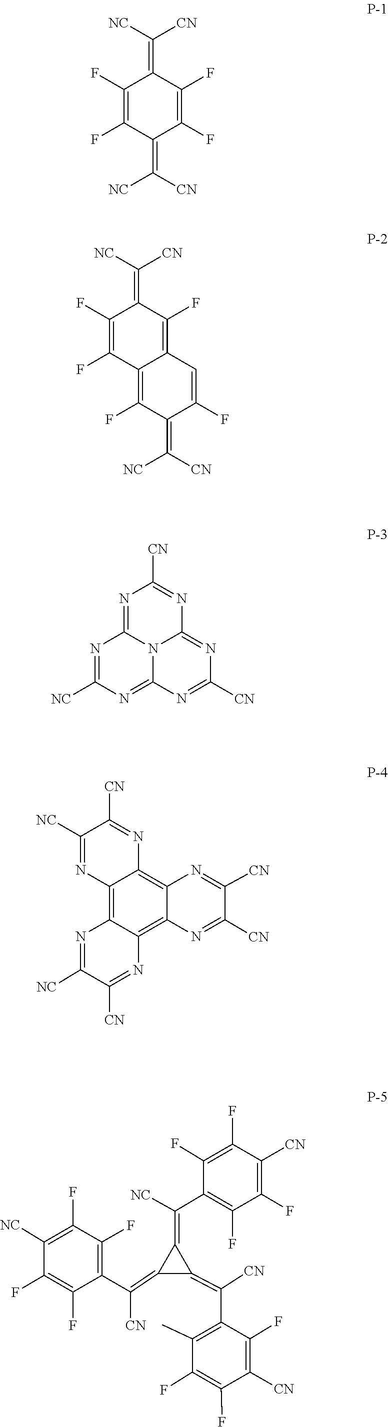 [SMILES: N#CC(C#N)=c1c(F)c(F)c(=C(C#N)C#N)c(F)c1F ; N#CC(C#N)=c1c(F)c(F)c2c(F)c(=C(C#N)C#N)c(F)cc2c1F ; N#CC1=NC2=NC(C#N)=NC3=NC(C#N)=NC(=N1)N23 ; N#Cc1nc2c3nc(C#N)c(C#N)nc3c3nc(C#N)c(C#N)nc3c2nc1C#N ; Cc1c(F)c(F)c(C#N)c(F)c1/C(C#N)=C1/C(=C(/C#N)c2c(F)c(F)c(C#N)c(F)c2F)/C1=C(/C#N)c1c(F)c(F)c(C#N)c(F)c1F]

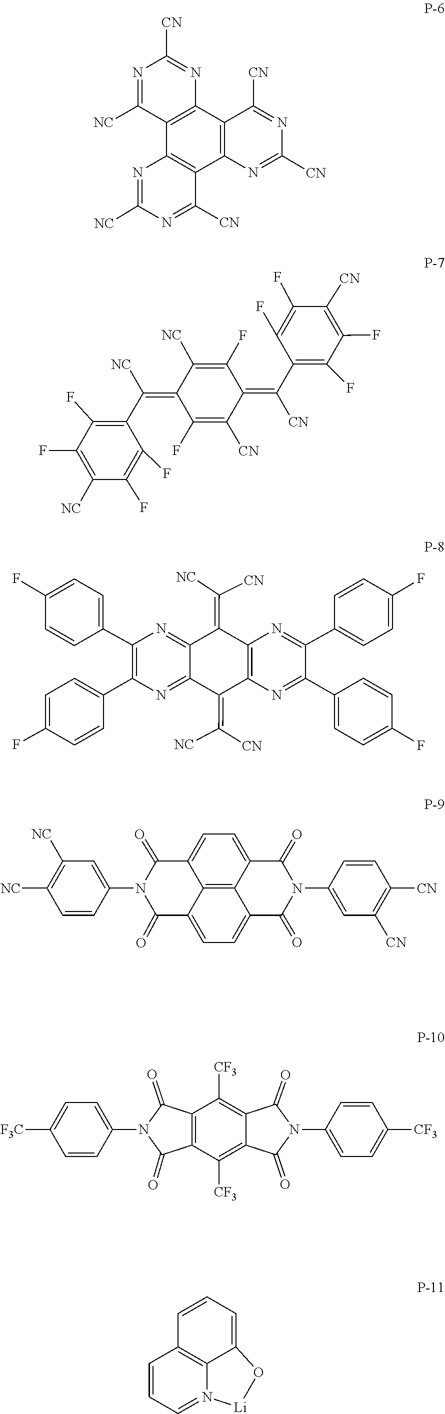

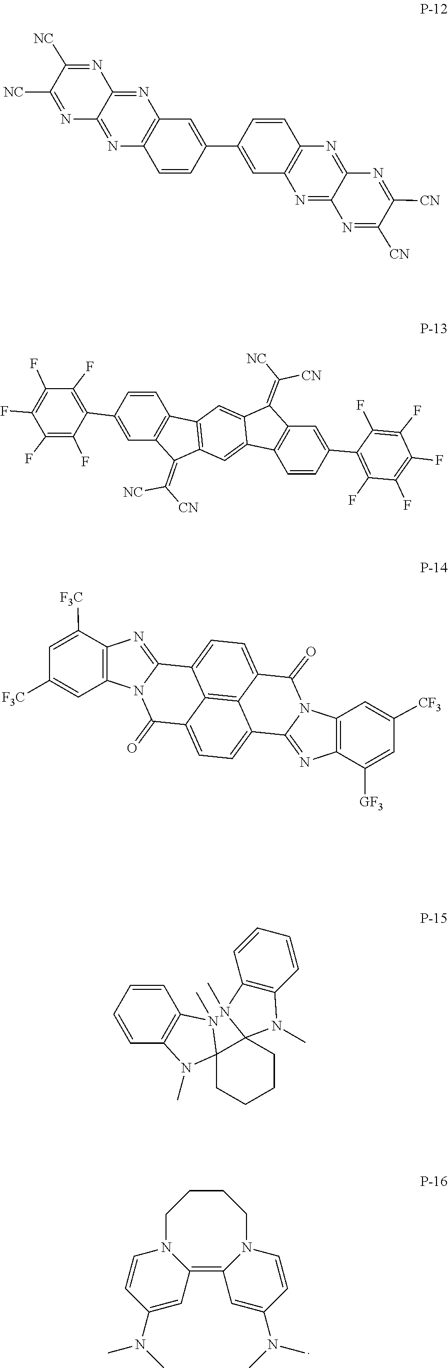

In a preferred embodiment of the present disclosure, the electron transport layer may be selected from at least one of compounds ET-1 to ET-77, but it does not mean that the present disclosure is limited to the following structures:

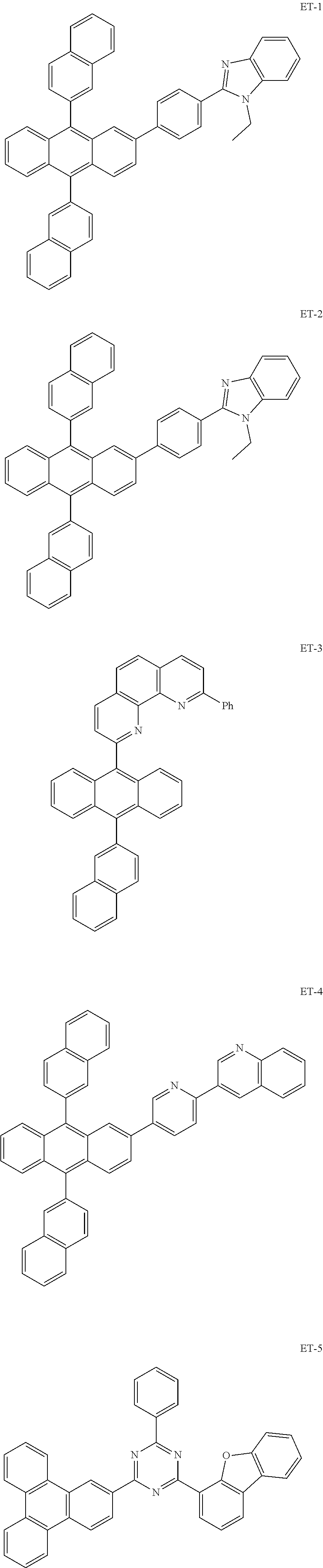 [SMILES: CCn1c(-c2ccc(-c3ccc4c(-c5ccc6ccccc6c5)c5ccccc5c(-c5ccc6ccccc6c5)c4c3)cc2)nc2ccccc21 ; CCn1c(-c2ccc(-c3ccc4c(-c5ccc6ccccc6c5)c5ccccc5c(-c5ccc6ccccc6c5)c4c3)cc2)nc2ccccc21 ; c1ccc(-c2ccc3ccc4ccc(-c5c6ccccc6c(-c6ccc7ccccc7c6)c6ccccc56)nc4c3n2)cc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-c2nc(-c3ccc4c5ccccc5c5ccccc5c4c3)nc(-c3cccc4c3oc3ccccc34)n2)cc1]

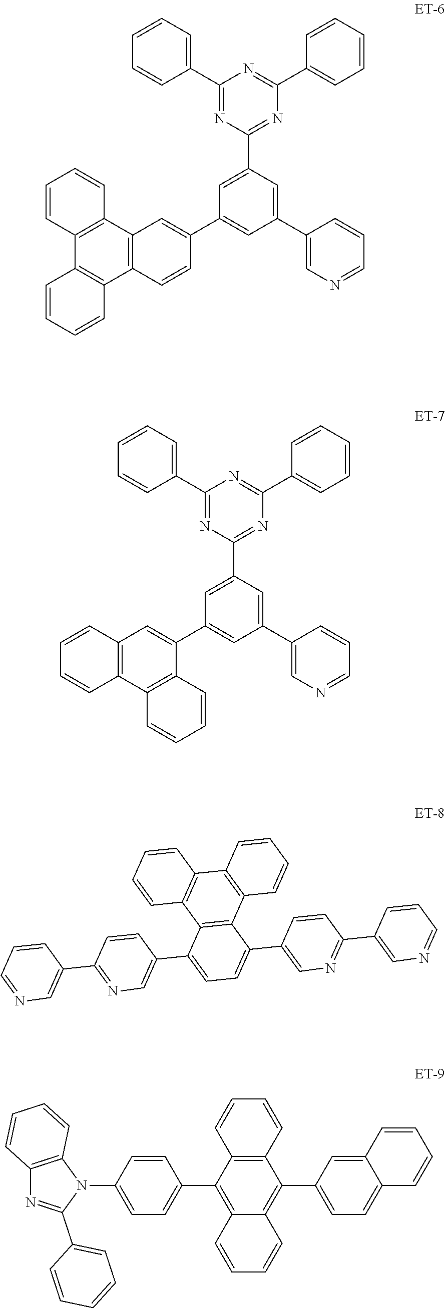 [SMILES: c1ccc(-c2nc(-c3ccccc3)nc(-c3cc(-c4cccnc4)cc(-c4ccc5c6ccccc6c6ccccc6c5c4)c3)n2)cc1 ; c1ccc(-c2nc(-c3ccccc3)nc(-c3cc(-c4cccnc4)cc(-c4cc5ccccc5c5ccccc45)c3)n2)cc1 ; c1cncc(-c2ccc(-c3ccc(-c4ccc(-c5cccnc5)nc4)c4c5ccccc5c5ccccc5c34)cn2)c1 ; c1ccc(-c2nc3ccccc3n2-c2ccc(-c3c4ccccc4c(-c4ccc5ccccc5c4)c4ccccc34)cc2)cc1]

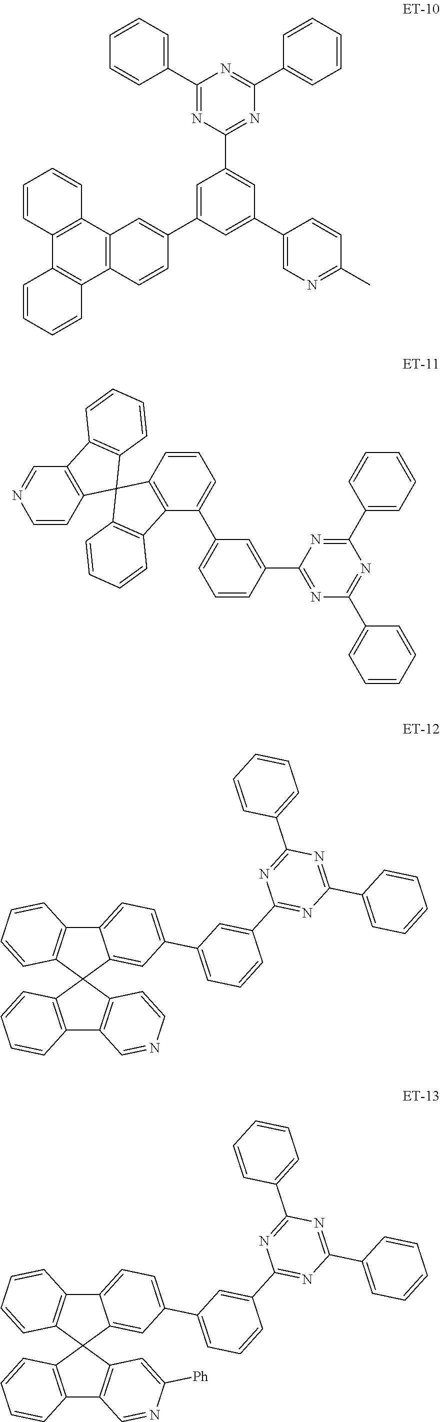 [SMILES: Cc1ccc(-c2cc(-c3ccc4c5ccccc5c5ccccc5c4c3)cc(-c3nc(-c4ccccc4)nc(-c4ccccc4)n3)c2)cn1 ; c1ccc(-c2nc(-c3ccccc3)nc(-c3cccc(-c4cccc5c4-c4ccccc4C54c5ccccc5-c5cnccc54)c3)n2)cc1 ; c1ccc(-c2nc(-c3ccccc3)nc(-c3cccc(-c4ccc5c(c4)C4(c6ccccc6-c6cnccc64)c4ccccc4-5)c3)n2)cc1 ; c1ccc(-c2cc3c(cn2)-c2ccccc2C32c3ccccc3-c3ccc(-c4cccc(-c5nc(-c6ccccc6)nc(-c6ccccc6)n5)c4)cc32)cc1]

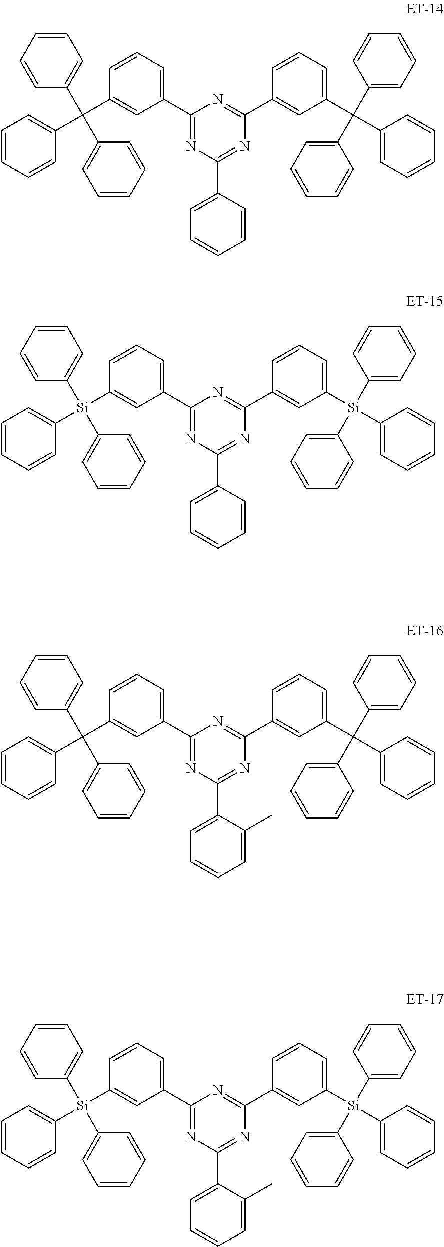 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)nc(-c2cccc([Si](c3ccccc3)(c3ccccc3)c3ccccc3)c2)n1]

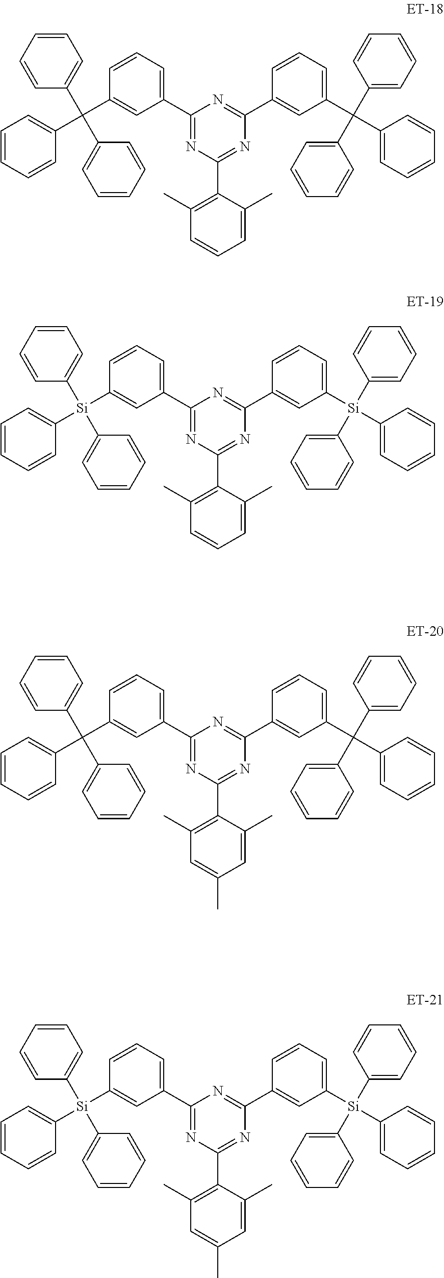 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

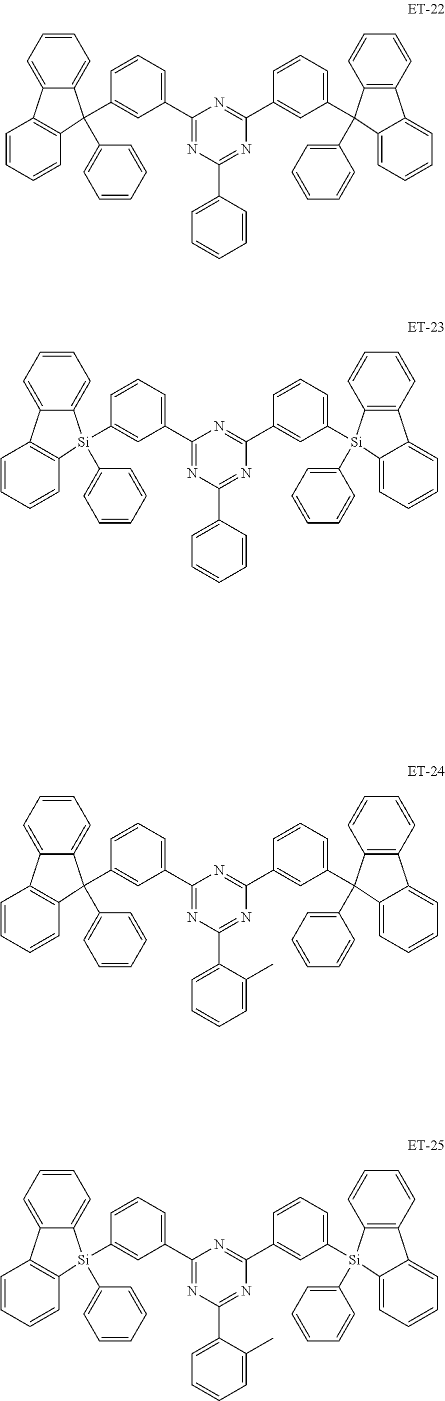 [SMILES: c1ccc(-c2nc(-c3cccc(C4(c5ccccc5)c5ccccc5-c5ccccc54)c3)nc(-c3cccc(C4(c5ccccc5)c5ccccc5-c5ccccc54)c3)n2)cc1 ; c1ccc(-c2nc(-c3cccc([Si]4(c5ccccc5)c5ccccc5-c5ccccc54)c3)nc(-c3cccc([Si]4(c5ccccc5)c5ccccc5-c5ccccc54)c3)n2)cc1 ; Cc1ccccc1-c1nc(-c2cccc(C3(c4ccccc4)c4ccccc4-c4ccccc43)c2)nc(-c2cccc(C3(c4ccccc4)c4ccccc4-c4ccccc43)c2)n1 ; Cc1ccccc1-c1nc(-c2cccc([Si]3(c4ccccc4)c4ccccc4-c4ccccc43)c2)nc(-c2cccc([Si]3(c4ccccc4)c4ccccc4-c4ccccc43)c2)n1]

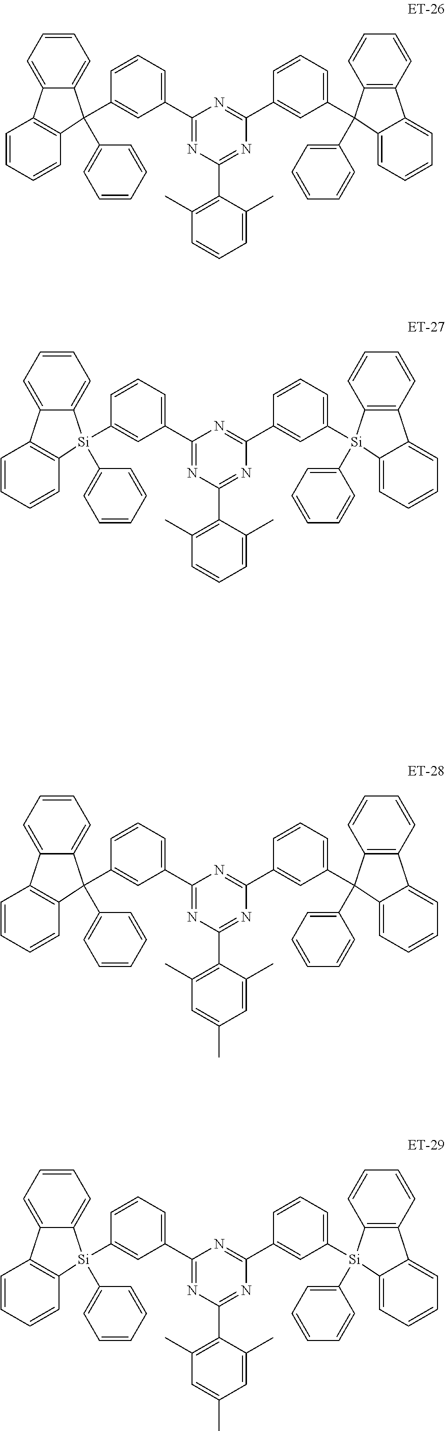 [SMILES: Cc1cccc(C)c1-c1nc(-c2cccc(C3(c4ccccc4)c4ccccc4-c4ccccc43)c2)nc(-c2cccc(C3(c4ccccc4)c4ccccc4-c4ccccc43)c2)n1 ; Cc1cccc(C)c1-c1nc(-c2cccc([Si]3(c4ccccc4)c4ccccc4-c4ccccc43)c2)nc(-c2cccc([Si]3(c4ccccc4)c4ccccc4-c4ccccc43)c2)n1 ; Cc1cc(C)c(-c2nc(-c3cccc(C4(c5ccccc5)c5ccccc5-c5ccccc54)c3)nc(-c3cccc(C4(c5ccccc5)c5ccccc5-c5ccccc54)c3)n2)c(C)c1 ; Cc1cc(C)c(-c2nc(-c3cccc([Si]4(c5ccccc5)c5ccccc5-c5ccccc54)c3)nc(-c3cccc([Si]4(c5ccccc5)c5ccccc5-c5ccccc54)c3)n2)c(C)c1]

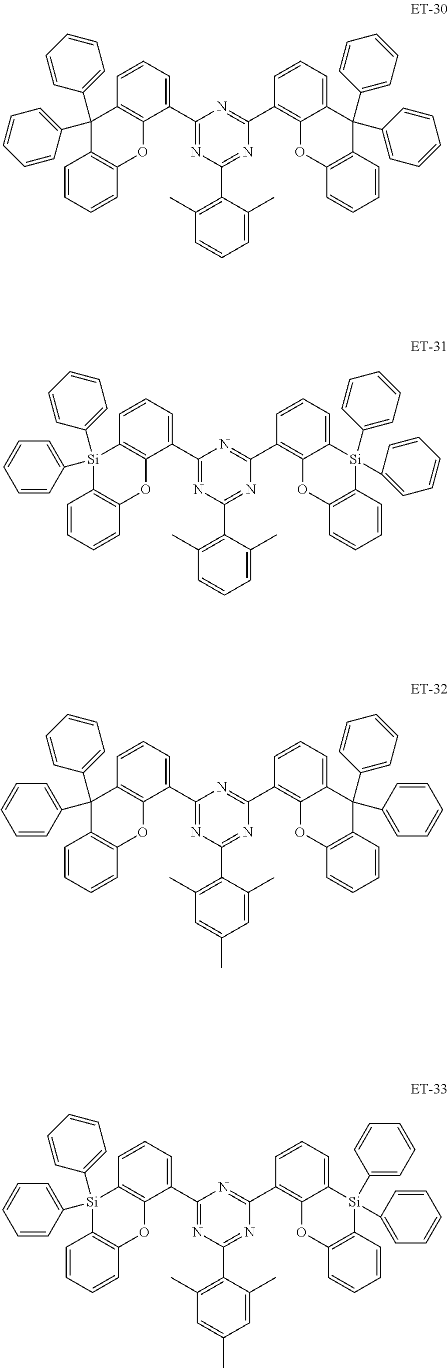 [SMILES: Cc1cccc(C)c1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)nc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1]

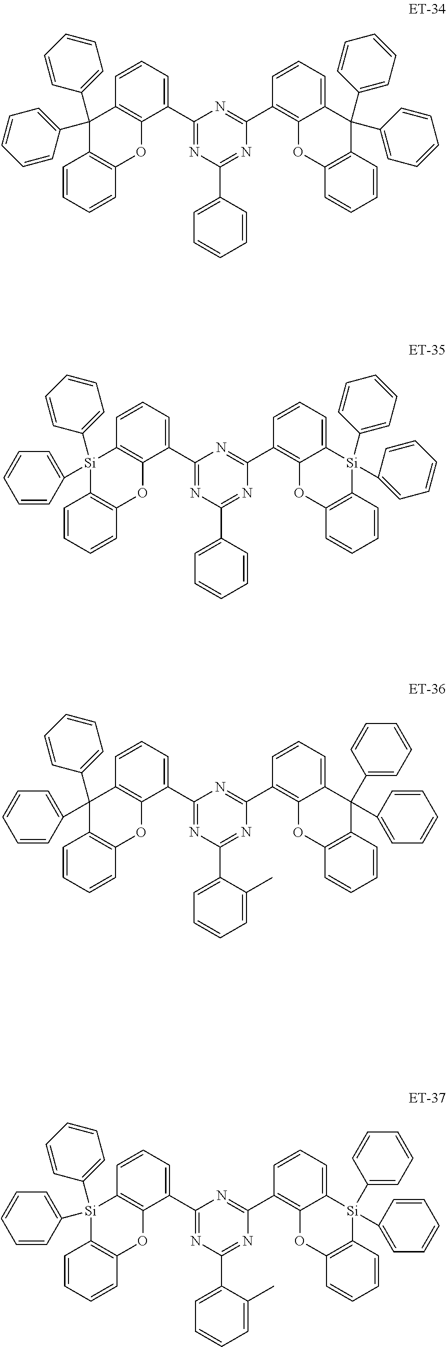 [SMILES: c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

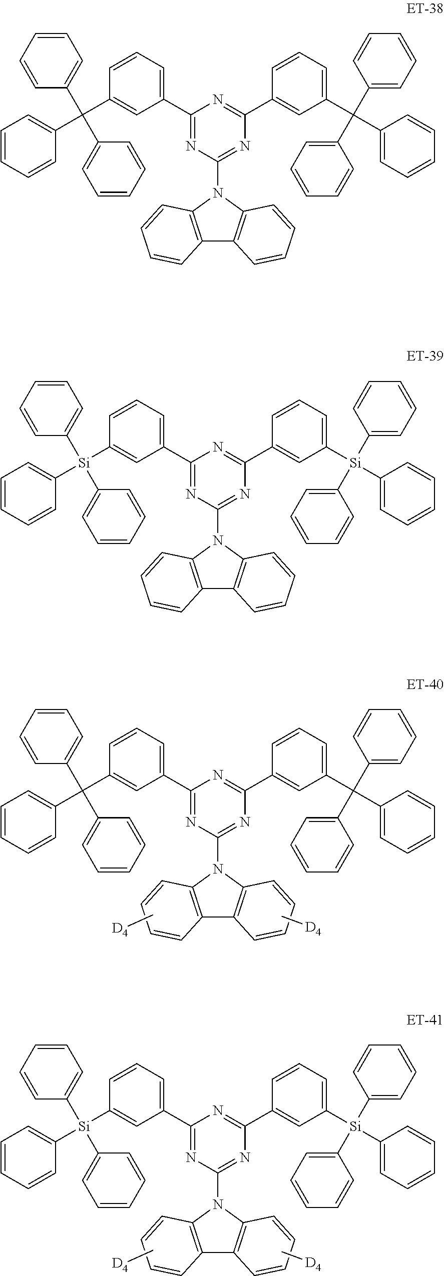

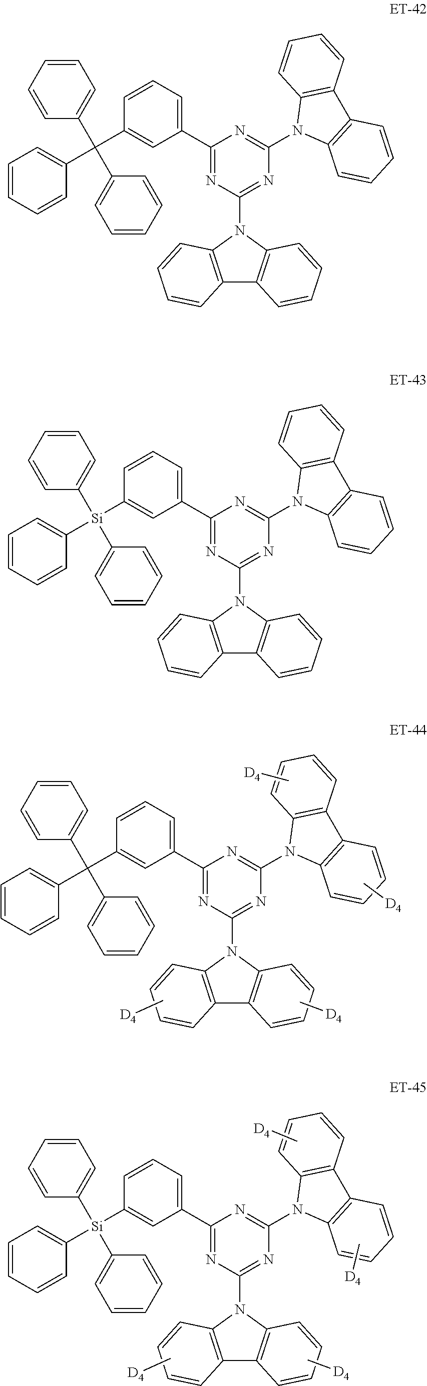

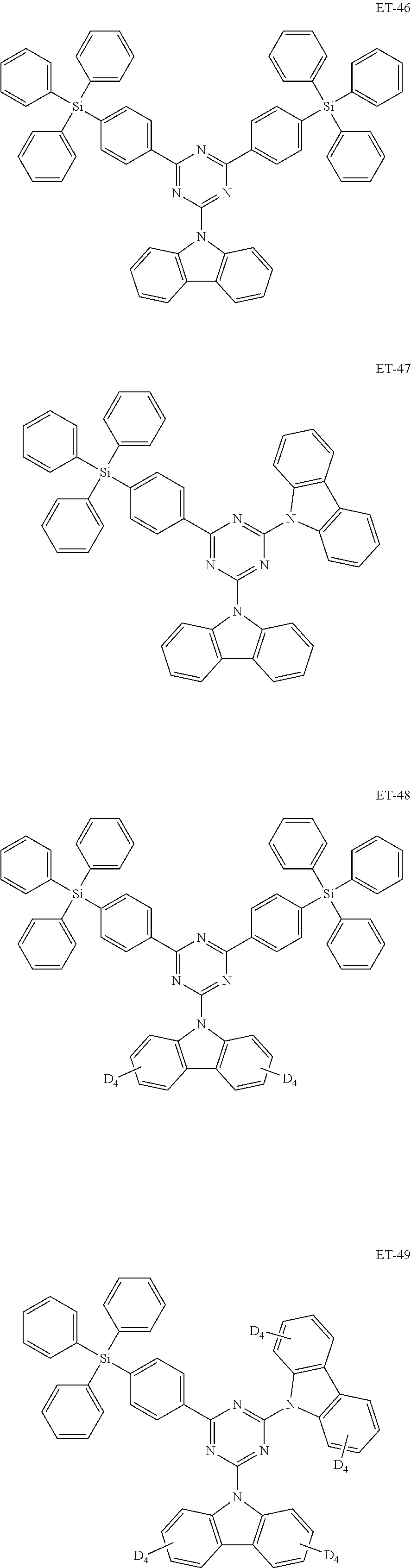

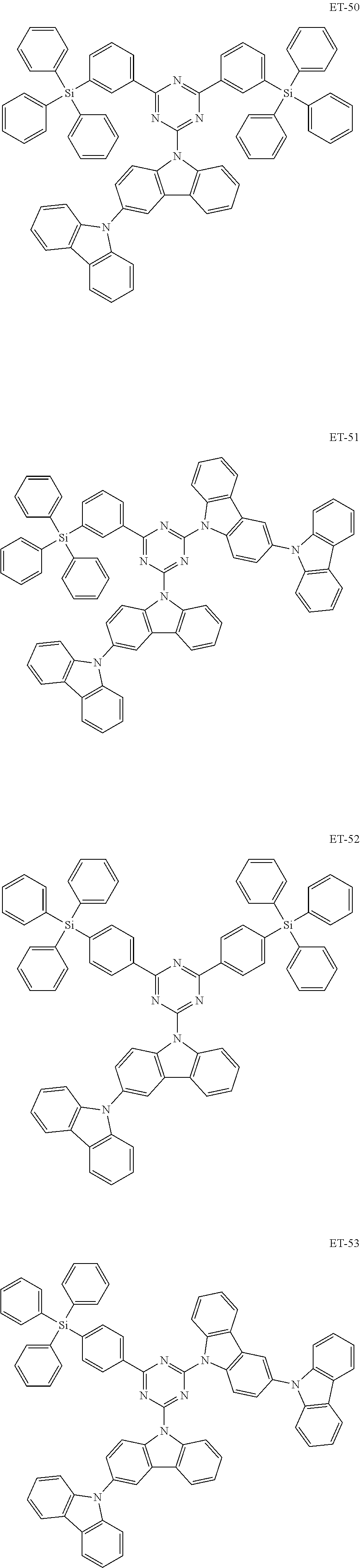

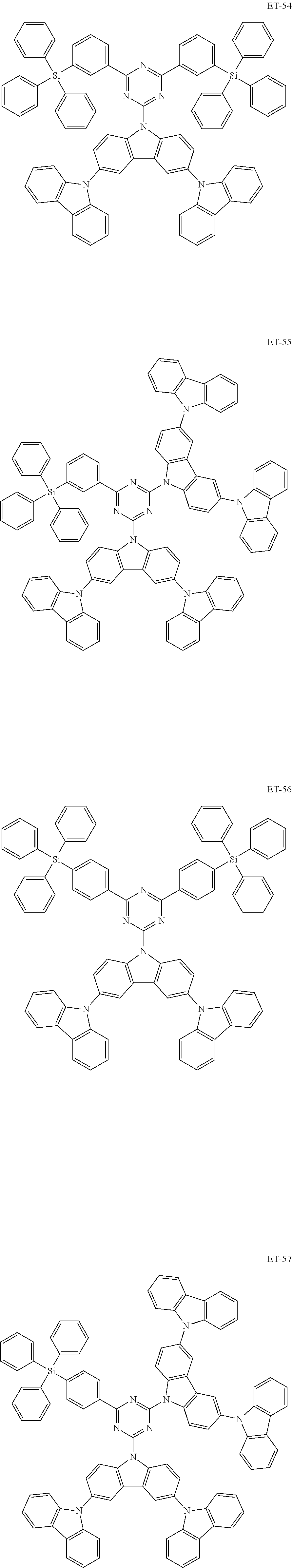

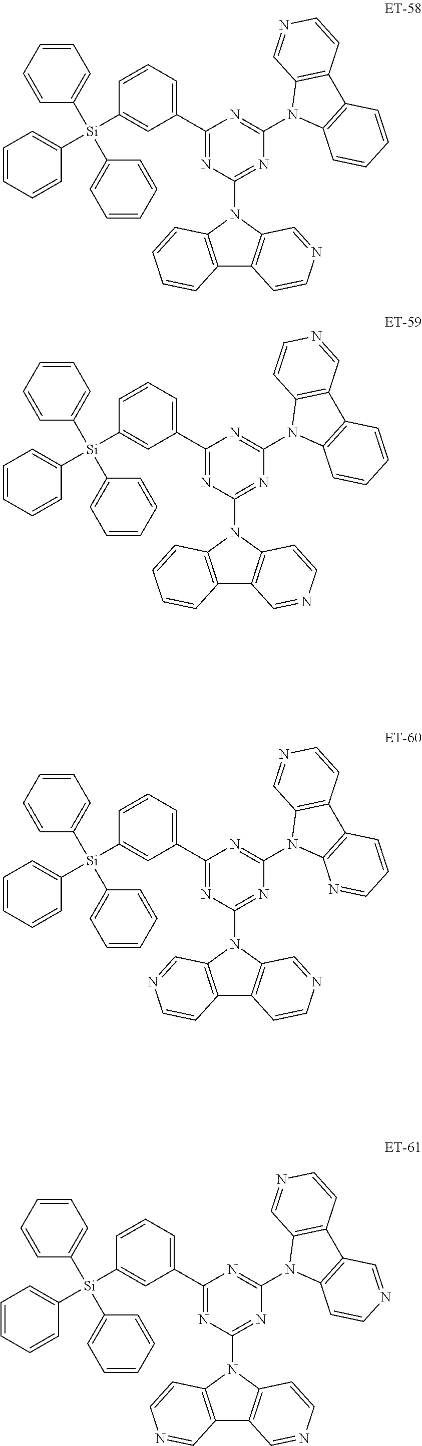 [SMILES: c1ccc([Si](c2ccccc2)(c2ccccc2)c2cccc(-c3nc(-n4c5ccccc5c5ccncc54)nc(-n4c5ccccc5c5ccncc54)n3)c2)cc1 ; c1ccc([Si](c2ccccc2)(c2ccccc2)c2cccc(-c3nc(-n4c5ccccc5c5cnccc54)nc(-n4c5ccccc5c5cnccc54)n3)c2)cc1 ; c1ccc([Si](c2ccccc2)(c2ccccc2)c2cccc(-c3nc(-n4c5cnccc5c5ccncc54)nc(-n4c5cnccc5c5cccnc54)n3)c2)cc1 ; c1ccc([Si](c2ccccc2)(c2ccccc2)c2cccc(-c3nc(-n4c5ccncc5c5cnccc54)nc(-n4c5ccncc5c5ccncc54)n3)c2)cc1]

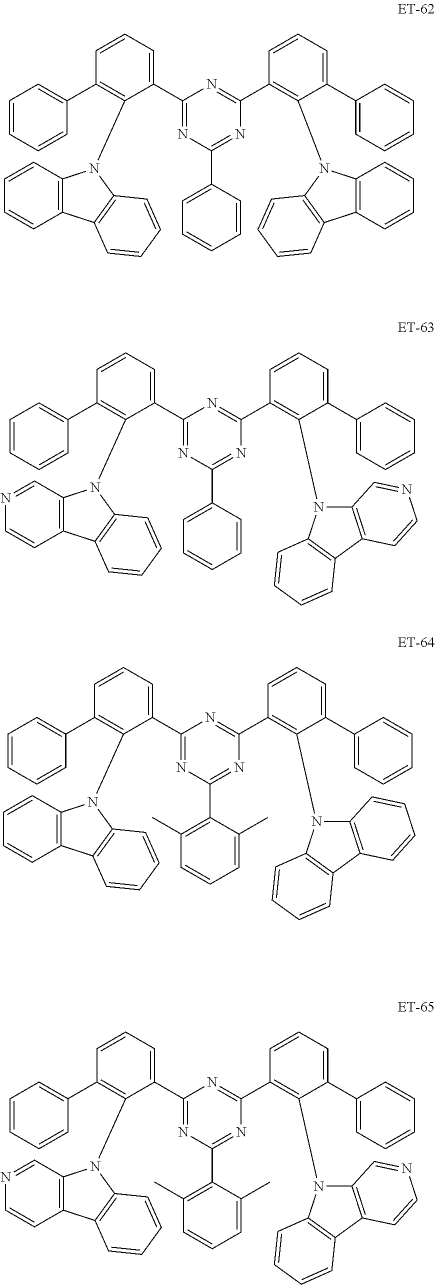 [SMILES: c1ccc(-c2nc(-c3cccc(-c4ccccc4)c3-n3c4ccccc4c4ccccc43)nc(-c3cccc(-c4ccccc4)c3-n3c4ccccc4c4ccccc43)n2)cc1 ; c1ccc(-c2nc(-c3cccc(-c4ccccc4)c3-n3c4ccccc4c4ccncc43)nc(-c3cccc(-c4ccccc4)c3-n3c4ccccc4c4ccncc43)n2)cc1 ; Cc1cccc(C)c1-c1nc(-c2cccc(-c3ccccc3)c2-n2c3ccccc3c3ccccc32)nc(-c2cccc(-c3ccccc3)c2-n2c3ccccc3c3ccccc32)n1 ; Cc1cccc(C)c1-c1nc(-c2cccc(-c3ccccc3)c2-n2c3ccccc3c3ccncc32)nc(-c2cccc(-c3ccccc3)c2-n2c3ccccc3c3ccncc32)n1]

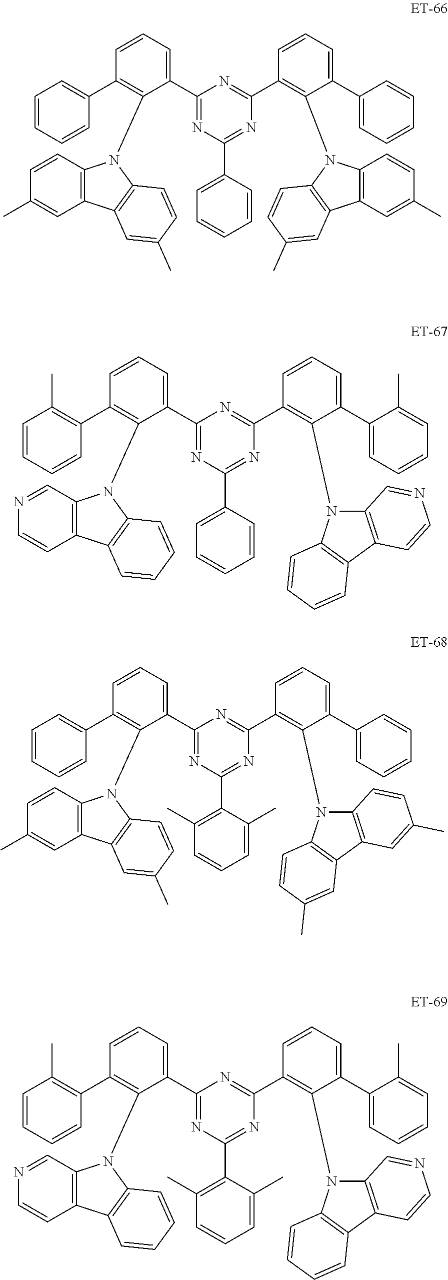

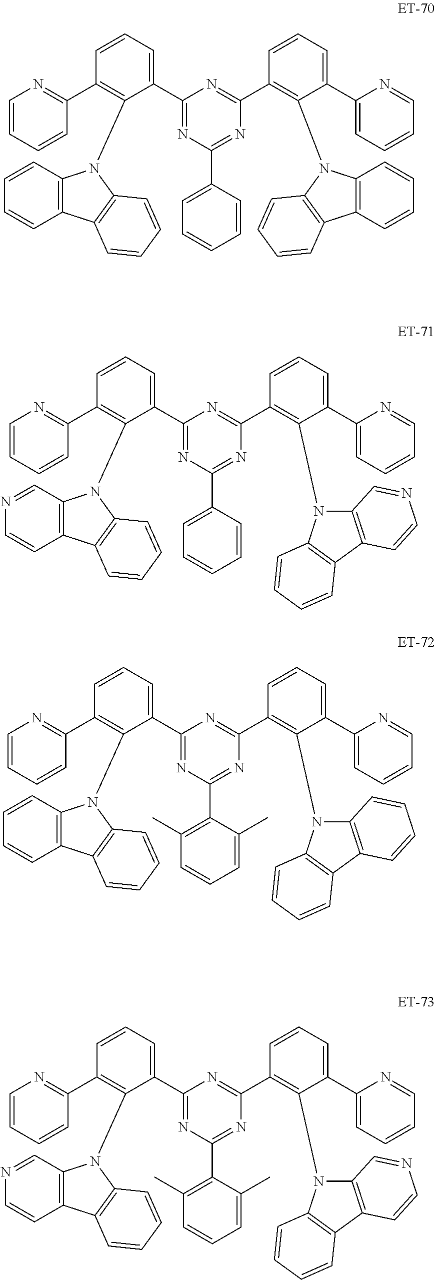 [SMILES: c1ccc(-c2nc(-c3cccc(-c4ccccn4)c3-n3c4ccccc4c4ccccc43)nc(-c3cccc(-c4ccccn4)c3-n3c4ccccc4c4ccccc43)n2)cc1 ; c1ccc(-c2nc(-c3cccc(-c4ccccn4)c3-n3c4ccccc4c4ccncc43)nc(-c3cccc(-c4ccccn4)c3-n3c4ccccc4c4ccncc43)n2)cc1 ; Cc1cccc(C)c1-c1nc(-c2cccc(-c3ccccn3)c2-n2c3ccccc3c3ccccc32)nc(-c2cccc(-c3ccccn3)c2-n2c3ccccc3c3ccccc32)n1 ; Cc1cccc(C)c1-c1nc(-c2cccc(-c3ccccn3)c2-n2c3ccccc3c3ccncc32)nc(-c2cccc(-c3ccccn3)c2-n2c3ccccc3c3ccncc32)n1]

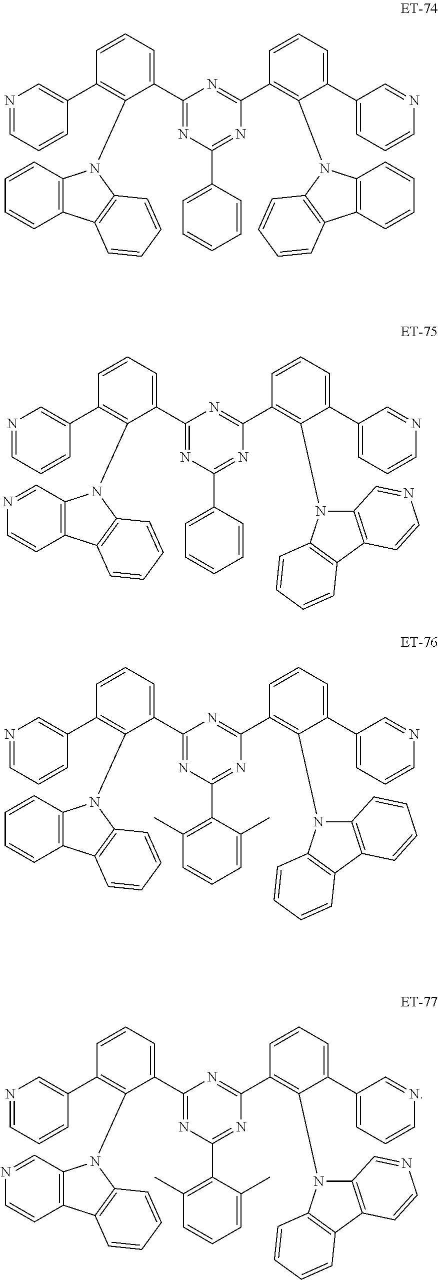 [SMILES: c1ccc(-c2nc(-c3cccc(-c4cccnc4)c3-n3c4ccccc4c4ccccc43)nc(-c3cccc(-c4cccnc4)c3-n3c4ccccc4c4ccccc43)n2)cc1 ; c1ccc(-c2nc(-c3cccc(-c4cccnc4)c3-n3c4ccccc4c4ccncc43)nc(-c3cccc(-c4cccnc4)c3-n3c4ccccc4c4ccncc43)n2)cc1 ; Cc1cccc(C)c1-c1nc(-c2cccc(-c3cccnc3)c2-n2c3ccccc3c3ccccc32)nc(-c2cccc(-c3cccnc3)c2-n2c3ccccc3c3ccccc32)n1 ; Cc1cccc(C)c1-c1nc(-c2cccc(-c3cccnc3)c2-n2c3ccccc3c3ccncc32)nc(-c2cccc(-c3cccnc3)c2-n2c3ccccc3c3ccncc32)n1]

The electron transport layer can be formed together with one or more n-type dopants (such as LiQ, LiTHPh, etc.).

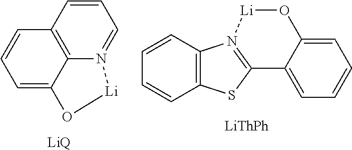

The compounds represented in the embodiment are applied as circularly polarized luminescent materials in the fabrications of OLED devices, and their structures can be represented as follows: on a glass containing ITO, a hole injection layer (HIL) is HT-1: P-3 (95:5 v/v %), with a thickness of 10 nm; a hole transport layer (HTL) is HT-1 with a thickness of 90 nm; a electron barrier layer (EBL) is HT-10 with a thickness of 10 nm, and a luminescent layer (EML) is the main material (H-1 or H-2 or H-3 or H-4 or H-5 or H-6): a Pt(II) or Pd(II) complex (95:5 v/v %) of the present disclosure has a thickness of 35 nm, and a electron transport layer (ETL) is ET-13: LiQ (50:50 v/v %) with a thickness of 35 nm, and a cathode Al with a thickness of 70 nm.

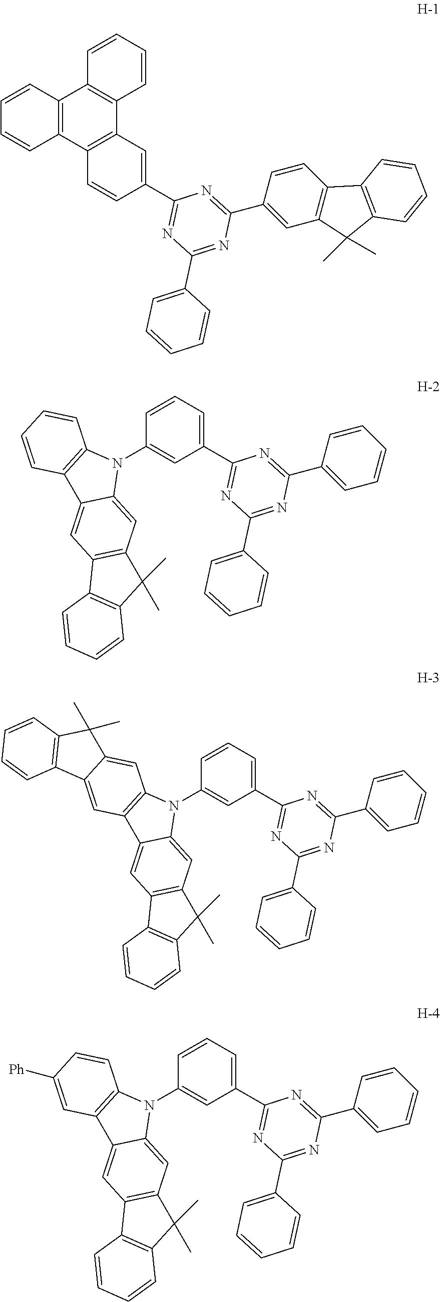

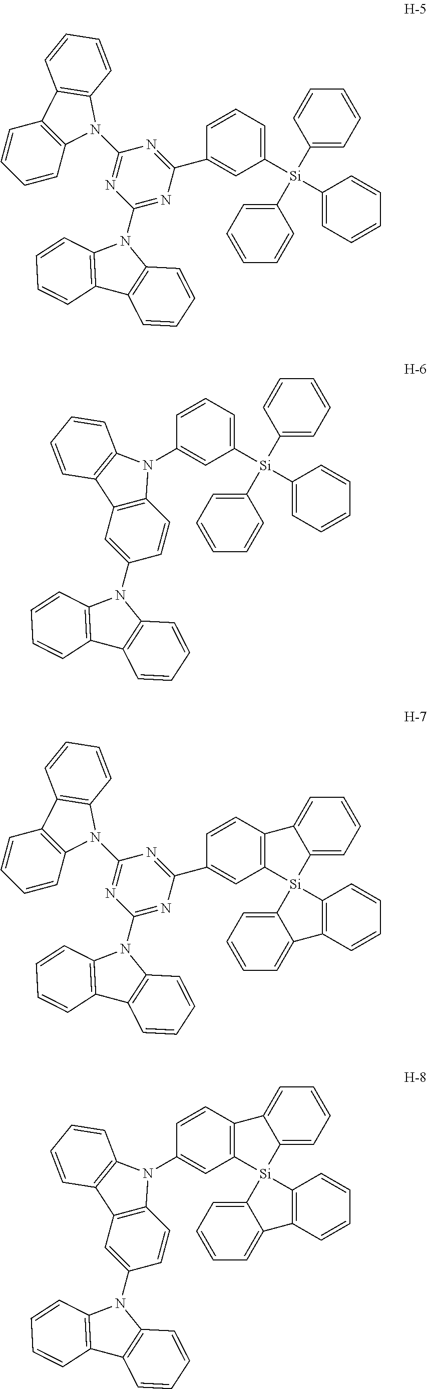 [SMILES: c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2cccc(-n3c4ccccc4c4cc(-n5c6ccccc6c6ccccc65)ccc43)c2)cc1 ; c1ccc2c(c1)-c1ccccc1[Si]21c2ccccc2-c2ccc(-c3nc(-n4c5ccccc5c5ccccc54)nc(-n4c5ccccc5c5ccccc54)n3)cc21 ; c1ccc2c(c1)-c1ccccc1[Si]21c2ccccc2-c2ccc(-n3c4ccccc4c4cc(-n5c6ccccc6c6ccccc65)ccc43)cc21]

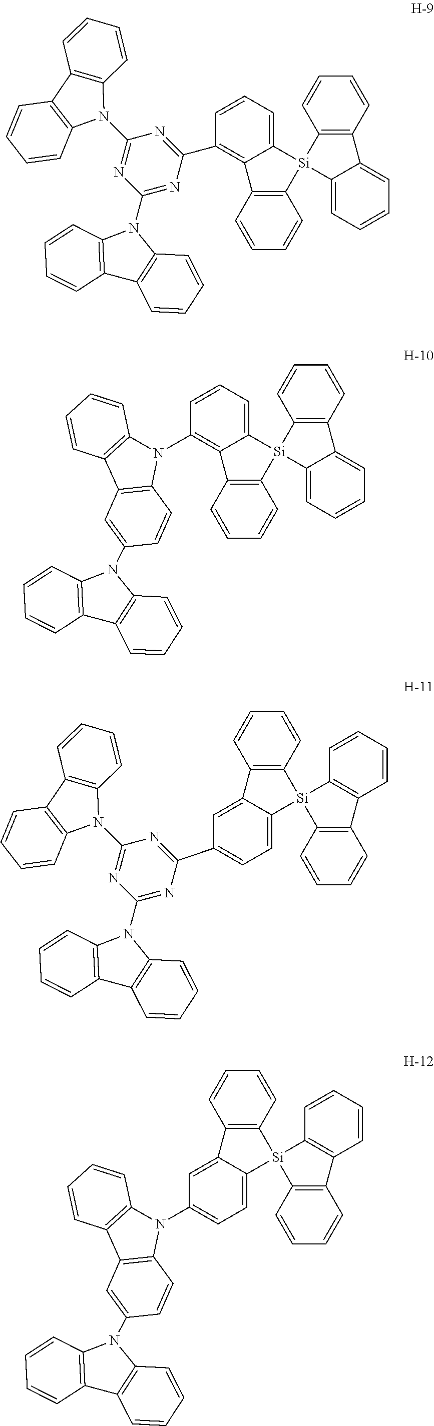 [SMILES: c1ccc2c(c1)-c1ccccc1[Si]21c2ccccc2-c2c(-c3nc(-n4c5ccccc5c5ccccc54)nc(-n4c5ccccc5c5ccccc54)n3)cccc21 ; c1ccc2c(c1)-c1ccccc1[Si]21c2ccccc2-c2c(-n3c4ccccc4c4cc(-n5c6ccccc6c6ccccc65)ccc43)cccc21 ; c1ccc2c(c1)-c1ccccc1[Si]21c2ccccc2-c2cc(-c3nc(-n4c5ccccc5c5ccccc54)nc(-n4c5ccccc5c5ccccc54)n3)ccc21 ; c1ccc2c(c1)-c1ccccc1[Si]21c2ccccc2-c2cc(-n3c4ccccc4c4cc(-n5c6ccccc6c6ccccc65)ccc43)ccc21]

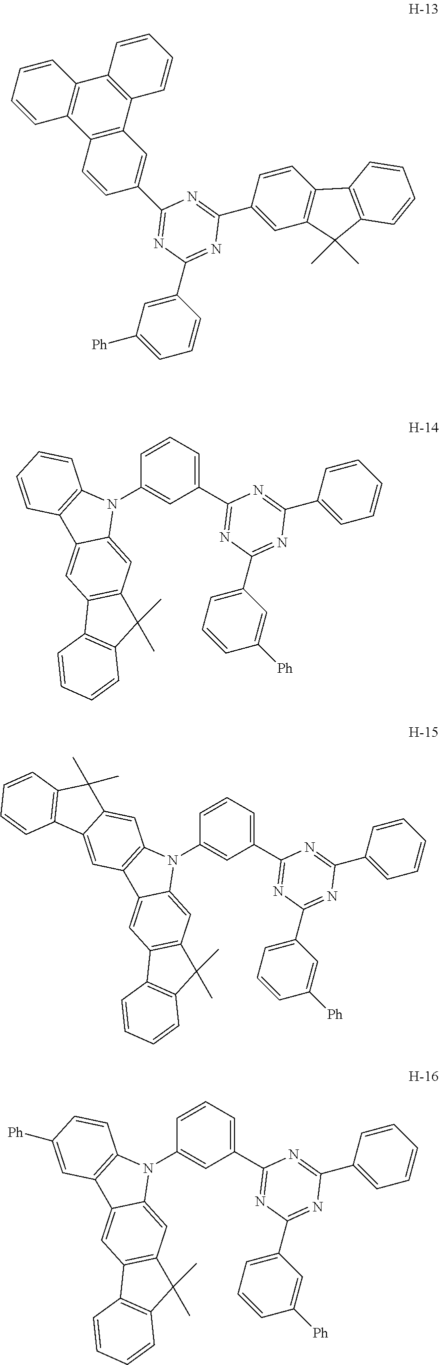 [SMILES: CC1(C)c2ccccc2-c2ccc(-c3nc(-c4cccc(-c5ccccc5)c4)nc(-c4ccc5c6ccccc6c6ccccc6c5c4)n3)cc21 ; CC1(C)c2ccccc2-c2cc3c4ccccc4n(-c4cccc(-c5nc(-c6ccccc6)nc(-c6cccc(-c7ccccc7)c6)n5)c4)c3cc21 ; CC1(C)c2ccccc2-c2cc3c4cc5c(cc4n(-c4cccc(-c6nc(-c7ccccc7)nc(-c7cccc(-c8ccccc8)c7)n6)c4)c3cc21)C(C)(C)c1ccccc1-5 ; CC1(C)c2ccccc2-c2cc3c4cc(-c5ccccc5)ccc4n(-c4cccc(-c5nc(-c6ccccc6)nc(-c6cccc(-c7ccccc7)c6)n5)c4)c3cc21]

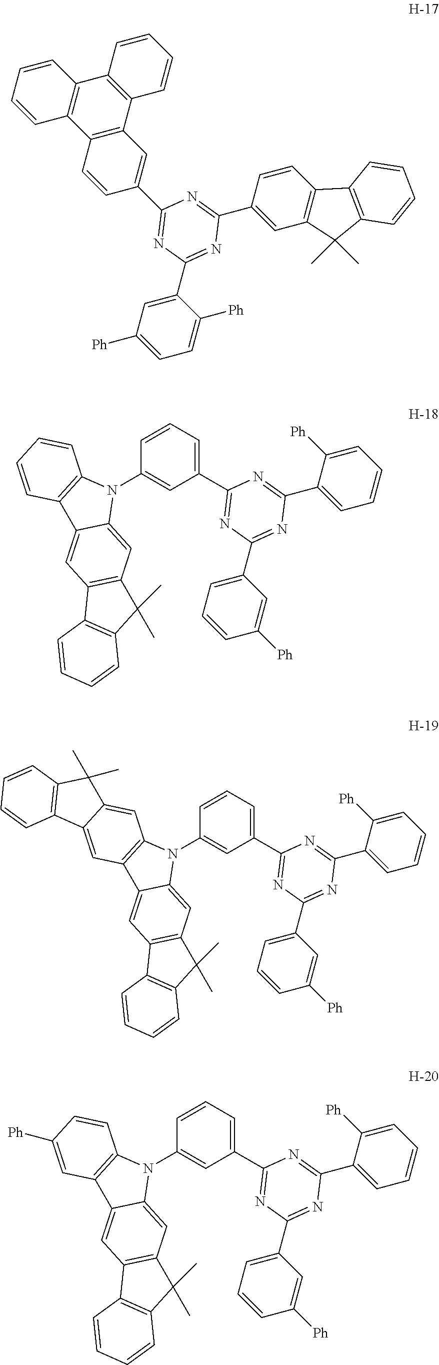 [SMILES: CC1(C)c2ccccc2-c2ccc(-c3nc(-c4ccc5c6ccccc6c6ccccc6c5c4)nc(-c4cc(-c5ccccc5)ccc4-c4ccccc4)n3)cc21 ; CC1(C)c2ccccc2-c2cc3c4ccccc4n(-c4cccc(-c5nc(-c6cccc(-c7ccccc7)c6)nc(-c6ccccc6-c6ccccc6)n5)c4)c3cc21 ; CC1(C)c2ccccc2-c2cc3c4cc5c(cc4n(-c4cccc(-c6nc(-c7cccc(-c8ccccc8)c7)nc(-c7ccccc7-c7ccccc7)n6)c4)c3cc21)C(C)(C)c1ccccc1-5 ; CC1(C)c2ccccc2-c2cc3c4cc(-c5ccccc5)ccc4n(-c4cccc(-c5nc(-c6cccc(-c7ccccc7)c6)nc(-c6ccccc6-c6ccccc6)n5)c4)c3cc21]

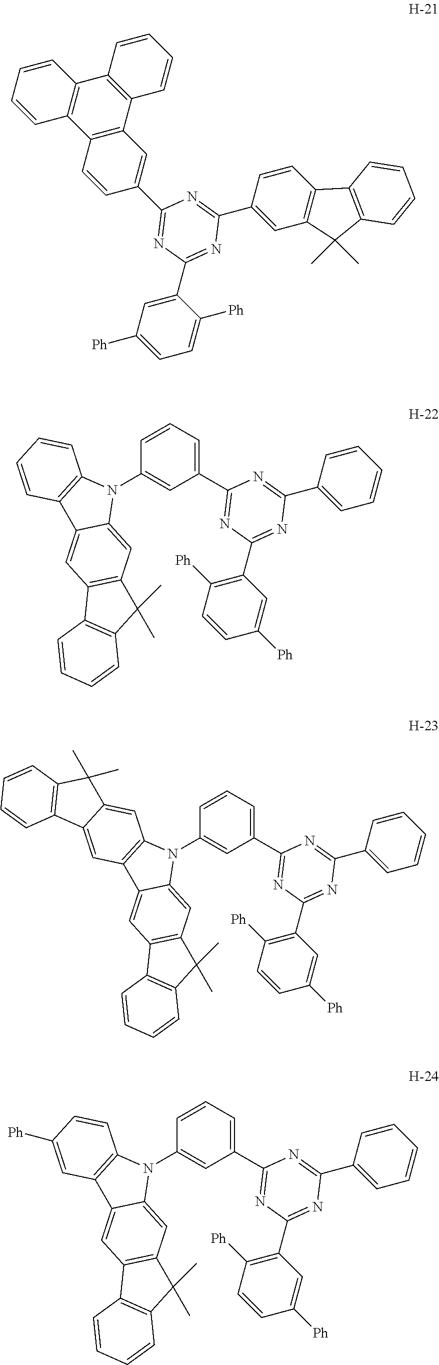 [SMILES: CC1(C)c2ccccc2-c2ccc(-c3nc(-c4ccc5c6ccccc6c6ccccc6c5c4)nc(-c4cc(-c5ccccc5)ccc4-c4ccccc4)n3)cc21 ; CC1(C)c2ccccc2-c2cc3c4ccccc4n(-c4cccc(-c5nc(-c6ccccc6)nc(-c6cc(-c7ccccc7)ccc6-c6ccccc6)n5)c4)c3cc21 ; CC1(C)c2ccccc2-c2cc3c4cc5c(cc4n(-c4cccc(-c6nc(-c7ccccc7)nc(-c7cc(-c8ccccc8)ccc7-c7ccccc7)n6)c4)c3cc21)C(C)(C)c1ccccc1-5 ; CC1(C)c2ccccc2-c2cc3c4cc(-c5ccccc5)ccc4n(-c4cccc(-c5nc(-c6ccccc6)nc(-c6cc(-c7ccccc7)ccc6-c6ccccc6)n5)c4)c3cc21]

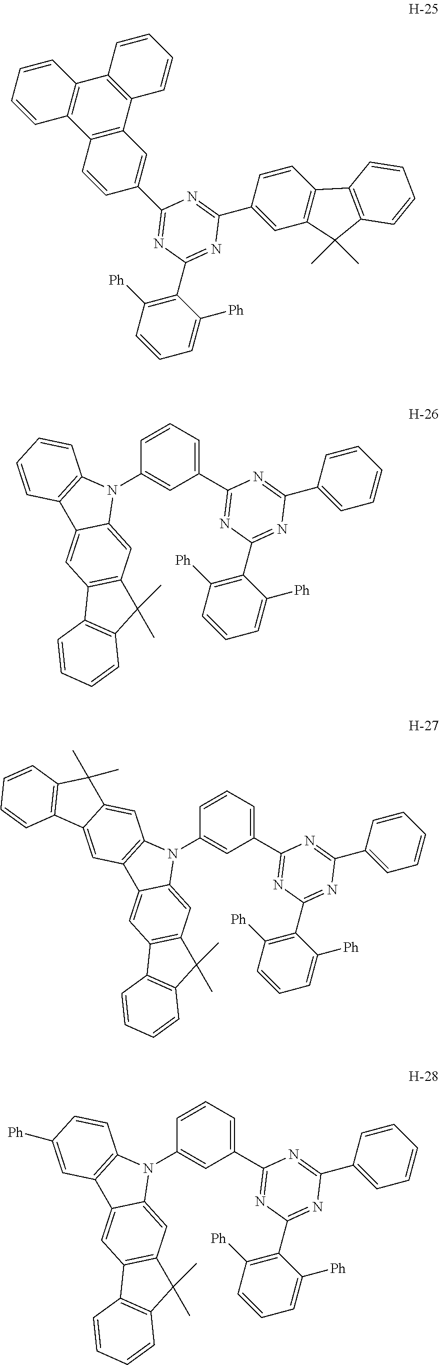 [SMILES: CC1(C)c2ccccc2-c2ccc(-c3nc(-c4ccc5c6ccccc6c6ccccc6c5c4)nc(-c4c(-c5ccccc5)cccc4-c4ccccc4)n3)cc21 ; CC1(C)c2ccccc2-c2cc3c4ccccc4n(-c4cccc(-c5nc(-c6ccccc6)nc(-c6c(-c7ccccc7)cccc6-c6ccccc6)n5)c4)c3cc21 ; CC1(C)c2ccccc2-c2cc3c4cc5c(cc4n(-c4cccc(-c6nc(-c7ccccc7)nc(-c7c(-c8ccccc8)cccc7-c7ccccc7)n6)c4)c3cc21)C(C)(C)c1ccccc1-5 ; CC1(C)c2ccccc2-c2cc3c4cc(-c5ccccc5)ccc4n(-c4cccc(-c5nc(-c6ccccc6)nc(-c6c(-c7ccccc7)cccc6-c6ccccc6)n5)c4)c3cc21]

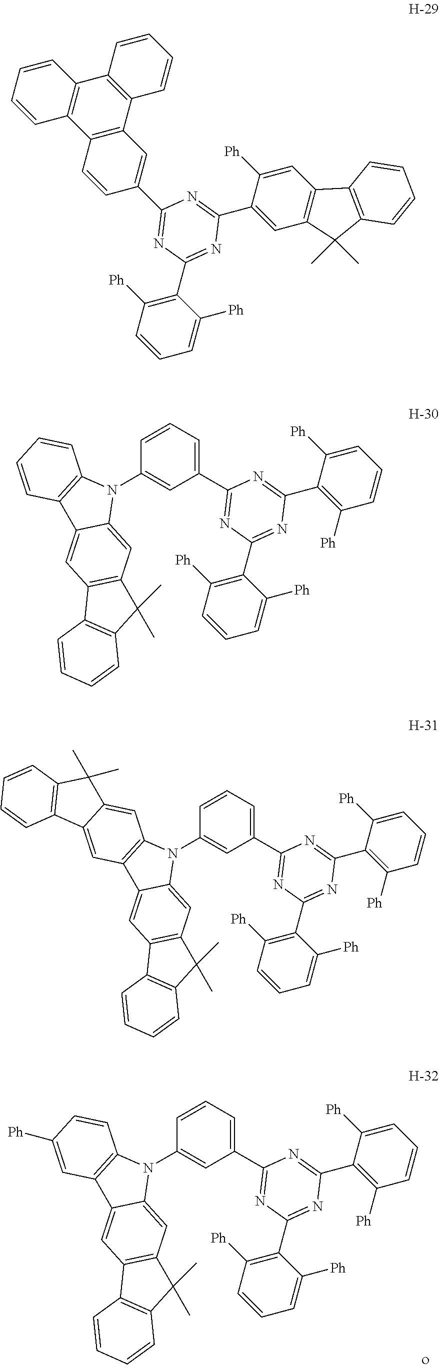

From the electroluminescent devices of deleted materials in Table 4, it can be seen that the helically chiral material molecules in the present disclosure can be used as dopants in the production of circularly polarized light-emitting devices, achieving high external quantum efficiency and strong circularly polarized light-emitting signals.

TABLE 4
Electroluminescence properties of selected helically chiral Pt (II) complexes

| | | λₘₐₓ | EQEₘₐₓ | g_{EL} | | | λₘₐₓ | EQEₘₐₓ | g_{EL} |
|---|---|---|---|---|---|---|---|---|---|
| | dopant | (nm) | (%) | (×10⁻⁴) | | dopant | (nm) | (%) | (×10⁻⁴) |
| comparison | PtON7-tt | 453 | 19 | 0 | | | | | |
| device | | | | | | | | | |
| device 1 | (S,R)-P—PtA1 | 461 | 23 | +5.0 | device 5 | (S)-iBu—P—PtA6 | 461 | 24 | +5.2 |
| device 2 | (S,R)-P—PtA2 | 460 | 24 | +6.2 | device 6 | ((S)-2MeBn—P—PtA9 | 463 | 22 | +4.3 |
| device 3 | (S,S)-P—PtA3 | 465 | 18 | +7.1 | device 7 | (R,S)-P—PtC1 | 510 | 20 | +4.0 |
| device 4 | (S)-iPr—P—PtA4 | 460 | 25 | +4.3 | [symbol] | (R)-P—PtD1 | 459 | 23 | +5.9 |
| device 9 | (S)-2MeiPr—P—PtA5 | 461 | 23 | +4.6 | | | | | |
| device 10 | (S)-2MeiBu—P—PtA7 | 460 | 26 | +3.6 | device 25 | (S)-iBu—P—PtA23 | 458 | 24 | +7.1 |
| device 11 | (S)-Bn—P—PtA8 | 463 | 22 | +4.8 | device 26 | (S)-iBu—P—PtA24 | 463 | 26 | +3.6 |
| device 12 | (S)-Ph—P—PtA10 | 465 | 23 | +2.6 | device 27 | (S,R)-Tjy-P—PtA25 | 462 | 25 | +7.8 |
| device 13 | (R,S)-2Ph—P—PtA11 | 462 | 25 | +4.6 | device 28 | (S,R)-Tjy-P—PtA26 | 465 | 24 | +5.3 |
| device 14 | (S,S)-P—PtA12 | 460 | 21 | +1.6 | device 29 | (S)-iBu—P—PtA27 | 463 | 25 | +5.4 |
| device 15 | (R)-P—PtD1 | 460 | 23 | +6.5 | device 30 | (S)-iPr—P—PtA28 | 462 | 26 | +5.1 |
| device 16 | (S,R)-P—PtF4 | 470 | 19 | +1.8 | device 31 | (S,R)-Tjy-P—PtA29 | 465 | 25 | +8.9 |
| device 17 | (S)-Me—P—PtA13 | 462 | 24 | +1.9 | device 32 | (S)-CMe—P—PtA30 | 452 | 24 | +3.3 |
| device 18 | (S)-iPr—P—PtA14 | 462 | 23 | +4.3 | device 33 | (S)-iBu—P—PtA31 | 461 | 26 | +3.8 |
| device 19 | (S)-tBu—P—PtA15 | 461 | 24 | +3.9 | device 34 | (S)-Cring-iBu—P—PtA32 | 458 | 25 | +3.5 |
| device 20 | (S)-iPr—P—PtA18 | 460 | 25 | +5.6 | device 35 | (R,S)-P—Pt36 | 510 | 23 | +2.6 |
| device 21 | (S)-tBu—P-PtA19 | 461 | 26 | +6.5 | device 36 | (S)-iBu—P—PtA37 | 461 | 25 | +3.3 |
| device 22 | (S)-iPr—P—PtA20 | 460 | 23 | +4.8 | device 37 | (S)-iBuD-P—PtA38 | 462 | 26 | +3.2 |
| device 23 | (S)-iPr—P—PtA21 | 501 | 22 | +6.5 | device 38 | (S)-2MeiBu—P—PtA39 | 463 | 26 | +2.5 |
| device 24 | (S)-iPr—P—PtA22 | 462 | 25 | +6.1 | device 39 | (S)-2MeiBu—P—PtA40 | 462 | 25 | +3.5 |

Note:
λₘₐₓ is the maximum emission wavelength; EQEₘₐₓ is the maximum external quantum efficiency; g_{EL} is the dissymmetric factor of electroluminescence.

It should be noted that the structure is an example of an application of the circularly polarized light emitting material of the present disclosure, and does not limit the specific OLED device structure of the circularly polarized light emitting material shown in the present disclosure. The circularly polarized light emitting material is also not limited to the compound represented in the embodiment.

It can be understood by those skilled in the art that the various embodiments described above are specific embodiments for practicing the disclosure, and that in practice, various changes in form and details may be made therein without departing from the spirit and scope of the disclosure.

For example, many of substituent structures described herein may be replaced with other structures without departing from the spirit of the present disclosure.

1. A helically chiral platinum(II) or palladium(II) complex-based circularly polarized luminescent material, wherein its chemical formula is represented by general formulas (I) and (I′), where (I) and (I′) are enantiomers of each other: 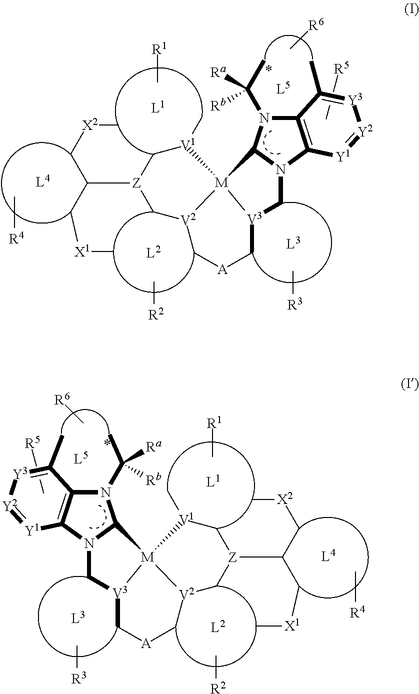
where M is Pt or Pd; V¹, V², and V³ are each independent N or C; Y¹, Y², and Y³ are each independent as N or C;
L¹, L², L³, and L⁴ are each independent as five membered or six membered carbon ring, heterocycle, aromatic ring, or heteroaromatic ring; L⁵ is a six to ten membered carbon ring or heterocycle containing central chirality, where “*” represents a carbon atom with central chirality, that is, R^{a} and R^{b} are different substituents in the same molecule;
A is O, S, CR^{x}R^{y}, C═O, SiR^{x}R^{y}, GeR^{x}R^{y}, NR^{z}, PR^{z}R^{z}P═O, AsR^{z}, R^{z}As═O, S═O, SO₂, Se, Se═O, SeO₂, BH, BR^{z}, R^{z}Bi═O or BiR^{z};
X¹ and X² can exist or not exist, and if they exist, X¹ and X² are each independently single bond, O, S, CR^{x}R^{y}, C═O, SiR^{x}R^{y}, GeR^{x}R^{y}, NR^{z}, PR^{z}, R^{z}P═O, AsR^{z}, R^{z}As═O, S═O, SO₂, Se, Se═O, SeO₂, BH, BR^{z}, R^{z}Bi═O or BiR^{z};
Z is N, CR^{x}, SiR^{x}, GeR^{x}, B, P, P═O, As, As═O, Bi═O or Bi;
R¹, R², R³, R⁴, R⁵ and R⁶ each independently representing single, double, three, four, five, or six substituted or unsubstituted, and meanwhile, R¹, R², R³, R⁴, R⁵, R⁶, R^{a}, R^{b}, R^{x}, R^{y} and R^{z} are each independently hydrogen, deuterium, halogen, alkyl, cycloalkyl, aryl, heterocycloalkyl, heteroaryl, haloalkyl, haloaryl, haloheteroaryl, alkoxy, aryloxy, alksiliyl, arylsiliyl, heteroarylsiliyl, alkyl (hetero) arylsiliyl, alkenyl, cycloalkenyl, alkynyl, hydroxyl, mercapto, nitro, cyano, amino, mono- or dialkylamino, mono- or diarylamino, ester, isonitrile, alkoxycarbonyl, amido, alkoxycarbonylamino, aryloxycarbonylamino, sulfonylamino, sulfamoyl, carbamoyl, alkylthio, sulfinyl, ureido, phosphonamido, imino, sulfo, carboxyl, hydrazino, or combinations thereof, two or more adjacent R¹, R², R³, R⁴, R⁵ and R⁶ can selectively connect to form a fused ring; any two groups in R^{a}, R^{b}, and R⁶ can be connected to form a cyclic system.

2. The helically chiral platinum (II) and palladium (II) complex circularly polarized luminescent material according to claim 1, wherein L³ in the general formula structure is the following structure: 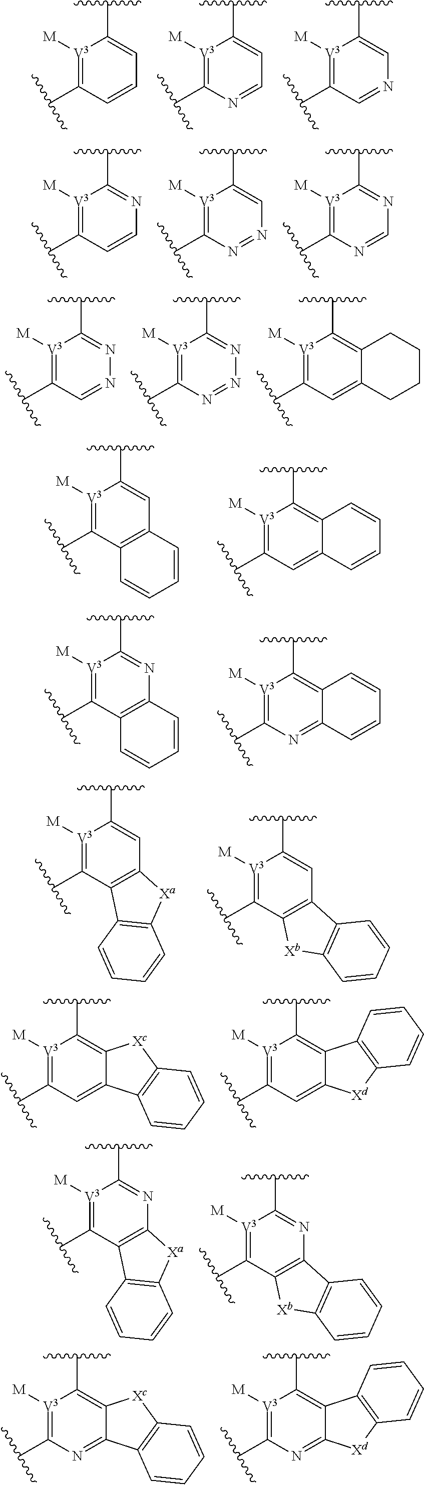 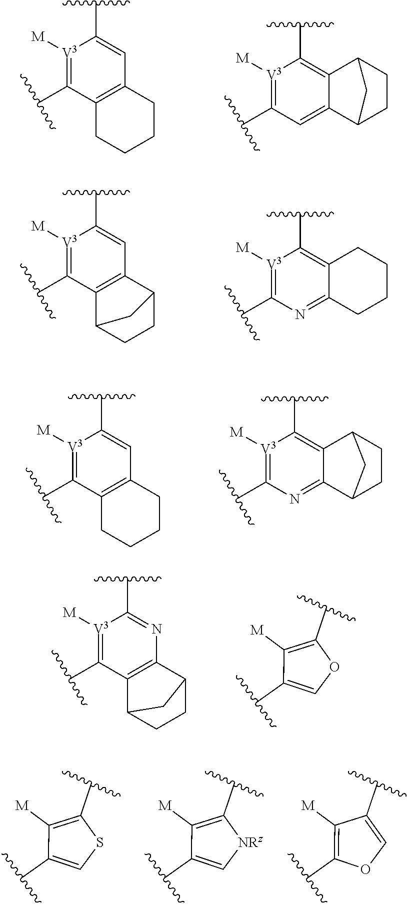 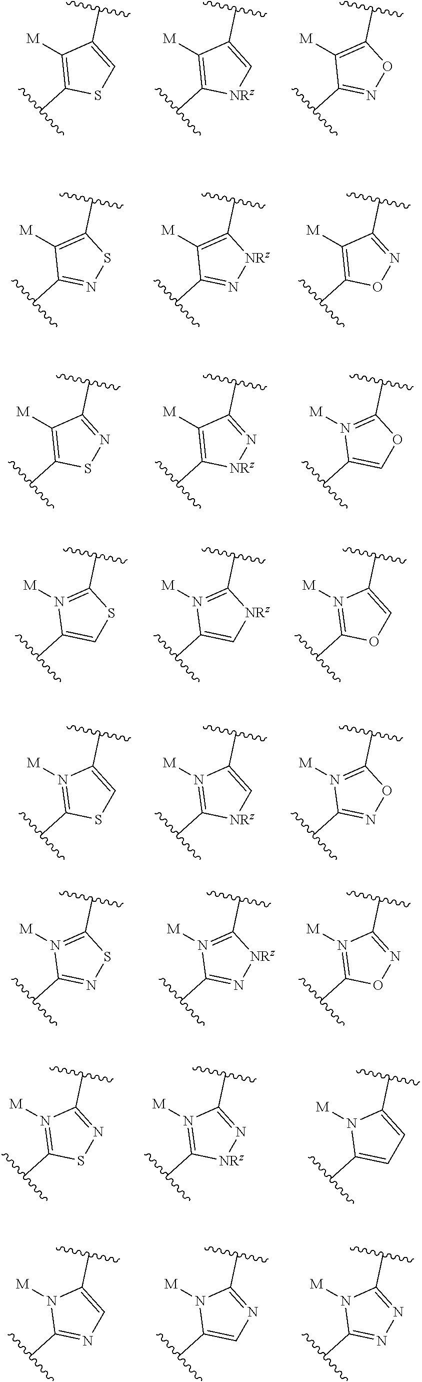 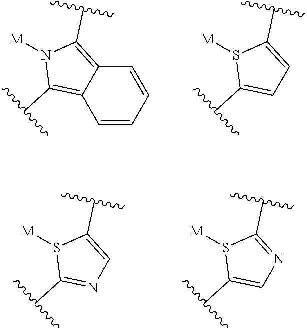
wherein, X^{a}, X^{b}, X^{c}, and X^{d} are each independently single bond, O, S, CR^{x}R^{y}, C═O, SiR^{x}R^{y}, GeR^{x}R^{y}, NR^{z}, PR^{z}, R^{z}P═O, AsR^{z}, R^{z}As═O, S═O, SO₂, Se, Se═O, SeO₂, BH, BR^{z}, R^{z}Bi═O or BiR^{z}.

3. The helically chiral platinum (II) and palladium (II) complex circularly polarized luminescent material according to claim 1, wherein its chemical formula is the following general formulas (I-A), (I-B), (I-C), (I-D), (I-E), (I-F), (I-G), (I-H), (I-I) and their enantiomers (I′-A), (I′-B), (I′-C), (I′-D), (I′-E), (I′-F), (I′-G), (I′-H), (I′-I)□ 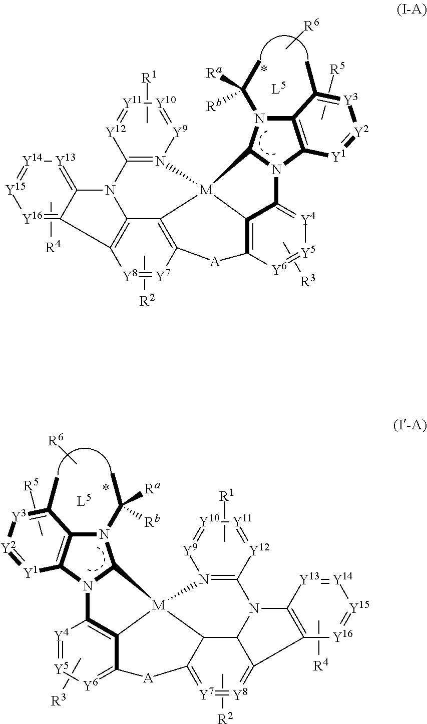 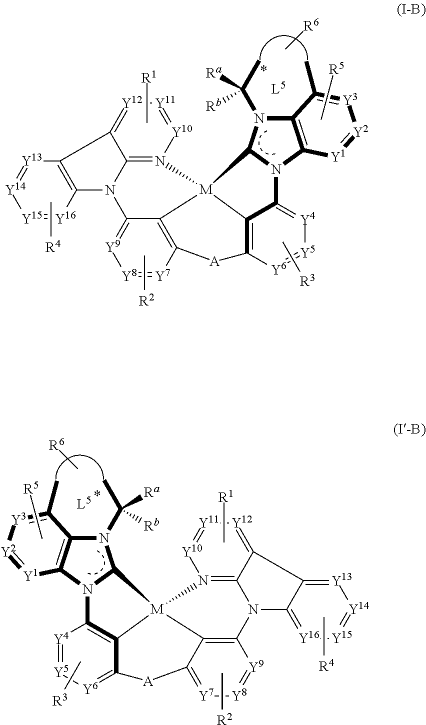 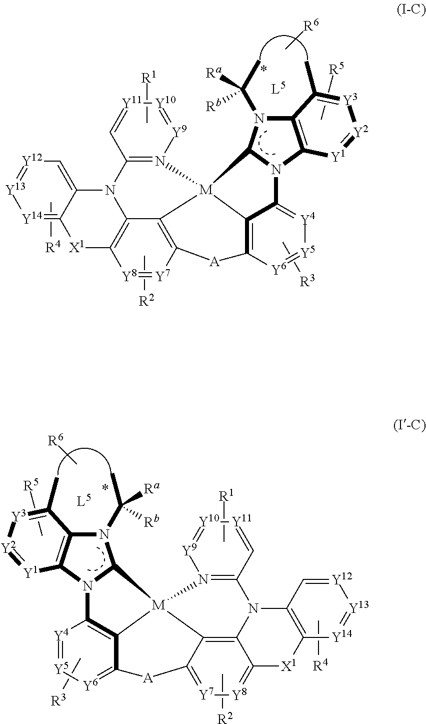 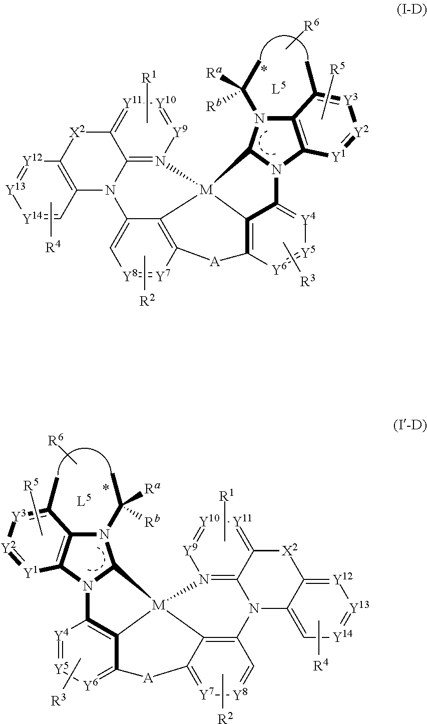 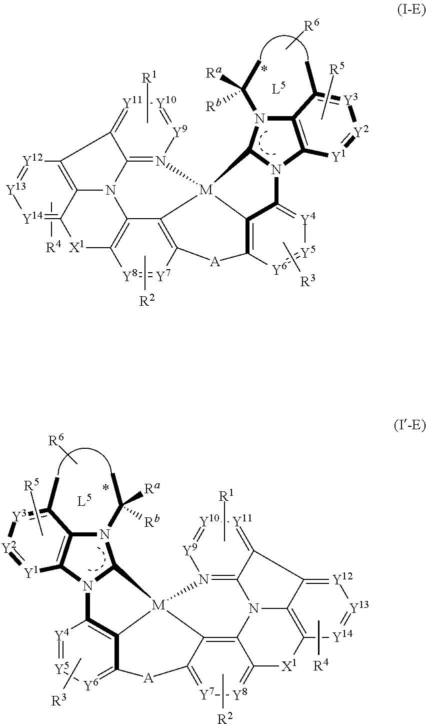 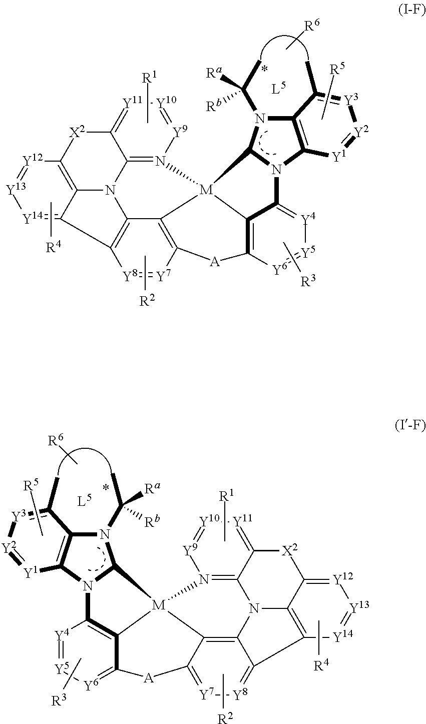 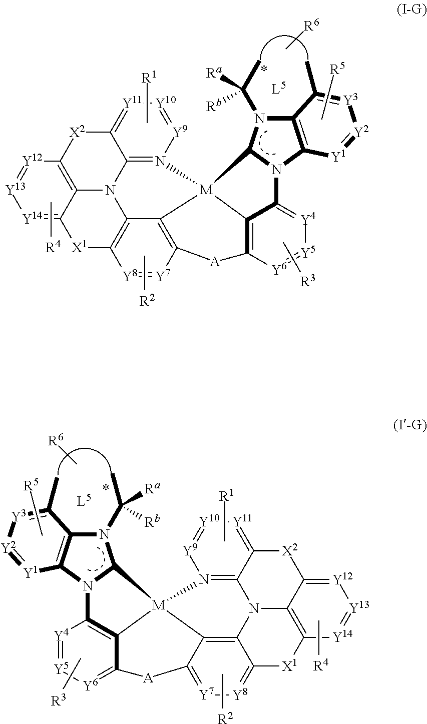 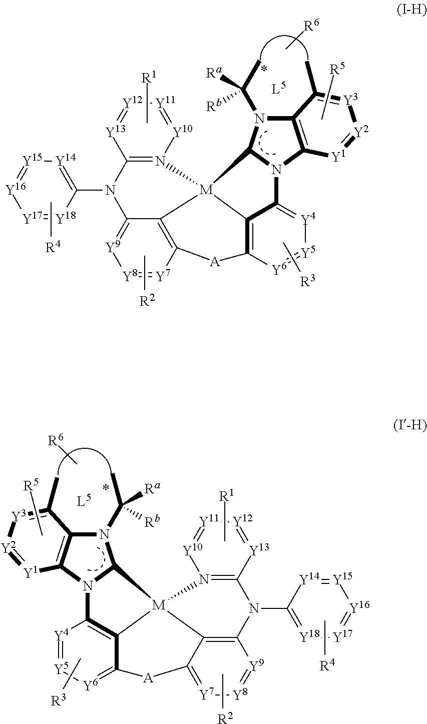 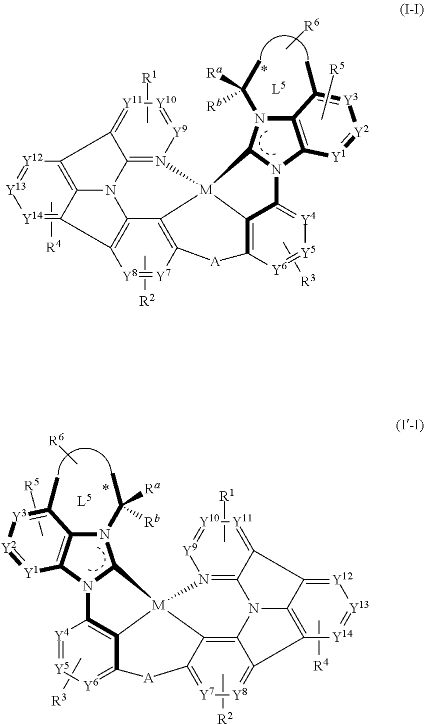
Y⁴, Y⁵, Y⁶, Y⁷, Y⁸, Y⁹, Y¹⁰, Y¹¹, Y¹², Y¹³, Y¹⁴, Y¹⁵ and Y¹⁶ are each independently N or C.

4. The helically chiral platinum (II) and palladium (II) complex circularly polarized luminescent material according to claim 1, wherein L⁵ in the general structure can be the following structures and their enantiomers: 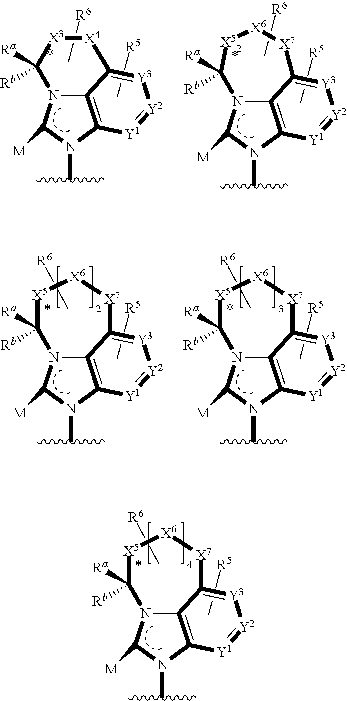
wherein X³, X⁴, X⁵, X⁶ and X⁷ are each independently O, S, CR^{x}R^{y}, 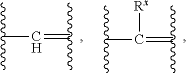
C═O, SiR^{x}R^{y}, GeR^{x}R^{y}, NR^{z}, PR^{z}, R^{z}P═O, AsR^{z}, R^{z}As═O, S═O, SO₂, Se, Se═O, SeO₂, BH, BR^{z}, R^{z}Bi═O or BiR^{z}.

5. The helically chiral platinum (II) and palladium (II) complex circularly polarized luminescent material according to claim 1, wherein L⁵ in the general structure is the following structures and their enantiomers: 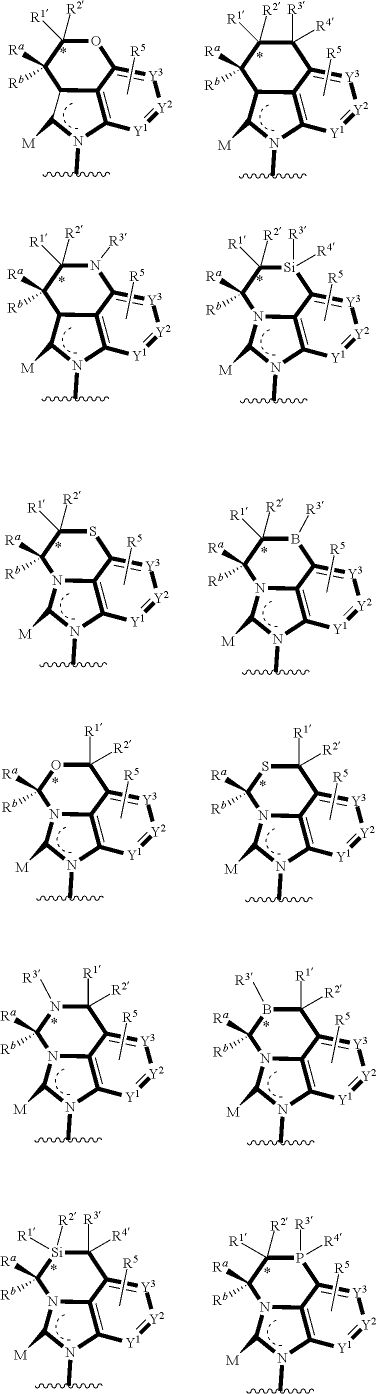 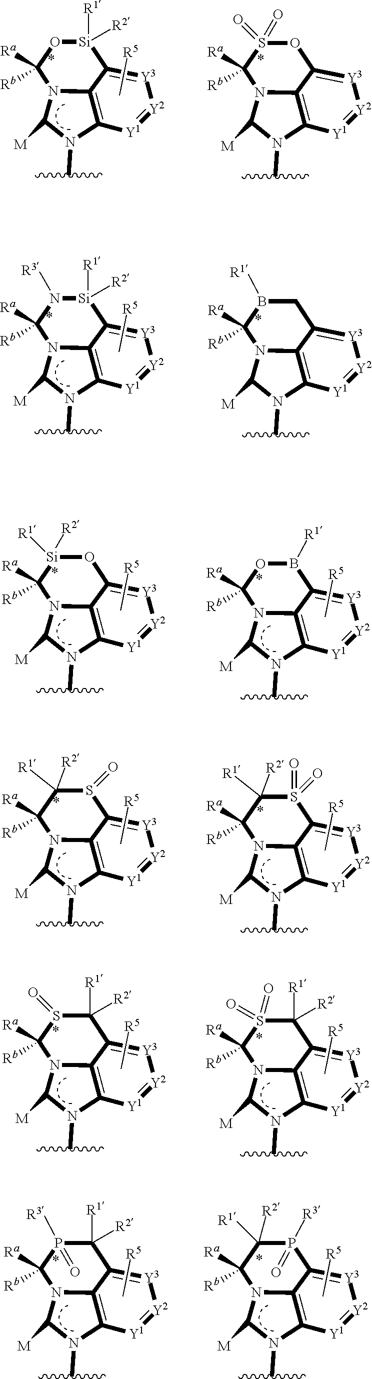 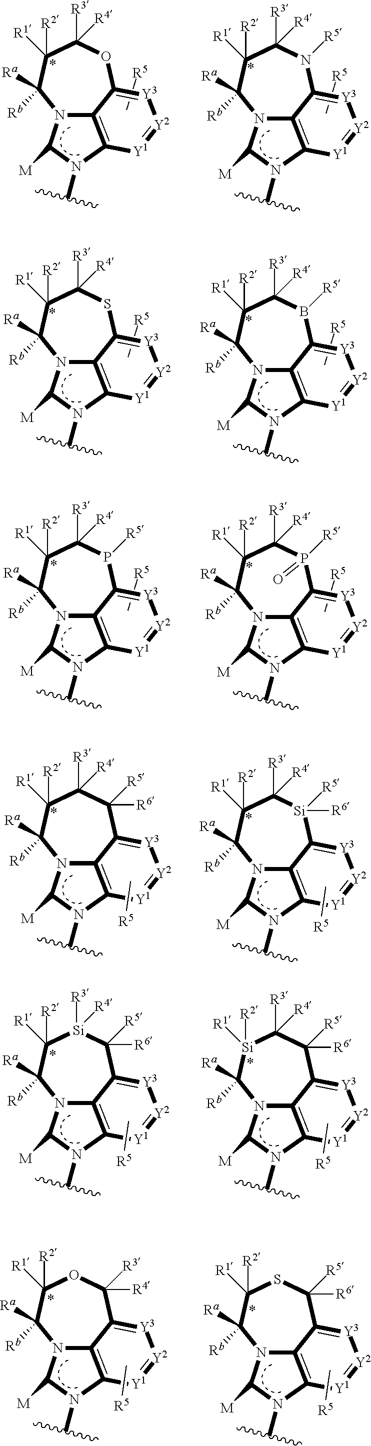 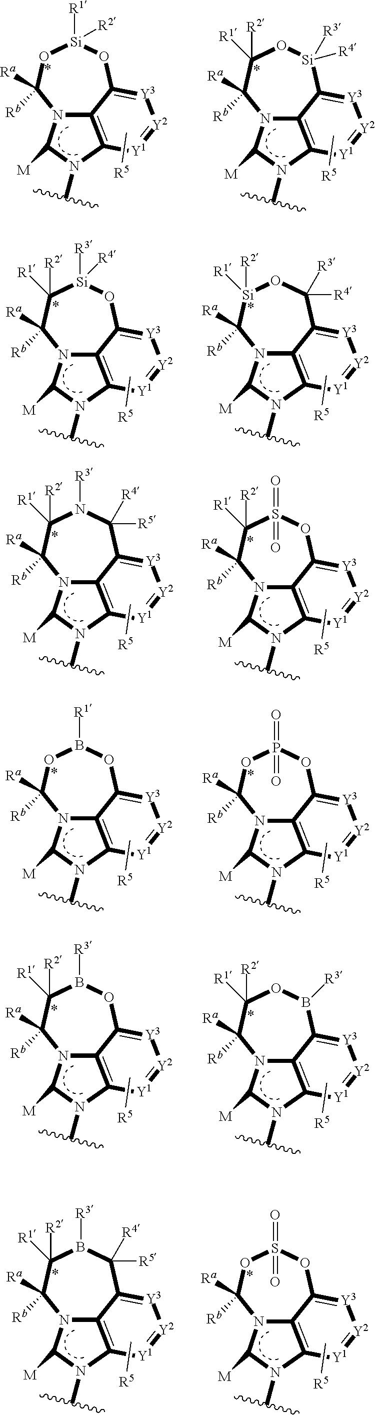 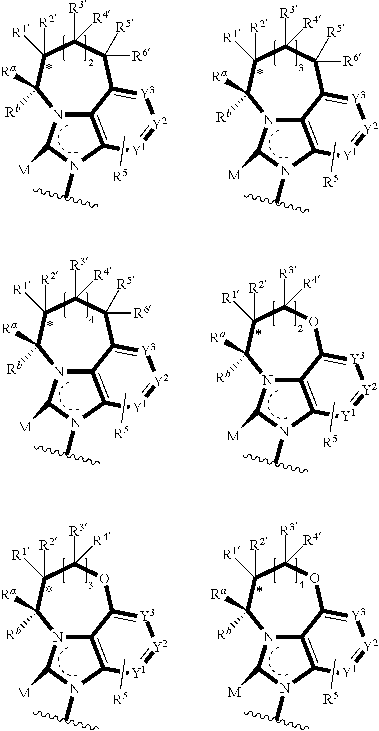
wherein, R^{1′}, R^{2′}, R^{3′}, R^{4′}, R^{5′} and R^{6′} are each independently hydrogen, deuterium, halogen, alkyl, cycloalkyl, aryl, heterocycloalkyl, heteroaryl, haloalkyl, haloaryl, haloheteroaryl, alkoxy, aryloxy, alksiliyl, arylsiliyl, heteroarylsiliyl, alkyl (hetero) arylsiliyl, alkenyl, cycloalkenyl, alkynyl, hydroxyl, mercapto, nitro, cyano, amino, mono- or dialkylamino, mono- or diarylamino, ester, isonitrile, alkoxycarbonyl, amido, alkoxycarbonylamino, aryloxycarbonylamino, sulfonylamino, sulfamoyl, carbamoyl, alkylthio, sulfinyl, ureido, phosphonamido, imino, sulfo, carboxyl, hydrazino, or combinations thereof; two or more adjacent R^{1′}, R^{2′}, R^{3′}, R^{4′}, R^{5′} and R^{6′} can selectively connect to form a fused ring.

6. The helically chiral platinum (II) and palladium (II) complex circularly polarized luminescent material according to claim 5, wherein L⁵ in the general structure is the following structures and their enantiomers: 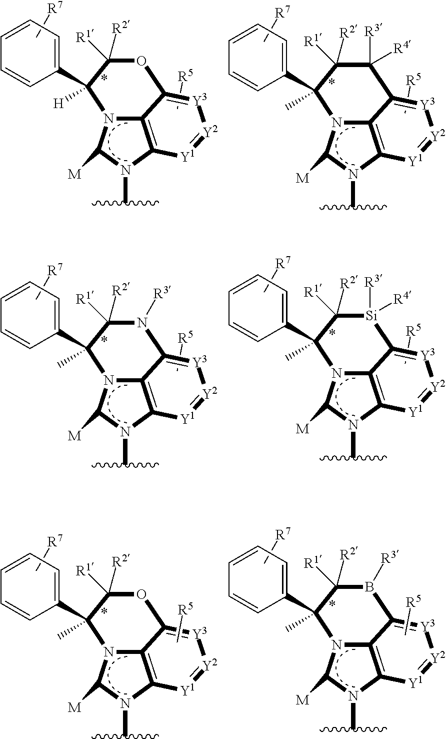 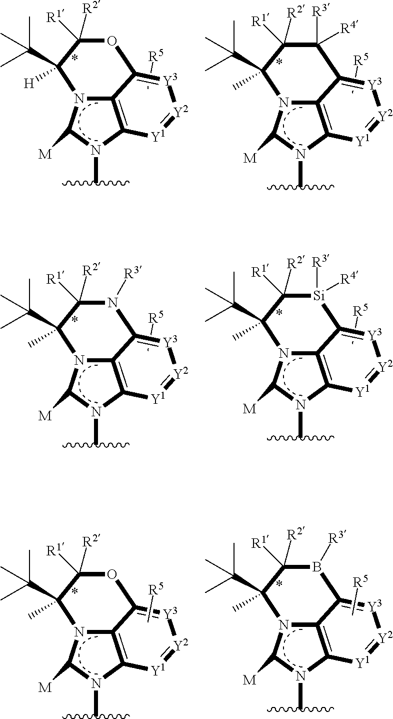 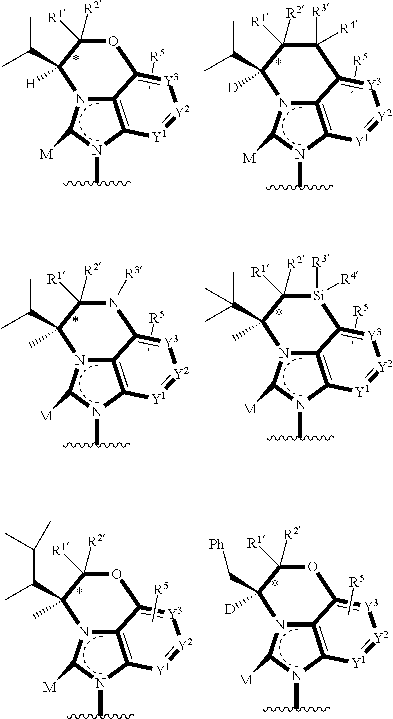 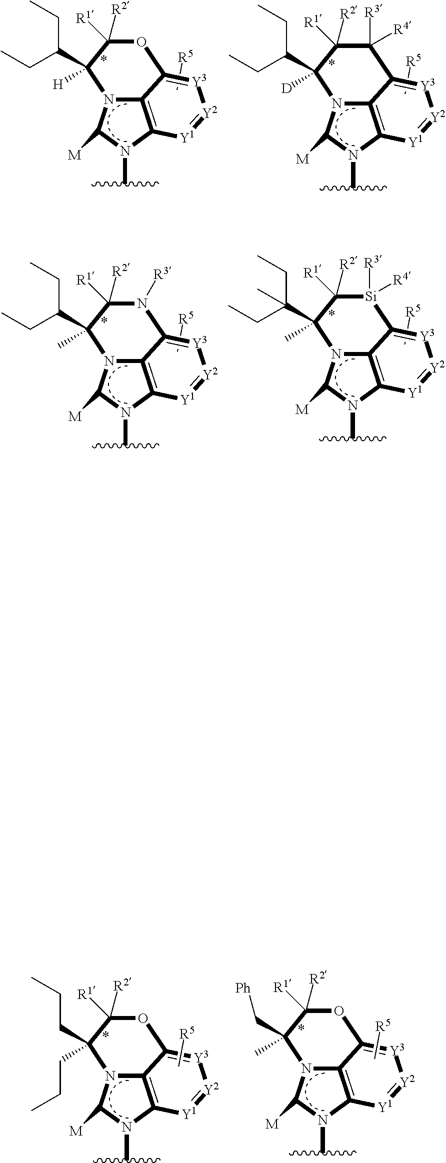 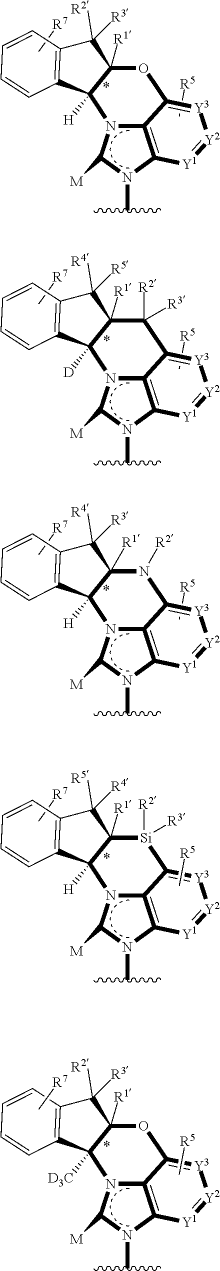 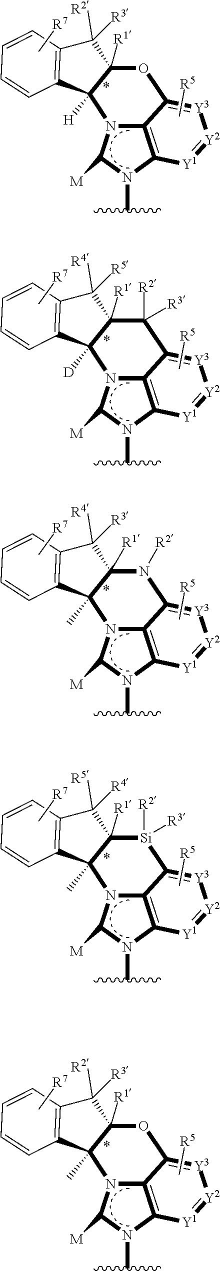 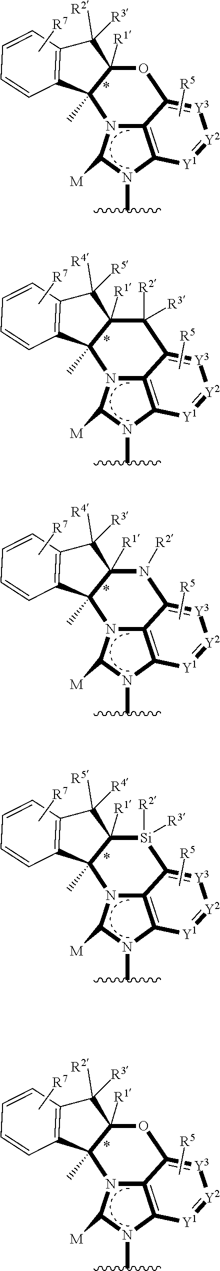 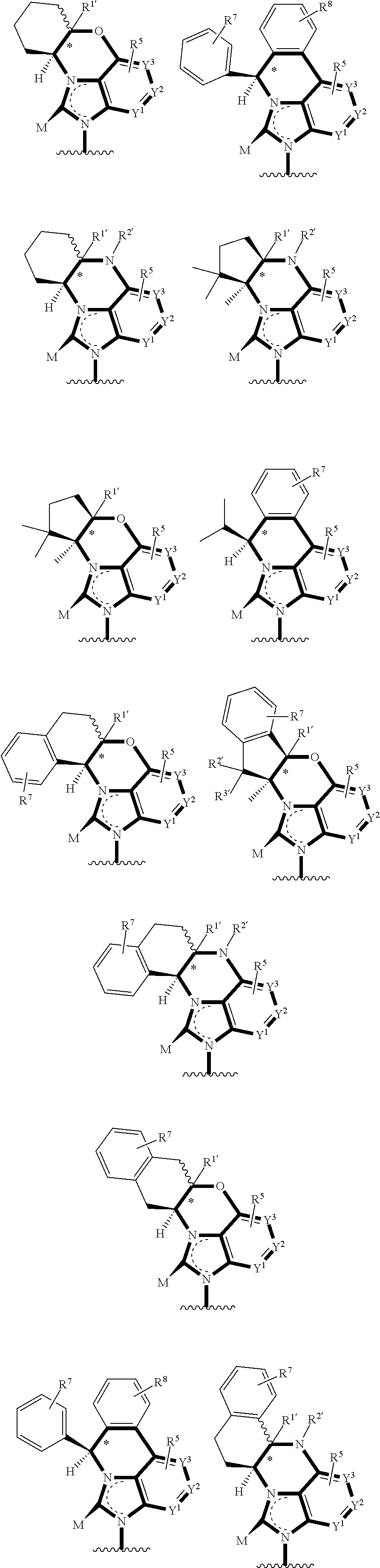 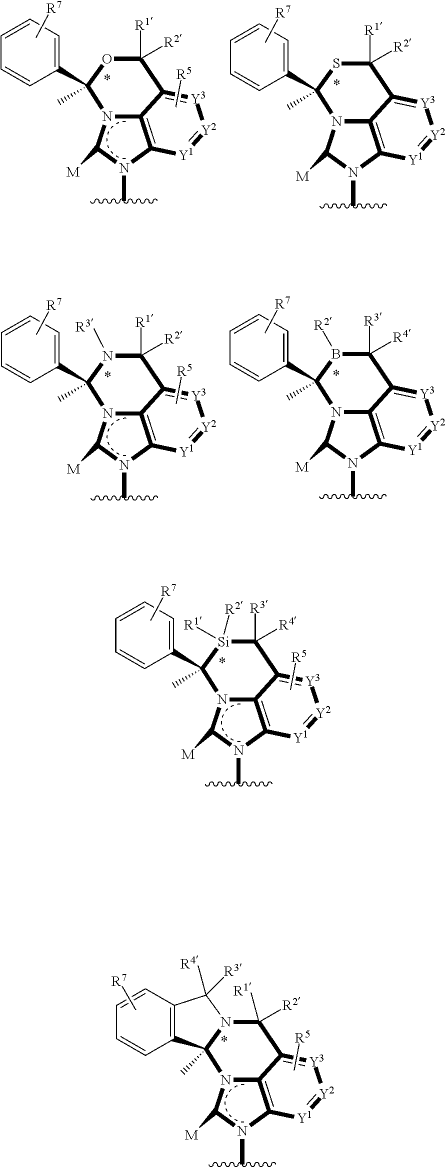 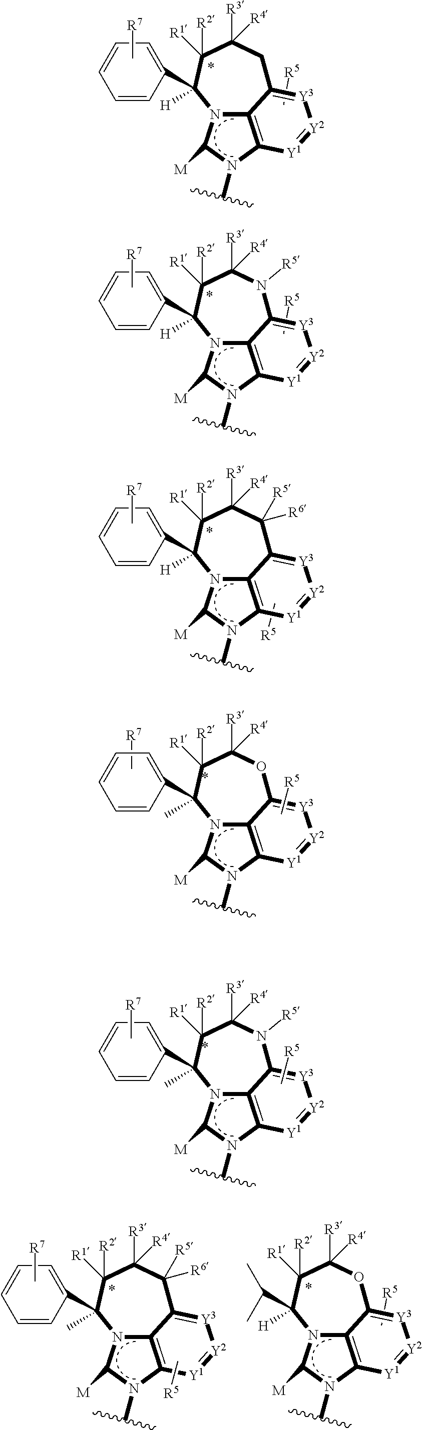 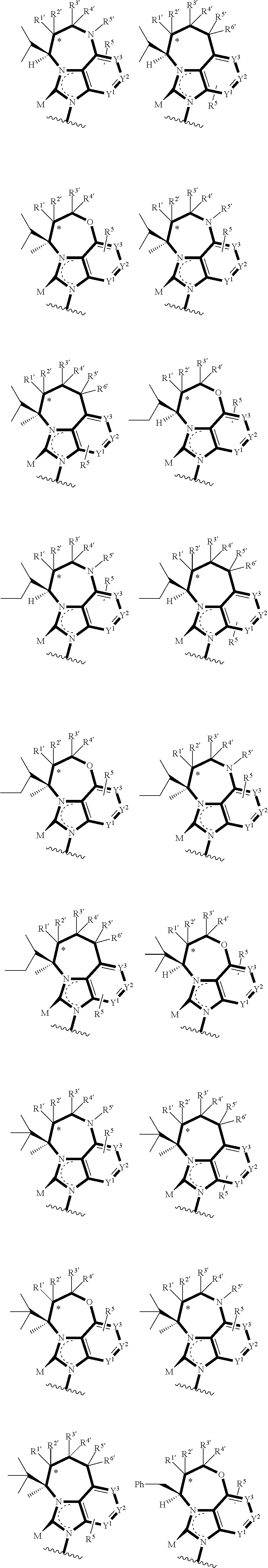 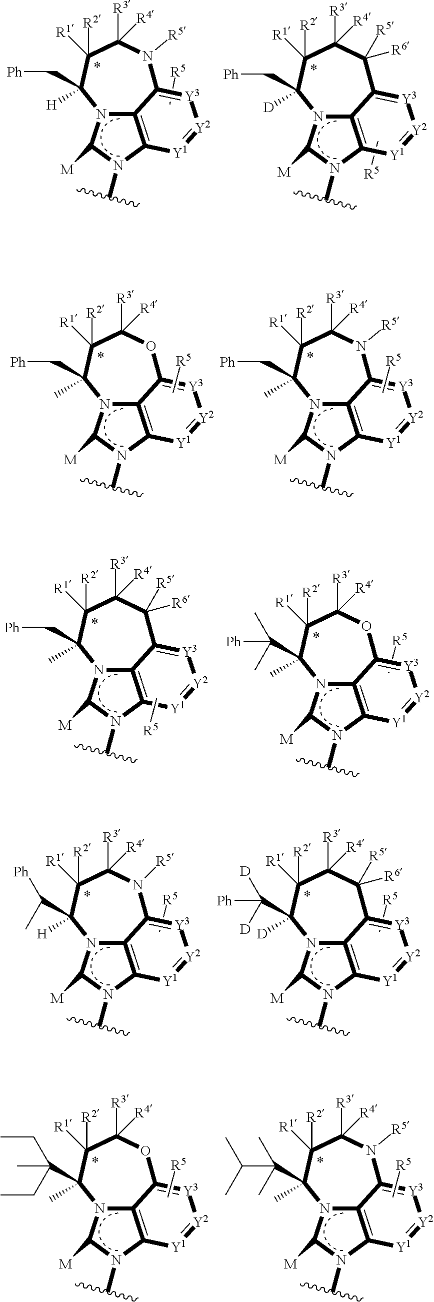 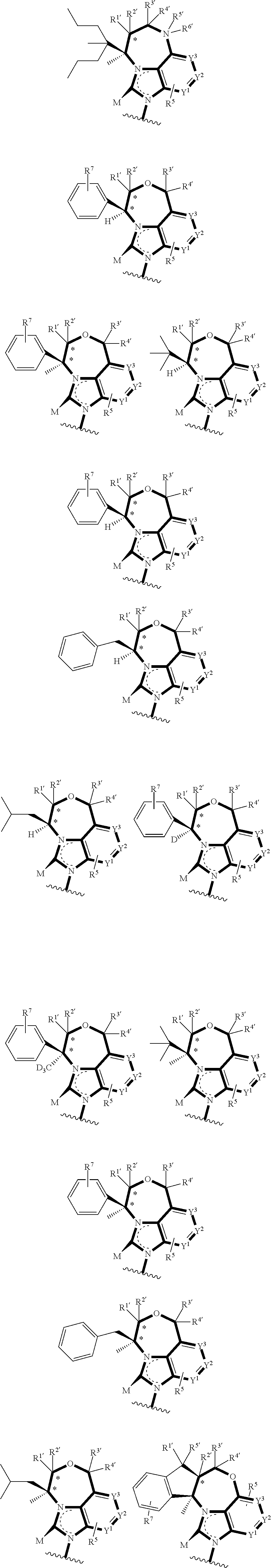 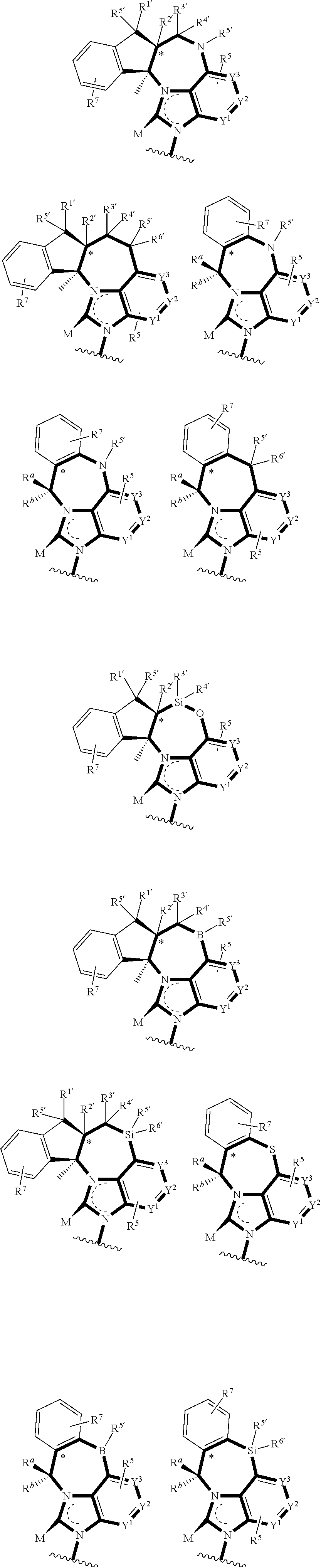 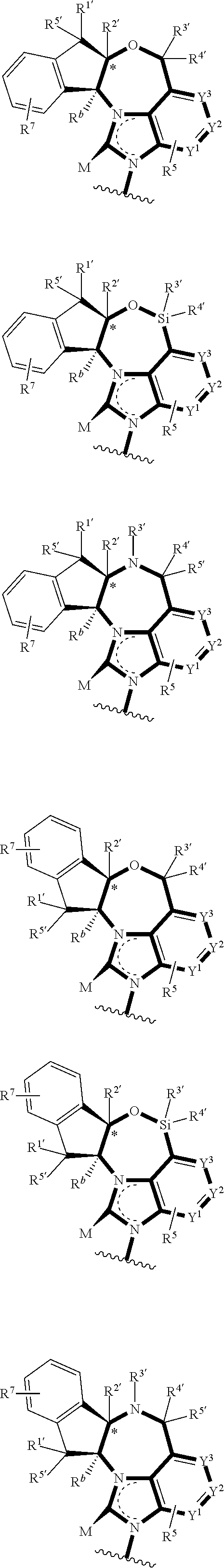 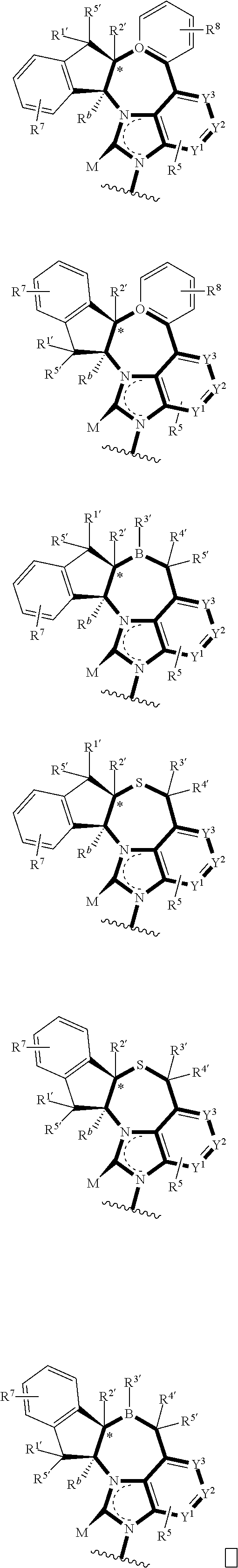
R⁷ and R⁸ each independently representing single, double, three, four, or five substituted or unsubstituted, and meanwhile, R⁷ and R⁸ are each independently hydrogen, deuterium, halogen, alkyl, cycloalkyl, aryl, heterocycloalkyl, heteroaryl, haloalkyl, haloaryl, haloheteroaryl, alkoxy, aryloxy, alksiliyl, arylsiliyl, heteroarylsiliyl, alkyl (hetero) arylsiliyl, alkenyl, cycloalkenyl, alkynyl, hydroxyl, mercapto, nitro, cyano, amino, mono- or dialkylamino, mono- or diarylamino, ester, isonitrile, alkoxycarbonyl, amido, alkoxycarbonylamino, aryloxycarbonylamino, sulfonylamino, sulfamoyl, carbamoyl, alkylthio, sulfinyl, ureido, phosphonamido, imino, sulfo, carboxyl, hydrazino, or combinations thereof; two or more adjacent R⁷ and R⁸ can selectively connect to form a fused ring; any two groups in R⁷ and R⁸ can be connected to form a cyclic system; among them, alkyl(hetero)arylsilyl is alkarylsilyl or alkyl hetero arylsilyl.

7. The helically chiral platinum (II) and palladium (II) complex circularly polarized luminescent material according to claim 1, wherein its structure is a P-type isomer as shown in the following figures, where M=Pt or Pd, and its M-type enantiomer:

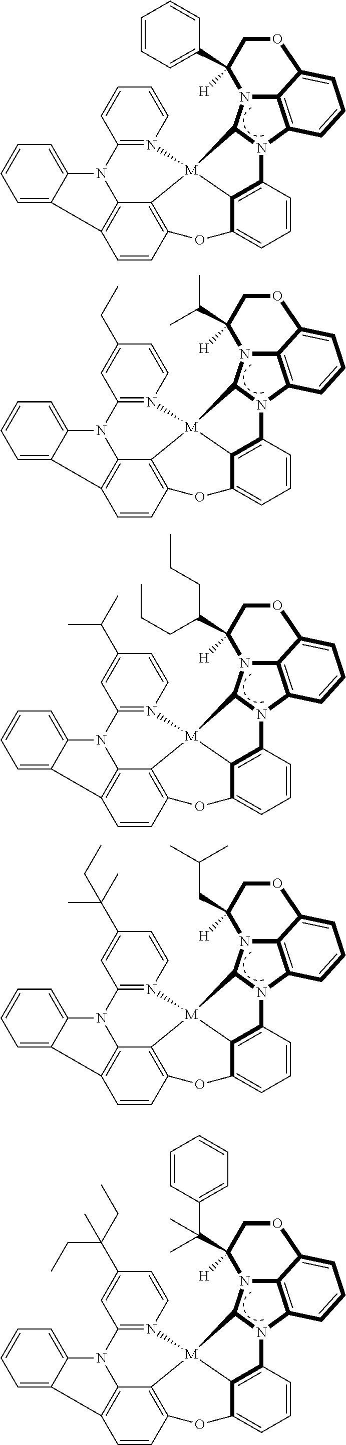

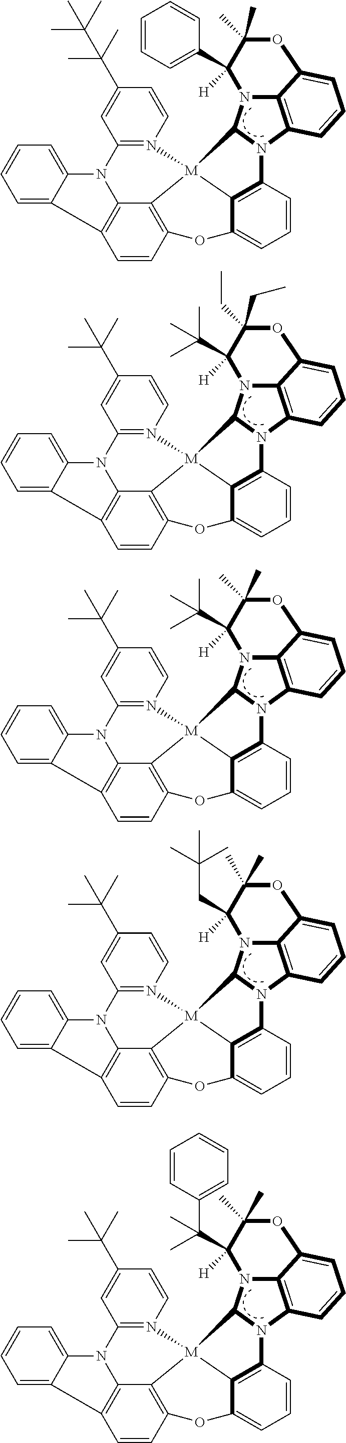

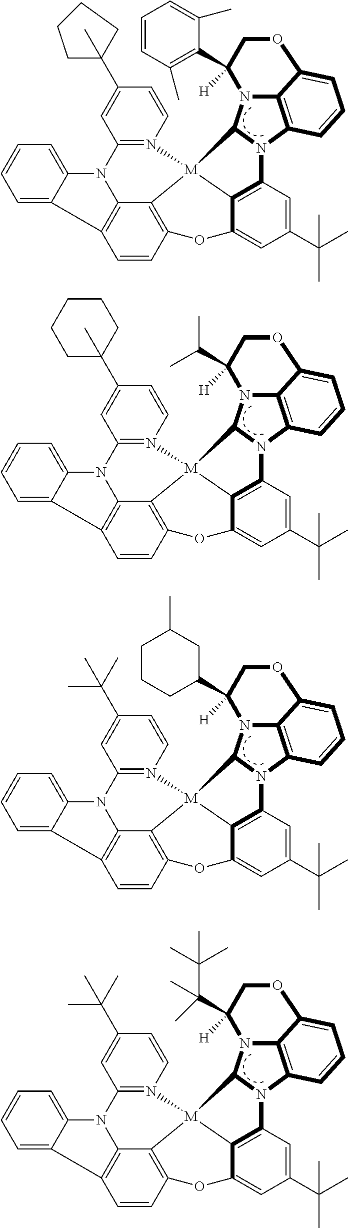

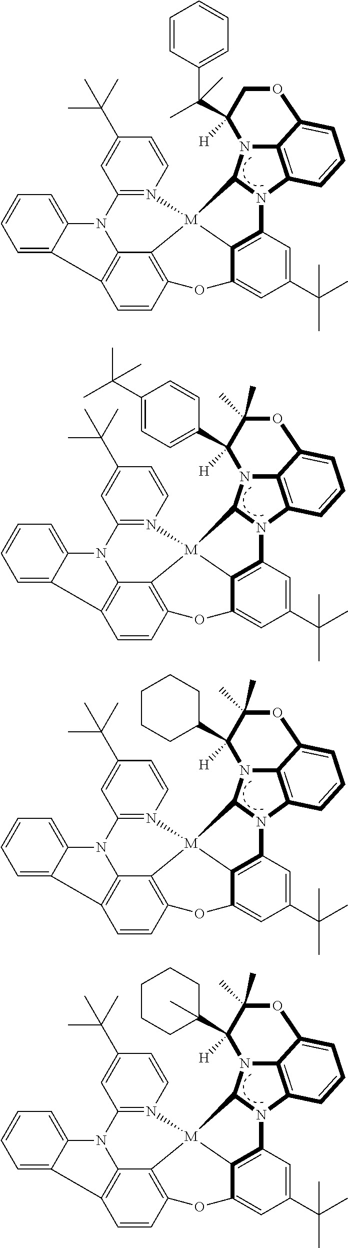

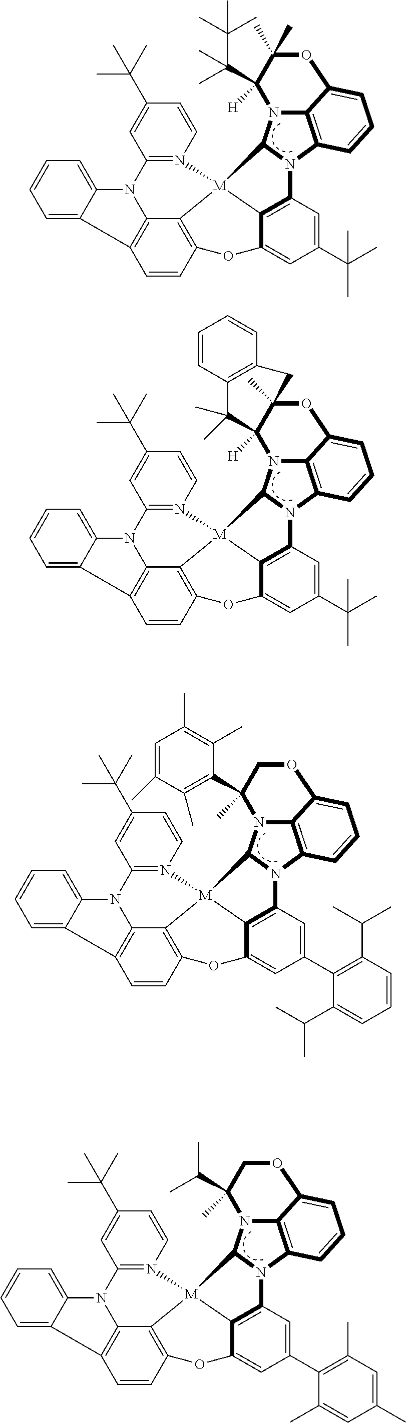

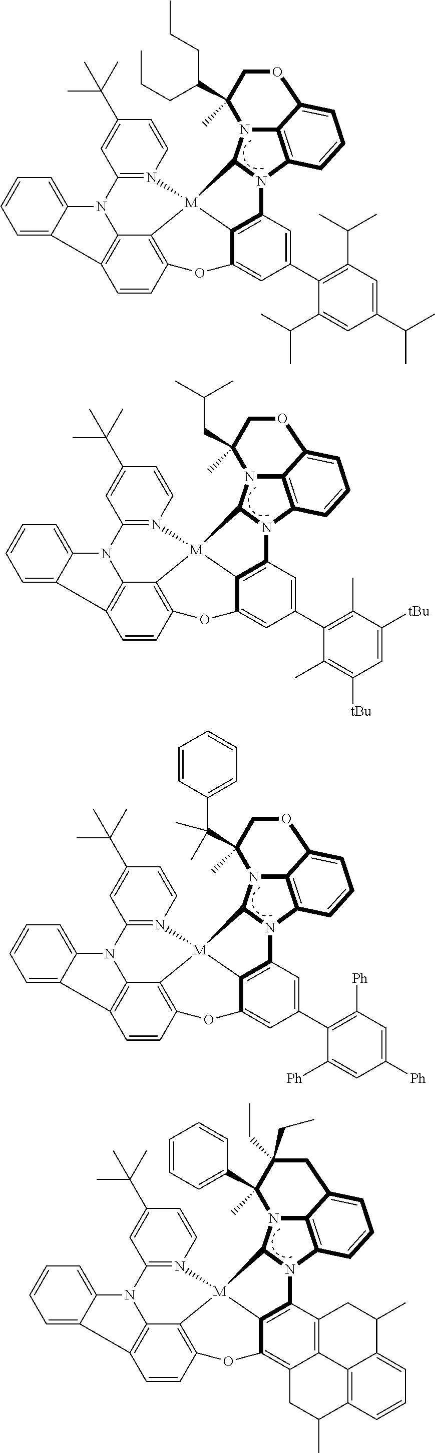

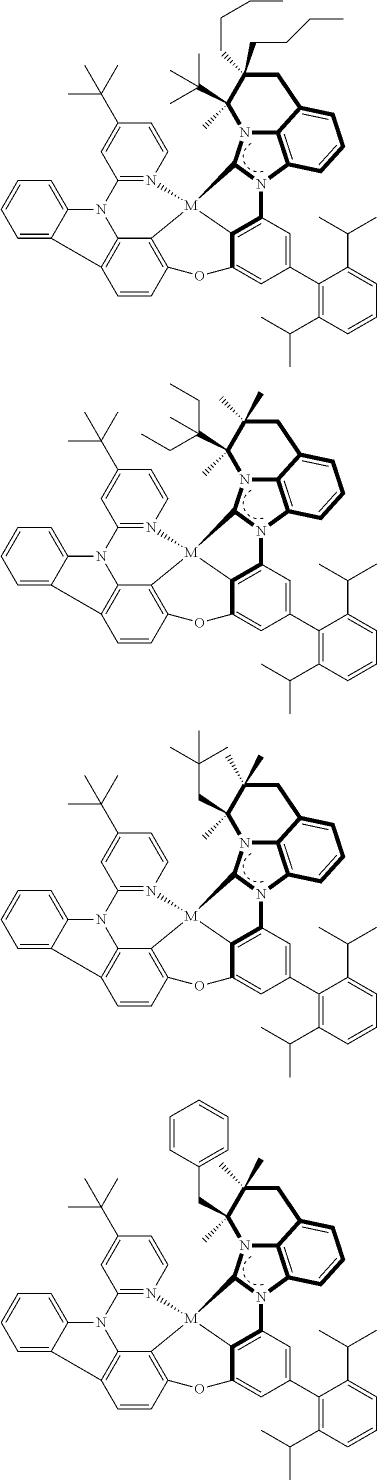

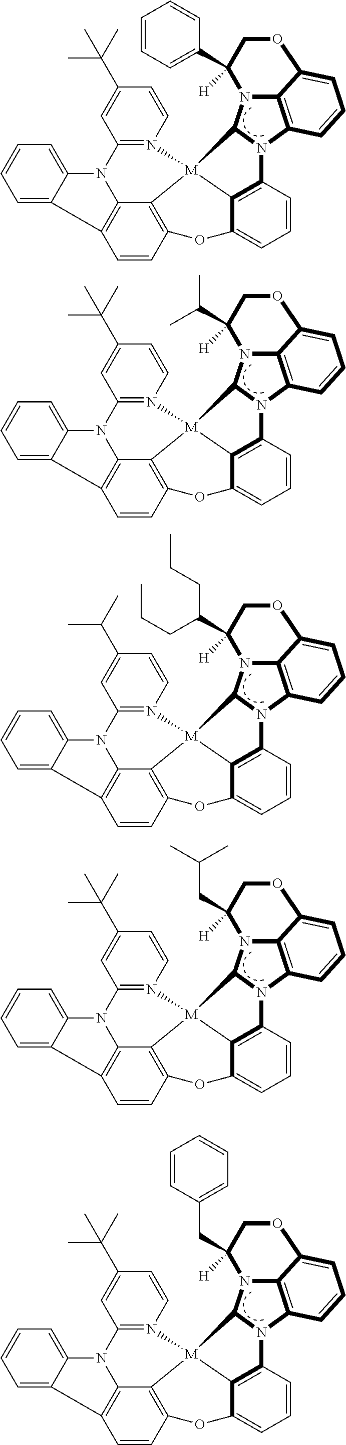

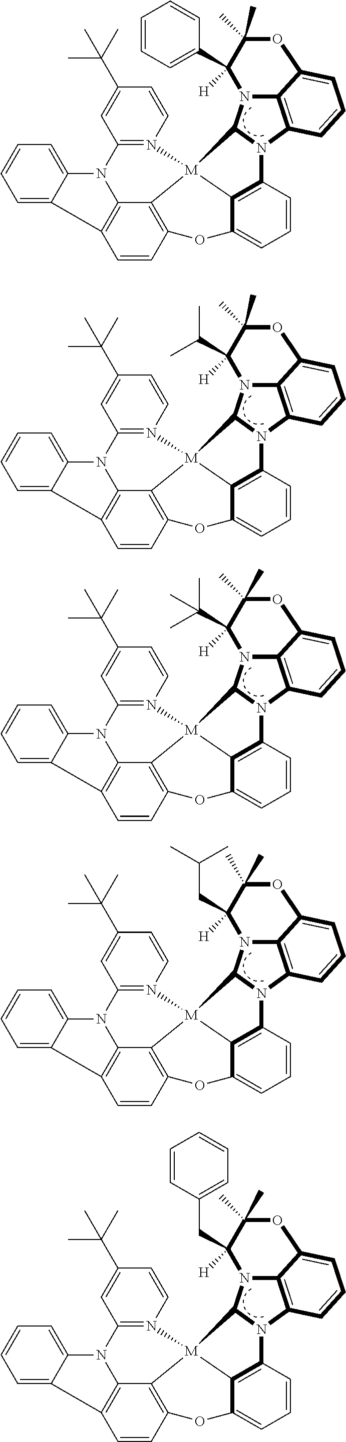

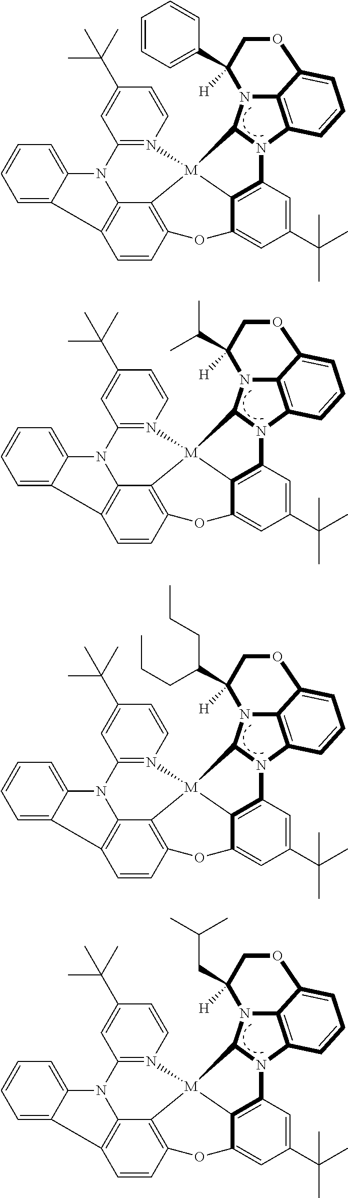

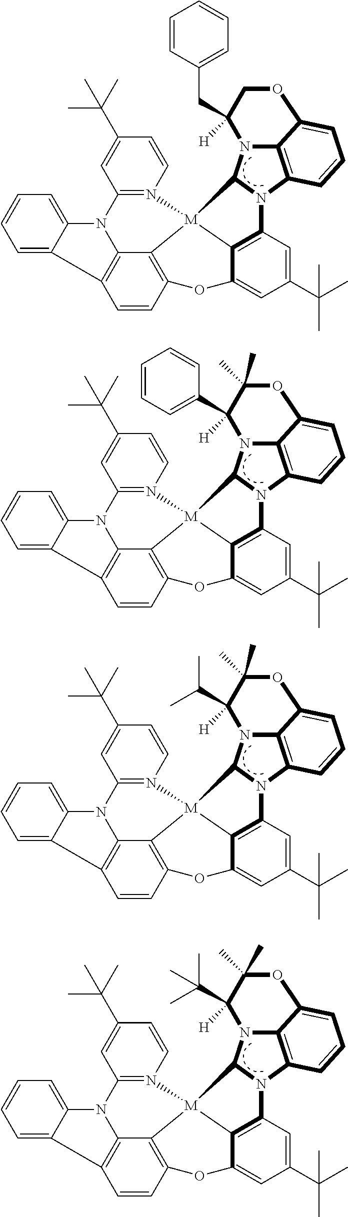

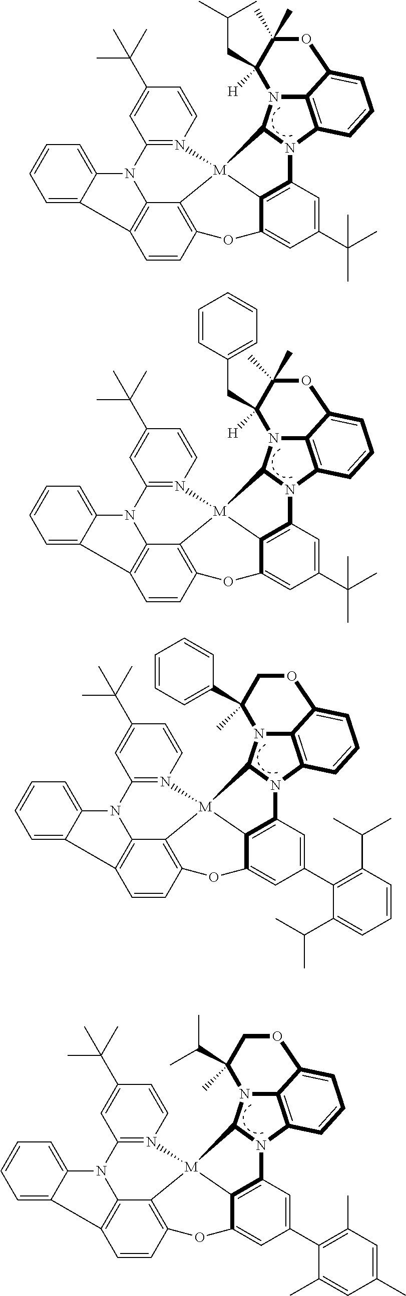

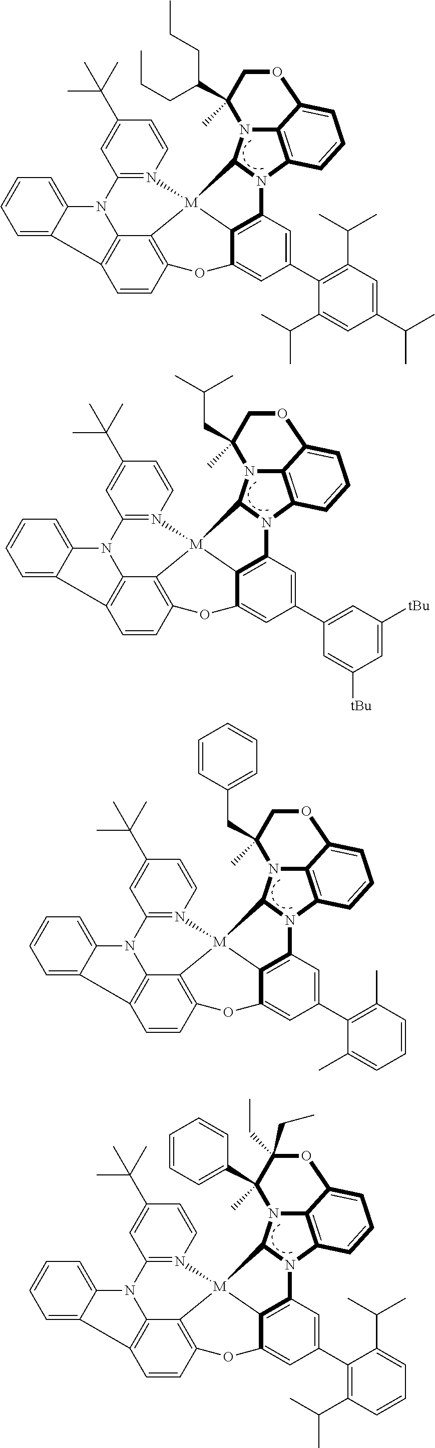

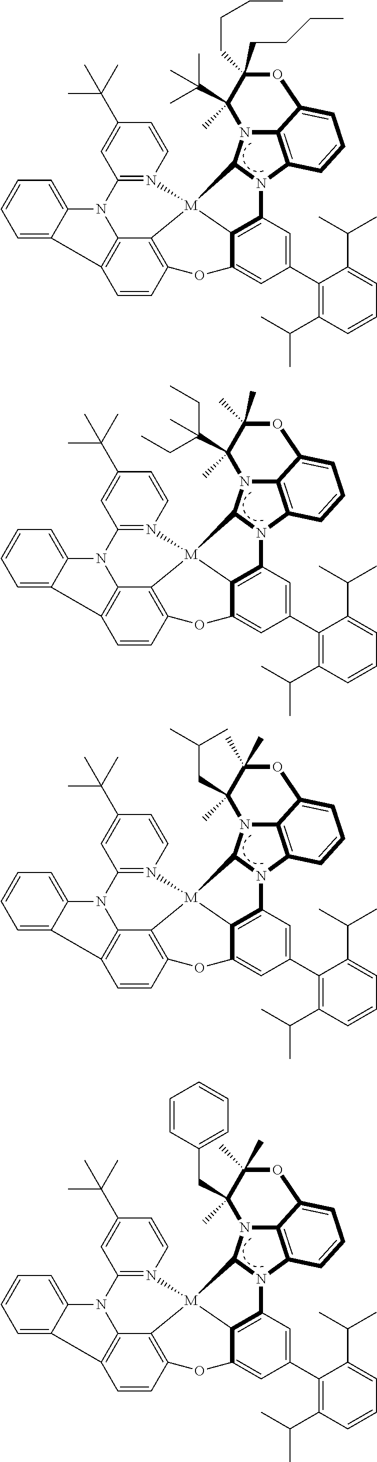

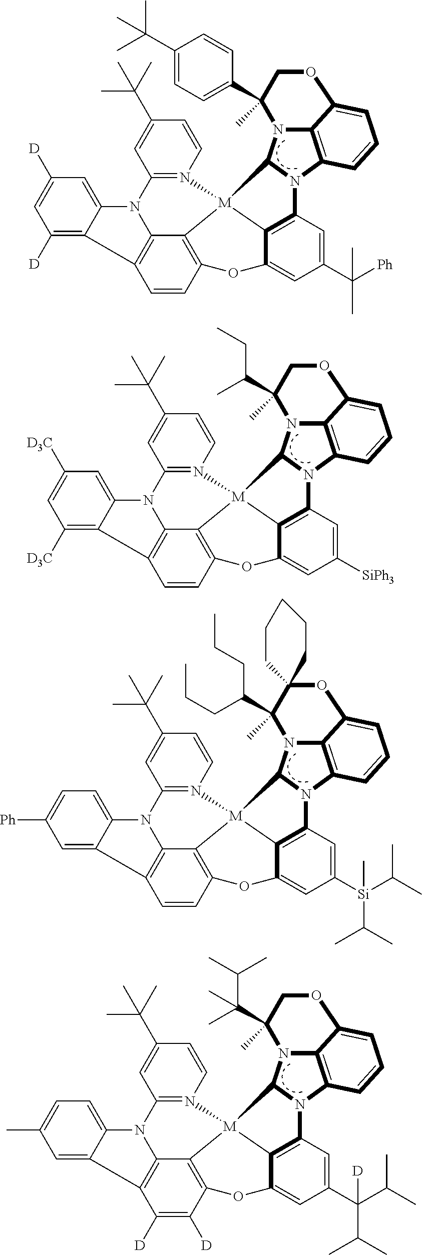

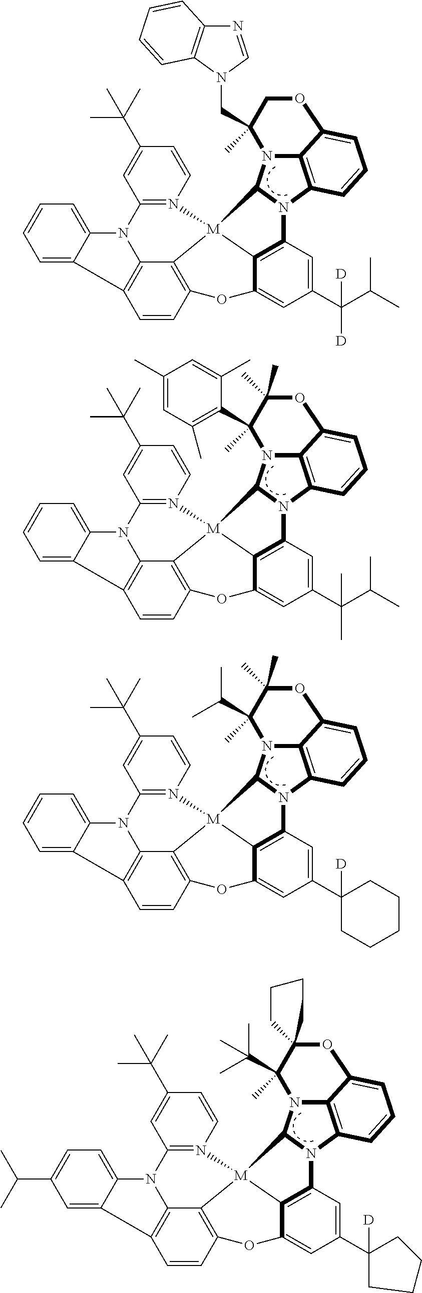

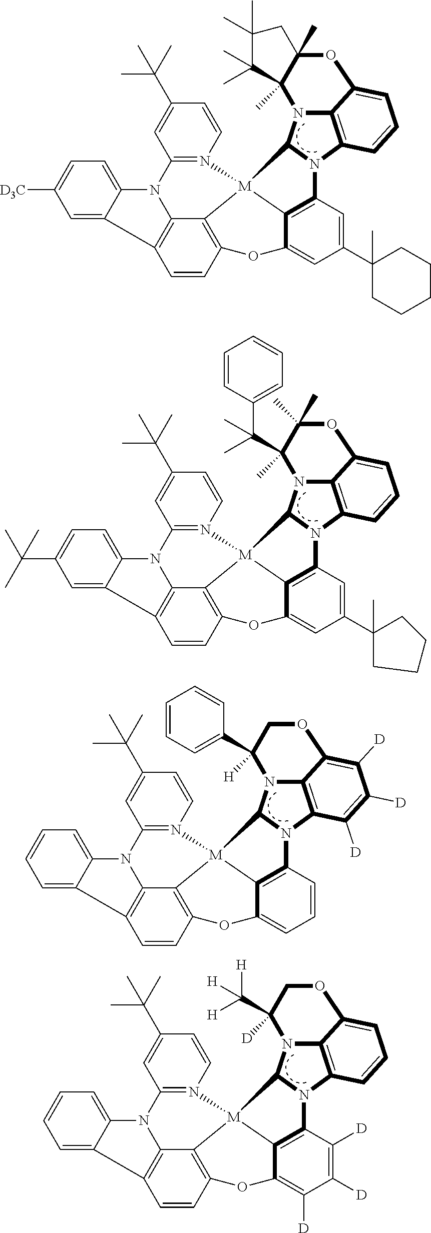

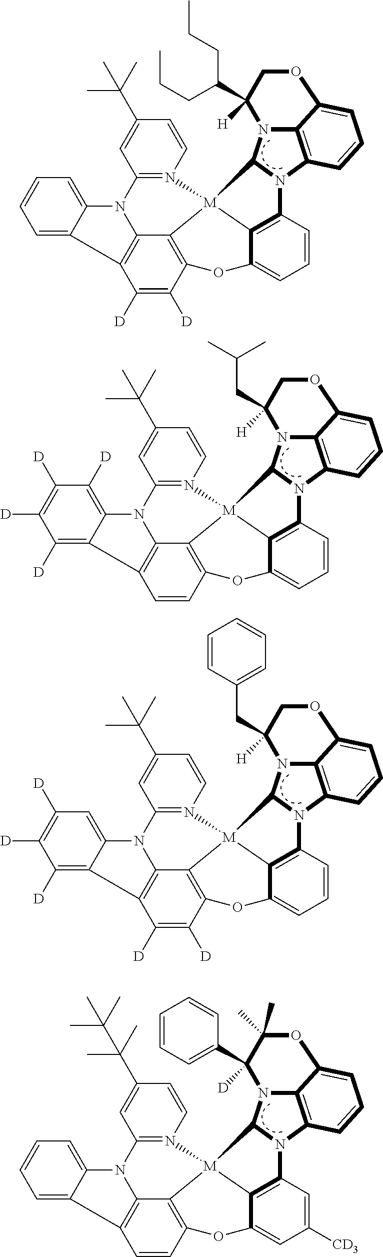

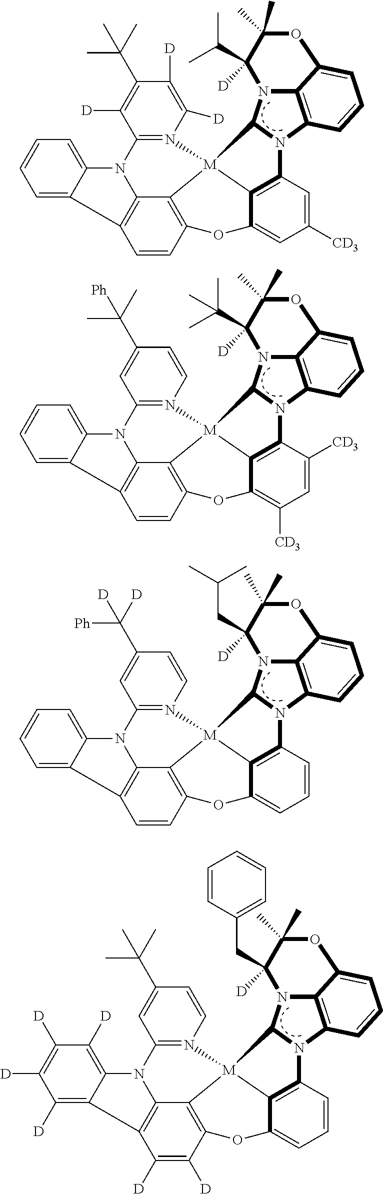

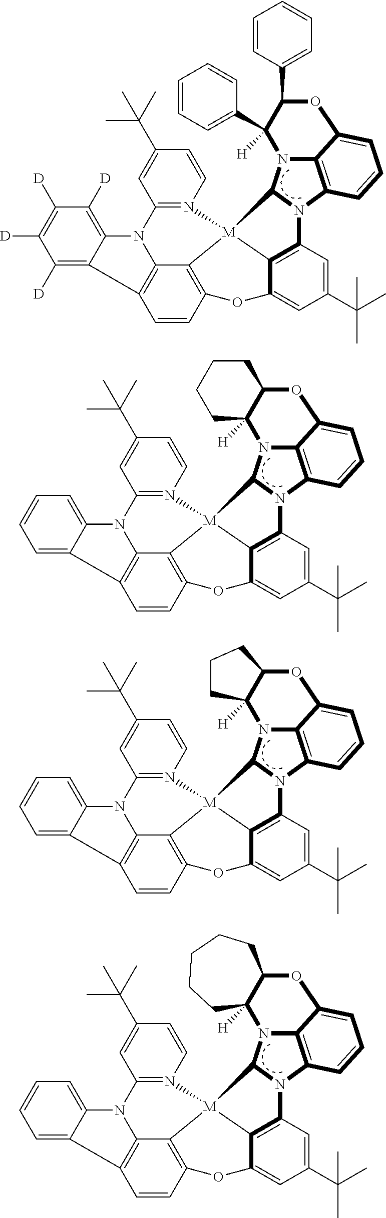

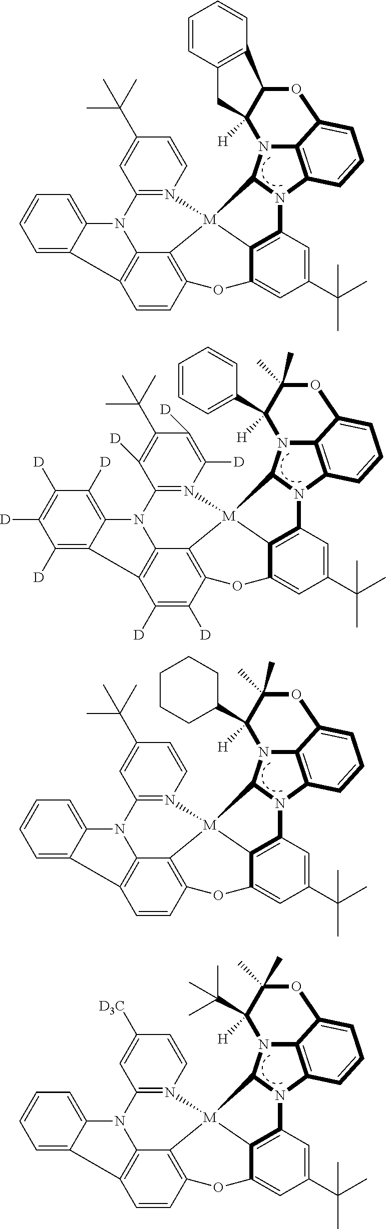

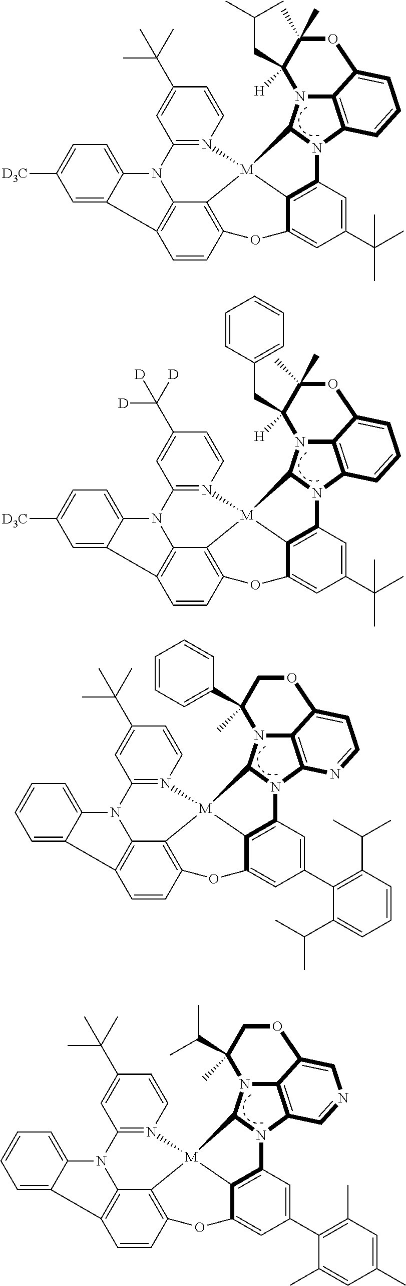

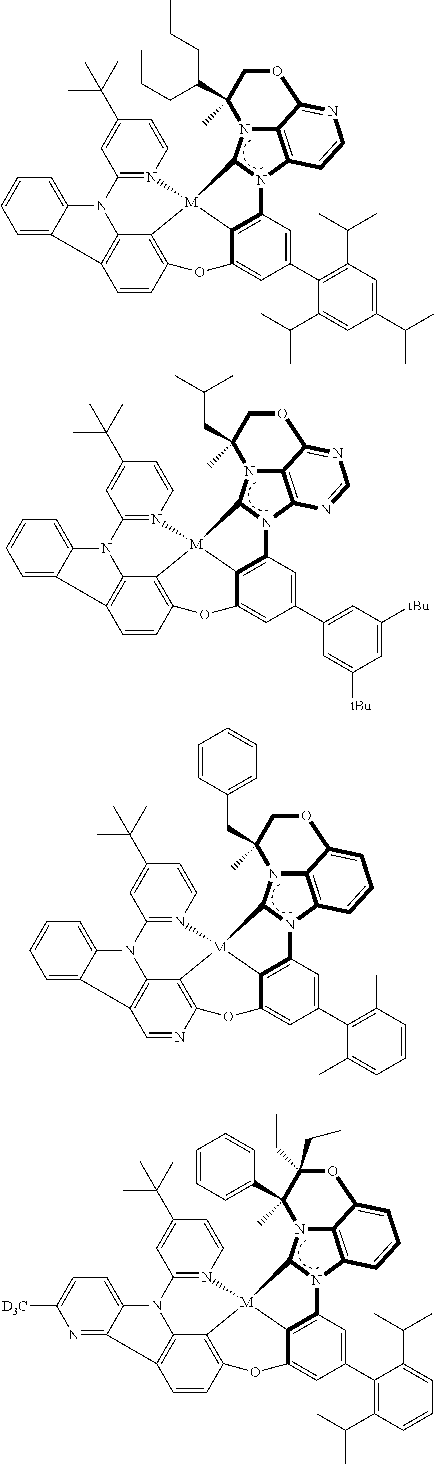

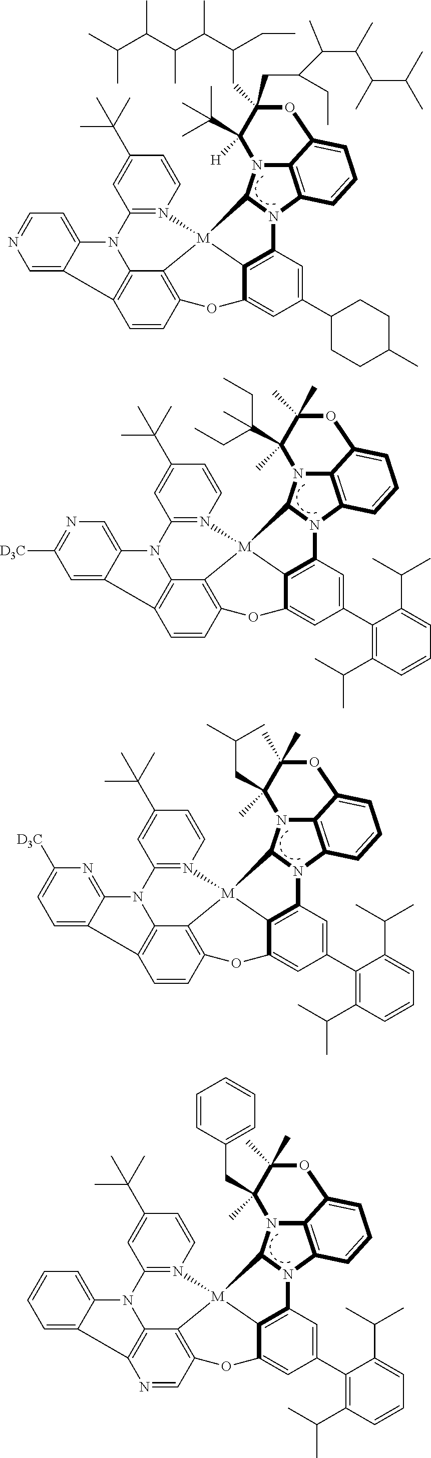

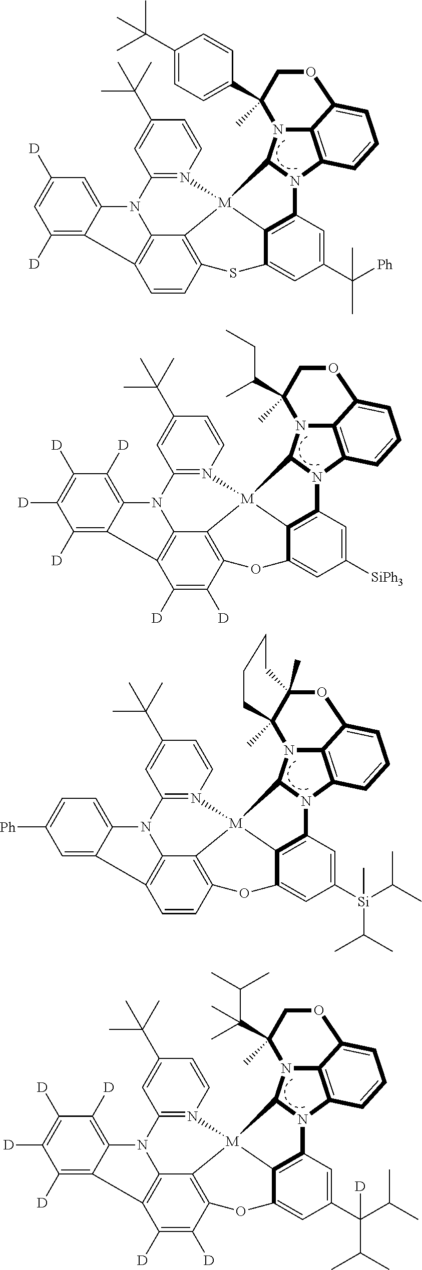

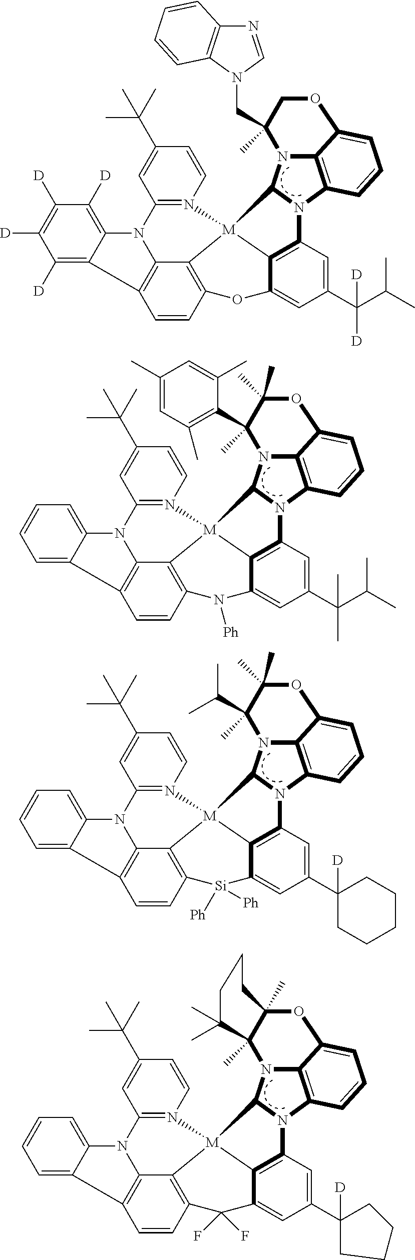

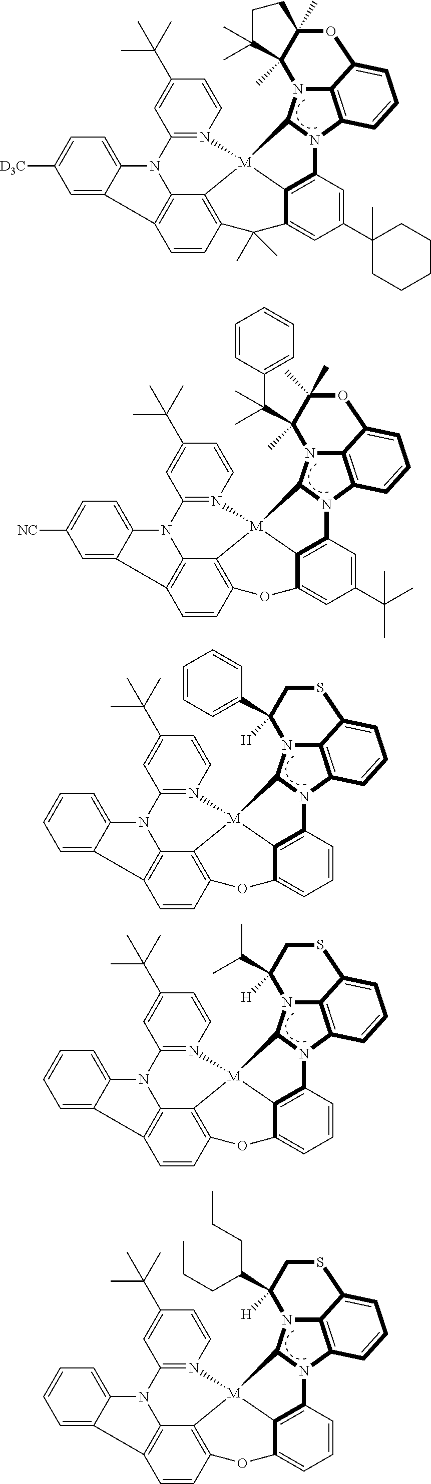

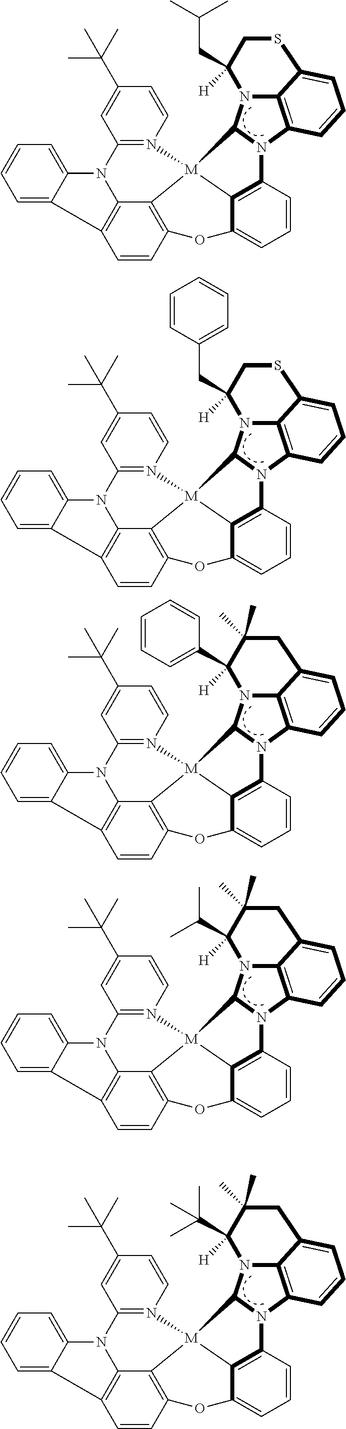

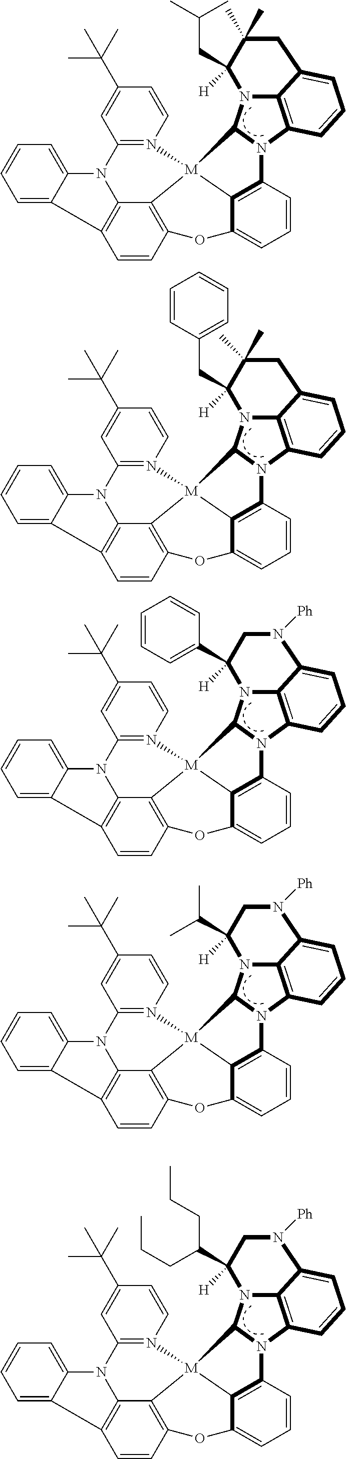

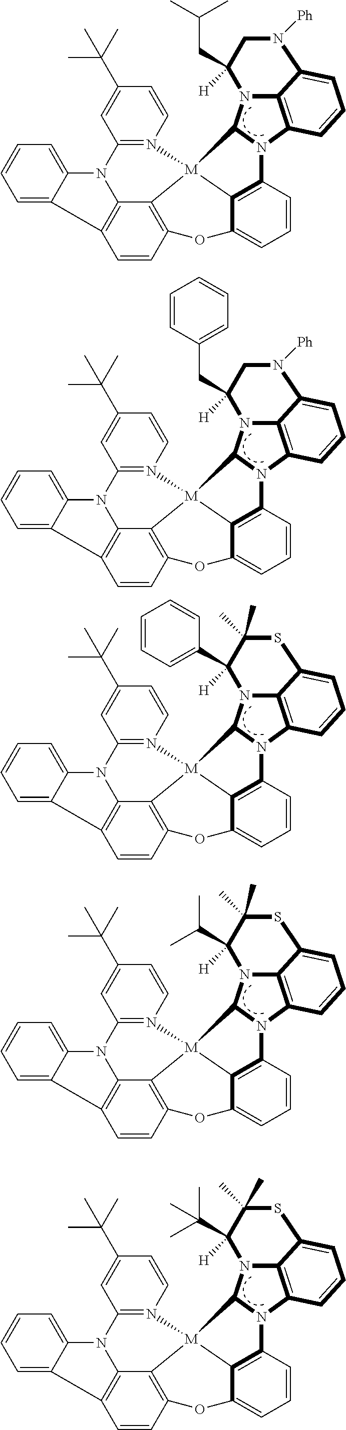

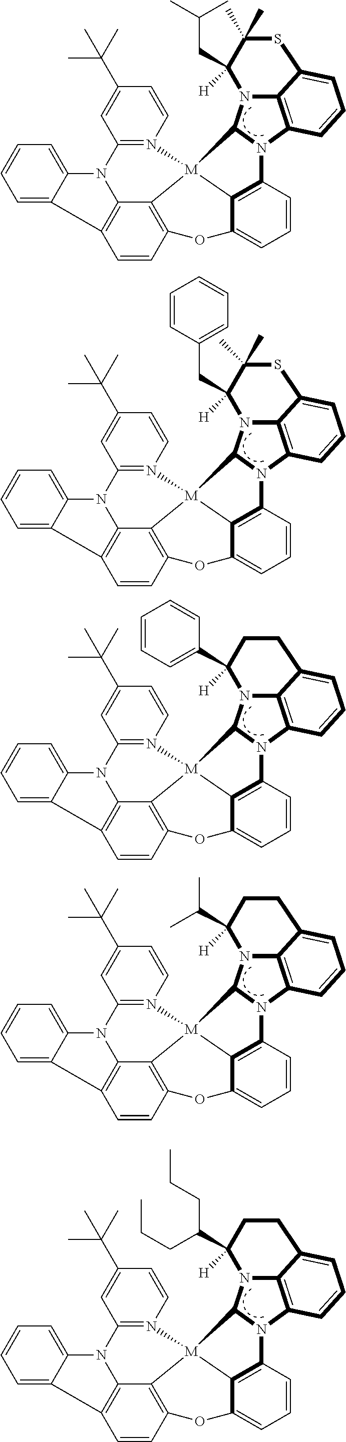

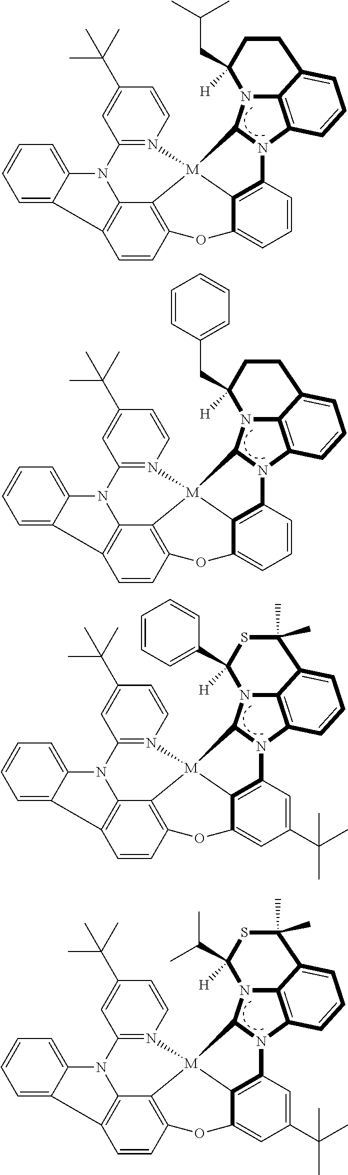

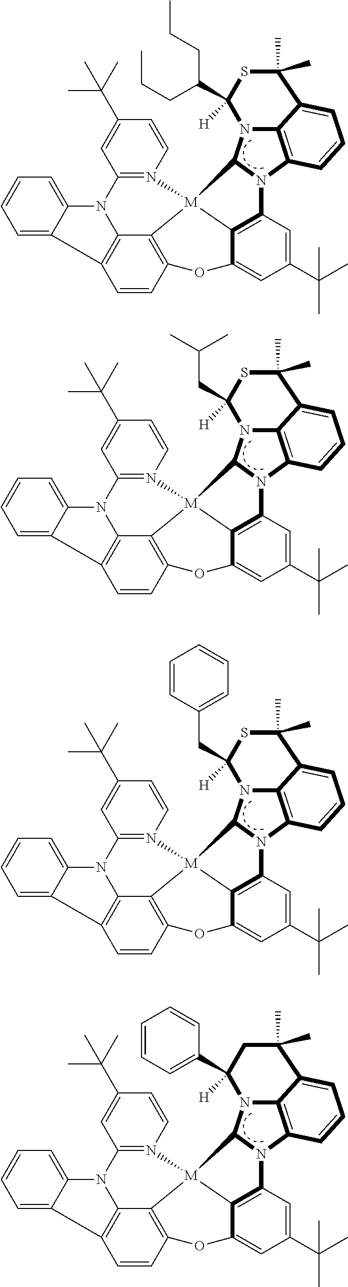

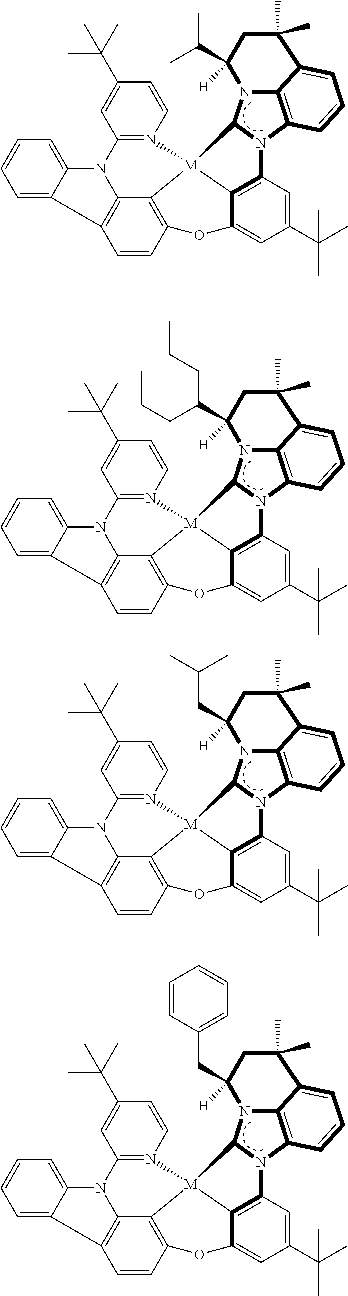

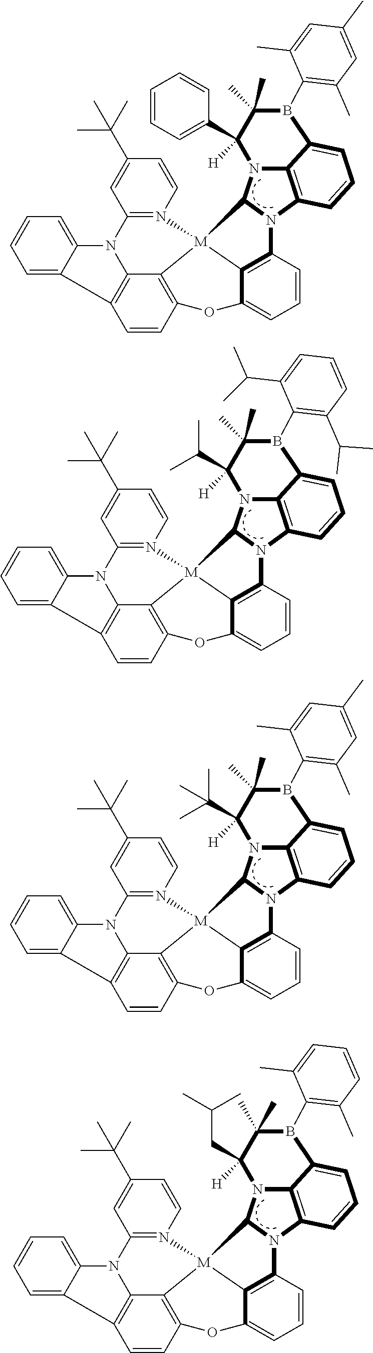

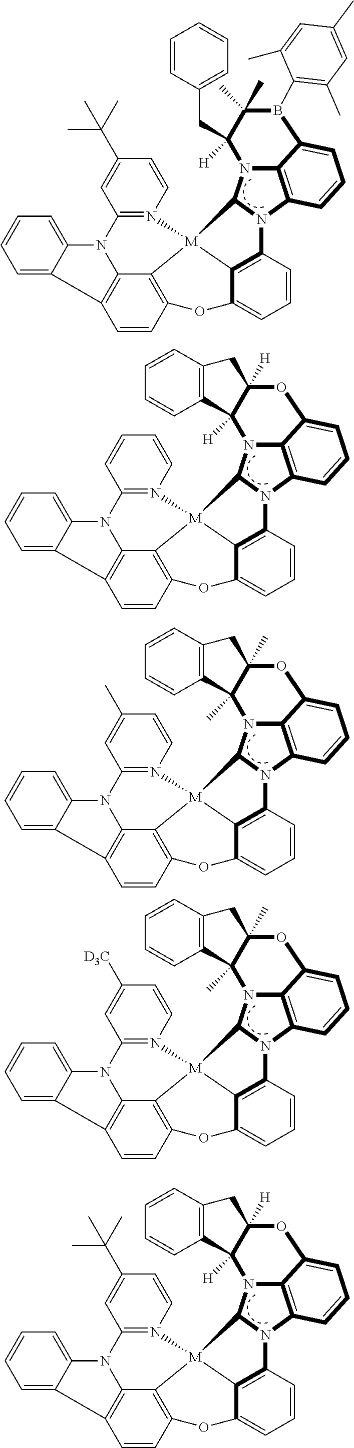

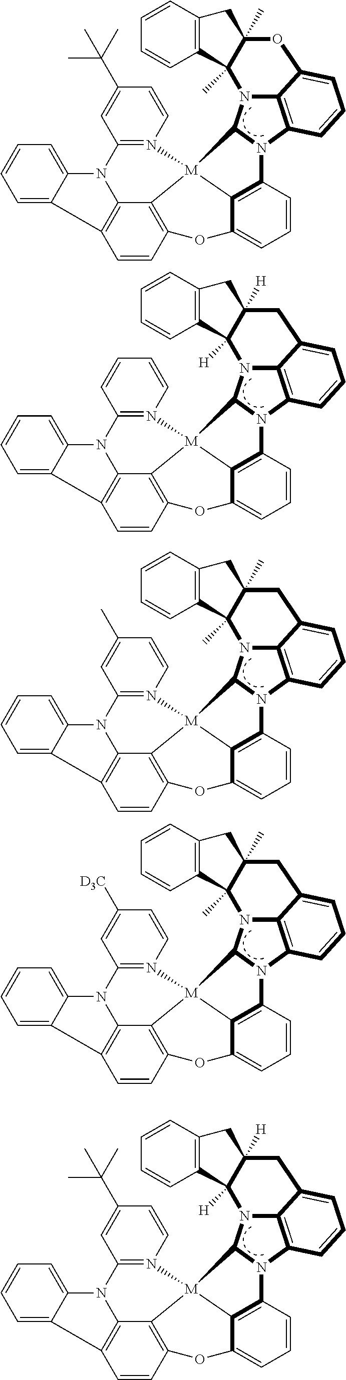

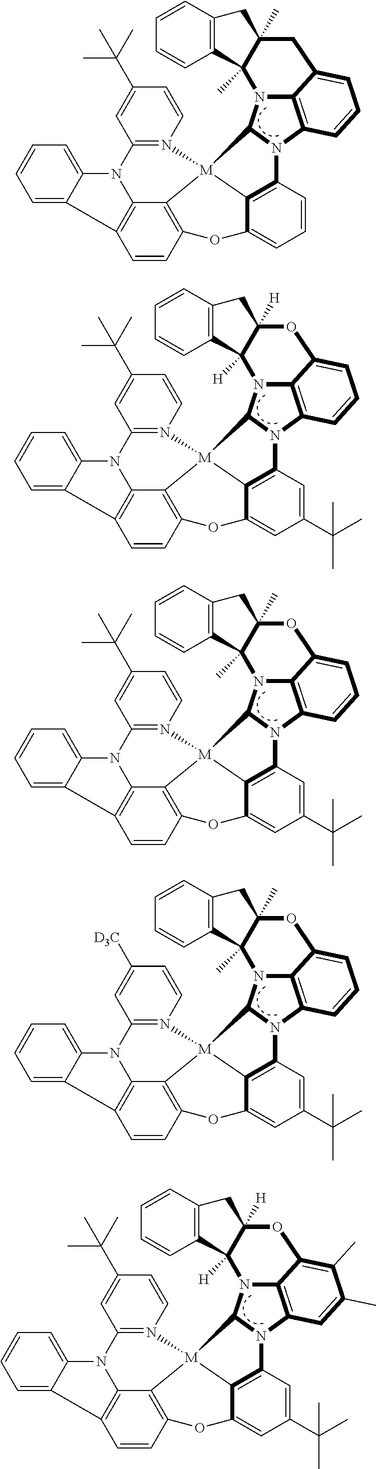

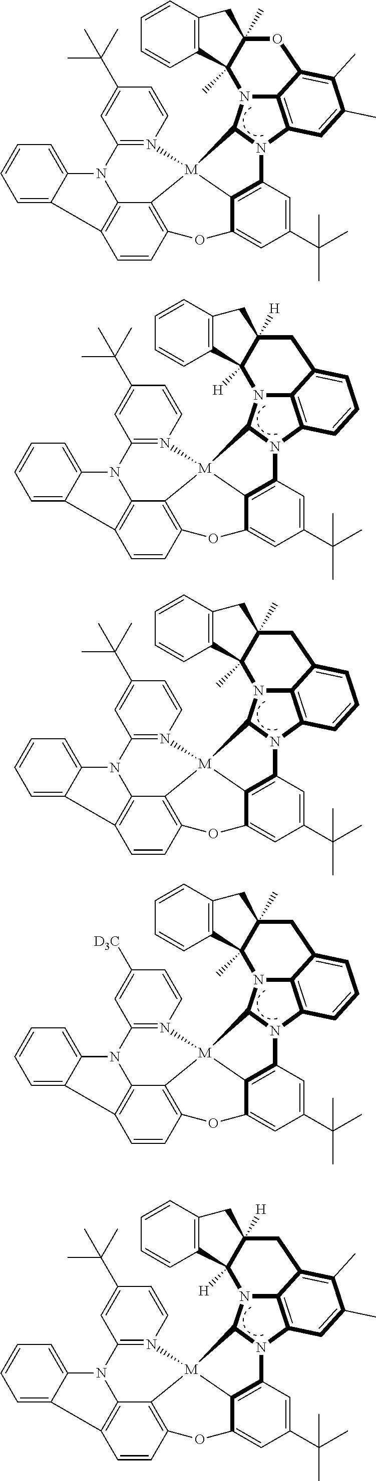

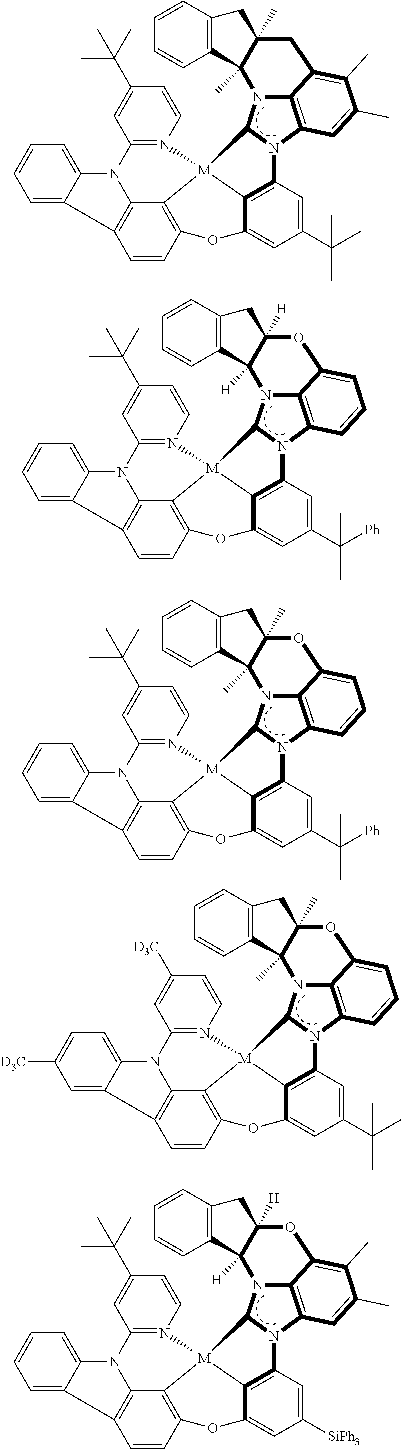

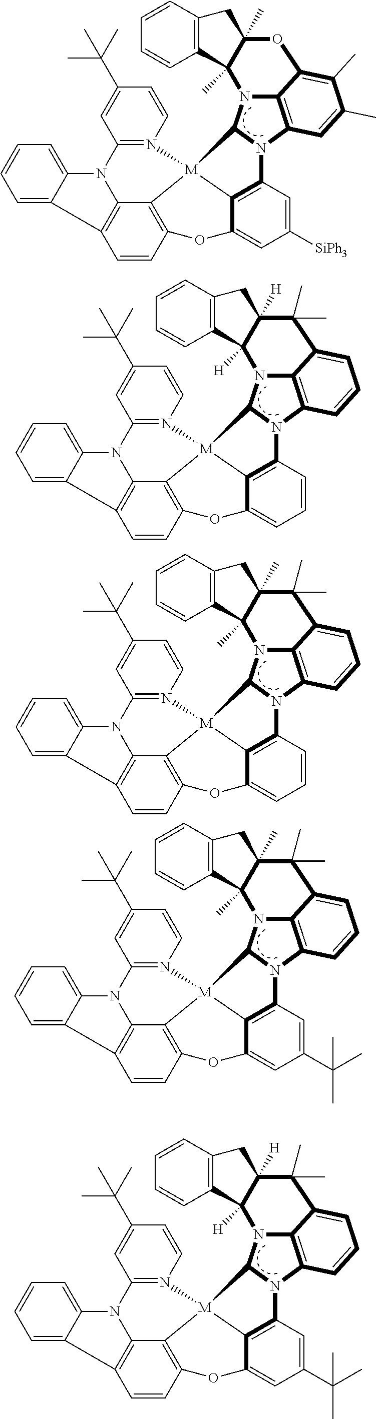

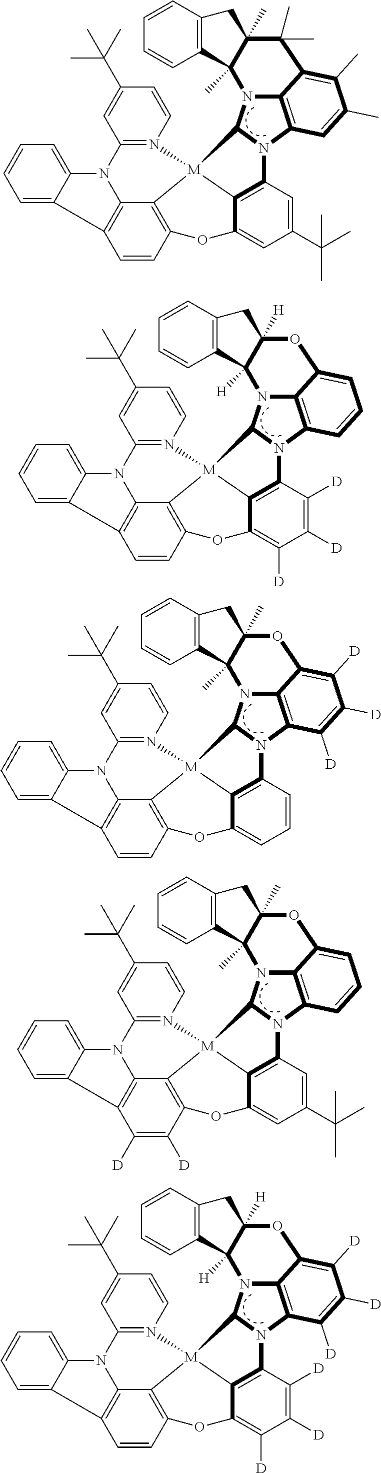

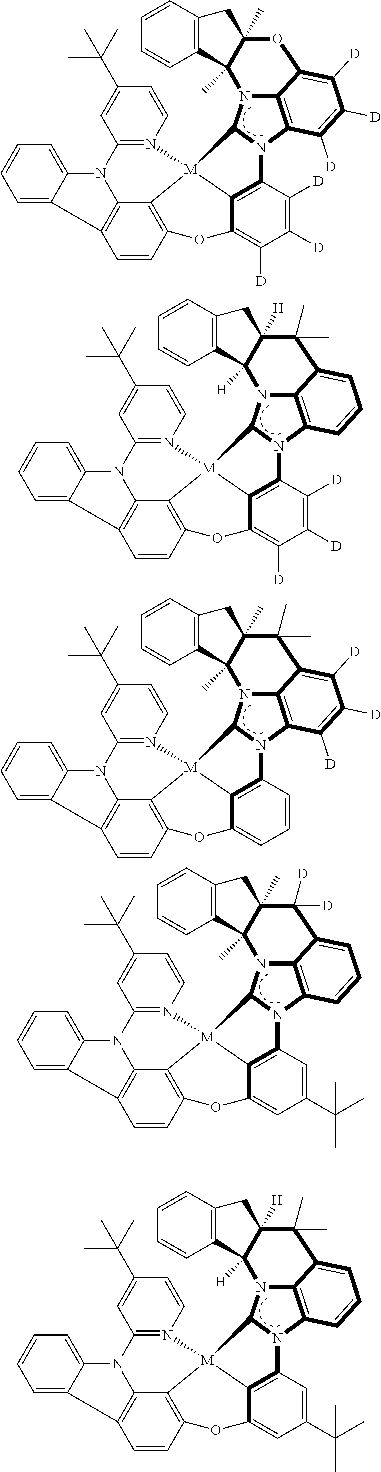

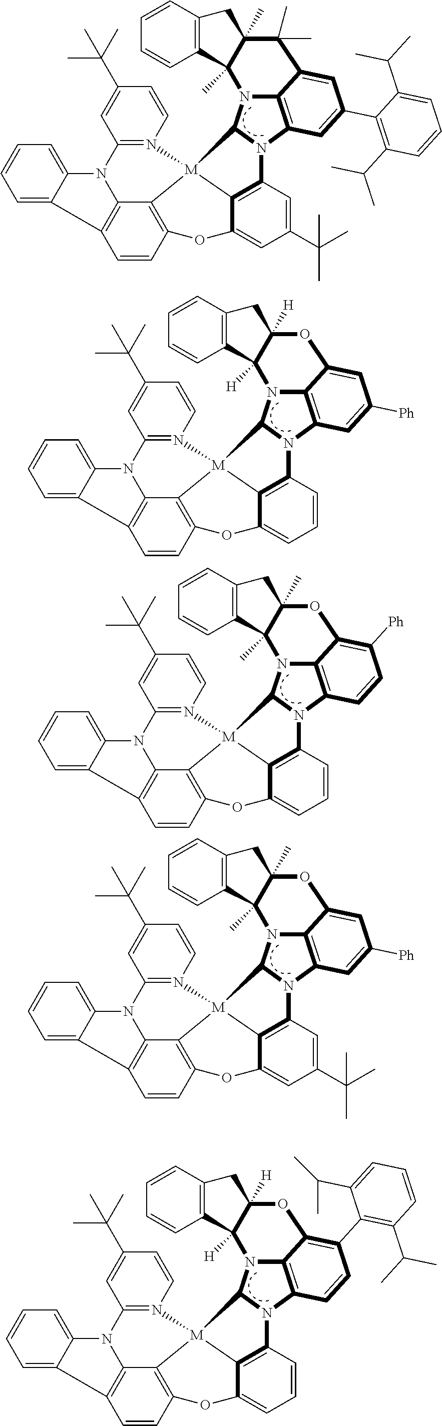

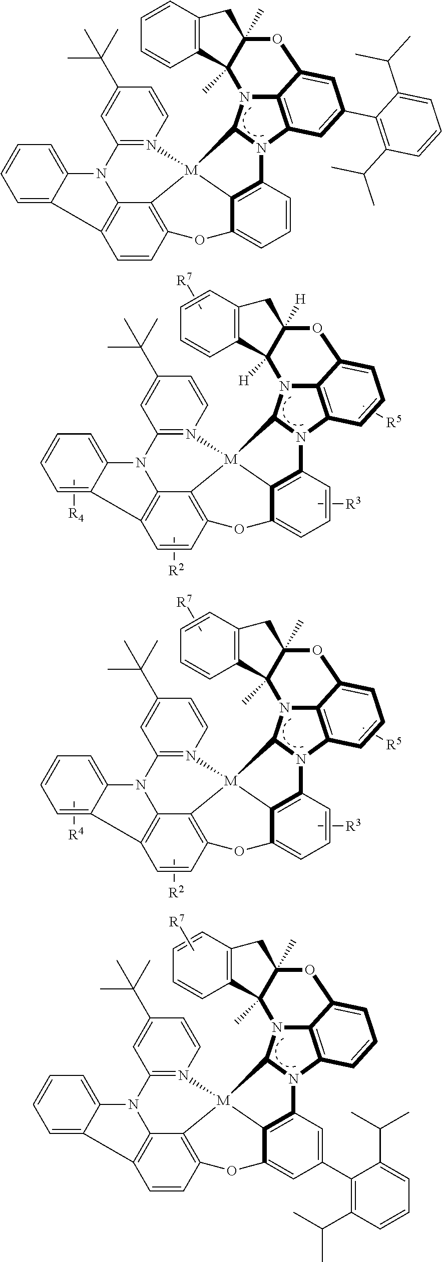

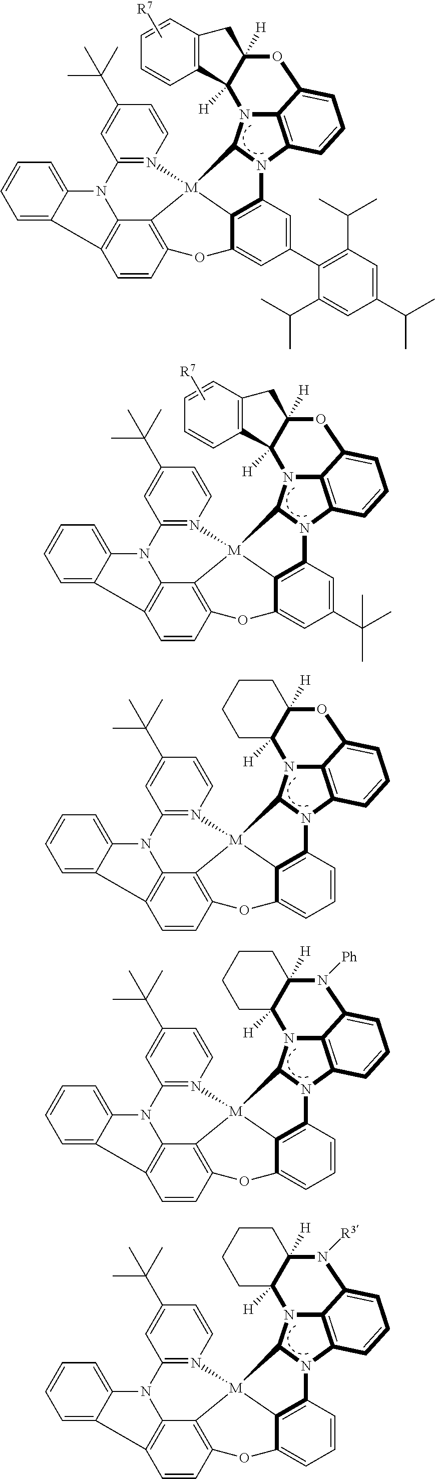

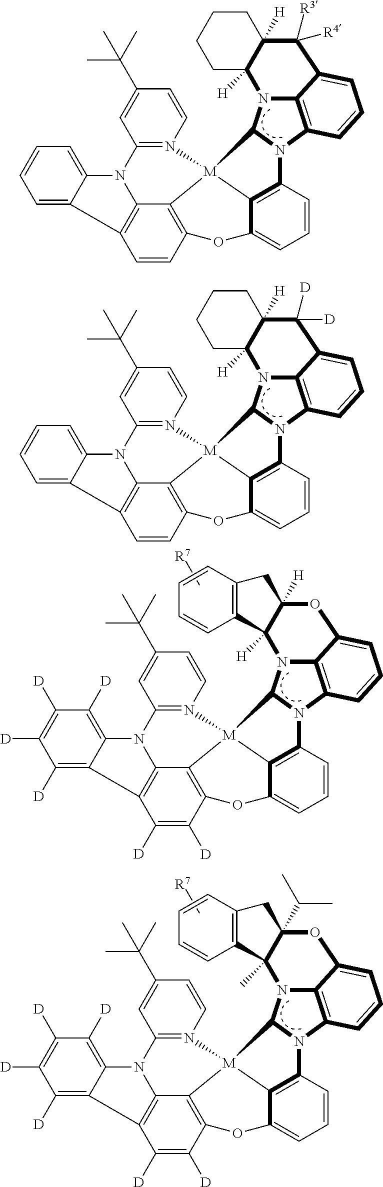

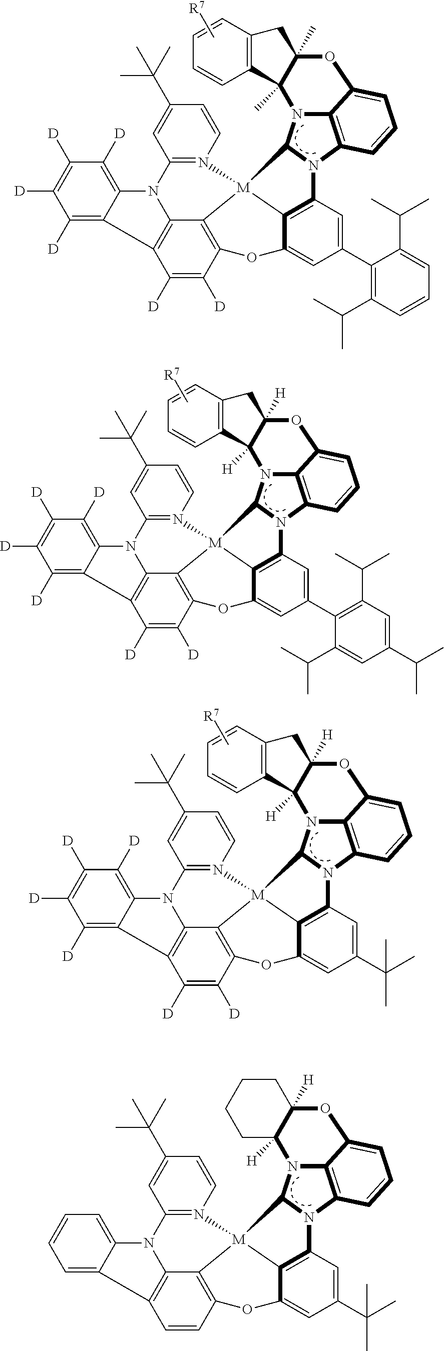

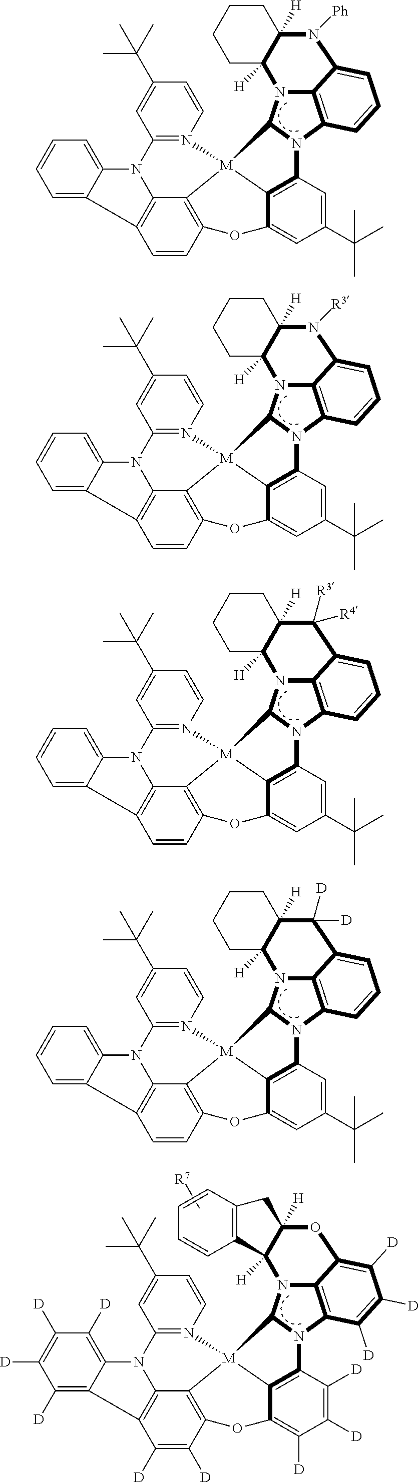

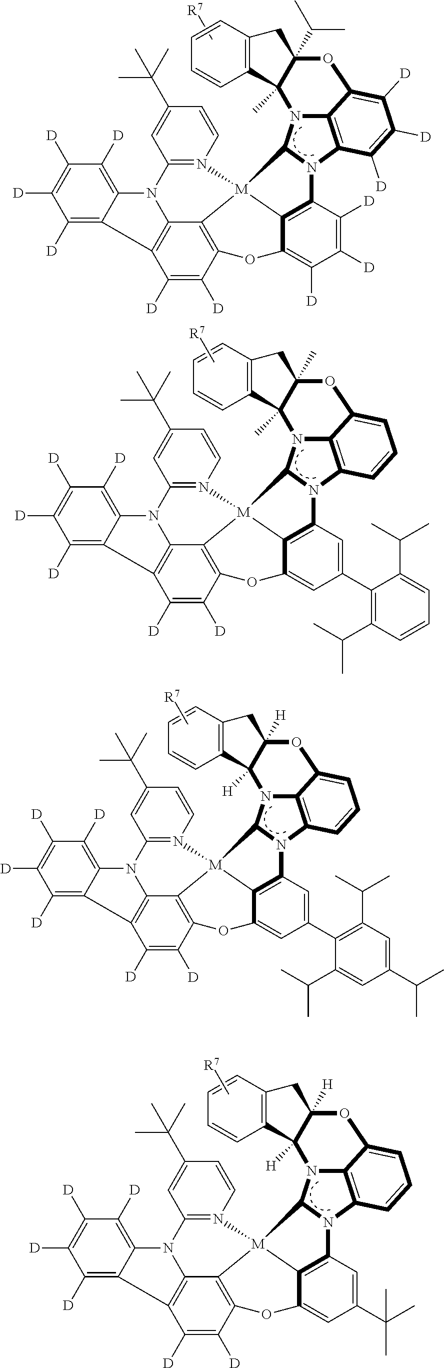

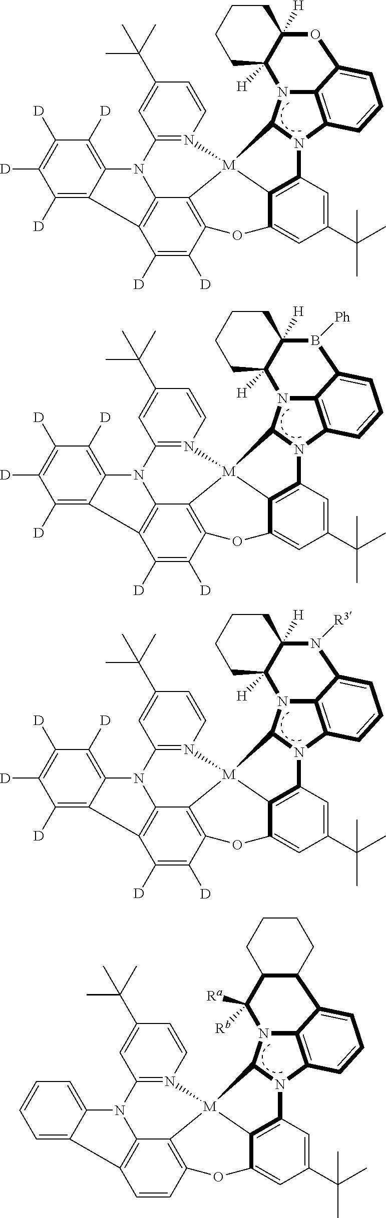

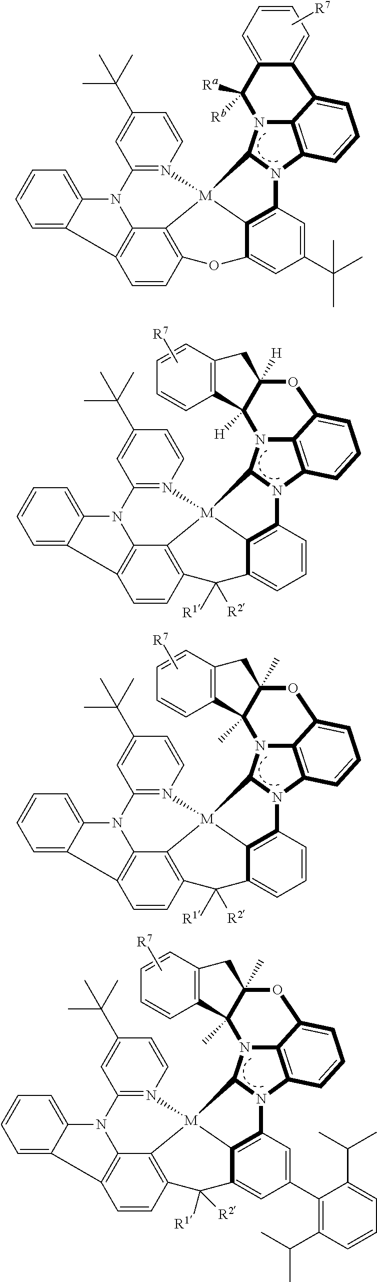

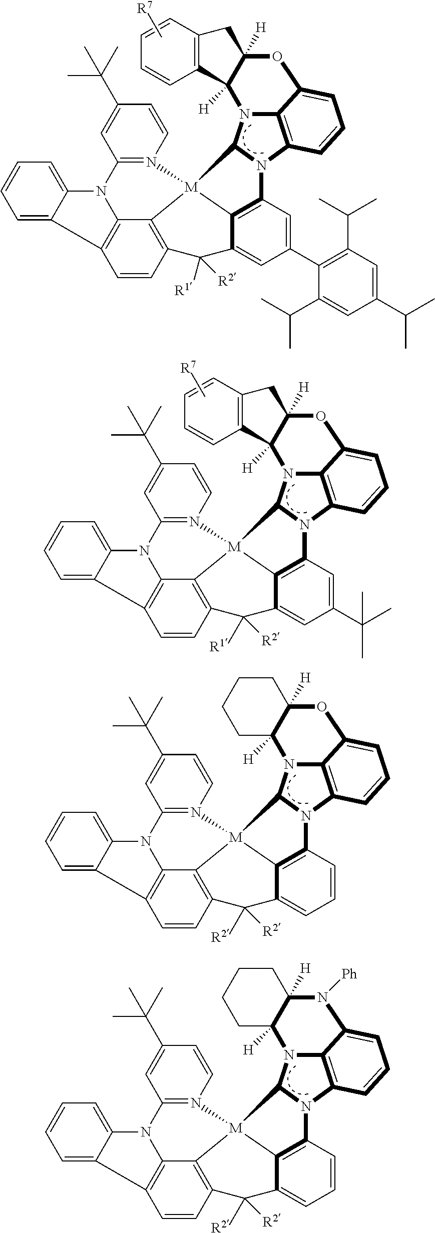

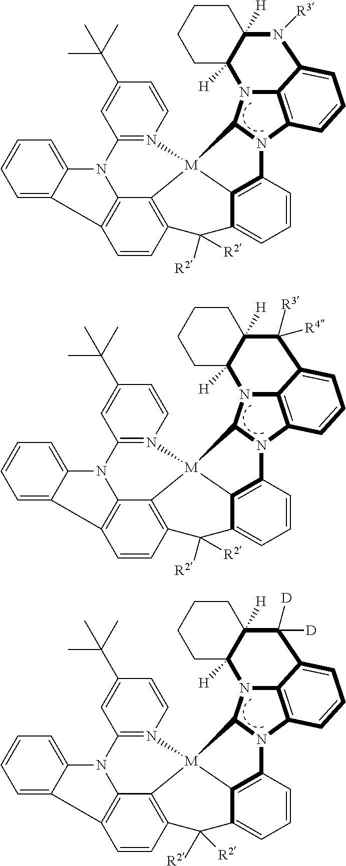

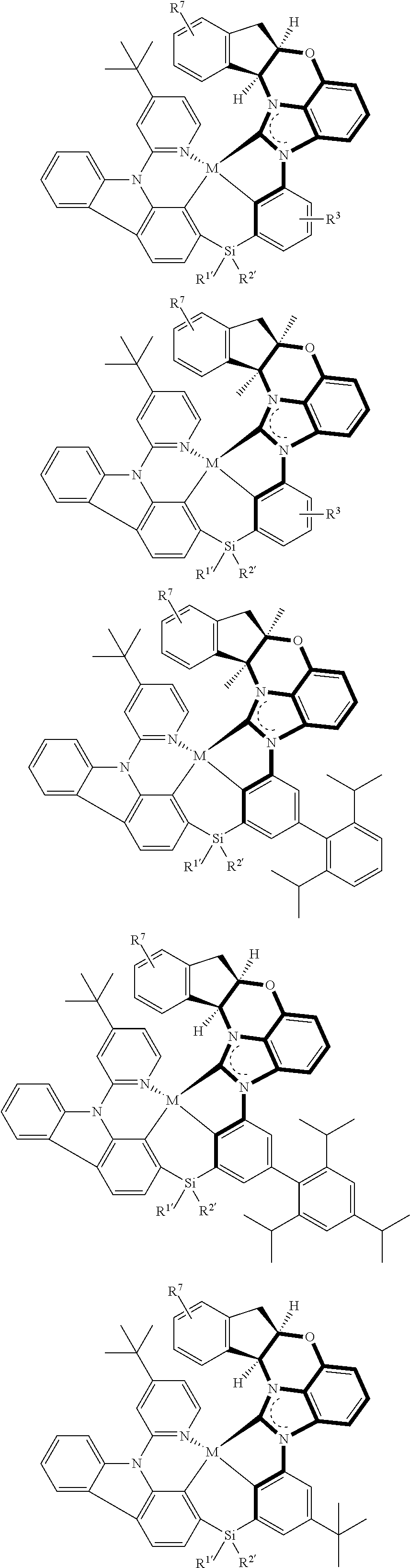

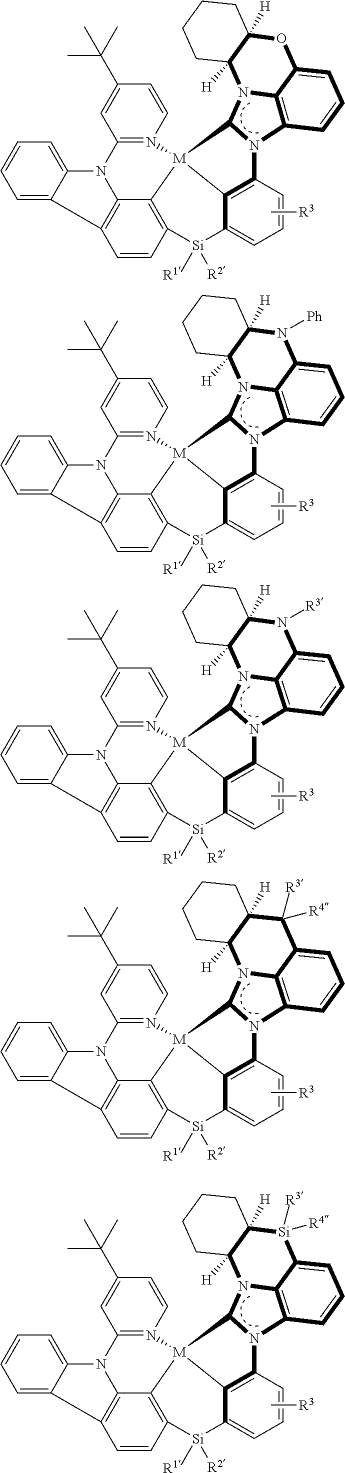

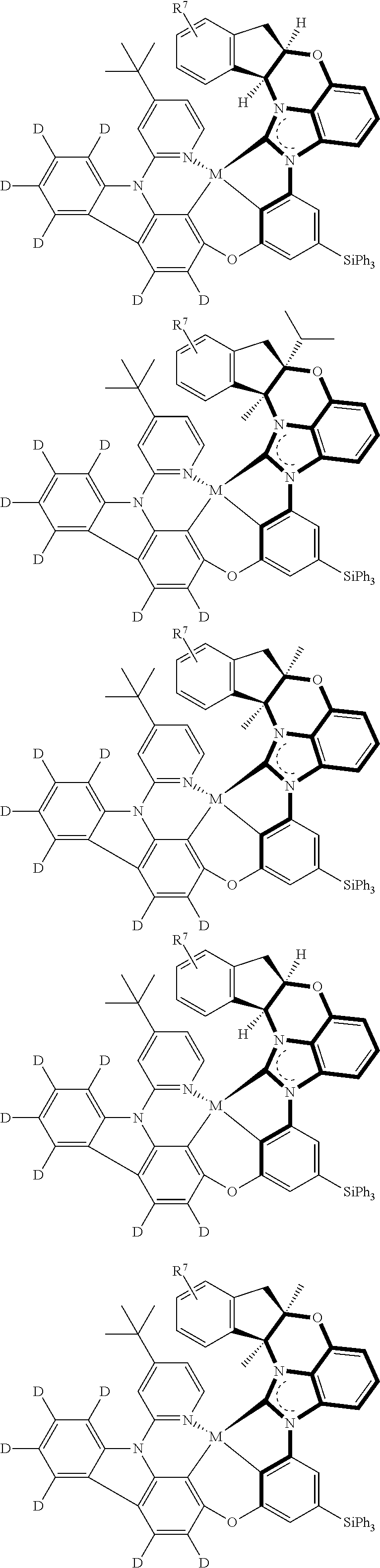

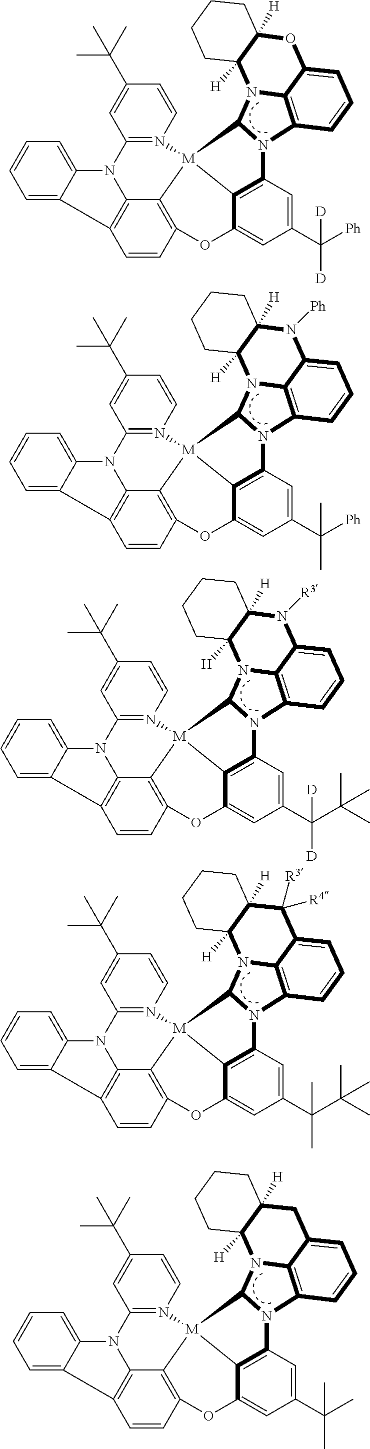

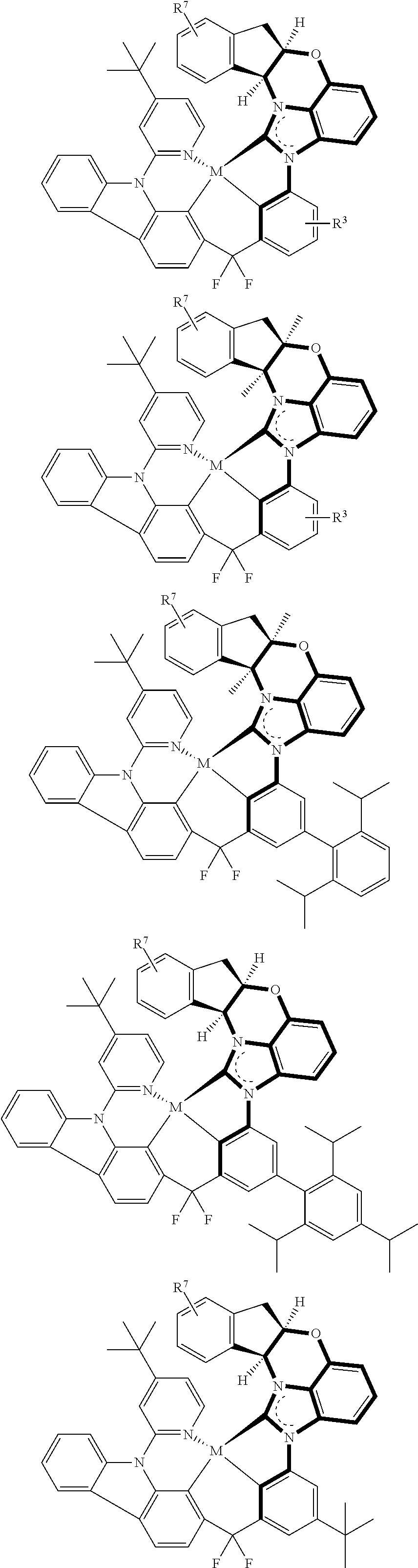

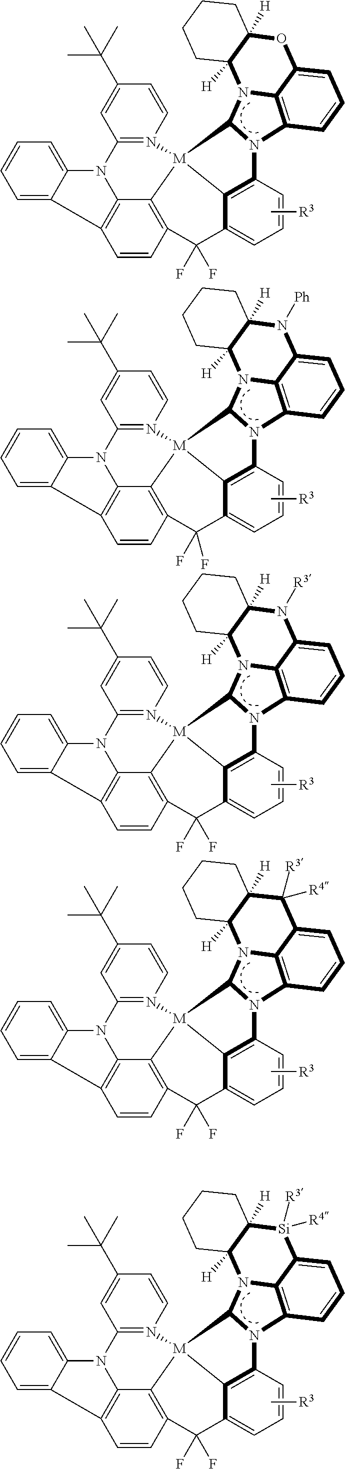

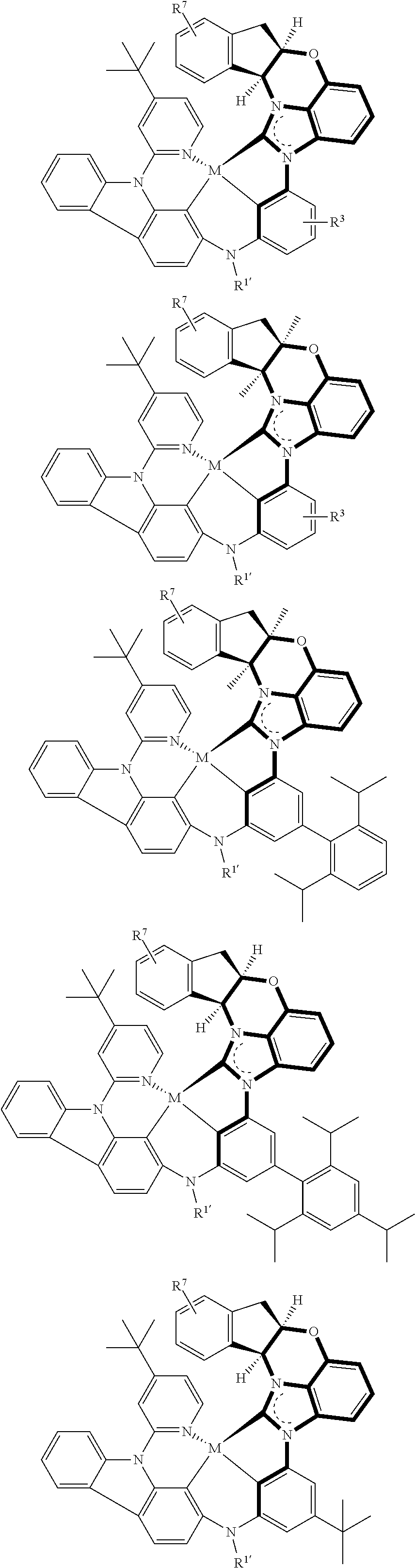

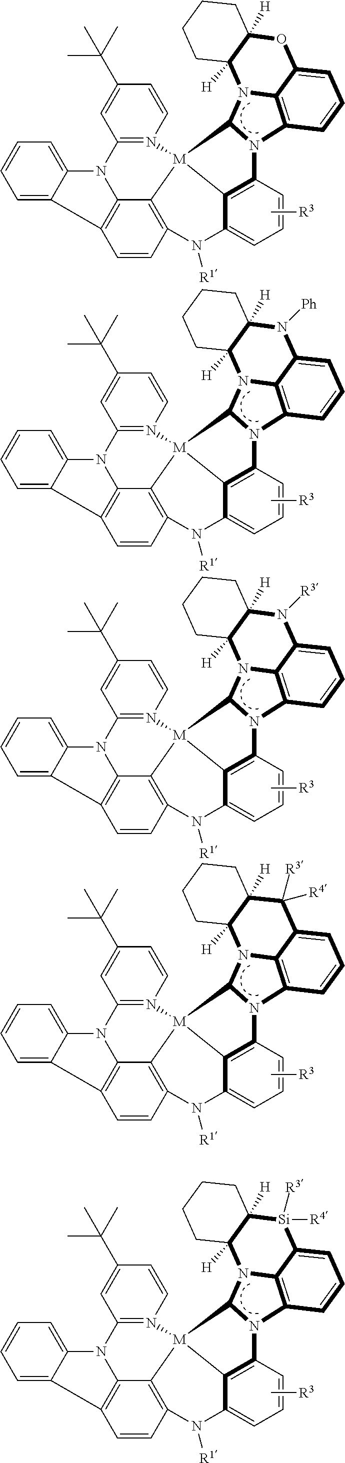

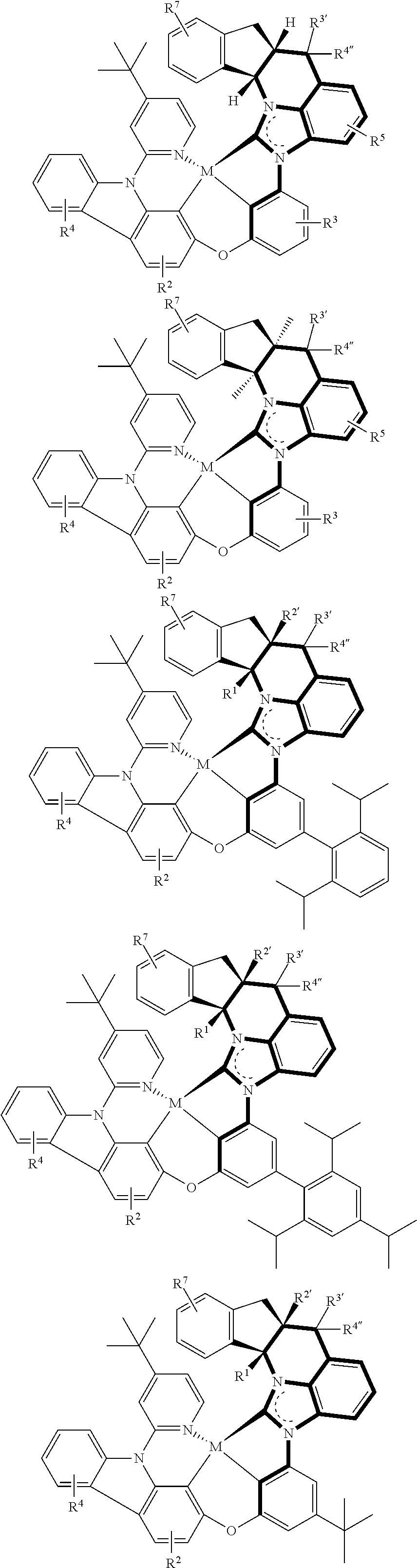

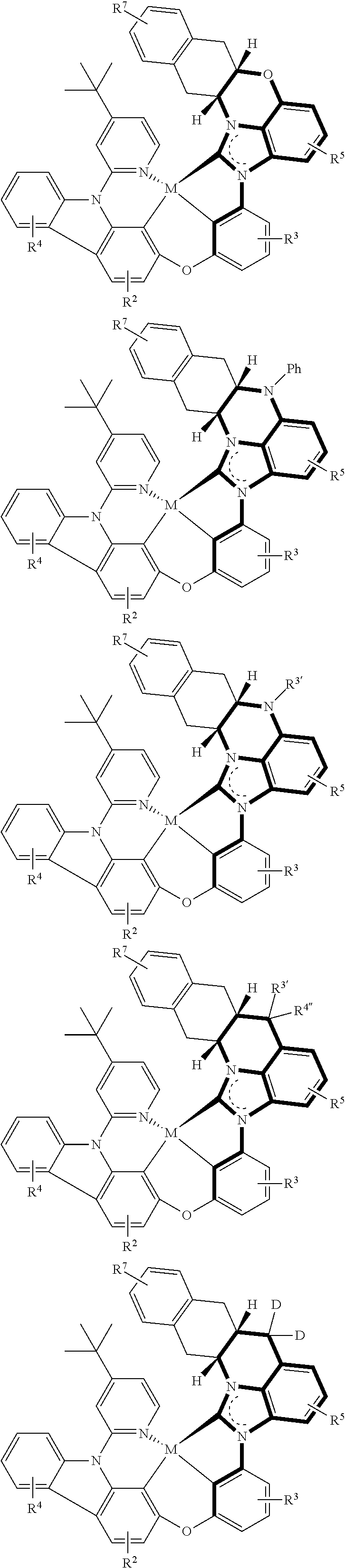

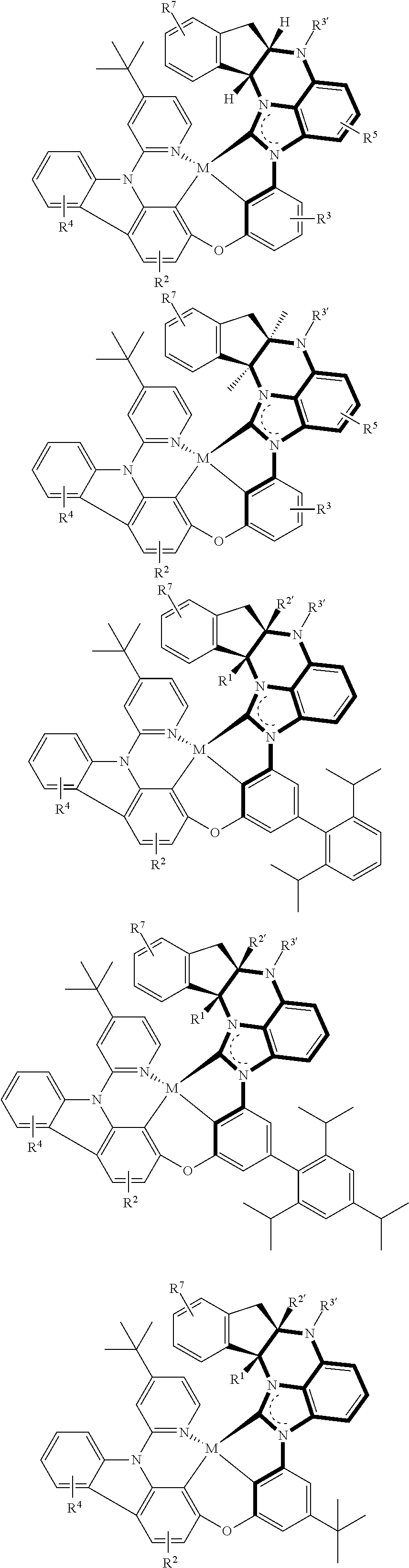

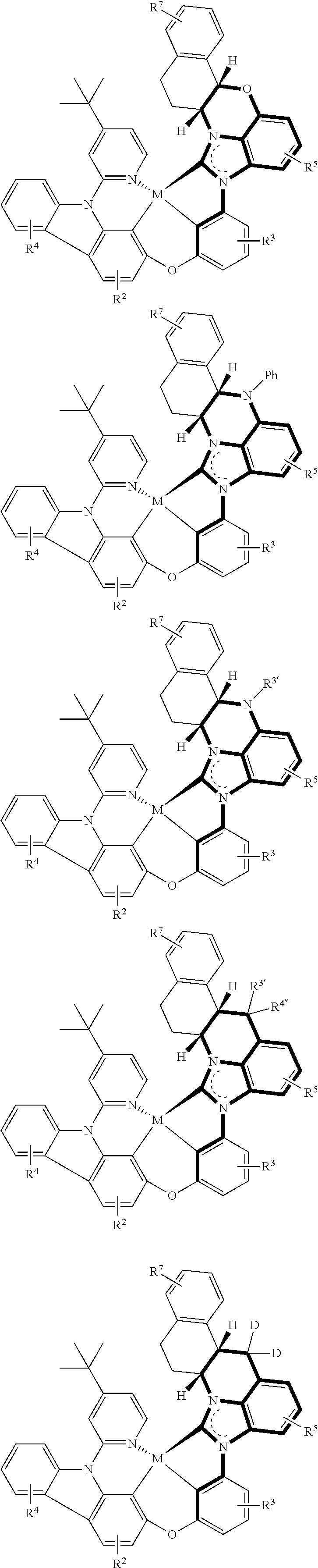

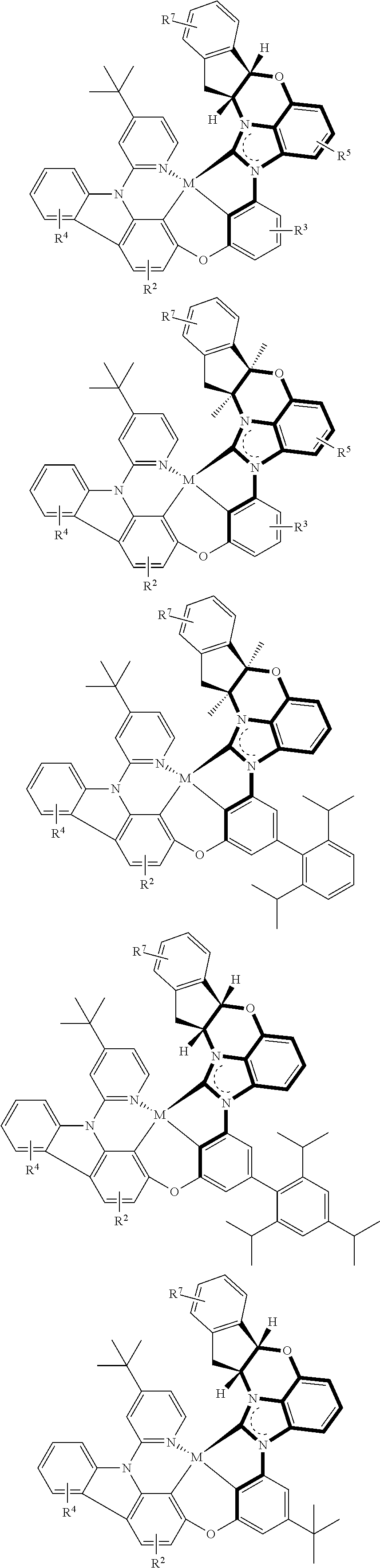

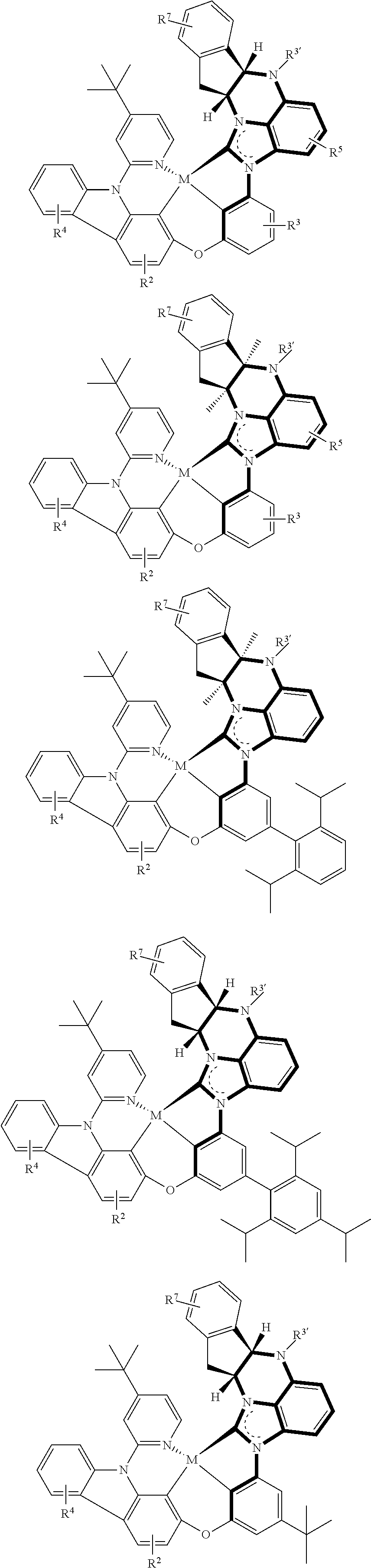

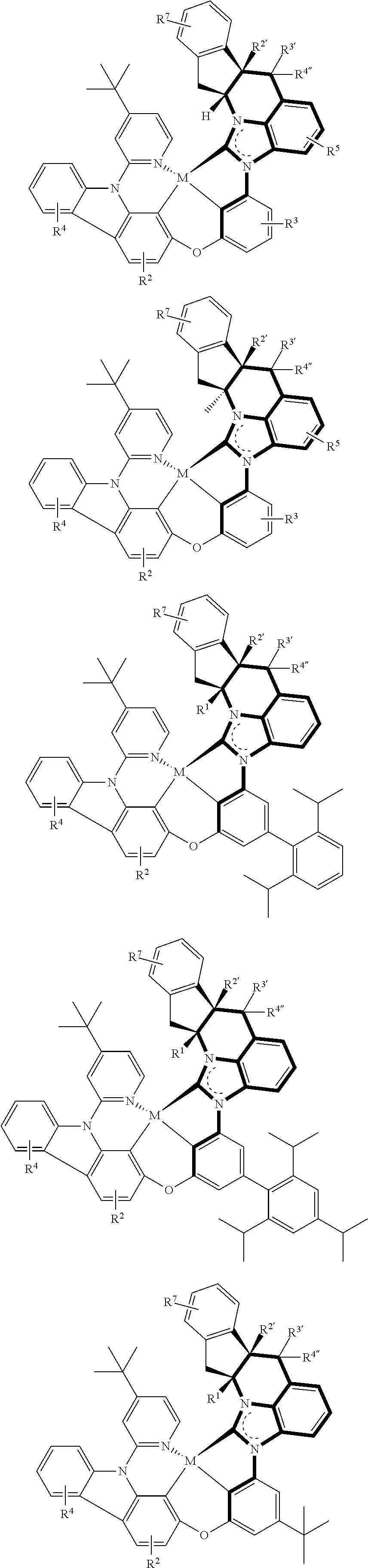

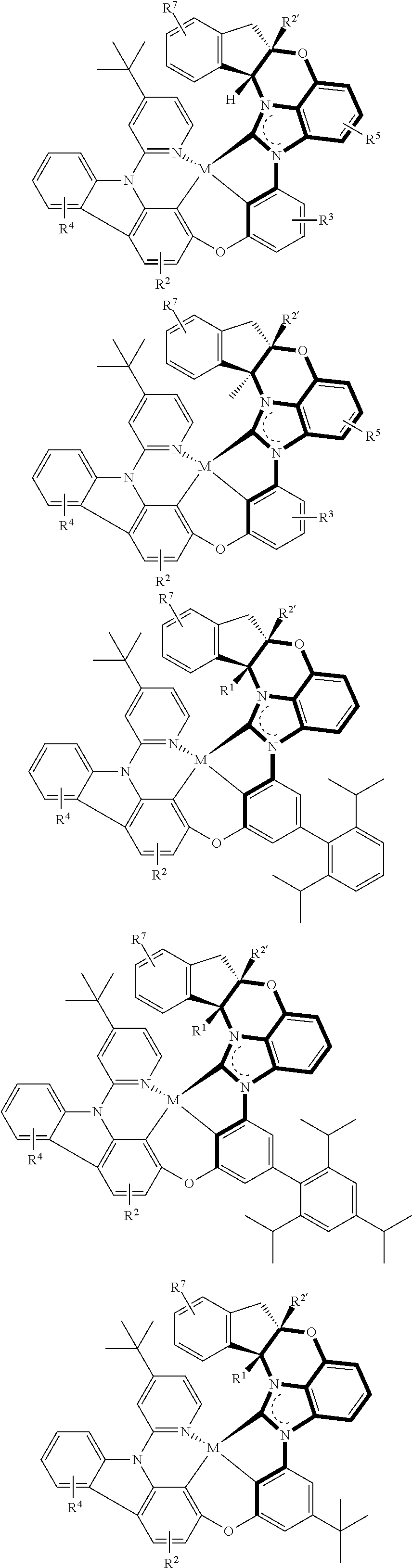

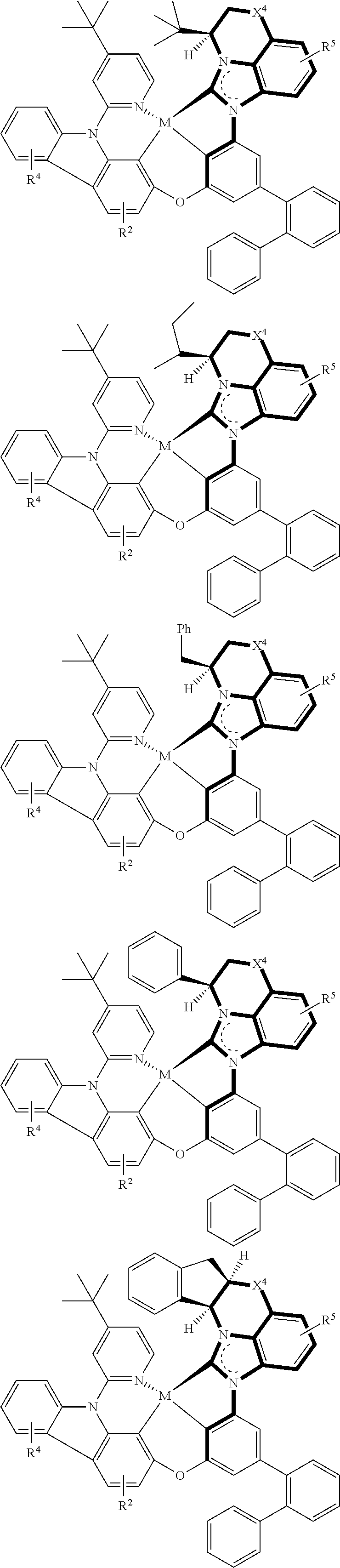

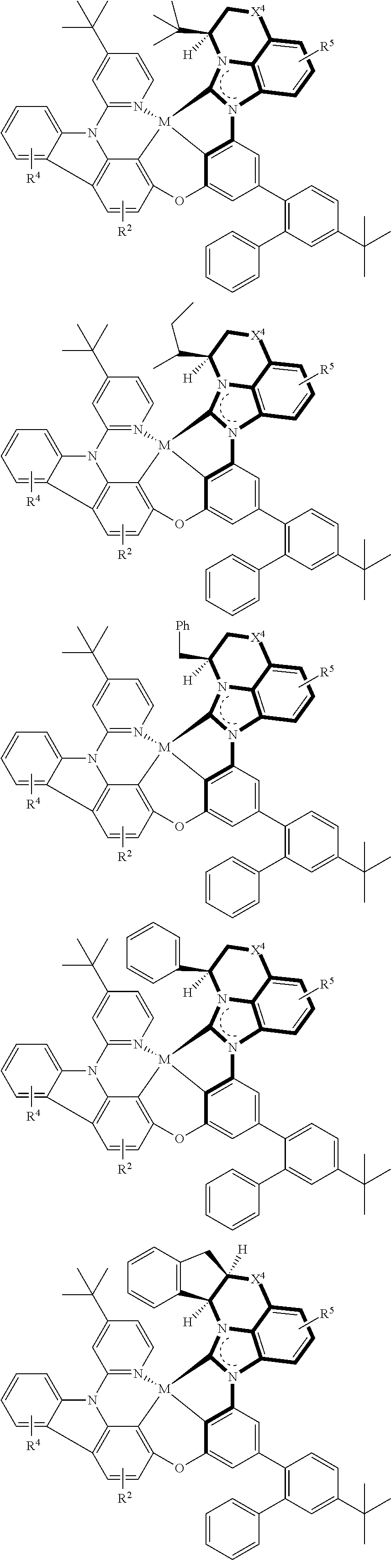

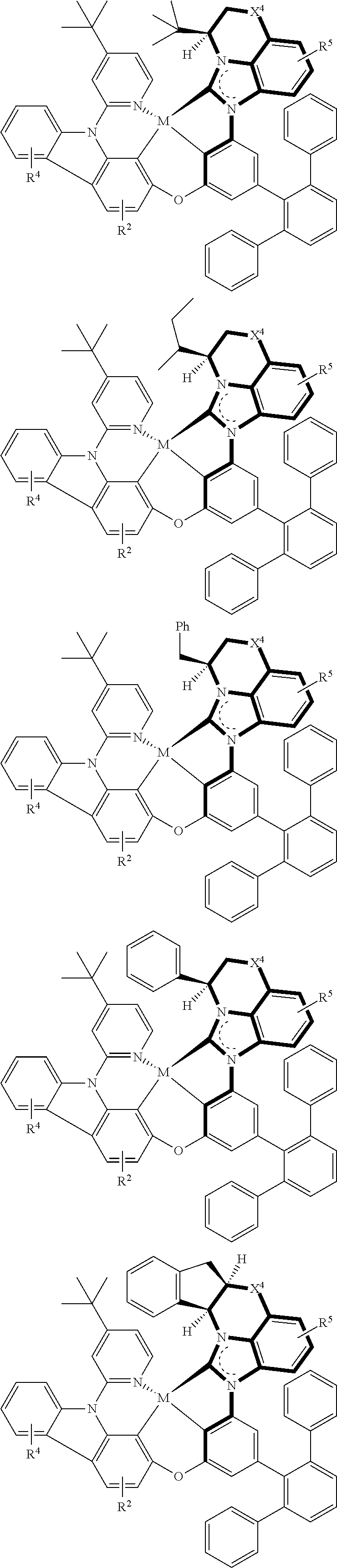

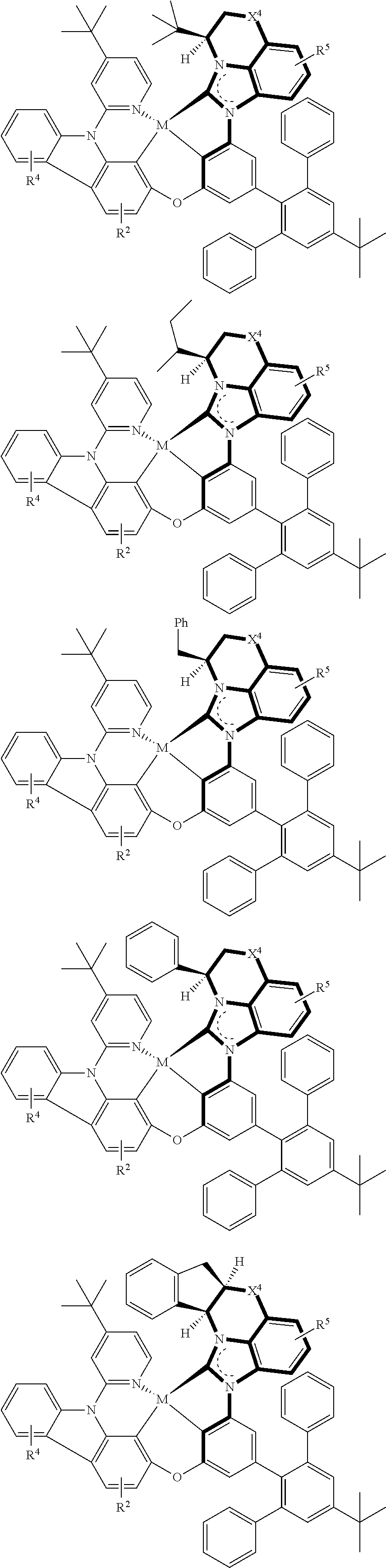

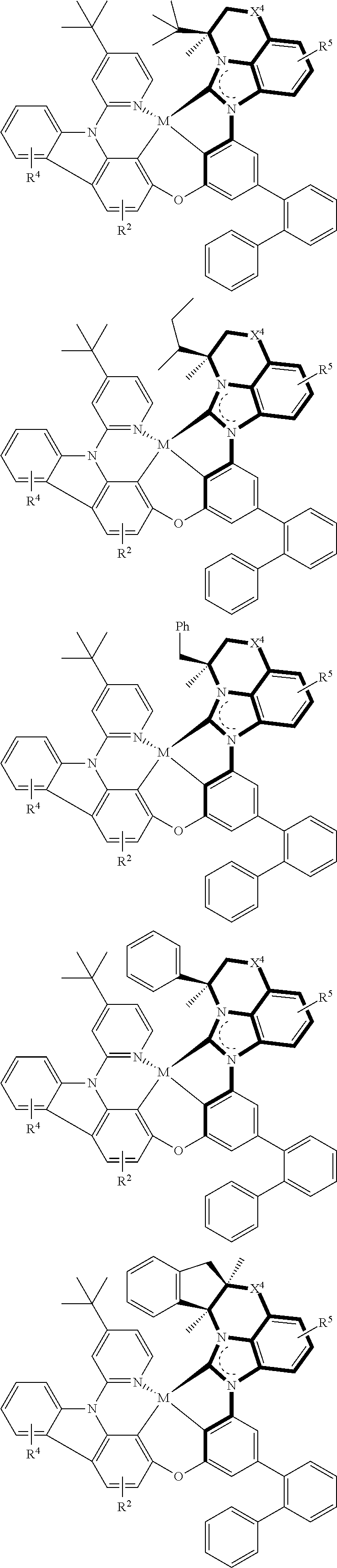

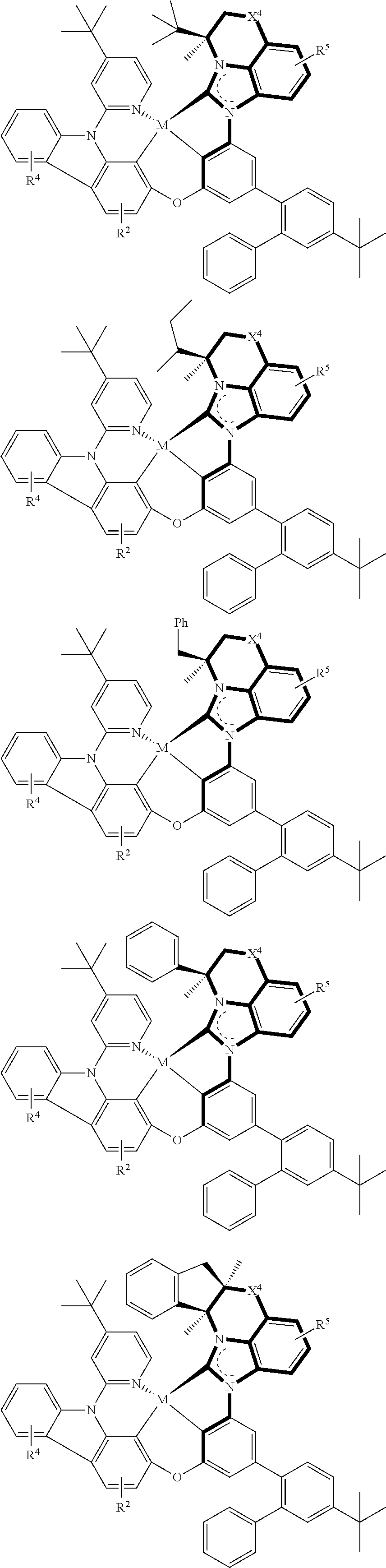

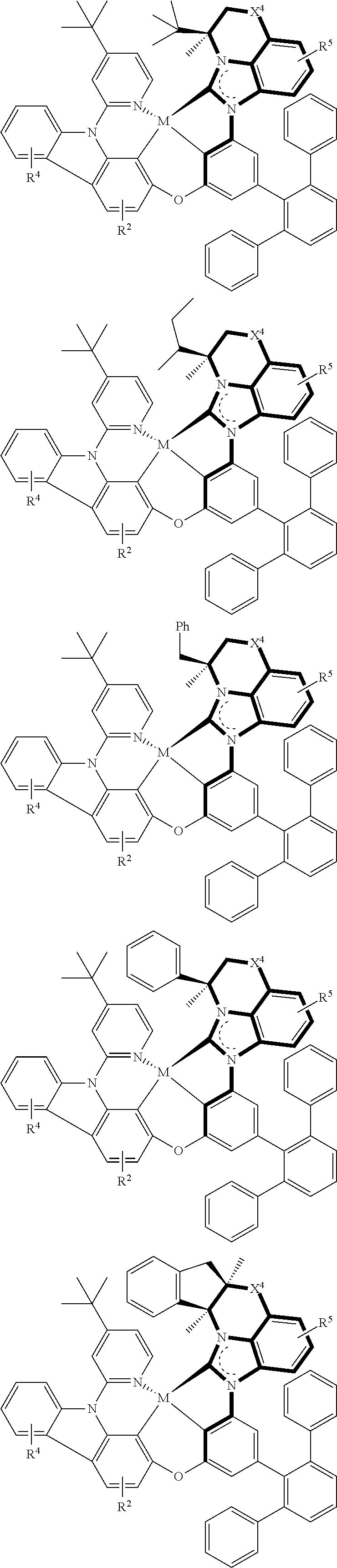

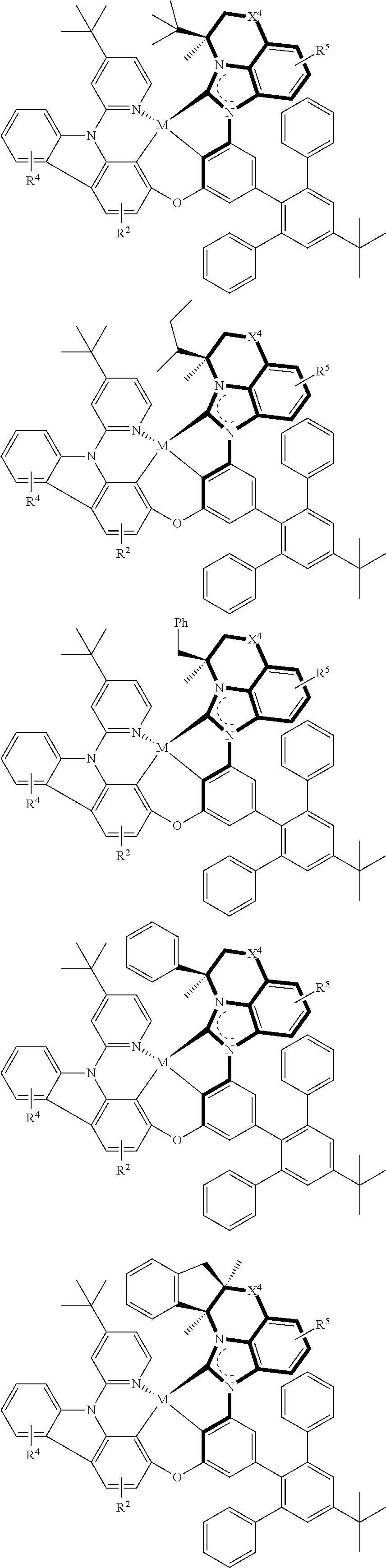

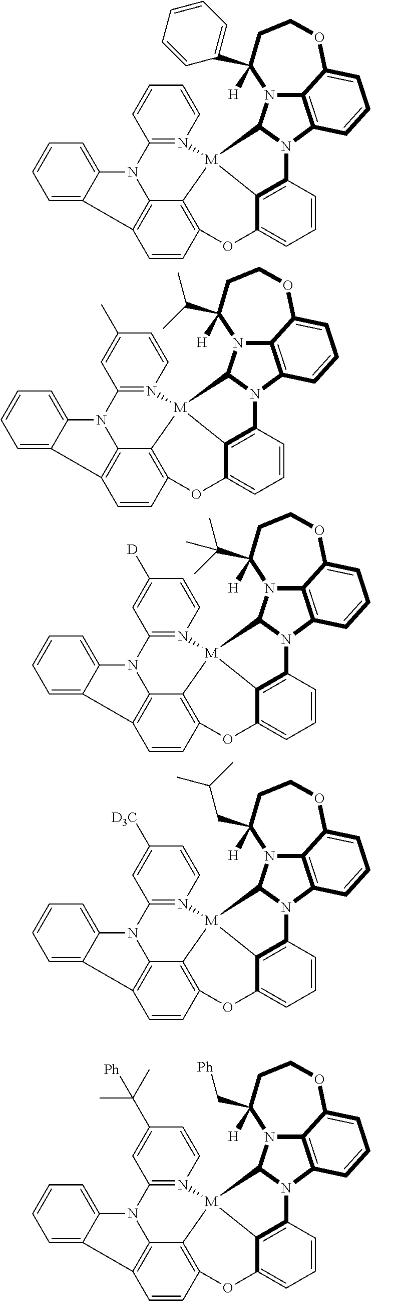

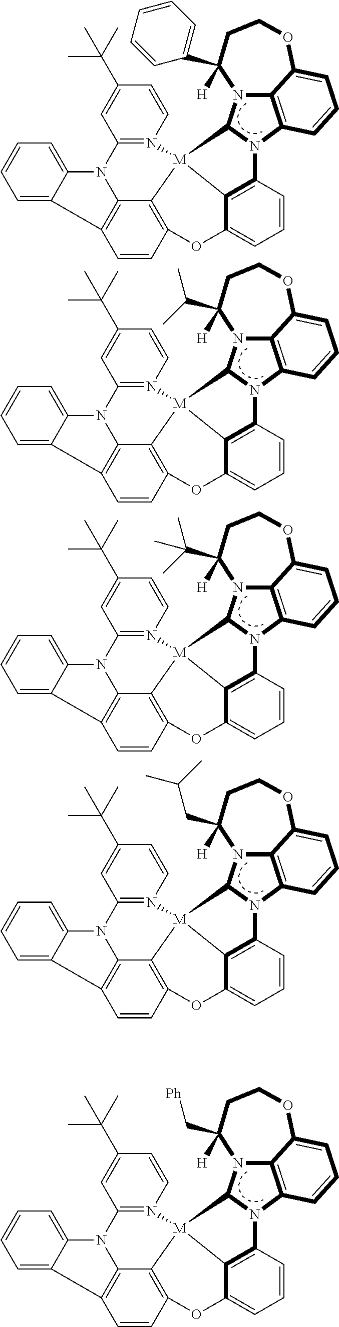

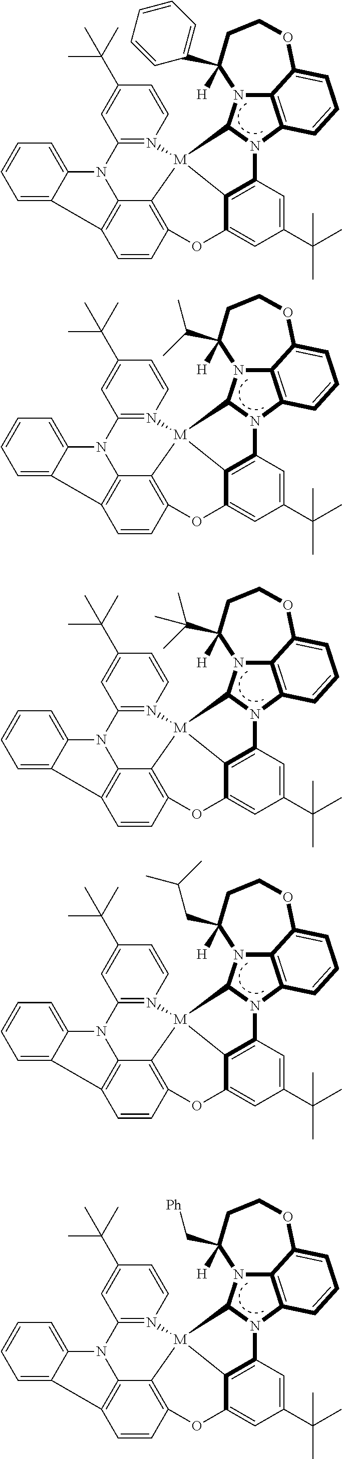

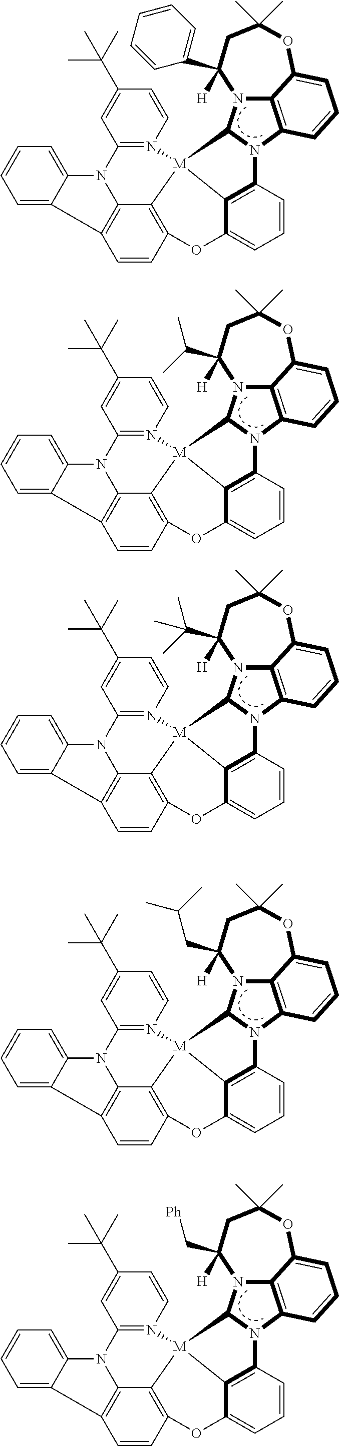

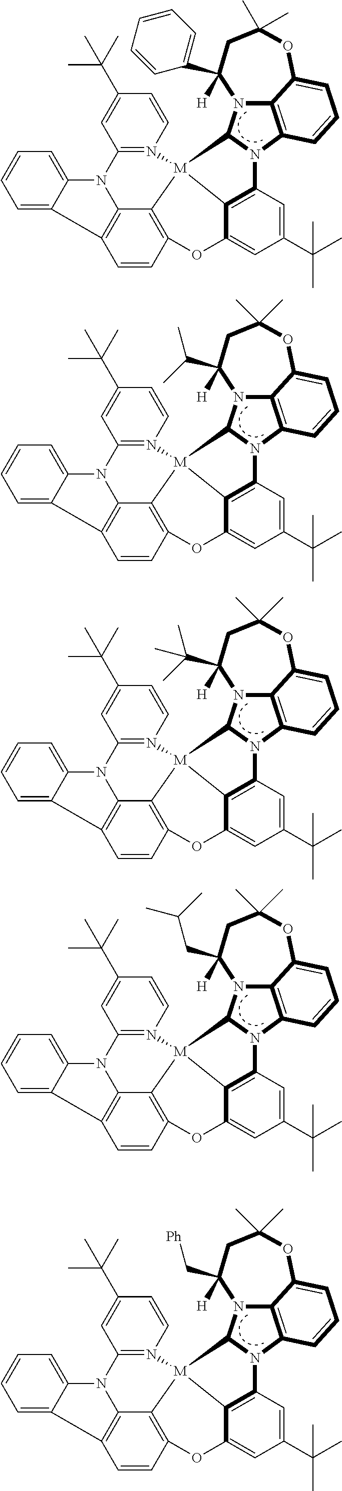

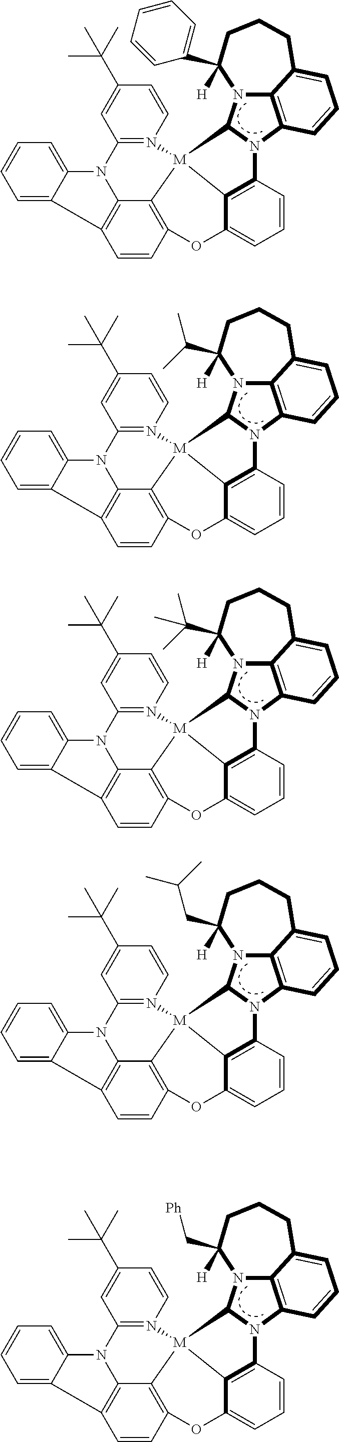

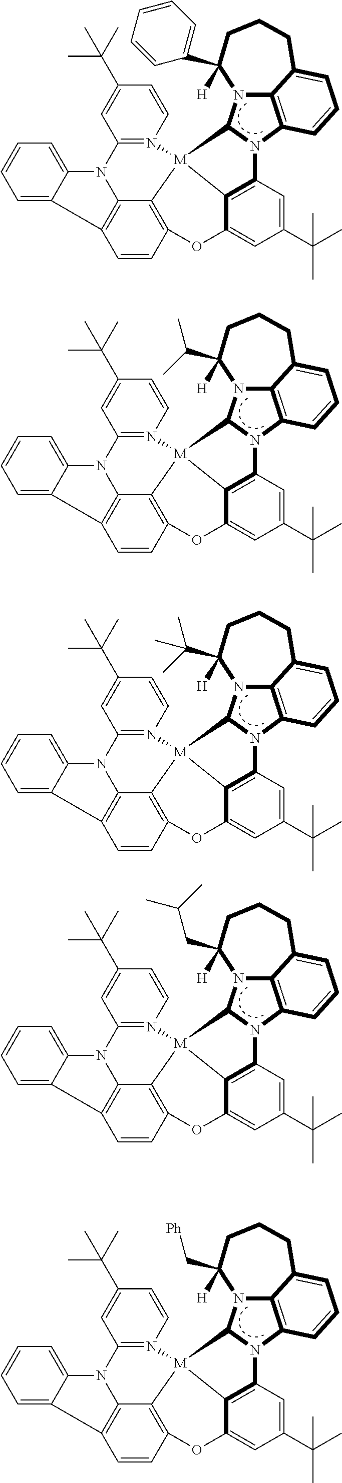

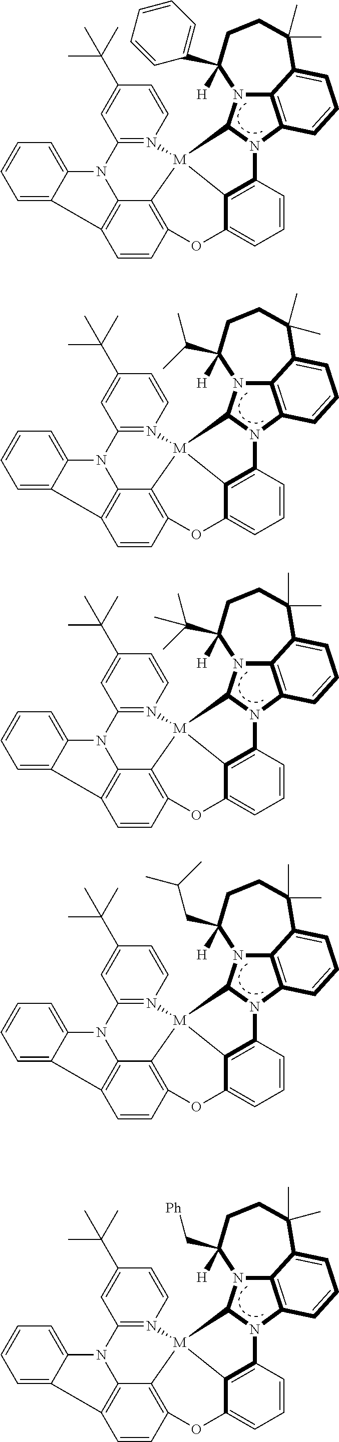

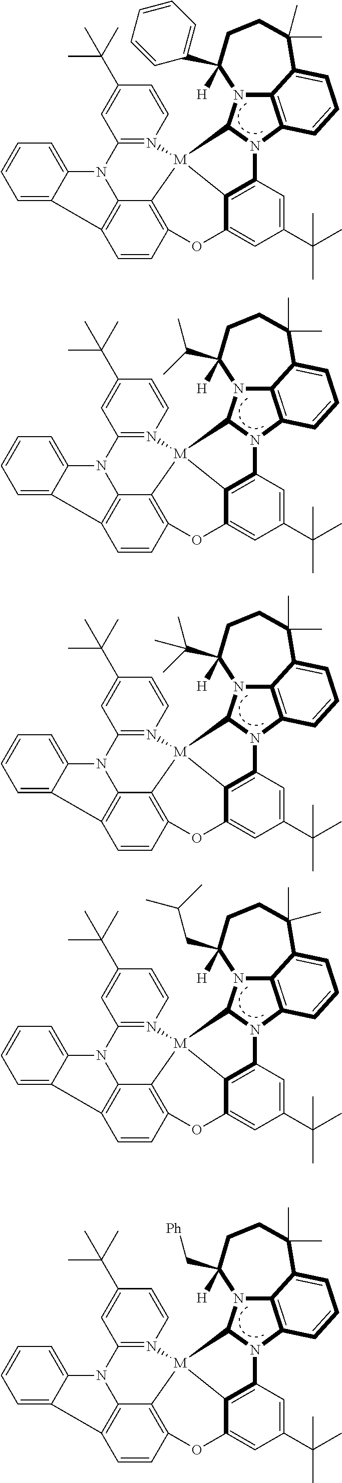

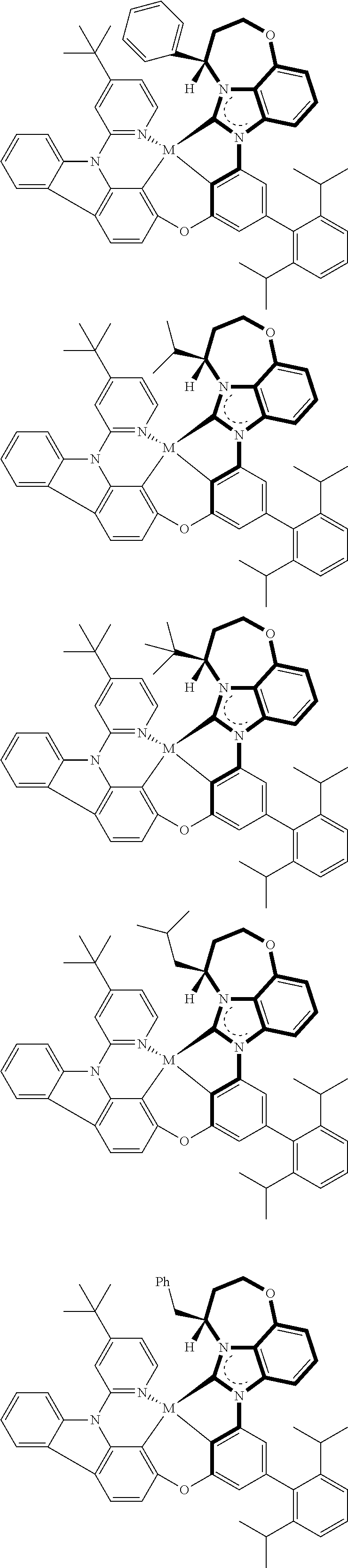

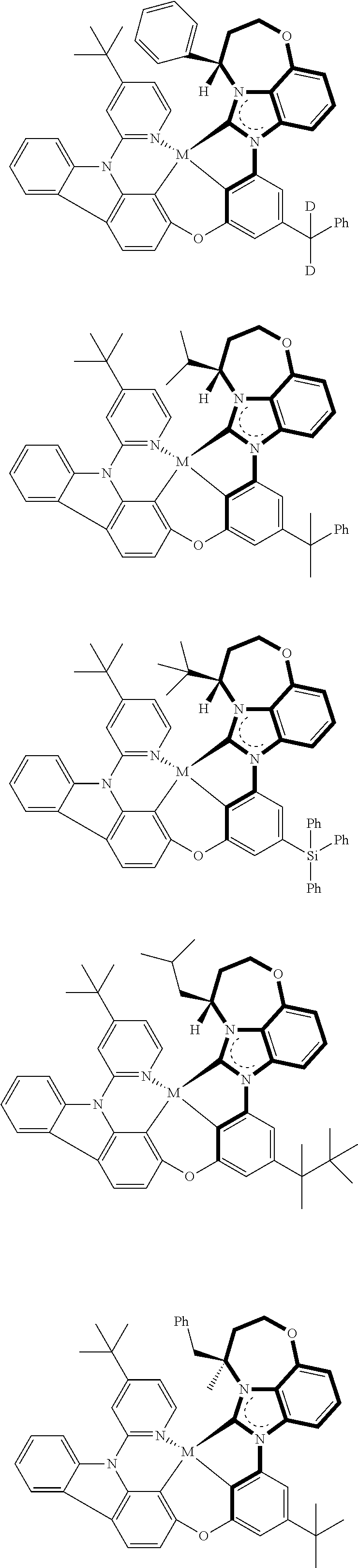

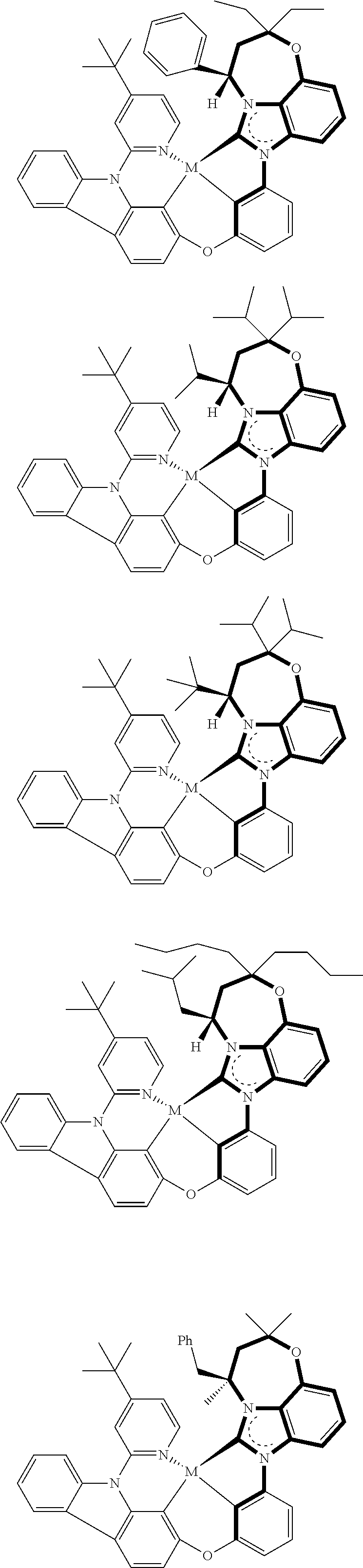

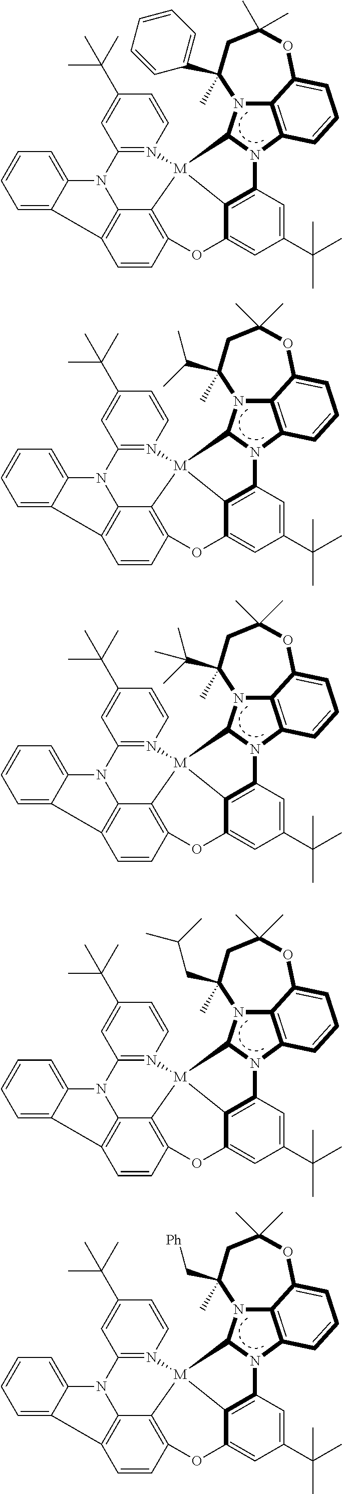

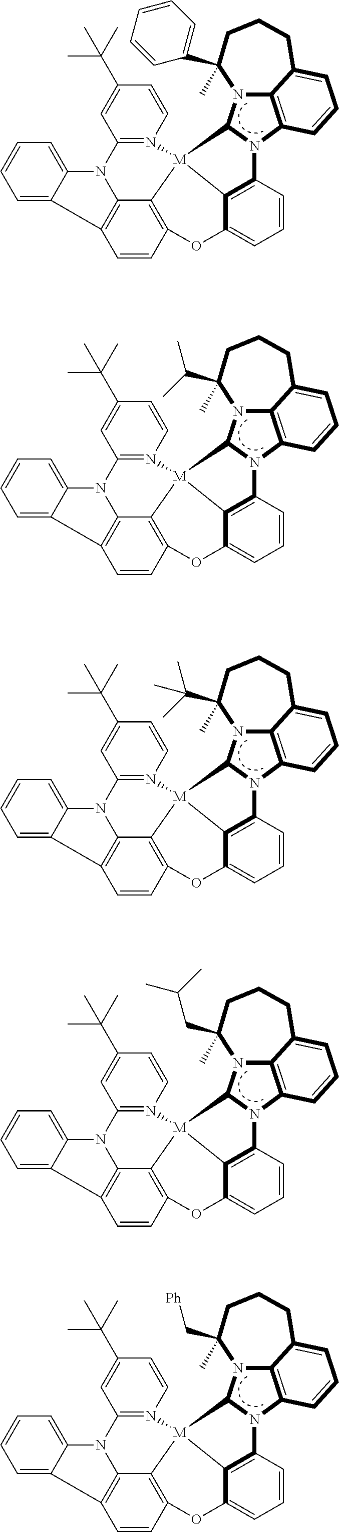

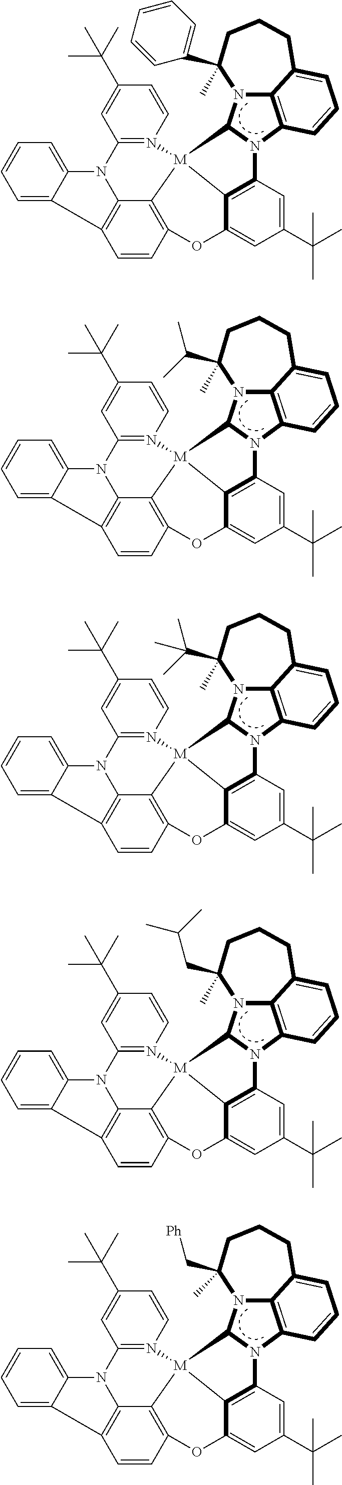

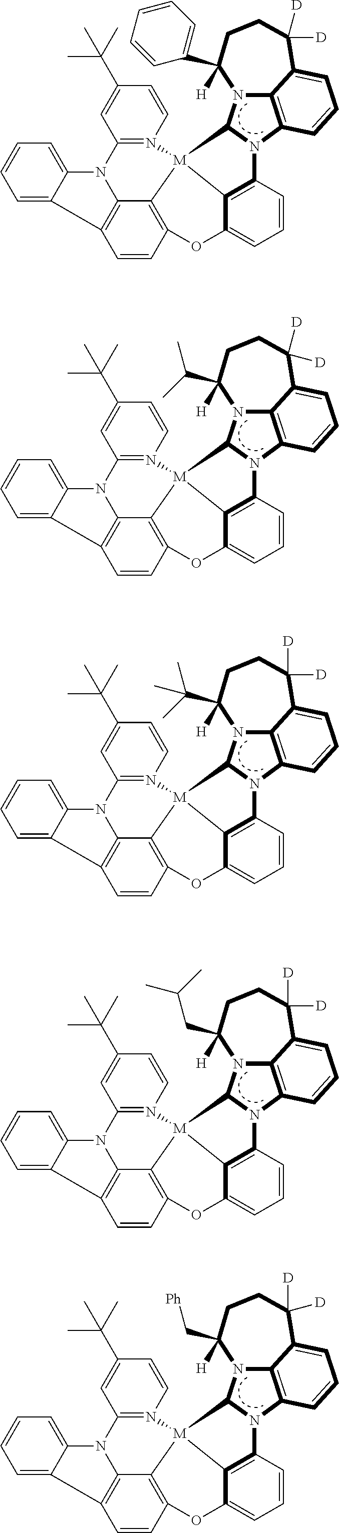

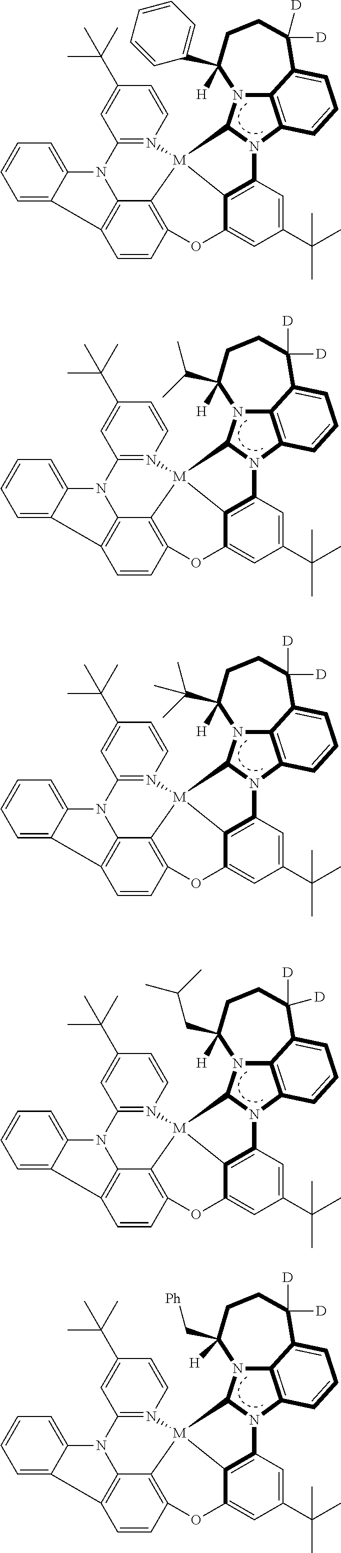

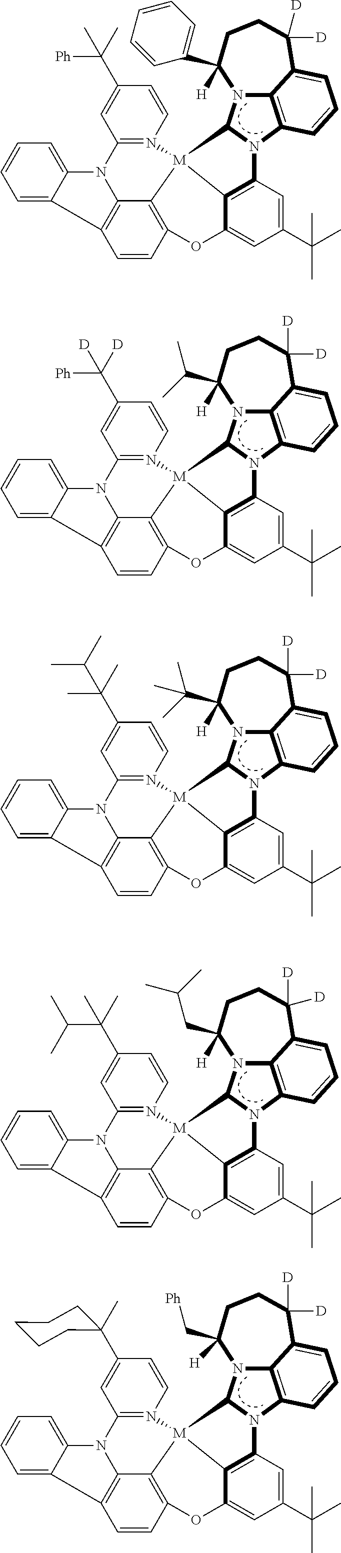

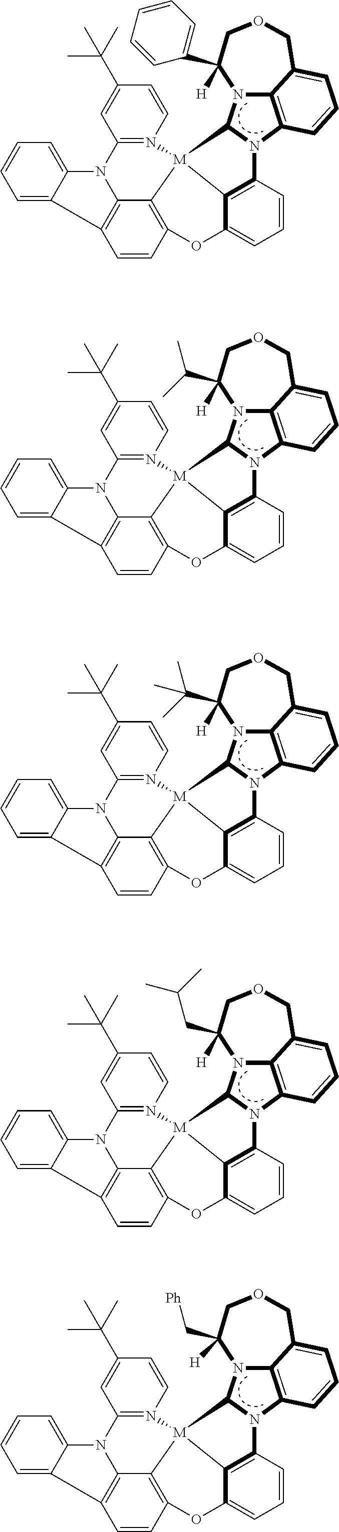

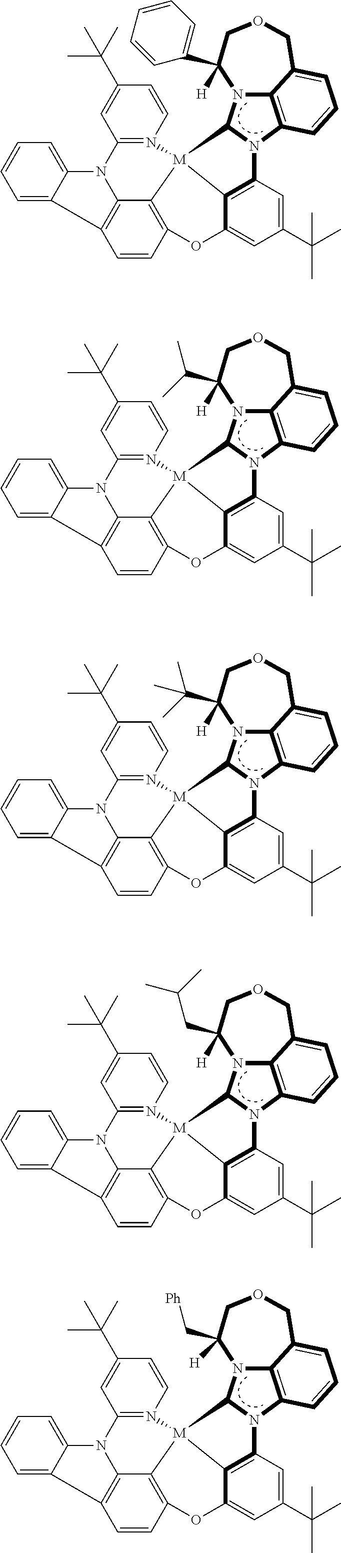

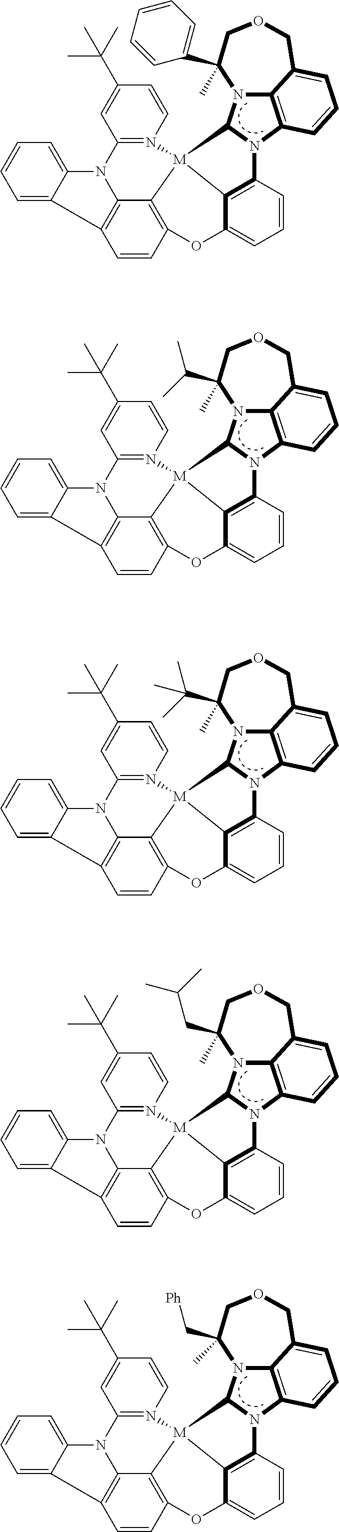

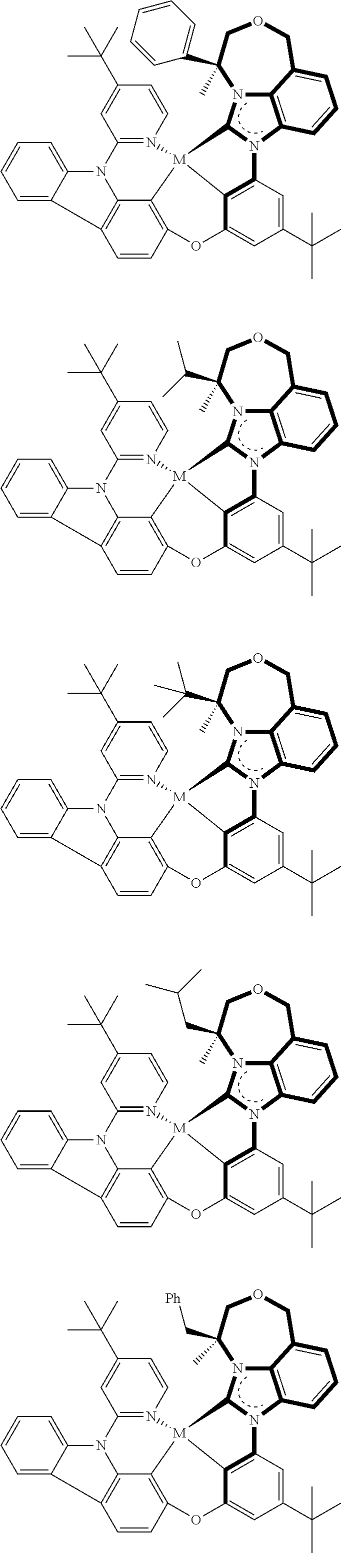

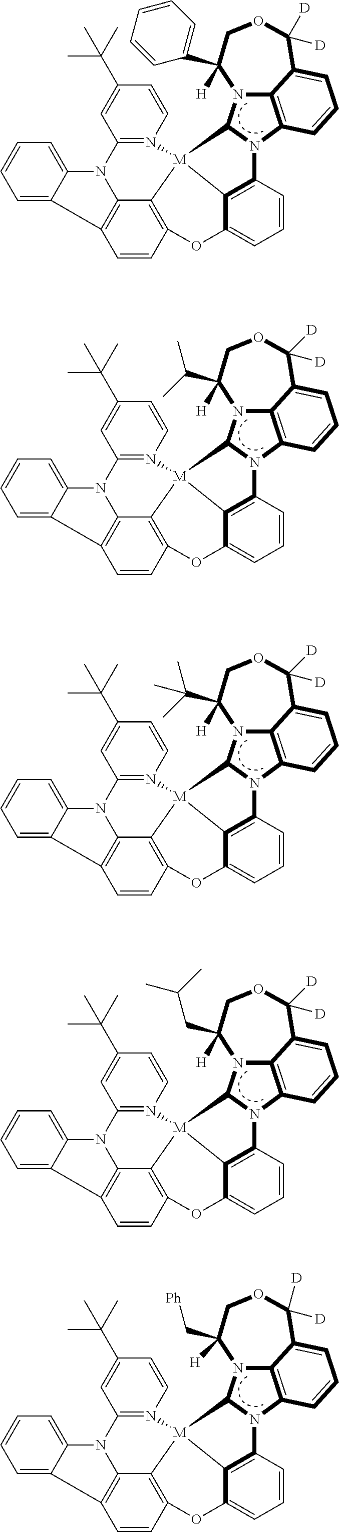

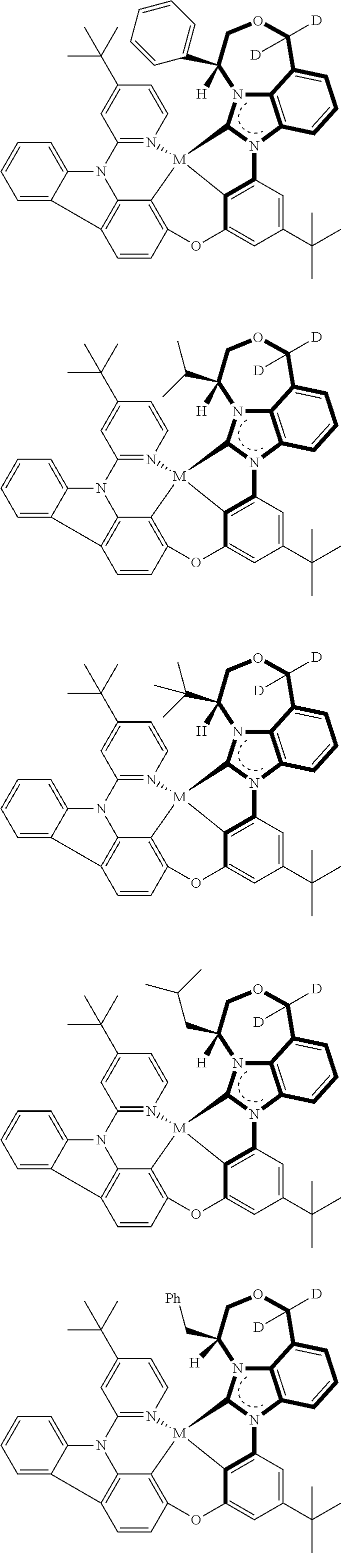

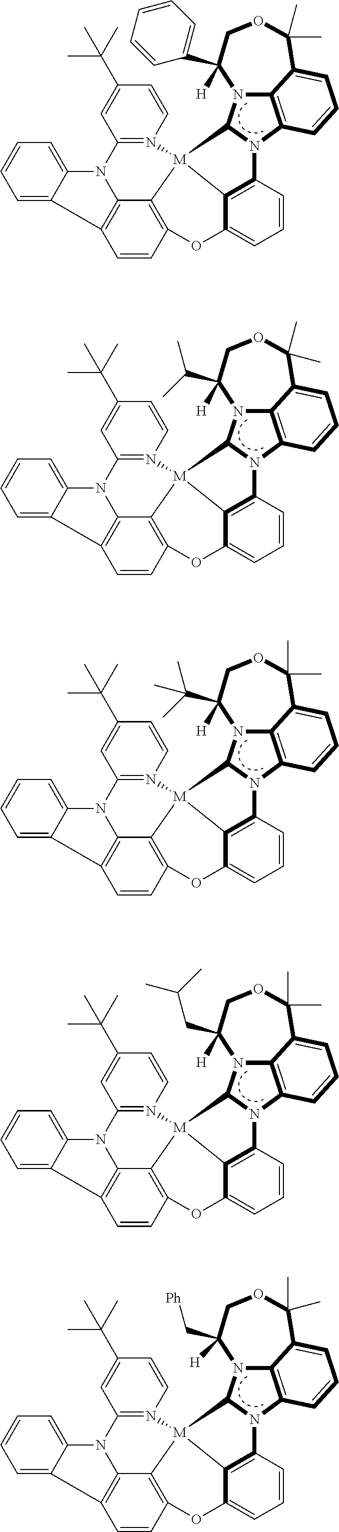

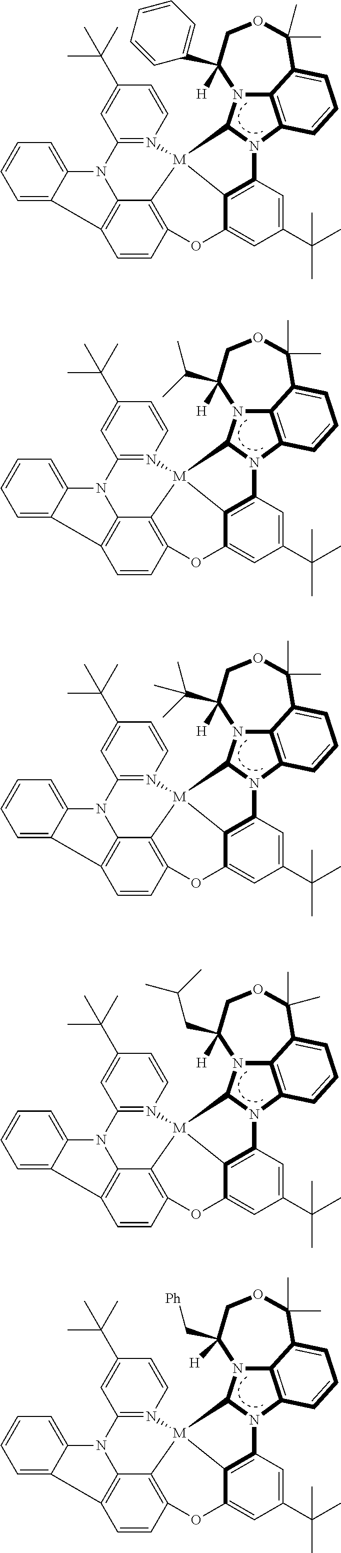

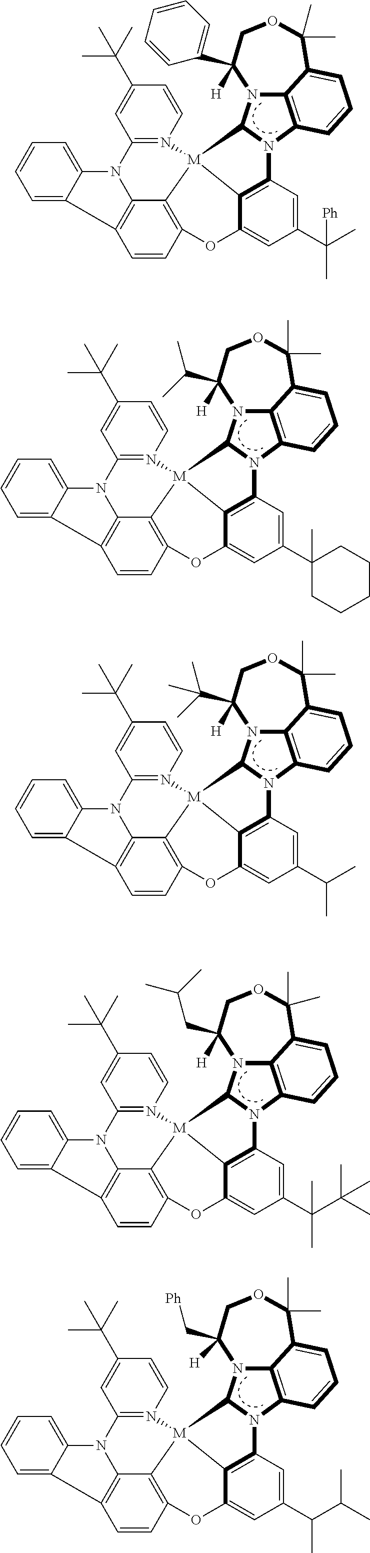

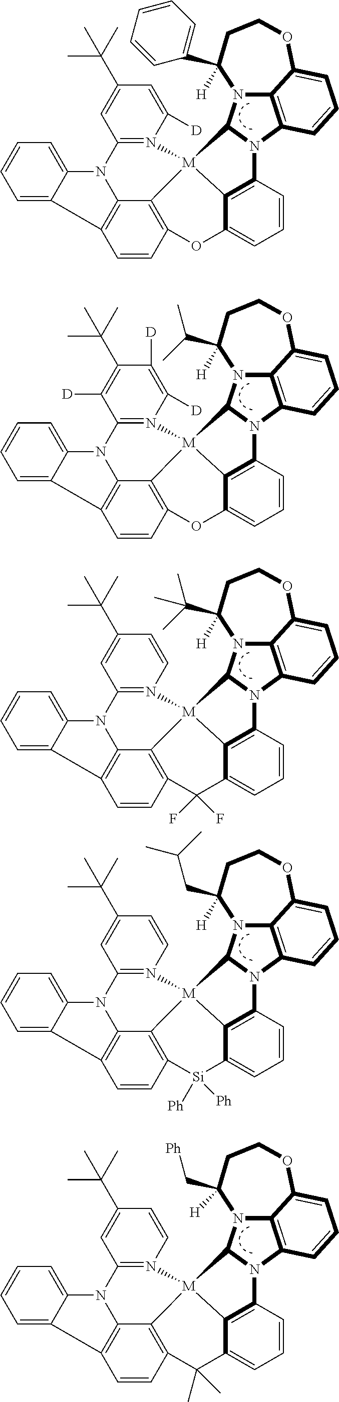

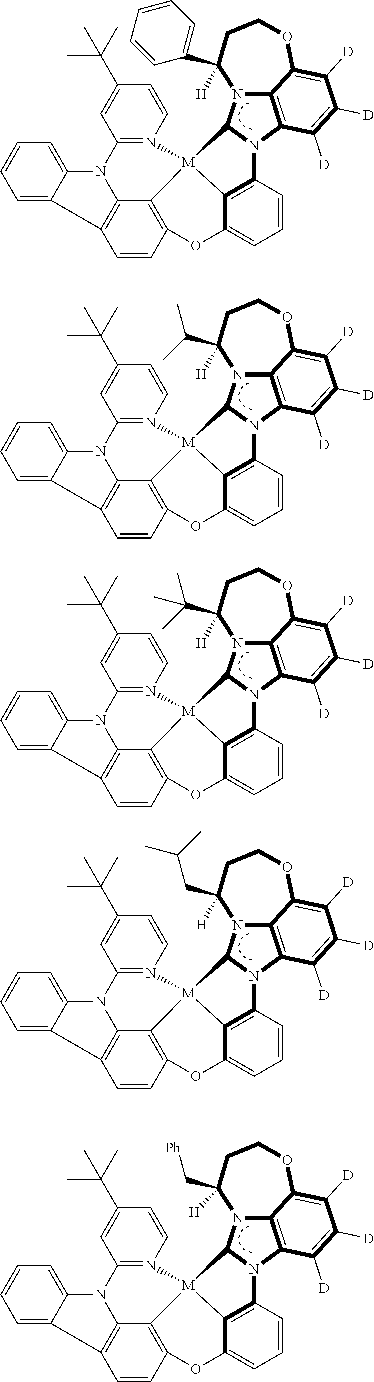

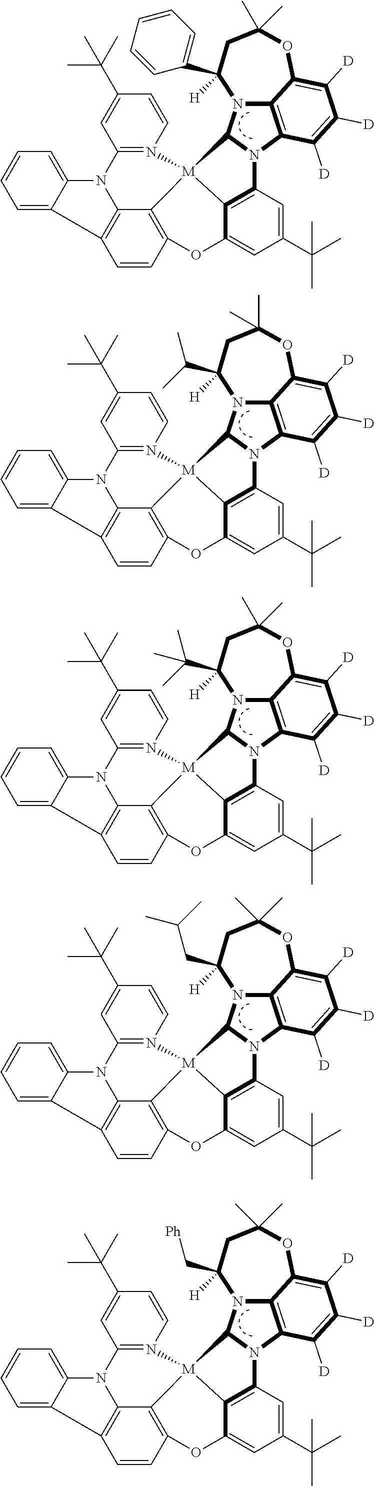

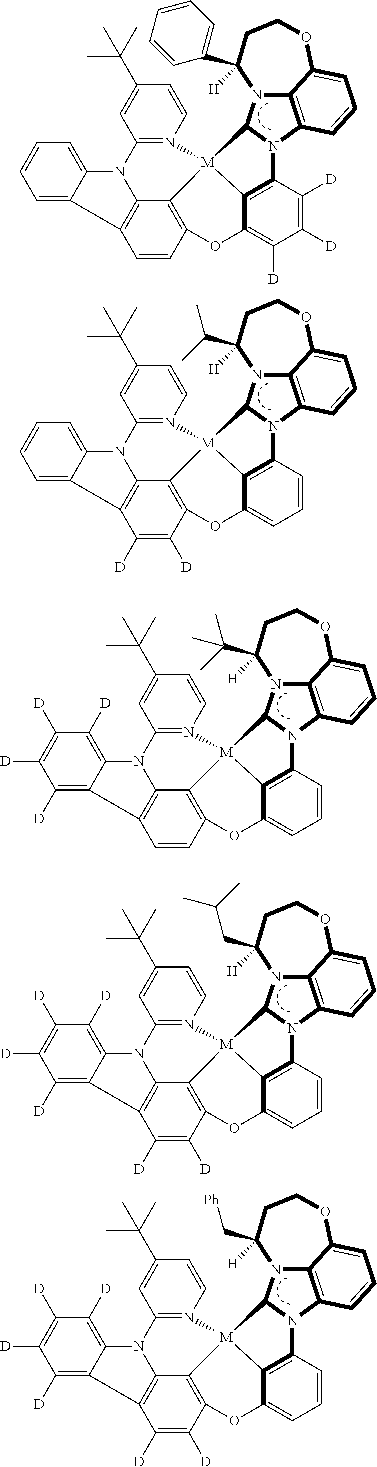

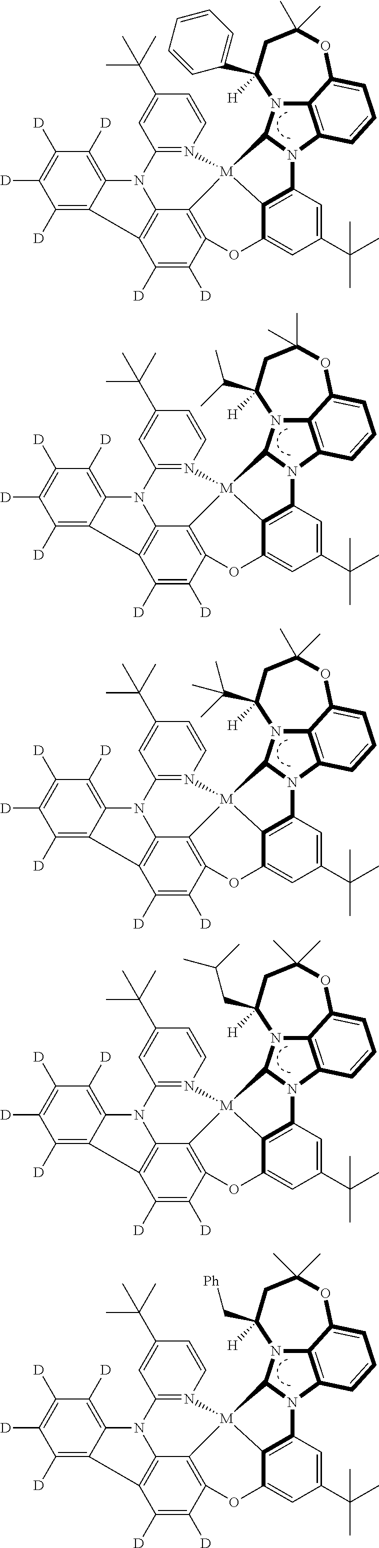

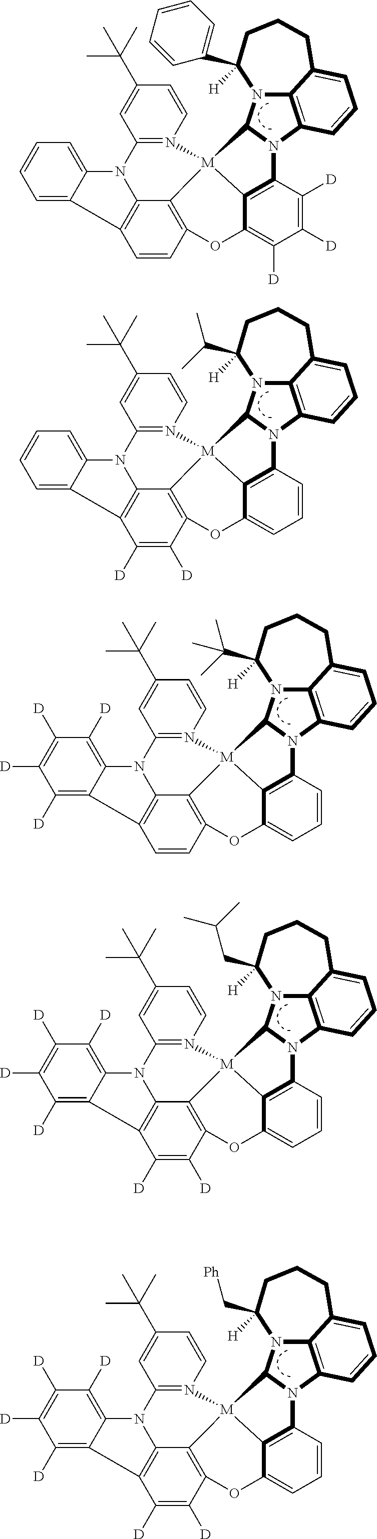

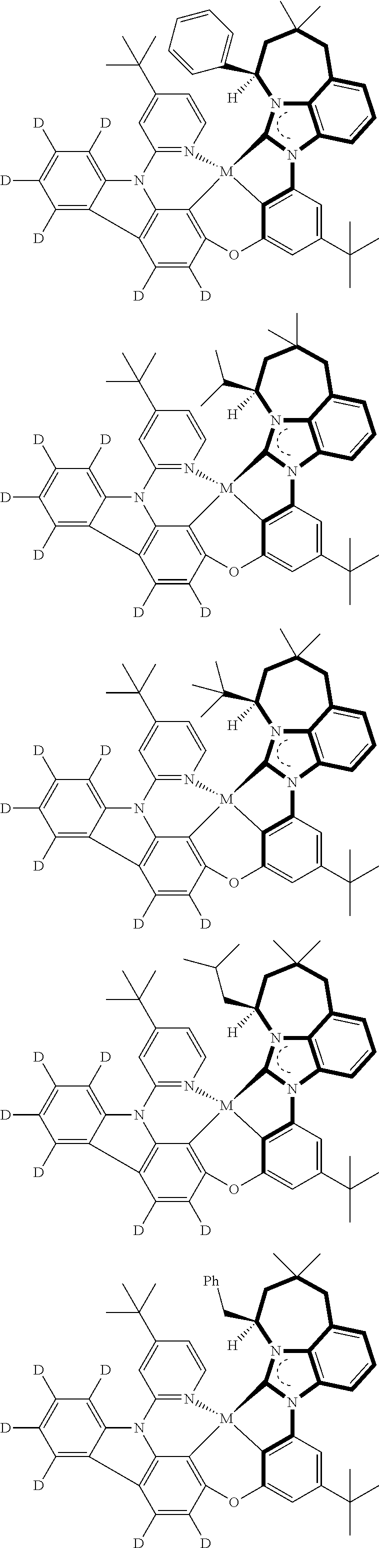

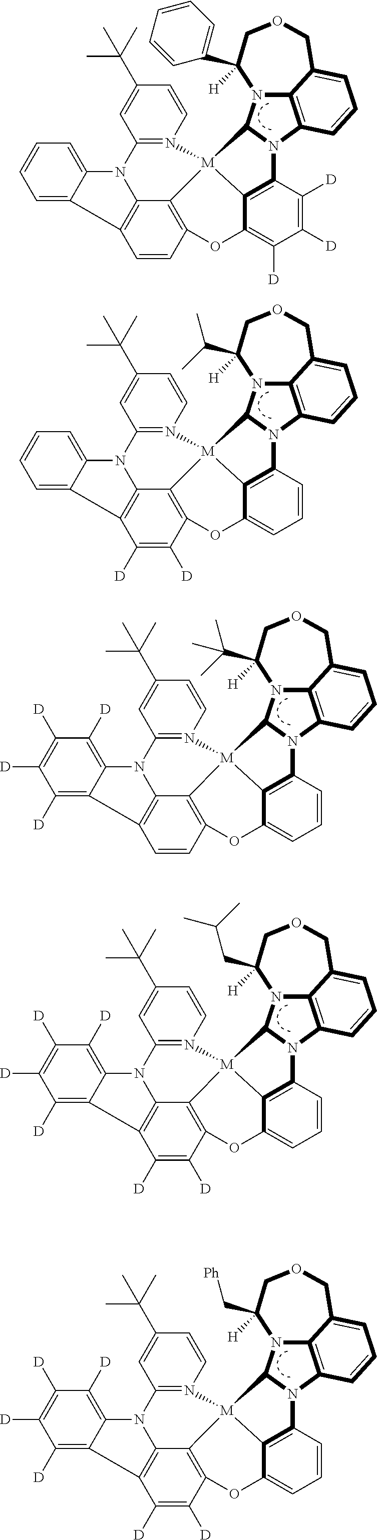

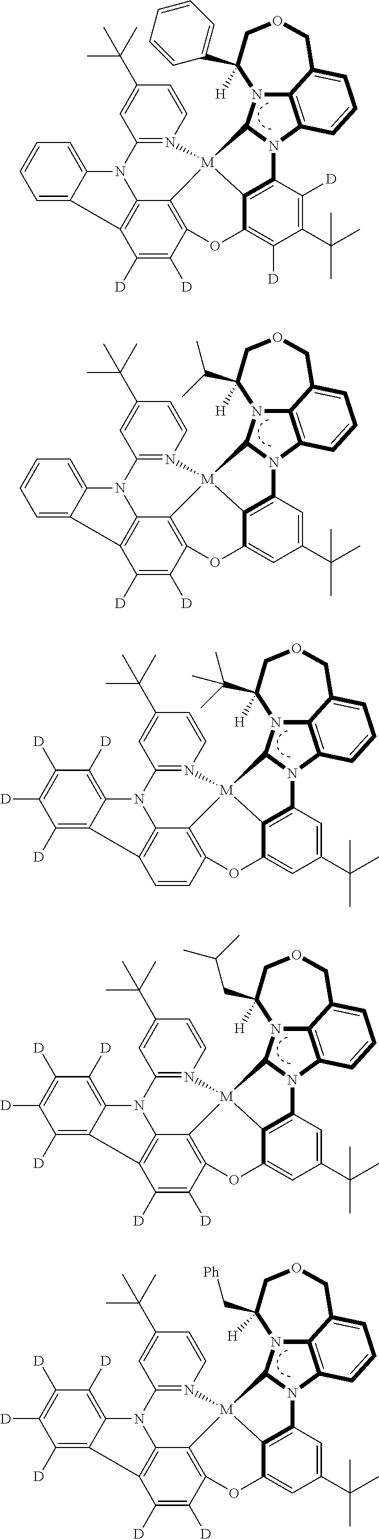

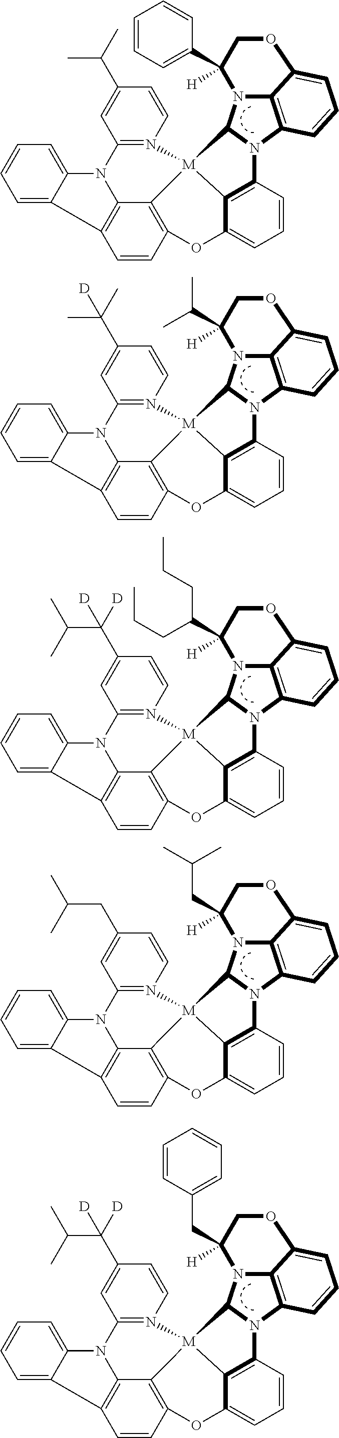

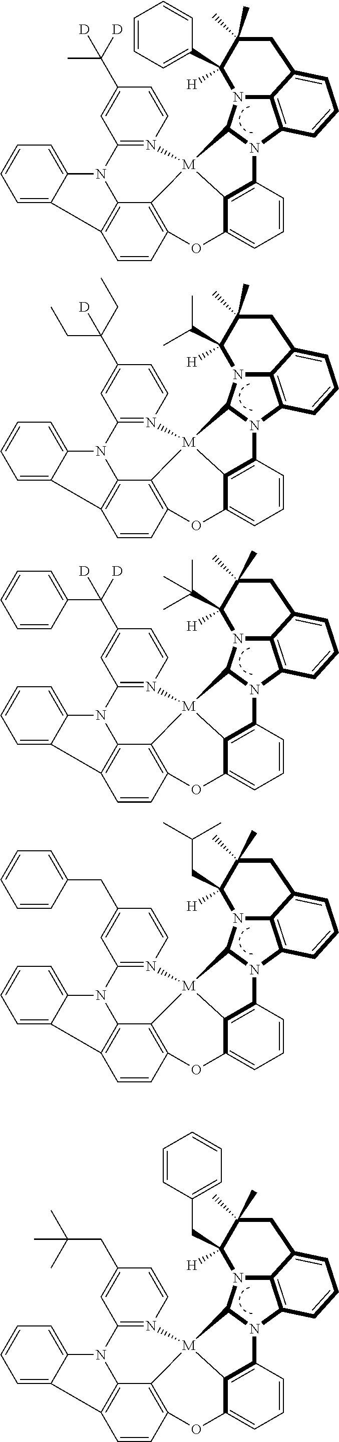

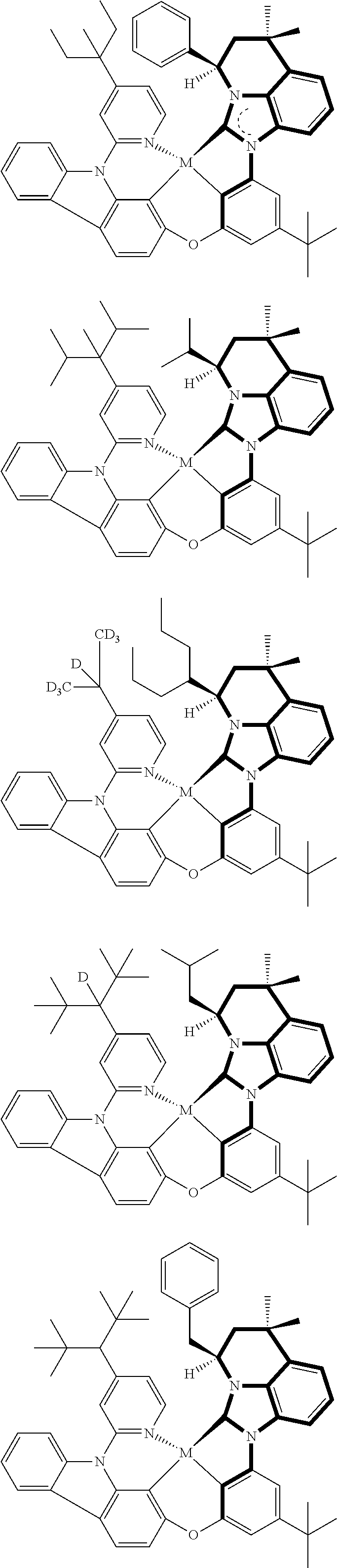

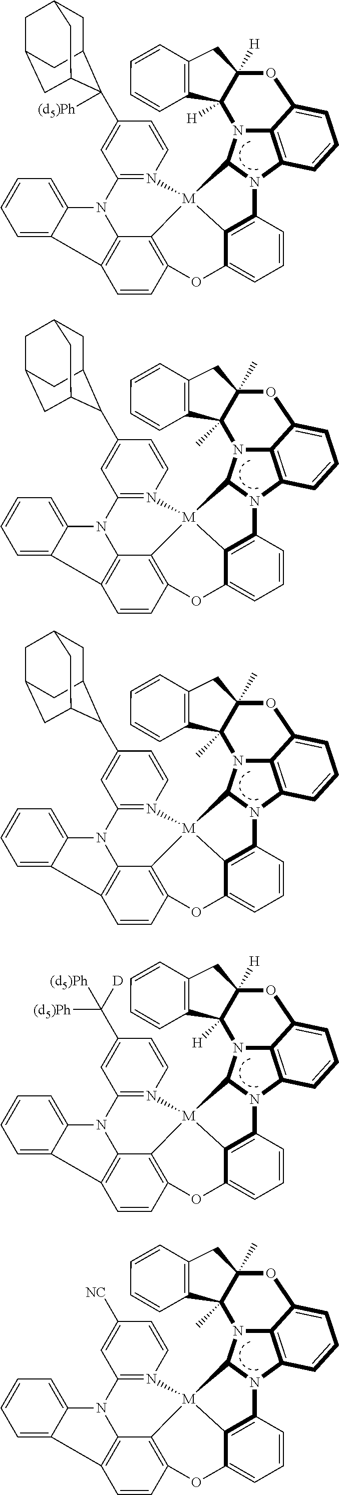

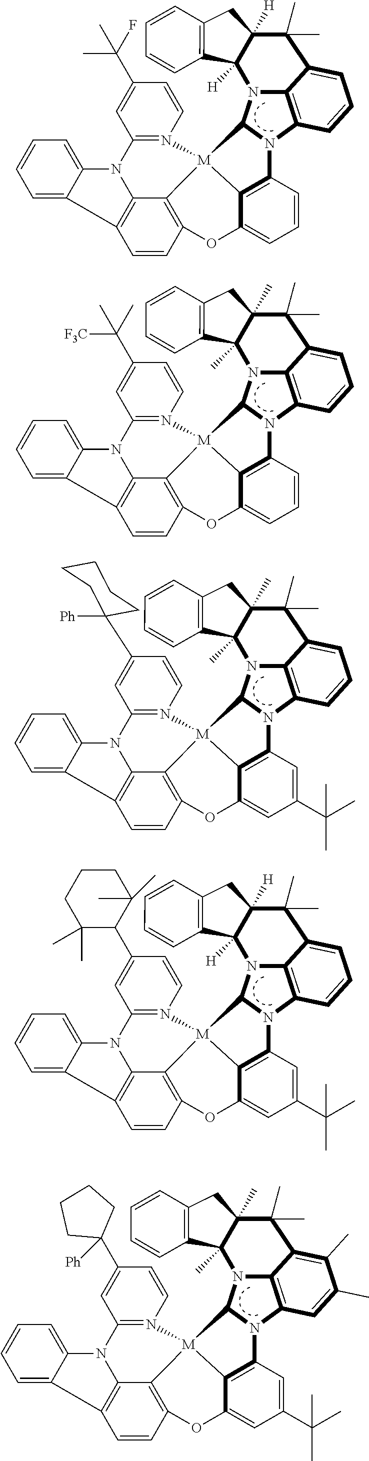

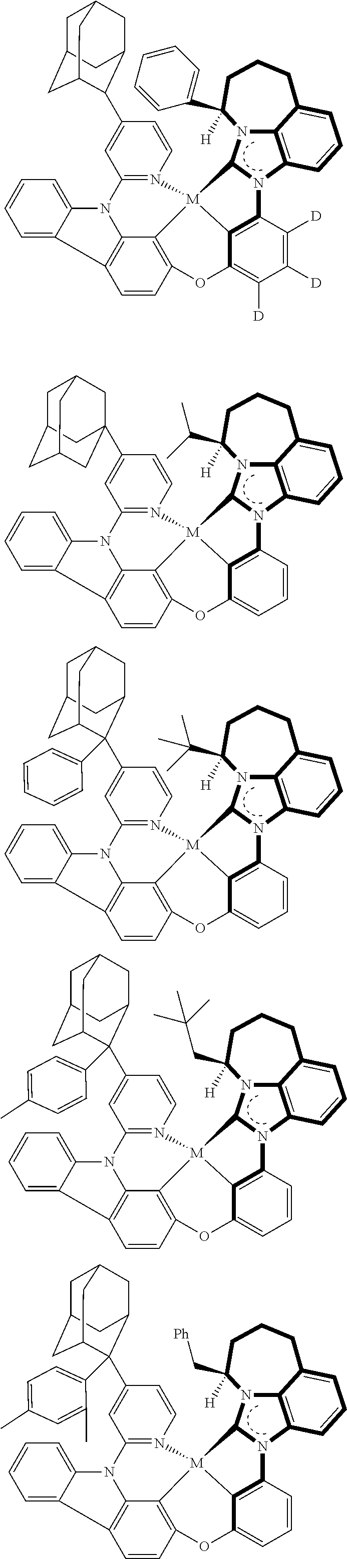

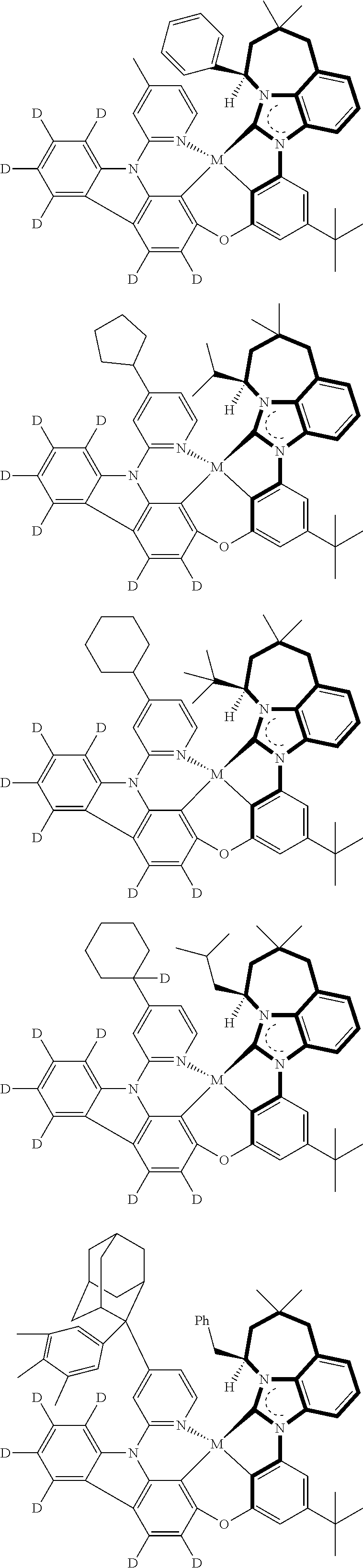

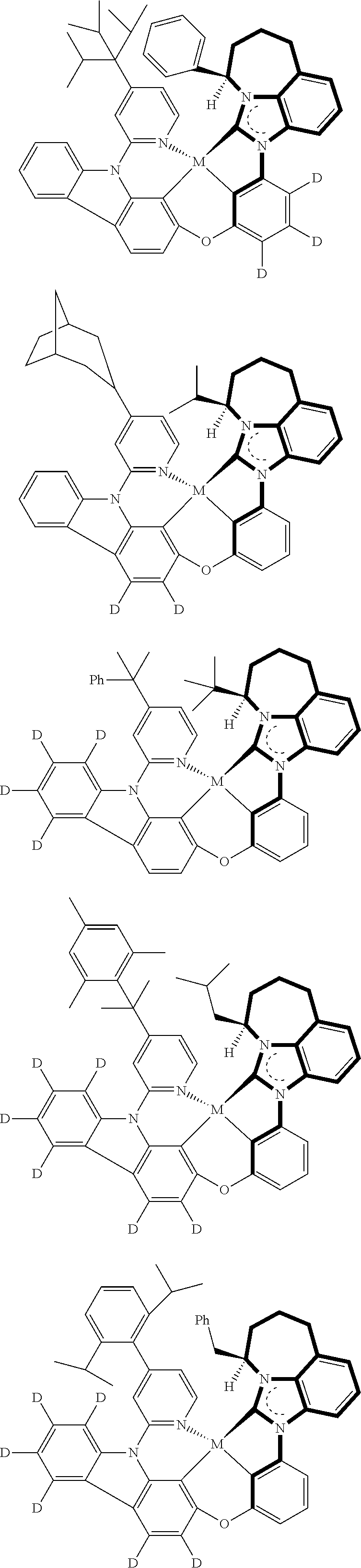

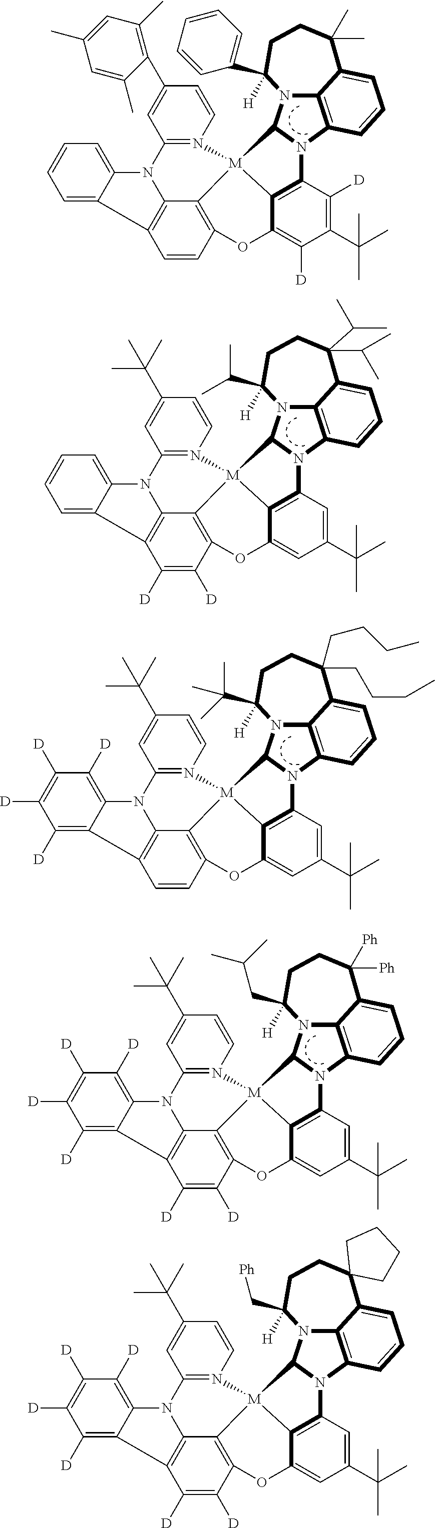

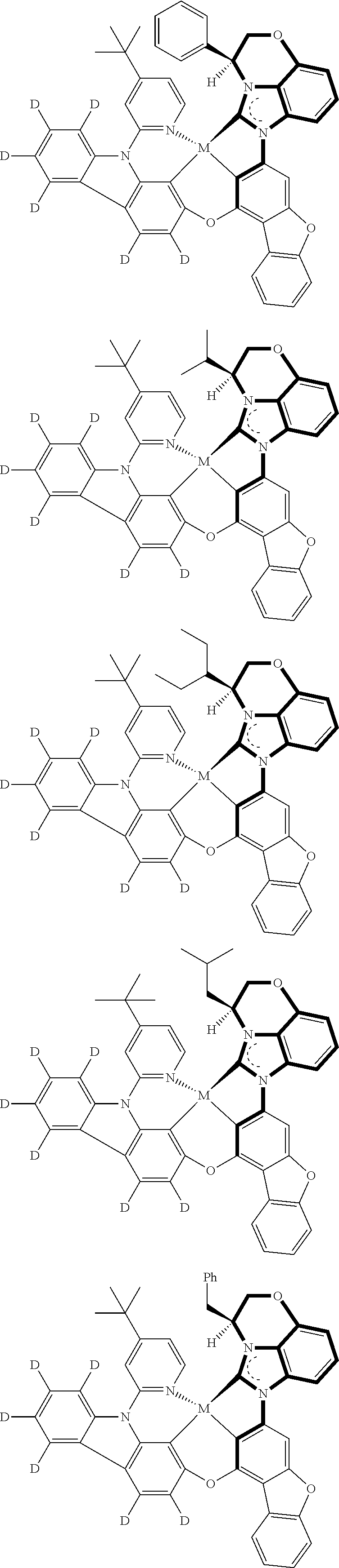

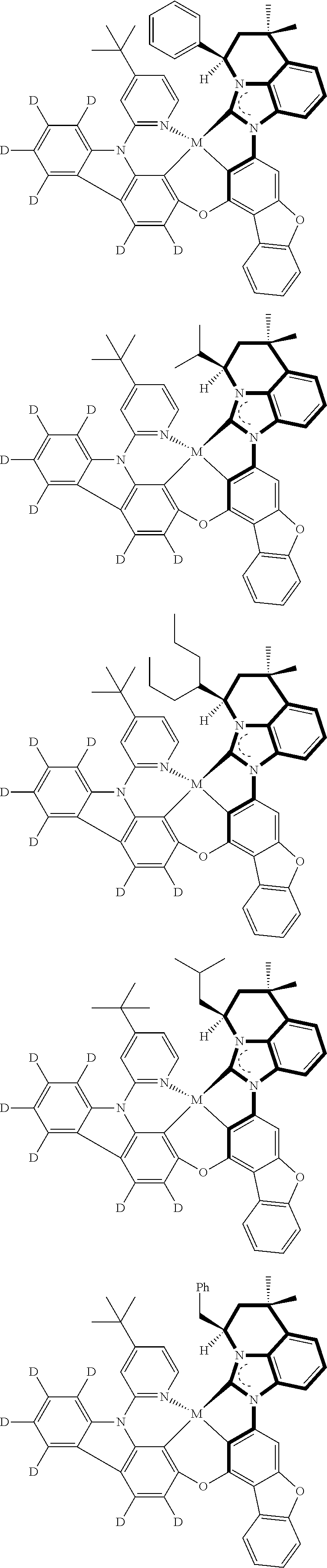

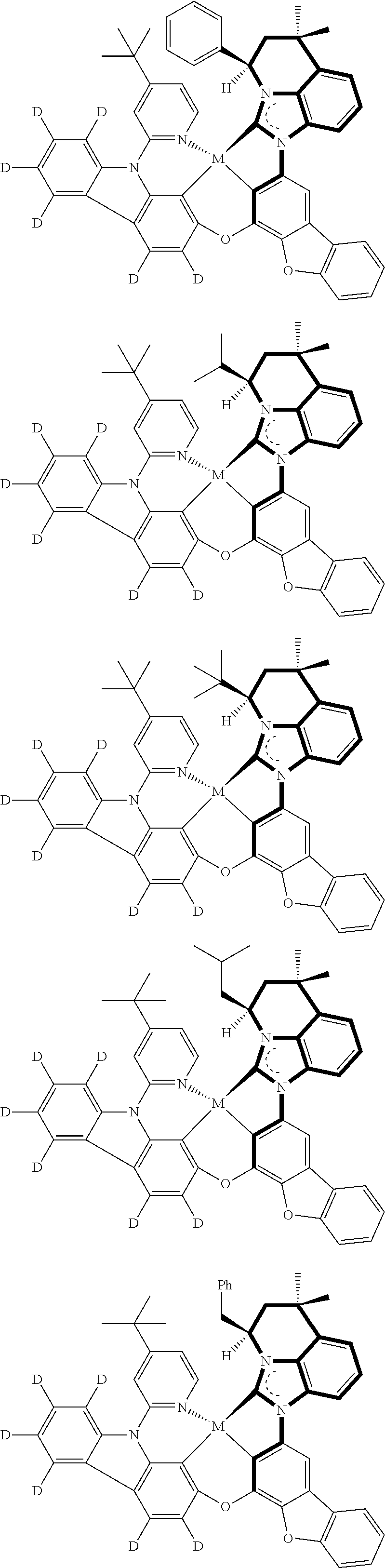

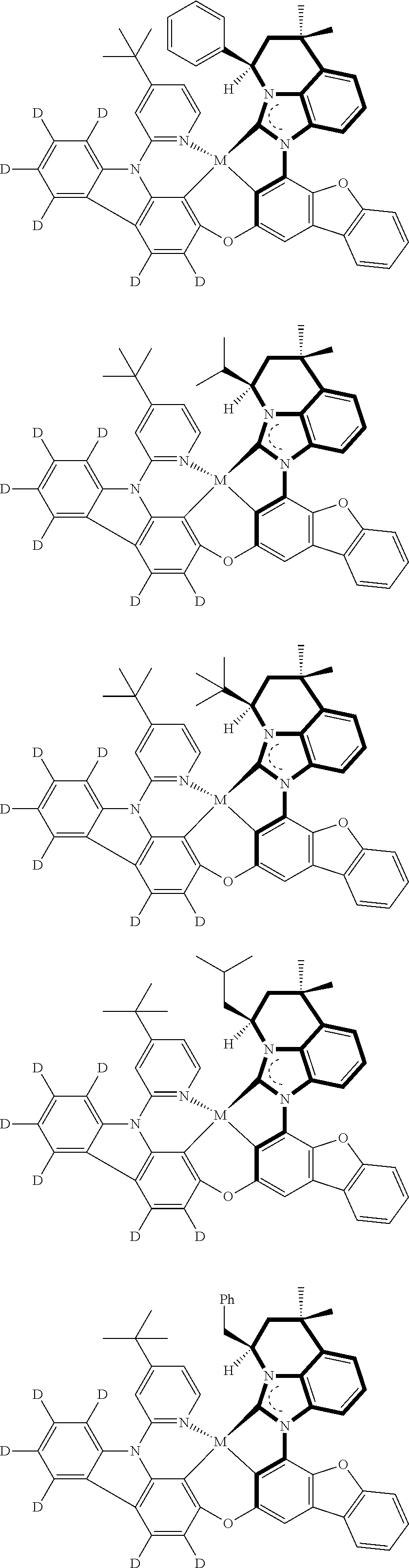

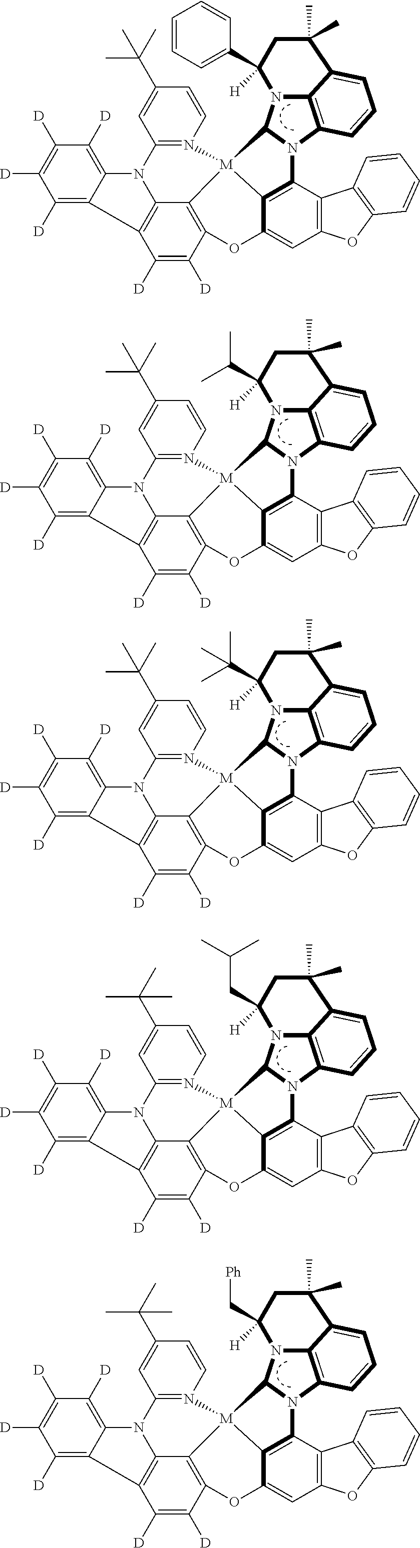

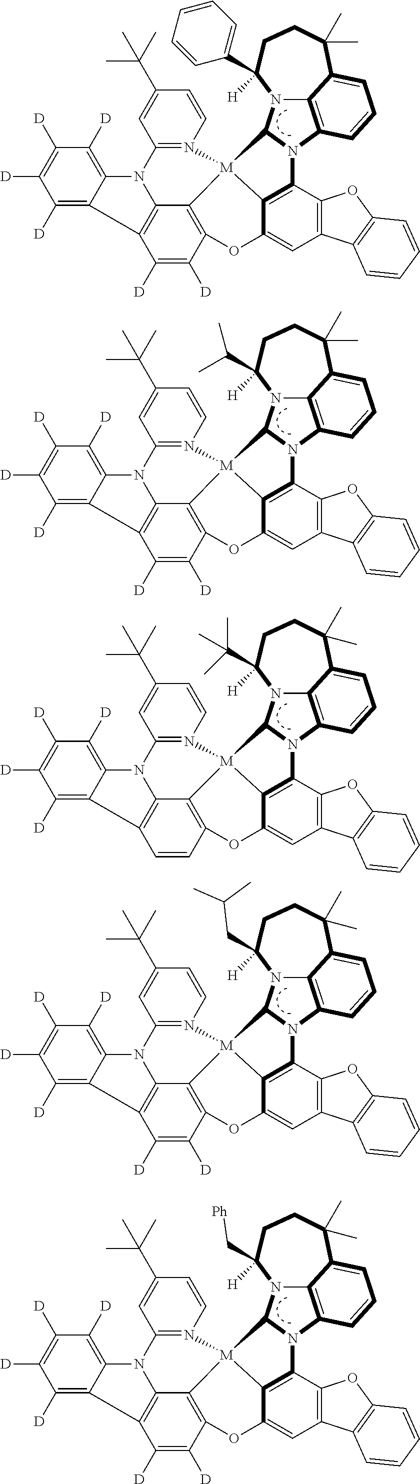

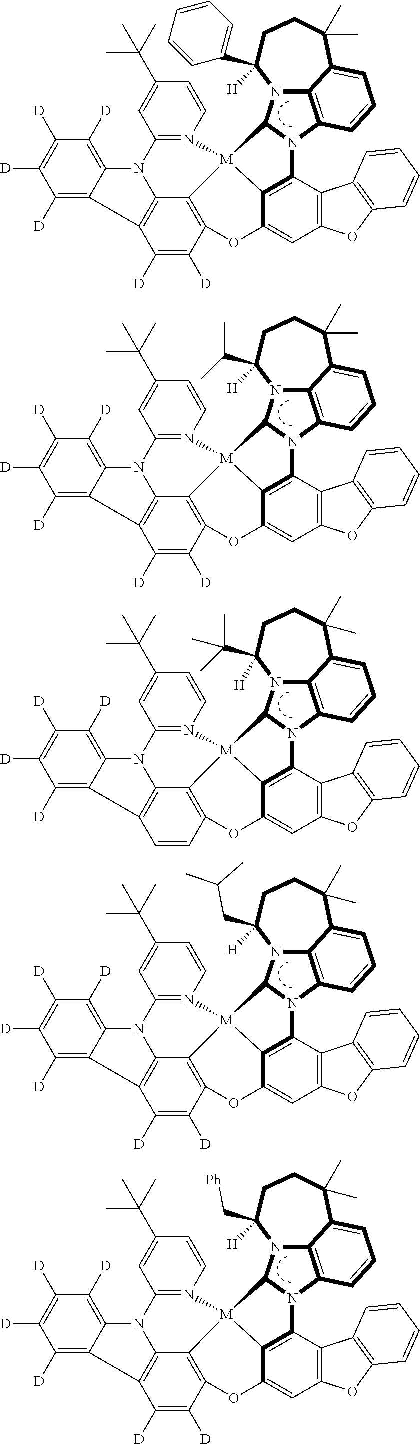

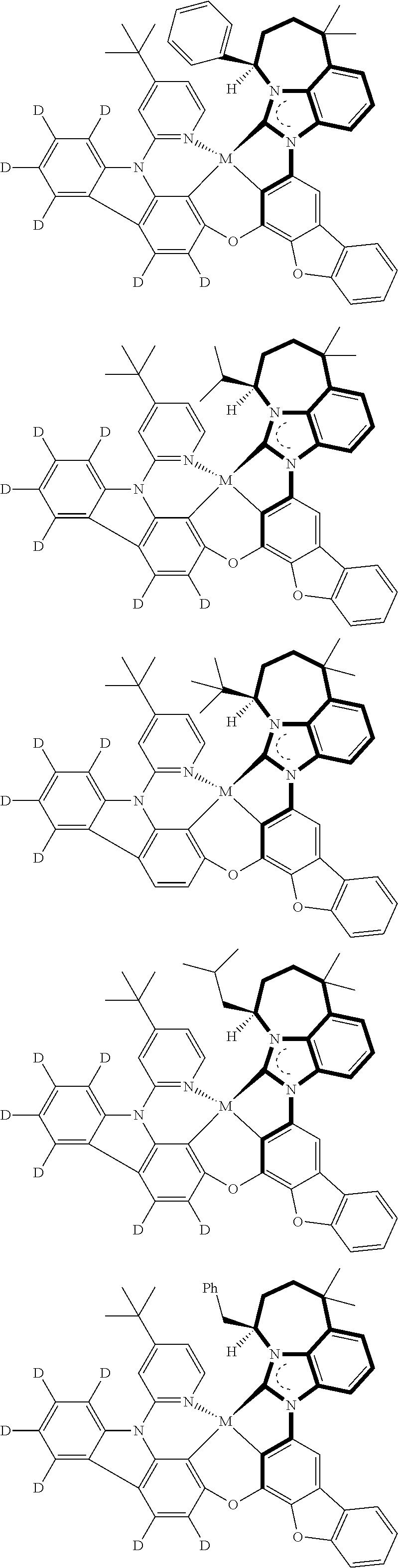

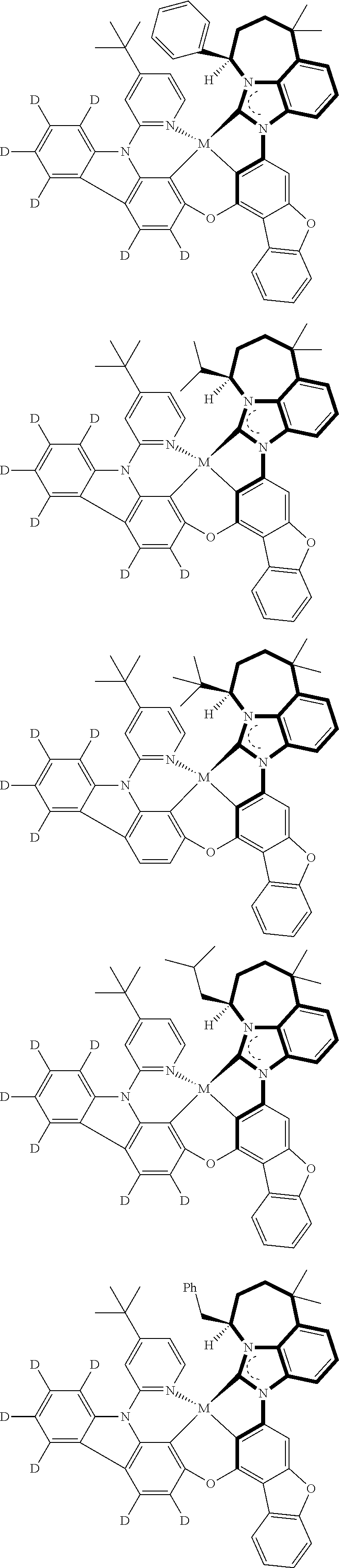

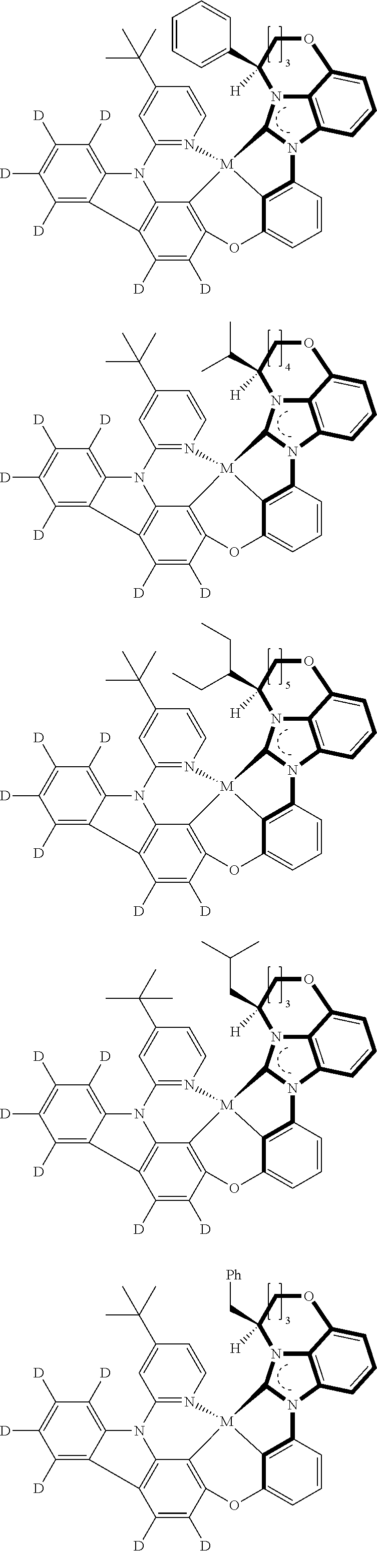

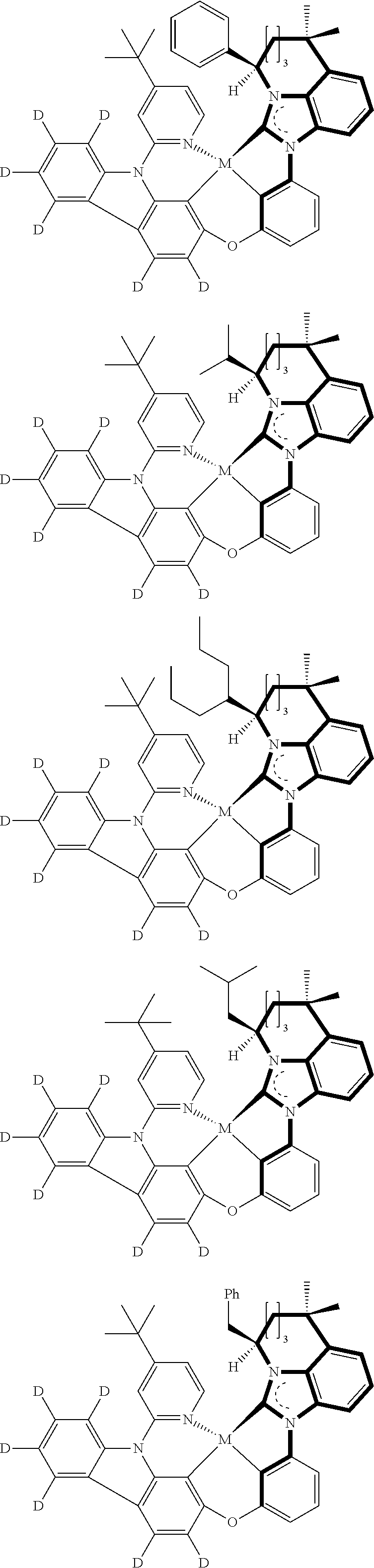

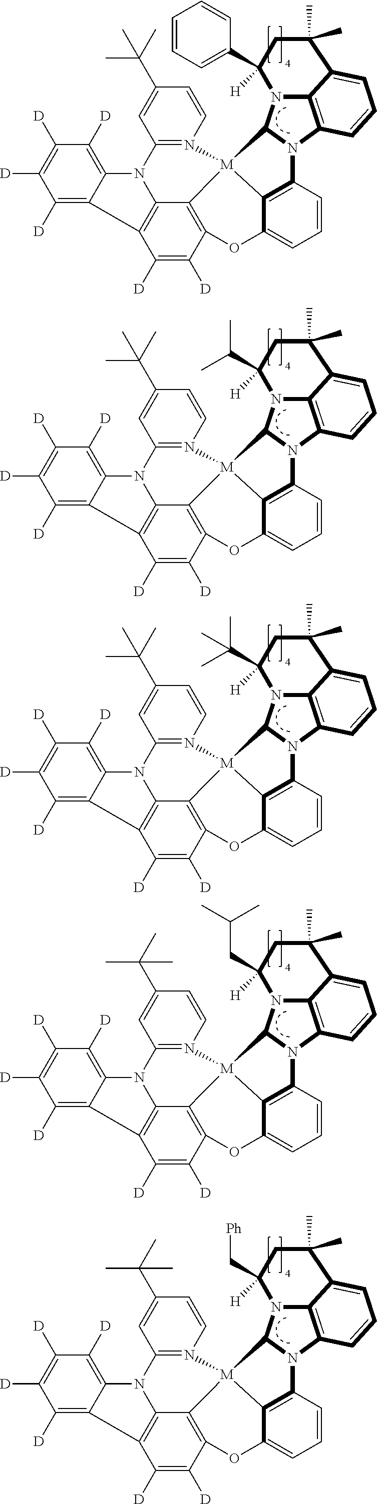

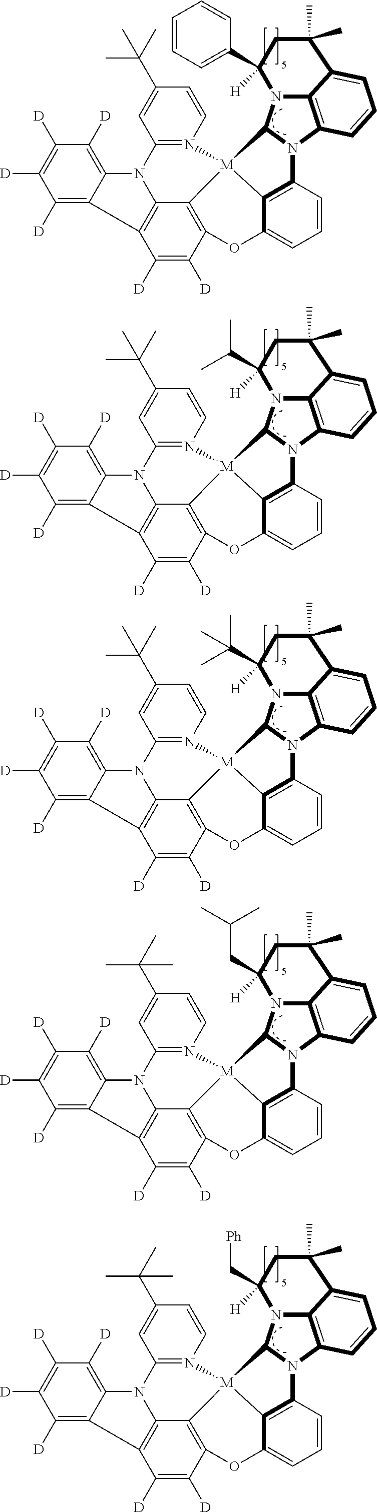

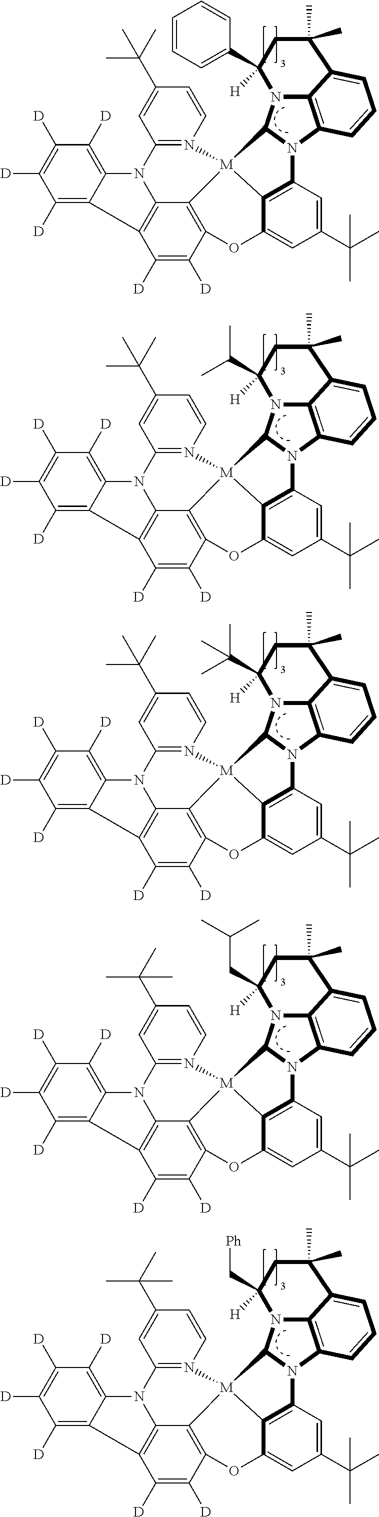

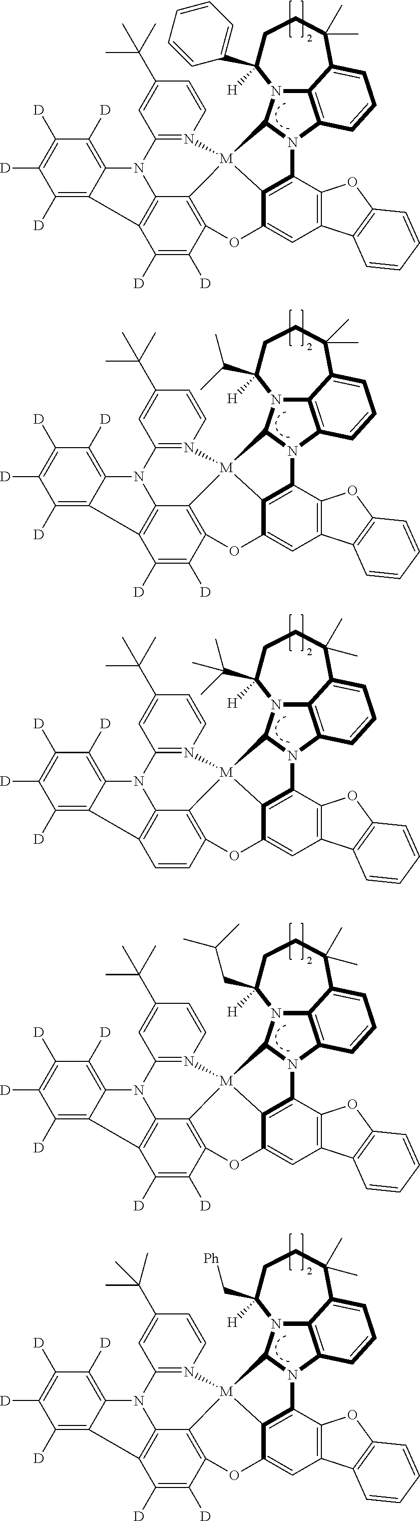

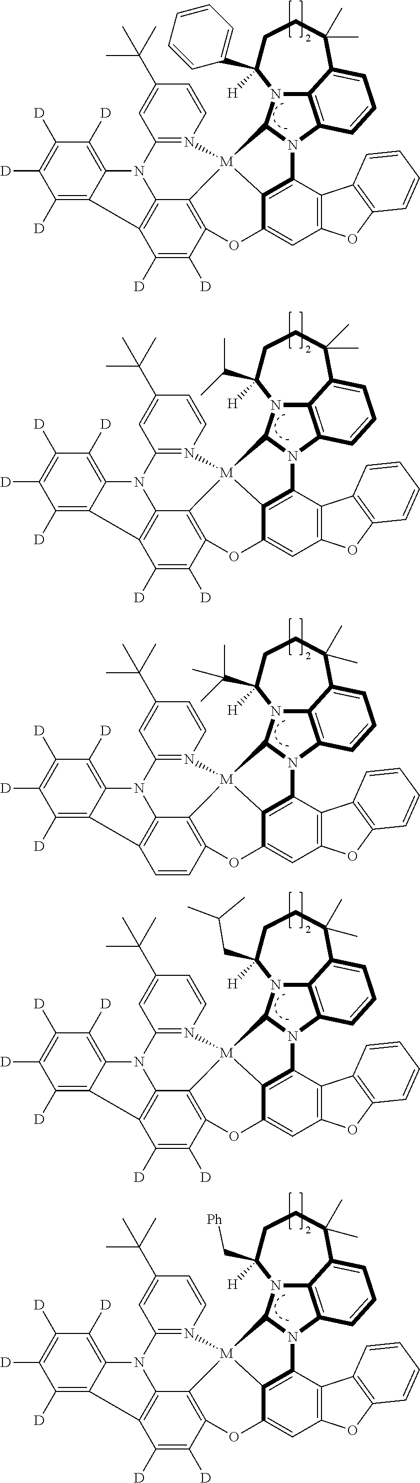

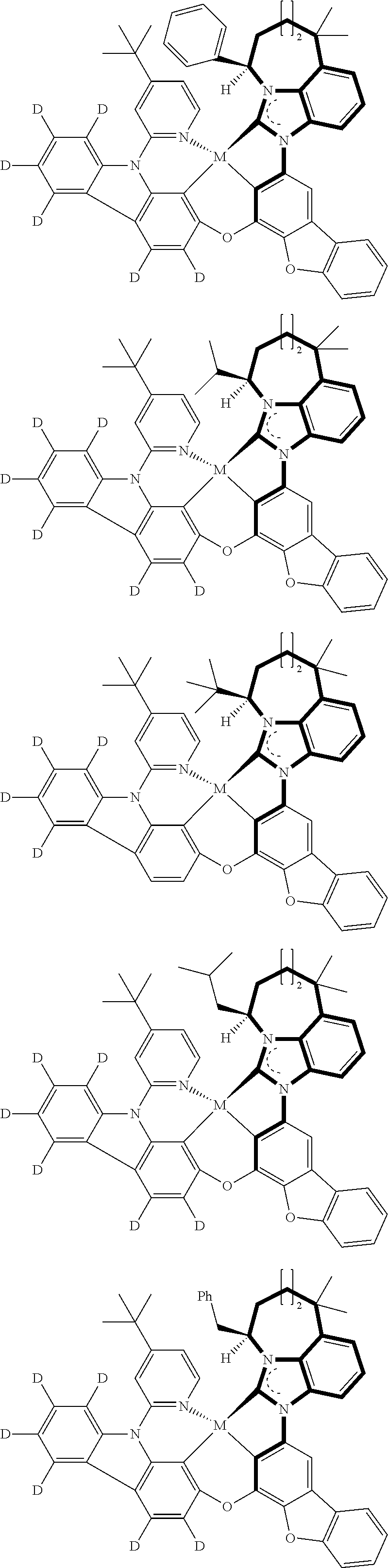

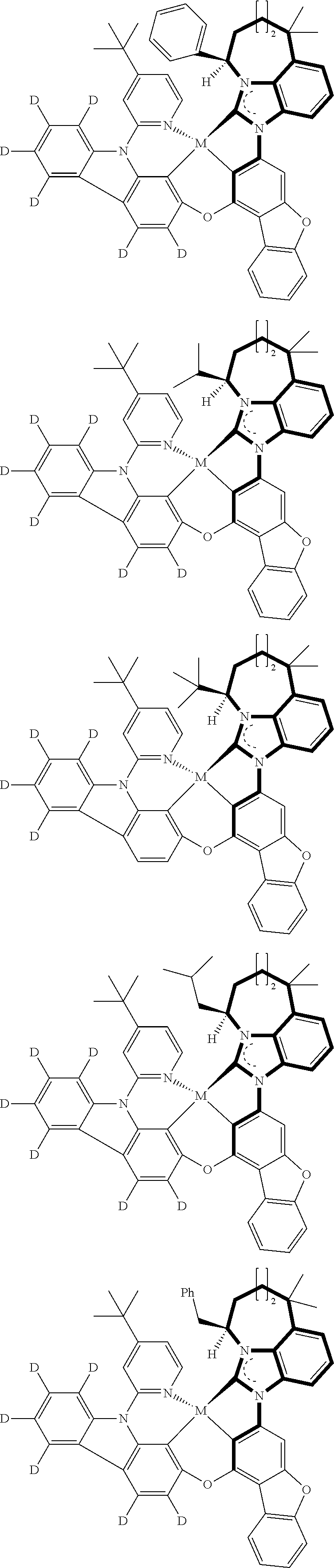

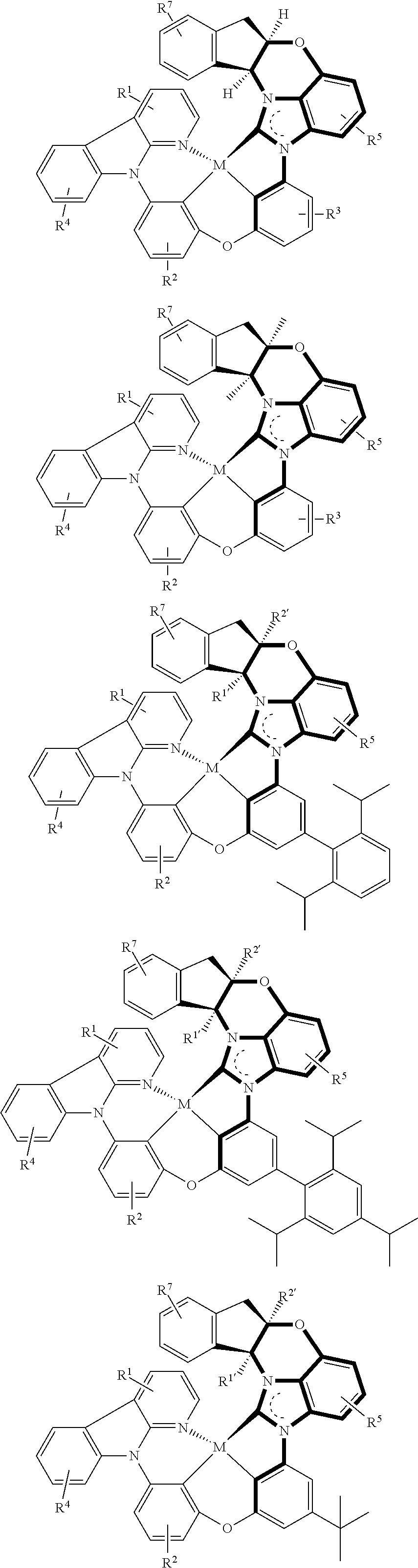

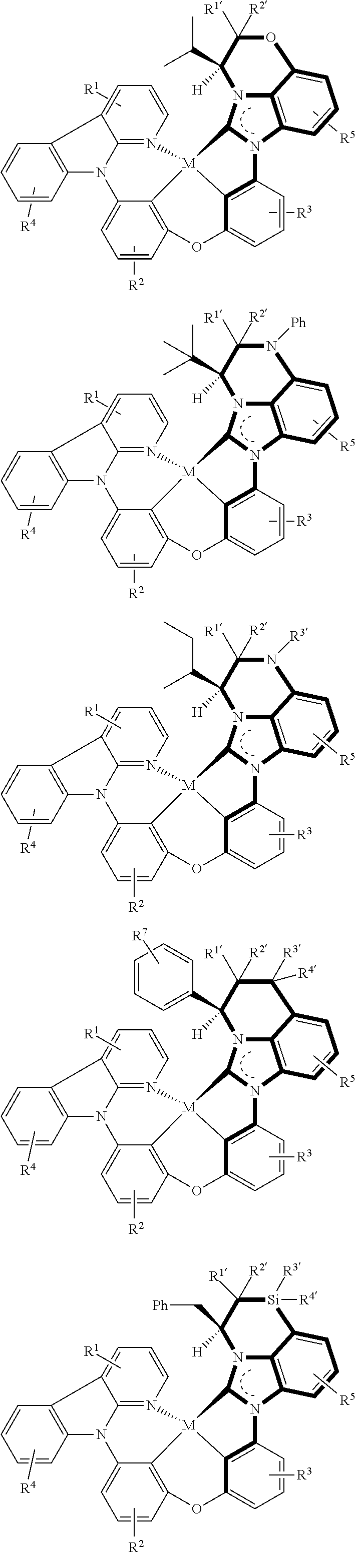

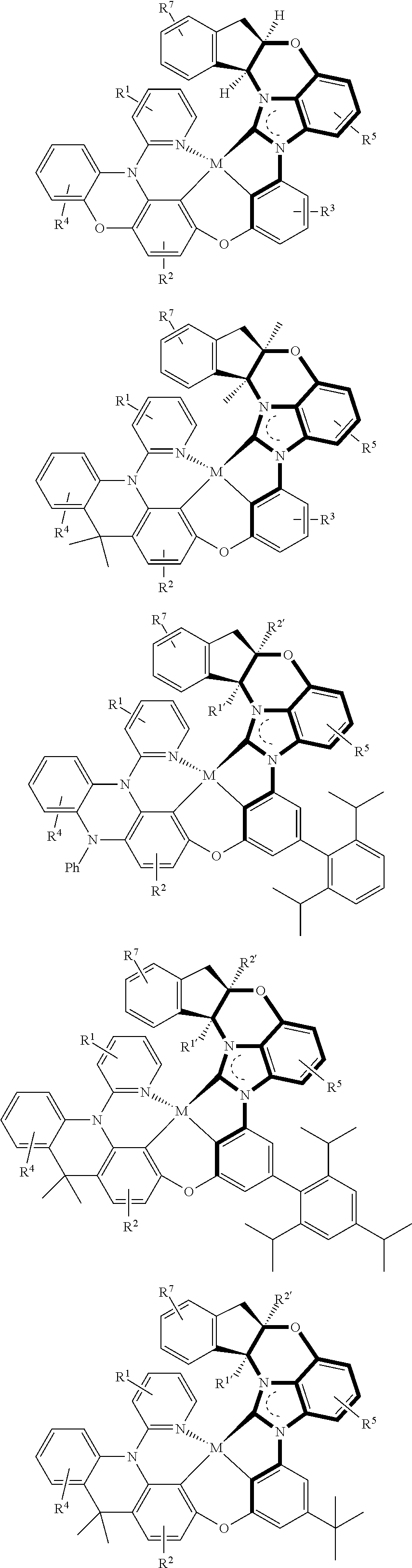

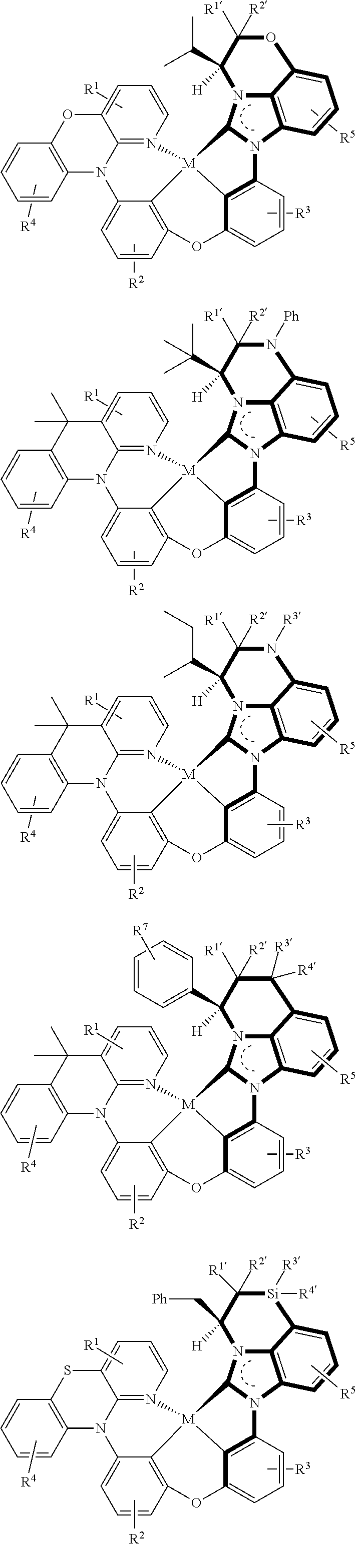

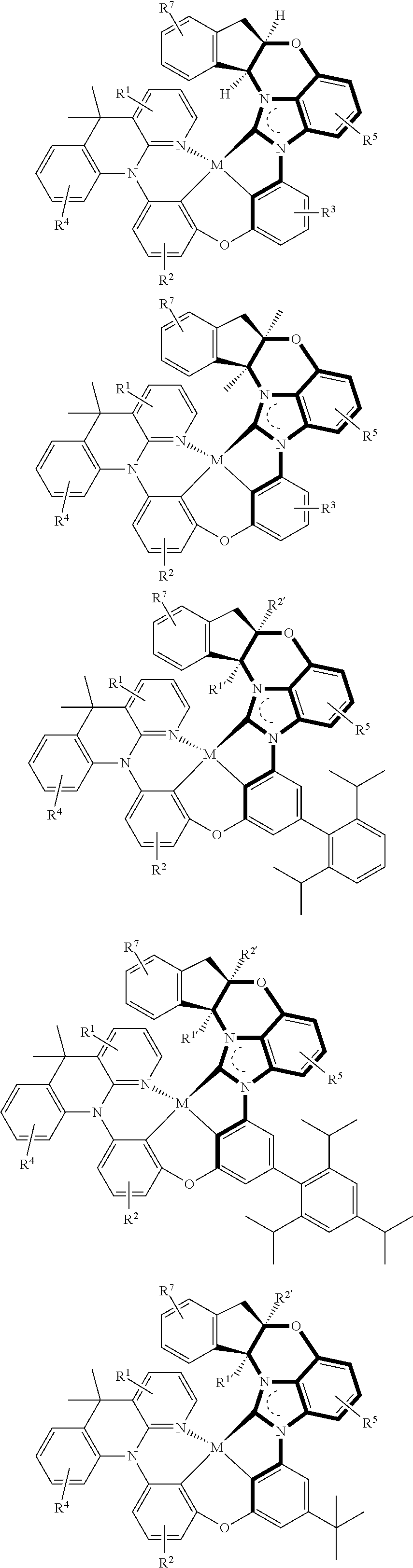

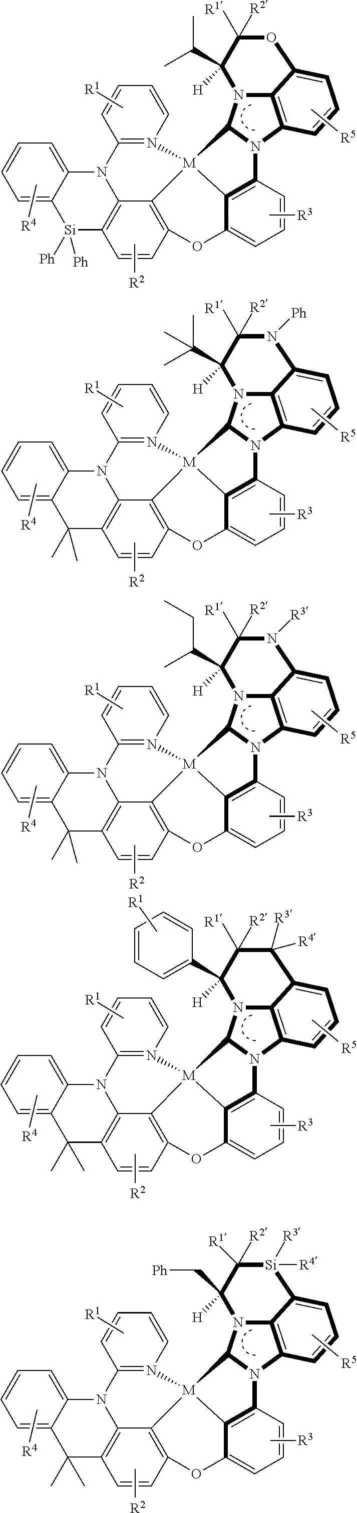

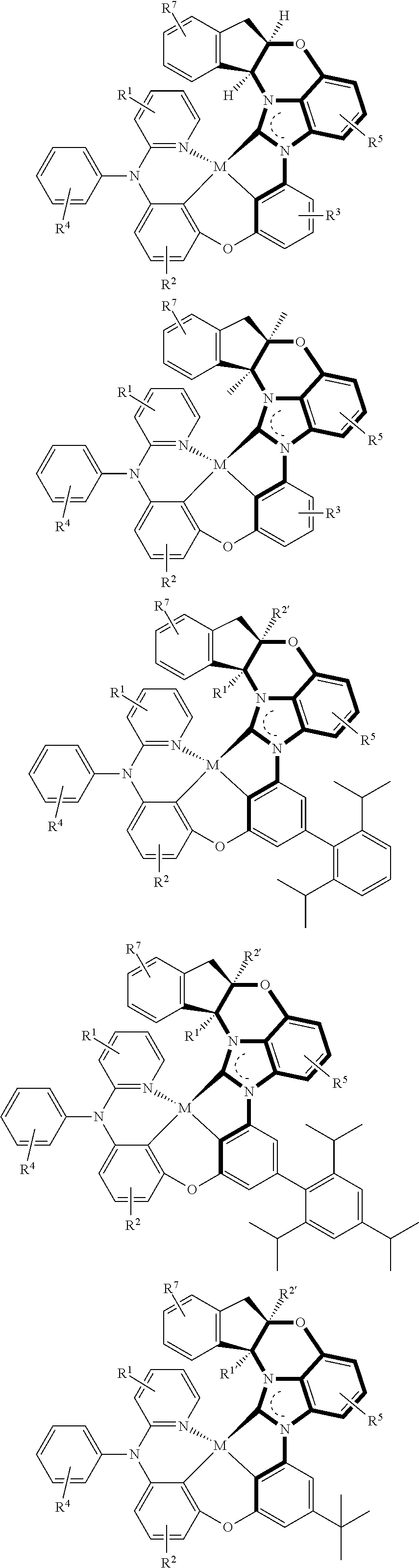

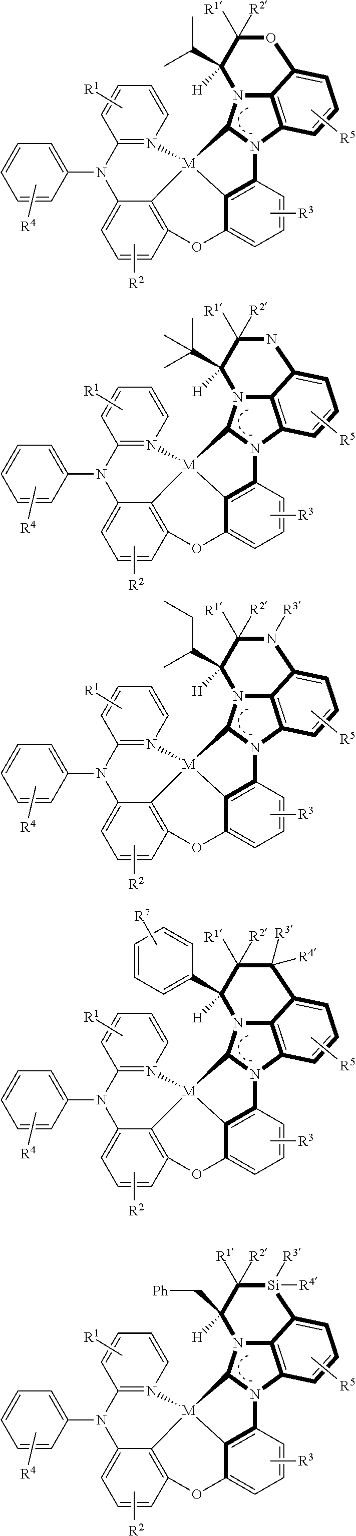

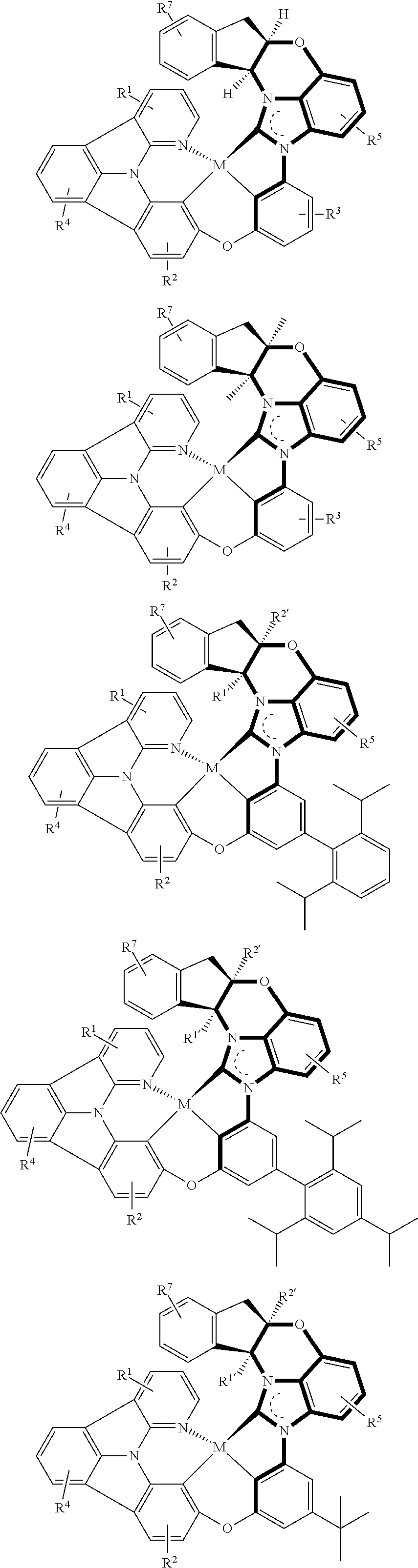

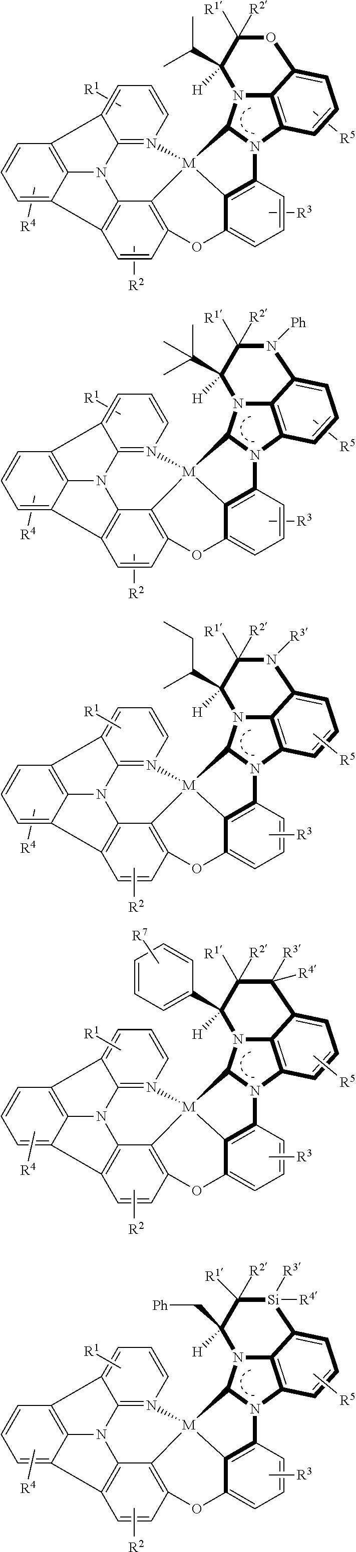

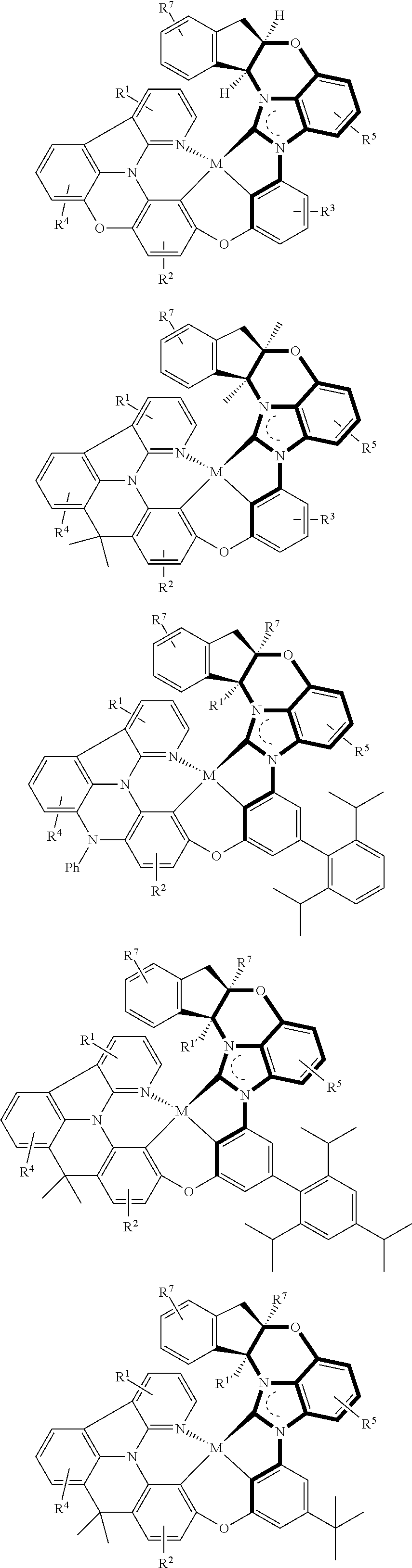

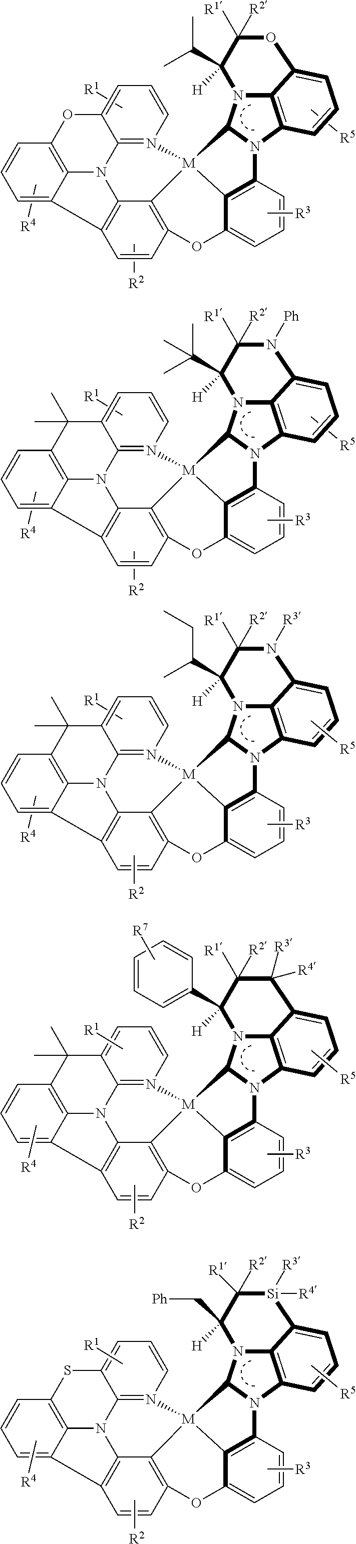

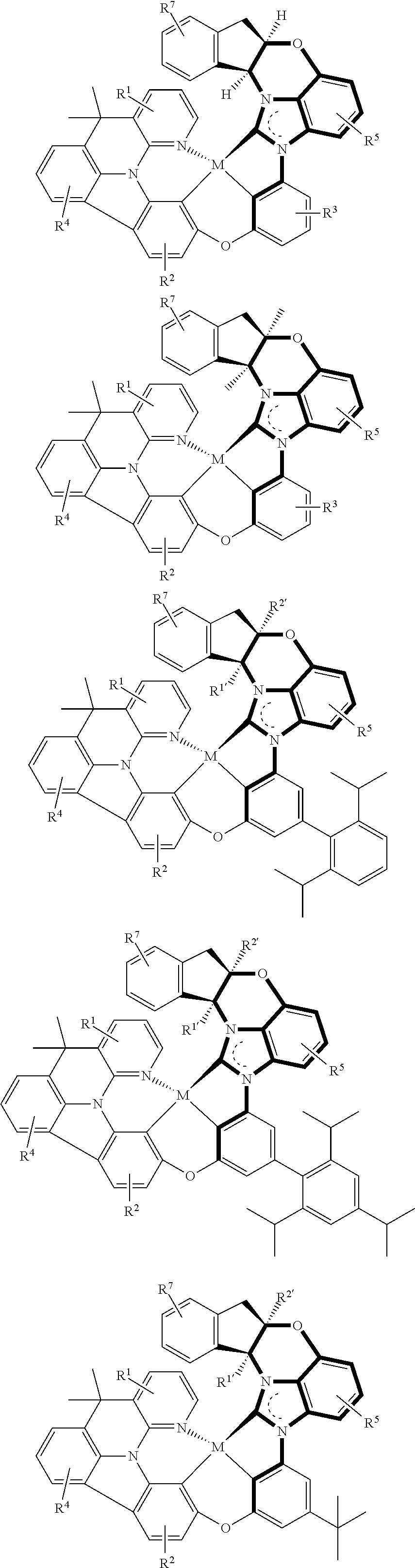

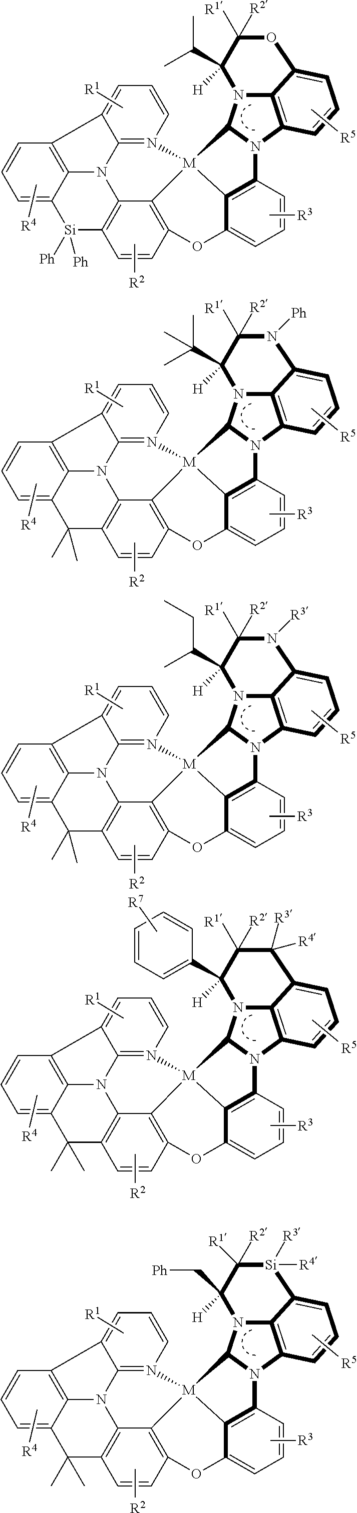

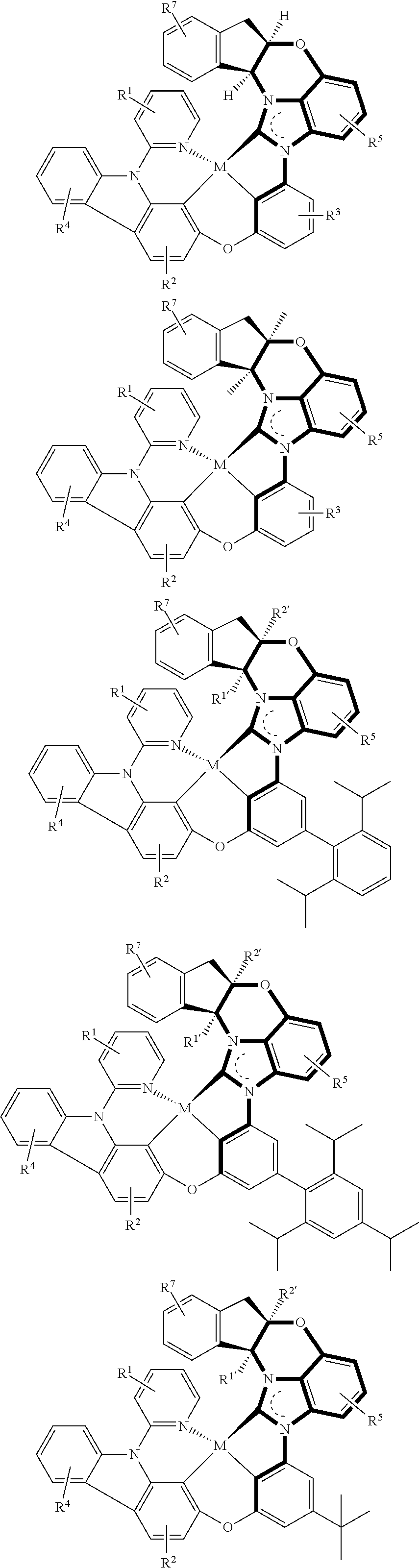

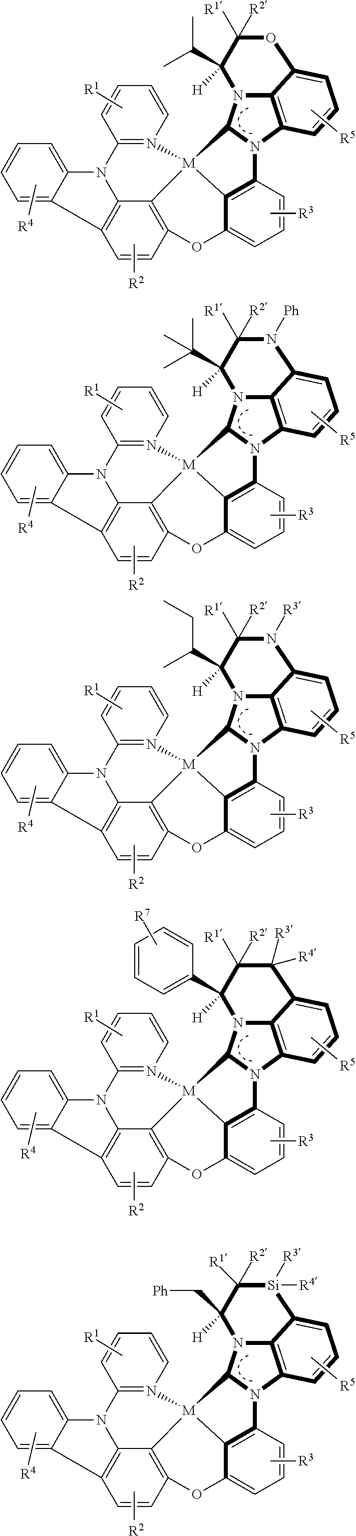

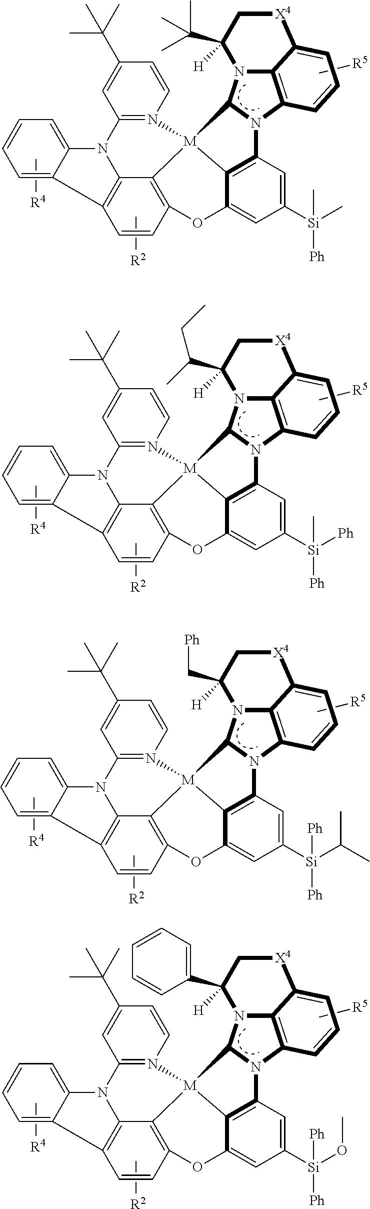

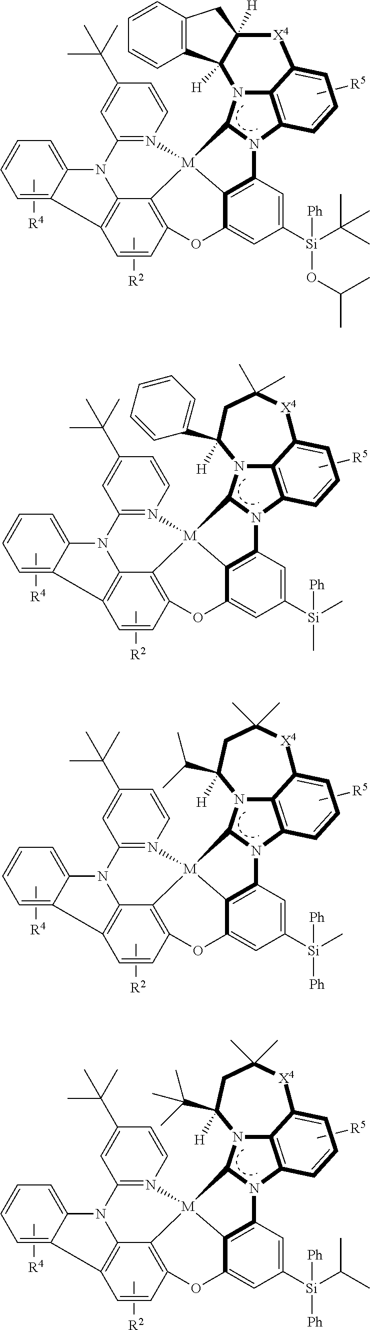

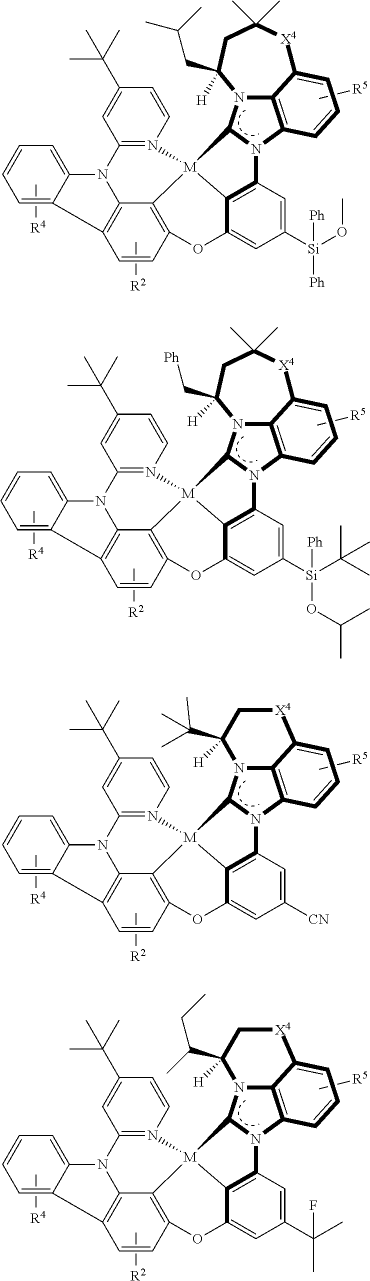

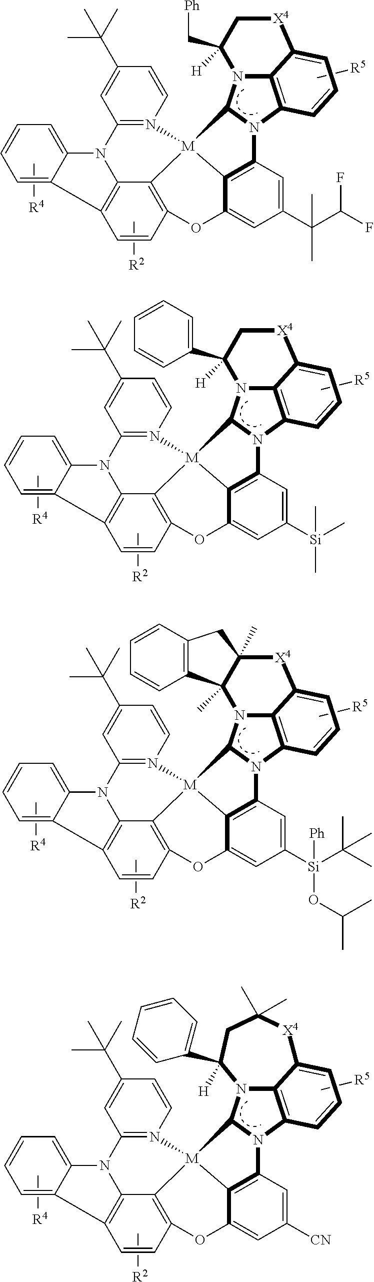

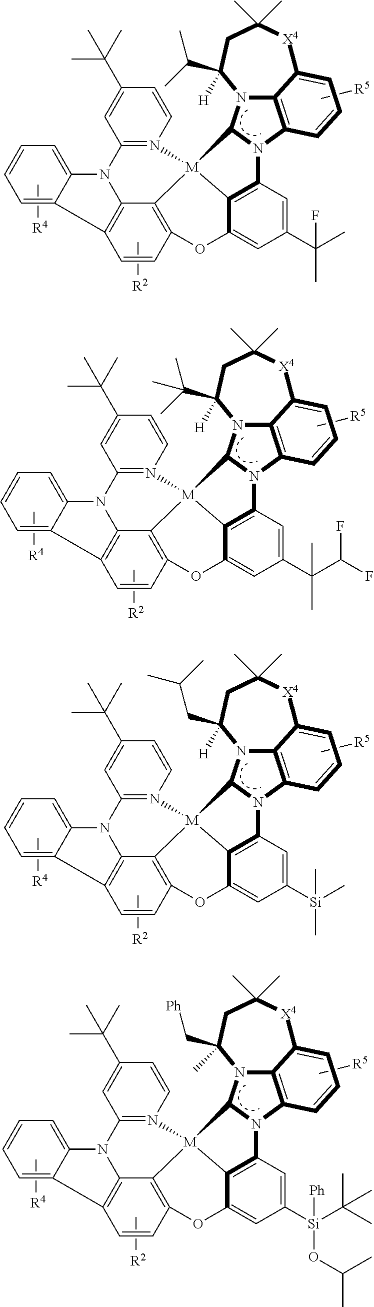

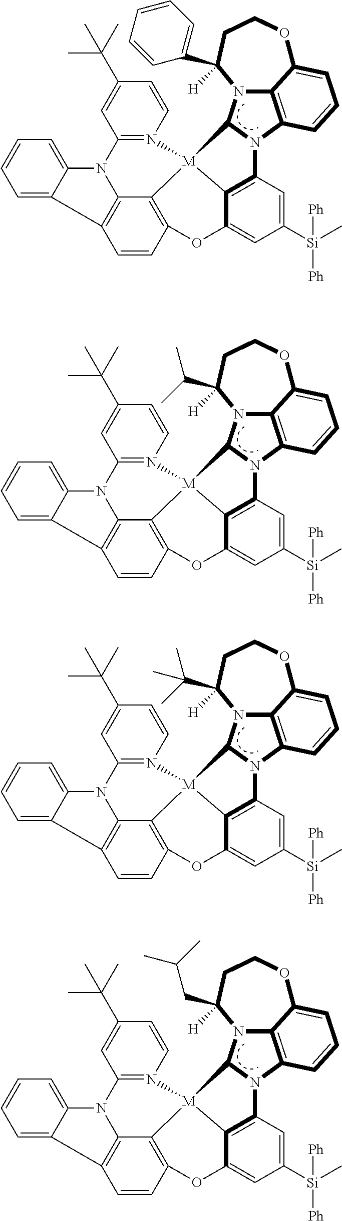

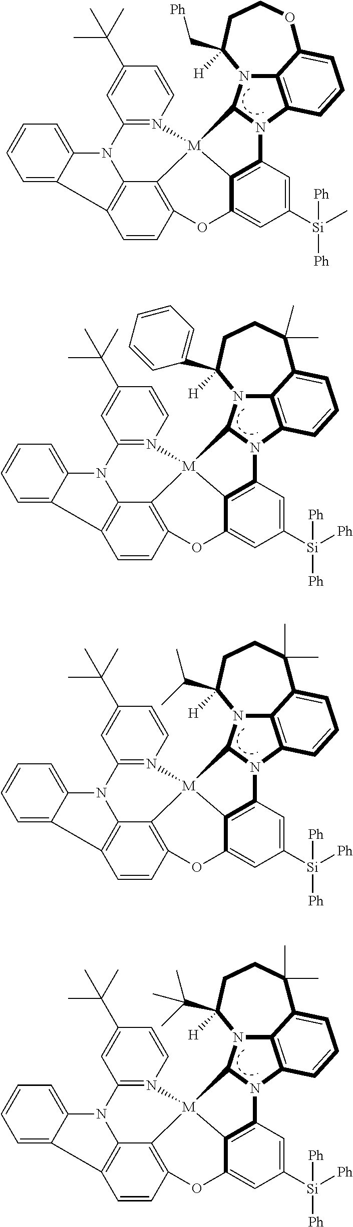

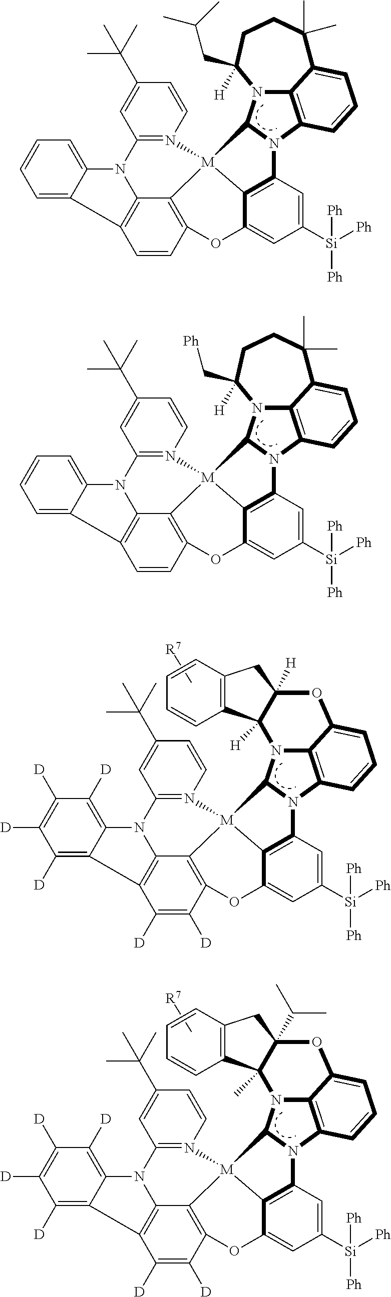

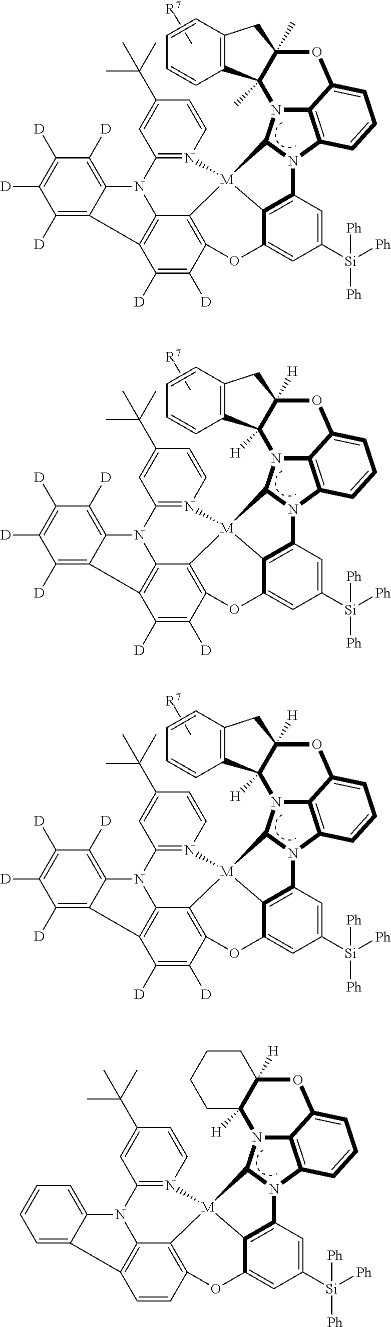

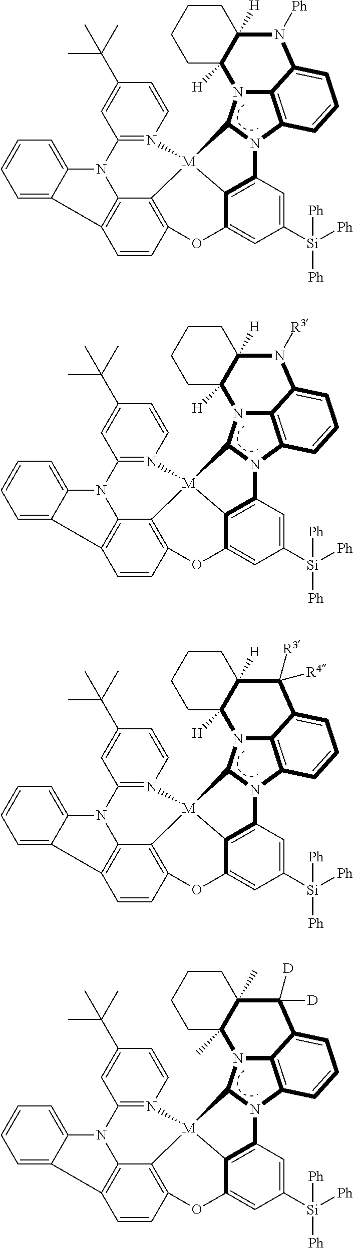

8. Use of the helically chiral platinum (II) and palladium (II) complex circularly polarized luminescent material according to claim 1 in organic light-emitting devices, 3D display devices, 3D imaging devices, optical information encryption devices, information storage devices, and biological imaging devices.

9. The use according to claim 8, wherein the organic light-emitting element is an organic light-emitting diode, a light-emitting diode, or a light-emitting electrochemical cell.

10. The use according to claim 9, wherein the luminescent element comprises a first electrode, a second electrode, and an organic layer disposed between the first electrode and the second electrode, wherein the organic layer comprises at least a helically chiral platinum(II) or palladium(II) complex circularly polarized luminescent material.

11. A display device comprising an organic light-emitting element, characterized in that the organic light-emitting element comprises a first electrode, a second electrode, and an organic layer disposed between the first electrode and the second electrode, wherein the organic layer comprises one or more helically chiral platinum(II) or palladium(II) complex circularly polarized luminescent materials as claimed in claim 1.
